(19) United States
(12) Patent Application Publication
(10) Pub. No.: US 2025/0074904 A1
(43) Pub. Date: Mar. 6, 2025

(54) NSD2-TARGETED CHECMICAL DEGRADERTS AND COMPOSITIONS AND METHODS OF USE THEREOF

(71) Applicants: THE UNIVERSITY OF NORTH CAROLINA AT CHAPEL HILL, Chapel Hill, NC (US); Pinnacle Hill, LLC, New York, NY (US)

(72) Inventors: Jon Loren Collins, Durham, NC (US); Ronan Patrick Hanley, Allston, MA (US); Lindsey Ingerman James, Chapel Hill, NC (US); John Raymond Tabor, Chapel Hill, NC (US); Andrew W. Stamford, Chatham, NJ (US)

(21) Appl. No.: 18/707,386

(22) PCT Filed: Nov. 17, 2022
(86) PCT No.: PCT/US2022/050239

§ 371 Date: May 3, 2024

Related U.S. Application Data
(60) Provisional application No. 63/280,237, filed on Nov. 17, 2021.

(51) Int. Cl. C07D 417/12; A61K 31/538; C07D 265/36; C07D 413/12

(52) U.S. Cl. CPC C07D 417/12; A61K 31/538; C07D 265/36; C07D 413/12

(57) ABSTRACT

The current invention relates to nuclear receptor-binding SET domain-containing 2 (NSD2)-targeted protein degradation reagents and pharmaceutical compositions thereof and their utility as anti-cancer agents.

### CROSS-REFERENCE TO RELATED APPLICATIONS

The present application is a U.S. National Stage entry of International Application No. PCT/US2022/050239, filed Nov. 17, 2022, which claims priority to U.S. Provisional Patent Application No. 63/280,237, filed on Nov. 17, 2021, wherein the entire contents of both applications are incorporated herein by reference.

### GOVERNMENT SUPPORT

This invention was made with government support under Grant No. CA242305 awarded by the National Institutes of Health. The government has certain rights in the invention.

### FIELD OF THE INVENTION

The present invention is directed to nuclear receptor-binding SET domain-containing 2 (NSD2)-targeted protein degradation ligands and pharmaceutical compositions thereof and their utility as anti-cancer agents.

### BACKGROUND

Nuclear receptor-binding SET domain-containing 2 (NSD2, also known as multiple myeloma SET domain (MMSET) and Wolf-Hirschhorn syndrome candidate 1 (WHSC1)) is a protein lysine methyltransferase that belongs to the NSD family, which also includes NSD1 and NSD3. The main function of NSD2 is the production of the bulk of H3K36me2 in diverse cell types. Dimethylation of H3K36 by both NSD1 and NSD2 recruits DNMT3 a at intergenic regions to control DNA methylation and regulate development and homeostasis (Weinberg, D. N., et al., Nature, 573, 281-286, 2019). NSD2 is also required for efficient non-homologous end-joining and homologous recombination, two canonical DNA repair pathways (Shah, M. Y. et al. Oncogene, 35, 5905-5915, 2016; Zhang, J. et al., Cancer Discov., 9, 1306-1323).

Structurally, NSD2 has multiple protein-protein interaction (PPI) modules with known or potential chromatin reading functions, including five PHD (plant homeodomain) and two PWWP (proline-tryptophan-tryptophan-proline) domains (Bennet, R. L. et al. Cold Spring Harb Perspect Med., 7, 2017), as well as a putative DNA-binding HMG-box (high mobility group box) domain in addition to its catalytic domain (FIG. 1). Mounting evidence suggests that these domains play important roles in NSD2 function, but the individual and/or collective roles of the NSD2 chromatin reader domains are still being elucidated. Many PWWP domains are known H3K36me2,3 reading modules that engage methyl-lysine while simultaneously interacting with nucleosomal DNA adjacent to H3K36 (Qin, S. et al. Trends Biochem. Sci., 39, 536-547, 2014; Vermeulen, M. et al. Cell 142, 967-980, 2010). The isolated N-terminal PWWP domain of NSD2 (NSD2-PWWP1) binds H3K36 di- and trimethylated nucleosomes; this interaction presumably is mediated by a conserved aromatic cage and stabilizes NSD2 at chromatin (Sankaran, S. M. et al., J. Biol. Chem. 291, 8465-8474, 2016). Mutation of the aromatic cage residues abrogates NSD2-PWWP1 binding to nucleosomal H3K36me2, but has only modest effect on global H3K36 methylation level in cells. However, H3K36 methylation has been shown to be abolished upon mutation of the second PHD domain (PHD2)(Huang, Z. et al. Cancer. Res. 73, 6277-6288, 2013).

Recently, a growing number of studies have linked NSD methyltransferases to a variety of diseases and cancers. For example, NSD2 is aberrantly expressed, amplified or somatically mutated in multiple types of cancer, leading to increased methylation of lysine 36 of histone 3 (H3K36) (Kuo, A. J., et al. Mol. Cell, 44, 609-620, 2011) and subsequent proliferation. In particular, the t(4;14)NSD2 translocation in multiple myeloma (MM) and the hyper-activating NSD2 E1099K mutation in a subset of pediatric acute lymphoblastic leukemia (ALL) both result in altered chromatin methylation which drives oncogenesis (Keats, J. J. et al., Blood, 101, 1520-1529, 2003; Oyer, J. A. et al., Oncogene, 38, 6710686, 2019).

As such, high expression of the NSD2 protein has been demonstrated in different human cancer types, including bladder, brain, gastrointestinal, lung, liver, ovary, skin, uterus, breast, prostrate and glioblastoma. (Coussens et al., J. Biol. Chem. 293, 13750-13654 (2018); Ezponda et al., Oncogene 32: 2882-2890 (2013)). Notably, NSD2 is among the most frequently mutated genes in pediatric cancer genomes. The NSD2 SET domain variant, E1099K, was identified in both acute lymphoblastic leukemia tumors and cell lines with increased H3K36me2 that lack the t (4; 14) translocation. NSD2 is also among the most frequently mutated genes found in mantle cell lymphoma tumors, where both E1099K and T1150A variants are observed. The E1099K variant has also been reported in chronic lymphocytic leukemia (CLL), lung and stomach cancers. In general, NSD2 upregulation is associated with aggressive tumor behavior and poor prognosis.

Due to growing occurrence of drug resistance to current traditional anticancer drugs employed in treating such cancer, there has been an ongoing need for improving such anti-cancer therapies and/or developing new treatment options, which can act via different cellular mechanisms to combat cancers. One of such new treatment options involves the NSD2 enzyme, which represents a promising target for anti-cancer therapy.

### SUMMARY

Provided herein are compounds, which can bind to the NSD2 enzyme in the non-active site and degrade the enzyme. Thus, one aspect of the current disclosure is directed to a compound of Formula (I):

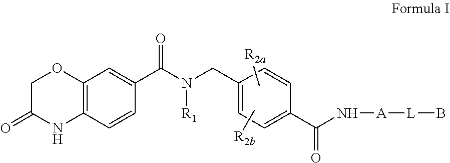

or an enantiomer, an enantiomeric mixture, or a pharmaceutically acceptable salt thereof, wherein:
   R₁ is -cyclopropyl or -isopropyl;
   R₂ₐ and R_{2b} are independently selected from hydrogen, C1-C6 alkyl, C1-C6 haloalkyl, C1-C6 alkoxy, halogen, —CN, —NH₂, and —OH;
   A is absent or present, and when present is selected from

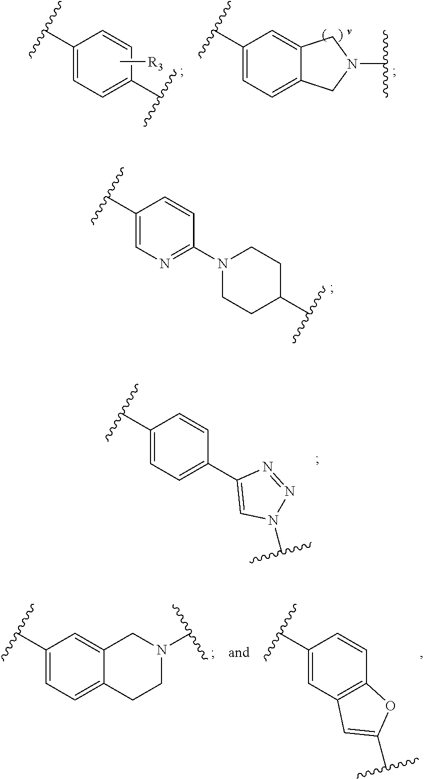

wherein R₃ is selected from hydrogen, C1-C6 alkyl, C1-C6 haloalkyl, C1-C6 alkoxy, halogen, —CN, —NH₂, and —OH, and v is 1 or 2;
L is

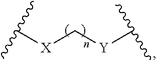

wherein X is selected from —C(═O)—, —CH₂—, C(═O)O—, —C≡C—, —O—, —S—, —NH—, —S(═O)—, —C(═O)NH—, —C(═O)NCH₃—, and

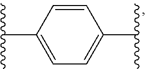

wherein Y is absent or is selected from —O—, —CH₂—, —NH—, —NCH₃—, —[OCH₂CH₂]ₘ—,

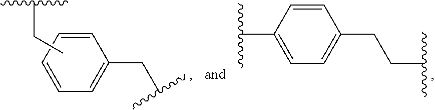

wherein m is an integer selected from 1, 2, 3, 4, and 5, and wherein n is an integer selected from 0, 1, 2, 3, 4, 5, 6, 7, 8, 9, and 10; and
B is selected from hydrogen, —NH₂, —NH(CH₃)—C(═O)(C1-C6 alkyl), —C(═O)(C1-C6 haloalkyl), C1-C6 alkyl, —O(C1-C6 alkyl), —COOH, —OH, —NH(C1-C6 alkyl), —N(C1-C6 alkyl)₂,

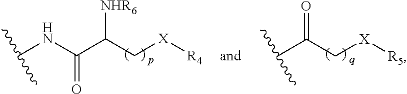

wherein p and q are an integer independently selected from 0, 1, 2, 3, 4, 5, 6, 7, 8, 9, and 10, X is selected from —CH₂—, —NH—, and —O—, R₆ is hydrogen or —C(═O)CH₃—, and R₄ and R₅ are independently selected from hydrogen, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

Another aspect of the disclosure is directed to a pharmaceutical composition comprising a compound as disclosed herein or a pharmaceutically acceptable salt thereof, and one or more pharmaceutically acceptable carrier(s). In some embodiments, the disclosed compound can be a prodrug.

Another aspect of the disclosure is directed to a method for treating a disease or condition that is treatable by inhibition of nuclear SET-domain-containing protein (NDS2), the method comprising administering to a subject in need thereof a therapeutically effective amount of a compound, prodrug, or a pharmaceutical composition as disclosed herein. In some embodiments, the disease is cancer.

### DESCRIPTION OF THE DRAWINGS

FIG. 1 shows the NSD2 protein architecture of the three splicing isoforms NSD2-long, NSD2-short, and REIIBP.

FIG. 2 shows an exemplary general chemical structure of a NSD2 degrader disclosed herein containing a NSD2 binding moiety, a linker (L) and an (un)natural amino acid moiety (B).

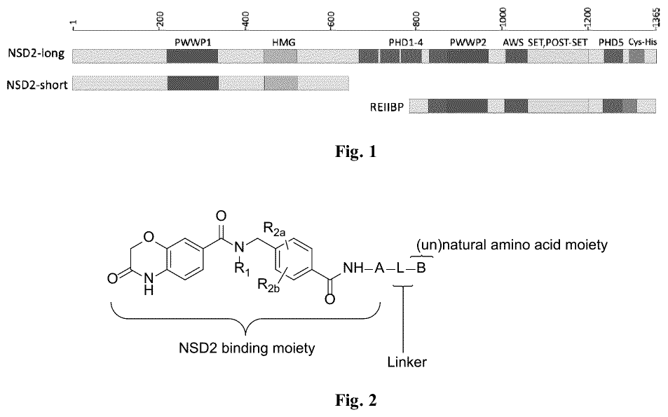

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

NSD2 (nuclear receptor-binding SET domain-containing 2) is a key player in epigenetic regulation, known for its ability to mono- and dimethylate lysine 36 of histone 3 (H3K36). This mark is associated with active transcription, and elevated levels of H3K36me2 lead to adherent activation of normally silenced genes. Consequently, NSD2 is a potent oncoprotein and has been implicated as a therapeutic target for a variety of cancers. Recent research efforts suggest that in addition to its catalytic SET domain, NSD2 contains several PWWP and PHD methyl-lysine (Kme) reader domains, which are thought to be critical in propagating H3K36me2 and recruiting NSD2 to its oncogenic genes. Various NSD2 antagonists have been prepared to date (WO2021/026803; WO2021/028854; and Dilworth, D. et al., bioRxiv, Mar. 7, 2021).

However, since drug resistance is the main limitation for cancer therapy, other treatment strategies are needed, such as compounds that block NSD2 enzymatic activity or NSD2 degraders. Thus, in some embodiments, the NSD2 degraders disclosed herein comprise an NSD2 binding moiety and an (un)natural amino acid moiety connected by a linker (see FIG. 2). In some embodiments, the (un)natural amino acid moiety is absent and the NSD2 degraders comprise an NSD2 binding moiety and the linker. Such NSD2 targeted compounds exhibit potent NSD2 binding affinity while simultaneously demonstrating efficient degradation of the target protein (i.e., NSD2). Pharmaceutical compositions containing these NSD2 degraders and methods of use thereof are described further in more detail below.

### I. Definitions

As used herein, the term “alkyl group” refers to a saturated hydrocarbon radical containing 1 to 8, 1 to 6, 1 to 4, or 5 to 8 carbons. In some embodiments, the saturated radical contains more than 8 carbons. An alkyl group is structurally similar to a noncyclic alkane compound modified by the removal of one hydrogen from the noncyclic alkane and the substitution therefore of a non-hydrogen group or radical. Alkyl group radicals can be branched or unbranched. Lower alkyl group radicals have 1 to 4 carbon atoms. Higher alkyl group radicals have 5 to 8 carbon atoms. Examples of alkyl, lower alkyl, and higher alkyl group radicals include, but are not limited to, methyl, ethyl, n-propyl, isopropyl, n-butyl, sec butyl, t butyl, amyl, t amyl, n-pentyl, n-hexyl, i-octyl and like radicals.

As used herein, the designations “C(═O),” “CO” and “C(O)” are used to indicate a carbonyl moiety. Examples of suitable carbonyl moieties include, but are not limited to, those found in ketones and aldehydes.

The term “cycloalkyl” refers to a hydrocarbon with 3-8 members or 3-7 members or 3-6 members or 3-5 members or 3-4 members and can be monocyclic or bicyclic. The ring may be saturated or may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-10 membered ring systems where the heteroatoms are selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, isoquinolinyl, indazolyl, and the like.

As used herein, the term “alkoxy” refers to a moiety of the formula —ORa where Ra is an alkyl group as defined herein containing one to twelve carbon atoms. Unless stated otherwise specifically in the specification, an alkoxy group is optionally substituted.’

As used therein, the term “haloalkyl” refers to an alkyl group, as defined herein, that is substituted by one or more halo atoms, as defined herein, e.g., trifluoromethyl, difluoromethyl, fluoromethyl, trichloromethyl, —CH₂CF₃, —CH₂CHF₂, —CH₂CH₂F, —CHFCF₃, —CHFCHF₂, —CHFCH₂F, —CHFCH₃, —CF₂CF₃, —CF₂CHF₂, —CF₂CH₂F, —CF₂CH₃, —CH₂CF₂CH₃, —CH₂CHFCH₃, 3-bromo-2-fluoropropyl, 1,2-dibromoethyl, and the like. Unless stated otherwise specifically in the specification, a haloalkyl group is optionally substituted.

As used herein, the term “substituted” refers to a moiety (such as heteroaryl, aryl, cycloalkyl, alkyl, and/or alkenyl) wherein the moiety is bonded to one or more additional organic or inorganic substituent radicals. In some embodiments, the substituted moiety comprises 1, 2, 3, 4, or 5 additional substituent groups or radicals. Suitable organic and inorganic substituent radicals include, but are not limited to, halogen, hydroxyl, cycloalkyl, aryl, substituted aryl, heteroaryl, heterocyclic ring, substituted heterocyclic ring, amino, mono-substituted amino, di-substituted amino, acyloxy, nitro, cyano, carboxy, carboalkoxy, alkyl carboxamide, substitutedalkyl carboxamide, dialkyl carboxamide, substituted dialkyl carboxamide, alkylsulfonyl, alkylsulfinyl, thioalkyl, alkoxy, substituted alkoxy or haloalkoxy radicals, wherein the terms are defined herein. Unless otherwise indicated herein, the organic substituents can comprise from 1 to 4 or from 5 to 8 carbon atoms. When a substituted moiety is bonded thereon with more than one substituent radical, then the substituent radicals may be the same or different.

As used herein, the term “unsubstituted” refers to a moiety (such as heteroaryl, aryl, alkenyl, and/or alkyl) that is not bonded to one or more additional organic or inorganic substituent radical as described above, meaning that such a moiety is only substituted with hydrogens. It will be understood that the structures provided herein and any recitation of “substitution” or “substituted with” includes the implicit proviso that such structures and substitution are in accordance with permitted valence of the substituted atom and the substituent, and that the substitution results in a stable compound, e.g, which does not spontaneously undergo transformation such as by rearrangement, cyclization, elimination, etc.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., NSD2 inhibitor and one or more additional therapeutics) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g, toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention, which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of a treatment if such subject would benefit biologically, medically or in quality of life from such treatment.

As used herein, the term a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

As used herein, the term “anticancer agent” or antineoplastic agent, refers to a therapeutic agent that is useful for treating or controlling the growth of cancerous cells.

### II. Compounds

Provided herein are NSD2 degraders and methods of use thereof for the treatment of disease, such as cancers and other diseases dependent on the activity of NSD2.

In some embodiments, the compounds disclosed herein comprise a compound of Formula (I):

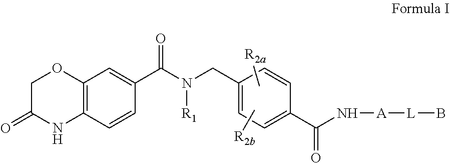

or an enantiomer, an enantiomeric mixture, or a pharmaceutically acceptable salt thereof; wherein:
R₁ is hydrogen, -cycloalkyl, -halocycloalkyl, heterocycloalkyl, -cyclopropyl, C1-C6 alkyl, C1-C6 haloalkyl, or -isopropyl;
R₂ₐ and R_{2b} are independently selected from hydrogen, deuterium, C1-C6 alkyl, C1-C6 haloalkyl, C1-C6 alkoxy, halogen, —COOH, —CO(C1-C6alkyl), —COO(C1-C6 alkyl), —OCF₃, —NO₂, —CONH₂, —CN, —NH₂, and —OH;
A is absent or present, and when present is selected from

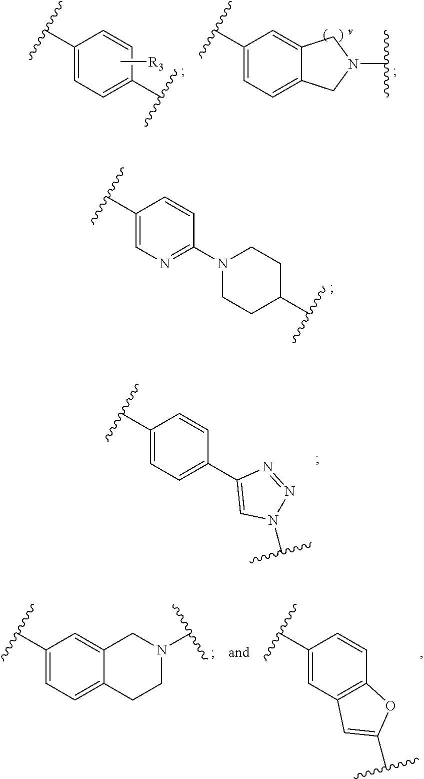

wherein R₃ is selected from hydrogen, C1-C6 alkyl, C1-C6 haloalkyl, C1-C6 alkoxy, halogen, —CN, —NH₂, and —OH, and v is 1 or 2;
L is

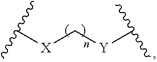

wherein X is selected from —C(═O)—, —CH₂—, C(═O)O—, —C—, —O—, —S—, —NH—, —S(═O)—, —C(═O)NH—, —C(═O)NCH₃—, and

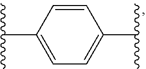

wherein Y is absent or is selected from —O—, —CH₂—, —NH—, —NCH₃—, —[OCH₂CH₂]ₘ—,

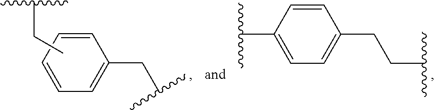

wherein m is an integer selected from 1, 2, 3, 4, and 5, and wherein n is an integer selected from 0, 1, 2, 3, 4, 5, 6, 7, 8, 9, and 10; and
B is selected from hydrogen, —NH₂, —NH(CH₃)—C(═O)(C1-C6 alkyl), —C(═O)(C1-C6 haloalkyl), C1-C6 alkyl, —O(C1-C6 alkyl), —COOH, —OH, —NH(C1-C6 alkyl), —N(C1-C6 alkyl)₂,

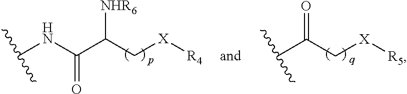

wherein p and q are an integer independently selected from 0, 1, 2, 3, 4, 5, 6, 7, 8, 9, and 10, X is selected from —CH₂—, —NH—, and —O—, R₆ is hydrogen or —C(═O)CH₃—, and R₄ and R₅ are independently selected from hydrogen, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

In some embodiments, R₁ is -cyclopropyl.

In some embodiments, A is selected from

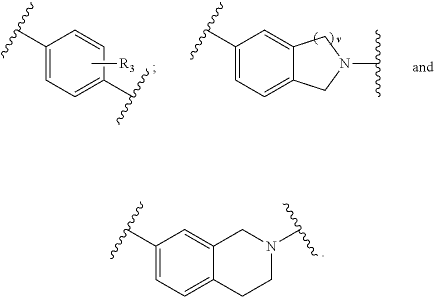

In some embodiments, R₁ is -cyclopropyl and A is selected from

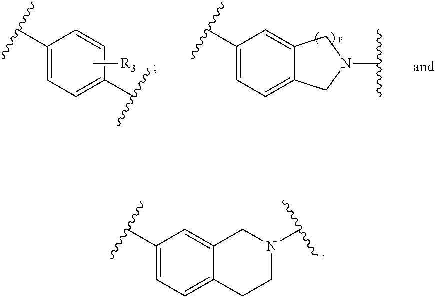

In some embodiments, X is selected from —O—, —CH₂—, —C(═O)NH—, —C(═O)N(CH₃)—, —C(═O), and

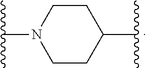 [SMILES: *C1CCN(*)CC1]

In some embodiments, X is selected from —C(═O)NH and —C(═O).

In some embodiments, R₁ is -cyclopropyl; A is selected from

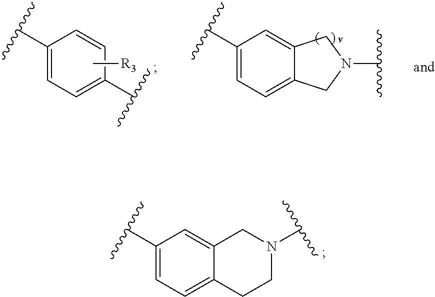

and X is selected from —O—, —CH₂—, —C(═O)NH—, —C(═O)N(CH₃)—, —C(═O), and

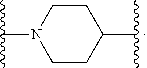 [SMILES: *C1CCN(*)CC1]

In some embodiments, R₁ is -cyclopropyl; A is selected from

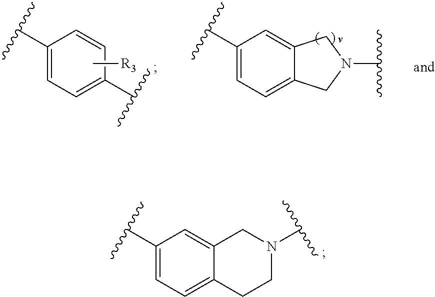

and X is selected from —C(═O)NH and —C(═O).

In some embodiments, R₂ₐ and R_{2b} are independently selected from hydrogen, halogen, and C1-C6 alkoxy.

In some embodiments, R₁ is -cyclopropyl; A is selected from

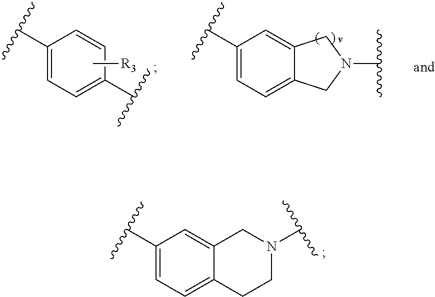

X is selected from —O—, —CH₂—, —C(═O)NH—, —C(═O)N(CH₃)—, —C(═O), and

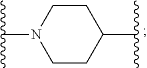 [SMILES: *C1CCN(*)CC1]

and R₂ₐ and R_{2b} are independently selected from hydrogen, halogen, and C1-C6 alkoxy.

In some embodiments, R₁ is -cyclopropyl; A is

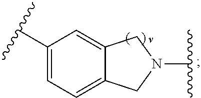

X is selected from —O—, —CH₂—, —C(═O)NH—, —C(═O)N(CH₃)—, —C(═O), and

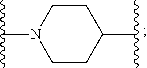 [SMILES: *C1CCN(*)CC1]

and R₂ₐ and R_{2b} are independently selected from hydrogen, halogen, and C1-C6 alkoxy.

In some embodiments, R₁ is -cyclopropyl; A is

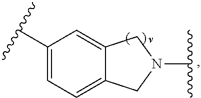

wherein v is 2; X is selected from —O—, —CH₂—, —C(═O)NH—, —C(═O)N(CH₃)—, —C(═O), and

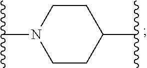 [SMILES: *C1CCN(*)CC1]

and R₂ₐ and R_{2b} are hydrogen.

In some embodiments, when A is

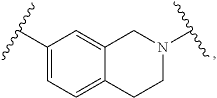 [SMILES: *c1ccc2c(c1)CN(*)CC2]

L cannot be

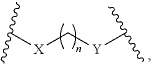

wherein X is —C(═O)NH—, n is 4, Y is —CH₂, and B is —NH₂.

In some embodiments, the compound disclosed herein comprises a compound of Formula (II):

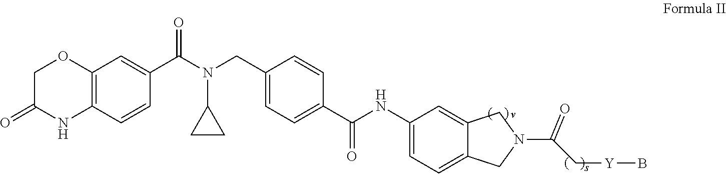

or an enantiomer, an enantiomeric mixture, or a pharmaceutically acceptable salt thereof; wherein:
v is 1 or 2;
s is an integer selected from 1, 2, 3, 4, 5, 6, 7, 8, 9, and 10;
Y is absent or is selected from —O—, —CH₂—, —NH—, —NCH₃—, —[OCH₂CH₂]ₘ—,

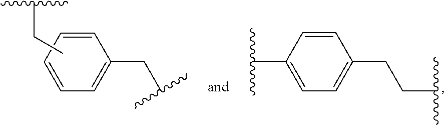

wherein m is an integer selected from 1, 2, 3, 4, and 5; and
B is selected from —H, —NH₂—, —NH(CH₃), —C(═O)(C1-C6 alkyl), —C(═O)(C1-C6 haloalkyl), C1-C6 alkyl, —O(C1-C6 alkyl), —COOH, —OH, —N(C1-C6 alkyl), —N(C1-C6 alkyl)₂

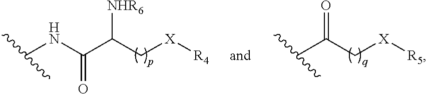

wherein p and q are independently an integer selected from 0, 1, 2, 3, 4, 5, 6, 7, 8, 9, and 10, X is selected from —CH₂—, —NH—, and —O—, R₆ is H or —C(═O)CH₃—, and R₄ and R₅ are independently selected from —H, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

In some embodiments, Y is —CH₂—.

In some embodiments, s is selected from 3, 4 and 5. In some embodiments, Y is —CH₂— and s is selected from 3, 4 and 5.

In some embodiments, B is selected from —NH₂ and

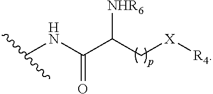

In some embodiments, B is —NH₂. In some embodiments, Y is —CH₂—; s is selected from 3, 4 and 5, and B is —NH₂.

In some embodiments, B is

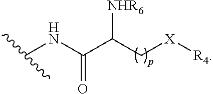

In some embodiments, B is

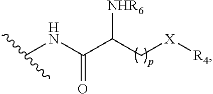

wherein p is an integer selected from 0, 1 and 2. In some embodiments, X is —CH₂—. In some embodiments, R₆ is H. In some embodiments, R₄ is selected from C5-C10 heteroraryl, and —NHC(═NH₂)NH₂. In some embodiments, p is an integer selected from 0, 1 and 2, X is —CH₂—, R₆ is H, and R₄ is selected from C5-C10 heteroraryl, and —NHC(═NH₂)NH₂.

In some embodiments, Y is —CH₂—; s is selected from 3, 4 and 5, and B is

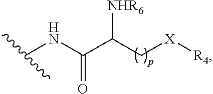

wherein p is an integer selected from 0, 1 and 2, X is —CH₂—, R₆ is H, and R₄ is selected from C5-C10 heteroraryl, and —NHC(═NH₂)NH₂.

In some embodiments, Y is —CH₂—; s is selected from 3, 4 and 5, and B is

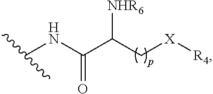

wherein p is an integer selected from 0, 1 and 2, X is —CH₂—, R₆ is H, and R₄ is selected from C5-C10 heteroaryl, and —NHC(═NH₂)NH₂.

In some embodiments, the compound disclosed herein is a compound of Formula (III):

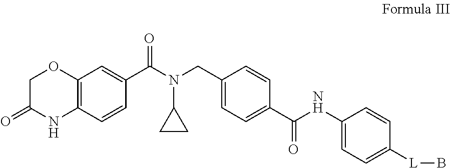

or an enantiomer, an enantiomeric mixture, or a pharmaceutically acceptable salt thereof; wherein:
L is

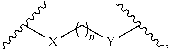

wherein X is selected from —C(═O)—, —CH₂—, C(═O)O—, —C≡C—, —O—, —S—, —NH—, —S(═O)—, —C(═O)NH—, —C(═O)NCH₃—, and

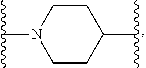

wherein Y is present or absent, and when present is selected from —O—, —CH₂, —NH—, —NCH₃—, —[OCH₂CH₂]ₘ—,

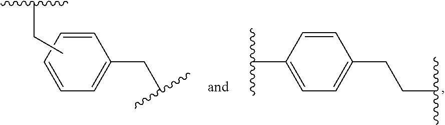

wherein m is an integer selected from 1, 2, 3, 4, and 5, and wherein n is selected from integer 0, 1, 2, 3, 4, 5, 6, 7, 8, 9, and 10; and
B is selected from hydrogen, —NH₂, —NH(CH₃)—C(═O)(C1-C6 alkyl), —C(═O)(C1-C6 haloalkyl), C1-C6 alkyl, —O(C1-C6 alkyl), —COOH, —OH, —NH(C1-C6 alkyl), —N(C1-C6 alkyl)₂,

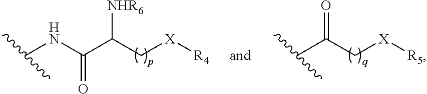

wherein p and q are independently an integer selected from 0, 1, 2, 3, 4, 5, 6, 7, 8, 9, and 10, X is selected from —CH₂—, —NH—, and —O—, R₆ is hydrogen or —C(═O)CH₃—, and R₄ and R₅ is independently selected from hydrogen, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

In some embodiments, L is

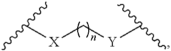

wherein X is selected from —O— and —C(═O)NH—. In some embodiments, n is an integer selected from 5, 6 and 7. In some embodiments, X is selected from —O— and —C(═O)NH—; and n is an integer selected from 5, 6 and 7.

In some embodiments, B is selected from from hydrogen, —OH, —NH₂, —NH(CH₃), and

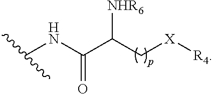

In some embodiments, L is

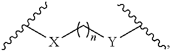

wherein X is selected from —O— and —C(═O)NH—; n is an integer selected from 5, 6 and 7; and B is selected from from hydrogen, —OH, —NH₂, —NH(CH₃), and

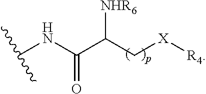

In some embodiments, B is —NH₂.

In some embodiments, L is

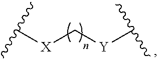

wherein X is —O—; n is an integer selected from 5, 6 and 7; and B is selected from from hydrogen, —NH₂, and

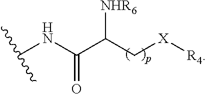

In some embodiments, L is

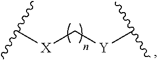

wherein X is —C(═O)NH; n is an integer selected from 5, 6 and 7; and B is selected from from hydrogen, —NH₂, and

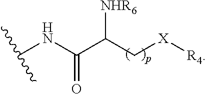

In some embodiments, B is

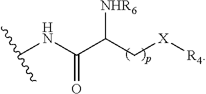

In some embodiments R₆ is H or C(═O)CH₃. In some embodiments, p is an integer selected from 0, 1, 2, and 3. In some embodiments, R₄ is selected from —C1-C6 alkyl, —C1-C6 haloalkyl, C5-C10 aryl, C5-C10 heteroraryl, N(CH₃)C(═NH)NH₂, —N═C(NH₂)NH₂, and —N—C(═NH)NHCH₃. In some embodiments, R₆ is H or C(═O)CH₃; p is an integer selected from 0, 1, 2, and 3; and R₄ is selected from —C1-C6 alkyl, —C1-C6 haloalkyl, C5-C10 aryl, C5-C10 heteroraryl, N(CH₃)C(═NH)NH₂, —N═C(NH₂)NH₂, and —N—C(═NH)NHCH₃. In some embodiments, R₆ is H, p is 3, and R₄ is selected from C5-C10 aryl, C5-C10 heteroraryl, N(CH₃)C(═NH)NH₂, —N═C(NH₂)NH₂, and —N—C(═NH)NHCH₃. In some embodiments, R₆ is H, p is 3, and R₄ is —N═C(NH₂)NH₂.

A compound of any one of Formula (I), (II), and (III) may be selected from the compound listed in Table 1. Compounds of Formula (I), (II), and (III) that are not listed in Table 1 are also within the scope herein.

TABLE 1

| Compound | | |
|---|---|---|
| No. | Structures | MW* |
| 1 | 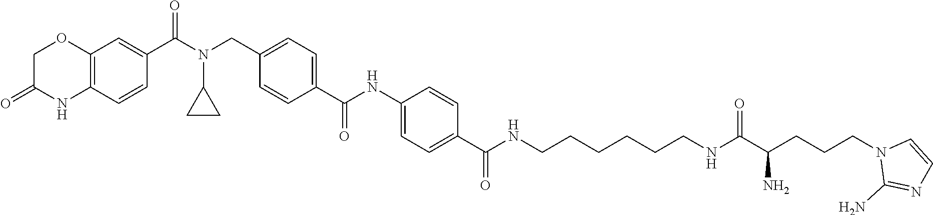 [SMILES: Nc1nccn1CCC[C@@H](N)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(C1═CC═C(C═C1)CN(C2CC2)C(C3═CC(OC4)═C(C═C3)NC4═O)═O)NC5═CC═C(C═C5)C(NCCCCCCNC([C@H](N)CCCN6C═CN═C6N)═O)═O | |
| 2 | 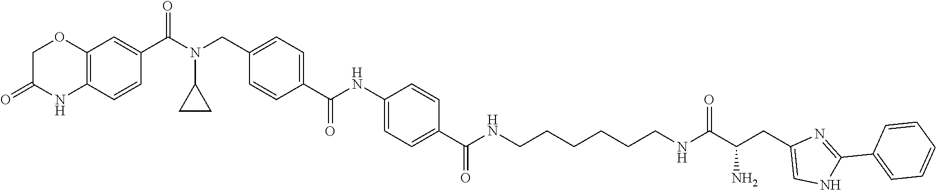 [SMILES: N[C@@H](Cc1c[nH]c(-c2ccccc2)n1)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(NC1═CC═C(C(NCCCCCCNC([C@@H](N)CC2═CNC(C3═CC═CC═C3)═N2)═O)═O)C═C1)C4═CC═C(CN(C(C5═CC(OCC(N6)═O)═C6C═C5)═O)C7CC7)C═C4 | |
| 3 | 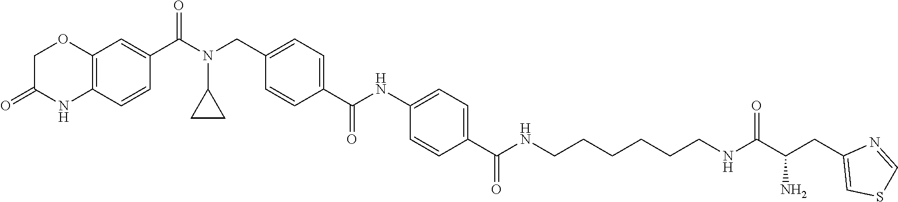 [SMILES: N[C@@H](Cc1cscn1)C(=O)NCCCCCCNC(=O)c1ccc(NC(=O)c2ccc(CN(C(=O)c3ccc4c(c3)OCC(=O)N4)C3CC3)cc2)cc1] | 851.89 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CC6═CSC═N6)═O | |
| 4 | 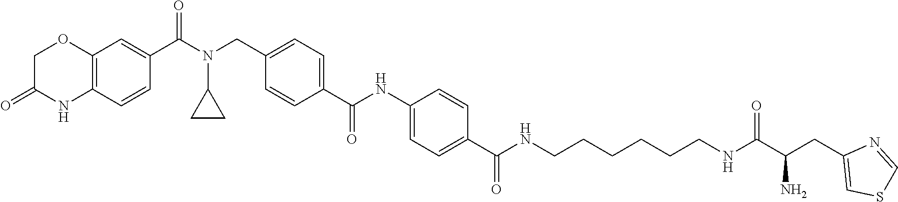 [SMILES: N[C@H](Cc1cscn1)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(C1═CC═C(C═C1)CN(C2CC2)C(C3═CC(OC4)═C(C═C3)NC4-O)═O)NC5═CC═C(C═C5)C(NCCCCCCNC([C@@H](CC6═CSC═N6)N)═O)═O | |
| 5 | 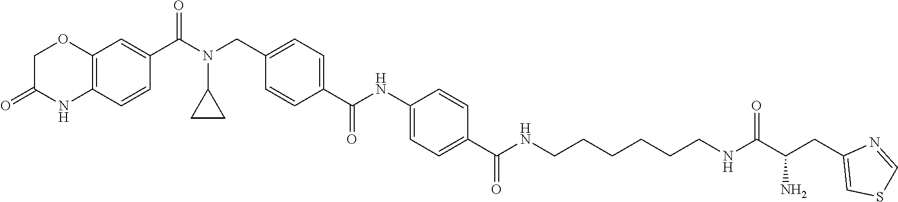 [SMILES: N[C@@H](Cc1cscn1)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CSC═N6)N)═O)═O | |
| 6 | 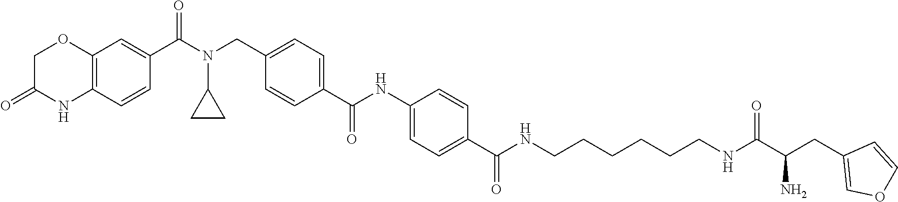 [SMILES: N[C@H](Cc1ccoc1)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(C1═CC═C(C═C1)CN(C2CC2)C(C3═CC(OC4)═C(C═C3)NC4═O)═O)NC5═CC═C(C═C5)C(NCCCCCCNC([C@@H](CC6═COC═C6)N)═O)═O | |
| 7 | 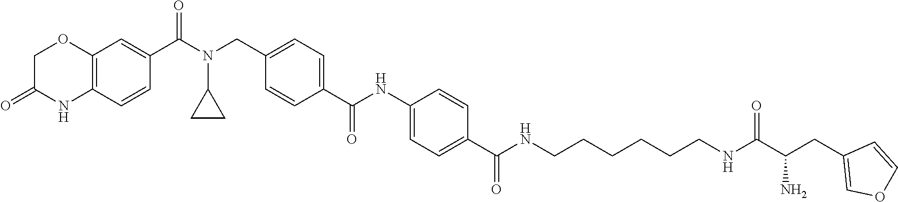 [SMILES: N[C@@H](Cc1ccoc1)C(=O)NCCCCCCNC(=O)c1ccc(NC(=O)c2ccc(CN(C(=O)c3ccc4c(c3)OCC(=O)N4)C3CC3)cc2)cc1] | NA |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═COC═C6)N)═O)═O | |
| 8 | 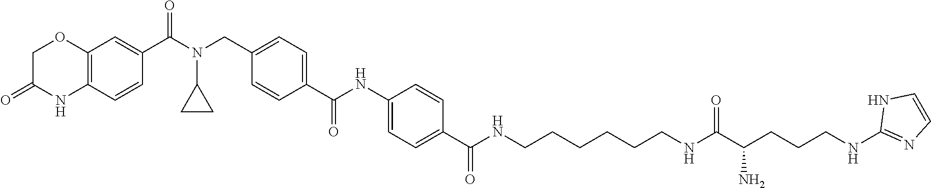 [SMILES: N[C@@H](CCCNc1ncc[nH]1)C(=O)NCCCCCCNC(=O)c1ccc(NC(=O)c2ccc(CN(C(=O)c3ccc4c(c3)OCC(=O)N4)C3CC3)cc2)cc1] | 836.81 |
| | O═C(NC1═CC═C(C(NCCCCCCNC([C@@H](N)CCCNC2═NC═CN2)═O)═O)C═C1)C3═CC═C(CN(C(C4═CC(OCC(N5)═O)═C5C═C4)═O)C6CC6)C═C3 | |
| 9 | 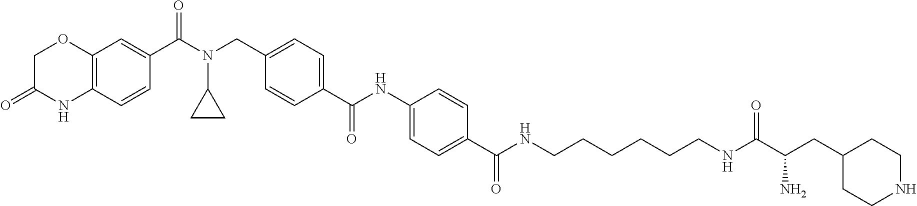 [SMILES: N[C@@H](CC1CCNCC1)C(=O)NCCCCCCNC(=O)c1ccc(NC(=O)c2ccc(CN(C(=O)c3ccc4c(c3)OCC(=O)N4)C3CC3)cc2)cc1] | 810.81 |
| | O═C(NC1═CC═C(C(NCCCCCCNC([C@@H](N)CC2CCNCC2)═O)═O)C═C1)C3═CC═C(CN(C(C4═CC(OCC(N5)═O)═C5C═C4)═O)C6CC6)C═C3 | |
| 10 | 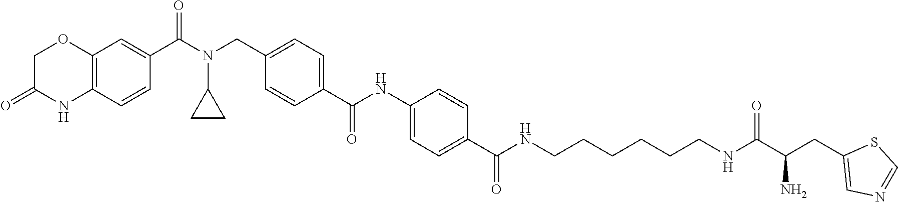 [SMILES: N[C@H](Cc1cncs1)C(=O)NCCCCCCNC(=O)c1ccc(NC(=O)c2ccc(CN(C(=O)c3ccc4c(c3)OCC(=O)N4)C3CC3)cc2)cc1] | 774.33 |
| | O═C(C1═CC═C(C═C1)CN(C2CC2)C(C3═CC(OC4)═C(C═C3)NC4═O)═O)NC5═CC═C(C═C5)C(NCCCCCCNC([C@@H](CC6═CN═CS6)N)═O)═O | |
| 11 | 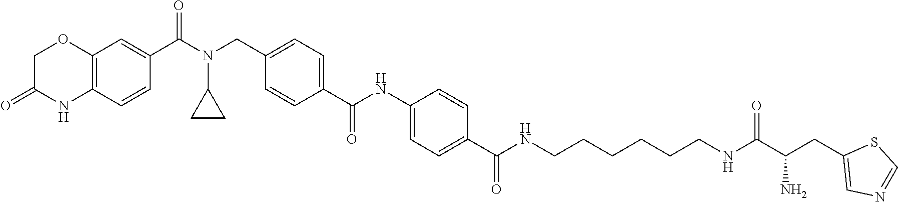 [SMILES: N[C@@H](Cc1cncs1)C(=O)NCCCCCCNC(=O)c1ccc(NC(=O)c2ccc(CN(C(=O)c3ccc4c(c3)OCC(=O)N4)C3CC3)cc2)cc1] | 774.33 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CN═CS6)N)═O)═O | |
| 12 | 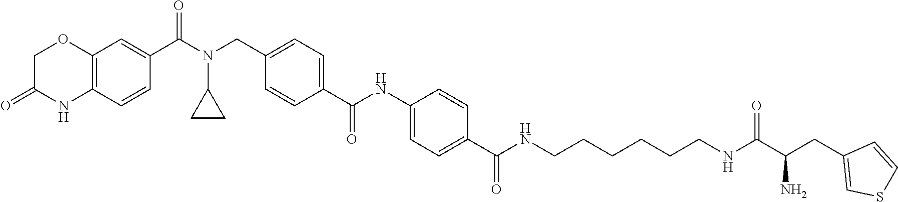 [SMILES: N[C@H](Cc1ccsc1)C(=O)NCCCCCCNC(=O)c1ccc(NC(=O)c2ccc(CN(C(=O)c3ccc4c(c3)OCC(=O)N4)C3CC3)cc2)cc1] | 773.34 |
| | O═C(C1═CC═C(C═C1)CN(C2CC2)C(C3═CC(OC4)═C(C═C3)NC4═O)═O)NC5═CC═C(C═C5)C(NCCCCCCNC([C@@H](CC6═CSC═C6)N)═O)═O | |
| 13 | 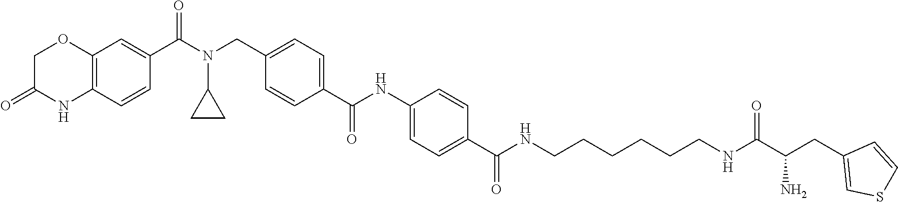 [SMILES: N[C@@H](Cc1ccsc1)C(=O)NCCCCCCNC(=O)c1ccc(NC(=O)c2ccc(CN(C(=O)c3ccc4c(c3)OCC(=O)N4)C3CC3)cc2)cc1] | 773.34 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CSC═C6)N)═O)═O | |
| 14 | 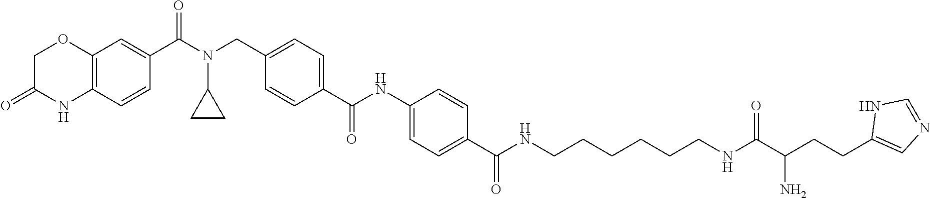 [SMILES: NC(CCc1cnc[nH]1)C(=O)NCCCCCCNC(=O)c1ccc(NC(=O)c2ccc(CN(C(=O)c3ccc4c(c3)OCC(=O)N4)C3CC3)cc2)cc1] | 807.77 |
| | O═C(NC1═CC═C(C(NCCCCCCNC(C(N)CCC2═CN═CN2)═O)═O)C═C1)C3═CC═C(CN(C(C4═CC(OCC(N5)═O)═C5C═C4)═O)C6CC6)C═C3 | |
| 15 | 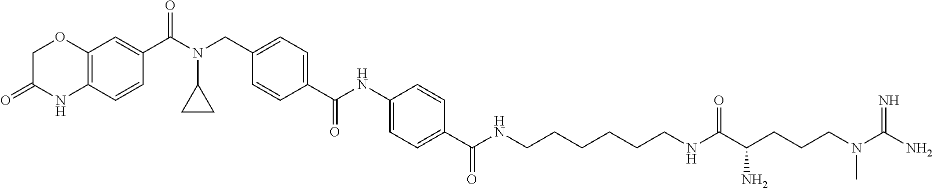 [SMILES: CN(CCC[C@H](N)C(=O)NCCCCCCNC(=O)c1ccc(NC(=O)c2ccc(CN(C(=O)c3ccc4c(c3)OCC(=O)N4)C3CC3)cc2)cc1)C(=N)N] | 826.81 |
| | O═C(NC1═CC═C(C(NCCCCCCNC([C@@H](N)CCCN(C)C(N)═N)═O)═O)C═C1)C2═CC═C(CN(C(C3═CC(OCC(N4)═O)═C4C═C3)═O)C5CC5)C═C2 | |
| 16 | 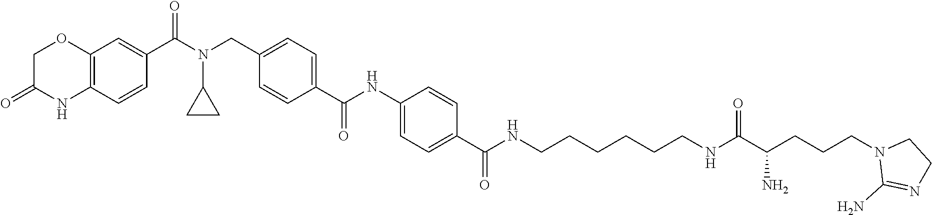 [SMILES: NC1=NCCN1CCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 838.82 |
| | O═C(NC1═CC═C(C(NCCCCCCNC([C@@H](N)CCCN2CCN═C2N)═O)═O)C═C1)C3═CC═C(CN(C(C4═CC(OCC(N5)═O)═C5C═C4)═O)C6CC6)C═C3 | |
| 17 | 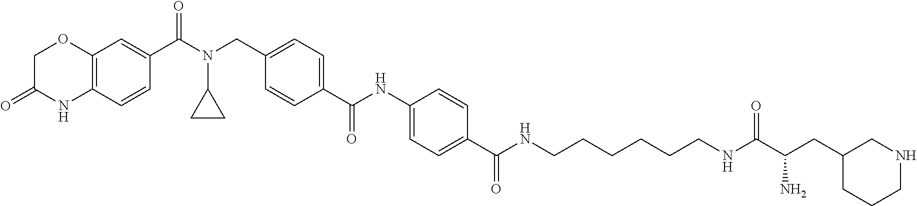 [SMILES: N[C@@H](CC1CCCNC1)C(=O)NCCCCCCNC(=O)c1ccc(NC(=O)c2ccc(CN(C(=O)c3ccc4c(c3)OCC(=O)N4)C3CC3)cc2)cc1] | 810.81 |
| | O═C(NC1═CC═C(C(NCCCCCCNC([C@@H](N)CC2CCCNC2)═O)═O)C═C1)C3═CC═C(CN(C(C4═CC(OCC(N5)═O)═C5C═C4)═O)C6CC6)C═C3 | |
| 18 | 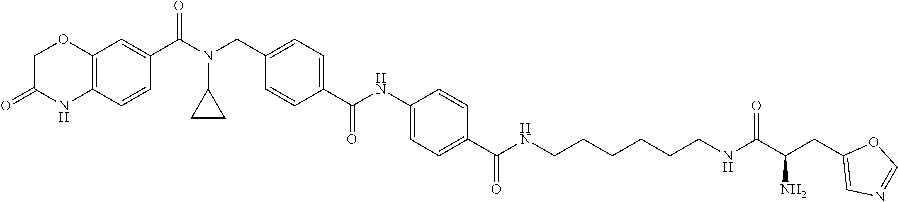 [SMILES: N[C@H](Cc1cnco1)C(=O)NCCCCCCNC(=O)c1ccc(NC(=O)c2ccc(CN(C(=O)c3ccc4c(c3)OCC(=O)N4)C3CC3)cc2)cc1] | 758.26 |
| | O═C(C1═CC═C(C═C1)CN(C2CC2)C(C3═CC(OC4)═C(C═C3)NC4═O)═O)NC5═CC═C(C═C5)C(NCCCCCCNC([C@@H](CC6═CN═CO6)N)═O)═O | |
| 19 | 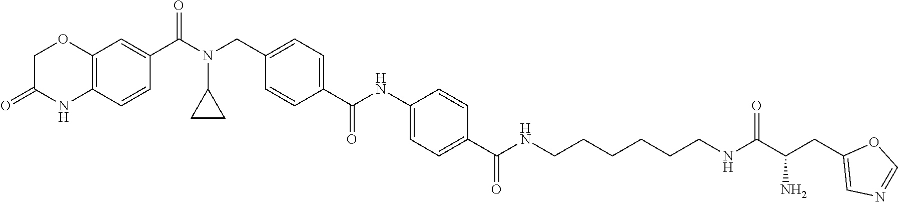 [SMILES: N[C@@H](Cc1cnco1)C(=O)NCCCCCCNC(=O)c1ccc(NC(=O)c2ccc(CN(C(=O)c3ccc4c(c3)OCC(=O)N4)C3CC3)cc2)cc1] | 758.26 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CN═CO6)N)═O)═O | |
| 20 | 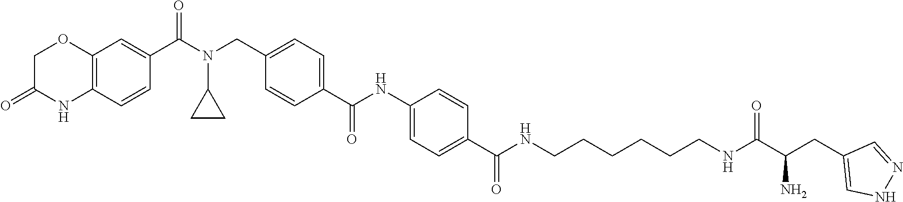 [SMILES: N[C@H](Cc1cn[nH]c1)C(=O)NCCCCCCNC(=O)c1ccc(NC(=O)c2ccc(CN(C(=O)c3ccc4c(c3)OCC(=O)N4)C3CC3)cc2)cc1] | 793.74 |
| | O═C(C1═CC═C(C═C1)CN(C2CC2)C(C3═CC(OC4)═C(C═C3)NC4═O)═O)NC5═CC═C(C═C5)C(NCCCCCCNC([C@@H](CC6═CNN═C6)N)═O)═O | |
| 21 | 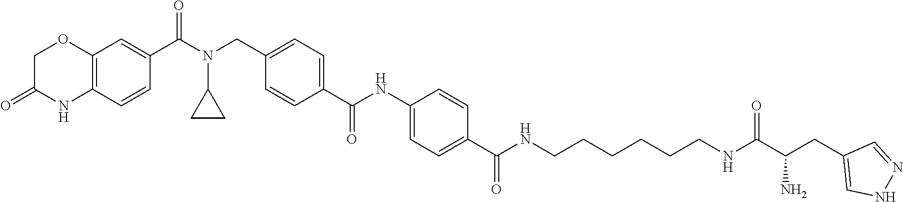 [SMILES: N[C@@H](Cc1cn[nH]c1)C(=O)NCCCCCCNC(=O)c1ccc(NC(=O)c2ccc(CN(C(=O)c3ccc4c(c3)OCC(=O)N4)C3CC3)cc2)cc1] | 793.74 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CNN═C6)N)═O)═O | |
| 22 | 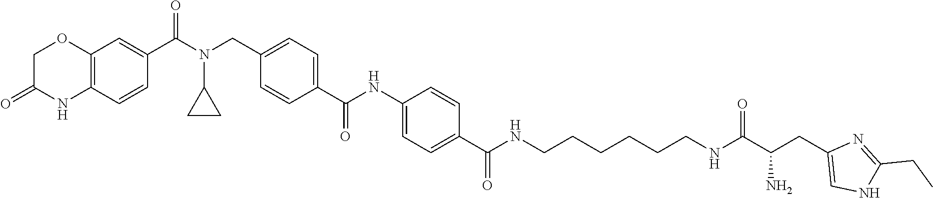 [SMILES: CCc1nc(C[C@H](N)C(=O)NCCCCCCNC(=O)c2ccc(NC(=O)c3ccc(CN(C(=O)c4ccc5c(c4)OCC(=O)N5)C4CC4)cc3)cc2)c[nH]1] | 785.33 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CNC(CC)═N6)N)═O)═O | |
| 23 | 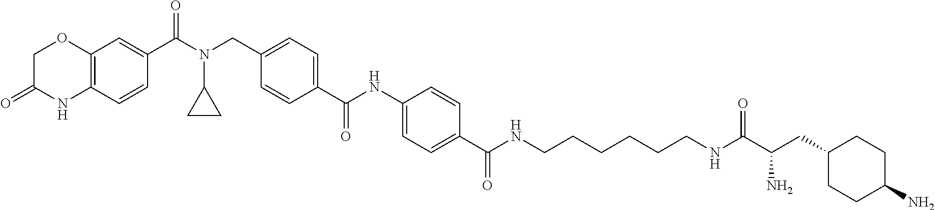 [SMILES: N[C@@H](C[C@H]1CC[C@H](N)CC1)C(=O)NCCCCCCNC(=O)c1ccc(NC(=O)c2ccc(CN(C(=O)c3ccc4c(c3)OCC(=O)N4)C3CC3)cc2)cc1] | 788.37 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](C[C@H]6CC[C@@H](CC6)N)N)═O)═O | |
| 24 | 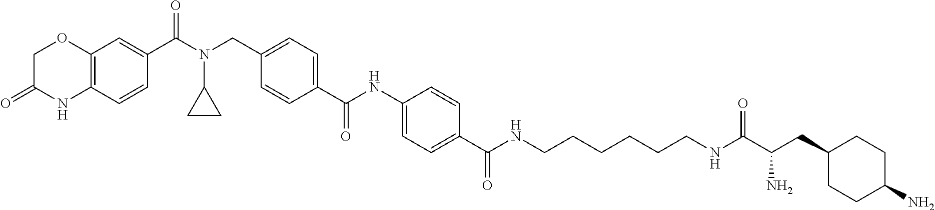 [SMILES: N[C@@H](C[C@H]1CC[C@@H](N)CC1)C(=O)NCCCCCCNC(=O)c1ccc(NC(=O)c2ccc(CN(C(=O)c3ccc4c(c3)OCC(=O)N4)C3CC3)cc2)cc1] | 788.37 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](C[C@@H]6CC[C@@H](CC6)N)N)═O)═O | |
| 25 | 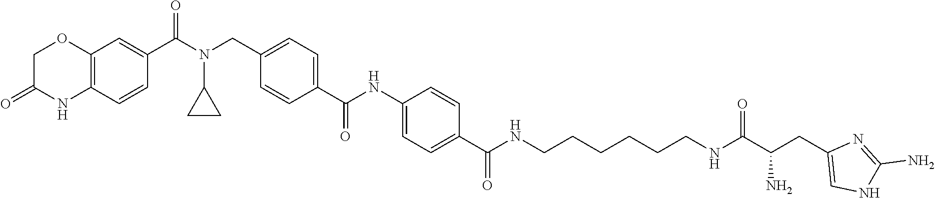 [SMILES: Nc1nc(C[C@H](N)C(=O)NCCCCCCNC(=O)c2ccc(NC(=O)c3ccc(CN(C(=O)c4ccc5c(c4)OCC(=O)N5)C4CC4)cc3)cc2)c[nH]1] | 772.29 |
| | O═C(C1═CC═C(C═C1)CN(C2CC2)C(C3═CC(OC4)═C(C═C3)NC4═O)═O)NC5═CC═C(C═C5)C(NCCCCCCNC([C@H](CC6═CNC(N)═N6)N)═O)═O | |
| 26 | 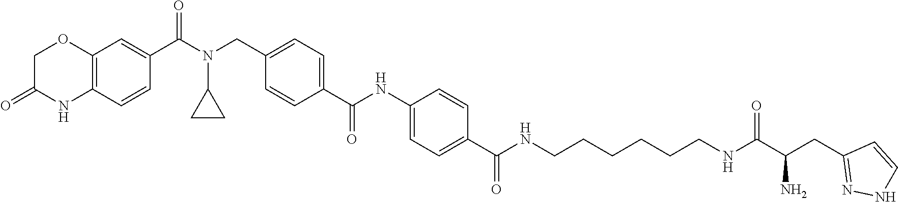 [SMILES: N[C@H](Cc1cc[nH]n1)C(=O)NCCCCCCNC(=O)c1ccc(NC(=O)c2ccc(CN(C(=O)c3ccc4c(c3)OCC(=O)N4)C3CC3)cc2)cc1] | 757.28 |
| | O═C(NC1═CC═C(C(NCCCCCCNC([C@H](N)CC2═NNC═C2)═O)═O)C═C1)C3═CC═C(CN(C(C4═CC(OCC5═O)═C(N5)C═C4)═O)C6CC6)C═C3 | |
| 27 | 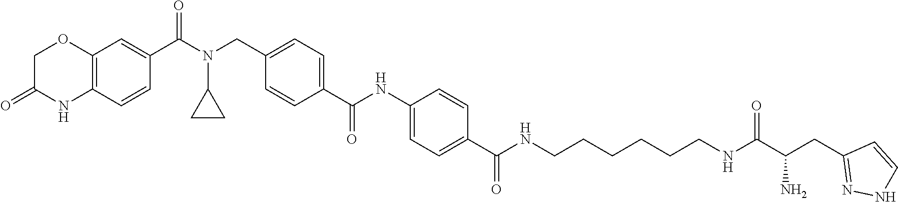 [SMILES: N[C@@H](Cc1cc[nH]n1)C(=O)NCCCCCCNC(=O)c1ccc(NC(=O)c2ccc(CN(C(=O)c3ccc4c(c3)OCC(=O)N4)C3CC3)cc2)cc1] | 757.28 |
| | O═C(NC1═CC═C(C(NCCCCCCNC([C@@H](N)CC2═NNC═C2)═O)═O)C═C1)C3═CC═C(CN(C(C4═CC(OCC5═O)═C(N5)C═C4)═O)C6CC6)C═C3 | |
| 28 | 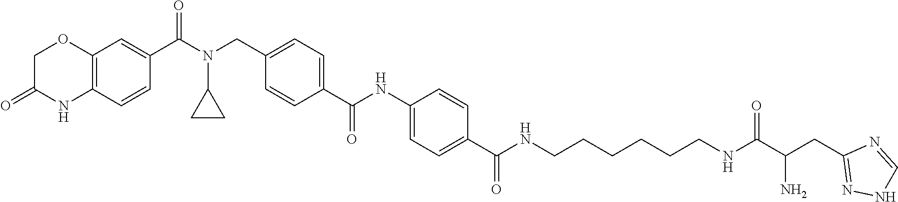 [SMILES: NC(Cc1nc[nH]n1)C(=O)NCCCCCCNC(=O)c1ccc(NC(=O)c2ccc(CN(C(=O)c3ccc4c(c3)OCC(=O)N4)C3CC3)cc2)cc1] | 758.27 |
| | O═C(C1═CC═C(C═C1)CN(C2CC2)C(C3═CC(OC4)═C(C═C3)NC4═O)═O)NC5═CC═C(C═C5)C(NCCCCCCNC(C(CC6═NNC═N6)N)═O)═O | |
| 29 | 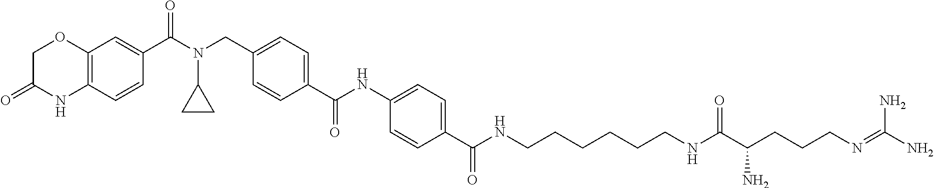 [SMILES: NC(N)=NCCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 967.91 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](CCC/N═C(N)/N)N)═O | |
| 30 | 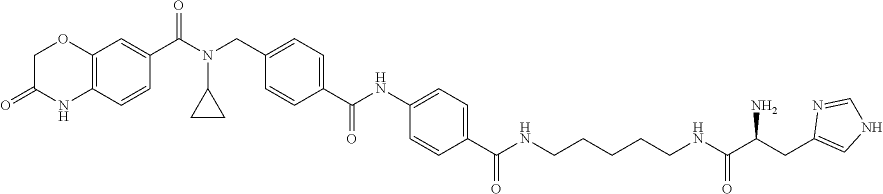 [SMILES: N[C@@H](Cc1c[nH]cn1)C(=O)NCCCCCNC(=O)c1ccc(NC(=O)c2ccc(CN(C(=O)c3ccc4c(c3)OCC(=O)N4)C3CC3)cc2)cc1] | 934.84 |
| | O═C(NCCCCCNC([C@H](CC1═CNC═N1)N)═O)C2═CC═C(NC(C3═CC═C(CN(C4CC4)C(C5═CC(OCC( | |
| | N6)═O)═C6C═C5)═O)C═C3)═O)C═C2 | |
| 31 | 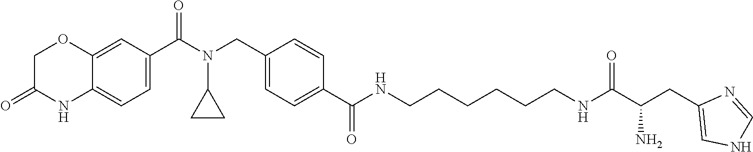 [SMILES: N[C@@H](Cc1c[nH]cn1)C(=O)NCCCCCCNC(=O)c1ccc(CN(C(=O)c2ccc3c(c2)OCC(=O)N3)C2CC2)cc1] | 829.74 |
| | O═C(N(C1CC1)CC2═CC═C(C(NCCCCCCNC([C@H](CC3═CNC═N3)N)═O)═O)C═C2)C4═CC═C5C(OCC(N5)═O)═C4 | |
| 32 | 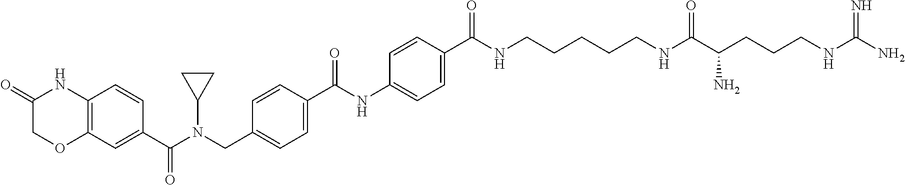 [SMILES: N=C(N)NCCC[C@H](N)C(=O)NCCCCCNC(=O)c1ccc(NC(=O)c2ccc(CN(C(=O)c3ccc4c(c3)OCC(=O)N4)C3CC3)cc2)cc1] | 953.88 |
| | O═C(NCCCCCNC[C@@H](N)CCCNC(N)═N)═O)C1═CC═C(NC(C2═CC═C(CN(C3CC3)C(C4═CC(OCC(N5)═O)═C5C═C4)═O)C═C2)═O)C═C1 | |
| 33 | 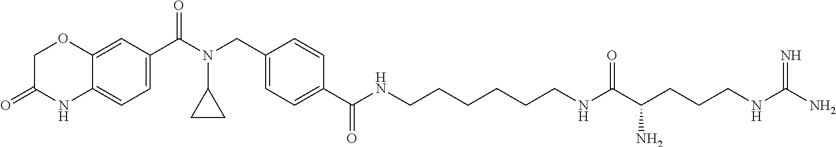 [SMILES: N=C(N)NCCC[C@H](N)C(=O)NCCCCCCNC(=O)c1ccc(CN(C(=O)c2ccc3c(c2)OCC(=O)N3)C2CC2)cc1] | 848.79 |
| | O═C(N(C1CC1)CC2═CC═C(C(NCCCCCCNC([C@@H](N)CCCNC(N)═N)═O)═O)C═C2)C3═CC═C4C(OCC(N4)═O)═C3 | |
| 34 | 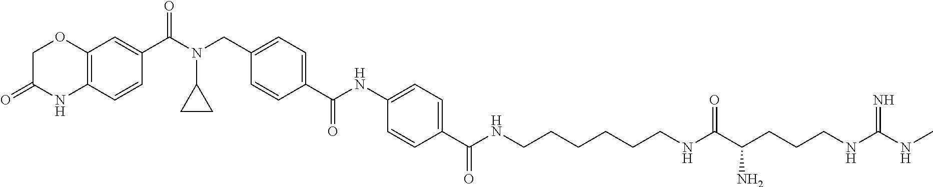 [SMILES: CNC(=N)NCCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 981.94 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](CCCNC(NC)═N)N)═O | |
| 35 | 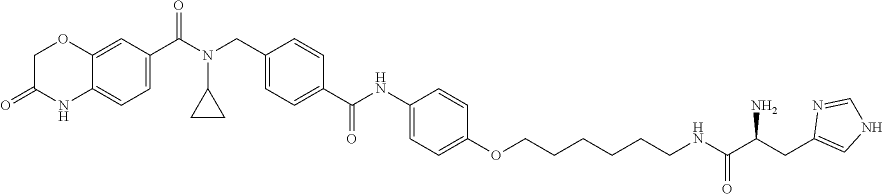 [SMILES: N[C@@H](Cc1c[nH]cn1)C(=O)NCCCCCCOc1ccc(NC(=O)c2ccc(CN(C(=O)c3ccc4c(c3)OCC(=O)N4)C3CC3)cc2)cc1] | 921.84 |
| | O═C(N(C1CC1)CC2═CC═C(C(NC3═CC═C(OCCCCCCNC([C@H](CC4═CNC═N4)N)═O)C═C3)═O)C═C2)C5═CC═C6C(OCC(N6)═O)═C5 | |
| 36 | 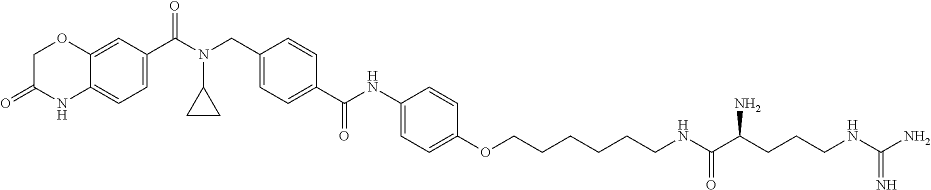 [SMILES: N=C(N)NCCC[C@H](N)C(=O)NCCCCCCOc1ccc(NC(=O)c2ccc(CN(C(=O)c3ccc4c(c3)OCC(=O)N4)C3CC3)cc2)cc1] | 940.88 |
| | O═C(N(C1CC1)CC2═CC═C(C(NC3═CC═C(OCCCCCCNC([C@@H](N)CCCNC(N)═N)═O)C═C3)═O)C═C2)C4═CC═C5C(OCC(N5)═O)═C4 | |
| 37 | 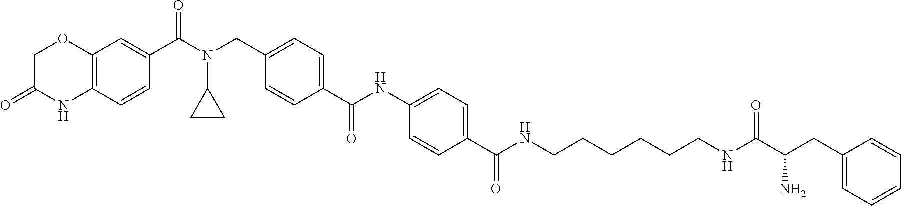 [SMILES: N[C@@H](Cc1ccccc1)C(=O)NCCCCCCNC(=O)c1ccc(NC(=O)c2ccc(CN(C(=O)c3ccc4c(c3)OCC(=O)N4)C3CC3)cc2)cc1] | 844.87 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CC6═CC═CC═C6)═O | |
| 38 | 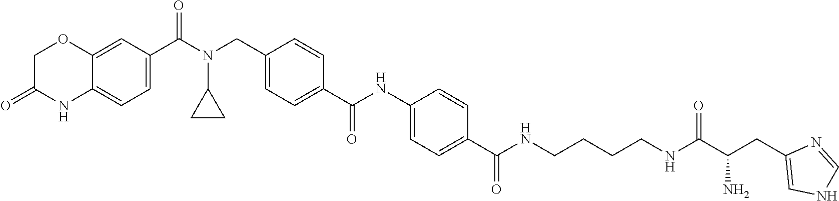 [SMILES: N[C@@H](Cc1c[nH]cn1)C(=O)NCCCCNC(=O)c1ccc(NC(=O)c2ccc(CN(C(=O)c3ccc4c(c3)OCC(=O)N4)C3CC3)cc2)cc1] | 920.81 |
| | O═C(N(C1CC1)CC2═CC═C(C(NC3═CC═C(C(NCCCCNC([C@H](CC4═CNC═N4)N)═O)═O)C═C3)═O)C═C2)C5═CC═C6C(OCC(N6)═O)═C5 | |
| 39 | 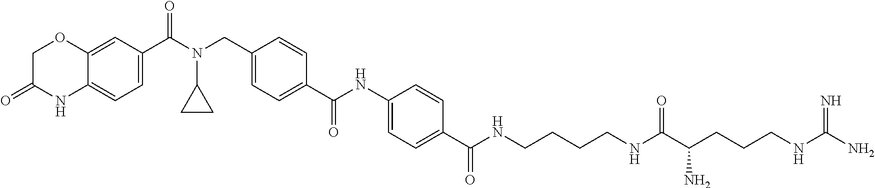 [SMILES: N=C(N)NCCC[C@H](N)C(=O)NCCCCNC(=O)c1ccc(NC(=O)c2ccc(CN(C(=O)c3ccc4c(c3)OCC(=O)N4)C3CC3)cc2)cc1] | 939.86 |
| | O═C(N(C1CC1)CC2═CC═C(C(NC3═CC═C(C(NCCCCNC([C@@H](N)CCCNC(N)═N)═O)═O)C═C3)═O)C═C2)C4═CC═C5C(OCC(N5)═O)═C4 | |
| 40 | 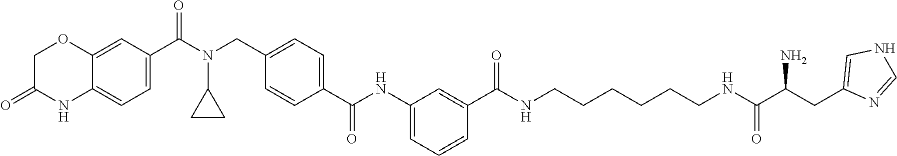 [SMILES: N[C@@H](Cc1c[nH]cn1)C(=O)NCCCCCCNC(=O)c1cccc(NC(=O)c2ccc(CN(C(=O)c3ccc4c(c3)OCC(=O)N4)C3CC3)cc2)c1] | 948.86 |
| | O═C(N(C1CC1)CC2═CC═C(C(NC3═CC═CC(C(NCCCCCCNC([C@H](CC4═CNC═N4)N)═O)═O)═C3)═O)C═C2)C5═CC═C6C(OCC(N6)═O)═C5 | |
| 41 | 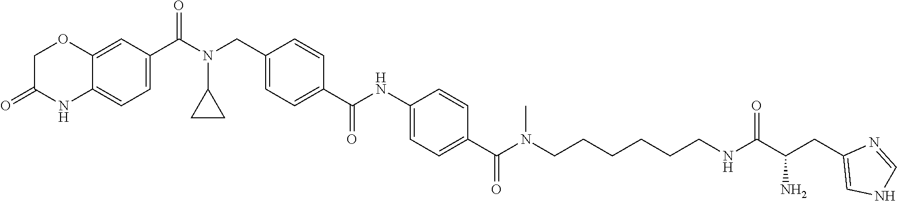 [SMILES: CN(CCCCCCNC(=O)[C@@H](N)Cc1c[nH]cn1)C(=O)c1ccc(NC(=O)c2ccc(CN(C(=O)c3ccc4c(c3)OCC(=O)N4)C3CC3)cc2)cc1] | 962.89 |
| | O═C(N(C1CC1)CC2═CC═C(C(NC3═CC═C(C(N(C)CCCCCCNC([C@H](CC4═CNC═N4)N)═O)═O)C═C3)═O)C═C2)C5═CC═C6C(OCC(N6)═O)═C5 | |
| 42 | 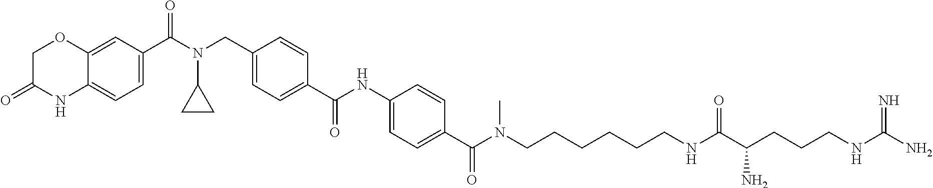 [SMILES: CN(CCCCCCNC(=O)[C@@H](N)CCCNC(=N)N)C(=O)c1ccc(NC(=O)c2ccc(CN(C(=O)c3ccc4c(c3)OCC(=O)N4)C3CC3)cc2)cc1] | 981.94 |
| | O═C(N(C1CC1)CC2═CC═C(C(NC3═CC═C(C(N(C)CCCCCCNC([C@@H](N)CCCNC(N)═N)═O)═O)C═C3)═O)C═C2)C4═CC═C5C(OCC(N5)═O)═C4 | |
| 43 | 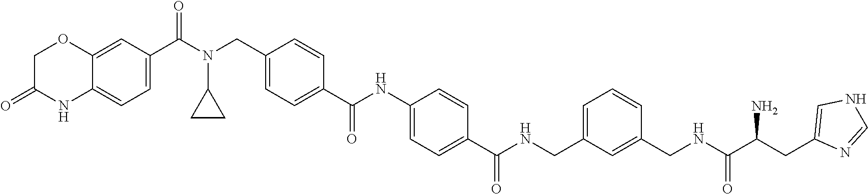 [SMILES: N[C@@H](Cc1c[nH]cn1)C(=O)NCc1cccc(CNC(=O)c2ccc(NC(=O)c3ccc(CN(C(=O)c4ccc5c(c4)OCC(=O)N5)C4CC4)cc3)cc2)c1] | 968.85 |
| | N[C@@H](CC1═CNC═N1)C(NCC2═CC(CNC(C3═CC═C(NC(C4═CC═C(CN(C(C5═CC═C6NC(COC6═C5)═O)═O)C7CC7)C═C4)═O)C═C3)═O)═CC═C2)═O | |
| 44 | 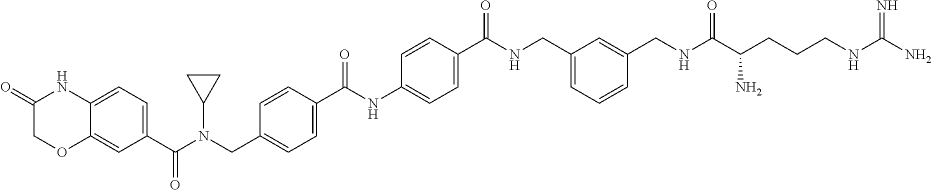 [SMILES: N=C(N)NCCC[C@H](N)C(=O)NCc1cccc(CNC(=O)c2ccc(NC(=O)c3ccc(CN(C(=O)c4ccc5c(c4)OCC(=O)N5)C4CC4)cc3)cc2)c1] | 987.9 |
| | N═C(NCCC[C@@H](C(NCC1═CC(CNC(C2═CC═C(NC(C3═CC═C(CN(C(C4═CC═C5NC(COC5═C4)═O)═O)C6CC6)C═C3)═O)C═C2)═O)═CC═C1)═O)N)N | |
| 45 | 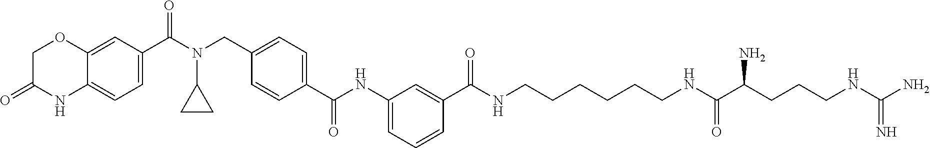 [SMILES: N=C(N)NCCC[C@H](N)C(=O)NCCCCCCNC(=O)c1cccc(NC(=O)c2ccc(CN(C(=O)c3ccc4c(c3)OCC(=O)N4)C3CC3)cc2)c1] | 967.91 |
| | O═C(N(C1CC1)CC2═CC═C(C(NC3═CC═CC(C(NCCCCCCNC([C@@H](N)CCCNC(N)═N)═O)═O)═C3)═O)C═C2)C4═CC═C5C(OCC(N5)═O)═C4 | |
| 46 | 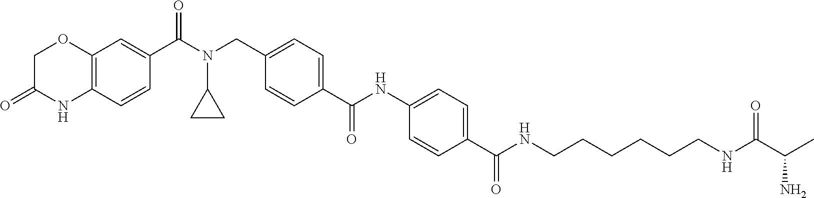 [SMILES: C[C@H](N)C(=O)NCCCCCCNC(=O)c1ccc(NC(=O)c2ccc(CN(C(=O)c3ccc4c(c3)OCC(=O)N4)C3CC3)cc2)cc1] | 768.78 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)C)═O | |
| 47 | 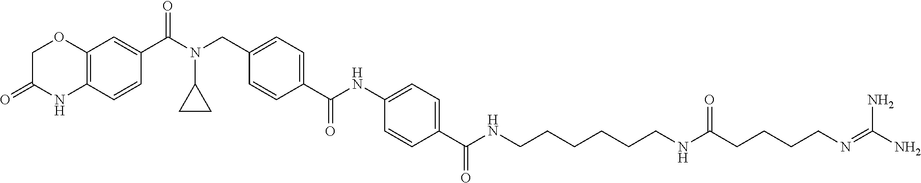 [SMILES: NC(N)=NCCCCC(=O)NCCCCCCNC(=O)c1ccc(NC(=O)c2ccc(CN(C(=O)c3ccc4c(c3)OCC(=O)N4)C3CC3)cc2)cc1] | 838.87 |
| | O═C(C1═CC═C(NC(C2═CC═C(CN(C3CC3)C(C4═CC(OCC(N5)═O)═C5C═C4)═O)C═C2)═O)C═C1)NCCCCCCNC(CCCC/N═C(N)/N)═O | |
| 48 | 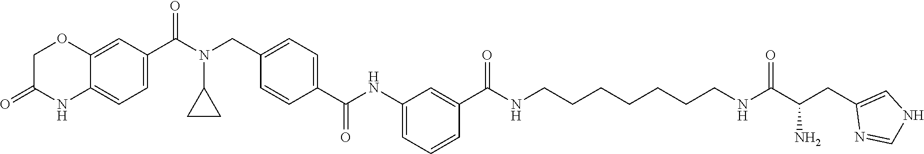 [SMILES: N[C@@H](Cc1c[nH]cn1)C(=O)NCCCCCCCNC(=O)c1cccc(NC(=O)c2ccc(CN(C(=O)c3ccc4c(c3)OCC(=O)N4)C3CC3)cc2)c1] | 962.89 |
| | O═C(N(C1CC1)CC2═CC═C(C(NC3═CC═CC(C(NCCCCCCCNC([C@H](CC4═CNC═N4)N)═O)═O)═C3)═O)C═C2)C5═CC═C6C(OCC(N6)═O)═C5 | |
| 49 | 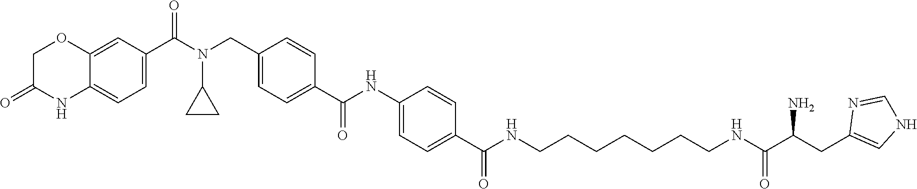 [SMILES: N[C@@H](Cc1c[nH]cn1)C(=O)NCCCCCCCNC(=O)c1ccc(NC(=O)c2ccc(CN(C(=O)c3ccc4c(c3)OCC(=O)N4)C3CC3)cc2)cc1] | 962.89 |
| | O═C(N(C1CC1)CC2═CC═C(C(NC3═CC═C(C(NCCCCCCCNC([C@H](CC4═CNC═N4)N)═O)═O)C═C3)═O)C═C2)C5═CC═C6C(OCC(N6)═O)═C5 | |
| 50 | 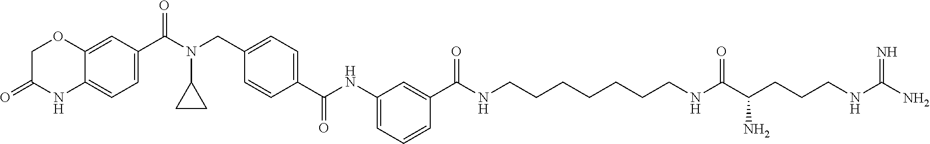 [SMILES: N=C(N)NCCC[C@H](N)C(=O)NCCCCCCCNC(=O)c1cccc(NC(=O)c2ccc(CN(C(=O)c3ccc4c(c3)OCC(=O)N4)C3CC3)cc2)c1] | 981.94 |
| | O═C(N(C1CC1)CC2═CC═C(C(NC3═CC═CC(C(NCCCCCCCNC([C@@H](N)CCCNC(N)═N)═O)═O)═C3)═O)C═C2)C4═CC═C5C(OCC(N5)═O)═C4 | |
| 51 | 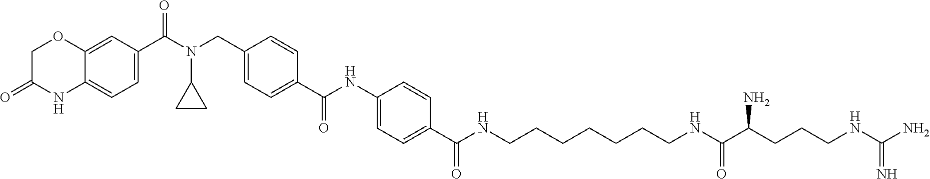 [SMILES: N=C(N)NCCC[C@H](N)C(=O)NCCCCCCCNC(=O)c1ccc(NC(=O)c2ccc(CN(C(=O)c3ccc4c(c3)OCC(=O)N4)C3CC3)cc2)cc1] | 981.94 |
| | O═C(N(C1CC1)CC2═CC═C(C(NC3═CC═C(C(NCCCCCCCNC([C@@H](N)CCCNC(N)═N)═O)═O)C═C3)═O)C═C2)C4═CC═C5C(OCC(N5)═O)═C4 | |
| 52 | 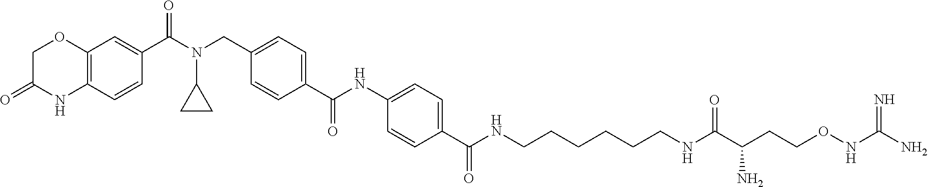 [SMILES: N=C(N)NOCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 969.88 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CCONC(N)═N)═O | |
| 53 | 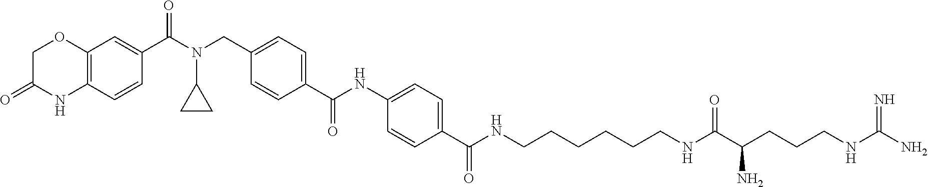 [SMILES: N=C(N)NCCC[C@@H](N)C(=O)NCCCCCCNC(=O)c1ccc(NC(=O)c2ccc(CN(C(=O)c3ccc4c(c3)OCC(=O)N4)C3CC3)cc2)cc1] | 967.91 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](N)CCCNC(N)═N)═O | |
| 54 | 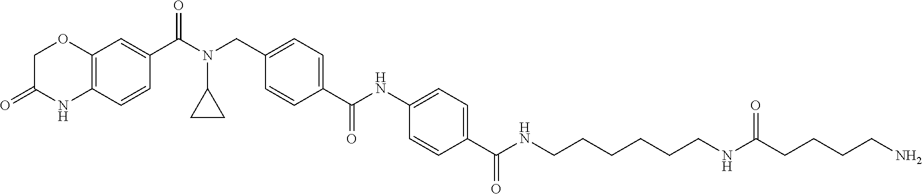 [SMILES: NCCCCC(=O)NCCCCCCNC(=O)c1ccc(NC(=O)c2ccc(CN(C(=O)c3ccc4c(c3)OCC(=O)N4)C3CC3)cc2)cc1] | 796.83 |
| | O═C(C1═CC═C(NC(C2═CC═C(CN(C3CC3)C(C4═CC(OCC(N5)═O)═C5C═C4)═O)C═C2)═O)C═C1)NCCCCCCNC(CCCCN)═O | |
| 55 | 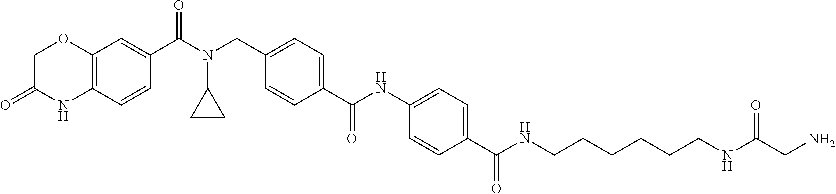 [SMILES: NCC(=O)NCCCCCCNC(=O)c1ccc(NC(=O)c2ccc(CN(C(=O)c3ccc4c(c3)OCC(=O)N4)C3CC3)cc2)cc1] | 754.75 |
| | O═C(C1═CC═C(NC(C2═CC═C(CN(C3CC3)C(C4═CC(OCC(N5)═O)═C5C═C4)═O)C═C2)═O)C═C1)NCCCCCCNC(CN)═O | |
| 56 | 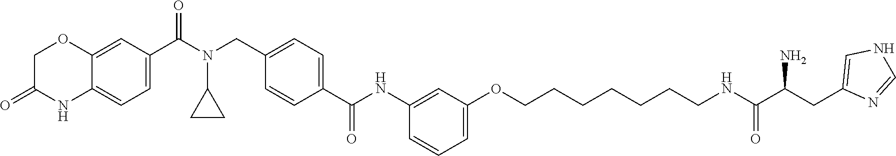 [SMILES: N[C@@H](Cc1c[nH]cn1)C(=O)NCCCCCCCOc1cccc(NC(=O)c2ccc(CN(C(=O)c3ccc4c(c3)OCC(=O)N4)C3CC3)cc2)c1] | 935.86 |
| | O═C(N(C1CC1)CC2═CC═C(C(NC3═CC(OCCCCCCCNC([C@H](CC4═CNC═N4)N)═O)═CC═C3)═O)C═C2)C5═CC═C6C(OCC(N6)═O)═C5 | |
| 57 | 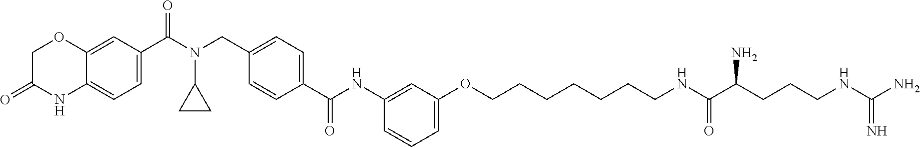 [SMILES: N=C(N)NCCC[C@H](N)C(=O)NCCCCCCCOc1cccc(NC(=O)c2ccc(CN(C(=O)c3ccc4c(c3)OCC(=O)N4)C3CC3)cc2)c1] | 954.91 |
| | O═C(N(C1CC1)CC2═CC═C(C(NC3═CC(OCCCCCCCNC([C@@H](N)CCCNC(N)═N)═O)═CC═C3)═O)C═C2)C4═CC═C5C(OCC(N5)═O)═C4 | |
| 58 | 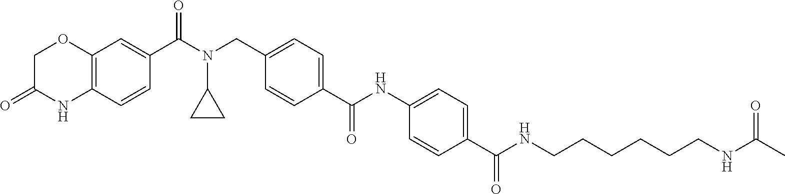 [SMILES: CC(=O)NCCCCCCNC(=O)c1ccc(NC(=O)c2ccc(CN(C(=O)c3ccc4c(c3)OCC(=O)N4)C3CC3)cc2)cc1] | 625.71 |
| | O═C(C1═CC═C(NC(C2═CC═C(CN(C3CC3)C(C4═CC(OCC(N5)═O)═C5C═C4)═O)C═C2)═O)C═C1)NCCCCCCNC(C)═O | |
| 59 | 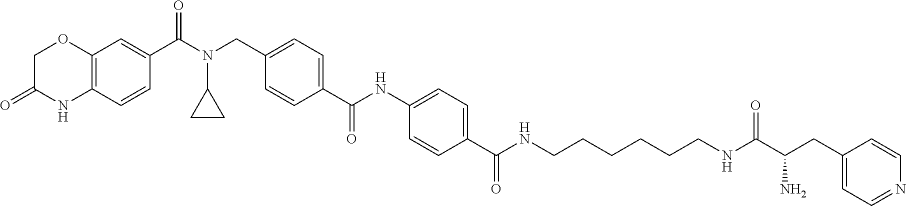 [SMILES: N[C@@H](Cc1ccncc1)C(=O)NCCCCCCNC(=O)c1ccc(NC(=O)c2ccc(CN(C(=O)c3ccc4c(c3)OCC(=O)N4)C3CC3)cc2)cc1] | 959.89 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CC6═CC═NC═C6)═O | |
| 60 | 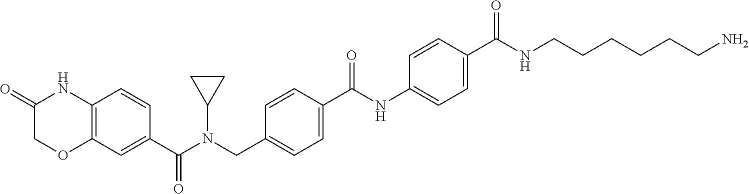 [SMILES: NCCCCCCNC(=O)c1ccc(NC(=O)c2ccc(CN(C(=O)c3ccc4c(c3)OCC(=O)N4)C3CC3)cc2)cc1] | 697.7 |
| | NCCCCCCNC(C1═CC═C(NC(C2═CC═C(CN(C3CC3)C(C4═CC(OCC(N5)═O)═CSC═C4)═O)C═(2)═O)C═C1)═O | |
| 61 | 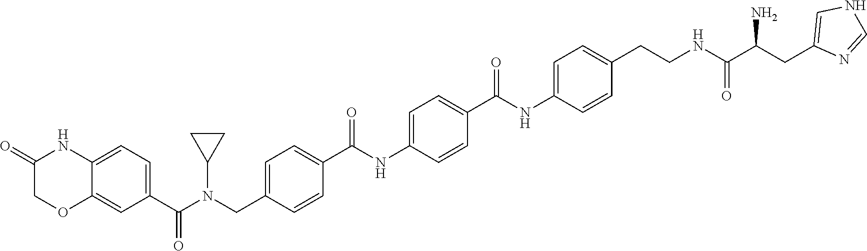 [SMILES: N[C@@H](Cc1c[nH]cn1)C(=O)NCCc1ccc(NC(=O)c2ccc(NC(=O)c3ccc(CN(C(=O)c4ccc5c(c4)OCC(=O)N5)C4CC4)cc3)cc2)cc1] | 968.85 |
| | O═C(N(C1CC1)CC2═CC═C(C(NC3═CC═C(C(NC4═CC═C(CCNC([C@H](CC5═CNC═N5)N)═O)C═C4)═O)C═C3)═O)C═C2)C6═CC═C7C(OCC(N7)═O)═C6 | |
| 62 | 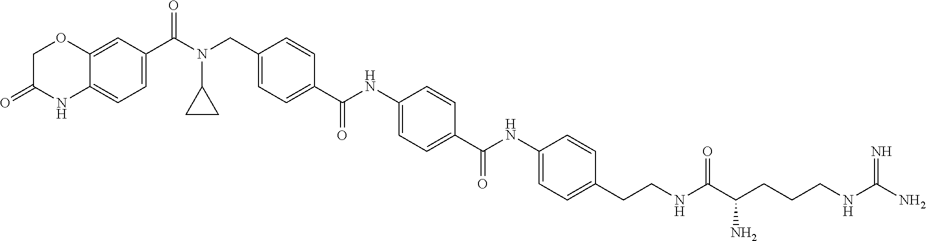 [SMILES: N=C(N)NCCC[C@H](N)C(=O)NCCc1ccc(NC(=O)c2ccc(NC(=O)c3ccc(CN(C(=O)c4ccc5c(c4)OCC(=O)N5)C4CC4)cc3)cc2)cc1] | 987.9 |
| | O═C(N(C1CC1)CC2═C═C(C(NC3═CC═C/C(NC4═CC═C(CNC[C@@H](N)CCCNC(N)═N)═O)C═C4)═O)C═C3)═O)C═C2)C5═CC═C6C(OCC(N6)═O)═C5 | |
| 63 | 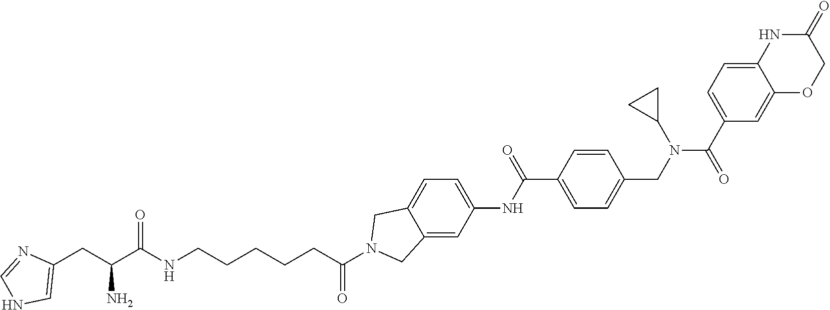 [SMILES: N[C@@H](Cc1c[nH]cn1)C(=O)NCCCCCC(=O)N1Cc2ccc(NC(=O)c3ccc(CN(C(=O)c4ccc5c(c4)OCC(=O)N5)C4CC4)cc3)cc2C1] | 960.87 |
| | O═C(N(C1CC1)CC2═CC═C(C(NC3═CC═C(CN(C(CCCCCNC([C@H](CC4═CNC═N4)N)═O)═O)C5)C5═C3)═O)C═C2)C6═CC═C7C(OCC(N7)═O)═C6 | |
| 64 | 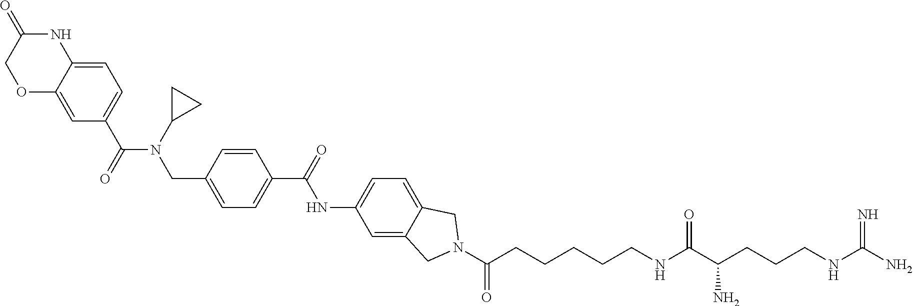 [SMILES: N=C(N)NCCC[C@H](N)C(=O)NCCCCCC(=O)N1Cc2ccc(NC(=O)c3ccc(CN(C(=O)c4ccc5c(c4)OCC(=O)N5)C4CC4)cc3)cc2C1] | 979.92 |
| | O═C(N(C1CC1)CC2═CC═C(C(NC3═CC═C(CN(C(CCCCCNC([C@@H](N)CCCNC(N)═N)═O)═O)C4)C4═C3)═O)C═C2)C5═CC═C6C(OCC(N6)═O)═C5 | |
| 65 | 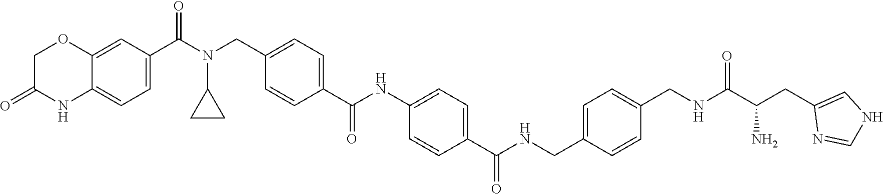 [SMILES: N[C@@H](Cc1c[nH]cn1)C(=O)NCc1ccc(CNC(=O)c2ccc(NC(=O)c3ccc(CN(C(=O)c4ccc5c(c4)OCC(=O)N5)C4CC4)cc3)cc2)cc1] | 968.85 |
| | O═C(N(C1CC1)CC2═CC═C(C(NC3═CC═C(C(NCC4═CC═C(CNC([C@H](CC5═CNC═N5)N)═O)C═C4)═O)C═C3)═O)C═C2)C6═CC═C7C(OCC(N7)═O)═C6 | |
| 66 | 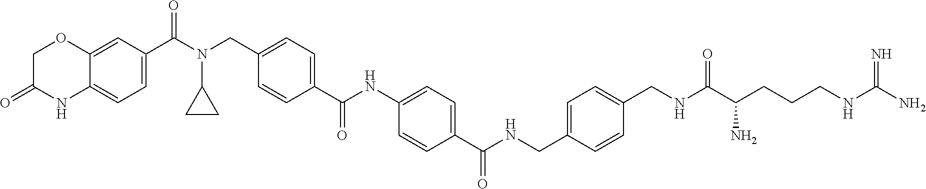 [SMILES: N=C(N)NCCC[C@H](N)C(=O)NCc1ccc(CNC(=O)c2ccc(NC(=O)c3ccc(CN(C(=O)c4ccc5c(c4)OCC(=O)N5)C4CC4)cc3)cc2)cc1] | 987.9 |
| | O═C(N(C1CC1)CC2═CC═C(C(NC3═CC═C(C(NCC4═CC═C(CNC([C@@H](N)CCCNC(N)═N)═O)C═C4)═O)C═C3)═O)C═C2)C5═CC═C6C(OCC(N6)═O)═C5 | |
| 67 | 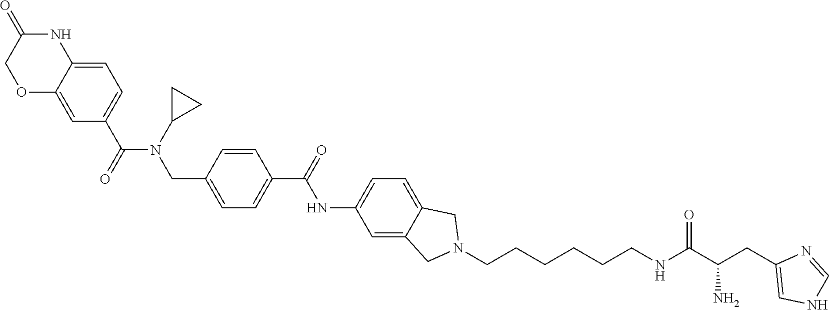 [SMILES: N[C@@H](Cc1c[nH]cn1)C(=O)NCCCCCCN1Cc2ccc(NC(=O)c3ccc(CN(C(=O)c4ccc5c(c4)OCC(=O)N5)C4CC4)cc3)cc2C1] | 1060.9 |
| | O═C(N(C1CC1)CC2═CC═C(C(NC3═CC═C(CN(CCCCCCNC([C@H](CC4═CNC═N4)N)═O)C5)C5═C3)═O)C═C2)C6═CC═C7C(OCC(N7)═O)═C6 | |
| 68 | 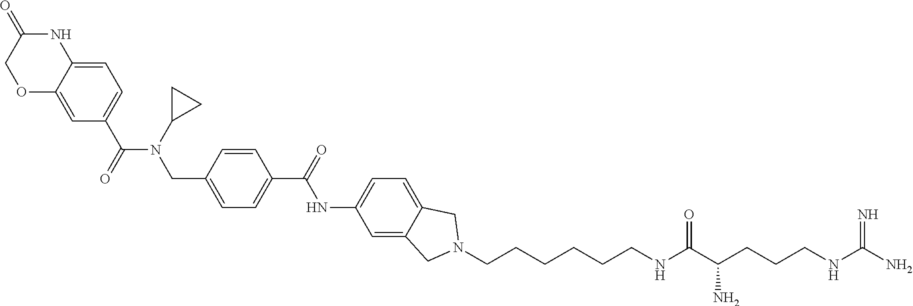 [SMILES: N=C(N)NCCC[C@H](N)C(=O)NCCCCCCN1Cc2ccc(NC(=O)c3ccc(CN(C(=O)c4ccc5c(c4)OCC(=O)N5)C4CC4)cc3)cc2C1] | 1080 |
| | O═C(N(C1CC1)CC2═CC═C(C(NC3═CC═C(CN(CCCCCCNC([C@@H](N)CCCNC(N)═N)═O)C4)C4═C3)═O)C═C2)C5═CC═C6C(OCC(N6)═O)═C5 | |
| 69 | 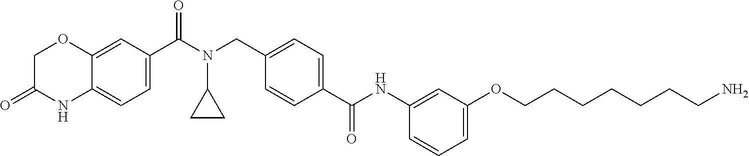 [SMILES: NCCCCCCCOc1cccc(NC(=O)c2ccc(CN(C(=O)c3ccc4c(c3)OCC(=O)N4)C3CC3)cc2)c1] | 911.84 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](CCN)N)═O | |
| 70 | 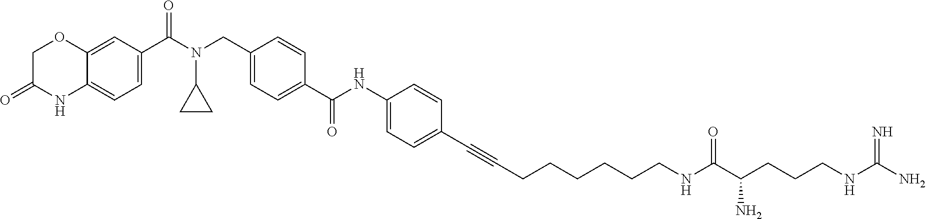 [SMILES: N=C(N)NCCC[C@H](N)C(=O)NCCCCCCC#Cc1ccc(NC(=O)c2ccc(CN(C(=O)c3ccc4c(c3)OCC(=O)N4)C3CC3)cc2)cc1] | 948.91 |
| | O═C(N(C1CC1)CC2═CC═C(C(NC3═CC═C(C#CCCCCCCNC([C@@H](N)CCCNC(N)═N}═O)C═C3)═O)C═C2)C4═CC═C5C(OCC(N5)═O)═C4 | |
| 71 | 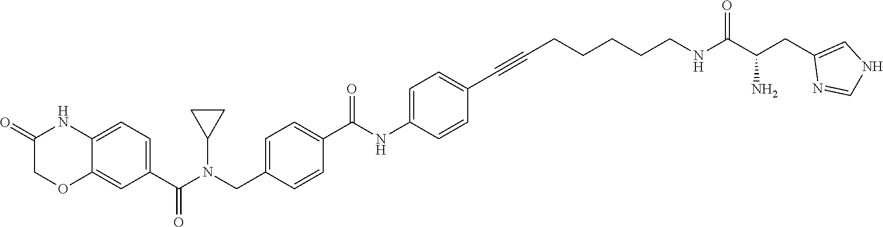 [SMILES: N[C@@H](Cc1c[nH]cn1)C(=O)NCCCCCC#Cc1ccc(NC(=O)c2ccc(CN(C(=O)c3ccc4c(c3)OCC(=O)N4)C3CC3)cc2)cc1] | 915.83 |
| | O═C(N(C1CC1)CC2═CC═C(C(NC3═CC═C(C#CCCCCCNC([C@H](CC4═CNC═N4)N)═O)C═C3)═O)C═C2)C5═CC═C6C(OCC(N6)═O)═C5 | |
| 72 | 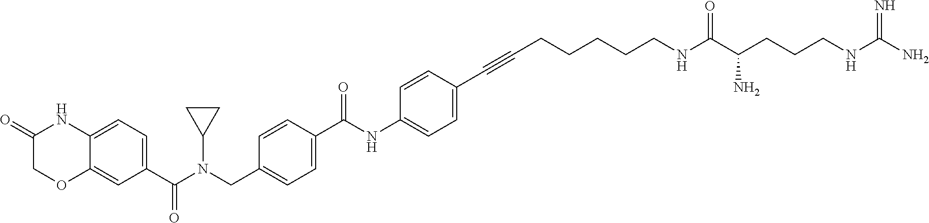 [SMILES: N=C(N)NCCC[C@H](N)C(=O)NCCCCCC#Cc1ccc(NC(=O)c2ccc(CN(C(=O)c3ccc4c(c3)OCC(=O)N4)C3CC3)cc2)cc1] | 934.88 |
| | O═C(N(C1CC1)CC2═CC═C(C(NC3═CC═C(C#CCCCCCNC([C@@H](N)CCCNC(N)═N)═O)C═C3)═O)C═C2)C4═CC═C5C(OCC(N5)═O)═C4 | |
| 73 | 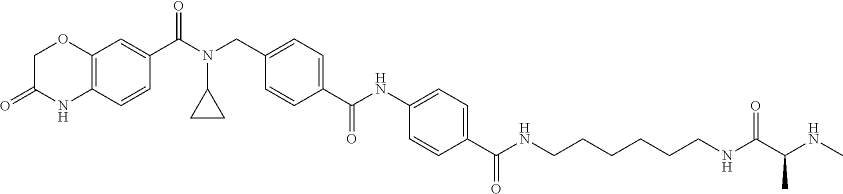 [SMILES: CN[C@@H](C)C(=O)NCCCCCCNC(=O)c1ccc(NC(=O)c2ccc(CN(C(=O)c3ccc4c(c3)OCC(=O)N4)C3CC3)cc2)cc1] | 782.8 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](NC)C)═O | |
| 74 | 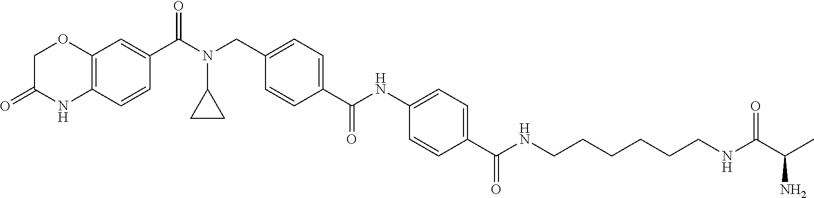 [SMILES: C[C@@H](N)C(=O)NCCCCCCNC(=O)c1ccc(NC(=O)c2ccc(CN(C(=O)c3ccc4c(c3)OCC(=O)N4)C3CC3)cc2)cc1] | 768.78 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](N)C)═O | |
| 75 | 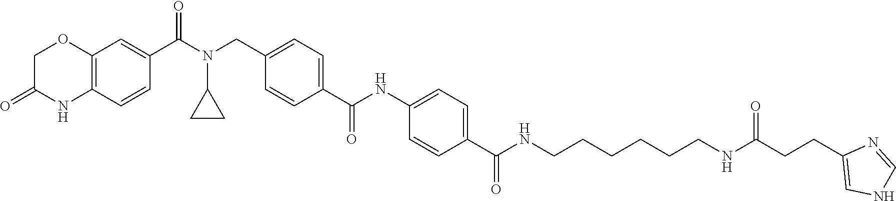 [SMILES: O=C(CCc1c[nH]cn1)NCCCCCCNC(=O)c1ccc(NC(=O)c2ccc(CN(C(=O)c3ccc4c(c3)OCC(=O)N4)C3CC3)cc2)cc1] | 819.83 |
| | O═C(C1═CC═C(NC(C2═CC═C(CN(C3CC3)C(C4═CC(OCC(N5)═O)═C5C═C4)═O)C═C2)═O)C═C1)NCCCCCCNC(CCC6═CNC═N6)═O | |
| 76 | 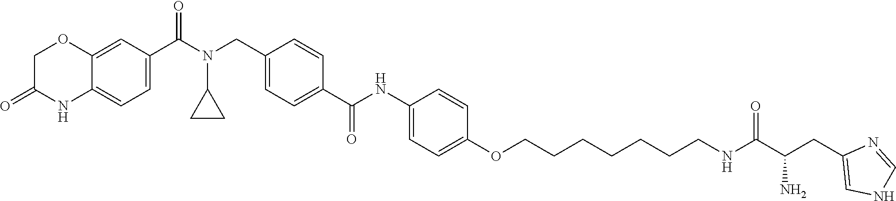 [SMILES: N[C@@H](Cc1c[nH]cn1)C(=O)NCCCCCCCOc1ccc(NC(=O)c2ccc(CN(C(=O)c3ccc4c(c3)OCC(=O)N4)C3CC3)cc2)cc1] | 935.86 |
| | O═C(N(C1CC1)CC2═CC═C(C(NC3═CC═C(OCCCCCCCNC([C@H](CC4═CNC═N4)N)═O)C═C3)═O)C═C2)C5═CC═C6C(OCC(N6)═O)═C5 | |
| 77 | 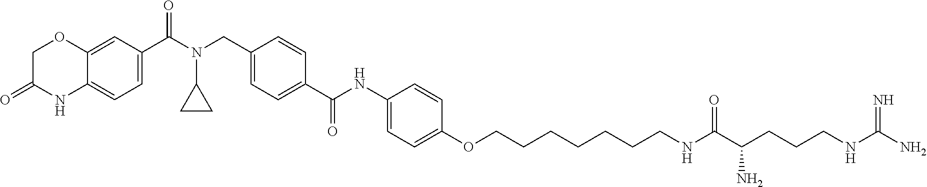 [SMILES: N=C(N)NCCC[C@H](N)C(=O)NCCCCCCCOc1ccc(NC(=O)c2ccc(CN(C(=O)c3ccc4c(c3)OCC(=O)N4)C3CC3)cc2)cc1] | 954.91 |
| | O═C(N(C1CC1)CC2═CC═C(C(NC3═CC═C(OCCCCCCCNC([C@@H](N)CCCNC(N)═N)═O)C═C3)═O)C═C2)C4═CC═C5C(OCC(N5)═O)═C4 | |
| 78 | 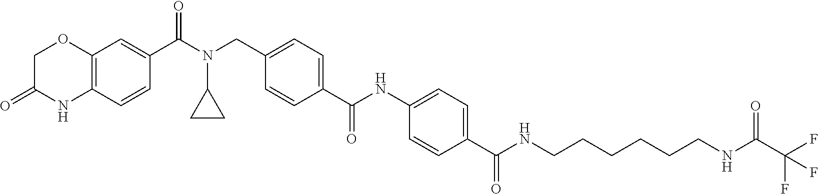 [SMILES: O=C1COc2cc(C(=O)N(Cc3ccc(C(=O)Nc4ccc(C(=O)NCCCCCCNC(=O)C(F)(F)F)cc4)cc3)C3CC3)ccc2N1] | 679.69 |
| | O═C(C1═CC═C(NC(C2═CC═C(CN(C3CC3)C(C4═CC(OCC(N5)═O)═C5C═C4)═O)C═C2)═O)C═C1)NCCCCCCNC(C(F)(F)F)═O | |
| 79 | 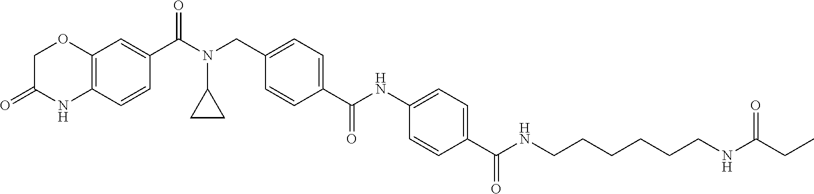 [SMILES: CCC(=O)NCCCCCCNC(=O)c1ccc(NC(=O)c2ccc(CN(C(=O)c3ccc4c(c3)OCC(=O)N4)C3CC3)cc2)cc1] | 639.74 |
| | O═C(C1═CC═C(NC(C2═CC═C(CN(C3CC3)C(C4═CC(OCC(N5)═O)═C5C═C4)═O)C═C2)═O)C═C1)NCCCCCCNC(CC)═O | |
| 80 | 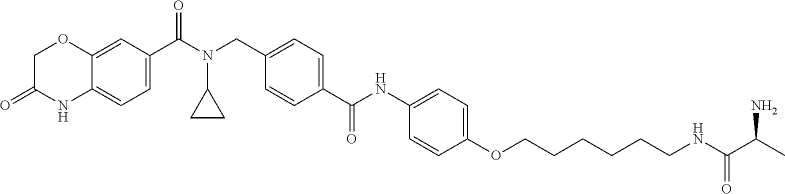 [SMILES: C[C@H](N)C(=O)NCCCCCCOc1ccc(NC(=O)c2ccc(CN(C(=O)c3ccc4c(c3)OCC(=O)N4)C3CC3)cc2)cc1] | 741.75 |
| | O═C(N(C1CC1)CC2═CC═C(C(NC3═CC═C(OCCCCCCNC([C@@H](N)C)═O)C═C3)═O)C═C2)C4═CC═C5C(OCC(N5)═O)═C4 | |
| 81 | 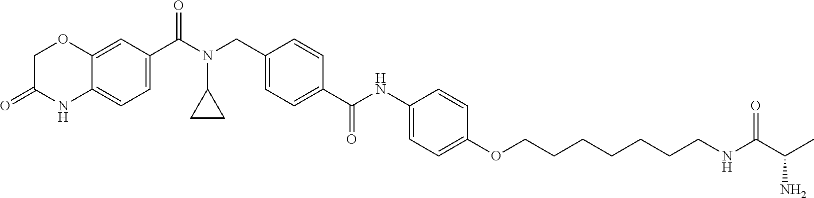 [SMILES: C[C@H](N)C(=O)NCCCCCCCOc1ccc(NC(=O)c2ccc(CN(C(=O)c3ccc4c(c3)OCC(=O)N4)C3CC3)cc2)cc1] | 755.78 |
| | O═C(N(C1CC1)CC2═CC═C(C(NC3═CC═C(OCCCCCCCNC([C@@H](N)C)═O)C═C3)═O)C═C2)C4═CC═C5C(OCC(N5)═O)═C4 | |
| 82 | 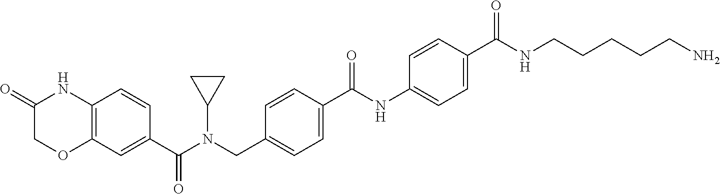 [SMILES: NCCCCCNC(=O)c1ccc(NC(=O)c2ccc(CN(C(=O)c3ccc4c(c3)OCC(=O)N4)C3CC3)cc2)cc1] | 683.67 |
| | NCCCCCNC(C(C═C1)═CC═C1NC(C(C═C2)═CC═C2CN(C(C(C═C3)═CC4═C3NC(CO4)═O)═O)C5CC5)═O)═O | |
| 83 | 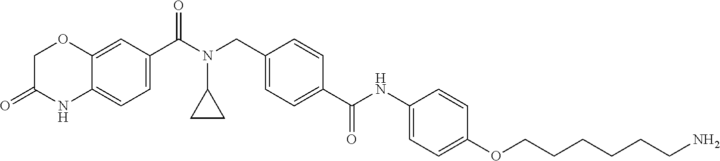 [SMILES: NCCCCCCOc1ccc(NC(=O)c2ccc(CN(C(=O)c3ccc4c(c3)OCC(=O)N4)C3CC3)cc2)cc1] | 670.68 |
| | O═C(N(C1CC1)CC2═CC═C(C(NC3═CC═C(OCCCCCCN)C═C3)═O)C═C2)C4═CC═C5C(OCC(N5)═O)═C4 | |
| 84 | 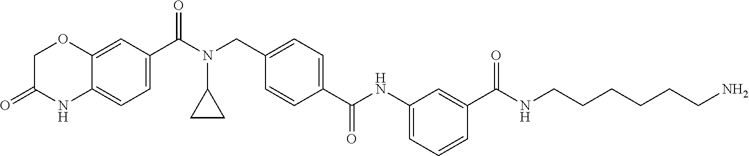 [SMILES: NCCCCCCNC(=O)c1cccc(NC(=O)c2ccc(CN(C(=O)c3ccc4c(c3)OCC(=O)N4)C3CC3)cc2)c1] | 697.7 |
| | O═C(N(C1CC1)CC2═CC═C(C(NC3═CC═CC(C(NCCCCCCN)═O)═C3)═O)C═C2)C4═CC═C5C(OCC(N5)═O)═C4 | |
| 85 | 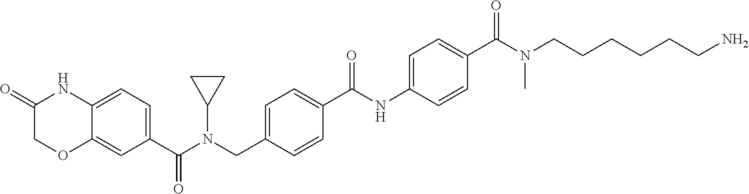 [SMILES: CN(CCCCCCN)C(=O)c1ccc(NC(=O)c2ccc(CN(C(=O)c3ccc4c(c3)OCC(=O)N4)C3CC3)cc2)cc1] | 711.73 |
| | O═C(N(C1CC1)CC2═CC═C(C(NC3═CC═C(C(N(C)CCCCCCN)═O)C═C3)═O)C═C2)C4═CC═C5C(OCC(N5)═O)═C4 | |
| 86 | 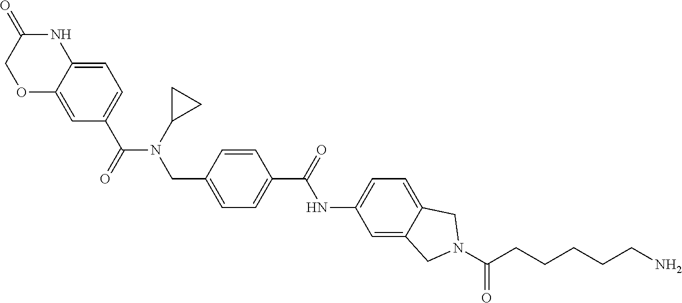 [SMILES: NCCCCCC(=O)N1Cc2ccc(NC(=O)c3ccc(CN(C(=O)c4ccc5c(c4)OCC(=O)N5)C4CC4)cc3)cc2C1] | 709.71 |
| | O═C(N(C1CC1)CC2═CC═C(C(NC3═CC═C(CN(C(CCCCCN)═O)C4)C4═C3)═O)C═C2)C5═CC═C6C(OCC(N6)═O)═C5 | |
| 87 | 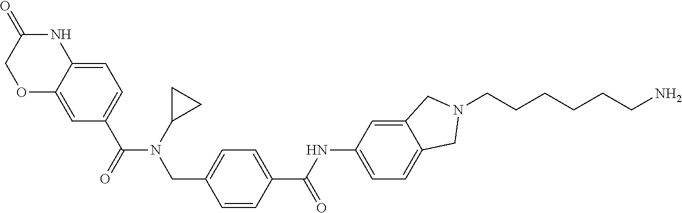 [SMILES: NCCCCCCN1Cc2ccc(NC(=O)c3ccc(CN(C(=O)c4ccc5c(c4)OCC(=O)N5)C4CC4)cc3)cc2C1] | 809.75 |
| | NCCCCCCN(C1)CC(C1═C2)═CC═C2NC(C(C═C3)═CC═C3CN(C4CC4)C(C5═CC═C6C(OCC(N6)═O)═C5)═O)═O | |
| 88 | 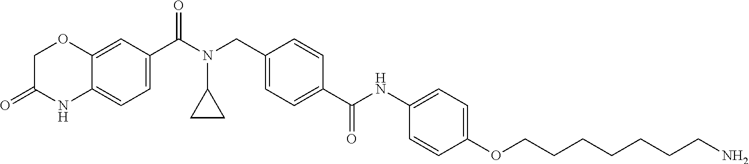 [SMILES: NCCCCCCCOc1ccc(NC(=O)c2ccc(CN(C(=O)c3ccc4c(c3)OCC(=O)N4)C3CC3)cc2)cc1] | 684.7 |
| | NCCCCCCCOC1═CC═C(NC(C(C═C2)═CC═C2CN(C3CC3)C(C4═CC═C5C(OCC(N5)═O)═C4)═O)}═O)C═C1 | |
| 89 | 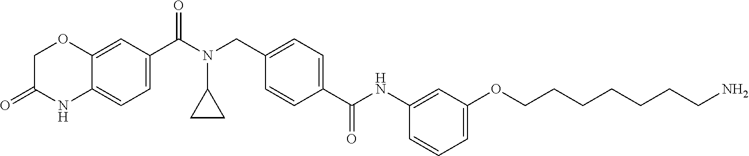 [SMILES: NCCCCCCCOc1cccc(NC(=O)c2ccc(CN(C(=O)c3ccc4c(c3)OCC(=O)N4)C3CC3)cc2)c1] | 684.7 |
| | O═C(N(C1CC1)CC2═CC═C(C(NC3═CC(OCCCCCCCN)═CC═C3)═O)C═C2)C4═CC═C5C(OCC(N5)═O)═ | |
| 90 | 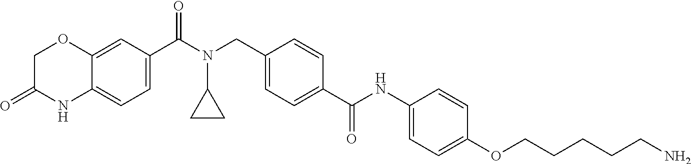 [SMILES: NCCCCCOc1ccc(NC(=O)c2ccc(CN(C(=O)c3ccc4c(c3)OCC(=O)N4)C3CC3)cc2)cc1] | 656.65 |
| | O═C(N(C1CC1)CC2═CC═C(C(NC3═CC═C(OCCCCCN)C═C3)═O)C═C2)C4═CC═C5C(OCC(N5)═0)═C4 | |
| 91 | 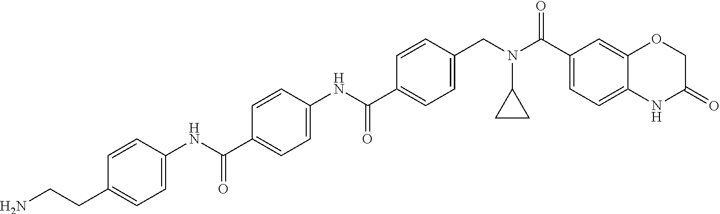 [SMILES: NCCc1ccc(NC(=O)c2ccc(NC(=O)c3ccc(CN(C(=O)c4ccc5c(c4)OCC(=O)N5)C4CC4)cc3)cc2)cc1] | 717.69 |
| | O═C(N(C1CC1)CC2═CC═C(C(NC3═CC═C(C(NC4═CC═C(CCN)C═C4)═O)C═C3)═O)C═C2)C5═CC═C6C(OCC(N6)═0)═C5 | |
| 92 | 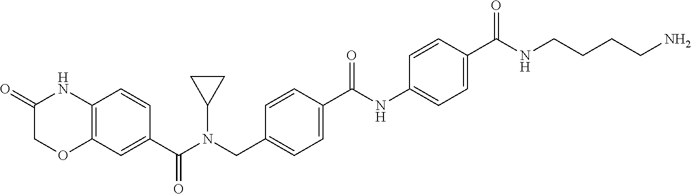 [SMILES: NCCCCNC(=O)c1ccc(NC(=O)c2ccc(CN(C(=O)c3ccc4c(c3)OCC(=O)N4)C3CC3)cc2)cc1] | 669.65 |
| | NCCCCNC(C1═CC═C(NC(C2═CC═C(CN(C3CC3)C(C4═CC(OCC(N5)═0)═C5C═C4)═0)C═C2)═0)C═C1)═0 | |
| 93 | 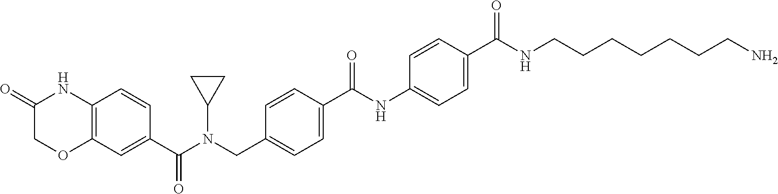 [SMILES: NCCCCCCCNC(=O)c1ccc(NC(=O)c2ccc(CN(C(=O)c3ccc4c(c3)OCC(=O)N4)C3CC3)cc2)cc1] | 711.73 |
| | 0═C(C1═CC═C(NC(C2═CC═C(CN(C3CC3)C(C4═CC(OCC(N5)═0)═C5C═C4)═O)C═C2)═O)C═C1)NCCCCCCCN | |
| 94 | 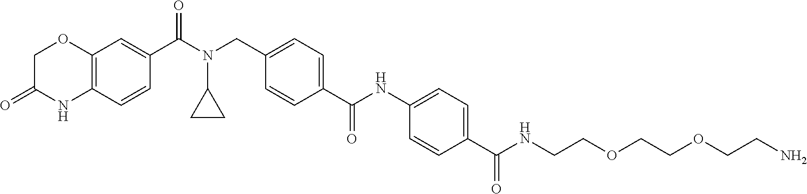 [SMILES: NCCOCCOCCNC(=O)c1ccc(NC(=O)c2ccc(CN(C(=O)c3ccc4c(c3)OCC(=O)N4)C3CC3)cc2)cc1] | 729.7 |
| | O═C(C1═CC═C(NC(C2═CC═C(CN(C3CC3)C(C4═CC(OCC(N5)═O)═C5C═C4)═O)C═C2)═O)C═C1)NCCOCCOCCN | |
| 95 | 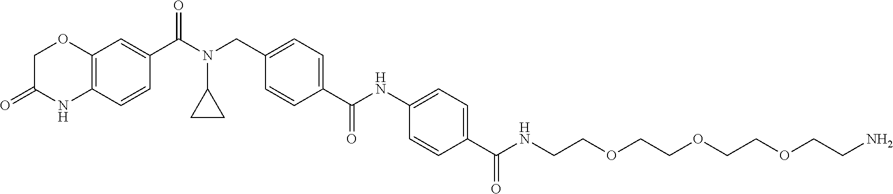 [SMILES: NCCOCCOCCOCCNC(=O)c1ccc(NC(=O)c2ccc(CN(C(=O)c3ccc4c(c3)OCC(=O)N4)C3CC3)cc2)cc1] | 773.75 |
| | O═C(C1═CC═C(NC(C2═CC═C(CN(C3CC3)C(C4═CC(OCC(N5)═0)═C5C═C4)═O)C═C2)═O)C═C1)NCCOCCOCCOCCN | |
| 96 | 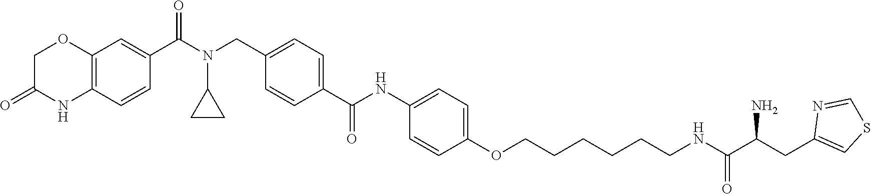 [SMILES: N[C@@H](Cc1cscn1)C(=O)NCCCCCCOc1ccc(NC(=O)c2ccc(CN(C(=O)c3ccc4c(c3)OCC(=O)N4)C3CC3)cc2)cc1] | 824.86 |
| | O═C(C1═CC═C(CN(C2CC2)C(C3═CC(OCC(N4)═O)═C4C═C3)═O)C═C1)NC(C═C5)═CC═C5OCCCCCCNC([C@@H](N)CC6═CSC═N6)═O | |
| 97 | 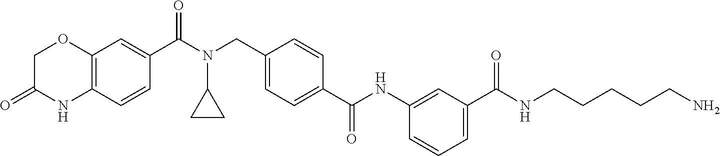 [SMILES: NCCCCCNC(=O)c1cccc(NC(=O)c2ccc(CN(C(=O)c3ccc4c(c3)OCC(=O)N4)C3CC3)cc2)c1] | 683.67 |
| | O═C(C1═CC═C(CN(C2CC2)C(C3═CC(OCC(N4)═O)═C4C═C3)═O)C═C1)NC5═CC(C(NCCCCCN)═O)═CC═C5 | |
| 98 | 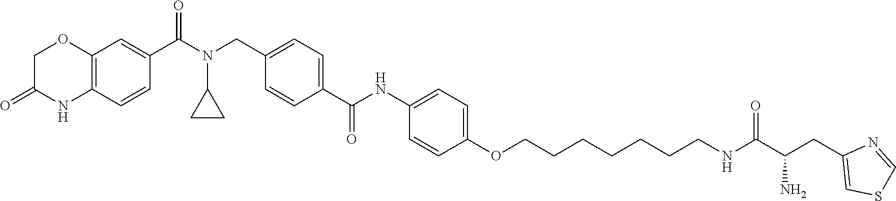 [SMILES: N[C@@H](Cc1cscn1)C(=O)NCCCCCCCOc1ccc(NC(=O)c2ccc(CN(C(=O)c3ccc4c(c3)OCC(=O)N4)C3CC3)cc2)cc1] | 838.89 |
| | O═C(C1═CC═C(CN(C2CC2)C(C3═CC(OCC(N4)═O)═C4C═C3)═O)C═C1)NC(C═C5)═CC═C5OCCCCCCCNC([C@@H](N)CC6═CSC═N6)═O | |
| 99 | 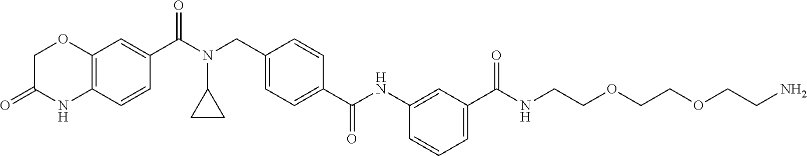 [SMILES: NCCOCCOCCNC(=O)c1cccc(NC(=O)c2ccc(CN(C(=O)c3ccc4c(c3)OCC(=O)N4)C3CC3)cc2)c1] | 729.7 |
| | O═C(C1═CC═C(CN(C2CC2)C(C3═CC(OCC(N4)═O)═C4C═C3)═O)C═C1)NC5═CC(C(NCCOCCOCCN)═O)═CC═C5 | |
| 100 | 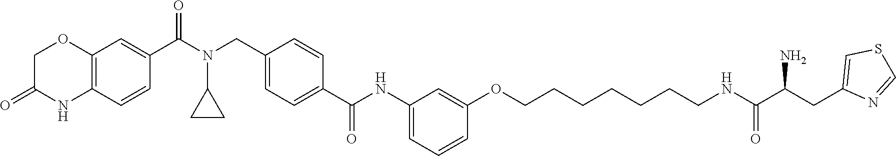 [SMILES: N[C@@H](Cc1cscn1)C(=O)NCCCCCCCOc1cccc(NC(=O)c2ccc(CN(C(=O)c3ccc4c(c3)OCC(=O)N4)C3CC3)cc2)c1] | 838.89 |
| | O═C(C1═CC═C(CN(C2CC2)C(C3═CC(OCC(N4)═O)═C4C═C3)═O)C═C1)NC5═CC═CC(OCCCCCCCNC([C@@H](N)CC6═CSC═N6)═O)═C5 | |
| 101 | 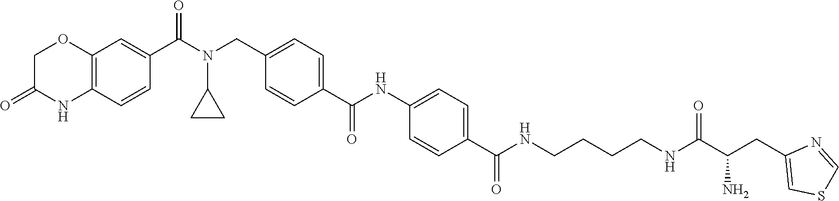 [SMILES: N[C@@H](Cc1cscn1)C(=O)NCCCCNC(=O)c1ccc(NC(=O)c2ccc(CN(C(=O)c3ccc4c(c3)OCC(=O)N4)C3CC3)cc2)cc1] | 823.84 |
| | O═C(C1═CC═C(NC(C2═CC═C(CN(C3CC3)C(C4═CC(OCC(N5)═O)═C5C═C4)═O)C═C2)═O)C═C1)NCCCCNC([C@@H](N)CC6═CSC═N6)═O | |
| 102 | 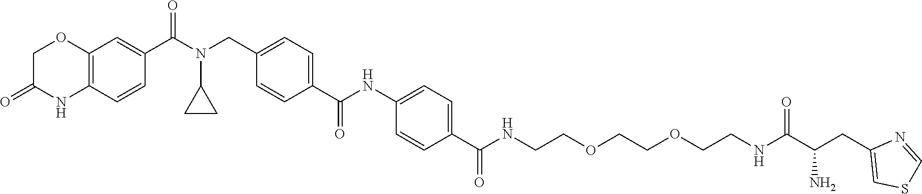 [SMILES: N[C@@H](Cc1cscn1)C(=O)NCCOCCOCCNC(=O)c1ccc(NC(=O)c2ccc(CN(C(=O)c3ccc4c(c3)OCC(=O)N4)C3CC3)cc2)cc1] | 883.89 |
| | O═C(C1═CC═C(NC(C2═CC═C(CN(C3CC3)C(C4═CC(OCC(N5)═O)═C5C═C4)═O)C═C2)═O)C═C1)NCCOCCOCCNC([C@@H](N)CC6═CSC═N6)═O | |
| 103 | 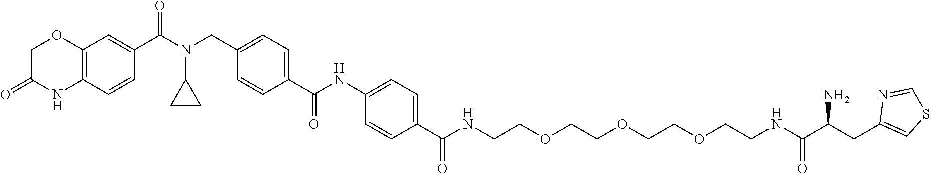 [SMILES: N[C@@H](Cc1cscn1)C(=O)NCCOCCOCCOCCNC(=O)c1ccc(NC(=O)c2ccc(CN(C(=O)c3ccc4c(c3)OCC(=O)N4)C3CC3)cc2)cc1] | 927.94 |
| | O═C(C1═CC═C(NC(C2═CC═C(CN(C3CC3)C(C4═CC(OCC(N5)═O)═C5C═C4)═O)C═C2)═O)C═C1)NCCOCCOCCOCCNC([C@@H](N)CC6═CSC═N6)═O | |
| 104 | 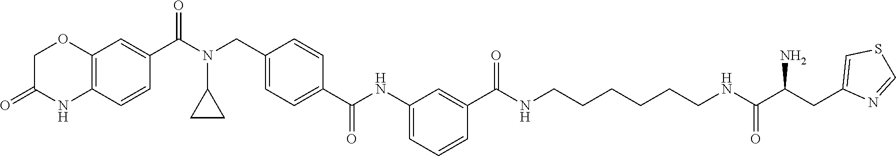 [SMILES: N[C@@H](Cc1cscn1)C(=O)NCCCCCCNC(=O)c1cccc(NC(=O)c2ccc(CN(C(=O)c3ccc4c(c3)OCC(=O)N4)C3CC3)cc2)c1] | 851.89 |
| | O═C(C1═CC═C(CN(C2CC2)C(C3═CC(OCC(N4)═O)═C4C═C3)═O)C═C1)NC5═CC═CC(C(NCCCCCCNC([C@@H](N)CC6═CSC═N6)═O)═O)═C5 | |
| 105 | 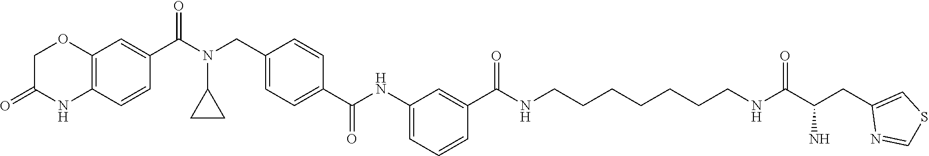 [SMILES: N[C@@H](Cc1cscn1)C(=O)NCCCCCCCNC(=O)c1cccc(NC(=O)c2ccc(CN(C(=O)c3ccc4c(c3)OCC(=O)N4)C3CC3)cc2)c1] | 865.92 |
| | O═C(C1═CC═C(CN(C2CC2)C(C3═CC(OCC(N4)═O)═C4C═C3)═O)C═C1)NC5═CC═CC(C(NCCCCCCCNC([C@@H](N)CC6═CSC═N6)═O)═O)═C5 | |
| 106 | 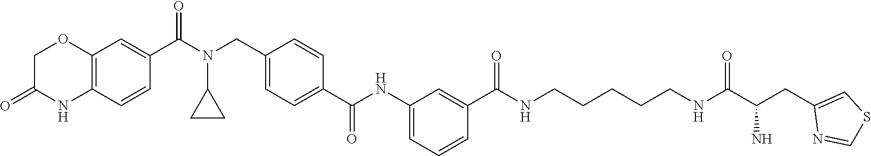 [SMILES: N[C@@H](Cc1cscn1)C(=O)NCCCCCNC(=O)c1cccc(NC(=O)c2ccc(CN(C(=O)c3ccc4c(c3)OCC(=O)N4)C3CC3)cc2)c1] | 837.86 |
| | O═C(C1═CC═C(CN(C2CC2)C(C3═CC(OCC(N4)═O)═C4C═C3)═O)C═C1)NC5═CC═CC(C(NCCCCCNC([C@@H](N)CC6═CSC═N6)═O)═O)═C5 | |
| 107 | 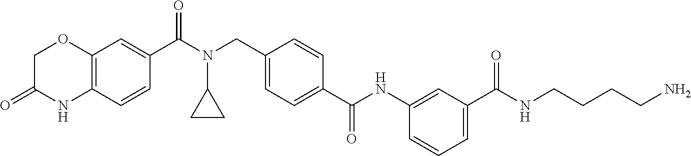 [SMILES: NCCCCNC(=O)c1cccc(NC(=O)c2ccc(CN(C(=O)c3ccc4c(c3)OCC(=O)N4)C3CC3)cc2)c1] | 669.65 |
| | NCCCCNC(C1═CC═CC(NC(C2═CC═C(CN(C3CC3)C(C4═CC(OCC(N5)═O)═C5C═C4)═O)C═C2)═O)═C1)═O | |
| 108 | 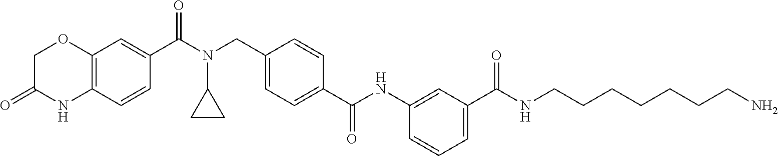 [SMILES: NCCCCCCCNC(=O)c1cccc(NC(=O)c2ccc(CN(C(=O)c3ccc4c(c3)OCC(=O)N4)C3CC3)cc2)c1] | 711.73 |
| | O═C(C1═CC═C(CN(C2CC2)C(C3═CC(OCC(N4)═O)═C4C═C3)═O)C═C1)NC5═CC(C(NCCCCCCCN)═O)═CC═C5 | |
| 109 | 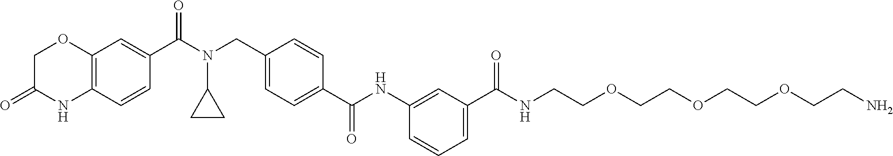 [SMILES: NCCOCCOCCOCCNC(=O)c1cccc(NC(=O)c2ccc(CN(C(=O)c3ccc4c(c3)OCC(=O)N4)C3CC3)cc2)c1] | 773.75 |
| | O═C(C1═CC═C(CN(C2CC2)C(C3═CC(OCC(N4)═O)═C4C═C3)═O)C═C1)NC5═CC(C(NCCOCCOCCOCCN)═O)═CC═C5 | |
| 110 | 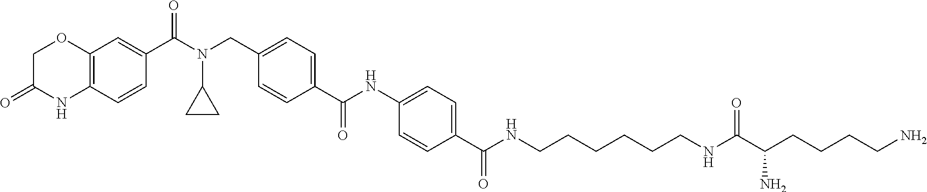 [SMILES: NCCCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 939.9 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CCCCN)═O | |
| 111 | 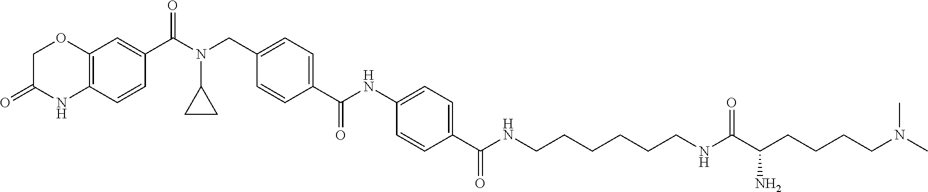 [SMILES: CN(C)CCCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 967.95 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CCCCN(C)C)═O | |
| 112 | 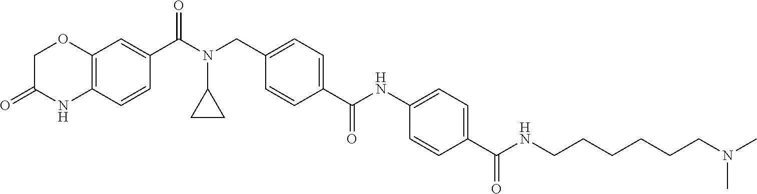 [SMILES: CN(C)CCCCCCNC(=O)c1ccc(NC(=O)c2ccc(CN(C(=O)c3ccc4c(c3)OCC(=O)N4)C3CC3)cc2)cc1] | 725.75 |
| | O═C(C1═CC═C(NC(C2═CC═C(CN(C3CC3)C(C4═CC(OCC(N5)═0)═C5C═C4)═O)C═C2)═O)C═C1)NCCCCCCN(C)C | |
| 113 | 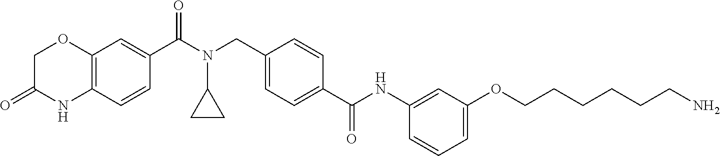 [SMILES: NCCCCCCOc1cccc(NC(=O)c2ccc(CN(C(=O)c3ccc4c(c3)OCC(=O)N4)C3CC3)cc2)c1] | 670.68 |
| | O═C(C1═CC═C(CN(C2CC2)C(C3═CC(OCC(N4)═O)═C4C═C3)═O)C═C1)NC5═CC(OCCCCCCN)═CC═C5 | |
| 114 | 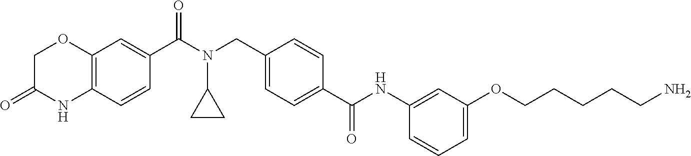 [SMILES: NCCCCCOc1cccc(NC(=O)c2ccc(CN(C(=O)c3ccc4c(c3)OCC(=O)N4)C3CC3)cc2)c1] | 656.65 |
| | O═C(C1═CC═C(CN(C2CC2)C(C3═CC(OCC(N4)═O)═C4C═C3)═O)C═C1)NC5═CC(OCCCCCN)═CC═C5 | |
| 115 | 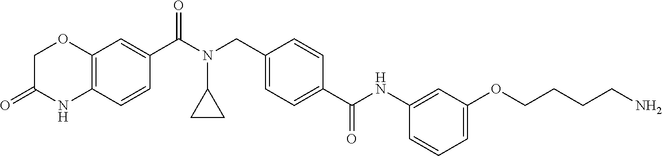 [SMILES: NCCCCOc1cccc(NC(=O)c2ccc(CN(C(=O)c3ccc4c(c3)OCC(=O)N4)C3CC3)cc2)c1] | 642.62 |
| | O═C(C1═CC═C(CN(C2CC2)C(C3═CC(OCC(N4)═O)═C4C═C3)═O)C═C1)NC5═CC(OCCCCN)═CC═C5 | |
| 116 | 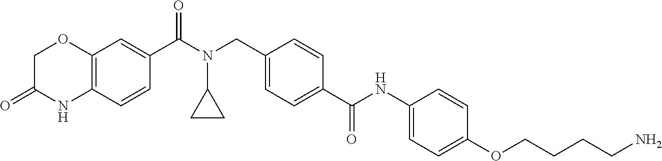 [SMILES: NCCCCOc1ccc(NC(=O)c2ccc(CN(C(=O)c3ccc4c(c3)OCC(=O)N4)C3CC3)cc2)cc1] | 642.62 |
| | O═C(C1═CC═C(CN(C2CC2)C(C3═CC(OCC(N4)═O)═C4C═C3)═O)C═C1)NC5═CC═C(OCCCCN)C═C5 | |
| 117 | 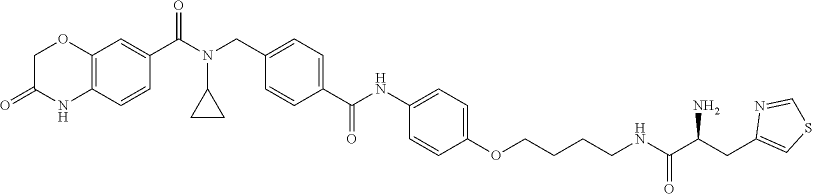 [SMILES: N[C@@H](Cc1cscn1)C(=O)NCCCCOc1ccc(NC(=O)c2ccc(CN(C(=O)c3ccc4c(c3)OCC(=O)N4)C3CC3)cc2)cc1] | 796.81 |
| | O═C(C1═CC═C(CN(C2CC2)C(C3═CC(OCC(N4)═O)═C4C═C3)═O)C═C1)NC(C═C5)═CC═C50CCCCNC([C@@H](N)CC6═CSC═N6)═0 | |
| 118 | 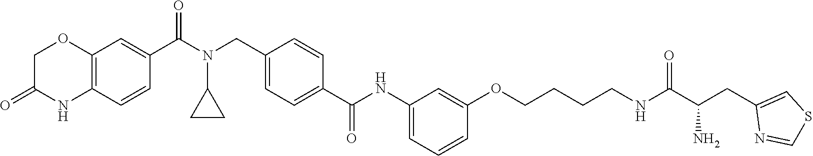 [SMILES: N[C@@H](Cc1cscn1)C(=O)NCCCCOc1cccc(NC(=O)c2ccc(CN(C(=O)c3ccc4c(c3)OCC(=O)N4)C3CC3)cc2)c1] | 796.81 |
| | O═C(C1═CC═C(CN(C2CC2)C(C3═CC(OCC(N4)═O)═C4C═C3)═O)C═C1)NC5═CC═CC(OCCCCNC([C@ | |
| | @H](N)CC6═CSC═N6)═O)═C5 | |
| 119 | 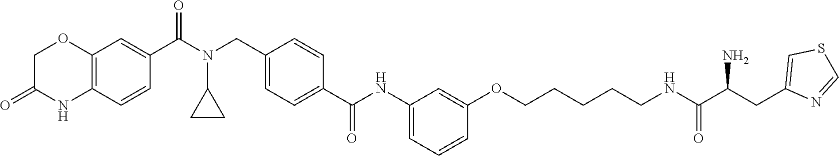 [SMILES: N[C@@H](Cc1cscn1)C(=O)NCCCCCOc1cccc(NC(=O)c2ccc(CN(C(=O)c3ccc4c(c3)OCC(=O)N4)C3CC3)cc2)c1] | 810.84 |
| | O═C(C1═CC═C(CN(C2CC2)(C3═CC(OCC(N4)═O)═C4C═C3)═O)C═C1)NC5═CC═CC(OCCCCCNC([C@@H](N)CC6═CSC═N6)═O)═C5 | |
| 120 | 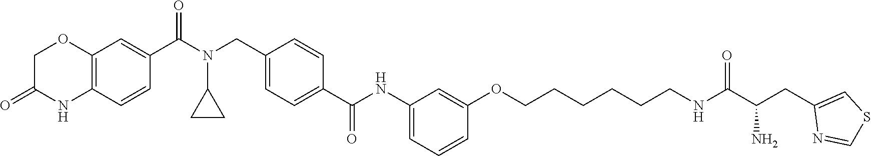 [SMILES: N[C@@H](Cc1cscn1)C(=O)NCCCCCCOc1cccc(NC(=O)c2ccc(CN(C(=O)c3ccc4c(c3)OCC(=O)N4)C3CC3)cc2)c1] | 824.86 |
| | O═C(C1═CC═C(CN(C2CC2)C(C3═CC(OCC(N4)═O)═C4C═C3)═O)C═C1)NC5═CC═CC(OCCCCCCNC([C@@H](N)CC6═CSC═N6)═O)═C5 | |
| 121 | 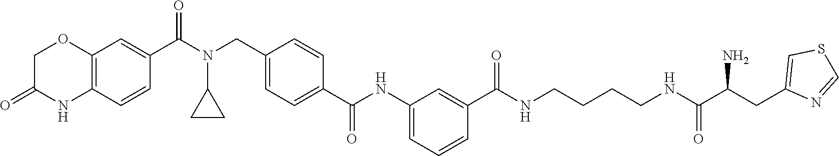 [SMILES: N[C@@H](Cc1cscn1)C(=O)NCCCCNC(=O)c1cccc(NC(=O)c2ccc(CN(C(=O)c3ccc4c(c3)OCC(=O)N4)C3CC3)cc2)c1] | 823.84 |
| | O═C(C1═CC═C(CN(C2CC2)C(C3═CC(OCC(N4)═O)═C4C═C3)═O)C═C1)NC5═CC═CC(C(NCCCCNC([C@@H](N)CC6═CSC═N6)═O)═O)═C5 | |
| 122 | 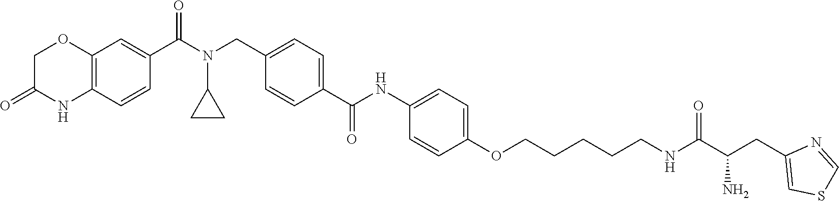 [SMILES: N[C@@H](Cc1cscn1)C(=O)NCCCCCOc1ccc(NC(=O)c2ccc(CN(C(=O)c3ccc4c(c3)OCC(=O)N4)C3CC3)cc2)cc1] | 810.84 |
| | O═C(C1═CC═C(CN(C2CC2)C(C3═CC(OCC(N4)═O)═C4C═C3)═O)C═C1)NC(C═C5)═CC═C5OCCCCCNC([C@@H](N)CC6═CSC═N6)═O | |
| 123 | 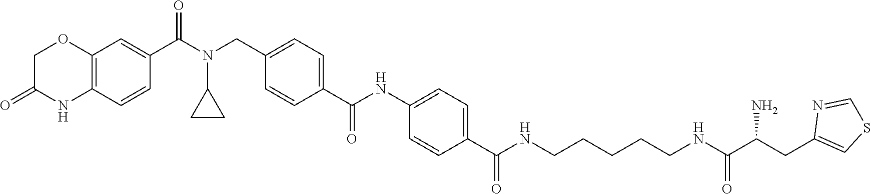 [SMILES: N[C@H](Cc1cscn1)C(=O)NCCCCCNC(=O)c1ccc(NC(=O)c2ccc(CN(C(=O)c3ccc4c(c3)OCC(=O)N4)C3CC3)cc2)cc1] | 837.86 |
| | O═C(C1═CC═C(NC(C2═CC═C(CN(C3CC3)C(C4═CC(OCC(N5)═O)═C5C═C4)═O)C═C2)═O)C═C1)NCCCCCNC([C@H](N)CC6═CSC═N6)═O | |
| 124 | 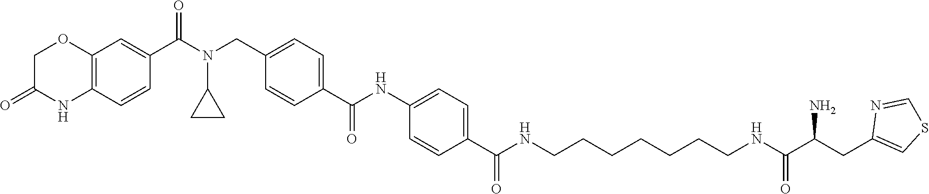 [SMILES: N[C@@H](Cc1cscn1)C(=O)NCCCCCCCNC(=O)c1ccc(NC(=O)c2ccc(CN(C(=O)c3ccc4c(c3)OCC(=O)N4)C3CC3)cc2)cc1] | 865.92 |
| | O═C(C1═CC═C(NC(C2═CC═C(CN(C3CC3)C(C4═CC(OCC(N5)═O)═C5C═C4)═O)C═C2)═O)C═C1)NCCCCCCCNC([C@@H](N)CC6═CSC═N6)═O | |
| 125 | 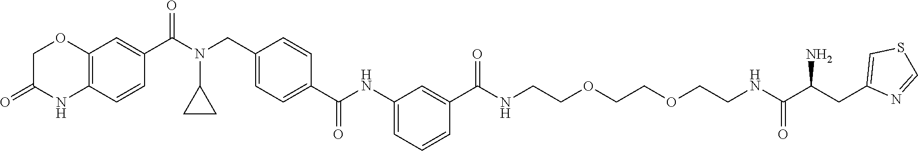 [SMILES: N[C@@H](Cc1cscn1)C(=O)NCCOCCOCCNC(=O)c1cccc(NC(=O)c2ccc(CN(C(=O)c3ccc4c(c3)OCC(=O)N4)C3CC3)cc2)c1] | 883.89 |
| | O═C(C1═CC═C(CN(C2CC2)C(C3═CC(OCC(N4)═O)═C4C═C3)═O)C═C1)NC5═CC═CC(C(NCCOCCOCCNC([C@@H](N)CC6═CSC═N6)═O)═O)═C5 | |
| 126 | 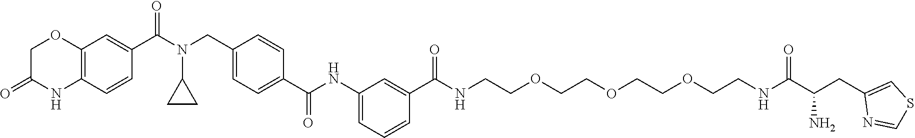 [SMILES: N[C@@H](Cc1cscn1)C(=O)NCCOCCOCCOCCNC(=O)c1cccc(NC(=O)c2ccc(CN(C(=O)c3ccc4c(c3)OCC(=O)N4)C3CC3)cc2)c1] | 927.94 |
| | O═C(C1═CC═C(CN(C2CC2)C(C3═CC(OCC(N4)═O)═C4C═C3)═O)C═C1)NC5═CC═CC(C(NCCOCCOCCOCCNC([C@@H](N)CC6═CSC═N6)═O)═O)═C5 | |
| 127 | 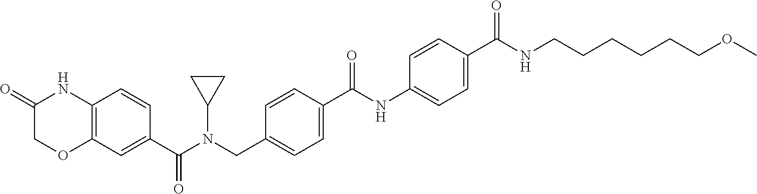 [SMILES: COCCCCCCNC(=O)c1ccc(NC(=O)c2ccc(CN(C(=O)c3ccc4c(c3)OCC(=O)N4)C3CC3)cc2)cc1] | 598.7 |
| | O═C(C1═CC═C(NC(C2═CC═C(CN(C3CC3)C(C4═CC(OCC(N5)═O)═C5C═C4)═O)C═C2)═O)C═C1)NCCCCCCOC | |
| 128 | 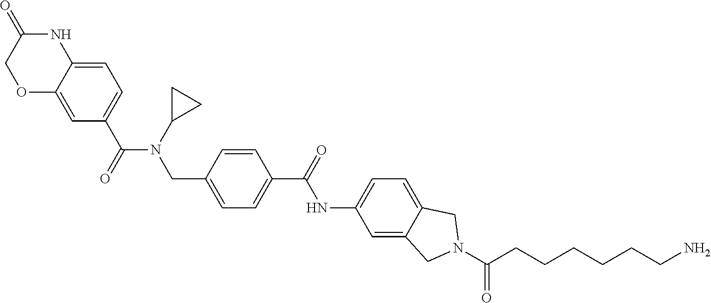 [SMILES: NCCCCCCC(=O)N1Cc2ccc(NC(=O)c3ccc(CN(C(=O)c4ccc5c(c4)OCC(=O)N5)C4CC4)cc3)cc2C1] | 723.74 |
| | O═C(N(C1CC1)CC2═CC═C(C(NC3═CC═C(CN(C(CCCCCCN)═O)C4)C4═C3)═O)C═C2)C5═CC═C6C(OCC(N6)═O)═C5 | |
| 129 | 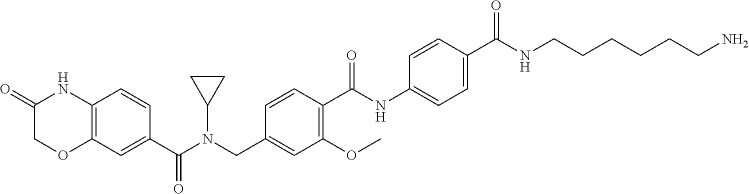 [SMILES: COc1cc(CN(C(=O)c2ccc3c(c2)OCC(=O)N3)C2CC2)ccc1C(=O)Nc1ccc(C(=O)NCCCCCCN)cc1] | 727.73 |
| | NCCCCCCNC(C1═CC═C(NC(C2═C(OC)C═C(CN(C3CC3)C(C4═CC(OCC(N5)═O)═C5C═C4)═O)C═C2)═O)C═C1)═O | |
| 130 | 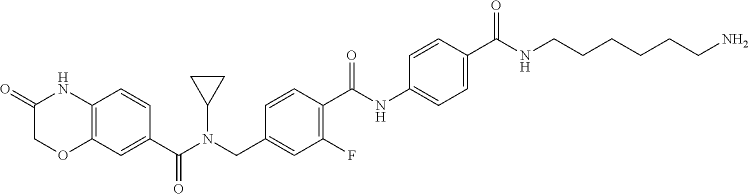 [SMILES: NCCCCCCNC(=O)c1ccc(NC(=O)c2ccc(CN(C(=O)c3ccc4c(c3)OCC(=O)N4)C3CC3)cc2F)cc1] | 715.69 |
| | NCCCCCCNC(C1═CC═C(NC(C2═C(F)C═C(CN(C3CC3)C(C4═CC(OCC(N5)═O)═C5C═C4)═O)C═C2)═O)C═C1)═O | |
| 131 | 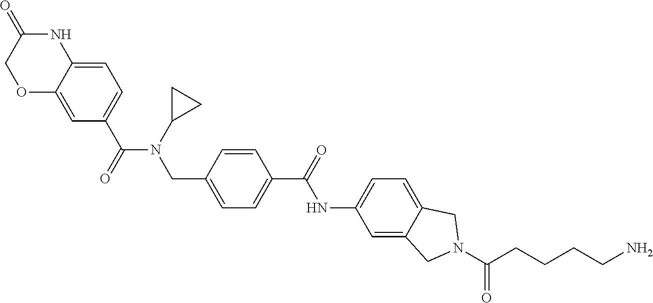 [SMILES: NCCCCC(=O)N1Cc2ccc(NC(=O)c3ccc(CN(C(=O)c4ccc5c(c4)OCC(=O)N5)C4CC4)cc3)cc2C1] | 695.68 |
| | O═C(N(C1CC1)CC2═CC═C(C(NC3═CC═C(CN(C(CCCCN)═O)(4)C4═C3)═O)C═C2)C5═CC═C6C(OCC(N6)═O)═C5 | |
| 132 | 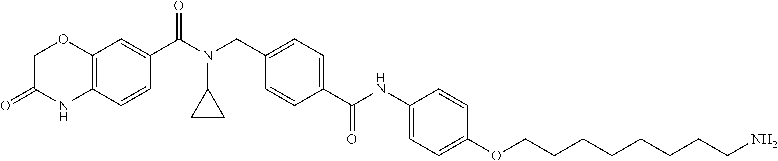 [SMILES: NCCCCCCCCOc1ccc(NC(=O)c2ccc(CN(C(=O)c3ccc4c(c3)OCC(=O)N4)C3CC3)cc2)cc1] | 698.73 |
| | O═C(C1═CC═C(CN(C2CC2)C(C3═CC(OCC(N4)═O)═C4C═C3)═O)C═C1)NC5═CC═C(OCCCCCCCCN)C═C5 | |
| 133 | 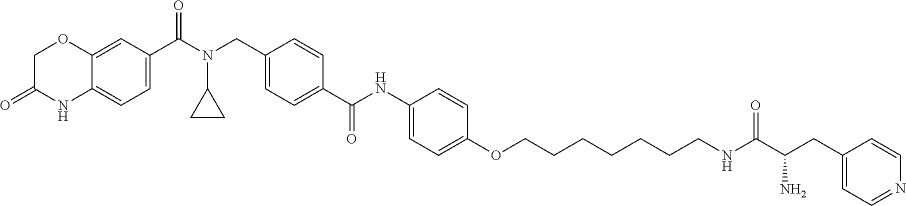 [SMILES: N[C@@H](Cc1ccncc1)C(=O)NCCCCCCCOc1ccc(NC(=O)c2ccc(CN(C(=O)c3ccc4c(c3)OCC(=O)N4)C3CC3)cc2)cc1] | 946.89 |
| | O═C(N(C1CC1)CC2═CC═C(C(NC3═CC═C(OCCCCCCCNC([C@@H](N)CC4═CC═NC═C4)═O)C═C3)═O)C═C2)C5═CC═C6C(OCC(N6)═O)═C5 | |
| 134 | 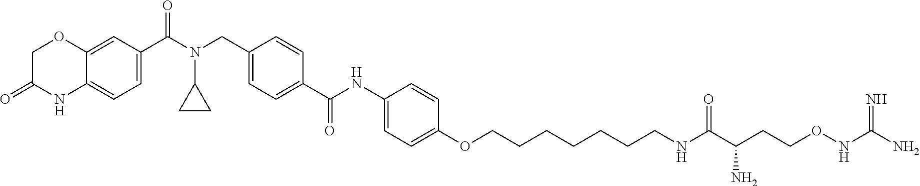 [SMILES: N=C(N)NOCC[C@H](N)C(=O)NCCCCCCCOc1ccc(NC(=O)c2ccc(CN(C(=O)c3ccc4c(c3)OCC(=O)N4)C3CC3)cc2)cc1] | 956.88 |
| | O═C(N(C1CC1)CC2═CC═C(C(NC3═CC═C(OCCCCCCCNC([C@@H](N)CCONC(N)═N)═O)C═C3)═O)C═C2)C4═CC═C5C(OCC(N5)═O)═C4 | |
| 135 | 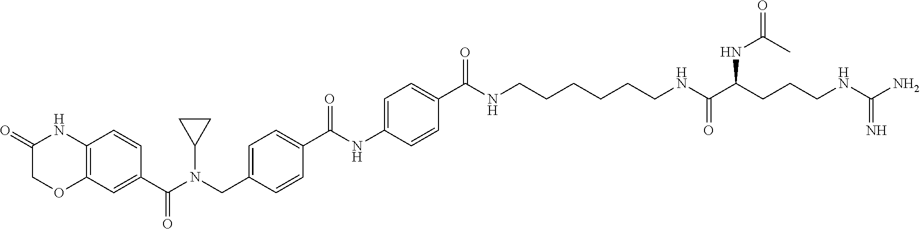 [SMILES: CC(=O)N[C@@H](CCCNC(=N)N)C(=O)NCCCCCCNC(=O)c1ccc(NC(=O)c2ccc(CN(C(=O)c3ccc4c(c3)OCC(=O)N4)C3CC3)cc2)cc1] | 895.92 |
| | CC(N[C@H](C(NCCCCCCNC(C(C═C1)═CC═C1NC(C(C═C2)═CC═C2CN(C(C(C═C3)═CC4═C3NC(CO4)═O)═O)C5CC5)═O)═O)═O)CCCNC(N)═N)═O | |
| 136 | 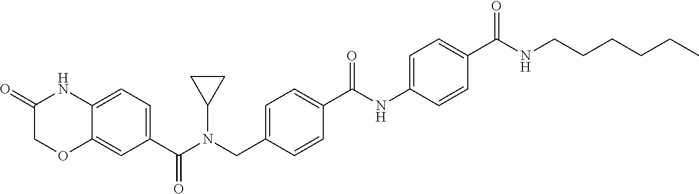 [SMILES: CCCCCCNC(=O)c1ccc(NC(=O)c2ccc(CN(C(=O)c3ccc4c(c3)OCC(=O)N4)C3CC3)cc2)cc1] | 568.66 |
| | O═C(N(C1CC1)CC2═CC═C(C(NC3═CC═C(C(NCCCCCC)═O)C═C3)═O)C═C2)C4═CC═C5C(OCC(N5)═O)═C4 | |
| 137 | 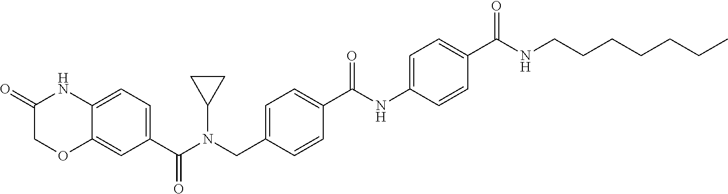 [SMILES: CCCCCCCNC(=O)c1ccc(NC(=O)c2ccc(CN(C(=O)c3ccc4c(c3)OCC(=O)N4)C3CC3)cc2)cc1] | 582.69 |
| | O═C(N(C1CC1)CC2═CC═C(C(NC3═CC═C(C(NCCCCCCC)═O)C═C3)═O)C═C2)C4═CC═C5C(OCC(N5)═O)═C4 | |
| 138 | 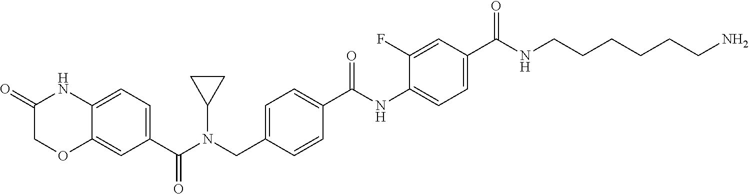 [SMILES: NCCCCCCNC(=O)c1ccc(NC(=O)c2ccc(CN(C(=O)c3ccc4c(c3)OCC(=O)N4)C3CC3)cc2)c(F)c1] | 715.69 |
| | O═C(N(C1CC1)CC2═CC═C(C(NC3═CC═C(C(NCCCCCCN)═O)C═C3F)═O)C═C2)C4═CC═C5C(OCC(N5)═O)═C4 | |
| 139 | 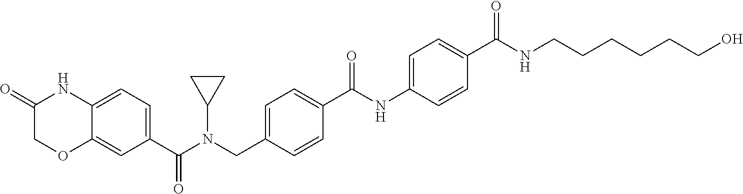 [SMILES: O=C1COc2cc(C(=O)N(Cc3ccc(C(=O)Nc4ccc(C(=O)NCCCCCCO)cc4)cc3)C3CC3)ccc2N1] | 584.66 |
| | O═C(N(C1CC1)CC2═CC═C(C(NC3═CC═C(C(NCCCCCCO)═O)C═C3)═O)C═C2)C4═CC═C5C(OCC(N5)═O)═C4 | |
| 140 | 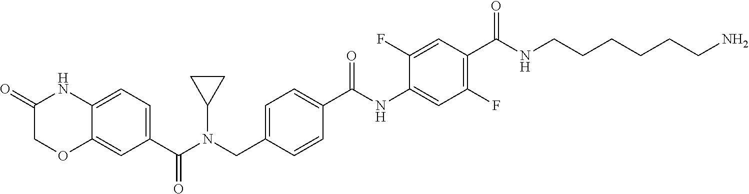 [SMILES: NCCCCCCNC(=O)c1cc(F)c(NC(=O)c2ccc(CN(C(=O)c3ccc4c(c3)OCC(=O)N4)C3CC3)cc2)cc1F] | 733.68 |
| | O═C(N(C1CC1)CC2═CC═C(C(NC3═CC(F)═C(C(NCCCCCCN)═O)C═C3F)═O)C═C2)C4═CC═C5C(OCC(N5)═O)═C4 | |
| 141 | 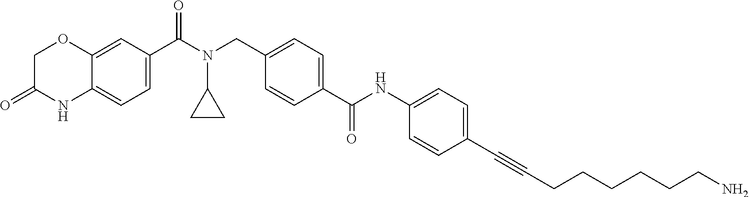 [SMILES: NCCCCCCC#Cc1ccc(NC(=O)c2ccc(CN(C(=O)c3ccc4c(c3)OCC(=O)N4)C3CC3)cc2)cc1] | 678.7 |
| | NCCCCCCC#CC1═CC═C(NC(C2═CC═C(CN(C(C3═CC═C4NC(COC4═C3)═O)═O)C5CC5)C═C2)═O)C═C1 | |
| 142 | 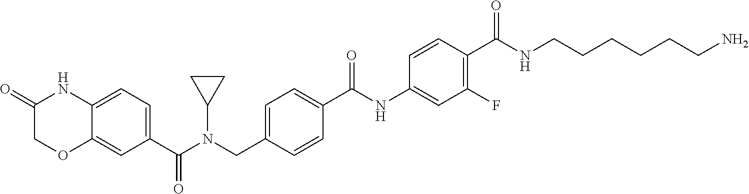 [SMILES: NCCCCCCNC(=O)c1ccc(NC(=O)c2ccc(CN(C(=O)c3ccc4c(c3)OCC(=O)N4)C3CC3)cc2)cc1F] | 715.69 |
| | O═C(N(C1CC1)CC2═CC═C(C(NC3═CC(F)═C(C(NCCCCCCN)═O)C═C3)═O)C═C2)C4═CC═C5C(OCC(N5)═O)═C4 | |
| 143 | 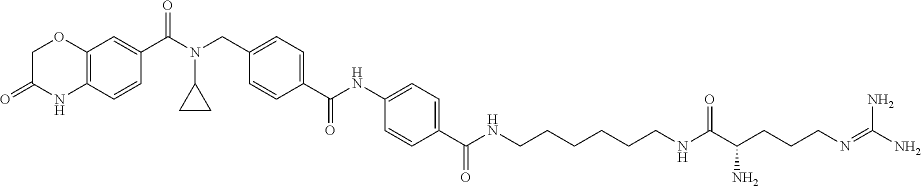 [SMILES: NC(N)=NCCC[C@H](N)C(=O)NCCCCCCNC(=O)c1ccc(NC(=O)c2ccc(CN(C(=O)c3ccc4c(c3)OCC(=O)N4)C3CC3)cc2)cc1] | 967.91 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@H](CCC/N═C(N)/N)N)═O | |
| 144 | 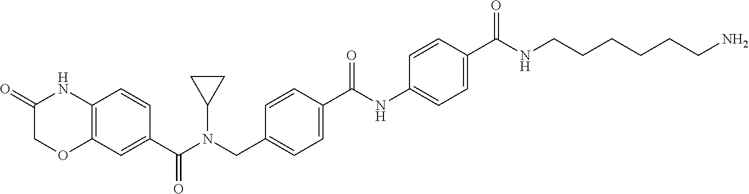 [SMILES: NCCCCCCNC(=O)c1ccc(NC(=O)c2ccc(CN(C(=O)c3ccc4c(c3)OCC(=O)N4)C3CC3)cc2)cc1] | 697.7 |
| | NCCCCCCNC(C1═CC═C(NC(C2═CC═C(CN(C3CC3)C(C4═CC(OCC(N5)═O)═CSC═C4)═O)C═(2)═O)C═C1)═O | |
| 145 | 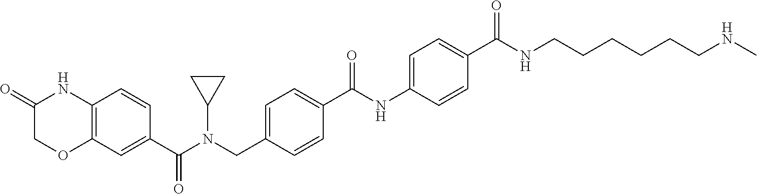 [SMILES: CNCCCCCCNC(=O)c1ccc(NC(=O)c2ccc(CN(C(=O)c3ccc4c(c3)OCC(=O)N4)C3CC3)cc2)cc1] | 711.73 |
| | O═C1NC(C═CC(C(N(CC2═CC═C(C(NC3═CC═C(C(NCCCCCCNC)═O)C═C3)═O)C═C2)C4CC4)═O)═C5)═C5OC1 | |
| 146 | 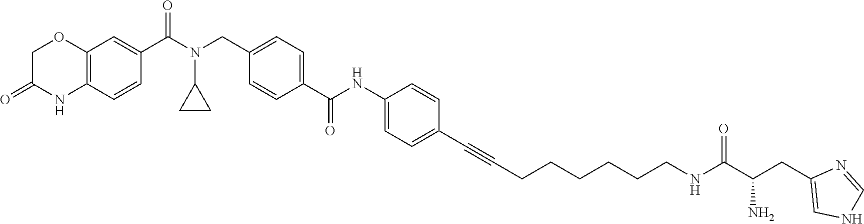 [SMILES: N[C@@H](Cc1c[nH]cn1)C(=O)NCCCCCCC#Cc1ccc(NC(=O)c2ccc(CN(C(=O)c3ccc4c(c3)OCC(=O)N4)C3CC3)cc2)cc1] | 929.86 |
| | O═C1COC(C═C2C(N(C3CC3)CC(C═C4)═CC═C4C(NC5═CC═C(C#CCCCCCCNC([C@@H](N)CC6═CNC═N6)═O)C═C5)═O)═O)═C(C═C2)N1 | |
| 147 | 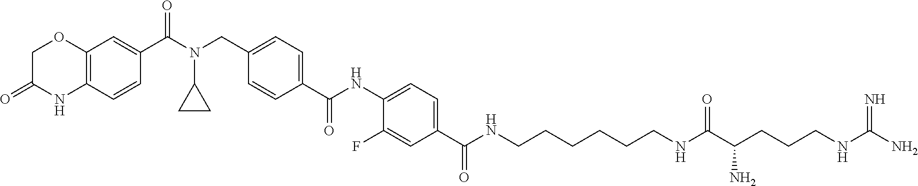 [SMILES: N=C(N)NCCC[C@H](N)C(=O)NCCCCCCNC(=O)c1ccc(NC(=O)c2ccc(CN(C(=O)c3ccc4c(c3)OCC(=O)N4)C3CC3)cc2)c(F)c1] | 985.9 |
| | O═C1COC(C═C2C(N(C3CC3)CC(C═C4)═CC═CAC(NC5═CC═C(C(NCCCCCCNC([C@ @H](N)CCCNC(N)═N)═O)═O)C═C5F)═O)═O)═C(C═C2)N1 | |
| 148 | 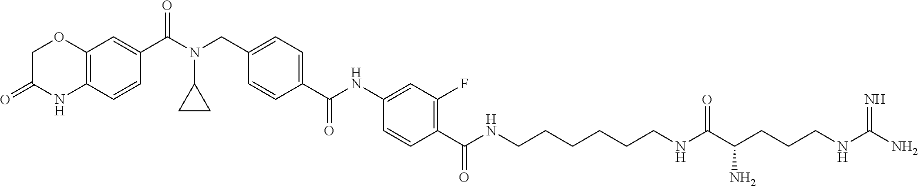 [SMILES: N=C(N)NCCC[C@H](N)C(=O)NCCCCCCNC(=O)c1ccc(NC(=O)c2ccc(CN(C(=O)c3ccc4c(c3)OCC(=O)N4)C3CC3)cc2)cc1F] | 985.9 |
| | O═C1COC(C═C2C(N(C3CC3)CC(C═C4)═CC═C4C(NC5═CC(F)═C(C(NCCCCCCNC([C@@H](N)CCCNC(N)═N)═O)═O)C═C5)═O)═O)═C(C═C2)N1 | |
| 149 | 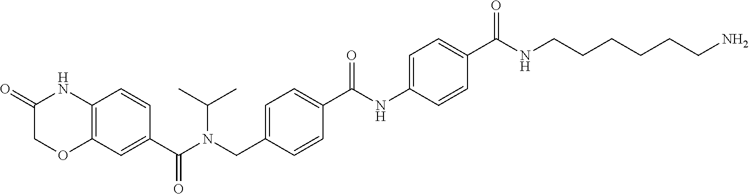 [SMILES: CC(C)N(Cc1ccc(C(=O)Nc2ccc(C(=O)NCCCCCCN)cc2)cc1)C(=O)c1ccc2c(c1)OCC(=O)N2] | 699.72 |
| | CC(N(CC1═CC═C(C(NC2═CC═C(C(NCCCCCCN)═O)C═C2)═O)C═C1)C(C(C═C3)═CC4═C3NC(CO4)═O)═O)C | |
| 150 | 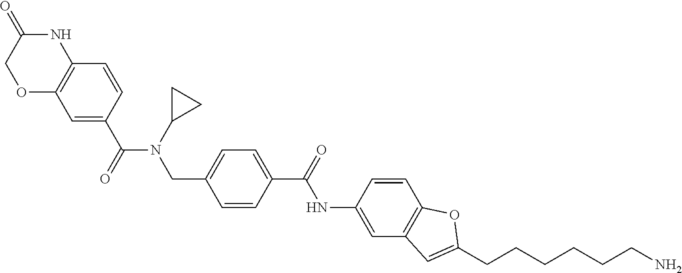 [SMILES: NCCCCCCc1cc2cc(NC(=O)c3ccc(CN(C(=O)c4ccc5c(c4)OCC(=O)N5)C4CC4)cc3)ccc2o1] | 694.7 |
| | O═C1COC(C═C2C(N(C3CC3)CC(C═C4)═CC═C4C(NC5═CC6═C(OC(CCCCCCN)═C6)C═C5)═O)═O)═C(C═C2)N1 | |
| 151 | 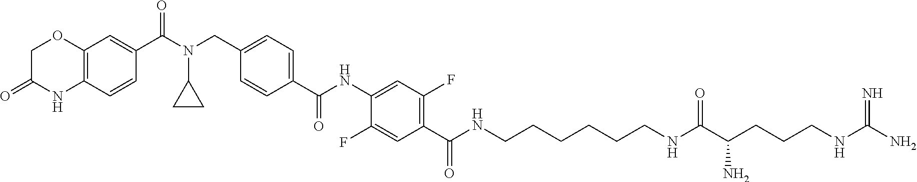 [SMILES: N=C(N)NCCC[C@H](N)C(=O)NCCCCCCNC(=O)c1cc(F)c(NC(=O)c2ccc(CN(C(=O)c3ccc4c(c3)OCC(=O)N4)C3CC3)cc2)cc1F] | 1003.9 |
| | O═C1COC(C═C2C(N(C3CC3)CC(C═C4)═CC═C4C(NC5═CC(F)═C(C(NCCCCCCNC([C@@H](N)CCCNC(N)═N)═O)═O)C═C5F)═O)═O)═C(C═C2)N1 | |
| 152 | 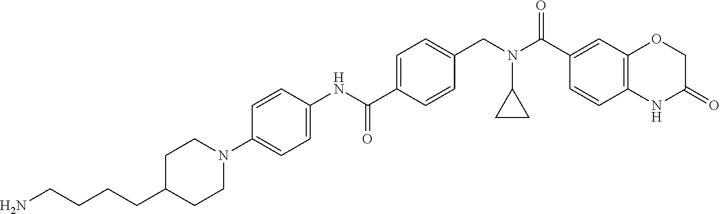 [SMILES: NCCCCC1CCN(c2ccc(NC(=O)c3ccc(CN(C(=O)c4ccc5c(c4)OCC(=O)N5)C4CC4)cc3)cc2)CC1] | 709.75 |
| | O═C1COC(C═C2C(N(C3CC3)CC(C═C4)═CC═C4C(NC5═CC═C(N6CCC(CCCCN)CC6)C═C5)═O)═O)═C(C═C2)N1 | |
| 153 | 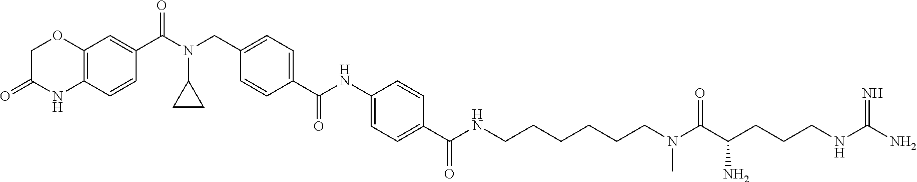 [SMILES: CN(CCCCCCNC(=O)c1ccc(NC(=O)c2ccc(CN(C(=O)c3ccc4c(c3)OCC(=O)N4)C3CC3)cc2)cc1)C(=O)[C@@H](N)CCCNC(=N)N] | 981.94 |
| | O═C1COC(C═C2C(N(C3CC3)CC(C═C4)═CC═CAC(NC5═CC═C(C(NCCCCCCN(C)C([C@@H](N)CCCNC(N)═N)═O)═O)C═C5)═O)═O)═C(C═C2)N1 | |
| 154 | 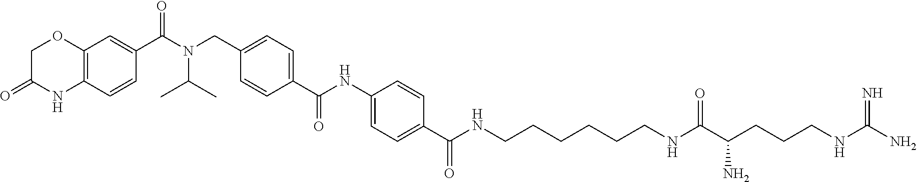 [SMILES: CC(C)N(Cc1ccc(C(=O)Nc2ccc(C(=O)NCCCCCCNC(=O)[C@@H](N)CCCNC(=N)N)cc2)cc1)C(=O)c1ccc2c(c1)OCC(=O)N2] | 969.92 |
| | CC(C)N(C(C1═CC(OC2)═C(C═C1)NC2═O)═O)CC(C═C3)═CC═C3C(NC4═CC═C(C(NCCCCCCNC([C@@H](N)CCCNC(N)═N)═O)═O)C═C4)═O | |
| 155 | 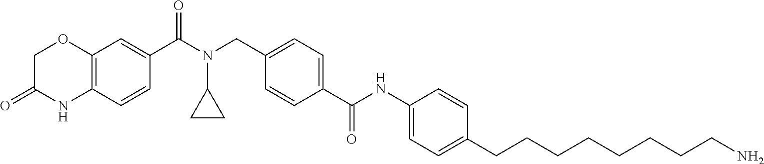 [SMILES: NCCCCCCCCc1ccc(NC(=O)c2ccc(CN(C(=O)c3ccc4c(c3)OCC(=O)N4)C3CC3)cc2)cc1] | 682.73 |
| | O═C(C1═CC═C(CN(C2CC2)C(C3═CC(OCC(N4)═O)═C4C═C3)═O)C═C1)NC5═CC═C(CCCCCCCCN)C═C5 | |
| 156 | 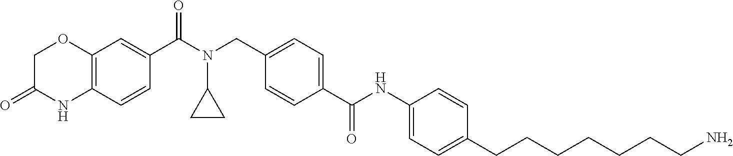 [SMILES: NCCCCCCCc1ccc(NC(=O)c2ccc(CN(C(=O)c3ccc4c(c3)OCC(=O)N4)C3CC3)cc2)cc1] | 668.7 |
| | O═C(C1═CC═C(CN(C2CC2)C(C3═CC(OCC(N4)═O)═C4C═C3)═O)C═C1)NC5═CC═C(CCCCCCCN)C═C5 | |
| 157 | 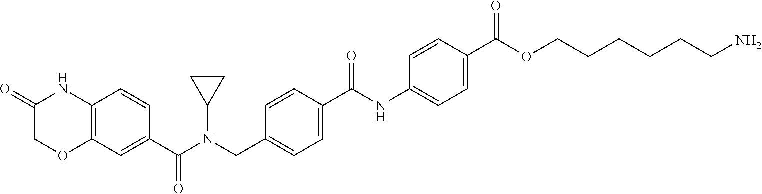 [SMILES: NCCCCCCOC(=O)c1ccc(NC(=O)c2ccc(CN(C(=O)c3ccc4c(c3)OCC(=O)N4)C3CC3)cc2)cc1] | 698.69 |
| | NCCCCCCOC(C1═CC═C(NC(C2═CC═C(CN(C3CC3)C(C4═CC(OCC(N5)═O)═C5C═C4)═O)C═C2)═O)C═C1)═O | |
| 158 | 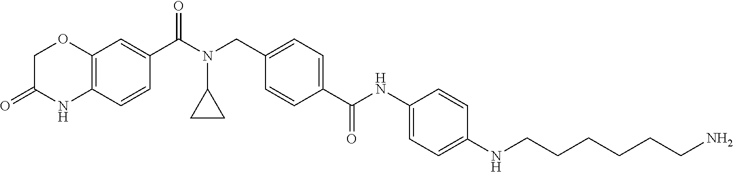 [SMILES: NCCCCCCNc1ccc(NC(=O)c2ccc(CN(C(=O)c3ccc4c(c3)OCC(=O)N4)C3CC3)cc2)cc1] | 669.69 |
| | O═C(C1═CC═C(CN(C2CC2)C(C3═CC(OCC(N4)═O)═C4C═C3)═O)C═C1)NC5═CC═C(NCCCCCCN)C═C5 | |
| 159 | 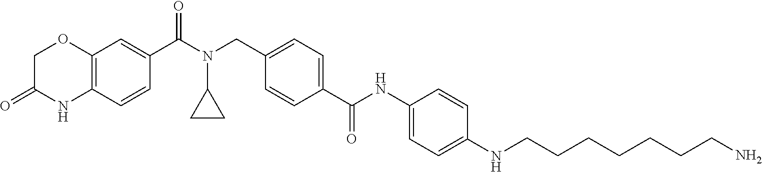 [SMILES: NCCCCCCCNc1ccc(NC(=O)c2ccc(CN(C(=O)c3ccc4c(c3)OCC(=O)N4)C3CC3)cc2)cc1] | 683.72 |
| | O═C(C1═CC═C(CN(C2CC2)C(C3═CC(OCC(N4)═O)═C4C═C3)═O)C═C1)NC5═CC═C(NCCCCCCCN)C═C5 | |
| 160 | 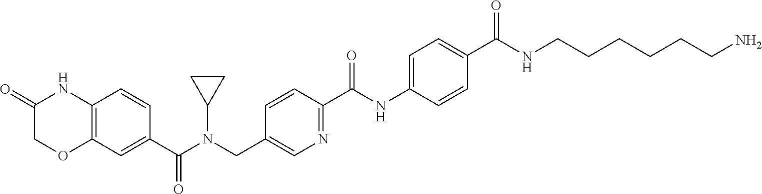 [SMILES: NCCCCCCNC(=O)c1ccc(NC(=O)c2ccc(CN(C(=O)c3ccc4c(c3)OCC(=O)N4)C3CC3)cn2)cc1] | 812.72 |
| | NCCCCCCNC(C1═CC═C(NC(C2═NC═C(CN(C3CC3)C(C4═CC(OCC(N5)═O)═C5C═C4)═O)C═C2)═O)C═C1)═O | |
| 161 | 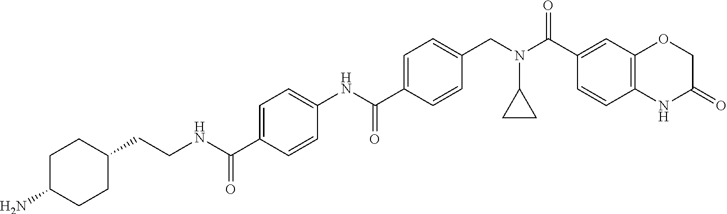 [SMILES: N[C@H]1CC[C@@H](CCNC(=O)c2ccc(NC(=O)c3ccc(CN(C(=O)c4ccc5c(c4)OCC(=O)N5)C4CC4)cc3)cc2)CC1] | 723.74 |
| | O═C(C1═CC═C(NC(C2═CC═C(CN(C3CC3)C(C4═CC(OCC(N5)═O)═C5C═C4)═O)C═C2)═O)C═C1)NCC[C@@H]6CC[C@H](N)CC6 | |
| 162 | 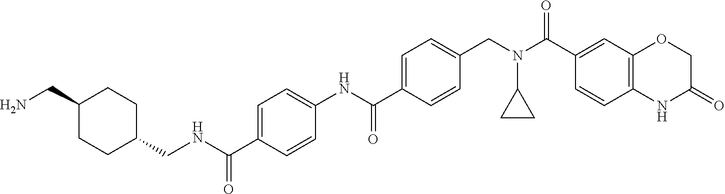 [SMILES: NC[C@H]1CC[C@H](CNC(=O)c2ccc(NC(=O)c3ccc(CN(C(=O)c4ccc5c(c4)OCC(=O)N5)C4CC4)cc3)cc2)CC1] | 723.74 |
| | O═C(C1═CC═C(NC(C2═CC═C(CN(C3CC3)C(C4═CC(OCC(N5)═O)═C5C═C4)═O)C═C2)═O)C═C1)NC[C@@H]6CC[C@@H](CN)CC6 | |
| 163 | 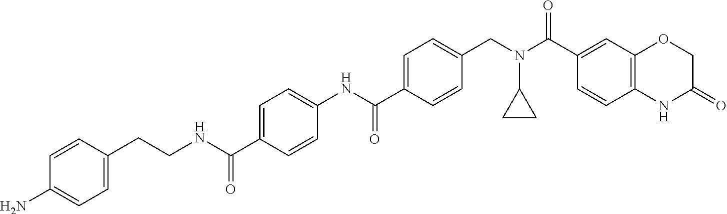 [SMILES: Nc1ccc(CCNC(=O)c2ccc(NC(=O)c3ccc(CN(C(=O)c4ccc5c(c4)OCC(=O)N5)C4CC4)cc3)cc2)cc1] | 603.67 |
| | O═C(C1═CC═C(NC(C2═CC═C(CN(C3CC3)C(C4═CC(OCC(N5)═O)═C5C═C4)═O)C═C2)═O)C═C1)NCCC6═CC═C(N)C═C6 | |
| 164 | 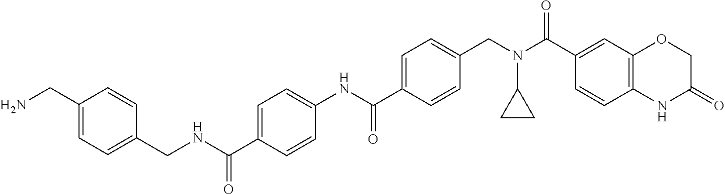 [SMILES: NCc1ccc(CNC(=O)c2ccc(NC(=O)c3ccc(CN(C(=O)c4ccc5c(c4)OCC(=O)N5)C4CC4)cc3)cc2)cc1] | 717.69 |
| | O═C(C1═CC═C(NC(C2═CC═C(CN(C3CC3)C(C4═CC(OCC(N5)═O)═C5C═C4)═O)C═C2)═O)C═C1)NCC6═CC═C(CN)C═C6 | |
| 165 | 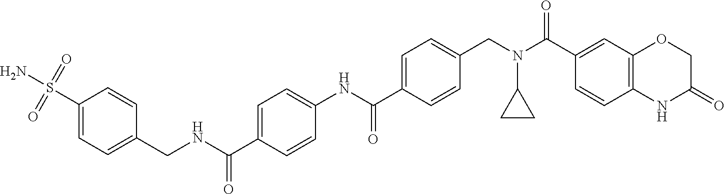 [SMILES: NS(=O)(=O)c1ccc(CNC(=O)c2ccc(NC(=O)c3ccc(CN(C(=O)c4ccc5c(c4)OCC(=O)N5)C4CC4)cc3)cc2)cc1] | 653.7 |
| | O═C(C1═CC═C(NC(C2═CC═C(CN(C3CC3)C(C4═CC(OCC(N5)═O)═C5C═C4)═O)C═C2)═O)C═C1)NCC6═CC═C(S(N)(═O)═O)C═C6 | |
| 166 | 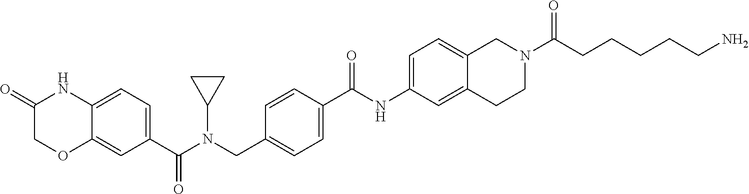 [SMILES: NCCCCCC(=O)N1CCc2cc(NC(=O)c3ccc(CN(C(=O)c4ccc5c(c4)OCC(=O)N5)C4CC4)cc3)ccc2C1] | 723.74 |
| | NCCCCCC(N1CCC2═CC(NC(C3═CC═C(CN(C(C4═CC═C5NC(COC5═C4)═O)═O)C6CC6)C═C3)═O)═CC═C2C1)═O | |
| 167 | 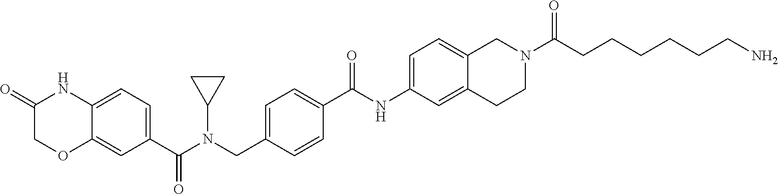 [SMILES: NCCCCCCC(=O)N1CCc2cc(NC(=O)c3ccc(CN(C(=O)c4ccc5c(c4)OCC(=O)N5)C4CC4)cc3)ccc2C1] | 737.76 |
| | O═C(N(C1CC1)CC2═CC═C(C(NC3═CC═C(CN4C(CCCCCCN)═O)C(CC4)═C3)═O)C═C2)C5═CC═C6C(OCC(N6)═O)═C5 | |
| 168 | 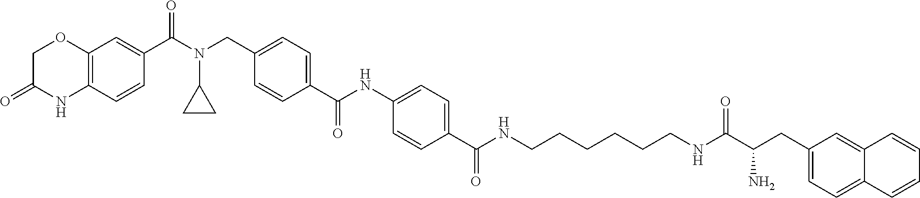 [SMILES: N[C@@H](Cc1ccc2ccccc2c1)C(=O)NCCCCCCNC(=O)c1ccc(NC(=O)c2ccc(CN(C(=O)c3ccc4c(c3)OCC(=O)N4)C3CC3)cc2)cc1] | 894.93 |
| | O═C(C1═CC═C(NC(C2═CC═C(CN(C3CC3)C(C4═CC(OCC(N5)═O)═C5C═C4)═O)C═C2)═O)C═C1)NCCCCCCNC([C@@H](N)CC6═CC═C(C═CC═C7)C7═C6)═O | |
| 169 | 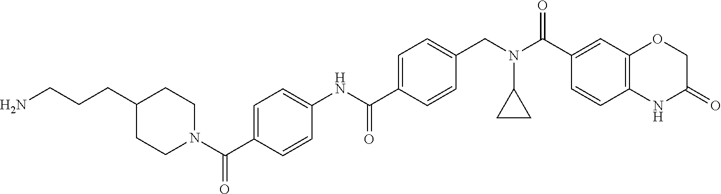 [SMILES: NCCCC1CCN(C(=O)c2ccc(NC(=O)c3ccc(CN(C(=O)c4ccc5c(c4)OCC(=O)N5)C4CC4)cc3)cc2)CC1] | 723.74 |
| | O═C(C1═CC═C(NC(C2═CC═C(CN(C3CC3)C(C4═CC(OCC(N5)═O)═C5C═C4)═O)C═C2)═O)C═C1)N6CCC(CCCN)CC6 | |
| 170 | 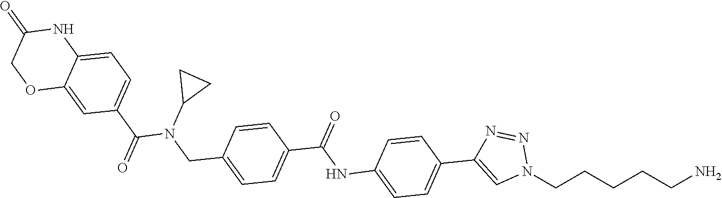 [SMILES: NCCCCCn1cc(-c2ccc(NC(=O)c3ccc(CN(C(=O)c4ccc5c(c4)OCC(=O)N5)C4CC4)cc3)cc2)nn1] | 707.7 |
| | O═C(N(C1CC1)CC2═CC═C(C(NC3═CC═C(C4═CN(CCCCCN)N═N4)C═C3)═O)C═C2)C5═CC═C6C(OCC(N6)═O)═C5 | |
| 171 | 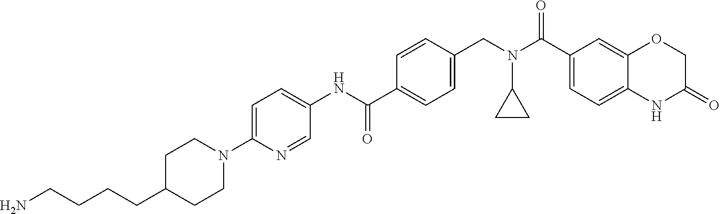 [SMILES: NCCCCC1CCN(c2ccc(NC(=O)c3ccc(CN(C(=O)c4ccc5c(c4)OCC(=O)N5)C4CC4)cc3)cn2)CC1] | 824.77 |
| | NCCCCC1CCN(C2═CC═C(NC(C(C═C3)═CC═C3CN(C4CC4)C(C5═CC═C6C(OCC(N6)═O)═C5)═O)═O)C═N2)CC1 | |
| 172 | 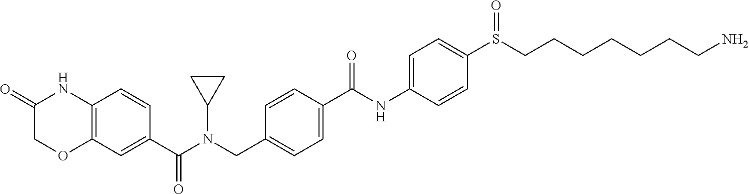 [SMILES: NCCCCCCCS(=O)c1ccc(NC(=O)c2ccc(CN(C(=O)c3ccc4c(c3)OCC(=O)N4)C3CC3)cc2)cc1] | 716.77 |
| | O═C(C1═CC═C(CN(C2CC2)C(C3═CC(OCC(N4)═O)═C4C═C3)═O)C═C1)NC5═CC═C(S(CCCCCCCN)═O)C═C5 | |
| 173 | 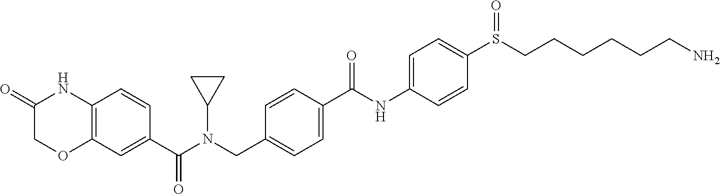 [SMILES: NCCCCCCS(=O)c1ccc(NC(=O)c2ccc(CN(C(=O)c3ccc4c(c3)OCC(=O)N4)C3CC3)cc2)cc1] | 702.74 |
| | O═C(C1═CC═C(CN(C2CC2)C(C3═CC(OCC(N4)═O)═C4C═C3)═O)C═C1)NC5═CC═C(S(CCCCCCN)═O)C═C5 | |
| 174 | 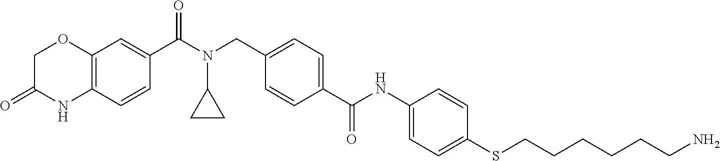 [SMILES: NCCCCCCSc1ccc(NC(=O)c2ccc(CN(C(=O)c3ccc4c(c3)OCC(=O)N4)C3CC3)cc2)cc1] | 686.74 |
| | O═C(C1═CC═C(CN(C2CC2)C(C3═CC(OCC(N4)═O)═C4C═C3)═O)C═C1)NC5═CC═C(SCCCCCCN)C═C5 | |
| 175 | 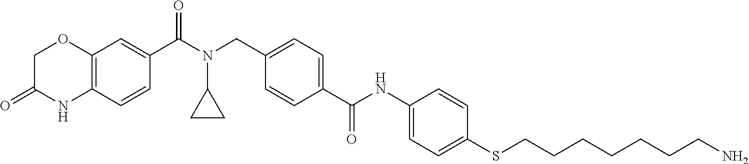 [SMILES: NCCCCCCCSc1ccc(NC(=O)c2ccc(CN(C(=O)c3ccc4c(c3)OCC(=O)N4)C3CC3)cc2)cc1] | 700.77 |
| | O═C(C1═CC═C(CN(C2CC2)C(C3═CC(OCC(N4)═O)═C4C═C3)═O)C═C1)NC5═CC═C(SCCCCCCCN)C═C5 | |
| 176 | 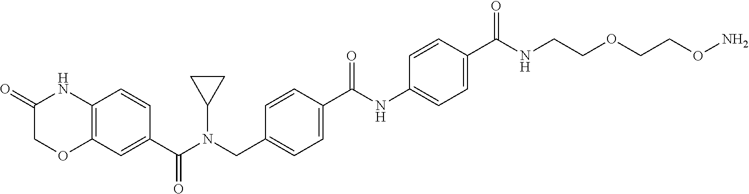 [SMILES: NOCCOCCNC(=O)c1ccc(NC(=O)c2ccc(CN(C(=O)c3ccc4c(c3)OCC(=O)N4)C3CC3)cc2)cc1] | 701.65 |
| | O═C(C1═CC═C(NC(C2═CC═C(CN(C3CC3)C(C4═CC(OCC(N5)═O)═C5C═C4)═O)C═C2)═O)C═C1)NCCOCCON | |
| 177 | 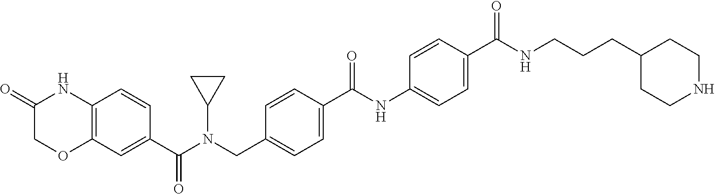 [SMILES: O=C1COc2cc(C(=O)N(Cc3ccc(C(=O)Nc4ccc(C(=O)NCCCC5CCNCC5)cc4)cc3)C3CC3)ccc2N1] | 723.74 |
| | O═C(C1═CC═C(NC(C2═CC═C(CN(C3CC3)C(C4═CC(OCC(N5)═O)═C5C═C4)═O)C═C2)═O)C═C1)NCCCC6CCNCC6 | |
| 178 | 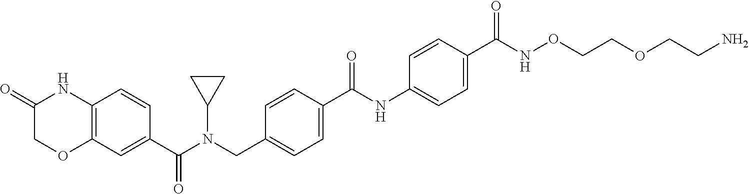 [SMILES: NCCOCCONC(=O)c1ccc(NC(=O)c2ccc(CN(C(=O)c3ccc4c(c3)OCC(=O)N4)C3CC3)cc2)cc1] | 701.65 |
| | O═C(C1═CC═C(NC(C2═CC═C(CN(C3CC3)C(C4═CC(OCC(N5)═O)═C5C═C4)═O)C═C2)═O)C═C1)NOCCOCCN | |
| 179 | 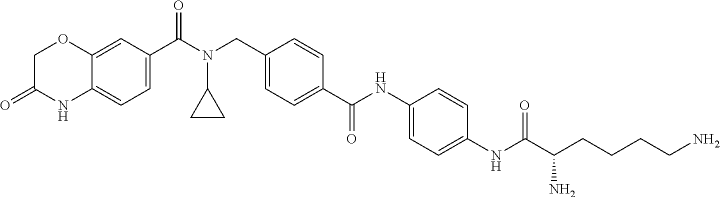 [SMILES: NCCCC[C@H](N)C(=O)Nc1ccc(NC(=O)c2ccc(CN(C(=O)c3ccc4c(c3)OCC(=O)N4)C3CC3)cc2)cc1] | 812.71 |
| | O═C(C1═CC═C2C(OCC(N2)═O)═C1)N(CC3═CC═C(C═C3)C(NC4═CC═C(C═C4)NC[C@H](CCCCN)N)═O)═O)C5CC5 | |
| 180 | 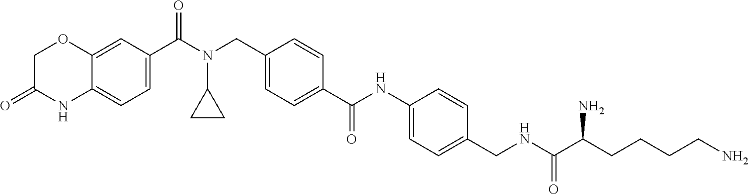 [SMILES: NCCCC[C@H](N)C(=O)NCc1ccc(NC(=O)c2ccc(CN(C(=O)c3ccc4c(c3)OCC(=O)N4)C3CC3)cc2)cc1] | 826.74 |
| | O═C(N(C1CC1)CC2═CC═C(C(NC3═CC═C(C═C3)CNC([C@@H](N)CCCCN)═O)═O)C═C2)C4═CC═C5C(OCC(N5)═O)═C4 | |
| 181 | 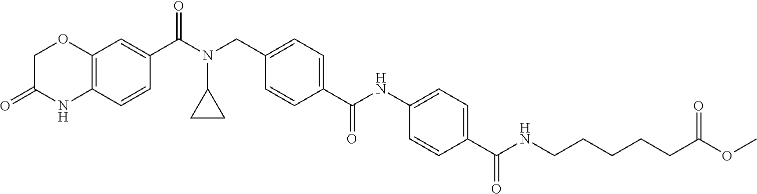 [SMILES: COC(=O)CCCCCNC(=O)c1ccc(NC(=O)c2ccc(CN(C(=O)c3ccc4c(c3)OCC(=O)N4)C3CC3)cc2)cc1] | 612.67 |
| | O═C(C1═CC═C(NC(C2═CC═C(CN(C3CC3)C(C4═CC(OCC(N5)═O)═C5C═C4)═O)C═C2)═O)C═C1)NCCCCCC(OC)═O | |
| 182 | 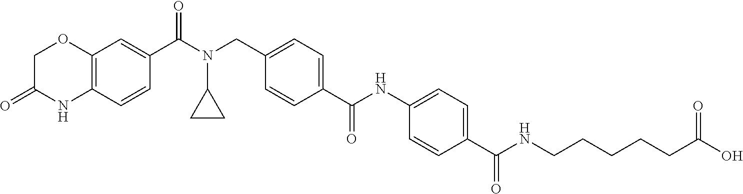 [SMILES: O=C(O)CCCCCNC(=O)c1ccc(NC(=O)c2ccc(CN(C(=O)c3ccc4c(c3)OCC(=O)N4)C3CC3)cc2)cc1] | 598.65 |
| | O═C(C1═CC═C(NC(C2═CC═C(CN(C3CC3)C(C4═CC(OCC(N5)═O)═C5C═C4)═O)C═C2)═O)C═C1)NCCCCCC(O)═O | |
| 183 | 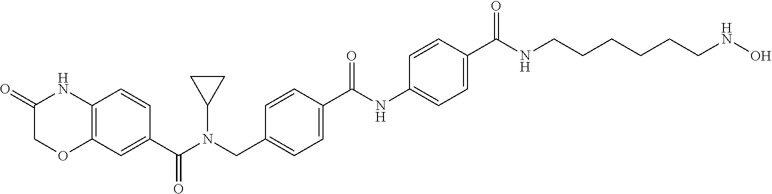 [SMILES: O=C1COc2cc(C(=O)N(Cc3ccc(C(=O)Nc4ccc(C(=O)NCCCCCCNO)cc4)cc3)C3CC3)ccc2N1] | 636.14 |
| | O═C(C1═CC═C(NC(C2═CC═C(CN(C3CC3)C(C4═CC(OCC(N5)═O)═C5C═C4)═O)C═C2)═O)C═C1)NCCCCCCNO | |
| 184 | 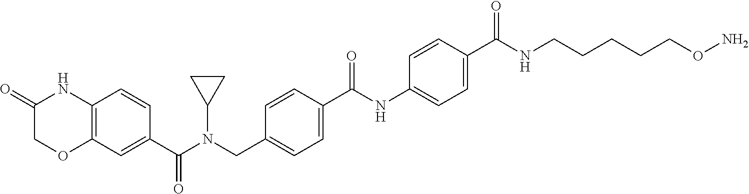 [SMILES: NOCCCCCNC(=O)c1ccc(NC(=O)c2ccc(CN(C(=O)c3ccc4c(c3)OCC(=O)N4)C3CC3)cc2)cc1] | 622.11 |
| | NOCCCCCNC(C1═CC═C(NC(C2═CC═C(CN(C3CC3)C(C4═CC(OCC(N5)═O)═C5C═C4)═O)C═C2)═O)C═C1)═O | |
| 185 | 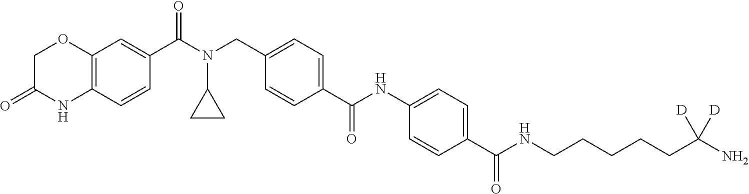 [SMILES: [2H]C([2H])(N)CCCCCNC(=O)c1ccc(NC(=O)c2ccc(CN(C(=O)c3ccc4c(c3)OCC(=O)N4)C3CC3)cc2)cc1] | 699.71 |
| | NC([2H])[2H])CCCCCNC(C1═CC═C(NC(C2═CC═C(CN(C3CC3)C(C4═CC(OCC(N5)═O)}═CSC═(4)}═O)C═C2)═O)C═C1)═O | |
| 186 | 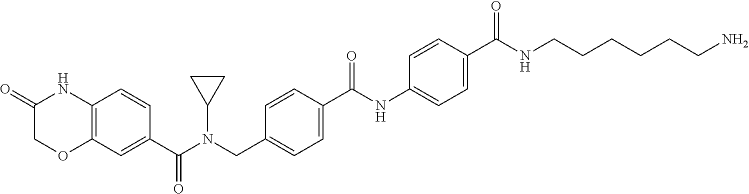 [SMILES: NCCCCCCNC(=O)c1ccc(NC(=O)c2ccc(CN(C(=O)c3ccc4c(c3)OCC(=O)N4)C3CC3)cc2)cc1] | 697.7 |
| | NCCCCCCNC(C1═CC═C(NC(C2═CC═C(CN(C3CC3)C(C4═CC(OCC(N5)═O)═C5C═C4)═O)C═C2)═O)C═C1)═O | |
| 187 | 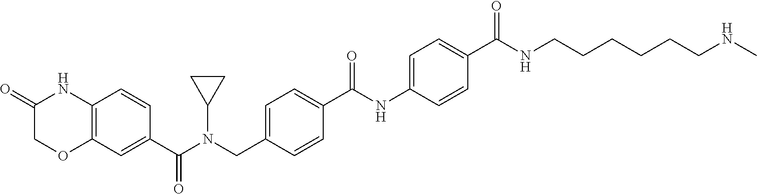 [SMILES: CNCCCCCCNC(=O)c1ccc(NC(=O)c2ccc(CN(C(=O)c3ccc4c(c3)OCC(=O)N4)C3CC3)cc2)cc1] | 711.73 |
| | O═C1NC(C═CC(C(N(CC2═CC═C(C(NC3═CC═C(C(NCCCCCCNC)═O)C═C3)═O)C═C2)C4CC4)═O)═C5)═C5OC1 | |
| 188 | 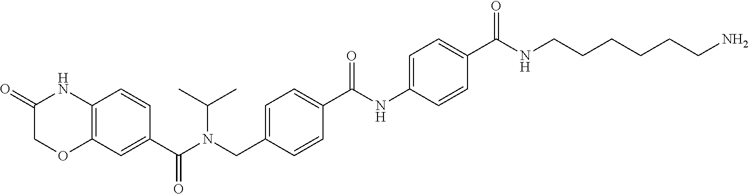 [SMILES: CC(C)N(Cc1ccc(C(=O)Nc2ccc(C(=O)NCCCCCCN)cc2)cc1)C(=O)c1ccc2c(c1)OCC(=O)N2] | 699.72 |
| | CC(N(CC1═CC═C(C(NC2═CC═C(C(NCCCCCCN)═O)C═C2)═O)C═C1)C(C(C═C3)═CCA═C3NC(CO4)═O)═O)C | |
| 189 | 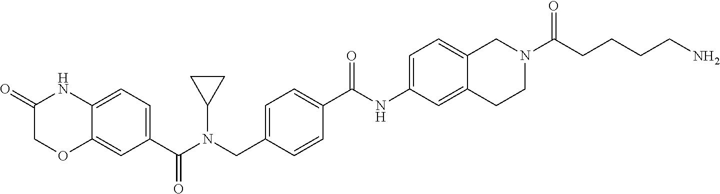 [SMILES: NCCCCC(=O)N1CCc2cc(NC(=O)c3ccc(CN(C(=O)c4ccc5c(c4)OCC(=O)N5)C4CC4)cc3)ccc2C1] | 709.71 |
| | O═C(N(C1CC1)CC2═CC═C(C(NC3═CC═C(CN4C(CCCCN)═O)C(CC4)═C3)═O)C═C2)C5═CC═C6C(OCC(N6)═O)═C5 | |
| 190 | 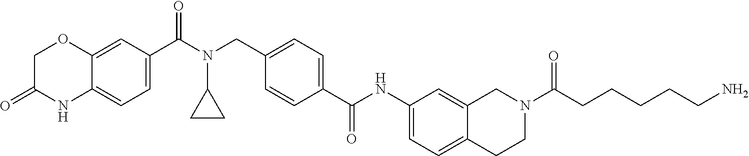 [SMILES: NCCCCCC(=O)N1CCc2ccc(NC(=O)c3ccc(CN(C(=O)c4ccc5c(c4)OCC(=O)N5)C4CC4)cc3)cc2C1] | 723.74 |
| | O═C(N(C1CC1)CC2═CC═C(C(NC3═CC═C(CCN(C(CCCCCN)═O)C4)C4═C3)═O)C═C2)C5═CC═C6C(OCC(N6)═O)═C5 | |
| 191 | 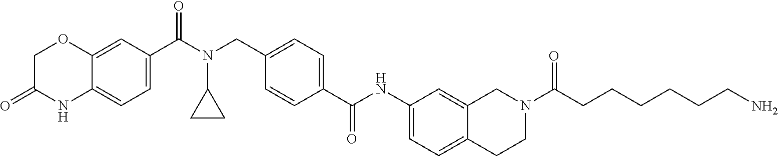 [SMILES: NCCCCCCC(=O)N1CCc2ccc(NC(=O)c3ccc(CN(C(=O)c4ccc5c(c4)OCC(=O)N5)C4CC4)cc3)cc2C1] | 737.76 |
| | O═C(N(C1CC1)CC2═CC═C(C(NC3═CC═C(CCN(C(CCCCCCN)═O)(4)C4═C3)═O)C═C2)C5═CC═C6C(OCC(N6)═O)═C5 | |
| 192 | 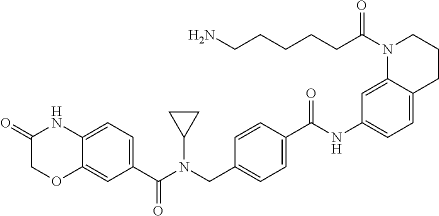 [SMILES: NCCCCCC(=O)N1CCCc2ccc(NC(=O)c3ccc(CN(C(=O)c4ccc5c(c4)OCC(=O)N5)C4CC4)cc3)cc21] | 723.74 |
| | O═C(N(C1CC1)CC2═CC═C(C(NC3═CC═C(CCCN4C(CCCCCN)═O)C4═C3)═O)C═C2)C5═CC═C6C(OCC(N6)═O)═C5 | |
| 193 | 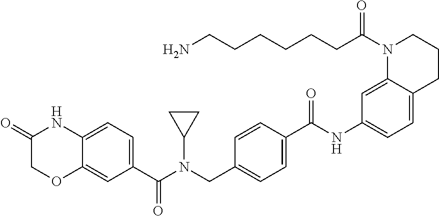 [SMILES: NCCCCCCC(=O)N1CCCc2ccc(NC(=O)c3ccc(CN(C(=O)c4ccc5c(c4)OCC(=O)N5)C4CC4)cc3)cc21] | 737.76 |
| | O═C(N(C1CC1)CC2═CC═C(C(NC3═CC═C(CCCN4C(CCCCCCN)═O)C4═C3)═O)C═C2)C5═CC═C6C(OCC(N6)═O)═C5 | |
| 194 | 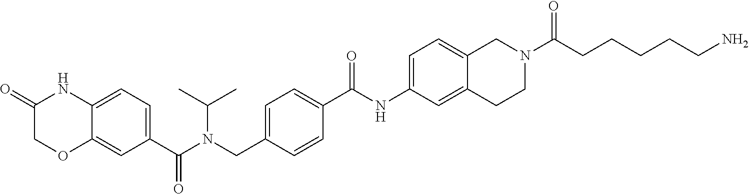 [SMILES: CC(C)N(Cc1ccc(C(=O)Nc2ccc3c(c2)CCN(C(=O)CCCCCN)C3)cc1)C(=O)c1ccc2c(c1)OCC(=O)N2] | 725.75 |
| | O═C(N(C(C)C)CC1═CC═C(C(NC2═CC═C(CN3C(CCCCCN)═O)C(CC3)═C2)═O)C═C1)C4═CC═C5C(OCC(N5)═O)═C4 | |

* Some of the molecular weights in the table are recorded as salt forms, e.g., trifluoroacetic acd (TFA). Some of the molecular weights are of compounds prepared in their neutral form.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography and/or recrystallization or by the forming diastereomers and separation thereof (Jean Jacques, Andre Collet, Samuel H. Wilen, “Enantiomers, Racemates and Resolutions”, John Wiley And Sons, Inc., 1981). Stereoisomers may also be obtained by stereoselective synthesis using synthetic methods known in the art. In some embodiments, the compounds disclosed herein are enantiomers having an enantiomeric excess (% ee) of at least about 50%, about 60%, about 70%, about 80%, about 90%, about 95%, about 98%, or about 99.5%. In some embodiments, the compounds disclosed herein are disateremores having a diatereomeric excess (% de) of at least about 50%, about 60%, about 70%, about 80%, about 90%, about 95%, about 98%, or about 99.5%. In some embodiments, the compounds disclosed herein are present as enantiomeric or diastereomeric mixtures.

The methods and compositions described herein include the use of amorphous forms as well as crystalline forms (also known as polymorphs). The compounds described herein may be in the form of pharmaceutically acceptable salts. Active metabolites of these compounds having the same type of activity are included in the scope of the present disclosure.

In some embodiments, the compounds described herein may be formed as, and/or used as, pharmaceutically acceptable salts. The type of pharmaceutical acceptable salts, include, but are not limited to: (l) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4methylenebis-3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like

In some embodiments, the compounds and salts described herein include isotopically-labeled compounds. In general, isotopically-labeled compounds are identical to those recited in the various formulae and structures presented herein, but for the fact that one or more atoms are replaced by an atom having an atomic mass or mass number different from the atomic mass or mass number most common in nature. Examples of isotopes that can be incorporated into the present compounds include isotopes of hydrogen, carbon, nitrogen, oxygen, fluorine and chlorine, for example, 2H, 3H, 13 C, 14C, 15N, 180, 17O, 35S, 18F, 36Cl, respectively. Certain isotopically-labeled compounds described herein, for example those into which radioactive isotopes such as 3H and 14C are incorporated, are useful in drug and/or substrate tissue distribution assays. Further, substitution with isotopes such as deuterium, i.e., 2H, can afford certain therapeutic advantages resulting from greater metabolic stability, such as, for example, increased in vivo half-life or reduced dosage requirements.

In some embodiments, the compounds or salts described herein is a prodrug. In some embodiments, the compounds described herein may be formed as, and/or used as, prodrugs. A “prodrug” refers to an agent that is converted into the parent drug in vivo. Prodrugs are often useful because, in some situations, they may be easier to administer than the parent drug. They may, for instance, be bioavailable by oral administration whereas the parentis not. The prodrug may also have improved solubility in pharmaceutical compositions over the parent drug. Examples, without limitation, of a prodrug would be a compound described herein, which is administered as an ester or an amide (the “prodrug”) to facilitate transmittal across a cell membrane where water solubility is detrimental to mobility but which then is metabolically hydrolyzed to the carboxylic acid and/or amine, the active entity, once inside the cell where water-solubility is beneficial. A further example of a prodrug might be a short peptide (polyamino acid) or amino acid (natural or unnatural) bonded to an acid group or an amine group where the peptide or amino acid is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. In some embodiments, by virtue of knowledge of pharmacodynamic processes and drug metabolism in vivo, once a pharmaceutically active compound is determined, prodrugs of the compound are designed. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

In some embodiments, the compounds disclosed herein bind to NSD2 and exhibit a dissociation constant (Kd) ranging from about 1 nM to about 500 nM, from about 1 nM to about 400 nM, from about 1 nM to about 300 nM, from about 1 nM to about 200 nM, from about 1 nM to about 100 nM, from about 1 nM to about 75 nM, from about 1 nM to about 50 nM, from about 1 nM to about 25 nM, or from about 1 nM to about 10 nM. In some embodiments, the disclosed compounds exhibit a Kd of less than about 500 nM, about 400 nM, about 300 nM, about 200 nM, about 100 nM, about 75 nM, about 50 nM, about 25 nM, or less than about 10 nM.

In some embodiments, the compounds disclosed herein degrade enzyme NSD2 in cells at concentrations ranging from about 1 microM to about 10 μM (i.e., at a concentration of less than about 10, about 9, about 8, about 7, about 6, about 5, about 4, about 3, about 2, or about 1 μM). In some embodiments, the compounds disclosed herein are present at a concentration that is at least 10 μM. At such concentrations the compound degrades enzyme NSD2 in such a manner that less than about 100%, about 75%, about 50%, about 25%, or less than about 10% of the enzyme remains intact after a certain time period of exposure. In some embodiments, the disclosed compounds degrade enzyme NSD2 and exhibit a DC50 (compound concentration at which 50% of the NSD2 protein is degraded) ranging from about 0.1 μM to about 5 μM, from about 0.1 μM to about 4 μM, from about 0.1 μM to about 3 μM, from about 0.1 μM to about 2 μM, from about 0.1 μM to about 1 μM, from about 0.1 μM to about 0.5 μM and from about 0.1 μM to about 0.25 μM. In some embodiments, the disclosed compounds degrade enzyme NSD2 and exhibit a DC50 (compound concentration at which 50% of the NSD2 protein is degraded) of less than about 5 μM, about 4 μM, about 3 μM, about 2 μM, about 1 μM, about 0.75 μM, about 0.50 μM, about 0.25 μM, or less than about 0.1 μM.

### III. Pharmaceutical Compositions

In certain embodiments, compounds, prodrugs or salts of Formulae (I), (II), and/or (III) disclosed herein, are combined with one or more additional agents to form pharmaceutical compositions. In some embodiments, compounds of Formulae (I), (II), and/or (III) are already in the form of a prodrug. Pharmaceutical compositions may be formulated in a conventional manner using one or more physiologically acceptable carriers including excipients and auxiliaries, which facilitate processing of the active compounds into preparations, which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound or salt or prodrug of Formulae (I), (II) and/or (III) with any suitable substituents and functional groups disclosed herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds or salts of Formula (I) with any suitable substituents and functional groups disclosed herein, can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include a compound of Formula (I), (II) and/or (III) with any suitable substituents and functional groups disclosed herein, can be formulated into any suitable dosage form, including but not limited to, aqueous oral dispersions, liquids, gels, syrups, elixirs, slurries, suspensions, aerosols, fast melt formulations, effervescent formulations, lyophilized formulations, tablets, powders, pills, dragees, and capsules.

One may administer the compounds and/or compositions in a local rather than systemic manner, for example, via injection of the compound directly into an organ or tissue, often in a depot preparation or sustained release formulation. Such long acting formulations may be administered by implantation (for example subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation, or in the form of an intermediate release formulation.

Pharmaceutical compositions including a compound described herein may be manufactured in a conventional manner, such as, by way of example only, by means of conventional mixing, dissolving granulating, dragee-making, levigating, emulsifying, encapsulating, entrapping or compression processes.

The pharmaceutical compositions will include at least one compound of Formula (I) disclosed herein, as an active ingredient in free-acid or free-base form, or in a pharmaceutically acceptable salt form.

In some embodiments, compositions provided herein may also include one or more preservatives to inhibit microbial activity. Suitable preservatives include quaternary ammonium compounds such as benzalkonium chloride, cetyltrimethylammonium bromide and cetylpyridinium chloride.

In some embodiments, the pharmaceutical solid dosage forms described herein can include a compound of Formulae (I), (II) and/or (III) and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, ortragacanth, sodium starchglycolate, bentonite, a natural sponge, a surfactant, a resin such as a cation-exchange resin, citrus pulp, sodium lauryl sulfate, sodium lauryl sulfate in combination starch, and the like.

Suitable binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10@), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g, Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 5400 to about 7000, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

There is considerable overlap between additives used in the solid dosage forms described herein. Thus, the above-listed additives should be taken as merely exemplary, and not limiting, of the types of additives that can be included in solid dosage forms of the pharmaceutical compositions described herein.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002).

The pharmaceutical compositions described herein may include sweetening agents such as, but not limited to, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

Potential excipients for intranasal formulations include formulations solutions in saline, employing benzyl alcohol or other suitable preservatives, fluorocarbons, and/or other solubilizing or dispersing agents. See, for example, Ansel, H. C. et al., Pharmaceutical Dosage Forms and Drug Delivery Systems, Sixth Ed. (1995). Preferably, these compositions and formulations are prepared with suitable nontoxic pharmaceutically acceptable ingredients. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal formulations that include compounds described herein may be administered using a variety of formulations, which include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound is provided essentially throughout. Buccal drug delivery avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, virtually any such carrier can be used, so long as the desired drug release profile is not compromised, and the carrier is compatible with the compounds described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

Transdermal formulations described herein may incorporate certain pharmaceutically acceptable excipients, which are conventional in the art. In some embodiments, formulations suitable for transdermal administration of compounds described herein may employ transdermal delivery devices and transdermal delivery patches and can be lipophilic emulsions or buffered, aqueous solutions, dissolved and/or dispersed in a polymer or an adhesive.

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and are formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the compounds of Formulae (I), (II), and/or (III) disclosed herein are combined with other therapeutic agents, such as other anti-cancer agents, anti-allergic agents, anti-nausea agents (or anti-emetics), pain relievers, cytoprotective agents, and combinations thereof.

In another embodiment, the compounds of Formulae (I), (II) and/or (III) disclosed herein are combined with another therapeutic agent capable of inhibiting BRAF, MEK, CDK4/6, SHP-2, HDAC, EGFR, MET, mTOR, PI3K or AKT, or a combination thereof.

Generally, an agent, such as a compound of Formula (I) disclosed herein, is administered in an amount effective for treating the disease or disorder (i.e., a therapeutically effective amount). Thus, a therapeutically effective amount can be an amount that is capable of at least partially treating, preventing or reversing a disease or disorder. The dose required to obtain an effective amount may vary depending on the agent, formulation, disease or disorder, and individual to whom the agent is administered.

Determination of effective amounts may also involve in vitro assays in which varying doses of agent are administered to cells in culture and the concentration of agent effective for ameliorating some or all symptoms is determined in order to calculate the concentration required in vivo. Effective amounts may also be based in in vivo animal studies.

An agent can be administered prior to, concurrently with and subsequent to the appearance of symptoms of a disease or disorder. In some embodiments, an agent is administered to a subject with a family history of the disease or disorder, or who has a phenotype that may indicate a predisposition to a disease or disorder, or who has a genotype which predisposes the subject to the disease or disorder.

In some embodiments, the compositions described herein are provided as pharmaceutical and/or therapeutic compositions. The pharmaceutical and/or therapeutic compositions of the present disclosure can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g, intrathecal or intraventricular, administration. Compositions and formulations for topical administration can include transdermal patches, ointments, lotions, creams, gels, drops, suppositories, sprays, liquids and powders. Conventional carriers; aqueous, powder, or oily bases; thickeners; and the like can be necessary or desirable. Compositions and formulations for oral administration include powders or granules, suspensions or solutions in water or non-aqueous media, capsules, sachets or tablets. Thickeners, flavoring agents, diluents, emulsifiers, dispersing aids or binders can be desirable. Compositions and formulations for parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. Pharmaceutical and/or therapeutic compositions of the present disclosure include, but are not limited to, solutions, emulsions, and liposome containing formulations. These compositions can be generated from a variety of components that include, but are not limited to, preformed liquids, self-emulsifying solids and self-emulsifying semisolids.

The pharmaceutical and/or therapeutic formulations, which can conveniently be presented in unit dosage form, can be prepared according to conventional techniques well known in the pharmaceutical/nutriceutical industries. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. The compositions of the present disclosure can be formulated into any of many possible dosage forms such as, but not limited to, tablets, capsules, liquid syrups, soft gels, suppositories, and enemas. The compositions of the present disclosure can also be formulated as suspensions in aqueous, non-aqueous, oil-based, or mixed media. Suspensions can further contain substances that increase the viscosity of the suspension including, for example, sodium carboxymethylcellulose, sorbitol and/or dextran. The suspension can also contain stabilizers. In one embodiment of the present disclosure the pharmaceutical compositions can be formulated and used as foams. Pharmaceutical foams include formulations such as, but not limited to, emulsions, microemulsions, creams, jellies and liposomes. While basically similar in nature these formulations vary in the components and the consistency of the final product.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Dosing and administration regimes are tailored by the clinician, or others skilled in the pharmacological arts, based upon well-known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, the compounds are administered to a subject at a dose of about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone. Dosing may be once per day or multiple times per day for one or more consecutive days.

### IV. Methods of Treatment

The present disclosure provides compounds and methods for binding to and/or inhibiting the activity of the NSD2 enzyme. In some embodiments, the present disclosure provides compounds and methods that degrade the NSD2 enzyme.

Inhibition of NSD2 activity may be assessed and demonstrated by a wide variety of ways known in the art. Non-limiting examples include measure (a) a direct decrease in NSD2 activity; (b) a decrease in cell proliferation and/or cell viability; (c) an increase in cell differentiation; (d) a decrease in the levels of downstream targets of NSD2 activity; and (e) decrease in tumor volume and/or tumor volume growth rate. Kits and commercially available assays can be utilized for determining one or more of the above.

Binding of compounds disclosed herein to the NSD2 enzyme can be determined using known methods in the arts, such as, but not limited to Surface Plasmon Resonance (SPR).

The disclosure provides compounds and methods for treating a subject suffering from a disease, comprising administering a compound, prodrug or salt described herein, for example, a compound, prodrug or salt of Formulae (I), (II) and/or (III) disclosed herein, to the subject. In some embodiments, the disease is selected from a disease associated with NSD2 expression (e.g., aberrant expression, overexpression, etc.) and/or activity (e.g., cancer). In certain embodiments, the disease is mediated by NSD2 activity and/or expression (e.g., aberrant expression, over expression, etc.). In some embodiments, the disease or condition is treatable by inhibition of and/or degradarion of the NDS2 enzyme. In some embodiments, the method comprises treating a disease or condition that is treatable by inhibition of NDS2 by administering to a subject in need thereof a therapeutically effective amount of a compound, prodrug, or a salt thereof of Formulae (I), (II), and/or (III) or a pharmaceutical composition as disclosed herein.

In some embodiments, the disclosure provides a method for treating cancer in a subject, comprising administering a compound, prodrug or salt described herein, for example, a compound, prodrug or salt of Formulae (I), (II), and/or (III) disclosed herein, to the subject. In some embodiments, the cancer is mediated by a NSD2 expression (e.g., aberrant expression, overexpression, etc.) and/or activity.

In certain embodiments, the disclosure provides method of treating a disease in a subject, wherein the method comprises determining if the subject has an NSD2-mediated condition (e.g., cancer) and administering to the subject a therapeutically effective dose of a compound, prodrug or salt described herein, for example, a compound, prodrug or salt of Formulae (I), (II), and/or (III) as disclosed herein.

Determining whether a tumor or cancer expresses (e.g., overexpresses, aberrantly expresses, etc.) NSD2 can be undertaken by assessing the nucleotide sequence encoding NSD2 or by assessing the amino acid sequence of NSD2. Methods for detecting an NSD2 nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. Methods for detecting an NSD2 protein are known by those of skill in the art. These methods include, but are not limited to, detection using a binding agent, e.g., an antibody, specific for NSD2, protein electrophoresis and Western blotting, and direct peptide sequencing.

Methods for determining whether a tumor or cancer expresses (e.g., overexpresses, aberrantly expresses, etc.) NSD2 or is mediated by NSD2 activity can use a variety of samples. In some embodiments, the sample is taken from a subject having a tumor or cancer. In some embodiments, the sample is taken from a subject having a cancer or tumor. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

In certain embodiments, the disclosure provides a method of inhibiting NSD2 activity in a sample, comprising administering the compound or salt described herein to said sample comprising NSD2.

The disclosure provides methods for treating a disease by administering a compound, prodrug or salt of Formulae (I), (II), and/or (III) disclosed herein, to a subject suffering from the disease, wherein the compound binds to NSD2 and/or inhibits NSD2 activity. In some embodiments, the compound covalently binds to NSD2. In some embodiments, the compound noncovalently binds to NSD2. In some embodiments, the compound degrades the NSD2 enzyme.

The disclosure also relates to a method of treating a hyperproliferative disorder in a mammal that comprises administering to the mammal a therapeutically effective amount of a compound, prodrug, or salt of Formulae (I), (II), and/or (III) with any suitable substituents and functional groups disclosed herein. In some embodiments, the method relates to the treatment of cancer such as acute myeloid leukemia, cancer in adolescents, adrenocortical carcinoma childhood, AIDS-related cancers, e.g., Lymphoma and Kaposi's Sarcoma, anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinomain situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, the method relates to the treatment of a non-cancerous hyperproliferative disorder such as benign hyperplasia of the skin, e.g., psoriasis, restenosis, or prostate, e.g., benign prostatic hypertrophy (BPH). In some cases, the method relates to the treatment of leukemia, hematologic malignancy, solid tumor cancer, prostate cancer, e.g., castration-resistant prostate cancer, breast cancer, Ewing's sarcoma, bone sarcoma, primary bone sarcoma, T-cell prolymphocyte leukemia, glioma, glioblastoma, liver cancer, e.g., hepatocellular carcinoma, or diabetes.

Subjects that can be treated with compounds of Formulae (I), (II), and/or (III) disclosed herein, or pharmaceutically acceptable salt, ester, prodrug, stereoisomer, or enantiomer of the compounds, according to the methods of this disclosure include, for example, subjects that have been diagnosed as having acute myeloid leukemia, acute myeloid leukemia, cancer in adolescents, adrenocortical carcinoma childhood, AIDS-related cancers, e.g., Lymphoma and Kaposi's Sarcoma, anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, Viral-Induced cancer, leukemia, hematologic malignancy, solid tumor cancer, prostate cancer, castration-resistant prostate cancer, breast cancer, Ewing's sarcoma, bone sarcoma, primary bone sarcoma, T-cell prolymphocyte leukemia, glioma, glioblastoma, hepatocellular carcinoma, liver cancer, or diabetes. In some embodiments, subjects that are treated with the compounds of the disclosure include subjects that have been diagnosed as having a non-cancerous hyperproliferative disorder such as benign hyperplasia of the skin, e.g., psoriasis, restenosis, or prostate, e.g., benign prostatic hypertrophy (BPH).

The disclosure further provides methods of inhibiting NSD2 activity, by contacting the NSD2 with an effective amount of a compound, prodrug or salt of Formulae (I), (II) and/or (III) disclosed herein (e.g., by contacting a cell, tissue, or organ that expresses NSD2). In some embodiments, the disclosure provides methods of inhibiting NSD2 activity in a subject including but not limited to rodents and mammals, e.g., humans, by administering to the subject an effective amount of a compound of Formulae (I), (II) and/or (III) disclosed herein. Thus, the present disclosure is directed to methods of inhibiting NSD2 activity in a in vitro and in vivo testing environment, which a skilled artisan would be familiar with. In some embodiments, the percentage of inhibition of the NSD2 enzyme in vitro and/or in vivo is at least 25%, 30%, 40%, 50%, 60%, 70%, 80%, or 90%.

For example, in some embodiments, the disclosure provides methods of inhibiting NSD2 activity in a cell by contacting the cell with an amount of a compound as disclosed herein sufficient to inhibit the activity. In some embodiments, the disclosure provides methods of inhibiting NSD2 activity in a tissue by contacting the tissue with an amount of a compound, prodrug or salt of Formulae (I), (II) and/or (III) as disclosed herein, sufficient to inhibit the NSD2 activity in the tissue. In some embodiments, the disclosure provides methods of inhibiting NSD2 activity in an organism (e.g., mammal, human, etc.) by contacting the organism with an amount of a compound, prodrug or salt of Formulae (I), (II) and/or (III) as disclosed herein, sufficient to inhibit the NSD2 activity in the organism.

In some embodiments, the methods disclosed herein are directed to compounds that are able to degrade the NSD2 enzyme. For example, methods of degrading the NSD2 enzyme comprises contacting the NSD2 with an effective amount of a compound, prodrug or salt of Formulae (I), (II) and/or (III) disclosed herein (e.g., by contacting a cell, tissue, or organ that expresses NSD2). In some embodiments, the disclosure provides methods of degrading the NSD2 enzyme in a subject including but not limited to rodents and mammals, e.g., humans, by administering to the subject an effective amount of a compound of Formulae (I), (II) and/or (III) disclosed herein. In some embodiments, the percentage of degradation of the NSD2 enzyme is at least 25%, 30%, 40%, 50%, 60%, 70%, 80%, or 90%.

The compositions containing the compounds or salts thereof described herein can be administered for prophylactic and/or therapeutic treatments. In therapeutic applications, the compositions are administered to a patient already suffering from a disease, in an amount sufficient to cure or at least partially arrest the symptoms of the disease. Amounts effective for this use will depend on the severity and course of the disease, previous therapy, the patient's health status, weight, and response to the drugs, and the judgment of the treating clinician.

In prophylactic applications, compositions containing the compounds or salts thereof described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating clinician.

The amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### EXAMPLES

### Example 1

### General Procedures

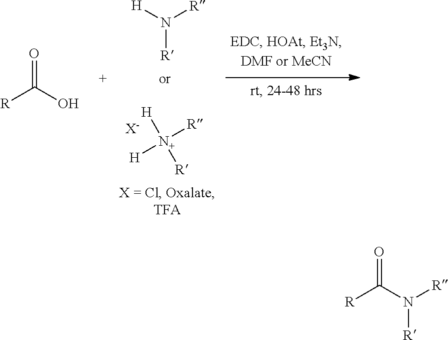

General Procedure A: For the Coupling of Aliphatic Amines or Anilines with Achiral Carboxylic Acids

To a scintillation vial charged with a stirbar was added carboxylic acid (1 Eq), EDC (1.5-2 Eq), HOAt (1.5-2 Eq) and DMF or MeCN (0.5-4 mL). The mixture was left to stir at room temperature for 30 minutes. To the vial was then added amine/aniline (1-1.9 Eq) followed by triethylamine (3-4 Eq). The reaction was stirred at room temperature for 24 (aliphatic amines) or 48 (anilines) hours. See below for N-Boc deprotection (when relevant) and workup/purification. Following N-Boc deprotection and purification, all free aliphatic amine products were isolated as trifluoroacetate salts (unless otherwise noted). All final products were suspended in water, flash frozen and lyophilized to dryness.

Workup A: The reaction was diluted with distilled water and extracted 3 times with ethyl acetate. The combined organic layers were washed once with water, twice with saturated sodium bicarbonate, once with brine, then dried over sodium sulfate, filtered and concentrated in vacuo.

Workup B: The reaction was poured onto water (10× v/v w.r.t. DMF) and the precipitate collected by centrifugation. Supernatant was discarded. To the pellet was added 10 mL of water, and the pellet was re-suspended with sonication. The precipitate was again collected by centrifugation and the supernatant again discarded. The pellet briefly air-dried before being suspended in methanol/DCM and transferred to a scintillation vial where volatiles were removed in vacuo.

N-Boc Deprotection: To a scintillation vial containing a stirbar and crude reaction mixture or purified N-Boc-protected amine was added TFA in DCM (10 Eq, 20% v/v). The mixture was stirred overnight. Volatiles were removed in vacuo and residual TFA was removed by co-evaporation with methanol.

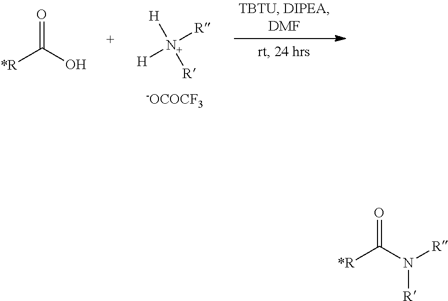

General Procedure B: For the Coupling of Aliphatic Amines with Chiral Carboxylic Acids

To a scintillation vial charged with a stirbar was added carboxylic acid (1.1 Eq), TBTU (1.3 Eq) and DMF (0.5-2 mL). The mixture was left to stir at room temperature for 30 minutes. To the vial was then added amine salt (1 Eq) followed by DIPEA (3.3 Eq). The reaction was stirred at room temperature for 24 hours. See above for N-Boc deprotection (when relevant) and workup/purification. Following N-Boc deprotection and purification, all free aliphatic amine products were isolated as trifluoroacetate salts (unless otherwise noted). All final products were suspended in water, flash frozen and lyophilized to dryness.

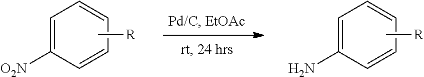

### General Procedure C: For the Reduction of Nitroarenes

To a round-bottom flask charged with a stirbar was added nitroarene (1 Eq) and ethyl acetate (5-24 mL), following which the reaction was evacuated and purged with nitrogen for three cycles. Pd/C (10% Wt) was added and the evacuation/refill cycle was repeated thrice more. A hydrogen balloon was equipped and the reaction was stirred at room temperature while monitoring by TLC. If complete consumption of starting material did not occur by 24 hours, the Pd/C was replenished and a second hydrogen balloon was equipped. Upon consumption of starting material, the reaction was filtered over celite and rinsed thoroughly with ethyl acetate. The filtrate was concentrated in vacuo. See individual reaction procedures for purification details.

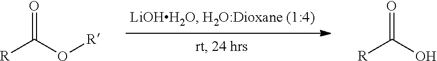

### General Procedure D: For the Hydrolysis of Esters

To a round-bottom flask charged with a stirbar was added ester (1 Eq) and THE or dioxane (4-5 mL). Next, a solution of lithium hydroxide hydrate (5 Eq) in water (1-2 mL) was added, and the reaction was stirred overnight with monitoring by TLC. Upon consumption of starting material the mixture was washed with 5 portions of ether (organics discarded) and the pH was adjusted to 2 with concentrated HCl. Upon addition of acid, a precipitate formed. The precipitate was collected by filtration, washed with copious amounts of water and air-dried to give product, which was used without further purification (unless otherwise noted).

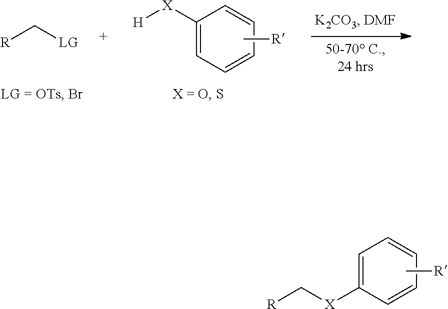

General Procedure E: For the Substitution of Alkyl Tosylates Bromides with Phenols Thiophenols

To a scintillation vial charged with a stirbar was added alkyl tosylate/bromide (1 Eq), DMF (3-5 mL), phenol/thiophenol (1.2-1.3 Eq) and potassium carbonate (1.5-3 Eq). The vial was heated to 50° C. (tosylates) or 70° C. (bromides) and allowed to stir overnight. The reaction was allowed to cool to room temperature and was quenched with water and extracted 3 times with ethyl acetate. The combined organic fractions were washed once with 1 M NaOH, three times with water, once with brine, dried over sodium sulfate, filtered, and concentrated. See individual reaction procedures for purification details.

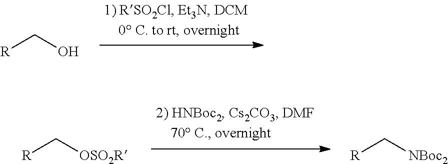

General Procedure F: For the Conversion of Alcohols to Sulfonate Esters then Displacement with Di-Tert-Butyl Iminodicarboxylate

(Step 1) To a round-bottom flask charged with a stirbar was added alcohol (1 Eq), DCM (5-15 mL) and triethylamine (1.8-2 Eq). The flask was cooled in an ice bath and sulfonyl chloride (1.4-1.5 Eq) was added dropwise. The reaction was allowed to come to room temperature with stirring overnight. The reaction was quenched with 1M HCl and extracted 3 times with DCM. The combined organic layers were washed once with water, once with saturated sodium bicarbonate, once with brine, dried over sodium sulfate, filtered and concentrated in vacuo.

(Step 2) To the sulfonate ester was then added di-tert-butyl iminodicarbonate (1.2 Eq), Cs₂CO₃ (1.5 Eq) and DMF (2.5-5 mL). The reaction was heated to 70° C. and stirred overnight. The reaction was then cooled and diluted with water. The aqueous solution was extracted three times with ethyl acetate, and the combined organic layers washed three times with water, once with brine, then dried over sodium sulfate, filtered and concentrated. See individual reaction procedures for purification details.

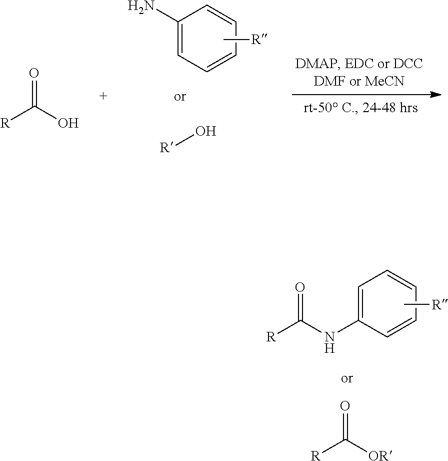

### General Procedure G: Alternative Method for the Coupling of Carboxylic Acids and Anilines Alcohols)

To a stirred solution of carboxylic acid (1 Eq) in DMF/MeCN (1-5 mL) was added DMAP (0.1-2 Eq) and aniline/alcohol (2-4 Eq). EDC/DCC (1.1-2 Eq) was added and the reaction was stirred at rt or 50° C. overnight. See individual procedures for workup/purification details.

### Example 2

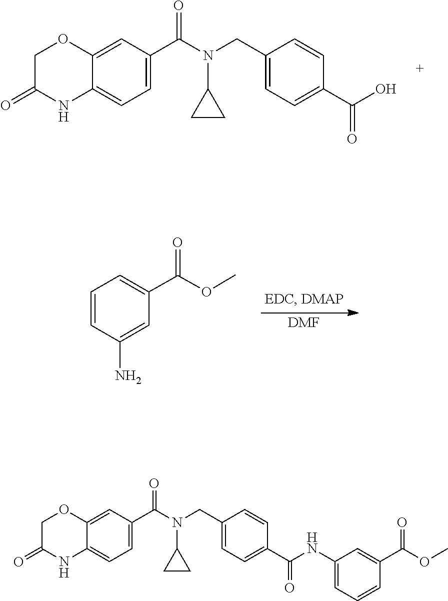

Prepared according to General Procedure G using 4-((N-cyclopropyl-3-oxo-3,4-dihydro-2H-benzo[b][1,4]oxazine-7-carboxamido)methyl)benzoic acid (200 mg, 1 Eq, 546 μmol), methyl 3-aminobenzoate (165 mg, 2 Eq, 1.09 mmol), DMAP (133 mg, 2 Eq, 1.09 mmol), EDC (209 mg, 2 Eq, 1.09 mmol), and DMF (2 mL) at 50° C. The clear orange reaction was cooled to room temperature and diluted with ethyl acetate and water. The phases were separated and the aqueous layer was extracted twice more with ethyl acetate. The organic layer was washed once with 1M KHSO₄, once with water, once with saturated sodium bicarbonate, once with brine, then dried over sodium sulfate and concentrated to an orange oil. The material was dissolved in DCM with methanol as needed and adsorbed onto Celite. Normal phase chromatography over silica gel (0-10% methanol in DCM) yielded methyl 3-(4-((N-cyclopropyl-3-oxo-3,4-dihydro-2H-benzo[b][1,4]oxazine-7-carboxamido)methyl)benzamido)benzoate (239 mg, 478 μmol, 87.6%) as a white solid. ¹H NMR (400 MHz, cdcl₃) δ 9.02 (s, 1H), 8.23 (t, J=1.9 Hz, 1H), 8.01 (ddd, J=8.1, 2.3, 1.1 Hz, 1H), 7.85-7.81 (m, 2H), 7.74 (dt, J=7.8, 1.3 Hz, 1H), 7.36 (t, J=8.0 Hz, 1H), 7.28 (d, J=7.9 Hz, 2H), 7.05 (d, J=1.7 Hz, 1H), 7.02 (dd, J=8.1, 1.8 Hz, 1H), 6.78 (d, J=8.0 Hz, 1H), 4.70 (s, 2H), 4.55 (s, 2H), 3.85 (s, 3H), 2.59 (tt, J=7.0, 3.9 Hz, 1H), 0.57 (d, J=6.8 Hz, 2H), 0.45 (s, 2H). LCMS (ESI, +ve mode) expected m/z for [C₂₈H₂₆N₃O₆⁺] [M+H] 500.2, found 500.1.

### Example 3

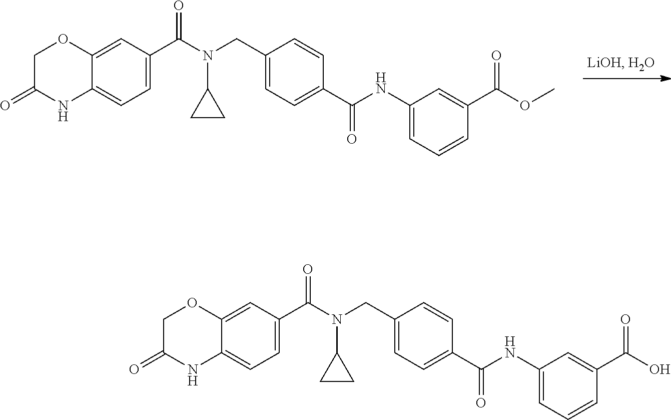 [SMILES: COC(=O)c1cccc(NC(=O)c2ccc(CN(C(=O)c3ccc4c(c3)OCC(=O)N4)C3CC3)cc2)c1 ; O=C1COc2cc(C(=O)N(Cc3ccc(C(=O)Nc4cccc(C(=O)O)c4)cc3)C3CC3)ccc2N1]

Prepared according to General Procedure D using methyl 3-(4-((N-cyclopropyl-3-oxo-3,4-dihydro-2H-benzo[b][1,4]oxazine-7-carboxamido)methyl)benzamido)benzoate (239 mg, 1 Eq, 478 μmol), THF (5 mL), lithium hydroxide hydrate (100 mg, 5 Eq, 2.39 mmol) and Water (1.25 mL) to give 3-(4-((N-cyclopropyl-3-oxo-3,4-dihydro-2H-benzo[b][1,4]oxazine-7-carboxamido)methyl)benzamido)benzoic acid (191.6 mg, 394.6 μmol, 82.5%) as a white solid. ¹H NMR (400 MHz, dmso) δ 10.87 (s, 1H), 10.40 (s, 1H), 8.41 (t, J=1.9 Hz, 1H), 8.02 (d, J=8.0 Hz, 1H), 7.96 (d, J=8.0 Hz, 2H), 7.66 (dt, J=7.7, 1.4 Hz, 1H), 7.52-7.33 (m, 3H), 7.17 (dd, J=8.0, 1.8 Hz, 1H), 7.13 (s, 1H), 6.92 (d, J=8.1 Hz, 1H), 4.70 (s, 2H), 4.60 (s, 2H), 2.78 (s, 1H), 0.53 (d, J=6.9 Hz, 2H), 0.46 (s, 2H). LCMS (ESI, +ve mode) expected m/z for [C₂₇H₂₄N₃O₆⁺] [M+H] 486.2, found 486.2.

### Example 4

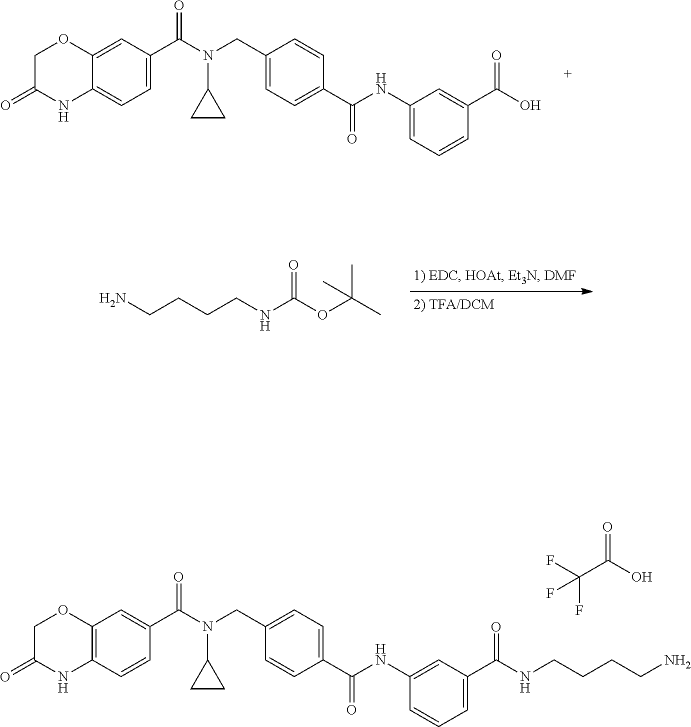 [SMILES: O=C1COc2cc(C(=O)N(Cc3ccc(C(=O)Nc4cccc(C(=O)O)c4)cc3)C3CC3)ccc2N1 ; CC(C)(C)OC(=O)NCCCCN ; NCCCCNC(=O)c1cccc(NC(=O)c2ccc(CN(C(=O)c3ccc4c(c3)OCC(=O)N4)C3CC3)cc2)c1 ; O=C(O)C(F)(F)F]

Prepared according to General Procedure A/Workup A using 3-(4-((N-cyclopropyl-3-oxo-3,4-dihydro-2H-benzo[b][1,4]oxazine-7-carboxamido)methyl)benzamido)benzoic acid (60.0 mg, 1 Eq, 124 μmol), EDC (35.5 mg, 1.5 Eq, 185 μmol), tert-butyl (4-aminobutyl)carbamate (34.9 mg, 1.5 Eq, 185 μmol), HOAt (25.2 mg, 1.5 Eq, 185 μmol), DMF (1 mL) and triethylamine (50.0 mg, 69 μL, 4 Eq, 494 μmol). Following N-Boc deprotection, the product was purified by reverse phase chromatography (10-100% methanol in water+0.1% TFA) to give N-(4-((3-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (3.66 mg, 5.47 μmol, 4.42%) as a red solid. ¹H NMR (400 MHz, cd₃od) δ 8.23 (t, J=1.8 Hz, 1H), 7.96 (d, J=8.2 Hz, 2H), 7.80 (dd, J=8.1, 1.1 Hz, 1H), 7.59 (d, J=8.1 Hz, 1H), 7.49 (s, 1H), 7.48-7.39 (m, 2H), 7.19 (d, J=8.2 Hz, 1H), 7.16 (s, 1H), 6.96 (d, J=8.1 Hz, 1H), 4.82 (s, 2H), 4.61 (s, 2H), 3.44 (t, J=6.3 Hz, 2H), 2.99 (t, J=6.8 Hz, 2H), 2.82 (s, 1H), 1.90-1.49 (m, 4H), 0.63 (s, 2H), 0.55 (s, 2H). LCMS (ESI, +ve mode) expected m/z for [C₃₁H₃₄N₅O₅⁺] [M+H] 556.3, found 556.2.

### Example 5

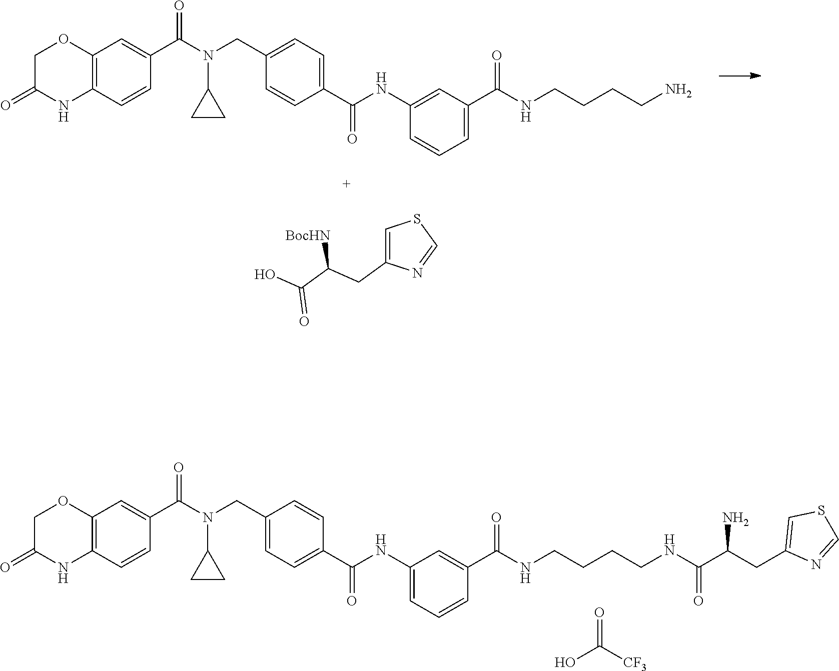 [SMILES: NCCCCNC(=O)c1cccc(NC(=O)c2ccc(CN(C(=O)c3ccc4c(c3)OCC(=O)N4)C3CC3)cc2)c1 ; CC(C)(C)OC(=O)N[C@@H](Cc1cscn1)C(=O)O ; N[C@@H](Cc1cscn1)C(=O)NCCCCNC(=O)c1cccc(NC(=O)c2ccc(CN(C(=O)c3ccc4c(c3)OCC(=O)N4)C3CC3)cc2)c1 ; O=C(O)C(F)(F)F]

Prepared according to General Procedure B/Workup A using Boc-3-(4-Thiazolyl)-L-alanine (8.9 mg, 1.1 Eq, 33 μmol), TBTU (12 mg, 1.3 Eq, 39 μmol), DMF (1 mL), N-(4-((3-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 30 μmol) and DIPEA (13 mg, 17 μL, 3.3 Eq, 99 μmol). Following N-Boc deprotection, the product was purified by reverse phase chromatography (10-100% methanol in water+0.1% TFA) to give (S)—N-(4-((3-((4-(2-amino-3-(thiazol-4-yl)propanamido)butyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5.83 mg, 7.08 μmol, 24%) as a white solid. ¹H NMR (400 MHz, cd₃od) δ 9.00 (d, J=1.9 Hz, 1H), 8.31-8.15 (m, 1H), 7.97 (d, J=8.2 Hz, 2H), 7.88-7.71 (m, 1H), 7.59 (d, J=8.0 Hz, 1H), 7.57-7.44 (m, 3H), 7.42 (d, J=1.9 Hz, 1H), 7.28-7.08 (m, 2H), 6.97 (d, J=8.0 Hz, 1H), 4.84 (s, 2H), 4.62 (s, 2H), 4.19 (dd, J=7.5, 5.9 Hz, 1H), 3.48-3.32 (m, 4H), 3.29-3.08 (m, 2H), 2.84 (s, 1H), 1.68-1.44 (m, 4H), 0.79-0.61 (m, 2H), 0.61-0.35 (m, 2H). LCMS (ESI, +ve mode) expected m/z for [C₃₇H₄₀N₇O₆S⁺] [M+H] 710.3, found 710.2.

### Example 6

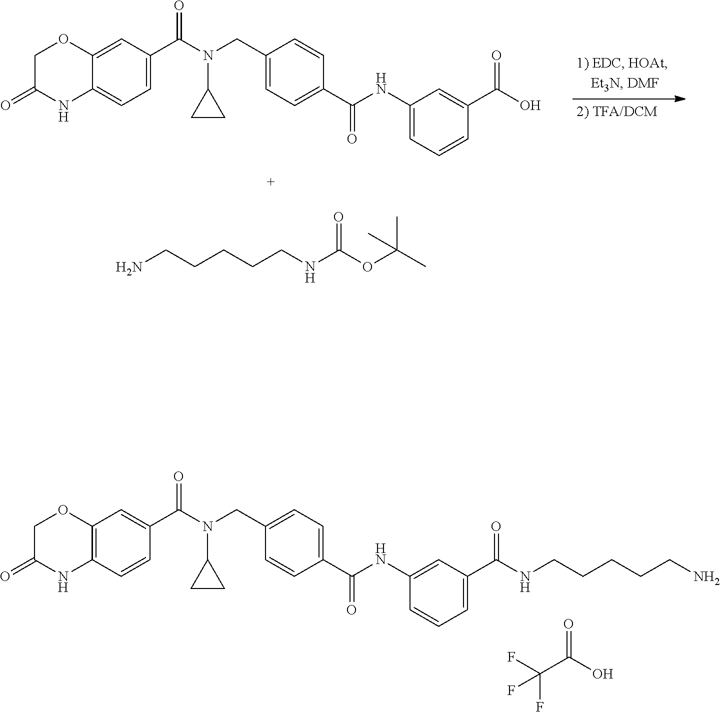 [SMILES: O=C1COc2cc(C(=O)N(Cc3ccc(C(=O)Nc4cccc(C(=O)O)c4)cc3)C3CC3)ccc2N1 ; CC(C)(C)OC(=O)NCCCCCN ; NCCCCCNC(=O)c1cccc(NC(=O)c2ccc(CN(C(=O)c3ccc4c(c3)OCC(=O)N4)C3CC3)cc2)c1 ; O=C(O)C(F)(F)F]

Prepared according to General Procedure A/Workup A using 3-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 154 μmol), HOAt (21.0 mg, 1.5 Eq, 154 μmol), DMF (1 mL), tert-butyl (5-aminopentyl)carbamate (31.3 mg, 1.5 Eq, 154 μmol) and triethylamine (41.7 mg, 57 μL, 4 Eq, 412 μmol). Following N-Boc deprotection, the product was purified by reverse phase chromatography (10-100% methanol in water+0.1% TFA) to give N-(4-((3-((5-aminopentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (27.49 mg, 40.21 μmol, 39.0%) as a white solid. ¹H NMR (400 MHz, cd₃od) δ 8.21 (t, J=1.7 Hz, 1H), 7.96 (d, J=8.2 Hz, 2H), 7.86-7.72 (m, 1H), 7.58 (d, J=8.1 Hz, 1H), 7.50 (s, 2H), 7.50-7.37 (m, 2H), 7.20 (d, J=8.2 Hz, 2H), 7.17 (s, 1H), 6.96 (d, J=8.1 Hz, 1H), 4.82 (s, 2H), 4.62 (s, 2H), 3.42 (t, J=7.0 Hz, 2H), 2.94 (t, J=7.6 Hz, 2H), 2.83 (s, 1H), 1.71 (dp, J=14.7, 7.4 Hz, 4H), 1.48 (p, J=7.5 Hz, 2H), 0.88-0.60 (m, 2H), 0.56 (s, 2H). LCMS (ESI, +ve mode) expected m/z for [C₃₂H₃₆N₅O₅⁺] [M+H] 570.3, found 570.3.

### Example 7

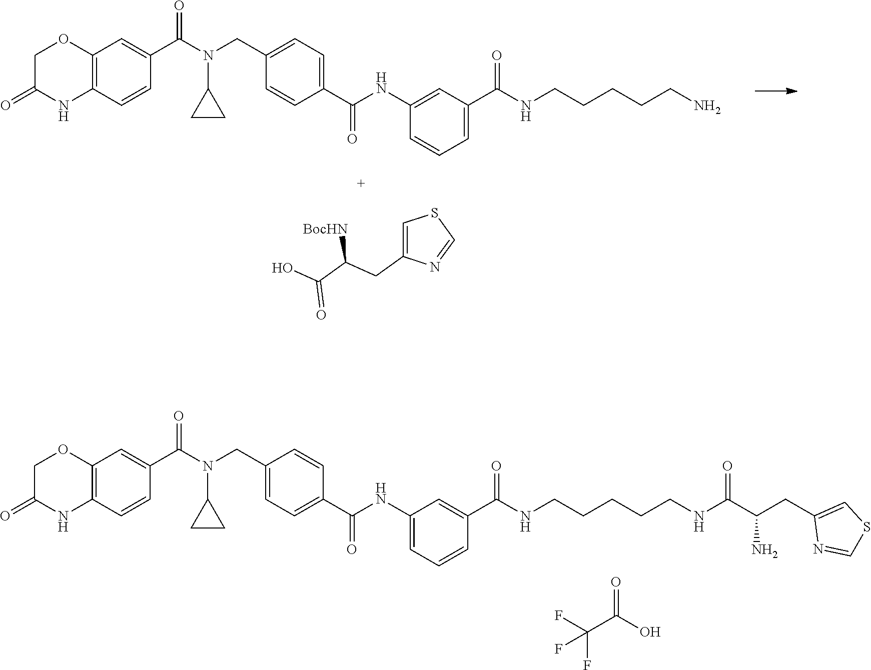 [SMILES: NCCCCCNC(=O)c1cccc(NC(=O)c2ccc(CN(C(=O)c3ccc4c(c3)OCC(=O)N4)C3CC3)cc2)c1 ; CC(C)(C)OC(=O)N[C@@H](Cc1cscn1)C(=O)O ; N[C@@H](Cc1cscn1)C(=O)NCCCCCNC(=O)c1cccc(NC(=O)c2ccc(CN(C(=O)c3ccc4c(c3)OCC(=O)N4)C3CC3)cc2)c1 ; O=C(O)C(F)(F)F]

Prepared according to General Procedure B/Workup A using Boc-3-(4-Thiazolyl)-L-alanine (10.43 mg, 1.1 Eq, 38.31 μmol), TBTU (14.54 mg, 1.3 Eq, 45.27 μmol), DMF (1 mL), N-(4-((3-((5-aminopentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (23.81 mg, 1 Eq, 34.83 μmol) and DIPEA (14.85 mg, 20.0 μL, 3.3 Eq, 114.9 μmol). Following N-Boc deprotection, the product was purified by reverse phase chromatography (10-100% methanol in water+0.1% TFA) to give (S)—N-(4-((3-((5-(2-amino-3-(thiazol-4-yl)propanamido)pentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (22.11 mg, 26.39 μmol, 75.77%) as a white solid. ¹H NMR (400 MHz, cd₃od) δ 8.99 (d, J=1.9 Hz, 1H), 8.20 (s, 1H), 7.96 (d, J=8.2 Hz, 2H), 7.85-7.70 (m, 1H), 7.58 (d, J=7.8 Hz, 1H), 7.50 (d, J=7.3 Hz, 2H), 7.45 (t, J=7.9 Hz, 1H), 7.38 (d, J=1.9 Hz, 1H), 7.26-7.10 (m, 2H), 6.96 (d, J=8.1 Hz, 1H), 4.83 (s, 2H), 4.62 (s, 2H), 4.19 (dd, J=7.7, 5.8 Hz, 1H), 3.42-3.36 (m, 2H), 3.36-3.32 (m, 1H), 3.30-3.22 (m, 2H), 3.16 (dt, J=13.4, 6.7 Hz, 1H), 2.84 (s, 1H), 1.64 (p, J=7.0 Hz, 2H), 1.53 (dt, J=13.9, 6.4 Hz, 2H), 1.36 (p, J=7.2 Hz, 2H), 0.86-0.62 (m, 2H), 0.62-0.38 (m, 2H). LCMS (ESI, +ve mode) expected m/z for [C₃₈H₄₂N₇O₆S⁺] [M+H] 724.3, found 724.3.

### Example 8

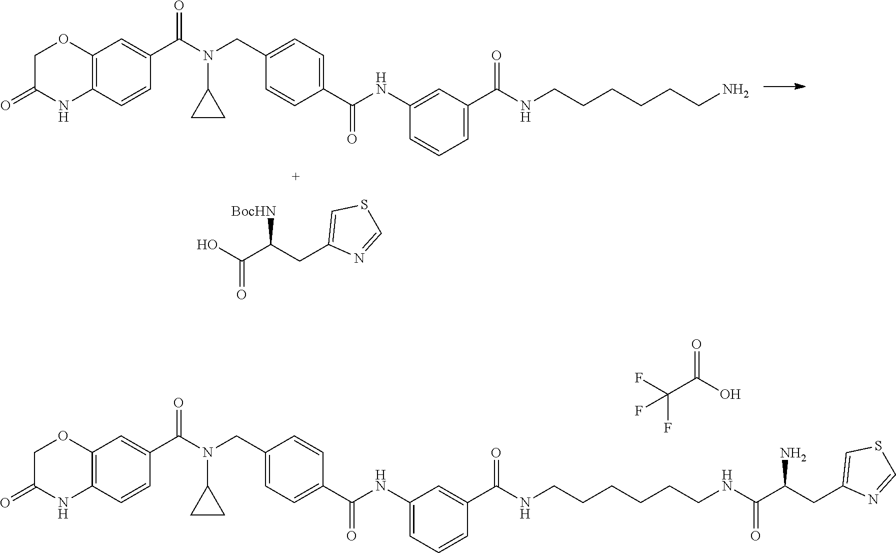 [SMILES: NCCCCCCNC(=O)c1cccc(NC(=O)c2ccc(CN(C(=O)c3ccc4c(c3)OCC(=O)N4)C3CC3)cc2)c1 ; CC(C)(C)OC(=O)N[C@@H](Cc1cscn1)C(=O)O ; N[C@@H](Cc1cscn1)C(=O)NCCCCCCNC(=O)c1cccc(NC(=O)c2ccc(CN(C(=O)c3ccc4c(c3)OCC(=O)N4)C3CC3)cc2)c1 ; O=C(O)C(F)(F)F]

Prepared according to General Procedure B/Workup A using Boc-3-(4-Thiazolyl)-L-alanine (8.6 mg, 1.1 Eq, 32 μmol), TBTU (12 mg, 1.3 Eq, 37 μmol), DMF (0.5 mL), N-(4-((3-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 29 μmol) and DIPEA (12 mg, 16 μL, 3.3 Eq, 95 μmol). Following N-Boc deprotection, the product was purified by reverse phase chromatography (10-100% methanol in water+0.1% TFA) to give (S)—N-(4-((3-((6-(2-amino-3-(thiazol-4-yl)propanamido)hexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5.74 mg, 6.74 μmol, 24%) as a white solid. ¹H NMR (400 MHz, cd₃od) δ 8.97 (d, J=1.9 Hz, 1H), 8.16 (s, 1H), 7.93 (d, J=8.2 Hz, 2H), 7.76 (d, J=8.0 Hz, 1H), 7.54 (d, J=7.9 Hz, 1H), 7.46 (d, J=7.5 Hz, 2H), 7.42 (t, J=7.9 Hz, 2H), 7.38 (d, J=1.8 Hz, 1H), 7.24-7.05 (m, 2H), 6.93 (d, J=8.0 Hz, 1H), 4.79 (s, 2H), 4.59 (s, 2H), 4.16 (dd, J=7.5, 5.9 Hz, 1H), 3.42-3.30 (m, 10H), 3.21 (dt, J=13.6, 6.9 Hz, 3H), 3.17-3.03 (m, 1H), 2.80 (s, 1H), 1.60 (p, J=7.1 Hz, 2H), 1.44 (p, J=7.0 Hz, 2H), 1.41-1.25 (m, 5H), 0.73-0.57 (m, 2H), 0.57-0.31 (m, 2H). LCMS (ESI, +ve mode) expected m/z for [C₃₉H₄₄N₇O₆S⁺] [M+H] 738.3, found 738.2.

### Example 9

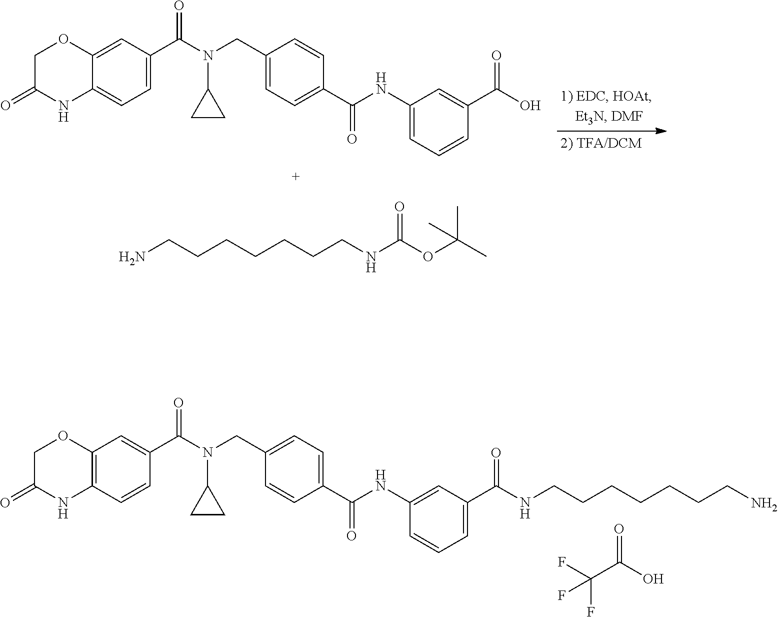

Prepared according to General Procedure A/Workup B using 3-(4-((N-cyclopropyl-3-oxo-3,4-dihydro-2H-benzo[b][1,4]oxazine-7-carboxamido)methyl)benzamido)benzoic acid (60.0 mg, 1 Eq, 124 μmol), EDC (35.5 mg, 1.5 Eq, 185 μmol), HOAt (25.2 mg, 1.5 Eq, 185 μmol), DMF (1 mL), tert-butyl (7-aminoheptyl)carbamate (42.7 mg, 1.5 Eq, 185 μmol) and triethylamine (50.0 mg, 69 μL, 4 Eq, 494 μmol). Following N-Boc deprotection, the product was purified by reverse phase chromatography (10-100% methanol in water+0.1% TFA) to give N-(4-((3-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (6.24 mg, 8.77 μmol, 7.09%) as a white solid. ¹H NMR (400 MHz, cd₃od) δ 8.19 (t, J=1.7 Hz, 1H), 7.96 (d, J=8.2 Hz, 2H), 7.81 (d, J=8.0 Hz, 1H), 7.57 (d, J=7.8 Hz, 1H), 7.49 (s, 1H), 7.48-7.38 (m, 2H), 7.25-7.09 (m, 2H), 6.96 (d, J=8.1 Hz, 1H), 4.82 (s, 2H), 4.61 (s, 2H), 3.39 (t, J=7.1 Hz, 2H), 2.91 (t, J=7.6 Hz, 2H), 2.82 (s, 1H), 1.75-1.53 (m, 4H), 1.42 (s, 6H), 0.64 (s, 2H), 0.55 (s, 2H). LCMS (ESI, +ve mode) expected m/z for [C₃₄H₄₀N₅O₅⁺] [M+H] 598.3, found 598.3.

### Example 10

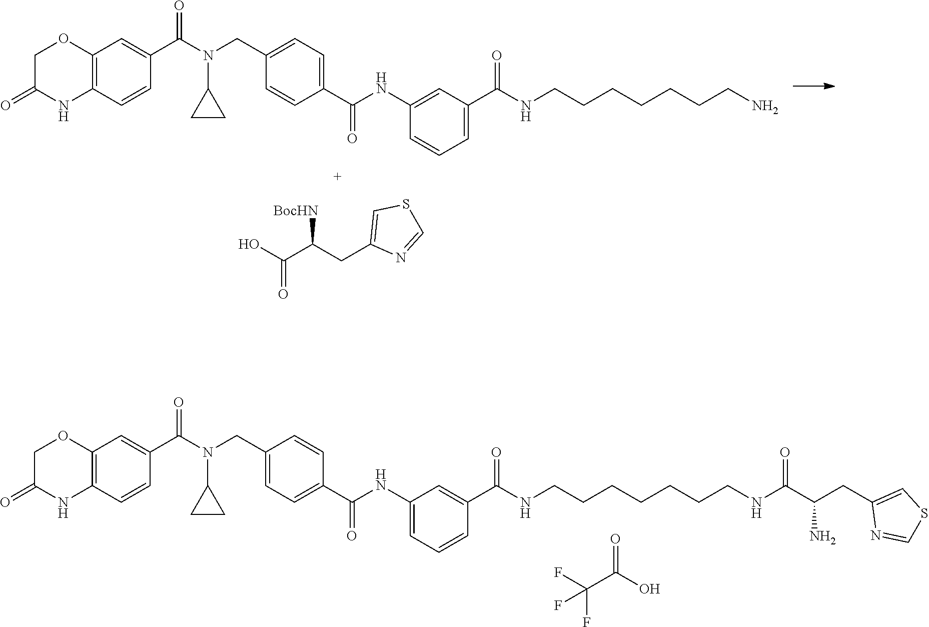

Prepared according to General Procedure B/Workup A using Boc-3-(4-Thiazolyl)-L-alanine (6.14 mg, 1.1 Eq, 22.6 μmol), TBTU (8.56 mg, 1.3 Eq, 26.7 μmol), DMF (0.5 mL), N-(4-((3-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (14.6 mg, 1 Eq, 20.5 μmol) and DIPEA (8.75 mg, 11.8 μL, 3.3 Eq, 67.7 μmol). Following N-Boc deprotection, the product was purified by reverse phase chromatography (10-100% methanol in water+0.1% TFA) to give (S)—N-(4-((3-((7-(2-amino-3-(thiazol-4-yl)propanamido)heptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (3.11 mg, 3.59 μmol, 17.5%) as a white solid. ¹H NMR (400 MHz, cd₃od) δ 9.00 (d, J=1.9 Hz, 1H), 8.18 (s, 1H), 7.97 (d, J=8.2 Hz, 2H), 7.80 (d, J=8.0 Hz, 1H), 7.57 (d, J=7.9 Hz, 1H), 7.50 (d, J=7.6 Hz, 2H), 7.46 (t, J=7.9 Hz, 1H), 7.40 (d, J=1.9 Hz, 1H), 7.26-7.11 (m, 2H), 6.97 (d, J=8.1 Hz, 1H), 4.84 (s, 2H), 4.63 (s, 2H), 4.19 (dd, J=7.5, 5.9 Hz, 1H), 3.39 (t, J=7.2 Hz, 2H), 3.36-3.32 (m, 1H), 3.30-3.19 (m, 2H), 3.14 (dt, J=13.6, 6.9 Hz, 1H), 2.84 (s, 1H), 1.64 (p, J=7.0 Hz, 2H), 1.54-1.43 (m, 2H), 1.43-1.34 (m, 4H), 1.34-1.28 (m, 2H), 0.76-0.62 (m, 2H), 0.62-0.40 (m, 2H). LCMS (ESI, +ve mode) expected m/z for [C₄₀H₄₆N₇O₆S⁺] [M+H] 752.3, found 752.2.

### Example 11

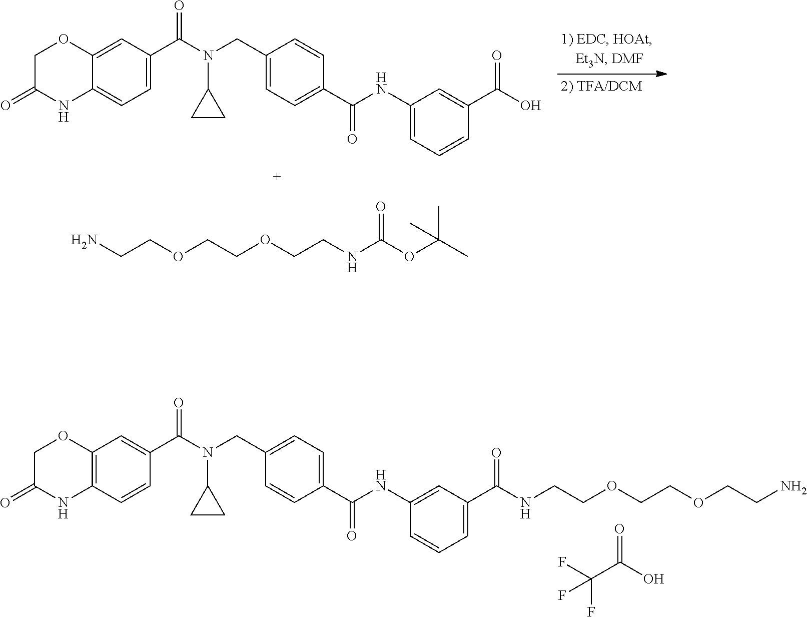

Prepared according to General Procedure A/Workup B using 3-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 154 μmol), HOAt (21.0 mg, 1.5 Eq, 154 μmol), DMF (1 mL), tert-butyl (2-(2-(2-aminoethoxy)ethoxy)ethyl)carbamate (38.4 mg, 1.5 Eq, 154 μmol) and triethylamine (41.7 mg, 57 μL, 4 Eq, 412 μmol). Following N-Boc deprotection, the product was purified by reverse phase chromatography (10-100% methanol in water+0.1% TFA) to give N-(4-((3-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (7.57 mg, 10.4 μmol, 10.1%) as a white solid. ¹H NMR (400 MHz, cd₃od) δ 8.25 (t, J=1.7 Hz, 1H), 7.97 (d, J=8.2 Hz, 2H), 7.83-7.70 (m, 1H), 7.60 (d, J=8.2 Hz, 1H), 7.51 (s, 1H), 7.50-7.38 (m, 2H), 7.27-7.12 (m, 2H), 6.96 (d, J=8.1 Hz, 1H), 4.83 (s, 2H), 4.62 (s, 2H), 3.79-3.65 (m, 8H), 3.61 (t, J=5.5 Hz, 2H), 3.18-2.97 (m, 2H), 2.84 (s, 1H), 0.86-0.62 (m, 2H), 0.57 (s, 2H). LCMS (ESI, +ve mode) expected m/z for [C₃₃H₃₈N₅O₇⁺] [M+H] 616.3, found 616.2.

### Example 12

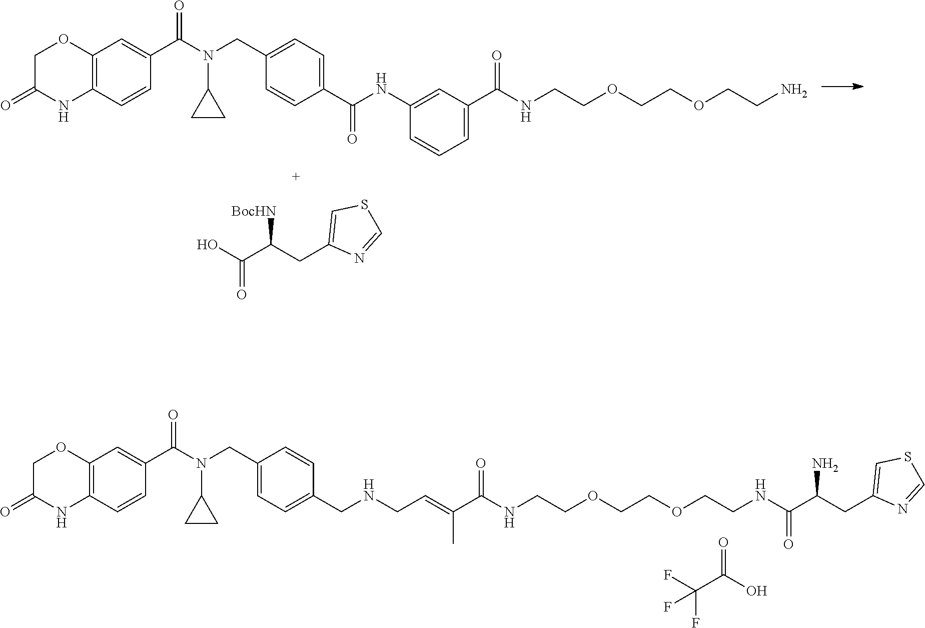

Prepared according to General Procedure B/Workup A using Boc-3-(4-Thiazolyl)-L-alanine (15.6 mg, 1.1 Eq, 57.4 μmol), TBTU (21.8 mg, 1.3 Eq, 67.9 μmol), DMF (1 mL), N-(4-((3-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (38.1 mg, 1 Eq, 52.2 μmol) and DIPEA (22.3 mg, 30.0 μL, 3.3 Eq, 172 μmol). Following N-Boc deprotection, the product was purified by reverse phase chromatography (10-100% methanol in water+0.10% TFA) to give (S)—N-(4-((3-((2-(2-(2-(2-amino-3-(thiazol-4-yl)propanamido)ethoxy)ethoxy) ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (20.04 mg, 22.67 μmol, 43.4%) as a white solid. ¹H NMR (400 MHz, cd₃od) δ 8.98 (d, J=1.9 Hz, 1H), 8.21 (t, J=1.7 Hz, 1H), 7.96 (d, J=8.2 Hz, 2H), 7.79 (d, J=8.0 Hz, 1H), 7.58 (d, J=7.8 Hz, 1H), 7.50 (d, J=7.9 Hz, 2H), 7.46 (t, J=7.9 Hz, 1H), 7.41 (d, J=1.9 Hz, 1H), 7.26-7.07 (m, 2H), 6.96 (d, J=8.1 Hz, 1H), 4.83 (s, 2H), 4.62 (s, 2H), 4.22 (dd, J=7.6, 5.5 Hz, 1H), 3.77-3.65 (m, 4H), 3.65-3.56 (m, 4H), 3.56-3.46 (m, 2H), 3.46-3.34 (m, 2H), 3.34-3.32 (m, 1H), 3.30-3.22 (m, 1H), 2.84 (s, 1H), 0.86-0.61 (m, 2H), 0.62-0.40 (m, 2H). LCMS (ESI, +ve mode) expected m/z for [C₃₉H₄₄N₇O₈S⁺] [M+H] 770.3, found 770.2.

### Example 13

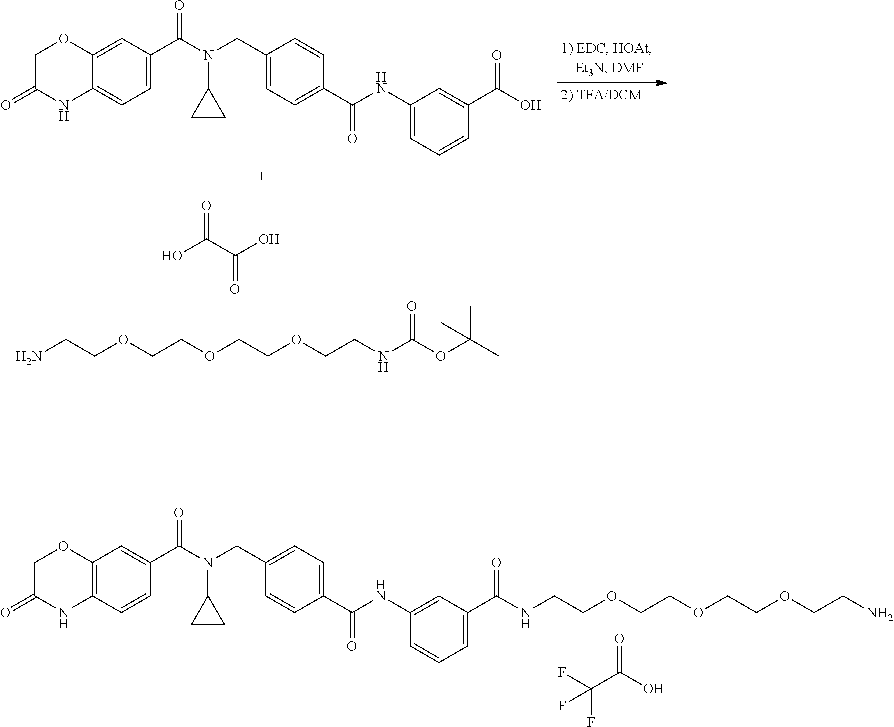

Prepared according to General Procedure A/Workup B using 3-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 155 μmol), HOAt (21.0 mg, 1.5 Eq, 155 μmol), DMF (1 mL), tert-butyl (2-(2-(2-(2-aminoethoxy)ethoxy)ethoxy)ethyl)carbamate oxalate (39.4 mg, 1 Eq, 103 μmol) and triethylamine (41.7 mg, 57 μL, 4 Eq, 412 μmol). Following N-Boc deprotection, the product was purified by reverse phase chromatography (10-100% methanol in water+0.10% TFA) to give N-(4-((3-((2-(2-(2-(2-aminoethoxy)ethoxy)ethoxy)ethyl)carbamoyl)phenyl) carbamoyl)benzyl)-N-cyclopropyl-3-oxo-3,4-dihydro-2H-benzo[b][1,4]oxazine-7-carboxamide 2,2,2-trifluoroacetate (8.99 mg, 11.6 μmol, 11.3%) as a white solid. ¹H NMR (400 MHz, cd₃od) δ 8.22 (t, J=1.7 Hz, 1H), 7.97 (d, J=8.2 Hz, 2H), 7.87-7.69 (m, 1H), 7.60 (d, J=7.8 Hz, 1H), 7.52 (s, 1H), 7.51-7.40 (m, 2H), 7.21 (d, J=8.1 Hz, 1H), 7.18 (s, 1H), 6.97 (d, J=8.1 Hz, 1H), 4.84 (s, 2H), 4.63 (s, 2H), 3.78-3.48 (m, 14H), 3.15-3.03 (m, 2H), 2.85 (s, 1H), 0.84-0.61 (m, 2H), 0.61-0.38 (m, 2H). LCMS (ESI, +ve mode) expected m/z for [C₃₅H₄₂N₅O₈⁺] [M+H] 660.3, found 660.3.

### Example 14

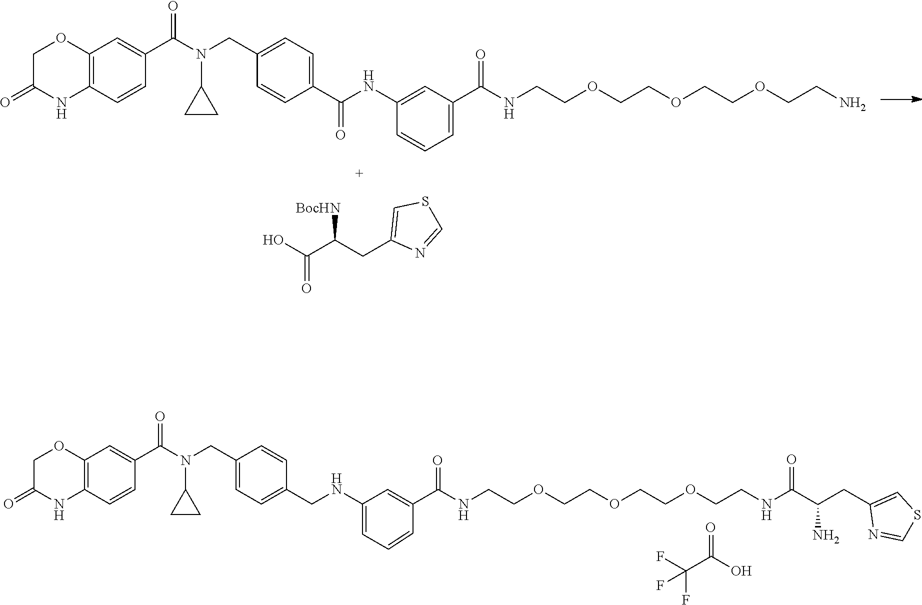

Prepared according to General Procedure B/Workup A using Boc-3-(4-Thiazolyl)-L-alanine (19.0 mg, 1.1 Eq, 69.7 μmol), TBTU (26.4 mg, 1.3 Eq, 82.3 μmol), DMF (1 mL), N-(4-((3-((2-(2-(2-(2-aminoethoxy)ethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (49.0 mg, 1 Eq, 63.3 μmol) and DIPEA (27.0 mg, 36.4 μL, 3.3 Eq, 209 μmol). Following N-Boc deprotection, the product was purified by reverse phase chromatography (10-100% methanol in water+0.1% TFA) to give (S)—N-(4-((3-((14-amino-13-oxo-15-(thiazol-4-yl)-3,6,9-trioxa-12-azapentadecyl) carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (25.79 mg, 27.79 μmol, 43.9%) as a white solid. ¹H NMR (400 MHz, cd₃od) δ 8.99 (d, J=1.9 Hz, 1H), 8.19 (t, J=1.7 Hz, 1H), 7.96 (d, J=8.2 Hz, 2H), 7.88-7.69 (m, 1H), 7.59 (d, J=7.9 Hz, 1H), 7.57-7.44 (m, 3H), 7.42 (d, J 1.9 Hz, 1H), 7.27-7.06 (m, 2H), 6.96 (d, J=8.1 Hz, 1H), 4.83 (s, 2H), 4.62 (s, 2H), 4.23 (dd, J=7.6, 5.5 Hz, 1H), 3.75-3.61 (m, 8H), 3.61-3.51 (m, 4H), 3.51-3.34 (m, 4H), 3.31-3.16 (m, 2H), 2.84 (s, 1H), 0.92-0.61 (m, 2H), 0.61-0.40 (m, 2H). LCMS (ESI, +ve mode) expected m/z for [C₄₁H₄₈N₇O₉S⁺] [M+H] 814.3, found 814.3.

### Example 15

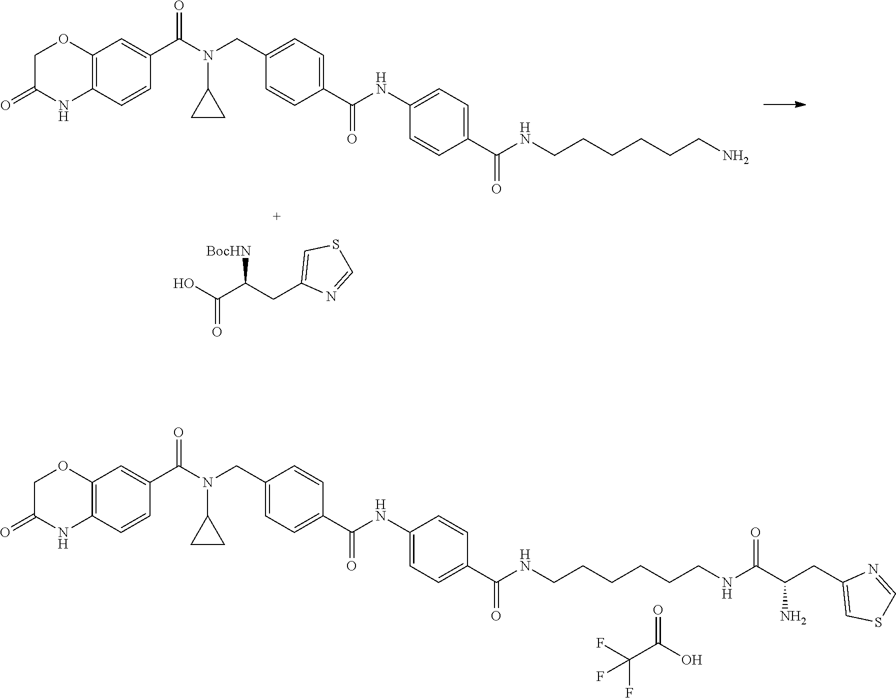

Prepared according to General Procedure B/Workup A using Boc-3-(4-Thiazolyl)-L-alanine (8.59 mg, 1.1 Eq, 31.5 μmol), TBTU (12.0 mg, 1.3 Eq, 37.3 μmol), DMF (1 mL), N-(4-((4-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (20.0 mg, 1 Eq, 28.7 μmol) and DIPEA (12.2 mg, 16.5 μL, 3.3 Eq, 94.6 μmol). Following N-Boc deprotection, the product was purified by reverse phase chromatography (10-100% methanol in water+0.1% TFA) to give o give (S)—N-(4-((4-((6-(2-amino-3-(thiazol-4-yl)propanamido)hexyl)carbamoyl)phenyl) carbamoyl)benzyl)-N-cyclopropyl-3-oxo-3,4-dihydro-2H-benzo[b][1,4]oxazine-7-carboxamide 2,2,2-trifluoroacetate (10.49 mg, 12.31 μmol, 43.0%) as a white solid. ¹H NMR (400 MHz, cd₃od) δ 9.02 (d, J=1.9 Hz, 1H), 7.96 (d, J=8.2 Hz, 2H), 7.84 (s, 4H), 7.50 (d, J=8.0 Hz, 2H), 7.42 (s, 1H), 7.20 (d, J=8.1 Hz, 1H), 7.18 (s, 1H), 6.96 (d, J=8.0 Hz, 1H), 4.83 (s, 2H), 4.62 (s, 2H), 4.30-4.09 (m, 1H), 3.48-3.32 (m, 4H), 3.27-3.20 (m, 1H), 3.20-3.09 (m, 1H), 2.83 (s, 1H), 1.71-1.55 (m, 2H), 1.55-1.44 (m, 2H), 1.44-1.19 (m, 4H), 0.82-0.61 (m, 2H), 0.61-0.42 (m, 2H). LCMS (ESI, +ve mode) expected m/z for [C₃₉H₄₄N₇O₆S⁺] [M+H] 738.3, found 738.2.

### Example 16

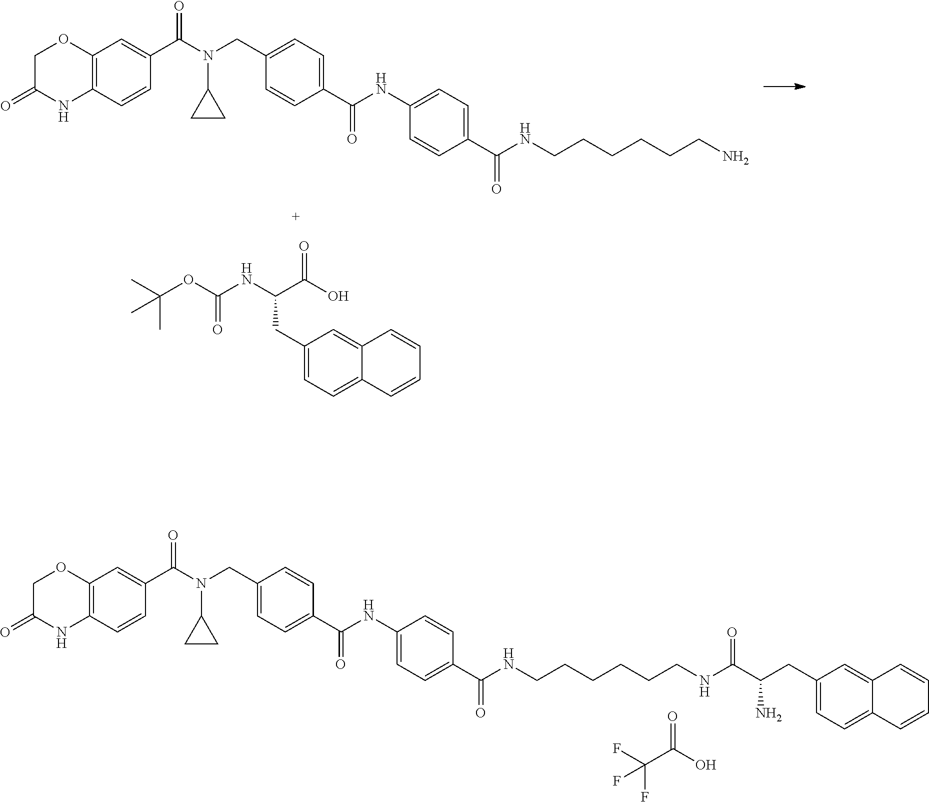

Prepared according to General Procedure B/Workup A using Boc-3-(4-Thiazolyl)-L-alanine (7.5 mg, 1.1 Eq, 24 μmol), TBTU (9.0 mg, 1.3 Eq, 28 μmol), DMF (1 mL), N-(4-((4-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (15 mg, 1 Eq, 21 μmol) and DIPEA (9.2 mg, 12 μL, 3.3 Eq, 71 μmol). The N-Boc protected product was purified by normal phase chromatography over silica gel (0-10% MeOH in DCM). Following N-Boc deprotection, volatiles were removed in vacuo to give (S)—N-(4-((4-((6-(2-amino-3-(naphthalen-2-yl)propanamido)hexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (6.26 mg, 6.99 μmol, 33%) as a white solid. ¹H NMR (400 MHz, cd₃od) δ 7.95 (d, J=8.3 Hz, 2H), 7.92-7.77 (m, 7H), 7.72 (s, 1H), 7.47 (ddt, J=9.6, 6.9, 5.2 Hz, 4H), 7.39 (dd, J=8.4, 1.6 Hz, 1H), 7.25-7.11 (m, 2H), 6.96 (d, J=8.1 Hz, 1H), 4.83 (s, 3H), 4.62 (s, 2H), 4.08 (t, J=7.6 Hz, 1H), 3.31-3.13 (m, 12H), 3.00 (dt, J=13.4, 6.3 Hz, 1H), 2.82 (s, 1H), 1.45 (p, J=7.1 Hz, 2H), 1.38-1.21 (m, 3H), 1.23-1.12 (m, 2H), 1.13-0.91 (m, 2H), 0.76-0.60 (m, 2H), 0.60-0.39 (m, 2H). LCMS (ESI, +ve mode) expected m/z for [C₄₆H₄₉N₆O₆⁺] [M+H] 781.4, found 781.3.

### Example 17

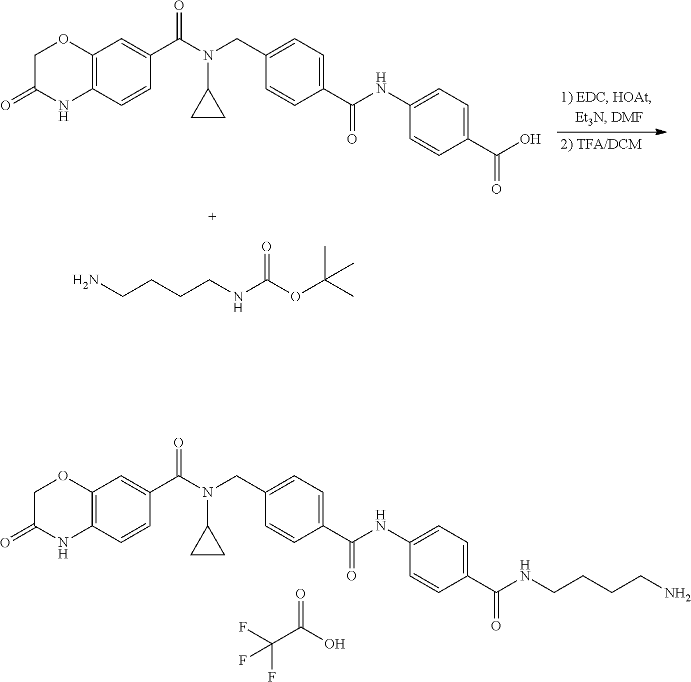 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCCCN ; NCCCCNC(=O)c1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup B using 4-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 155 μmol), HOAt (21.0 mg, 1.5 Eq, 155 μmol), DMF (1 mL), tert-butyl (4-aminobutyl)carbamate (29.1 mg, 1.5 Eq, 155 μmol) and triethylamine (41.7 mg, 57 μL, 4 Eq, 412 μmol). Following N-Boc deprotection, the product was purified by reverse phase chromatography (10-100% methanol in water+0.1% TFA) to give N-(4-((4-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (8.6 mg, 13 μmol, 12%) as a white solid. ¹H NMR (400 MHz, cd₃od) δ 7.96 (d, J=8.3 Hz, 2H), 7.85 (s, 4H), 7.50 (d, J=7.4 Hz, 2H), 7.30-7.10 (m, 2H), 6.96 (d, J=8.1 Hz, 1H), 4.83 (s, 2H), 4.62 (s, 2H), 3.44 (t, J=6.0 Hz, 2H), 2.98 (d, J=7.0 Hz, 2H), 2.83 (s, 1H), 1.83-1.62 (m, 4H), 0.65 (s, 2H), 0.57 (s, 2H). LCMS (ESI, +ve mode) expected m/z for [C₃₁H₃₄N₅O₅⁺] [M+H] 556.3, found 556.2.

### Example 18

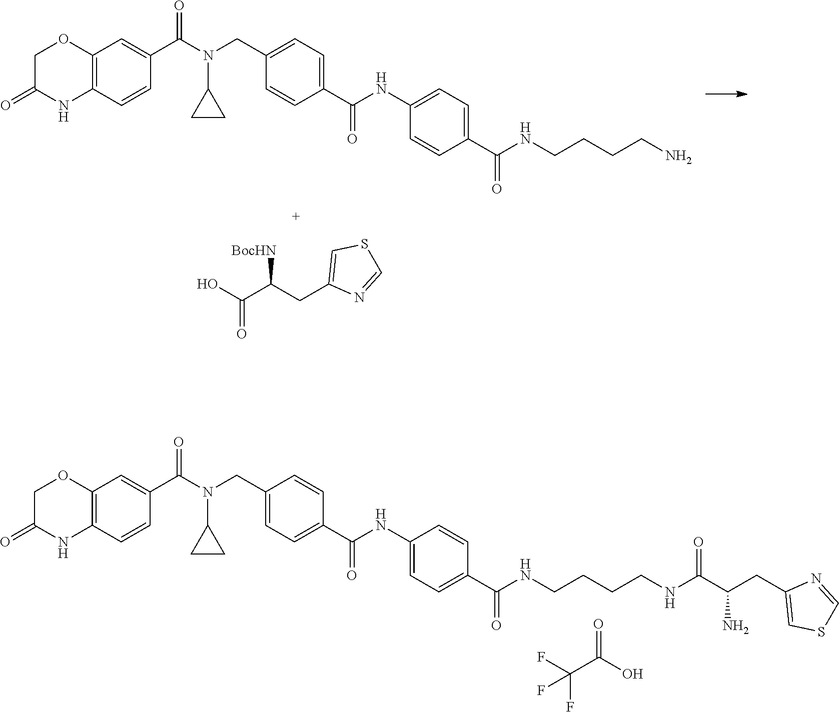

Prepared according to General Procedure B/Workup A using Boc-3-(4-Thiazolyl)-L-alanine (3.83 mg, 1.1 Eq, 14.1 μmol), TBTU (5.34 mg, 1.3 Eq, 16.6 μmol), DMF (0.5 mL), N-(4-((4-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (8.57 mg, 1 Eq, 12.8 μmol) and DIPEA (5.46 mg, 7.36 μL, 3.3 Eq, 42.2 μmol). The N-Boc protected product was purified by normal phase chromatography over silica gel (0-10% MeOH in DCM). Following N-Boc deprotection, volatiles were removed in vacuo to give (S)—N-(4-((4-((4-(2-amino-3-(thiazol-4-yl)propanamido)butyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (3.40 mg, 4.13 μmol, 32.2%) as a white solid. ¹H NMR (400 MHz, cd₃od) δ 9.01 (d, J=1.9 Hz, 1H), 7.96 (d, J=8.2 Hz, 2H), 7.91-7.75 (m, 4H), 7.51 (d, J=7.7 Hz, 2H), 7.42 (d, J=1.9 Hz, 1H), 7.26-7.11 (m, 2H), 6.96 (d, J=8.0 Hz, 1H), 4.84 (s, 2H), 4.63 (s, 2H), 4.19 (dd, J=7.5, 5.8 Hz, 1H), 3.44-3.35 (m, 4H), 3.29-3.24 (m, 1H), 3.24-3.11 (m, 1H), 2.84 (s, 1H), 1.72-1.44 (m, 4H), 0.75-0.61 (m, 2H), 0.61-0.36 (m, 2H). LCMS (ESI, +ve mode) expected m/z for [C₃₇H₄₀N₇O₆S⁺] [M+H] 710.3, found 710.2.

### Example 19

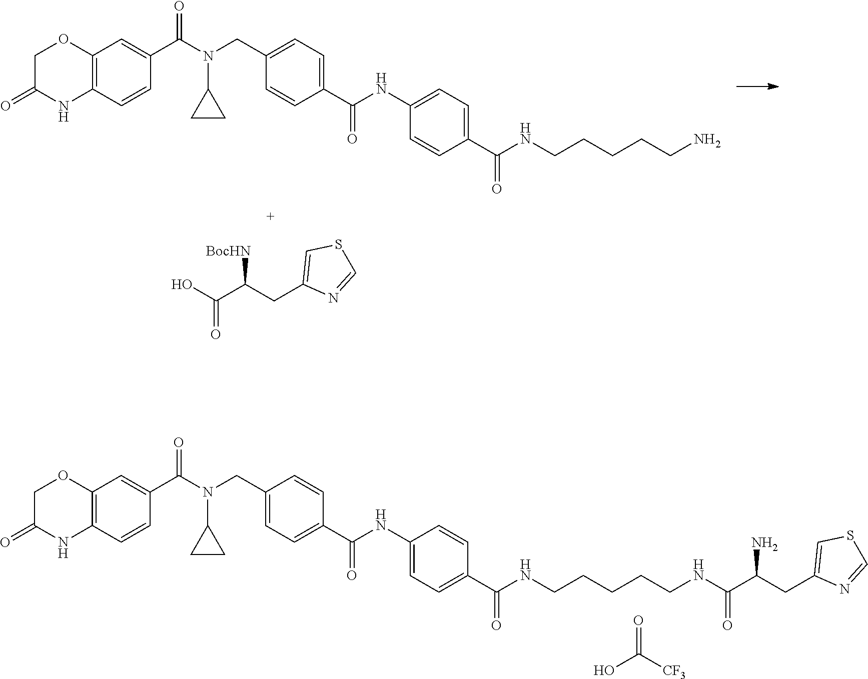

Prepared according to General Procedure B/Workup A using Boc-3-(4-Thiazolyl)-L-alanine (4.8 mg, 1.1 Eq, 18 μmol), TBTU (6.7 mg, 1.3 Eq, 21 μmol), DMF (0.5 mL), N-(4-((4-((5-aminopentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (11 mg, 1 Eq, 16 μmol) and DIPEA (6.9 mg, 9.2 μL, 3.3 Eq, 53 μmol). The N-Boc protected product was purified by normal phase chromatography over silica gel (0-10% MeOH in DCM). Following N-Boc deprotection, volatiles were removed in vacuo to give (S)—N-(4-((4-((5-(2-amino-3-(thiazol-4-yl)propanamido)pentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (9.01 mg, 10.8 μmol, 67%) as a white solid. ¹H NMR (400 MHz, cd₃od) δ 9.01 (d, J=1.9 Hz, 1H), 7.96 (d, J=8.2 Hz, 2H), 7.91-7.75 (m, 4H), 7.50 (d, J=7.4 Hz, 2H), 7.39 (d, J=1.8 Hz, 1H), 7.28-7.08 (m, 2H), 6.96 (d, J=8.0 Hz, 1H), 4.83 (s, 2H), 4.62 (s, 2H), 4.18 (dd, J=7.7, 5.8 Hz, 1H), 3.46-3.32 (m, 4H), 3.28-3.23 (m, 1H), 3.21-3.10 (m, 1H), 2.83 (s, 1H), 1.63 (p, J=7.3 Hz, 2H), 1.53 (dt, J=14.7, 6.9 Hz, 2H), 1.44-1.25 (m, 2H), 0.79-0.61 (m, 2H), 0.61-0.42 (m, 2H). LCMS (ESI, +ve mode) expected m/z for [C₃₈H₄₂N₇O₆S⁺] [M+H] 724.3, found 724.2.

### Example 20

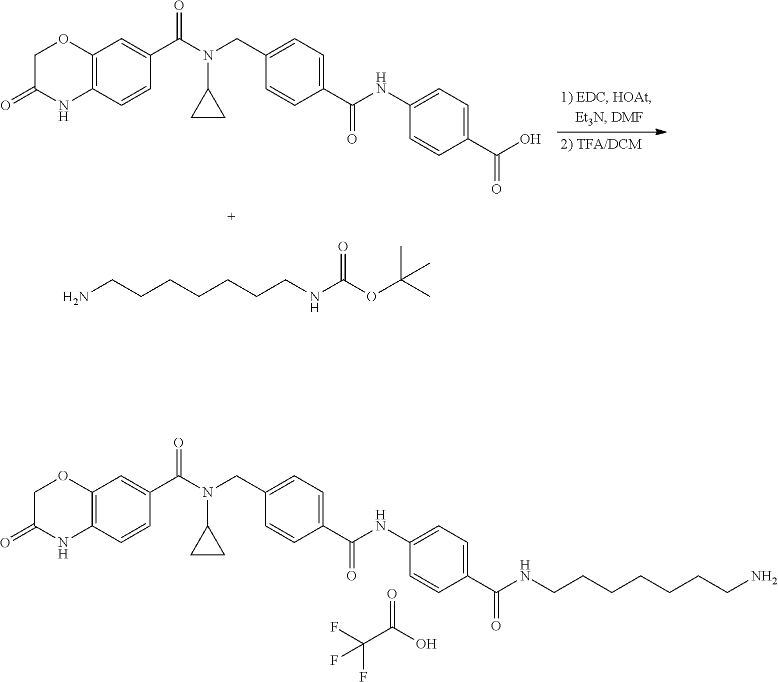

Prepared according to General Procedure A/Workup B using 4-(4-((N-cyclopropyl-3-oxo-3,4-dihydro-2H-benzo[b][1,4]oxazine-7-carboxamido)methyl)benzamido)benzoic acid (100 mg, 1 Eq, 206 μmol), EDC (59.2 mg, 1.5 Eq, 309 μmol), HOAt (42.1 mg, 1.5 Eq, 309 μmol), DMF (2 mL), tert-butyl (7-aminoheptyl)carbamate (71.2 mg, 1.5 Eq, 309 μmol) and triethylamine (83.4 mg, 0.11 mL, 4 Eq, 824 μmol). Following N-Boc deprotection, the product was purified by reverse phase chromatography (10-100% methanol in water+0.1% TFA) to give N-(4-((4-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (18.94 mg, 26.61 μmol, 12.9%) as a white solid. ¹H NMR (400 MHz, cd₃od) δ 7.95 (d, J=8.2 Hz, 2H), 7.84 (s, 4H), 7.49 (d, J=7.5 Hz, 2H), 7.24-7.09 (m, 2H), 6.96 (d, J=8.0 Hz, 1H), 4.82 (s, 2H), 4.62 (s, 2H), 3.38 (t, J=7.1 Hz, 2H), 2.92 (t, J=7.6 Hz, 2H), 2.82 (s, 1H), 1.65 (d, J=6.3 Hz, 4H), 1.42 (s, 6H), 0.89-0.60 (m, 2H), 0.60-0.41 (m, 2H). LCMS (ESI, +ve mode) expected m/z for [C₃₄H₄₀N₅O₅⁺] [M+H] 598.3, found 598.3.

### Example 21

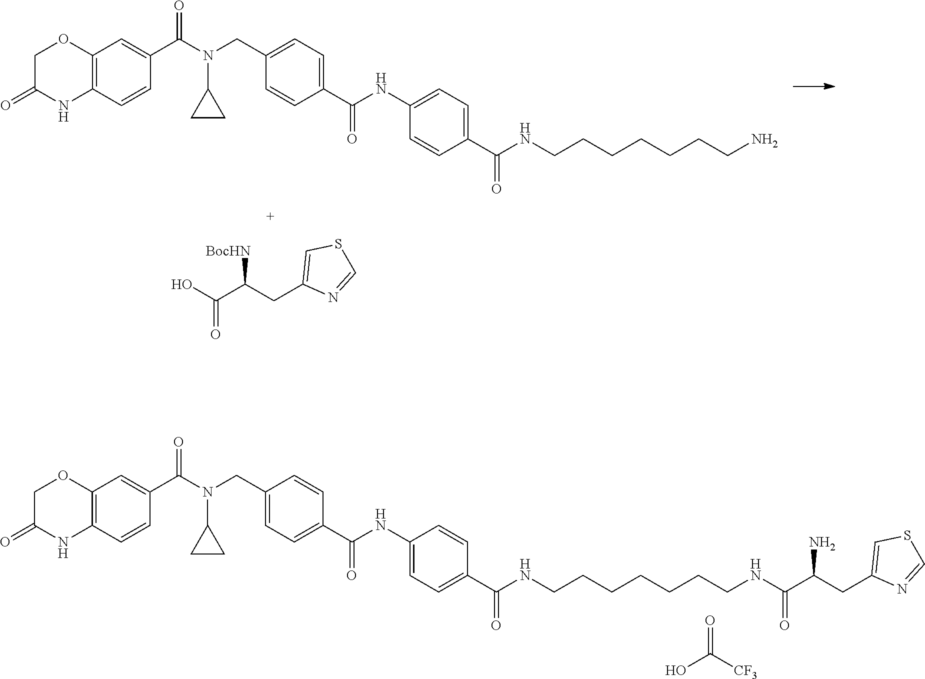

Prepared according to General Procedure B/Workup A using Boc-3-(4-Thiazolyl)-L-alanine (5.13 mg, 1.1 Eq, 18.9 μmol), TBTU (7.15 mg, 1.3 Eq, 22.3 μmol), DMF (1.5 mL), N-(4-((4-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (12.2 mg, 1 Eq, 17.1 μmol) and DIPEA (7.31 mg, 9.85 μL, 3.3 Eq, 56.6 μmol). Following N-Boc deprotection, the product was purified by reverse phase chromatography (10-100% methanol in water+0.1% TFA) to give (S)—N-(4-((4-((7-(2-amino-3-(thiazol-4-yl)propanamido)heptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (9.49 mg, 11.0 μmol, 63.9%) as a white solid. ¹H NMR (400 MHz, cd₃od) δ 9.01 (d, J=1.9 Hz, 1H), 7.96 (d, J=8.3 Hz, 2H), 7.89-7.76 (m, 4H), 7.50 (d, J=7.7 Hz, 2H), 7.41 (d, J=1.9 Hz, 1H), 7.27-7.11 (m, 2H), 6.96 (d, J=8.0 Hz, 1H), 4.83 (s, 2H), 4.62 (s, 2H), 4.19 (dd, J=7.6, 5.8 Hz, 1H), 3.44-3.33 (m, 3H), 3.30-3.18 (m, 2H), 3.13 (dt, J=13.6, 6.9 Hz, 1H), 2.83 (s, 1H), 1.70-1.55 (m, 2H), 1.55-1.42 (m, 2H), 1.42-1.34 (m, 4H), 1.34-1.26 (m, 2H), 0.82-0.61 (m, 2H), 0.61-0.41 (m, 2H). LCMS (ESI, +ve mode) expected m/z for [C₄₀H₄₆N₇O₆S⁺] [M+H] 752.3, found 752.3.

### Example 22

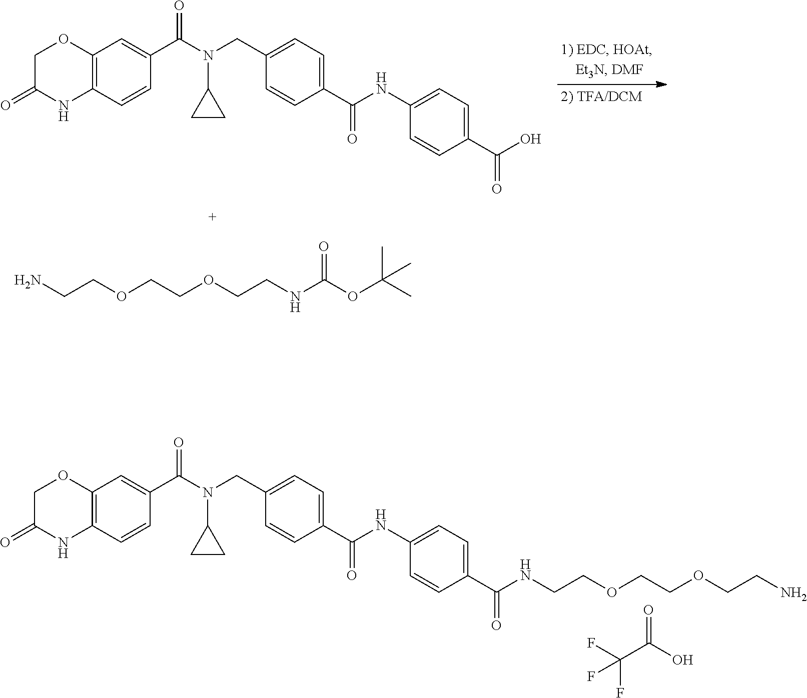

Prepared according to General Procedure A/Workup B using 4-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 155 μmol), HOAt (21.0 mg, 1.5 Eq, 155 μmol), DMF (1 mL), tert-butyl (2-(2-(2-aminoethoxy)ethoxy)ethyl)carbamate (38.4 mg, 1.5 Eq, 155 μmol) and triethylamine (41.7 mg, 57 μL, 4 Eq, 412 μmol). Following N-Boc deprotection, the product was purified by reverse phase chromatography (10-100% methanol in water+0.1% TFA) to give N-(4-((4-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (9.9 mg, 14 μmol, 13%) as a white solid. ¹H NMR (400 MHz, cd₃od) δ 7.96 (d, J=8.2 Hz, 2H), 7.85 (s, 4H), 7.50 (d, J=7.6 Hz, 2H), 7.28-7.10 (m, 2H), 6.96 (d, J=8.0 Hz, 1H), 4.83 (s, 2H), 4.62 (s, 2H), 3.74-3.65 (m, 8H), 3.60 (t, J=5.6 Hz, 2H), 3.13-3.03 (m, 2H), 2.83 (s, 1H), 0.92-0.61 (m, 2H), 0.61-0.47 (m, 2H). LCMS (ESI, +ve mode) expected m/z for [C₃₃H₃₈N₅O₇⁺] [M+H] 616.3, found 616.2.

### Example 23

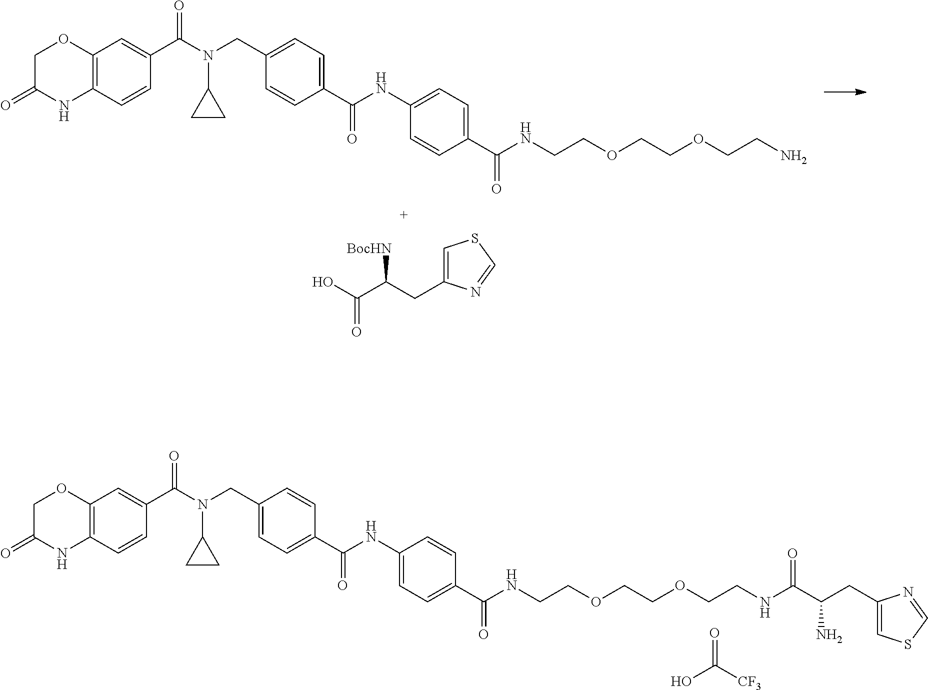

Prepared according to General Procedure B/Workup A using Boc-3-(4-Thiazolyl)-L-alanine (3.35 mg, 1.1 Eq, 12.3 μmol), TBTU (4.68 mg, 1.3 Eq, 14.6 μmol), DMF (0.5 mL), N-(4-((4-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (8.17 mg, 1 Eq, 11.2 μmol) and DIPEA (4.78 mg, 6.44 μL, 3.3 Eq, 37.0 μmol). The N-Boc protected product was purified by normal phase chromatography over silica gel (0-10% MeOH in DCM). Following N-Boc deprotection, volatiles were removed in vacuo to give (S)—N-(4-((4-((2-(2-(2-(2-amino-3-(thiazol-4-yl)propanamido)ethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (4.18 mg, 4.73 μmol, 42.2%) as a white solid. ¹H NMR (400 MHz, cd₃od) δ 8.99 (d, J=1.9 Hz, 1H), 7.96 (d, J=8.2 Hz, 2H), 7.90-7.72 (m, 4H), 7.51 (d, J=7.7 Hz, 2H), 7.42 (d, J=1.9 Hz, 1H), 7.26-7.11 (m, 2H), 6.96 (d, J=8.1 Hz, 1H), 4.84 (s, 2H), 4.63 (s, 2H), 4.22 (dd, J=7.6, 5.5 Hz, 1H), 3.76-3.64 (m, 4H), 3.64-3.56 (m, 4H), 3.56-3.45 (m, 2H), 3.45-3.35 (m, 2H), 3.34-3.32 (m, 1H), 3.30-3.23 (m, 1H), 2.84 (s, 1H), 0.76-0.61 (m, 2H), 0.60-0.34 (m, 2H). LCMS (ESI, +ve mode) expected m/z for [C₃₉H₄₄N₇O₈S⁺] [M+H] 770.3, found 770.2.

### Example 24

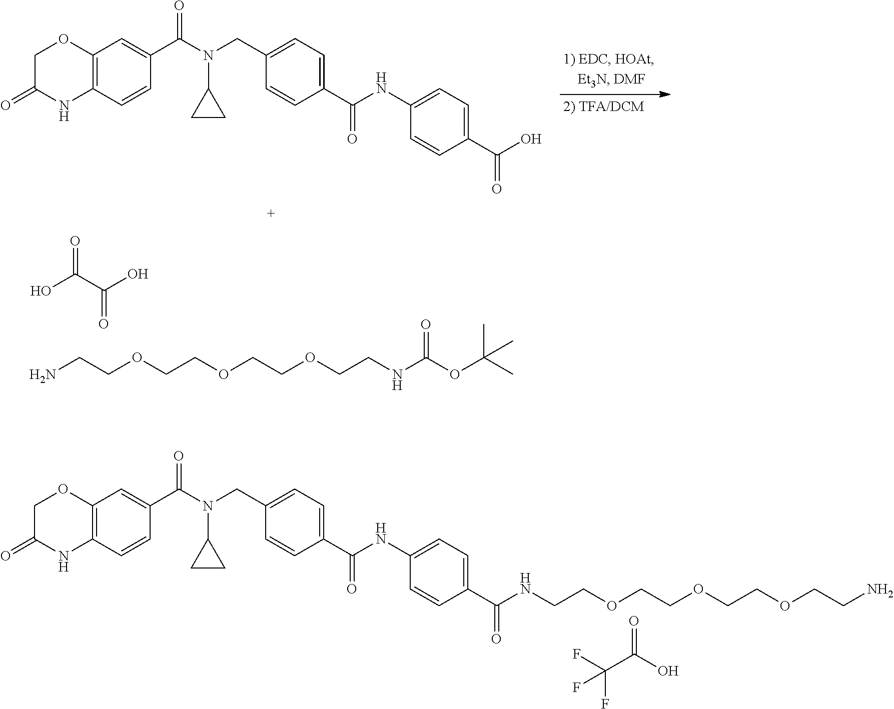

Prepared according to General Procedure A/Workup B using 4-(4-((N-cyclopropyl-3-oxo-3,4-dihydro-2H-benzo[b][1,4]oxazine-7-carboxamido)methyl)benzamido)benzoic acid (50.0 mg, 1 Eq, 103 μmol), EDC (29.6 mg, 1.5 Eq, 155 μmol), HOAt (21.0 mg, 1.5 Eq, 155 μmol), DMF (1 mL), tert-butyl (2-(2-(2-(2-aminoethoxy)ethoxy)ethoxy)ethyl)carbamate oxalate (39.4 mg, 1 Eq, 103 μmol) and triethylamine (41.7 mg, 57 μL, 4 Eq, 412 μmol). Following N-Boc deprotection, the product was purified by reverse phase chromatography (10-100% methanol in water+0.1% TFA) to give e N-(4-((4-((2-(2-(2-(2-aminoethoxy)ethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (7.68 mg, 9.93 μmol, 9.64%) as a white solid. ¹H NMR (400 MHz, cd₃od) δ 7.96 (d, J=8.2 Hz, 2H), 7.90-7.76 (m, 4H), 7.50 (d, J=7.7 Hz, 2H), 7.27-7.09 (m, 2H), 6.96 (d, J=8.1 Hz, 1H), 4.83 (s, 2H), 4.62 (s, 2H), 3.74-3.62 (m, 12H), 3.59 (t, J=5.4 Hz, 2H), 3.16-3.05 (m, 2H), 2.84 (s, 1H), 0.65 (s, 2H), 0.57 (s, 2H). LCMS (ESI, +ve mode) expected m/z for [C₃₅H₄₂N₅O₈⁺] [M+H] 660.3, found 660.2.

### Example 25

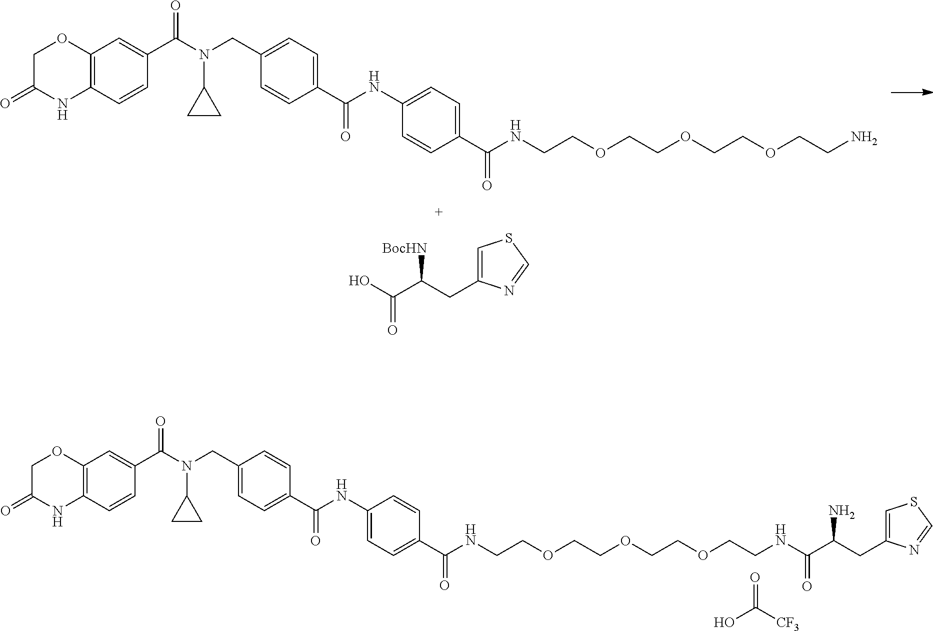

Prepared according to General Procedure B/Workup A using Boc-3-(4-Thiazolyl)-L-alanine (3.35 mg, 1.1 Eq, 12.3 μmol), TBTU (4.68 mg, 1.3 Eq, 14.6 μmol), DMF (0.5 mL), N-(4-((4-((2-(2-(2-(2-aminoethoxy)ethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (8.67 mg, 1 Eq, 11.2 μmol) and DIPEA (4.78 mg, 6.44 μL, 3.3 Eq, 37.0 μmol). Following N-Boc deprotection, the product was purified by reverse phase chromatography (10-100% methanol in water+0.1% TFA) to give (S)—N-(4-((4-((14-amino-13-oxo-15-(thiazol-4-yl)-3,6,9-trioxa-12-azapentadecyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (4.96 mg, 5.35 μmol, 47.7%) as a white solid. ¹H NMR (400 MHz, cd₃od) δ 9.00 (d, J=1.9 Hz, 1H), 7.96 (d, J=8.3 Hz, 2H), 7.91-7.75 (m, 4H), 7.50 (d, J=7.6 Hz, 2H), 7.43 (d, J=1.9 Hz, 1H), 7.29-7.06 (m, 2H), 6.96 (d, J=8.1 Hz, 1H), 4.83 (s, 2H), 4.63 (s, 2H), 4.22 (dd, J=7.6, 5.5 Hz, 1H), 3.72-3.61 (m, 8H), 3.61-3.54 (m, 4H), 3.54-3.36 (m, 4H), 3.34-3.32 (m, 1H), 3.30-3.25 (m, 1H), 2.84 (s, 1H), 0.76-0.62 (m, 2H), 0.62-0.31 (m, 2H). LCMS (ESI, +ve mode) expected m/z for [C₄₁H₄₈N₇O₉S⁺] [M+H] 814.3, found 814.2.

### Example 26

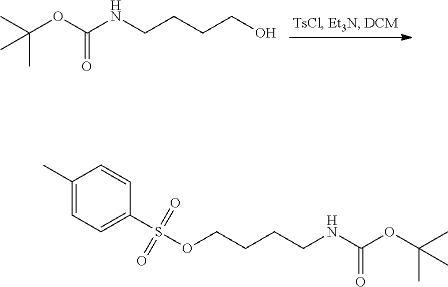

Prepared according to General Procedure F (Step 1) using tert-butyl (4-hydroxybutyl)carbamate (0.57 g, 1 Eq, 3.0 mmol), DCM (15 mL), triethylamine (0.61 g, 0.84 mL, 2 Eq, 6.0 mmol) and tosyl chloride (0.86 g, 1.5 Eq, 4.5 mmol). Normal phase chromatography over silica gel (0-25% EtOAc in hexanes) provided 4-((tert-butoxycarbonyl)amino)butyl 4-methylbenzenesulfonate (807 mg, 2.35 mmol, 78%). ¹H NMR (400 MHz, cdcl₃) δ 7.78 (d, J=8.3 Hz, 2H), 7.34 (d, J=8.1 Hz, 2H), 4.50 (s, 1H), 4.03 (t, J=6.3 Hz, 2H), 3.10-3.03 (m, 2H), 2.45 (s, 3H), 1.71-1.63 (m, 2H), 1.50 (dt, J=14.3, 7.1 Hz, 2H), 1.42 (s, 9H).

### Example 27

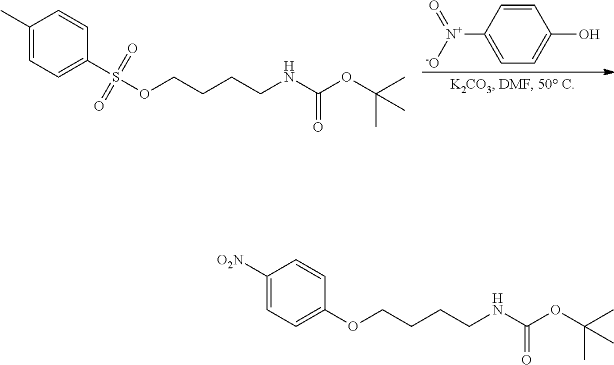

Prepared according to General Procedure E using 4-((tert-butoxycarbonyl)amino)butyl 4-methylbenzenesulfonate (400 mg, 1 Eq, 1.16 mmol), DMF (3 mL), 4-nitrophenol (211 mg, 1.3 Eq, 1.51 mmol) and potassium carbonate (241 mg, 1.5 Eq, 1.75 mmol). Normal phase chromatography over silica gel (0-25% EtOAc in hexanes) provided tert-butyl (4-(4-nitrophenoxy)butyl)carbamate (237.9 mg, 766.6 μmol, 65.8%) as a clear oil. ¹H NMR (400 MHz, cdcl₃) δ 8.19 (d, J=9.2 Hz, 2H), 6.94 (d, J=9.3 Hz, 2H), 4.59 (s, 1H), 4.07 (t, J=6.3 Hz, 2H), 3.32-3.04 (m, 2H), 1.95-1.77 (m, 2H), 1.68 (p, J=7.2 Hz, 2H), 1.44 (s, 9H). LCMS (ESI, +ve mode) expected m/z for [C₁₅H₂₂N₂O₅Na⁺] [M+Na] 333.1, found 333.2.

### Example 28

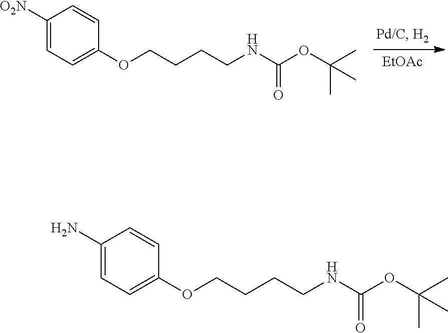 [SMILES: "EtOAc" ; CC(C)(C)OC(=O)NCCCCOc1ccc([N+](=O)[O-])cc1 ; CC(C)(C)OC(=O)NCCCCOc1ccc(N)cc1]

Prepared according to General Procedure C using tert-butyl (4-(4-nitrophenoxy)butyl)carbamate (237.9 mg, 1 Eq, 766.6 μmol), ethyl acetate (10 mL) and Pd/C (32.63 mg, 10% Wt, 0.04 Eq, 30.66 μmol) to give tert-butyl (4-(4-aminophenoxy)butyl)carbamate (204.3 mg 728.7 μmol, 95.06%). The crude material was used in the next step without purification. ¹H NMR (400 MHz, cdcl₃) δ 6.70-6.57 (m, 2H), 6.57-6.36 (m, 2H), 4.88 (s, 1H), 3.78 (t, J=6.2 Hz, 2H), 3.57-3.25 (m, 2H), 3.17-2.90 (m, 2H), 1.74-1.59 (m, 2H), 1.54 (p, J=6.8 Hz, 2H), 1.35 (s, 9H).

### Example 29

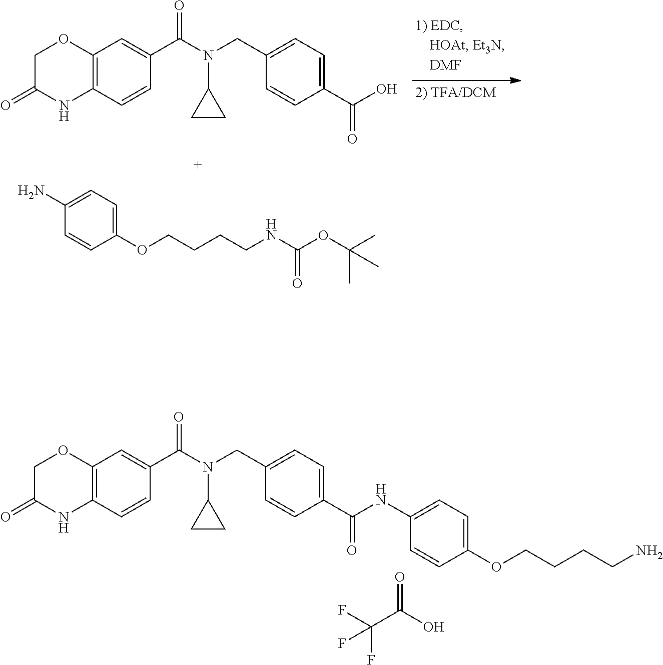

Prepared according to General Procedure A/Workup A using 4-((N-cyclopropyl-3-oxo-3,4-dihydro-2H-benzo[b][1,4]oxazine-7-carboxamido)methyl)benzoic acid (44 mg, 1 Eq, 0.12 mmol), EDC (34 mg, 1.5 Eq, 0.18 mmol), HOAt (24 mg, 1.5 Eq, 0.18 mmol), DMF (2 mL), tert-butyl (4-(4-aminophenoxy)butyl)carbamate (50 mg, 1.5 Eq, 0.18 mmol) and triethylamine (48 mg, 66 μL, 4 Eq, 0.48 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-(4-aminobutoxy)phenyl)carbamoyl)benzyl)-N-cyclopropyl-3-oxo-3,4-dihydro-2H-benzo[b][1,4]oxazine-7-carboxamide 2,2,2-trifluoroacetate (8.04 mg, 12.5 μmol, 11%) as a white solid (8.04 mg, 12.5 μmol, 11%). ¹H NMR (400 MHz, cd₃od) δ 7.93 (d, J=8.2 Hz, 2H), 7.57 (d, J=9.0 Hz, 2H), 7.48 (d, J=7.5 Hz, 2H), 7.28-7.08 (m, 2H), 7.01-6.83 (m, 3H), 4.82 (s, 2H), 4.62 (s, 2H), 4.05 (t, J=5.4 Hz, 2H), 3.02 (t, J=6.6 Hz, 2H), 2.82 (s, 1H), 2.02-1.70 (m, 4H), 0.64 (s, 2H), 0.56 (s, 2H). LCMS (ESI, +ve mode) expected m/z for [C₃₀H₃₃N₄O₅⁺] [M+H] 529.2, found 529.2.

### Example 30

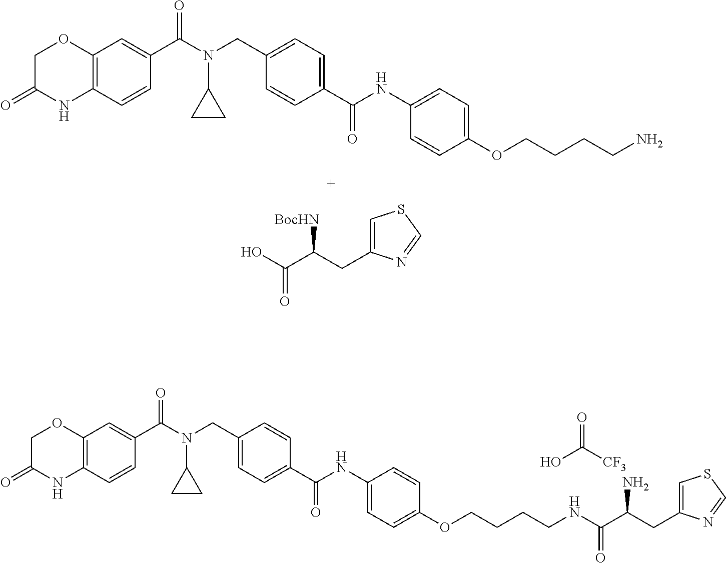

Prepared according to General Procedure B/Workup A using Boc-3-(4-Thiazolyl)-L-alanine (9.3 mg, 1.1 Eq, 34 μmol), TBTU (13 mg, 1.3 Eq, 40 μmol), DMF (1 mL), N-(4-((4-(4-aminobutoxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 31 μmol) and DIPEA (13 mg, 18 μL, 3.3 Eq, 0.10 mmol). Following N-Boc deprotection, the product was purified by reverse phase chromatography (10-100% methanol in water+0.1% TFA) to give (S)—N-(4-((4-(4-(2-amino-3-(thiazol-4-yl)propanamido)butoxy)phenyl)carbamoyl)benzyl)-N-cyclopropyl-3-oxo-3,4-dihydro-2H-benzo[b][1,4]oxazine-7-carboxamide 2,2,2-trifluoroacetate (4.01 mg, 5.03 μmol, 16%) as a white solid. ¹H NMR (400 MHz, cd₃od) δ 9.01 (d, J=1.9 Hz, 1H), 7.93 (d, J=8.2 Hz, 2H), 7.56 (d, J=9.0 Hz, 2H), 7.48 (d, J=7.4 Hz, 2H), 7.42 (d, J=1.9 Hz, 1H), 7.28-7.10 (m, 2H), 6.97 (d, J=8.1 Hz, 1H), 6.92 (d, J=9.0 Hz, 2H), 4.82 (s, 2H), 4.62 (s, 2H), 4.19 (dd, J=7.5, 5.9 Hz, 1H), 4.08-3.85 (m, 2H), 3.44-3.35 (m, 2H), 3.30-3.12 (m, 2H), 2.83 (s, 1H), 1.81-1.69 (m, 2H), 1.69-1.54 (m, 2H), 0.79-0.61 (m, 2H), 0.61-0.34 (m, 2H). LCMS (ESI, +ve mode) expected m/z for [C₃₆H₃₉N₆O₆S⁺] [M+H] 683.3, found 683.2.

### Example 31

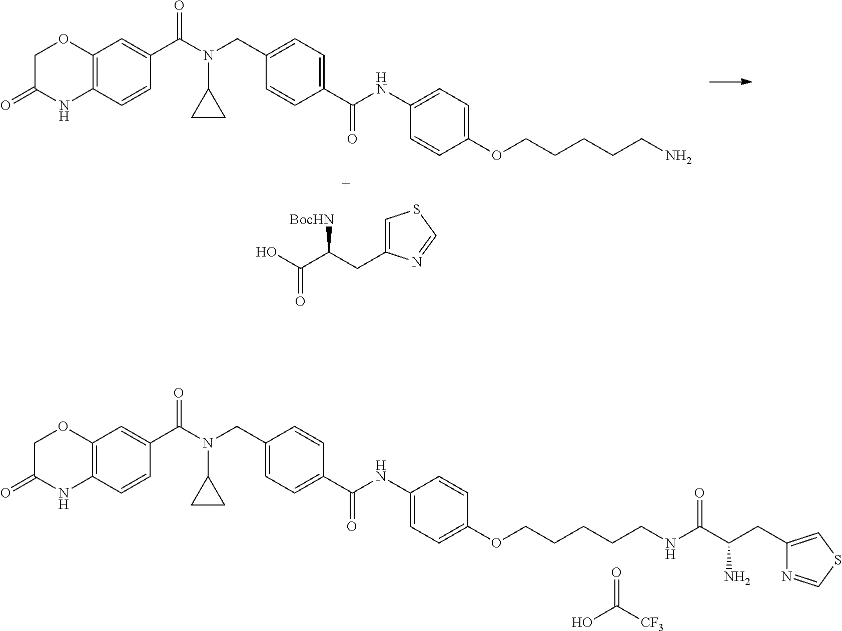

Prepared according to General Procedure B/Workup A using Boc-3-(4-Thiazolyl)-L-alanine (9.1 mg, 1.1 Eq, 34 μmol), TBTU (13 mg, 1.3 Eq, 40 μmol), DMF (1 mL), N-(4-((4-((5-aminopentyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 30 μmol) and DIPEA (13 mg, 18 μL, 3.3 Eq, 0.10 mmol). Following N-Boc deprotection, the product was purified by reverse phase chromatography (10-100% methanol in water+0.1% TFA) to give (S)—N-(4-((4-((5-(2-amino-3-(thiazol-4-yl)propanamido)pentyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (16.7 mg, 20.6 μmol, 68%) as a white solid. ¹H NMR (400 MHz, cd₃od) δ 9.01 (d, J=1.9 Hz, 1H), 7.93 (d, J=8.2 Hz, 2H), 7.55 (d, J=9.0 Hz, 2H), 7.48 (d, J=7.6 Hz, 2H), 7.41 (d, J=1.9 Hz, 1H), 7.27-7.10 (m, 2H), 6.96 (d, J=8.0 Hz, 1H), 6.91 (d, J=9.0 Hz, 2H), 4.82 (s, 2H), 4.62 (s, 2H), 4.19 (dd, J=7.6, 5.8 Hz, 1H), 3.98 (t, J=6.2 Hz, 2H), 3.44-3.32 (m, 2H), 3.29-3.23 (m, 1H), 3.23-3.06 (m, 1H), 2.82 (s, 1H), 1.78 (p, J=6.4 Hz, 2H), 1.60-1.35 (m, 4H), 0.88-0.60 (m, 2H), 0.61-0.36 (m, 2H). LCMS (ESI, +ve mode) expected m/z for [C₃₇H₄₁N₆O₆S⁺] [M+H] 697.3, found 697.2.

### Example 32

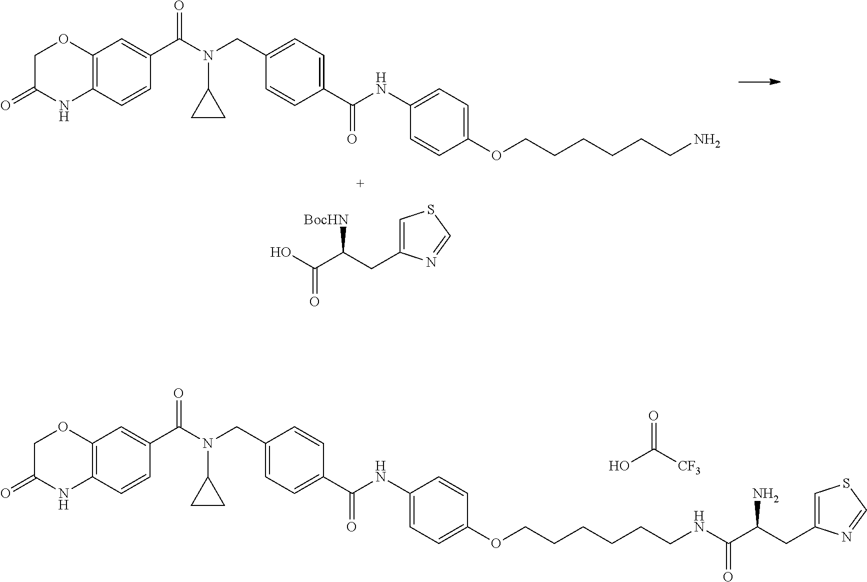 [SMILES: NCCCCCCOc1ccc(NC(=O)c2ccc(CN(C(=O)c3ccc4c(c3)OCC(=O)N4)C3CC3)cc2)cc1 ; CC(C)(C)OC(=O)N[C@@H](Cc1cscn1)C(=O)O ; N[C@@H](Cc1cscn1)C(=O)NCCCCCCOc1ccc(NC(=O)c2ccc(CN(C(=O)c3ccc4c(c3)OCC(=O)N4)C3CC3)cc2)cc1 ; O=C(O)C(F)(F)F]

Prepared according to General Procedure B/Workup A using Boc-3-(4-Thiazolyl)-L-alanine (8.9 mg, 1.1 Eq, 33 μmol), TBTU (12 mg, 1.3 Eq, 39 μmol), DMF (1 mL), N-(4-((4-((6-aminohexyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 30 μmol) and DIPEA (13 mg, 17 μL, 3.3 Eq, 98 μmol). Following N-Boc deprotection, the product was purified by reverse phase chromatography (10-100% methanol in water+0.1% TFA) to give (S)—N-(4-((4-((6-(2-amino-3-(thiazol-4-yl)propanamido)hexyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (2.04 mg, 2.47 μmol, 8.3%) as a white solid. ¹H NMR (400 MHz, cd₃od) δ 9.01 (d, J=1.9 Hz, 1H), 7.93 (d, J=8.2 Hz, 2H), 7.55 (d, J=8.9 Hz, 2H), 7.48 (d, J=7.5 Hz, 2H), 7.41 (d, J=1.8 Hz, 1H), 7.25-7.06 (m, 2H), 6.96 (d, J=8.0 Hz, 1H), 6.92 (d, J=9.0 Hz, 2H), 4.83 (s, 2H), 4.62 (s, 2H), 4.18 (dd, J=7.6, 5.8 Hz, 1H), 3.99 (t, J=6.3 Hz, 2H), 3.43-3.32 (m, 2H), 3.28-3.21 (m, 1H), 3.14 (dt, J=13.4, 6.8 Hz, 1H), 2.82 (s, 1H), 1.78 (p, J=6.5 Hz, 2H), 1.61-1.42 (m, 4H), 1.42-1.31 (m, 2H), 0.80-0.61 (m, 2H), 0.61-0.38 (m, 2H). LCMS (ESI, +ve mode) expected m/z for [C₃₈H₄₃N₆O₆S⁺] [M+H] 711.3, found 711.2.

### Example 33

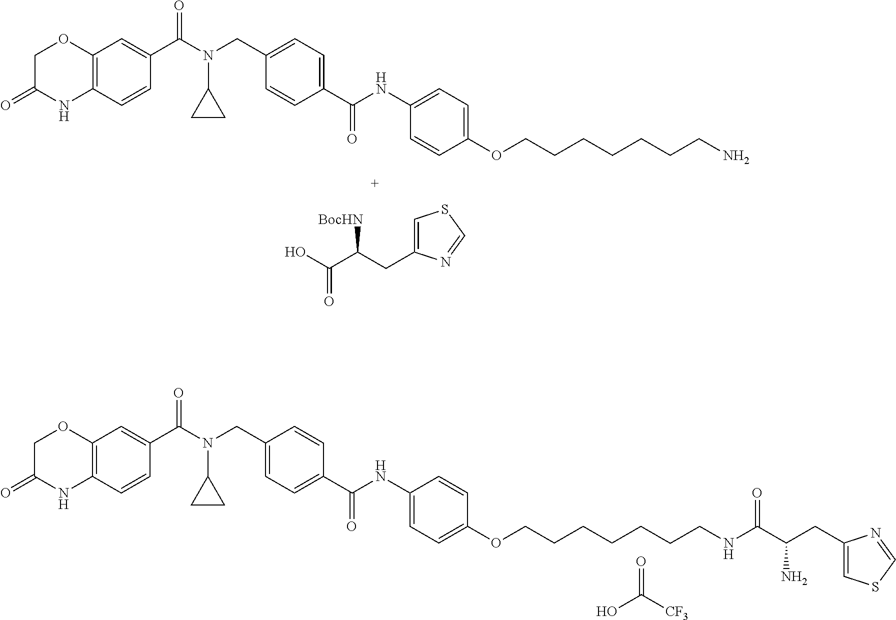

Prepared according to General Procedure B/Workup A using Boc-3-(4-Thiazolyl)-L-alanine (8.7 mg, 1.1 Eq, 32 μmol), TBTU (12 mg, 1.3 Eq, 38 μmol), DMF (1 mL), N-(4-((4-((7-aminoheptyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 29 μmol), DIPEA (12 mg, 17 μL, 3.3 Eq, 96 μmol). Following N-Boc deprotection, the product was purified by reverse phase chromatography (10-100% methanol in water+0.1% TFA) to give (S)—N-(4-((4-((7-(2-amino-3-(thiazol-4-yl)propanamido)heptyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (6.51 mg, 7.76 μmol, 27%) as a white solid. ¹H NMR (400 MHz, cd₃od) δ 8.99 (d, J=1.9 Hz, 1H), 7.91 (d, J=8.2 Hz, 2H), 7.53 (d, J=9.0 Hz, 2H), 7.46 (d, J=7.3 Hz, 2H), 7.39 (d, J=1.9 Hz, 1H), 7.23-7.10 (m, 2H), 6.94 (d, J=8.1 Hz, 1H), 6.89 (d, J=9.0 Hz, 2H), 4.80 (s, 2H), 4.60 (s, 2H), 4.17 (dd, J=7.6, 5.9 Hz, 1H), 3.96 (t, J=6.3 Hz, 2H), 3.41-3.30 (m, 2H), 3.27-3.17 (m, 1H), 3.11 (dt, J=13.5, 6.9 Hz, 1H), 2.80 (s, 1H), 1.76 (p, J=6.5 Hz, 2H), 1.54-1.42 (m, 4H), 1.42-1.34 (m, 2H), 1.34-1.27 (m, 2H), 0.73-0.58 (m, 2H), 0.58-0.43 (m, 2H). LCMS (ESI, +ve mode) expected m/z for [C₃₉H₄₅N₆O₆S⁺] [M+H] 725.3, found 725.2.

### Example 34

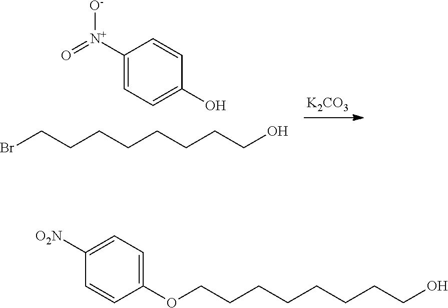

Prepared according to General Procedure E using 8-bromooctan-1-ol (0.50 g, 1 Eq, 2.4 mmol), 4-nitrophenol (0.40 g, 1.2 Eq, 2.9 mmol), potassium carbonate (0.99 g, 3 Eq, 7.2 mmol), and DMF (3 mL). Normal phase chromatography over silica gel (0-50% EtOAc in hexanes) provided provided 8-(4-nitrophenoxy)octan-1-ol (536.5 mg, 2.007 mmol, 84%) as a white solid. ¹H NMR (400 MHz, cdcl₃) δ 8.19 (d, J=9.3 Hz, 2H), 6.94 (d, J=9.3 Hz, 2H), 4.04 (t, J=6.5 Hz, 2H), 3.78-3.55 (m, 2H), 1.88-1.75 (m, 2H), 1.62-1.54 (m, 2H), 1.52-1.42 (m, 2H), 1.42-1.34 (m, 6H). LCMS (ESI, +ve mode) expected m/z for [C₁₄H₂₂NO₄⁺] [M+H] 268.2, found 268.1.

### Example 35

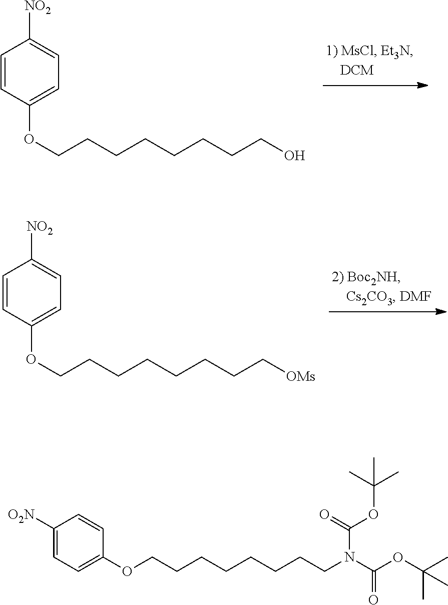 [SMILES: O=[N+]([O-])c1ccc(OCCCCCCCCO)cc1 ; CS(=O)(=O)OCCCCCCCCOc1ccc([N+](=O)[O-])cc1 ; CC(C)(C)OC(=O)N(CCCCCCCCOc1ccc([N+](=O)[O-])cc1)C(=O)OC(C)(C)C]

Prepared according to General Procedure F using (Step 1) 8-(4-nitrophenoxy)octan-1-ol (536.5 mg, 1 Eq, 2.007 mmol), DCM (10 mL), triethylamine (365.5 mg, 503 μL, 1.8 Eq, 3.612 mmol), mesyl chloride (321.8 mg, 218.9 μL, 1.4 Eq, 2.810 mmol) and (Step 2) di-tert-butyl iminodicarbonate (523.2 mg, 1.2 Eq, 2.408 mmol), Cs₂CO₃ (980.8 mg, 1.5 Eq, 3.010 mmol) and DMF (5 mL). Normal phase chromatography over silica gel (0-25% EtOAc in hexanes) provided tert-butyl (tert-butoxycarbonyl)(8-(4-nitrophenoxy)octyl)carbamate (848.7 mg, 1.819 mmol, 90.64%) as a clear oil. ¹H NMR (400 MHz, cdcl₃) δ 8.19 (d, J=9.2 Hz, 2H), 6.93 (d, J=9.2 Hz, 2H), 4.04 (t, J=6.5 Hz, 2H), 3.60-3.40 (m, 2H), 1.81 (dt, J=14.4, 6.6 Hz, 2H), 1.62-1.54 (m, 2H), 1.54-1.50 (m, 2H), 1.50 (s, 18H), 1.40-1.30 (m, 6H).

### Example 36

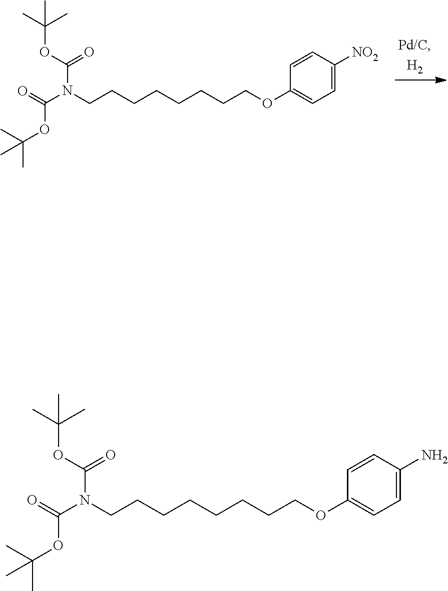 [SMILES: CC(C)(C)OC(=O)N(CCCCCCCCOc1ccc([N+](=O)[O-])cc1)C(=O)OC(C)(C)C ; CC(C)(C)OC(=O)N(CCCCCCCCOc1ccc(N)cc1)C(=O)OC(C)(C)C]

Prepared according to General Procedure C using tert-butyl (tert-butoxycarbonyl)(8-(4-nitrophenoxy)octyl)carbamate (848.7 mg, 1 Eq, 1.819 mmol), ethyl acetate (24 mL) and Pd/C (77.43 mg, 10% Wt, 0.04 Eq, 72.76 μmol) to give a ruby, red oil. The crude material was purified by normal phase chromatography over silica gel (0-10% MeOH in DCM) to give tert-butyl (8-(4-aminophenoxy)octyl)(tert-butoxycarbonyl)carbamate (393.2 mg, 900.6 μmol, 49.51%) as a dark red oil. ¹H NMR (400 MHz, cdcl₃) δ 6.73 (d, J=8.8 Hz, 2H), 6.63 (d, J=8.8 Hz, 2H), 3.87 (t, J=6.6 Hz, 2H), 3.58-3.51 (m, 2H), 3.42 (s, 2H), 1.73 (dt, J=14.5, 6.6 Hz, 2H), 1.59-1.54 (m, 2H), 1.50 (s, 18H), 1.45-1.39 (m, 2H), 1.37-1.29 (m, 6H).

### Example 37

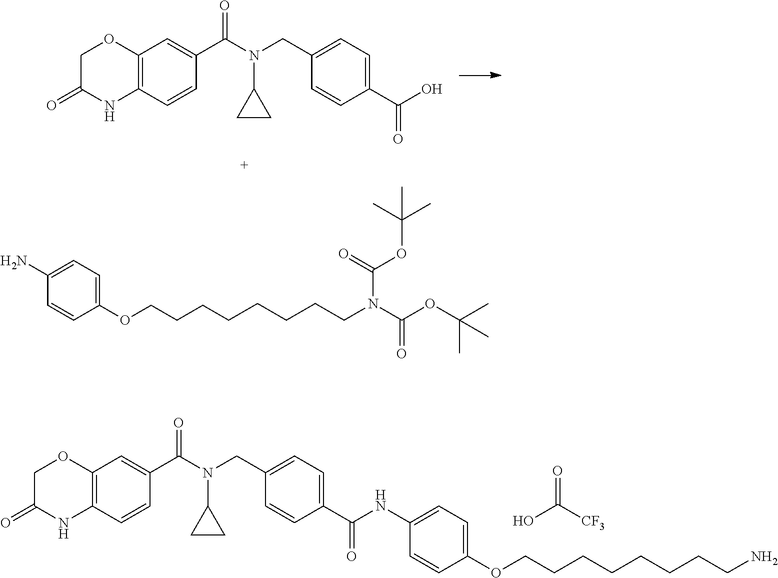

Prepared according to General Procedure A/Workup A using 4-((N-cyclopropyl-3-oxo-3,4-dihydro-2H-benzo[b][1,4]oxazine-7-carboxamido)methyl)benzoic acid (173.7 mg, 1 Eq, 474.0 μmol), EDC (136.3 mg, 1.5 Eq, 711.0 μmol), HOAt (96.78 mg, 1.5 Eq, 711.0 μmol), DMF (4 mL), tert-butyl (8-(4-aminophenoxy)octyl)(tert-butoxycabonyl)carbamate (393.2 mg, 1.9 Eq, 900.6 μmol) and triethylamine (191.9 mg, 0.26 mL, 4 Eq, 1.896 mmol). The N-Boc protected product was purified by normal phase chromatography over silica gel (0-10% MeOH in DCM). Following N-Boc deprotection, the product was purified twice by reverse phase chromatography (10-100% methanol in water+0.1% TFA) to give N-(4-((4-((8-aminooctyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (71.56 mg, 102.4 μmol, 21.61%) as a white solid. ¹H NMR (400 MHz, cd₃od) δ 7.92 (d, J=8.0 Hz, 2H), 7.56 (d, J=8.8 Hz, 2H), 7.43 (d, J=7.0 Hz, 2H), 7.24-7.08 (m, 2H), 6.94 (d, J=8.0 Hz, 1H), 6.88 (d, J=8.9 Hz, 2H), 4.77 (s, 2H), 4.58 (s, 2H), 3.93 (t, J=6.3 Hz, 2H), 2.89 (t, J=7.6 Hz, 2H), 2.77 (s, 1H), 1.74 (dt, J=14.1, 6.5 Hz, 2H), 1.68-1.55 (m, 2H), 1.55-1.43 (m, 2H), 1.41-1.31 (m, 6H), 0.79-0.56 (m, 2H), 0.56-0.27 (m, 2H). LCMS (ESI, +ve mode) expected m/z for [C₃₄H₄₁N₄O₅⁺] [M+H] 585.3, found 585.3.

### Example 38

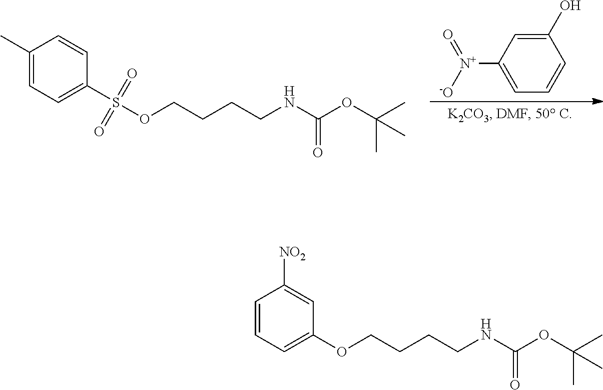

Prepared according to General Procedure E using 4-((tert-butoxycarbonyl)amino)butyl 4-methylbenzenesulfonate (400 mg, 1 Eq, 1.16 mmol), DMF (3 mL), 3-nitrophenol (211 mg, 1.3 Eq, 1.51 mmol) and potassium carbonate (241 mg, 1.5 Eq, 1.75 mmol). Normal phase chromatography over silica gel (0-25% EtOAc in hexanes) provided tert-butyl (4-(3-nitrophenoxy)butyl)carbamate (190 mg, 612 μmol, 52.6%) as a clear oil. ¹H NMR (400 MHz, cdcl₃) δ 7.73 (ddd, J=8.1, 2.0, 0.7 Hz, 1H), 7.63 (t, J=2.3 Hz, 1H), 7.35 (t, J=8.2 Hz, 1H), 7.20-7.08 (m, 1H), 4.73 (s, 1H), 4.00 (t, J=6.2 Hz, 2H), 3.25 (t, J=6.8 Hz, 1H), 3.15 (t, J=7.0 Hz, 2H), 1.84-1.74 (m, 2H), 1.70-1.55 (m, 2H), 1.38 (s, 9H).

### Example 39

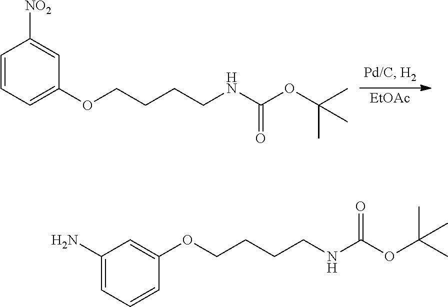 [SMILES: "EtOAc" ; CC(C)(C)OC(=O)NCCCCOc1cccc([N+](=O)[O-])c1 ; CC(C)(C)OC(=O)NCCCCOc1cccc(N)c1]

Prepared according to General Procedure C using tert-butyl (4-(3-nitrophenoxy)butyl)carbamate (190 mg, 1 Eq, 612 μmol), ethyl acetate (10 mL) and Pd/C (26.1 mg, 10% Wt, 0.04 Eq, 24.5 μmol) to give tert-butyl (4-(3-aminophenoxy)butyl)carbamate (171 mg, 610 μmol, 99.6%). The crude material was used in the next step without purification. ¹H NMR (400 MHz, cdcl₃) δ 6.93 (t, J=8.0 Hz, 1H), 6.30-6.16 (m, 2H), 6.14 (t, J=2.1 Hz, 1H), 4.88 (s, 1H), 3.82 (t, J=6.2 Hz, 2H), 3.61 (s, 2H), 3.14-3.00 (m, 2H), 1.83-1.61 (m, 2H), 1.55 (dt, J=13.7, 6.8 Hz, 2H), 1.36 (s, 9H).

### Example 40

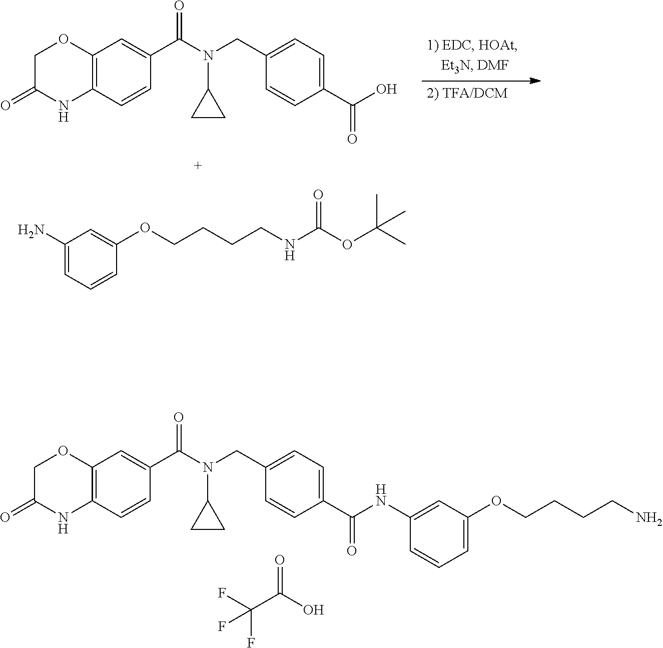

Prepared according to General Procedure A/Workup A using 4-((N-cyclopropyl-3-oxo-3,4-dihydro-2H-benzo[b][1,4]oxazine-7-carboxamido)methyl)benzoic acid (44 mg, 1 Eq, 0.12 mmol), EDC (34 mg, 1.5 Eq, 0.18 mmol), HOAt (24 mg, 1.5 Eq, 0.18 mmol), DMF (1.5 mL) tert-butyl (4-(3-aminophenoxy)butyl)carbamate (50 mg, 1.5 Eq, 0.18 mmol) and triethylamine (48 mg, 66 μL, 4 Eq, 0.48 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((3-(4-aminobutoxy)phenyl)carbamoyl)benzyl)-N-cyclopropyl-3-oxo-3,4-dihydro-2H-benzo[b][1,4]oxazine-7-carboxamide 2,2,2-trifluoroacetate (6.65 mg, 10.3 μmol, 8.7%) as a white solid. ¹H NMR (400 MHz, cd₃od) δ 7.92 (d, J=8.2 Hz, 2H), 7.63-7.31 (m, 3H), 7.24 (t, J=8.0 Hz, 1H), 7.21-7.02 (m, 3H), 6.95 (d, J=8.1 Hz, 1H), 6.78-6.51 (m, 1H), 4.80 (s, 2H), 4.60 (s, 2H), 4.04 (t, J=5.3 Hz, 2H), 3.02 (d, J=6.7 Hz, 2H), 2.80 (s, 1H), 1.99-1.71 (m, 4H), 1.01-0.58 (m, 2H), 0.58-0.12 (m, 2H). LCMS (ESI, +ve mode) expected m/z for [C₃₀H₃₃N₄O₅⁺] [M+H] 529.2, found 529.3.

### Example 41

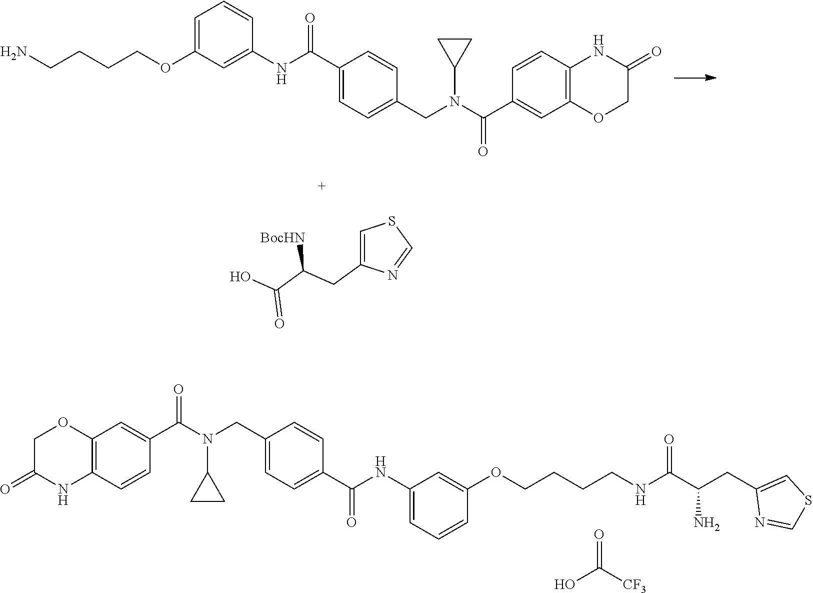 [SMILES: NCCCCOc1cccc(NC(=O)c2ccc(CN(C(=O)c3ccc4c(c3)OCC(=O)N4)C3CC3)cc2)c1 ; CC(C)(C)OC(=O)N[C@@H](Cc1cscn1)C(=O)O ; N[C@@H](Cc1cscn1)C(=O)NCCCCOc1cccc(NC(=O)c2ccc(CN(C(=O)c3ccc4c(c3)OCC(=O)N4)C3CC3)cc2)c1 ; O=C(O)C(F)(F)F]

Prepared according to General Procedure B/Workup A using Boc-3-(4-Thiazolyl)-L-alanine (9.3 mg, 1.1 Eq, 34 μmol), TBTU (13 mg, 1.3 Eq, 40 μmol), DMF (1 mL), N-(4-((3-(4-aminobutoxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 31 μmol) and DIPEA (13 mg, 18 μL, 3.3 Eq, 0.10 mmol). Following N-Boc deprotection, the product was purified by reverse phase chromatography (10-100% methanol in water+0.1% TFA) to give (S)—N-(4-((3-(4-(2-amino-3-(thiazol-4-yl)propanamido)butoxy)phenyl)carbamoyl)benzyl)-N-cyclopropyl-3-oxo-3,4-dihydro-2H-benzo[b][1,4]oxazine-7-carboxamide 2,2,2-trifluoroacetate (10.21 mg, 12.81 μmol, 41%) as a white solid. ¹H NMR (400 MHz, cd₃od) δ 9.00 (d, J=1.9 Hz, 1H), 7.93 (d, J=8.2 Hz, 2H), 7.62-7.44 (m, 3H), 7.41 (d, J=1.8 Hz, 1H), 7.24 (t, J=8.1 Hz, 1H), 7.22-7.08 (m, 3H), 6.96 (d, J=8.1 Hz, 1H), 6.71 (dd, J=8.2, 1.7 Hz, 1H), 4.82 (s, 2H), 4.62 (s, 2H), 4.21 (dd, J=7.5, 5.9 Hz, 1H), 4.00 (t, J=6.1 Hz, 2H), 3.43-3.35 (m, 2H), 3.30-3.26 (m, 1H), 3.26-3.11 (m, 1H), 2.83 (s, 1H), 1.86-1.70 (m, 2H), 1.70-1.55 (m, 2H), 0.81-0.60 (m, 2H), 0.60-0.27 (m, 2H). LCMS (ESI, +ve mode) expected m/z for [C₃₆H₃₉N₆O₆S⁺] [M+H] 683.3, found 683.2.

### Example 42

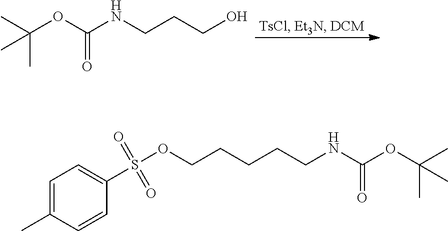

Prepared according to General Procedure F (Step 1) using tert-butyl (5-hydroxypentyl)carbamate (0.61 g, 1 Eq, 3.0 mmol), DCM (15 mL), triethylamine (0.61 g, 0.84 mL, 2 Eq, 6.0 mmol) and tosyl chloride (0.86 g, 1.5 Eq, 4.5 mmol). Normal phase chromatography over silica gel (0-25% EtOAc in hexanes) provided 5-((tert-butoxycarbonyl)amino)pentyl 4-methylbenzenesulfonate (1.0336 g, 2.8914 mmol, 96%) as a clear oil. ¹H NMR (400 MHz, cdcl₃) δ 7.78 (d, J=8.3 Hz, 2H), 7.34 (d, J=8.0 Hz, 2H), 4.49 (s, 1H), 4.01 (t, J=6.4 Hz, 2H), 3.16-2.92 (m, 2H), 2.45 (s, 3H), 1.65 (dt, J=13.9, 6.5 Hz, 2H), 1.43 (s, 9H), 1.42-1.38 (m, 2H), 1.38-1.28 (m, 3H). LCMS (ESI, +ve mode) expected m/z for [C₁₇H₂₇NO₅SNa⁺] [M+Na] 380.2 found 380.1.

### Example 43

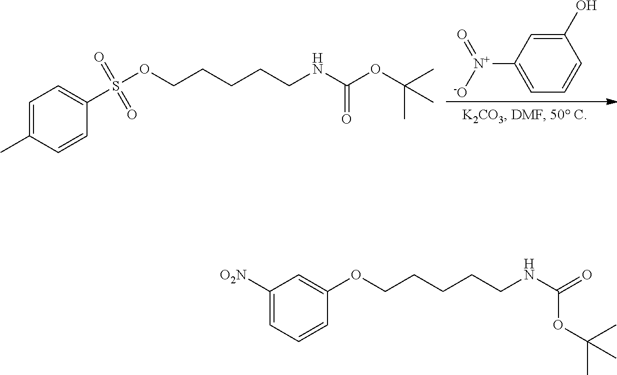

Prepared according to General Procedure E using 5-((tert-butoxycarbonyl)amino)pentyl 4-methylbenzenesulfonate (500 mg, 1 Eq, 1.40 mmol), DMF (3 mL), 3-nitrophenol (253 mg, 1.3 Eq, 1.82 mmol) and potassium carbonate (290 mg, 1.5 Eq, 2.10 mmol). Normal phase chromatography over silica gel (0-25% EtOAc in hexanes) provided tert-butyl (5-(3-nitrophenoxy)pentyl)carbamate (299.2 mg, 922.4 μmol, 65.90%) as a clear oil. ¹H NMR (400 MHz, cdcl₃) δ 7.87-7.76 (m, 1H), 7.71 (t, J=2.2 Hz, 1H), 7.41 (t, J=8.2 Hz, 1H), 7.20 (dd, J=8.3, 2.4 Hz, 1H), 4.03 (t, J=6.3 Hz, 2H), 3.15 (s, 2H), 1.84 (p, J=6.5 Hz, 2H), 1.68-1.47 (m, 5H), 1.44 (s, 9H). LCMS (ESI, +ve mode) expected m/z for [C₁₆H₂₄N₂O₅Na⁺] [M+Na] 347.1, found 347.2.

### Example 44

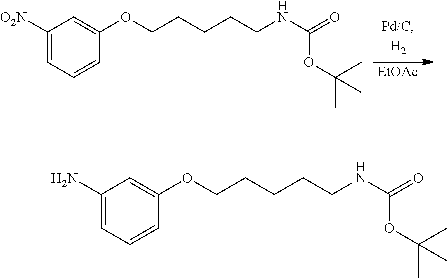 [SMILES: "EtOAc" ; CC(C)(C)OC(=O)NCCCCCOc1cccc([N+](=O)[O-])c1 ; CC(C)(C)OC(=O)NCCCCCOc1cccc(N)c1]

Prepared according to General Procedure C using tert-butyl (5-(3-nitrophenoxy)pentyl)carbamate (299.2 mg, 1 Eq, 922.4 μmol), ethyl acetate (10 mL) and Pd/C (39.26 mg, 10% Wt, 0.04 Eq, 36.89 μmol) to give tert-butyl (5-(3-aminophenoxy)pentyl)carbamate (270 mg 917 μmol, 99.4%). The crude material was used in the next step without purification. ¹H NMR (400 MHz, cdcl₃) δ 6.97 (t, J=8.0 Hz, 1H), 6.41-6.20 (m, 2H), 6.18 (t, J=2.1 Hz, 1H), 4.74 (s, 1H), 3.84 (t, J=6.4 Hz, 2H), 3.58 (s, 2H), 3.16-2.92 (m, 2H), 1.71 (p, J=6.6 Hz, 2H), 1.57-1.41 (m, 4H), 1.40 (s, 9H). LCMS (ESI, +ve mode) expected m/z for [C₁₆H₂₇N₂O₃⁺] [M+H] 295.2, found 295.2.

### Example 45

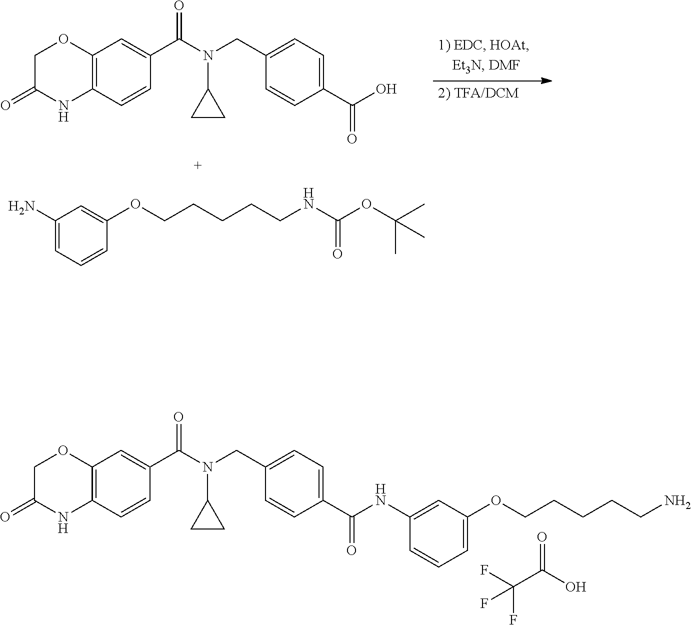

Prepared according to General Procedure A/Workup A using 4-((N-cyclopropyl-3-oxo-3,4-dihydro-2H-benzo[b][1,4]oxazine-7-carboxamido)methyl)benzoic acid (37 mg, 1 Eq, 0.10 mmol), EDC (29 mg, 1.5 Eq, 0.15 mmol), HOAt (20 mg, 1.5 Eq, 0.15 mmol), DMF (4 mL) tert-butyl (5-(3-aminophenoxy)pentyl)carbamate (50 mg, 1.7 Eq, 0.17 mmol) and triethylamine (40 mg, 55 μL, 4 Eq, 0.40 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((3-((5-aminopentyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (6.18 mg, 9.41 μmol, 9.4%) as a white solid. ¹H NMR (400 MHz, cd₃od) δ 7.89 (d, J=8.4 Hz, 2H), 7.51-7.29 (m, 3H), 7.29-7.14 (m, 2H), 7.17-7.02 (m, 2H), 6.92 (d, J=8.0 Hz, 1H), 6.67 (dt, J=6.5, 2.6 Hz, 1H), 4.75 (s, 2H), 4.56 (s, 2H), 3.96 (t, J=6.2 Hz, 2H), 2.91 (t, J=7.6 Hz, 2H), 2.75 (d, J=8.9 Hz, 1H), 1.85-1.72 (m, 2H), 1.76-1.63 (m, 2H), 1.59-1.45 (m, 2H), 0.76-0.54 (m, 2H), 0.54-0.31 (m, 2H). LCMS (ESI, +ve mode) expected m/z for [C₃₁H₃₅N₄O₅⁺] [M+H] 543.3, found 543.2.

### Example 46

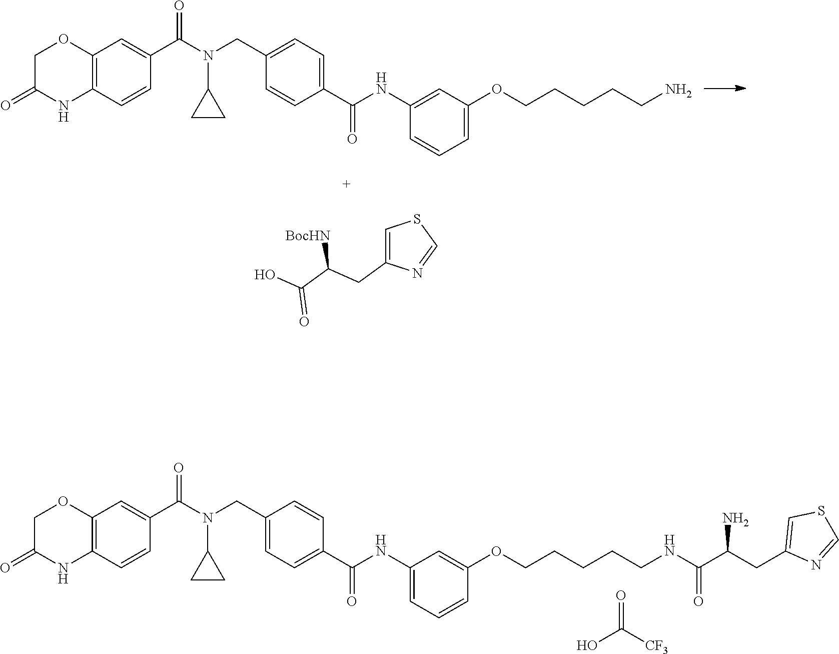

Prepared according to General Procedure B/Workup A using Boc-3-(4-Thiazolyl)-L-alanine (9.1 mg, 1.1 Eq, 34 μmol), TBTU (13 mg, 1.3 Eq, 40 μmol), DMF (1 mL), N-(4-((3-((5-aminopentyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 30 μmol) and DIPEA (13 mg, 18 μL, 3.3 Eq, 0.10 mmol). Following N-Boc deprotection, the product was purified by reverse phase chromatography (10-100% methanol in water+0.1% TFA) to give (S)—N-(4-((3-((5-(2-amino-3-(thiazol-4-yl)propanamido)pentyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (4.37 mg, 5.39 μmol, 18%) as a white solid. ¹H NMR (400 MHz, cd₃od) δ 9.00 (d, J=1.9 Hz, 1H), 7.93 (d, J=8.2 Hz, 2H), 7.58-7.43 (m, 3H), 7.41 (d, J=1.8 Hz, 1H), 7.24 (t, J=8.1 Hz, 1H), 7.22-7.07 (m, 3H), 6.96 (d, J=8.1 Hz, 1H), 6.71 (dd, J=8.2, 1.8 Hz, 1H), 4.83 (s, 2H), 4.62 (s, 2H), 4.20 (dd, J=7.5, 5.9 Hz, 1H), 4.00 (t, J=6.2 Hz, 2H), 3.45-3.32 (m, 2H), 3.30-3.22 (m, 1H), 3.16 (dt, J=13.3, 6.3 Hz, 1H), 2.83 (s, 1H), 1.88-1.66 (m, 2H), 1.61-1.32 (m, 4H), 0.76-0.61 (m, 2H), 0.61-0.35 (m, 2H). LCMS (ESI, +ve mode) expected m/z for [C₃₇H₄₁N₆O₆S⁺] [M+H] 697.3, found 697.2.

### Example 47

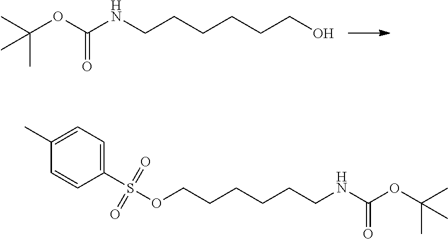 [SMILES: CC(C)(C)OC(=O)NCCCCCCO ; Cc1ccc(S(=O)(=O)OCCCCCCNC(=O)OC(C)(C)C)cc1]

Prepared according to General Procedure F (Step 1) using tert-butyl (6-hydroxyhexyl)carbamate (343 mg, 1 Eq, 1.58 mmol), DCM (15 mL), tosyl chloride (451 mg, 1.5 Eq, 2.37 mmol) and triethylamine (319 mg, 440 μL, 2 Eq, 3.16 mmol). Normal phase chromatography over silica gel (0-25% EtOAc in hexanes) provided 6-((tert-butoxycarbonyl)amino)hexyl 4-methylbenzenesulfonate (580 mg, 1.56 mmol, 98.9%) as a clear oil. ¹H NMR (400 MHz, cdcl₃) δ 7.78 (d, J=8.3 Hz, 2H), 7.34 (d, J=8.0 Hz, 2H), 4.49 (s, 1H), 4.01 (t, J=6.5 Hz, 2H), 3.19-2.88 (m, 2H), 2.45 (s, 3H), 1.63 (p, J=6.5 Hz, 3H), 1.43 (s, 9H), 1.42-1.36 (m, 2H), 1.36-1.26 (m, 4H). LCMS (ESI, +ve mode) expected m/z for [C₁₈H₂₉NO₅SNa⁺] [M+Na] 394.2, found 394.2.

### Example 48

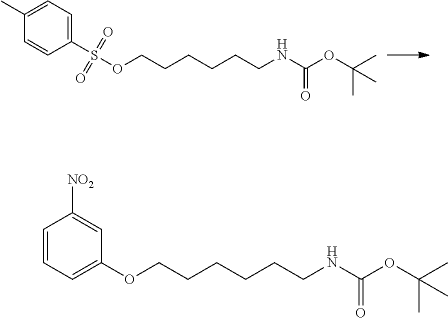 [SMILES: Cc1ccc(S(=O)(=O)OCCCCCCNC(=O)OC(C)(C)C)cc1 ; CC(C)(C)OC(=O)NCCCCCCOc1cccc([N+](=O)[O-])c1]

Prepared according to General Procedure E 6-((tert-butoxycarbonyl)amino)hexyl 4-methylbenzenesulfonate (701.8 mg, 1 Eq, 1.889 mmol), DMF (5 mL), 3-nitrophenol (341.6 mg, 1.3 Eq, 2.456 mmol) and potassium carbonate (391.6 mg, 1.5 Eq, 2.834 mmol). Normal phase chromatography over silica gel (0-25% EtOAc in hexanes) provided to give tert-butyl (6-(3-nitrophenoxy)hexyl)carbamate (326.4 mg, 964.5 μmol, 51.06%) as a clear oil. ¹H NMR (400 MHz, cdcl₃) δ 7.80 (dd, J=8.1, 1.3 Hz, 1H), 7.71 (t, J=2.3 Hz, 1H), 7.41 (t, J=8.2 Hz, 1H), 7.20 (dd, J=8.0, 2.1 Hz, 1H), 4.52 (s, 1H), 4.02 (t, J=6.4 Hz, 2H), 3.23-3.01 (m, 2H), 1.82 (dt, J=14.3, 6.5 Hz, 2H), 1.51 (dq, J=15.1, 7.4 Hz, 4H), 1.44 (s, 9H), 1.42-1.34 (m, 2H)

### Example 49

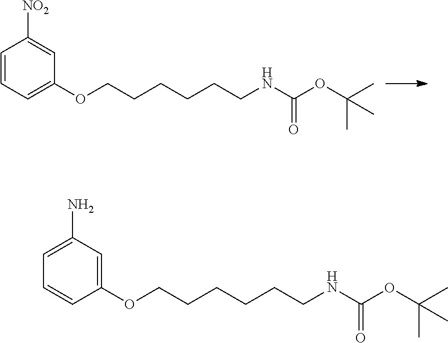 [SMILES: CC(C)(C)OC(=O)NCCCCCCOc1cccc([N+](=O)[O-])c1 ; CC(C)(C)OC(=O)NCCCCCCOc1cccc(N)c1]

Prepared according to General Procedure C using tert-butyl (6-(3-nitrophenoxy)hexyl)carbamate (326.4 mg, 1 Eq, 964.5 μmol), ethyl acetate (10 mL) and Pd/C (41.06 mg, 10% Wt, 0.04 Eq, 38.58 μmol) to give tert-butyl (6-(3-aminophenoxy)hexyl) carbamate (290.2 mg, 940.9 μmol, 97.55%). The crude material was used in the next step without purification. ¹H NMR (400 MHz, cdcl₃) δ 6.99 (t, J=8.0 Hz, 1H), 6.34-6.21 (m, 2H), 6.20 (t, J=2.1 Hz, 1H), 3.85 (t, J=6.5 Hz, 2H), 3.75-3.47 (m, 2H), 3.19-2.86 (m, 2H), 1.71 (p, J=6.6 Hz, 2H), 1.54-1.44 (m, 2H), 1.42 (s, 9H), 1.37-1.26 (m, 2H). LCMS (ESI, +ve mode) expected m/z for [C₁₇H₂₉N₂O₃⁺] [M+H] 308.2, found 309.3.

### Example 50

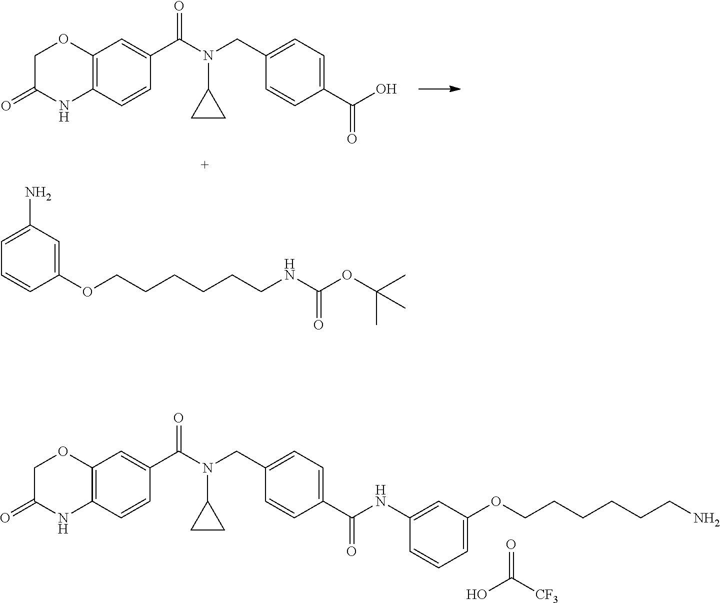

Prepared according to General Procedure A/Workup A using 4-((N-cyclopropyl-3-oxo-3,4-dihydro-2H-benzo[b][1,4]oxazine-7-carboxamido)methyl)benzoic acid (35 mg, 1 Eq, 95 μmol), EDC (27 mg, 1.5 Eq, 0.14 mmol), HOAt (19 mg, 1.5 Eq, 0.14 mmol), DMF (2 mL), tert-butyl (6-(3-aminophenoxy)hexyl)carbamate (50 mg, 1.7 Eq, 0.16 mmol) and triethylamine (39 mg, 53 μL, 4 Eq, 0.38 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((3-((6-aminohexyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (6.95 mg, 10.4 μmol, 11%) as a white solid. ¹H NMR (400 MHz, cd₃od) δ 7.91 (d, J=8.2 Hz, 2H), 7.52-7.29 (m, 3H), 7.26-7.17 (m, 2H), 7.17-7.03 (m, 2H), 6.93 (d, J=8.0 Hz, 1H), 6.77-6.50 (m, 1H), 4.76 (s, 2H), 4.57 (s, 2H), 3.95 (t, J=6.3 Hz, 2H), 2.91 (t, J=7.6 Hz, 2H), 2.75 (s, 1H), 1.76 (p, J=6.4 Hz, 2H), 1.66 (p, J=7.6 Hz, 2H), 1.57-1.30 (m, 4H), 0.76-0.55 (m, 2H), 0.55-0.24 (m, 2H). LCMS (ESI, +ve mode) expected m/z for [C₃₂H₃₇N₄O₅⁺] [M+H] 557.3, found 557.3.

### Example 51

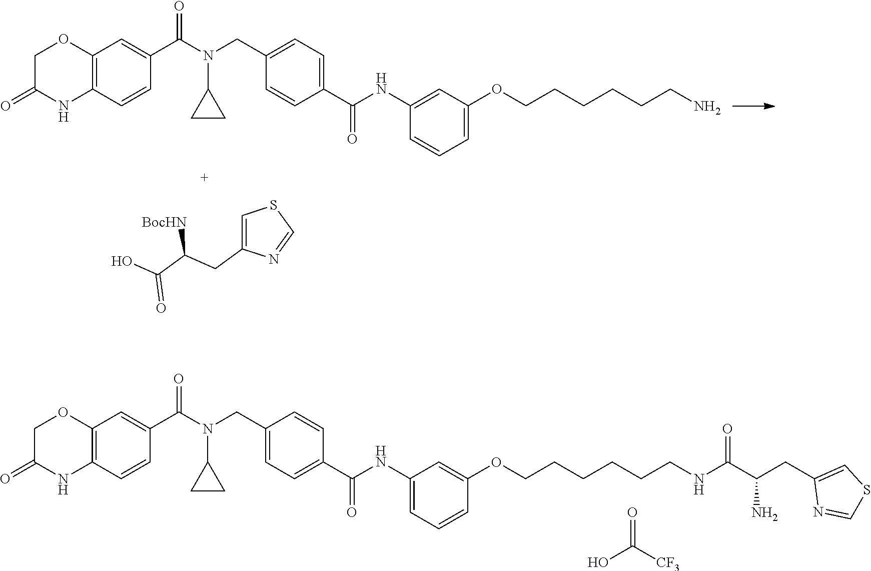

Prepared according to General Procedure B/Workup A using Boc-3-(4-Thiazolyl)-L-alanine (8.9 mg, 1.1 Eq, 33 μmol), TBTU (12 mg, 1.3 Eq, 39 μmol), DMF (1 mL), N-(4-((3-((6-aminohexyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 30 μmol) and DIPEA (13 mg, 17 μL, 3.3 Eq, 98 μmol). Following N-Boc deprotection, the product was purified by reverse phase chromatography (10-100% methanol in water+0.10% TFA) to give (S)—N-(4-((3-((6-(2-amino-3-(thiazol-4-yl)propanamido)hexyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (4.4 mg, 5.3 μmol, 18%) as a white solid. ¹H NMR (400 MHz, cd₃od) δ9.00 (d, J=1.9 Hz, 1H), 7.93 (d, J=8.2 Hz, 2H), 7.59-7.42 (m, 3H), 7.40 (d, J=1.8 Hz, 1H), 7.29-7.07 (m, 4H), 6.96 (d, J=8.1 Hz, 1H), 6.79-6.63 (m, 1H), 4.83 (s, 2H), 4.62 (s, 2H), 4.19 (dd, J=7.5, 5.9 Hz, 1H), 4.00 (t, J=6.3 Hz, 2H), 3.41-3.32 (m, 2H), 3.29-3.21 (m, 1H), 3.14 (dt, J=13.5, 6.8 Hz, 1H), 2.83 (s, 1H), 1.79 (p, J=6.5 Hz, 2H), 1.60-1.42 (m, 4H), 1.42-1.24 (m, 2H), 0.85-0.62 (m, 2H), 0.62-0.29 (m, 2H). LCMS (ESI, +ve mode) expected m/z for [C₃₈H₄₃N₆O₆S⁺] [M+H] 711.3, found 711.2.

### Example 52

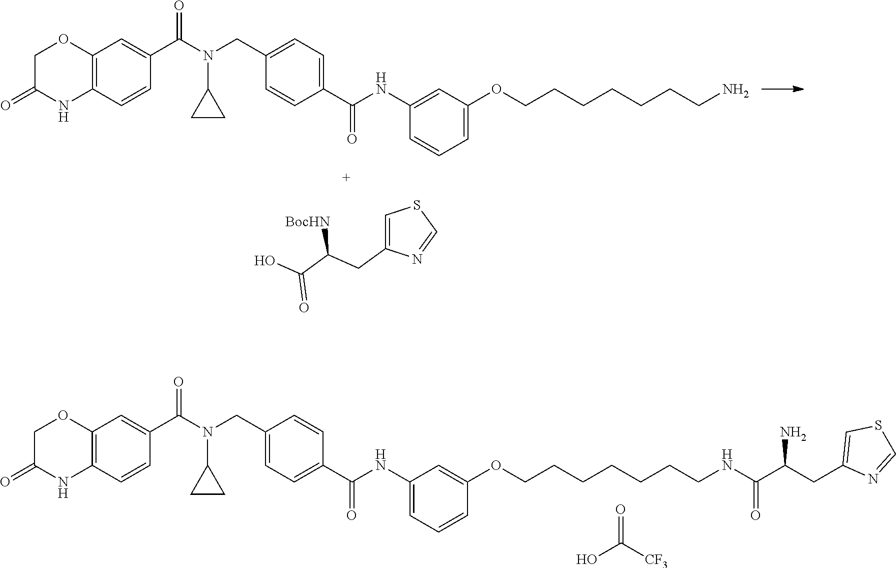

Prepared according to General Procedure B/Workup A using Boc-3-(4-Thiazolyl)-L-alanine (8.7 mg, 1.1 Eq, 32 μmol), TBTU (12 mg, 1.3 Eq, 38 μmol), DMF (1 mL), N-(4-((3-((7-aminoheptyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 29 μmol) and DIPEA (12 mg, 17 μL, 3.3 Eq, 96 μmol). Following N-Boc deprotection, the product was purified by reverse phase chromatography (10-100% methanol in water+0.1% TFA) to give (S)—N-(4-((3-((7-(2-amino-3-(thiazol-4-yl)propanamido)heptyl)oxy)phenyl)carbamoyl)benzyl)-N-cyclopropyl-3-oxo-3,4-dihydro-2H-benzo[b][1,4]oxazine-7-carboxamide 2,2,2-trifluoroacetate (2.48 mg, 2.96 μmol, 10%) as a white solid. ¹H NMR (400 MHz, cd₃od) δ 9.01 (d, J=1.9 Hz, 1H), 7.93 (d, J=8.2 Hz, 2H), 7.57-7.43 (m, 3H), 7.41 (d, J=1.9 Hz, 1H), 7.24 (t, J=8.2 Hz, 1H), 7.23-7.12 (m, 3H), 6.96 (d, J=8.1 Hz, 1H), 6.71 (d, J=8.1 Hz, 1H), 4.83 (s, 2H), 4.62 (s, 2H), 4.18 (dd, J=7.6, 5.7 Hz, 1H), 4.00 (t, J=6.4 Hz, 2H), 3.41-3.32 (m, 2H), 3.29-3.19 (m, 1H), 3.19-3.06 (m, 1H), 2.83 (s, 1H), 1.87-1.70 (m, 2H), 1.61-1.43 (m, 4H), 1.45-1.37 (m, 2H), 1.38-1.29 (m, 2H), 0.78-0.61 (m, 2H), 0.61-0.43 (m, 2H). LCMS (ESI, +ve mode) expected m/z for [C₃₉H₄₅N₆O₆S⁺] [M+H] 725.3, found 725.3.

### Example 53

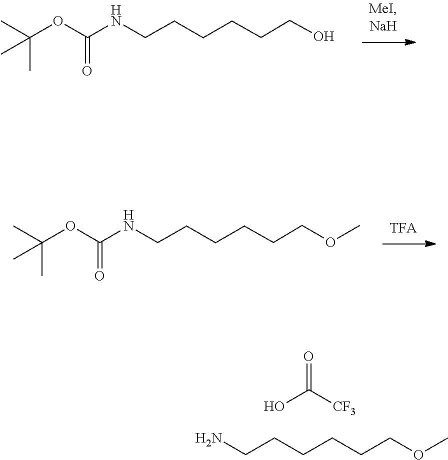

A solution of tert-butyl (6-hydroxyhexyl)carbamate (0.50 g, 1 Eq, 2.3 mmol) in DMF (5 mL) was cooled to 0° C. followed by the addition of sodium hydride (0.12 g, 60% Wt, 1.3 Eq, 3.0 mmol) and iodomethane (0.42 g, 0.19 mL, 1.3 Eq, 3.0 mmol). The reaction mixture was stirred at room temperature for 48 hours, after which the reaction mixture was quenched with H₂O (20 mL) and extracted 3× with EtOAc. The organic layer was washed with H₂O, brine (20 mL), and dried over MgSO4. Volatiles were removed under reduced pressure and normal phase chromatography over silica gel (0-25% EtOAc in hexanes) provided the N-Boc protected product as a colorless oil. Following N-Boc deprotection, solvent was removed to give 6-methoxyhexan-1-amine 2,2,2-trifluoroacetate (350.5 mg, 1.429 mmol, 62%), which was used without further purification. ¹H NMR (400 MHz, cdcl₃) δ 4.50 (s, 1H), 3.35 (t, J=6.5 Hz, 2H), 3.32 (s, 3H), 3.16-3.00 (m, 2H), 1.56 (p, J=6.5 Hz, 2H), 1.51-1.46 (m, 2H), 1.44 (s, 9H), 1.41-1.29 (m, 4H).

### Example 54

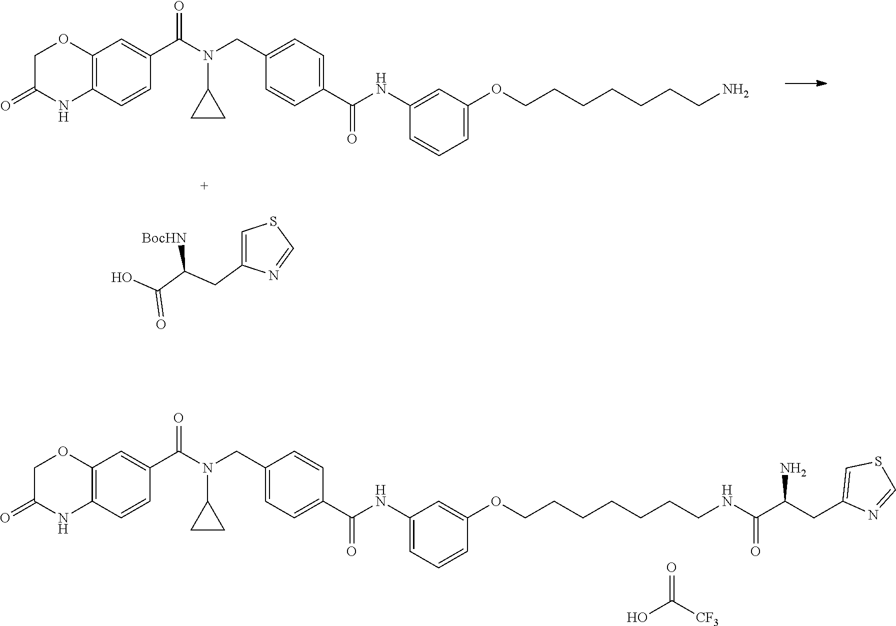 [SMILES: NCCCCCCCOc1cccc(NC(=O)c2ccc(CN(C(=O)c3ccc4c(c3)OCC(=O)N4)C3CC3)cc2)c1 ; CC(C)(C)OC(=O)N[C@@H](Cc1cscn1)C(=O)O ; N[C@@H](Cc1cscn1)C(=O)NCCCCCCCOc1cccc(NC(=O)c2ccc(CN(C(=O)c3ccc4c(c3)OCC(=O)N4)C3CC3)cc2)c1 ; O=C(O)C(F)(F)F]

Prepared and purified according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL) 6-methoxyhexan-1-amine 2,2,2-trifluoroacetate (25 mg, 1 Eq, 0.10 mmol) and triethylamine (42 mg, 57 μL, 4 Eq, 0.41 mmol). The product was purified by normal phase chromatography over silica gel (0-10% MeOH in DCM) to give N-cyclopropyl-N-(4-((4-((6-methoxyhexyl)carbamoyl)phenyl)carbamoyl)benzyl)-3-oxo-3,4-dihydro-2H-benzo[b][1,4]oxazine-7-carboxamide (2.64 mg, 4.41 μmol, 4.3%) as a white solid. ¹H NMR (400 MHz, cd₃od) δ 7.96 (d, J=8.3 Hz, 2H), 7.89-7.76 (m, 4H), 7.50 (d, J=7.8 Hz, 2H), 7.27-7.08 (m, 2H), 6.96 (d, J=8.0 Hz, 1H), 4.83 (s, 2H), 4.63 (s, 2H), 3.49-3.33 (m, 4H), 3.32 (s, 3H), 2.83 (s, 1H), 1.74-1.51 (m, 4H), 1.51-1.34 (m, 4H), 0.77-0.62 (m, 2H), 0.62-0.38 (m, 2H). LCMS (ESI, +ve mode) expected m/z for [C₃₄H₃₉N₄O₆⁺] [M+H] 599.3, found 599.2.

### Example 55

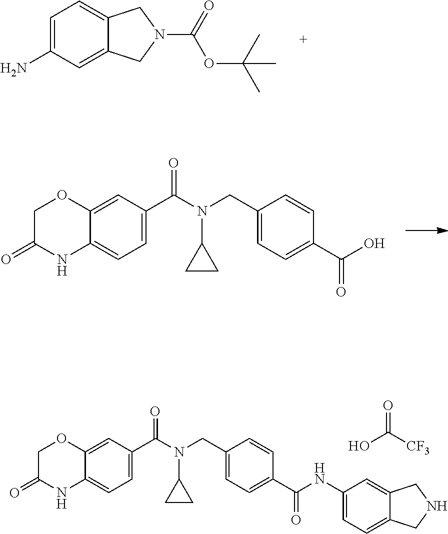

Prepared according to General Procedure A/Workup A using 4-((N-cyclopropyl-3-oxo-3,4-dihydro-2H-benzo[b][1,4]oxazine-7-carboxamido)methyl)benzoic acid (100 mg, 1 Eq, 273 μmol), EDC (78.5 mg, 1.5 Eq, 409 μmol), HOAt (55.7 mg, 1.5 Eq, 409 μmol), and DMF (2 mL), tert-Butyl 5-aminoisoindoline-2-carboxylate (76.7 mg, 1.2 Eq, 328 μmol) and triethylamine (82.9 mg, 114 μL, 3 Eq, 819 μmol). Following N-Boc deprotection, the product was purified by reverse phase chromatography (10-100% methanol in water+0.1% TFA) to give N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (53.3 mg, 89.3 μmol, 32.7%). ¹H NMR (400 MHz, Methanol-d₄) δ 7.96-7.89 (m, 2H), 7.87 (d, J=1.9 Hz, 1H), 7.65 (dd, J=8.3, 2.0 Hz, 1H), 7.51-7.39 (m, 2H), 7.36 (d, J=8.4 Hz, 1H), 7.17 (dd, J=8.1, 1.7 Hz, 1H), 7.13 (d, J=1.7 Hz, 1H), 6.94 (d, J=8.1 Hz, 1H), 4.79 (s, 2H), 4.61 (s, 2H), 4.59 (s, 2H), 4.58 (s, 2H), 2.87-2.72 (m, 1H), 0.69-0.57 (m, 2H), 0.57-0.43 (m, 2H). ¹³C NMR (101 MHz, cd₃od) δ173.87, 168.56, 168.47, 167.37, 167.27, 162.25, 161.88, 144.50, 143.46, 140.67, 136.34, 134.94, 133.21, 131.19, 129.79, 129.10, 128.78, 124.24, 123.16, 122.64, 116.83, 116.73, 116.39, 68.05, 51.89, 51.63, 33.70, 10.54. LCMS (ESI, +ve mode) expected m/z for [C₂₈H₂₇N₄O₄⁺] [M+H] 483.2, found 483.2.

### Example 56

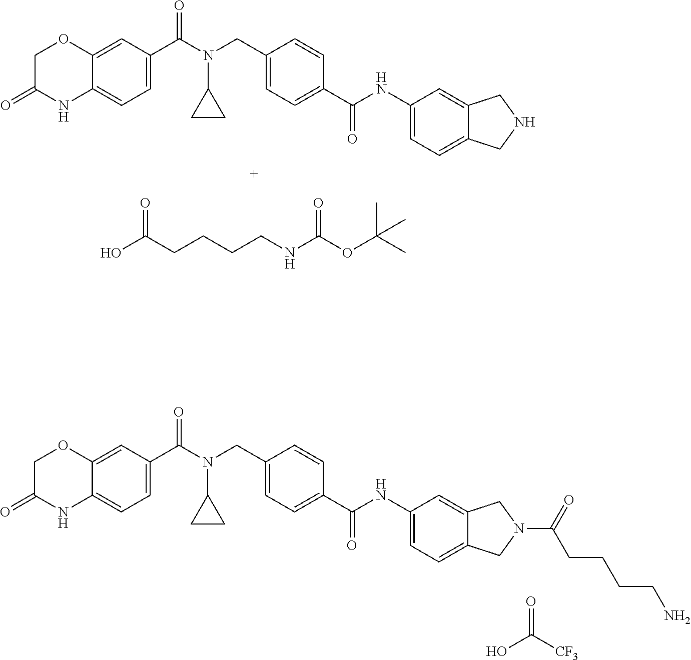 [SMILES: O=C1COc2cc(C(=O)N(Cc3ccc(C(=O)Nc4ccc5c(c4)CNC5)cc3)C3CC3)ccc2N1 ; CC(C)(C)OC(=O)NCCCCC(=O)O ; NCCCCC(=O)N1Cc2ccc(NC(=O)c3ccc(CN(C(=O)c4ccc5c(c4)OCC(=O)N5)C4CC4)cc3)cc2C1 ; O=C(O)C(F)(F)F]

Prepared according to General Procedure A/Workup A using 5-((tert-butoxycarbonyl)amino)pentanoic acid (18 mg, 1 Eq, 84 μmol), EDC (32 mg, 2 Eq, 0.17 mmol), HOAt (23 mg, 2 Eq, 0.17 mmol), DMF (1.5 mL), N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (50 mg, 1 Eq, 84 μmol) and triethylamine (34 mg, 47 μL, 4 Eq, 0.34 mmol). Following N-Boc deprotection, the product was purified by reverse phase chromatography (10-100% methanol in water+0.1% TFA) to give N-(4-((2-(5-aminopentanoyl)isoindolin-5-yl)carbamoyl)benzyl)-N-cyclopropyl-3-oxo-3,4-dihydro-2H-benzo[b][1,4]oxazine-7-carboxamide 2,2,2-trifluoroacetate (50.53 mg, 72.63 μmol, 87%) as a white solid. ¹H NMR (400 MHz, cd₃od) δ 7.93 (d, J=8.2 Hz, 2H), 7.77 (d, J=26.1 Hz, 1H), 7.63-7.52 (m, 1H), 7.52-7.37 (m, 2H), 7.31 (dd, J=8.3, 4.2 Hz, 1H), 7.25-7.09 (m, 2H), 6.95 (d, J=8.0 Hz, 1H), 4.86 (d, J=10.1 Hz, 2H), 4.81 (s, 2H), 4.72 (d, J=9.0 Hz, 2H), 4.61 (s, 2H), 3.06-2.90 (m, 2H), 2.81 (s, 1H), 2.60-2.31 (m, 2H), 1.87-1.54 (m, 4H), 0.78-0.61 (m, 2H), 0.61-0.33 (m, 2H). LCMS (ESI, +ve mode) expected m/z for [C₃₃H₃₆N₅O₅⁺] [M+H] 582.3, found 582.2.

### Example 57

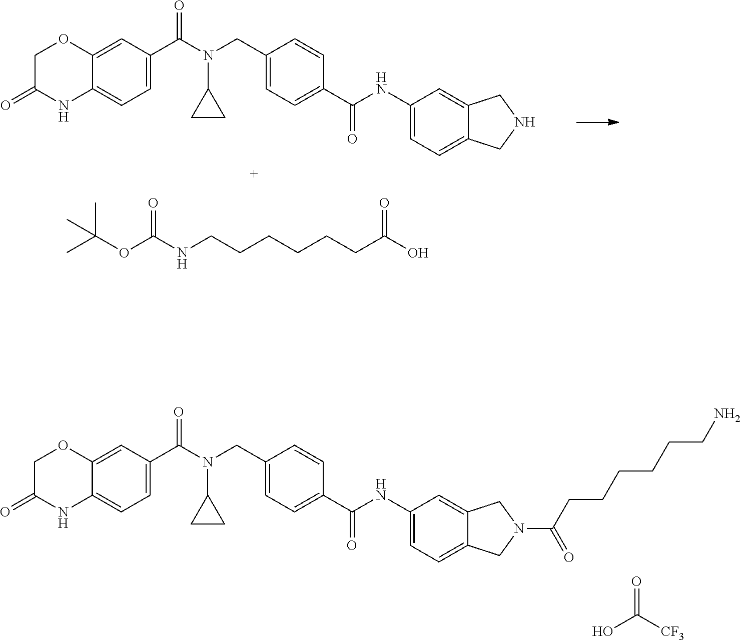 [SMILES: O=C1COc2cc(C(=O)N(Cc3ccc(C(=O)Nc4ccc5c(c4)CNC5)cc3)C3CC3)ccc2N1 ; CC(C)(C)OC(=O)NCCCCCCC(=O)O ; NCCCCCCC(=O)N1Cc2ccc(NC(=O)c3ccc(CN(C(=O)c4ccc5c(c4)OCC(=O)N5)C4CC4)cc3)cc2C1 ; O=C(O)C(F)(F)F]

Prepared according to General Procedure A/Workup A using 7-((tert-butoxycarbonyl)amino)heptanoic acid (7.16 mg, 1 Eq, 29.2 μmol), EDC (11.2 mg, 2 Eq, 58.3 μmol), HOAt (7.94 mg, 2 Eq, 58.3 μmol), DMF (0.5 mL), N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (17.4 mg, 1 Eq, 29.2 μmol) and triethylamine (11.8 mg, 16.3 μL, 4 Eq, 117 μmol). Following N-Boc deprotection, the product was purified by reverse phase chromatography (10-100% methanol in water+0.1% TFA) to give N-(4-((2-(7-aminoheptanoyl)isoindolin-5-yl)carbamoyl)benzyl)-N-cyclopropyl-3-oxo-3,4-dihydro-2H-benzo[b][1,4]oxazine-7-carboxamide 2,2,2-trifluoroacetate (7.99 mg, 11.0 μmol, 37.8%) as a white solid. ¹H NMR (400 MHz, cd₃od) δ7.94 (d, J=8.2 Hz, 2H), 7.79 (d, J=36.9 Hz, 1H), 7.67-7.52 (m, 1H), 7.52-7.40 (m, 2H), 7.33 (dd, J=8.3, 4.8 Hz, 1H), 7.27-7.09 (m, 2H), 6.96 (d, J=8.0 Hz, 1H), 4.89 (d, J=13.7 Hz, 2H), 4.83 (s, 2H), 4.75 (d, J=10.9 Hz, 2H), 4.62 (s, 2H), 2.93 (t, J=7.6 Hz, 2H), 2.83 (s, 1H), 2.49 (t, J=6.7 Hz, 2H), 1.80-1.60 (m, 4H), 1.54-1.39 (m, 4H), 0.83-0.62 (m, 2H), 0.62-0.34 (m, 2H). LCMS (ESI, +ve mode) expected m/z for [C₃₅H₄₀N₅O₅⁺] [M+H] 610.3, found 610.3.

### Example 58

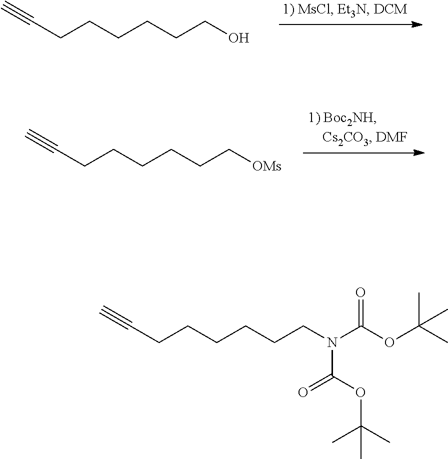

Prepared according to General Procedure F using (Step 1) oct-7-yn-1-ol (200 mg, 1 Eq, 1.58 mmol), DCM (5 mL), triethylamine (289 mg, 398 μL, 1.8 Eq, 2.85 mmol), mesyl chloride (254 mg 173 μL, 1.4 Eq, 2.22 mmol) and (Step 2) di-tert-butyl iminodicarbonate (413 mg, 1.2 Eq, 1.90 mmol), Cs₂CO₃ (775 mg, 1.5 Eq, 2.38 mmol) and DMF (2.5 mL). Normal phase chromatography over silica gel (0-25% EtOAc in hexanes) provided tert-butyl (tert-butoxycarbonyl)(oct-7-yn-1-yl)carbamate (400.3 mg, 1.230 mmol, 77.6%) as a clear oil. ¹H NMR (400 MHz, cdcl₃) δ 3.60-3.48 (m, 2H), 2.18 (td, J=7.0, 2.6 Hz, 2H), 1.93 (t, J=2.6 Hz, 1H), 1.70-1.52 (m, 4H), 1.50 (s, 18H), 1.46-1.38 (m, 2H), 1.35-1.23 (m, 2H). LCMS (ESI, +ve mode) expected m/z for [C₁₈H₃₁NO₄Na⁺] [M+Na] 348.2, found 348.3.

### Example 59

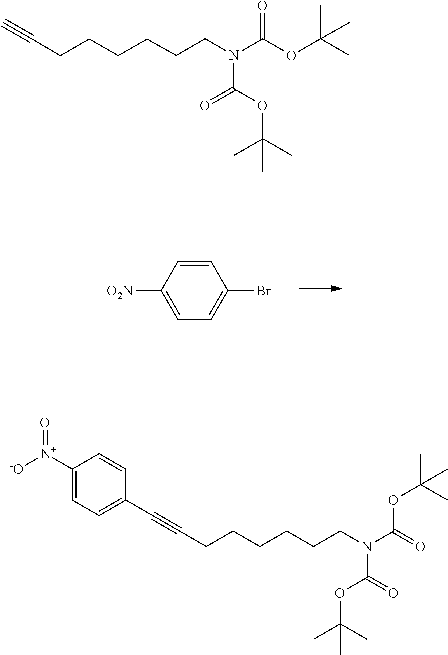

To a microwave vial charged with a stirbar was added di-tert-butyl oct-7-yn-1-yliminodicarbonate (400.2 mg, 1.5 Eq, 1.230 mmol), 1-bromo-4-nitrobenzene (0.1656 g, 1 Eq, 819.8 μmol), triphenylphosphine (21.50 mg, 0.1 Eq, 81.98 μmol), copper(I) iodide (15.61 mg, 0.1 Eq, 81.98 μmol), and Bis-(triphenylphosphino)-palladous chloride (28.77 mg, 0.05 Eq, 40.99 μmol). The atmosphere was evacuated and refilled with nitrogen three times before addition of anhydrous 1,4-Dioxane (4 mL). The solution was sparged with nitrogen for 15 minutes, then anhydrous triethylamine (290 mg, 400 μL, 3.50 Eq, 2.87 mmol) was added and a new vial cap which was sealed with parafilm. The reaction was heated to 80° C. overnight. The following day the reaction was cooled to room temperature and filtered through Celite, flushing with ethyl acetate. The filtrate was washed with saturated ammonium chloride, water, saturated sodium bicarbonate, brine, then then dried over MgSO₄, filtered and concentrated to a brown solid. Normal phase chromatography over silica gel (0-25% ethyl acetate in hexanes) provided di-tert-butyl (8-(4-nitrophenyl)oct-7-yn-1-yl)iminodicarbonate (359 mg, 804 μmol, 98.1%) as a yellow oil. ¹H NMR (400 MHz, cdcl₃) δ 8.15 (d, J=8.9 Hz, 2H), 7.51 (d, J=8.8 Hz, 2H), 3.64-3.44 (m, 2H), 2.44 (t, J=7.0 Hz, 2H), 1.71-1.52 (m, 6H), 1.50 (s, 18H), 1.38-1.30 (m, 2H).

### Example 60

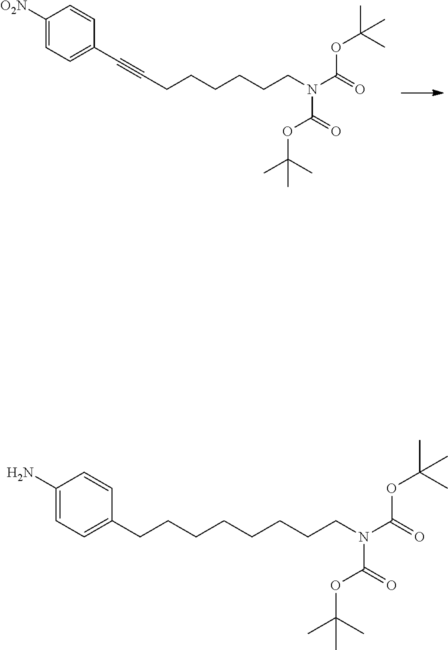 [SMILES: CC(C)(C)OC(=O)N(CCCCCCC#Cc1ccc([N+](=O)[O-])cc1)C(=O)OC(C)(C)C ; CC(C)(C)OC(=O)N(CCCCCCCCc1ccc(N)cc1)C(=O)OC(C)(C)C]

Prepared according to General Procedure C using tert-butyl (tert-butoxycarbonyl)(8-(4-nitrophenyl)oct-7-yn-1-yl)carbamate (376 mg, 1 Eq, 842 μmol), ethyl acetate (12 mL) and Pd/C (44.8 mg, 10% Wt, 0.05 Eq, 42.1 μmol). The crude material was purified by normal phase chromatography over silica gel (0-50% EtOAc in hexanes) to give tert-butyl (8-(4-aminophenyl)octyl)(tert-butoxycarbonyl)carbamate (253.2 mg, 602.0 μmol, 71.5%). ¹H NMR (400 MHz, cdcl₃) δ 6.94 (d, J=8.2 Hz, 2H), 6.60 (d, J=8.3 Hz, 2H), 3.55 (br s, 2H, —NH₂), 3.54-3.43 (m, 2H), 2.56-2.36 (m, 2H), 1.61-1.50 (m, 6H), 1.49 (s, 18H), 1.30-1.27 (m, 6H). LCMS (ESI, +ve mode) expected m/z for [C₂₄H₄₀N₂O₄Na⁺] [M+Na] 443.3, found 443.3.

### Example 61

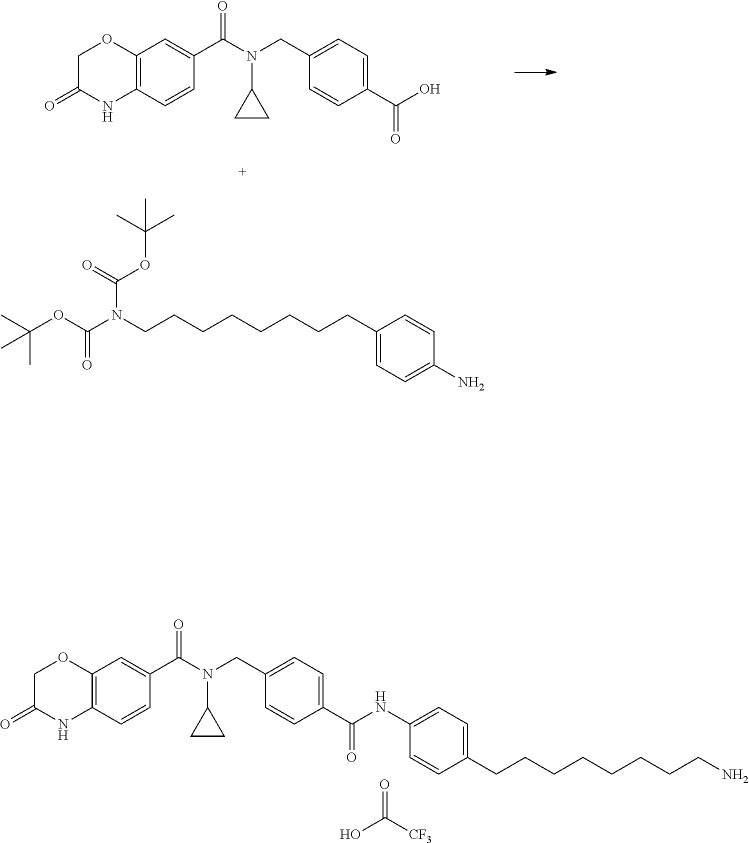

Prepared according to General Procedure A/Workup A using 4-((N-cyclopropyl-3-oxo-3,4-dihydro-2H-benzo[b][1,4]oxazine-7-carboxamido)methyl)benzoic acid (116.1 mg, 1 Eq, 316.8 μmol), EDC (91.11 mg, 1.5 Eq, 475.3 μmol), HOAt (64.69 mg, 1.5 Eq, 475.3 μmol), DMF (4 mL), tert-butyl (8-(4-aminophenyl)octyl)(tert-butoxycarbonyl)carbamate (253.2 mg, 1.9 Eq, 602.0 μmol) and triethylamine (128.2 mg, 0.18 mL, 4 Eq, 1.267 mmol). Following N-Boc deprotection, the product was purified by reverse phase chromatography (10-100% methanol in water+0.1% TFA). The product was then taken up in minimal methanol and triturated into ether to give N-(4-((4-(8-aminooctyl)phenyl)carbamoyl)benzyl)-N-cyclopropyl-3-oxo-3,4-dihydro-2H-benzo[b][1,4]oxazine-7-carboxamide 2,2,2-trifluoroacetate (73.65 mg, 107.9 μmol, 34.05%) as a white solid. ¹H NMR (400 MHz, cd₃od) δ 7.92 (d, J=8.1 Hz, 2H), 7.58 (d, J=8.3 Hz, 2H), 7.52-7.35 (m, 2H), 7.23-7.08 (m, 4H), 6.95 (d, J=8.0 Hz, 1H), 4.79 (s, 2H), 4.59 (s, 2H), 2.89 (t, J=7.6 Hz, 2H), 2.79 (s, 1H), 2.58 (t, J=7.5 Hz, 2H), 1.76-1.48 (m, 4H), 1.40-1.28 (m, 8H), 0.77-0.58 (m, 2H), 0.57-0.35 (m, 2H). LCMS (ESI, +ve mode) expected m/z for [C₃₄H₄₁N₄O₄⁺] [M+H] 569.7, found 569.3.

### Example 62

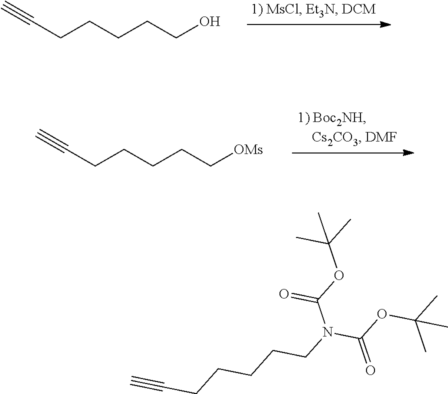

Prepared according to General Procedure F using (Step 1) hept-6-yn-1-ol (200 mg, 1 Eq, 1.78 mmol), DCM (5 mL), triethylamine (325 mg, 447 μL, 1.8 Eq, 3.21 mmol), mesyl chloride (286 mg 195 μL, 1.4 Eq, 2.50 mmol) and (Step 2) di-tert-butyl iminodicarbonate (465 mg, 1.2 Eq, 2.14 mmol), Cs2CO3 (871 mg, 1.5 Eq, 2.67 mmol), DMF (2.5 mL). Normal phase chromatography over silica gel (0-25% EtOAc in hexanes) provided tert-butyl (tert-butoxycarbonyl)(hept-6-yn-1-yl)carbamate (390.6 mg, 1.254 mmol, 70.3%) as a clear oil. ¹H NMR (400 MHz, cdcl₃) δ 3.63-3.37 (m, 2H), 2.19 (td, J=7.0, 2.6 Hz, 2H), 1.92 (t, J=2.6 Hz, 1H), 1.63-1.53 (m, 4H), 1.50 (s, 18H), 1.46-1.34 (m, 2H). LCMS (ESI, +ve mode) expected m/z for [C₁₇H₂₉NO₄Na⁺] [M+Na] 334.2, found 334.2.

### Example 63

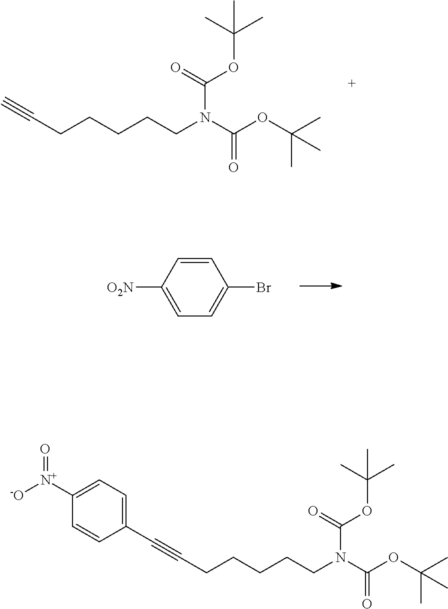

To a microwave vial charged with a stirbar was added di-tert-butyl hept-6-yn-1-yliminodicarbonate (391 mg, 1.5 Eq, 1.25 mmol), 1-bromo-4-nitrobenzene (0.169 g, 1 Eq, 837 μmol), triphenylphosphine (21.9 mg, 0.1 Eq, 83.7 μmol), copper(I)iodide (15.9 mg, 0.1 Eq, 83.7 μmol), and bis-(triphenylphosphino)-palladous chloride (29.4 mg, 0.05 Eq, 41.8 μmol). The atmosphere was evacuated and refilled with nitrogen three times before addition of anhydrous 1,4-Dioxane (4 mL). The solution was sparged with nitrogen for 15 minutes, then anhydrous triethylamine (290 mg, 400 μL, 3.43 Eq, 2.87 mmol) was added and a new vial cap which was sealed with parafilm. The reaction was heated to 80° C. overnight. The following day the reaction was cooled to room temperature and filtered through Celite, flushing with ethyl acetate. The filtrate was washed with saturated ammonium chloride, water, saturated sodium bicarbonate, brine, then then dried over MgSO₄, filtered and concentrated to a brown solid. Normal phase chromatography over silica gel (0-25% ethyl acetate in hexanes) provided di-tert-butyl (7-(4-nitrophenyl)hept-6-yn-1-yl)iminodicarbonate (359 mg, 830 μmol, 99.2%) as a yellow oil. ¹H NMR (400 MHz, cdcl₃) δ 8.15 (d, J=8.8 Hz, 2H), 7.51 (d, J=8.8 Hz, 2H), 3.63-3.56 (m, 2H), 2.45 (t, J=7.0 Hz, 2H), 1.71-1.57 (m, 4H), 1.50 (s, 18H), 1.48-1.39 (m, 2H).

### Example 64

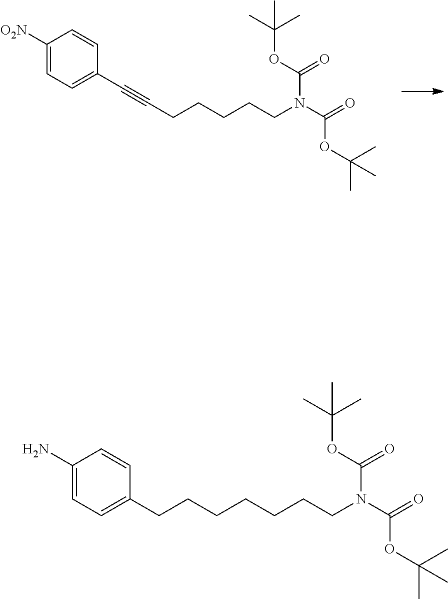 [SMILES: CC(C)(C)OC(=O)N(CCCCCC#Cc1ccc([N+](=O)[O-])cc1)C(=O)OC(C)(C)C ; CC(C)(C)OC(=O)N(CCCCCCCc1ccc(N)cc1)C(=O)OC(C)(C)C]

Prepared according to General Procedure C using tert-butyl (tert-butoxycarbonyl)(7-(4-nitrophenyl)hept-6-yn-1-yl)carbamate (371.2 mg, 1 Eq, 858.2 μmol), ethyl acetate (12 mL) and Pd/C (45.67 mg, 10% Wt, 0.05 Eq, 42.91 μmol). The crude material was purified by normal phase chromatography over silica gel (0-50% EtOAc in hexanes) to give tert-butyl (7-(4-aminophenyl)heptyl)(tert-butoxycarbonyl)carbamate (318.6 mg, 783.6 μmol, 91.31%). ¹H NMR (400 MHz, cdcl₃) δ 6.95 (d, J=8.2 Hz, 2H), 6.62 (d, J=8.3 Hz, 2H), 3.55 (br s, 2H, —NH₂), 3.54-3.49 (m, 2H), 2.61-2.25 (m, 2H), 1.59-1.52 (m, 4H), 1.50 (s, 18H), 1.34-1.24 (m, 6H).

### Example 65

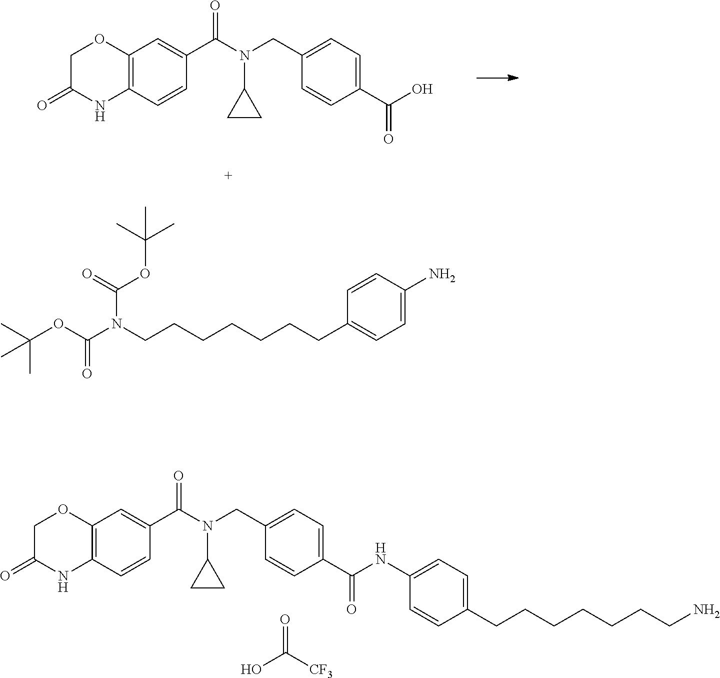

Prepared according to General Procedure A/Workup A using 4-((N-cyclopropyl-3-oxo-3,4-dihydro-2H-benzo[b][1,4]oxazine-7-carboxamido)methyl)benzoic acid (151.1 mg, 1 Eq, 412.4 μmol), EDC (118.6 mg, 1.5 Eq, 618.7 μmol), HOAt (84.21 mg, 1.5 Eq, 618.7 μmol), DMF (4 mL), tert-butyl (7-(4-aminophenyl)heptyl)(tert-butoxycarbonyl)carbamate (318.6 mg, 1.9 Eq, 783.6 μmol) and triethylamine (166.9 mg, 0.23 mL, 4 Eq, 1.650 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-(7-aminoheptyl)phenyl)carbamoyl)benzyl)-N-cyclopropyl-3-oxo-3,4-dihydro-2H-benzo[b][1,4]oxazine-7-carboxamide 2,2,2-trifluoroacetate (83.56 mg, 125.0 μmol, 30.30%) as a white solid. ¹H NMR (400 MHz, cd₃od) δ 7.92 (d, J=8.2 Hz, 2H), 7.58 (d, J=8.4 Hz, 2H), 7.52-7.33 (m, 2H), 7.27-7.07 (m, 4H), 6.95 (d, J=8.0 Hz, 1H), 4.79 (s, 2H), 4.59 (s, 2H), 2.89 (t, J=7.6 Hz, 2H), 2.79 (s, 1H), 2.59 (t, J=7.5 Hz, 2H), 1.73-1.51 (m, 4H), 1.45-1.21 (m, 6H), 0.83-0.58 (m, 2H), 0.58-0.29 (m, 2H). LCMS (ESI, +ve mode) expected m/z for [C₃₃H₃₉N₄O₄⁺] [M+H] 555.3, found 555.3.

### Example 66

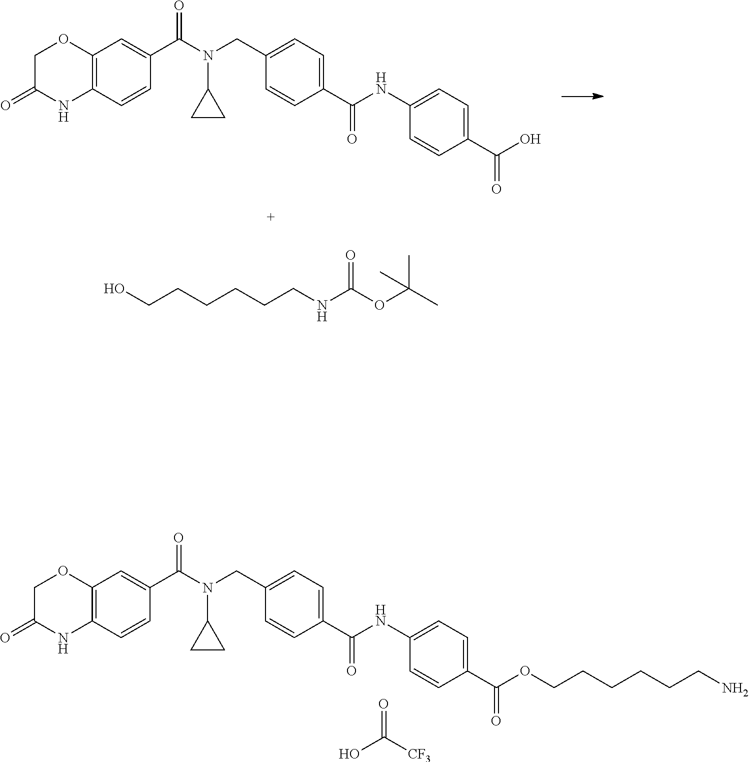

Prepared according to General Procedure G using 4-(4-((N-cyclopropyl-3-oxo-3,4-dihydro-2H-benzo[b][1,4]oxazine-7-carboxamido)methyl)benzamido)benzoic acid (70 mg, 1 Eq, 0.14 mmol), MeCN (5 mL), DMAP (1.8 mg, 0.1 Eq, 14 μmol), tert-butyl (6-hydroxyhexyl)carbamate (63 mg, 2 Eq, 0.29 mmol) and DCC (33 mg, 1.1 Eq, 0.16 mmol) at room temperature. The mixture was filtered to remove urea byproduct, and the filtrate was evaporated to dryness in vacuo yielding pale yellow residue. The resulting residue was dissolved in ethyl acetate, and washed with 1M citric acid, saturated NaHCO₃, and brine. The organic layer was dried over Na₂SO₄ and evaporated to give a white solid. Following N-Boc deprotection the crude material was purified by reverse phase chromatography (10-100% methanol in water+0.10% TFA) to give 6-aminohexyl 4-(4-((N-cyclopropyl-3-oxo-3,4-dihydro-2H-benzo[b][1,4]oxazine-7-carboxamido)methyl)benzamido) benzoate 2,2,2-trifluoroacetate (5.12 mg, 7.33 μmol, 5.1%) as a white solid. ¹H NMR (400 MHz, cd₃od) δ 8.01 (d, J=8.8 Hz, 2H), 7.95 (d, J=8.2 Hz, 2H), 7.86 (d, J=8.8 Hz, 2H), 7.50 (d, J=7.5 Hz, 2H), 7.25-7.10 (m, 2H), 6.96 (d, J=8.0 Hz, 1H), 4.83 (s, 2H), 4.62 (s, 2H), 4.34 (t, J=6.5 Hz, 2H), 2.93 (t, J=7.6 Hz, 2H), 2.83 (s, 1H), 1.82 (p, J=6.5 Hz, 2H), 1.69 (p, J=7.6 Hz, 2H), 1.60-1.36 (m, 4H), 0.82-0.61 (m, 2H), 0.61-0.37 (m, 2H). LCMS (ESI, +ve mode) expected m/z for [C₃₃H₃₇N₄O₆⁺] [M+H] 585.3, found 585.2.

### Example 67

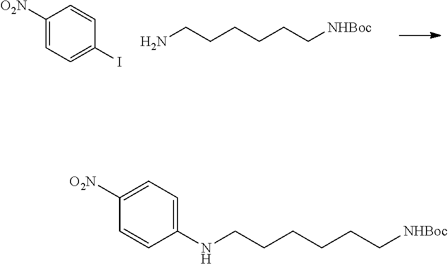 [SMILES: O=[N+]([O-])c1ccc(I)cc1 ; CC(C)(C)OC(=O)NCCCCCCN ; CC(C)(C)OC(=O)NCCCCCCNc1ccc([N+](=O)[O-])cc1]

An oven-dried microwave tube equipped with a Teflon valve was charged with a magnetic stir bar, copper(I)iodide (9.5 mg, 0.050 Eq, 0.050 mmol), cesium carbonate (652 mg, 2.00 Eq, 2.00 mmol), and 1-iodo-4-nitrobenzene (249 mg, 1 Eq, 1.00 mmol). The tube was evacuated and backfilled with nitrogen three times. Under a counter flow of nitrogen, DMF (1 mL) was added by syringe. Finally, 2-acetylcyclohexan-1-one (28 mg, 26 μL, 0.20 Eq, 0.20 mmol) then tert-butyl (6-aminohexyl)carbamate (0.32 g, 1.5 Eq, 1.5 mmol) were added via syringe. The vial was uncapped, flushed with nitrogen and recapped with a fresh cap under positive flow of nitrogen. The dark, olive green mixture was allowed to stir under argon at ambient temperature overnight. Upon completion of the reaction, the mixture was diluted with ethyl acetate, passed through a fritted glass filter to remove the inorganic salts, and concentrated in vacuo. The dark green residue was then diluted with water and extracted three times with ethyl acetate. The combined organics were washed twice with saturated NaHCO₃, once with water, once with brine, dried over magnesium sulfate, filtered and concentrated in vacuo. The now dark red residue was purified by column chromatography on silica gel (0-100% EtOAc in hexanes) to give tert-butyl (6-((4-nitrophenyl)amino)hexyl)carbamate (191.9 mg, 568.7 μmol, 56.9%) as a dark orange oil. ¹H NMR (400 MHz, cdcl₃) δ 8.03 (d, J=9.1 Hz, 2H), 6.49 (d, J=9.2 Hz, 2H), 4.89 (br s, 1H, NH), 4.60 (br s, 1H, NH), 3.17 (q, J=6.7 Hz, 2H), 3.09 (q, J=6.2 Hz, 2H), 1.62 (p, J=7.0 Hz, 2H), 1.54-1.43 (m, 2H), 1.41 (s, 9H), 1.40-1.28 (m, 4H). LCMS (ESI, +ve mode) expected m/z for [C₁₇H₂₇N₃O₄Na⁺] [M+Na] 360.2, found 360.2.

### Example 68

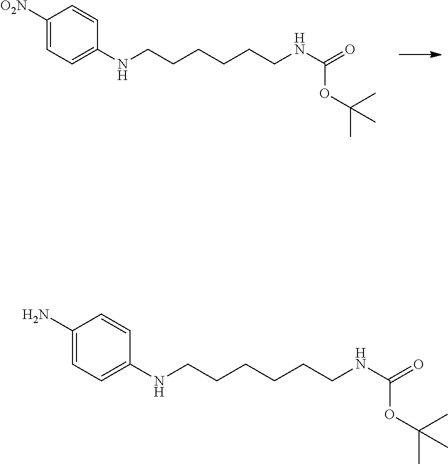 [SMILES: CC(C)(C)OC(=O)NCCCCCCNc1ccc([N+](=O)[O-])cc1 ; CC(C)(C)OC(=O)NCCCCCCNc1ccc(N)cc1]

Prepared according to General Procedure C using tert-butyl (6-((4-nitrophenyl)amino)hexyl)carbamate (191.9 mg, 1 Eq, 568.7 μmol), ethyl acetate (5 mL) and Pd/C (30.26 mg, 10% Wt, 0.05 Eq, 28.44 μmol) to give a deep ruby oil. The crude material was purified by normal phase chromatography over silica gel (0-10% MeOH in DCM) to give tert-butyl (6-((4-aminophenyl)amino)hexyl)carbamate (90.6 mg, 295 μmol, 51.8%) as a darkbrown oil. ¹H NMR (400 MHz, cdcl₃) δ 6.59 (d, J=8.6 Hz, 2H), 6.49 (d, J=8.6 Hz, 2H), 4.57 (br s, 1H, NH), 3.21 (br s, 3H, NH, NH₂), 3.09 (q, J=6.3 Hz, 3H), 3.02 (t, J=7.1 Hz, 2H), 1.57 (p, J=7.1 Hz, 2H), 1.52-1.45 (m, 2H), 1.43 (s, 9H), 1.39-1.27 (m, 4H).

### Example 69

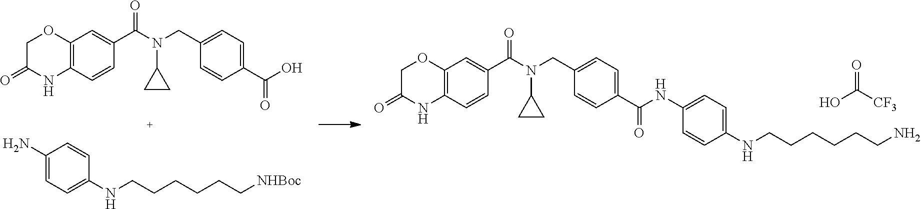 [SMILES: O=C1COc2cc(C(=O)N(Cc3ccc(C(=O)O)cc3)C3CC3)ccc2N1 ; CC(C)(C)OC(=O)NCCCCCCNc1ccc(N)cc1 ; NCCCCCCNc1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (56.8 mg, 1 Eq, 155 μmol), EDC (44.6 mg, 1.5 Eq, 233 μmol), HOAt (31.7 mg, 1.5 Eq, 233 μmol), DMF (2 mL), tert-butyl (6-((4-aminophenyl)amino)hexyl)carbamate (90.6 mg, 1.9 Eq, 295 μmol) and triethylamine (62.8 mg, 86 μL, 4 Eq, 620 μmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-((6-aminohexyl)amino)phenyl)carbamoyl)benzyl)-N-cyclopropyl-3-oxo-3,4-dihydro-2H-benzo[b][1,4]oxazine-7-carboxamide 2,2,2-trifluoroacetate (44.42 mg, 66.33 μmol, 42.8%) as a white solid. ¹H NMR (400 MHz, cd₃od) δ 8.06-7.84 (m, 4H), 7.61-7.38 (m, 4H), 7.28-7.05 (m, 2H), 6.96 (d, J=8.1 Hz, 1H), 4.82 (s, 2H), 4.61 (s, 2H), 3.49-3.36 (m, 2H), 2.97-2.88 (m, 2H), 2.82 (s, 1H), 1.77 (p, J=7.5 Hz, 2H), 1.67 (p, J=7.5 Hz, 2H), 1.54-1.32 (m, 4H), 0.82-0.60 (m, 2H), 0.60-0.36 (m, 2H). LCMS (ESI, +ve mode) expected m/z for [C₃₂H₃₈N₅O₄⁺] [M+H] 556.3, found 556.3.

### Example 70

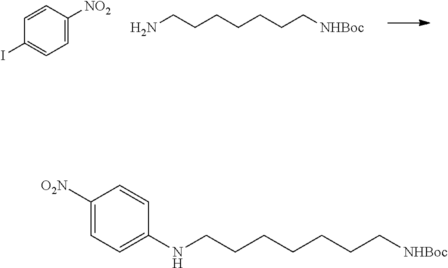 [SMILES: O=[N+]([O-])c1ccc(I)cc1 ; CC(C)(C)OC(=O)NCCCCCCCN ; CC(C)(C)OC(=O)NCCCCCCCNc1ccc([N+](=O)[O-])cc1]

An oven-dried microwave tube equipped with a Teflon valve was charged with a magnetic stir bar, copper(I)iodide (9.5 mg, 0.050 Eq, 0.050 mmol), cesium carbonate (652 mg, 2.00 Eq, 2.00 mmol), and 1-iodo-4-nitrobenzene (249 mg, 1 Eq, 1.00 mmol). The tube was evacuated and backfilled with three times. Under a counter flow of nitrogen DMF (1 mL) was added by syringe. Finally, tert-butyl (7-aminoheptyl)carbamate (230 mg, 1 Eq, 1.00 mmol) then 2-acetylcyclohexan-1-one (28 mg, 26 μL, 0.20 Eq, 0.20 mmol) were added via syringe. The vial was uncapped, flushed with nitrogen and recapped with a fresh cap under positive flow of nitrogen. The cap was sealed with parafilm. The dark, olive green mixture was allowed to stir under argon at ambient temperature overnight. Upon completion of the reaction, the mixture was diluted with ethyl acetate, passed through a fritted glass filter to remove the inorganic salts and the concentrated in vacuo. The dark green residue was then diluted with water and extracted three times with ethyl acetate. The combined organics were washed twice with saturated NaHCO₃, once with water, once with brine, dried over magnesium sulfate, filtered and concentrated in vacuo. The now dark red residue was purified by column chromatography on silica gel (0-100% EtOAc in hexanes) to give tert-butyl (7-((4-nitrophenyl)amino)heptyl)carbamate (315.1 mg, 896.6 μmol, 89.7%) as an orange solid. ¹H NMR (400 MHz, cdcl₃) δ 7.95 (d, J=9.1 Hz, 2H), 6.44 (d, J=9.1 Hz, 2H), 5.06 (br s, 1H), 4.71 (br s, 1H), 3.09 (q, J=6.7 Hz, 2H), 3.00 (q, J=6.4 Hz, 2H), 1.59-1.52 (m, 2H), 1.45-1.36 (m, 2H), 1.34 (s, 9H), 1.31-1.18 (m, 6H). LCMS (ESI, +ve mode) expected m/z for [C₁₈H₂₉N₃O₄Na⁺] [M+Na] 374.2, found 374.3.

### Example 71

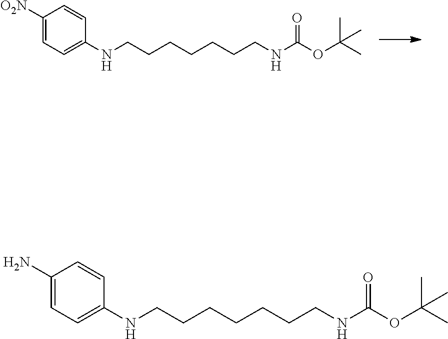 [SMILES: CC(C)(C)OC(=O)NCCCCCCCNc1ccc([N+](=O)[O-])cc1 ; CC(C)(C)OC(=O)NCCCCCCCNc1ccc(N)cc1]

Prepared according to General Procedure C using tert-butyl (7-((4-nitrophenyl)amino)heptyl)carbamate (315.1 mg, 1 Eq, 896.6 μmol), ethyl acetate (10 mL) and Pd/C (47.71 mg, 10% Wt, 0.05 Eq, 44.83 μmol). The crude material was purified by normal phase chromatography over silica gel (0-10% MeOH in DCM) to give tert-butyl (7-((4-aminophenyl)amino)heptyl)carbamate (132.8 mg, 413.1 μmol, 46.08%) as a light purple oil. ¹H NMR (400 MHz, cdcl₃) δ 6.59 (d, J=8.6 Hz, 2H), 6.49 (d, J=8.6 Hz, 2H), 4.58 (br s, 1H, NH), 3.23 (br s, 3H, NH, NH₂), 3.08 (q, J=6.2 Hz, 2H), 3.02 (t, J=7.1 Hz, 2H), 1.57 (p, J=7.0 Hz, 2H), 1.51-1.45 (m, 2H), 1.43 (s, 9H), 1.38-1.24 (m, 6H). LCMS (ESI, +ve mode) expected m/z for [C₁₈H₃₂N₃O₂][M+H] 322.2, found 322.3.

### Example 72

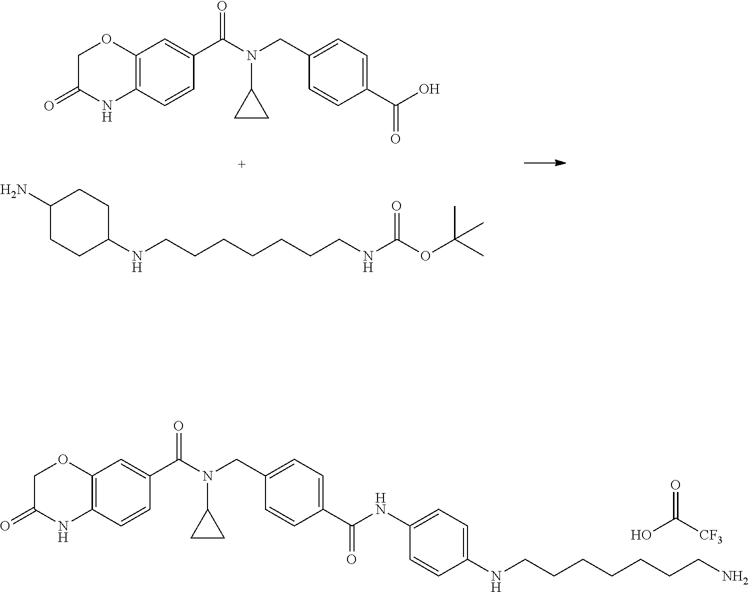

Prepared according to General Procedure A/Workup A using 4-((N-cyclopropyl-3-oxo-3,4-dihydro-2H-benzo[b][1,4]oxazine-7-carboxamido)methyl)benzoic acid (79.66 mg, 1 Eq, 217.4 μmol), EDC (62.52 mg, 1.5 Eq, 326.1 μmol), HOAt (44.39 mg, 1.5 Eq, 326.1 μmol), DMF (4 mL), tert-butyl (7-((4-aminophenyl)amino)heptyl)carbamate (132.8 mg, 1.9 Eq, 413.1 μmol) and triethylamine (88.00 mg, 0.12 mL, 4 Eq, 869.7 μmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-((7-aminoheptyl)amino)phenyl)carbamoyl)benzyl)-N-cyclopropyl-3-oxo-3,4-dihydro-2H-benzo[b][1,4]oxazine-7-carboxamide 2,2,2-trifluoroacetate (106.65 mg, 155.98 μmol, 71.74%) as an off-white solid. ¹H NMR (400 MHz, cd₃od) δ 8.04-7.84 (m, 4H), 7.57-7.40 (m, 4H), 7.26-7.10 (m, 2H), 6.95 (d, J=8.1 Hz, 1H), 4.81 (s, 2H), 4.60 (s, 2H), 3.44-3.33 (m, 2H), 2.96-2.86 (m, 2H), 2.82 (s, 1H), 1.83-1.69 (m, 2H), 1.69-1.57 (m, 2H), 1.50-1.27 (m, 6H), 0.79-0.59 (m, 2H), 0.59-0.34 (m, 2H). LCMS (ESI, +ve mode) expected m/z for [C₃₃H₄₀N₅O₄⁺] [M+H] 570.3, found 570.3.

### Example 73

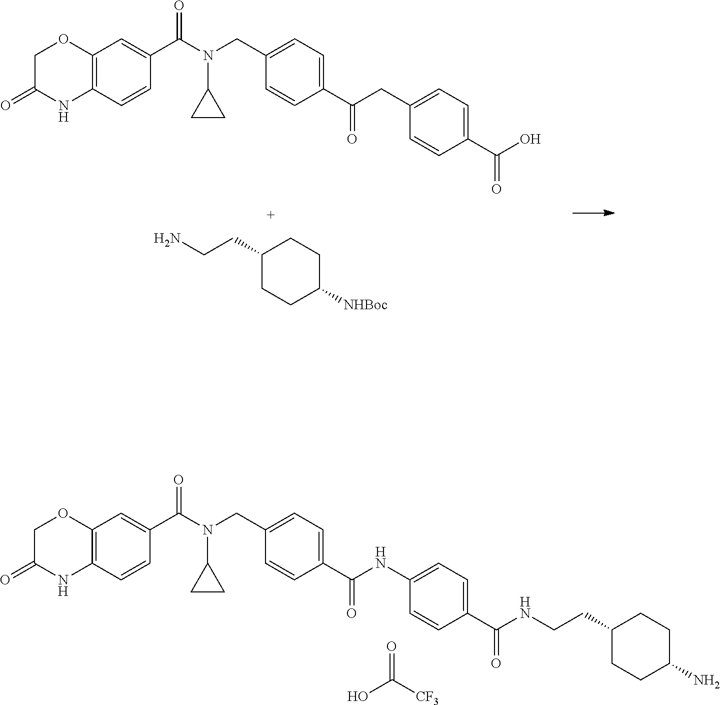

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), tert-butyl ((1s,4s)-4-(2-aminoethyl)cyclohexyl)carbamate (25 mg, 1 Eq, 0.10 mmol) and triethylamine (42 mg 57 μL, 4 Eq, 0.41 mmol). Following N-Boc deprotection, the product was purified by reverse phase chromatography (10-100% methanol in water+0.1% TFA) to give N-(4-((4-((2-((1s,4s)-4-aminocyclohexyl)ethyl)carbamoyl)phenyl)carbamoyl) benzyl)-N-cyclopropyl-3-oxo-3,4-dihydro-2H-benzo[b][1,4]oxazine-7-carboxamide 2,2,2-trifluoroacetate (11.26 mg, 15.56 μmol, 15%) as a white solid. ¹H NMR (400 MHz, cd₃od) δ 7.95 (d, J=8.3 Hz, 2H), 7.83 (s, 4H), 7.50 (d, J=7.8 Hz, 2H), 7.27-7.09 (m, 2H), 6.96 (d, J=8.0 Hz, 1H), 4.83 (s, 3H), 4.62 (s, 2H), 3.42 (t, J=6.7 Hz, 2H), 3.28 (s, 2H), 2.83 (s, 1H), 1.88-1.76 (m, 2H), 1.76-1.61 (m, 7H), 1.61-1.41 (m, 2H), 0.89-0.61 (m, 2H), 0.61-0.36 (m, 2H). LCMS (ESI, +ve mode) expected m/z for [C₃₅H₄₀N₅O₅⁺] [M+H] 610.3, found 610.3.

### Example 74

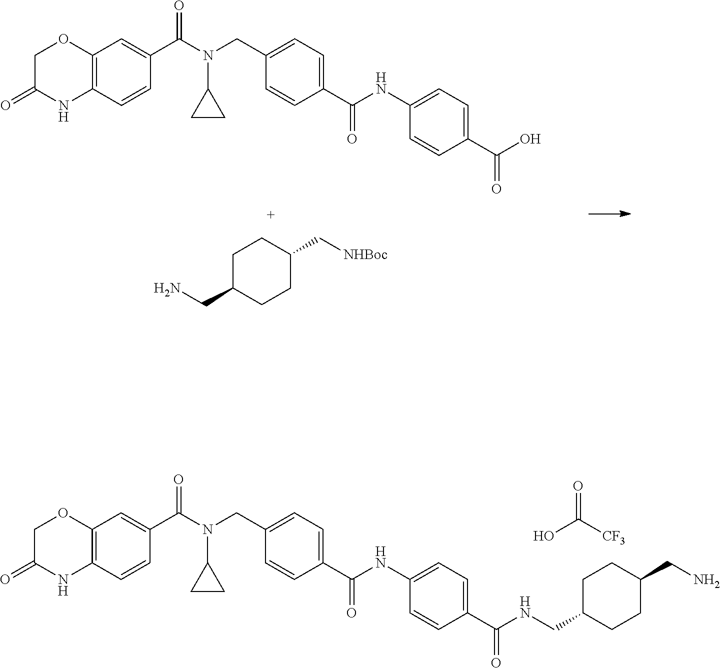

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), tert-butyl (((1r,4r)-4-(aminomethyl)cyclohexyl)methyl)carbamate (25 mg, 1 Eq, 0.10 mmol), and triethylamine (42 mg, 57 μL, 4 Eq, 0.41 mmol). Following N-Boc deprotection, the product was purified by reverse phase chromatography (10-100% methanol in water+0.1% TFA) to give N-(4-((4-((((1r,4r)-4-(aminomethyl)cyclohexyl)methyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (14.01 mg, 19.36 μmol, 19%) as white solid. ¹H NMR (400 MHz, cd₃od) δ 7.95 (d, J=8.3 Hz, 2H), 7.83 (s, 4H), 7.49 (d, J=7.7 Hz, 2H), 7.25-7.09 (m, 2H), 6.96 (d, J=8.0 Hz, 1H), 4.82 (s, 2H), 4.62 (s, 2H), 3.25 (d, J=6.9 Hz, 2H), 2.85 (s, 1H), 2.79 (d, J=7.0 Hz, 2H), 2.06-1.74 (m, 4H), 1.71-1.52 (m, 2H), 1.24-0.84 (m, 4H), 0.75-0.61 (m, 2H), 0.61-0.41 (m, 2H). LCMS(ESI, +ve mode) expected m/z for [C₃₅H₄₀N₅O₅+][M+H] 610.3, found 610.3.

### Example 75

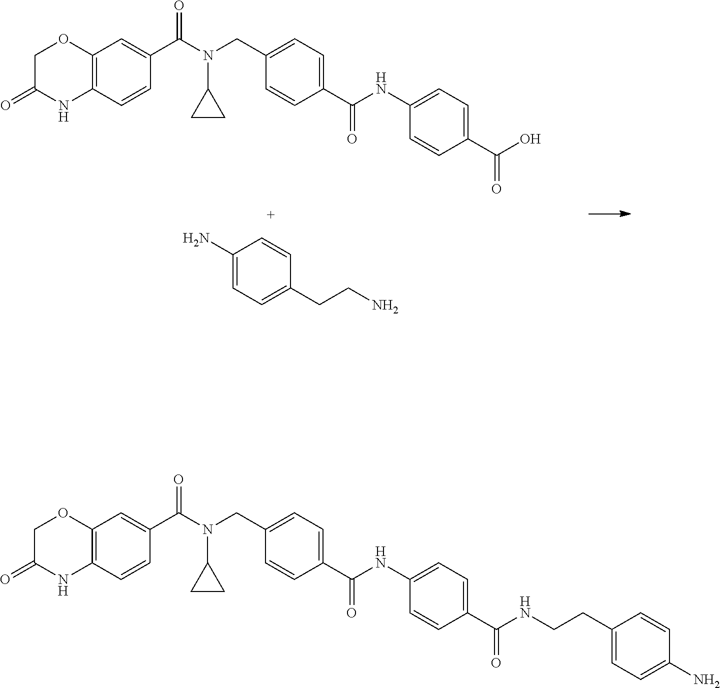 [SMILES: O=C1COc2cc(C(=O)N(Cc3ccc(C(=O)Nc4ccc(C(=O)O)cc4)cc3)C3CC3)ccc2N1 ; NCCc1ccc(N)cc1 ; Nc1ccc(CCNC(=O)c2ccc(NC(=O)c3ccc(CN(C(=O)c4ccc5c(c4)OCC(=O)N5)C4CC4)cc3)cc2)cc1]

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), 4-(2-aminoethyl) aniline (14 mg, 1 Eq, 0.10 mmol) and triethylamine (42 mg, 57 μL, 4 Eq, 0.41 mmol). The product was purified by reverse phase chromatography (10-100% methanol in water+0.1% TFA) to give N-(4-((4-((4-aminophenethyl)carbamoyl)phenyl)carbamoyl)benzyl)-N-cyclopropyl-3-oxo-3,4-dihydro-2H-benzo[b][1,4]oxazine-7-carboxamide (12.87 mg, 17.93 μmol, 17%) as a brown solid. ¹H NMR (400 MHz, cd₃od) δ 7.95 (d, J=8.2 Hz, 2H), 7.80 (q, J=8.9 Hz, 4H), 7.50 (d, J=7.7 Hz, 2H), 7.44 (d, J=8.4 Hz, 2H), 7.30 (d, J=8.4 Hz, 2H), 7.25-7.09 (m, 2H), 6.96 (d, J=8.0 Hz, 1H), 4.83 (s, 14H), 4.62 (s, 2H), 3.63 (t, J=7.2 Hz, 2H), 2.98 (t, J=7.3 Hz, 2H), 2.83 (s, 1H), 0.83-0.62 (m, 2H), 0.61-0.33 (m, 2H). LCMS (ESI, +ve mode) expected m/z for [C₃₅H₃₄N₅O₅⁺] [M+H] 604.3, found 604.2.

### Example 76

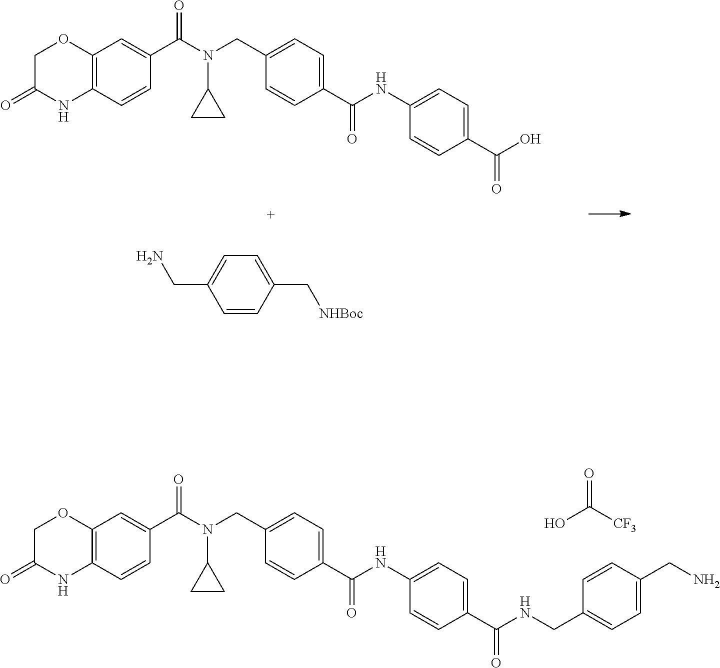

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), tert-butyl (4-(aminomethyl)benzyl)carbamate (24 mg, 1 Eq, 0.10 mmol) and triethylamine (42 mg, 57 μL, 4 Eq, 0.41 mmol). Following N-Boc deprotection, the product was purified by reverse phase chromatography (10-100% methanol in water+0.1% TFA) to give N-(4-((4-((4-(aminomethyl)benzyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (17.10 mg, 23.83 μmol, 23%) as a white solid. ¹H NMR (400 MHz, cd₃od) δ 7.95 (d, J=8.3 Hz, 2H), 7.91-7.78 (m, 4H), 7.49 (d, J=7.5 Hz, 2H), 7.47-7.30 (m, 4H), 7.28-7.00 (m, 2H), 6.96 (d, J=8.0 Hz, 1H), 4.82 (s, 2H), 4.61 (s, 2H), 4.59 (s, 2H), 4.09 (s, 2H), 2.82 (s, 1H), 0.84-0.60 (m, 2H), 0.60-0.33 (m, 2H). LCMS (ESI, +ve mode) expected m/z for [C₃₅H₃₄N₅O₅⁺] [M+H] 604.3, found 604.2.

### Example 77

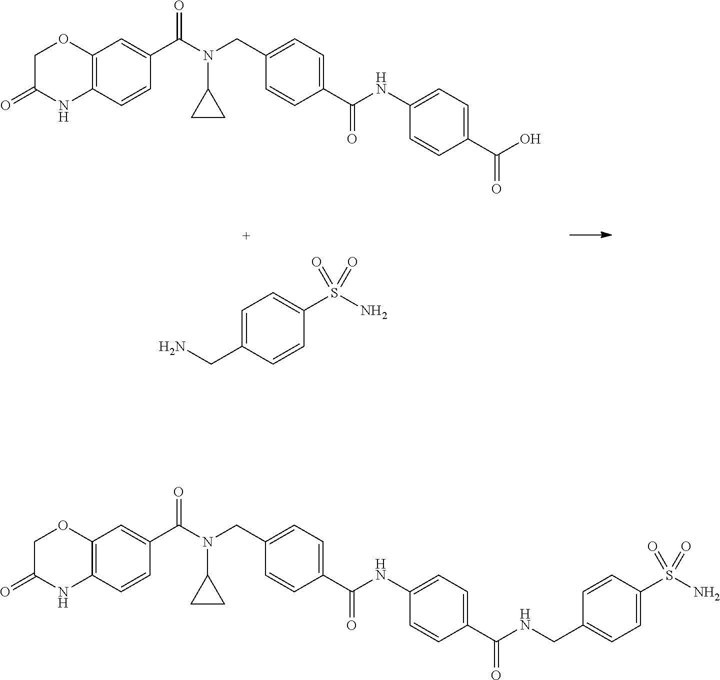 [SMILES: O=C1COc2cc(C(=O)N(Cc3ccc(C(=O)Nc4ccc(C(=O)O)cc4)cc3)C3CC3)ccc2N1 ; NCc1ccc(S(N)(=O)=O)cc1 ; NS(=O)(=O)c1ccc(CNC(=O)c2ccc(NC(=O)c3ccc(CN(C(=O)c4ccc5c(c4)OCC(=O)N5)C4CC4)cc3)cc2)cc1]

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), 4-(aminomethyl)benzenesulfonamide hydrochloride (23 mg, 1 Eq, 0.10 mmol) and triethylamine (42 mg, 57 μL, 4 Eq, 0.41 mmol). Chromatography could not be performed due to poor solubility. The product mixture was suspended in a methanol/water mixture and filtered to give N-cyclopropyl-3-oxo-N-(4-((4-((4-sulfamoylbenzyl)carbamoyl)phenyl)carbamoyl)benzyl)-3,4-dihydro-2H-benzo[b][1,4]oxazine-7-carboxamide (18.53 mg, 28.35 μmol, 28%) as a white solid. ¹H NMR (400 MHz, dmso) δ 10.43 (s, 1H, NH), 9.02 (s, 1H, NH), 7.94 (d, J=7.7 Hz, 2H), 7.92-7.80 (m, 4H), 7.76 (d, J=7.8 Hz, 2H), 7.58-7.36 (m, 4H), 7.28 (s, 2H, —SO₂NH₂), 7.23-7.02 (m, 2H), 6.91 (d, J=8.3 Hz, 1H), 4.70 (s, 2H), 4.60 (s, 2H), 4.56-4.40 (m, 2H), 2.78 (s, 1H), 0.65-0.50 (m, 2H), 0.50-0.28 (m, 2H).

### Example 78

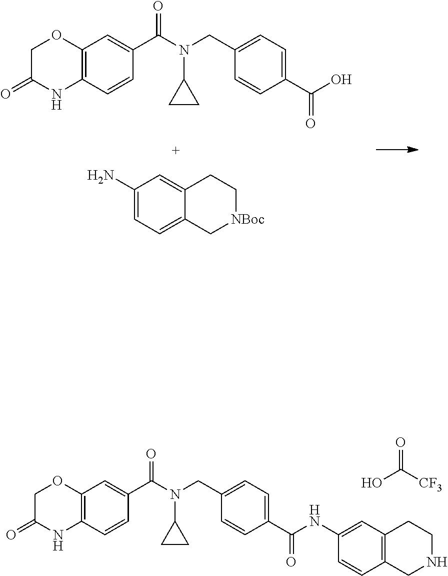 [SMILES: O=C1COc2cc(C(=O)N(Cc3ccc(C(=O)O)cc3)C3CC3)ccc2N1 ; CC(C)(C)OC(=O)N1CCc2cc(N)ccc2C1 ; O=C1COc2cc(C(=O)N(Cc3ccc(C(=O)Nc4ccc5c(c4)CCNC5)cc3)C3CC3)ccc2N1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (75 mg, 1 Eq, 0.20 mmol), EDC (59 mg, 1.5 Eq, 0.31 mmol), HOAt (42 mg, 1.5 Eq, 0.31 mmol), DMF (1.5 mL), tert-butyl 6-amino-3,4-dihydroisoquinoline-2(1H)-carboxylate (51 mg, 1 Eq, 0.20 mmol) and triethylamine (83 mg, 0.11 mL, 4 Eq, 0.82 mmol). Following N-Boc deprotection, the product was purified by reverse phase chromatography (10-100% methanol in water+0.1% TFA) to give N-cyclopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (105.4 mg, 172.6 μmol, 84%) as a clear resin. ¹H NMR (400 MHz, cd₃od) δ 7.92 (d, J=8.3 Hz, 2H), 7.66 (s, 1H), 7.58 (dd, J=8.4, 2.0 Hz, 1H), 7.46 (d, J=7.6 Hz, 2H), 7.28-7.06 (m, 3H), 6.95 (d, J=8.1 Hz, 1H), 4.80 (s, 2H), 4.60 (s, 2H), 4.33 (s, 2H), 3.49 (t, J=6.4 Hz, 2H), 3.11 (t, J=6.3 Hz, 2H), 2.81 (s, 1H), 0.93-0.58 (m, 2H), 0.58-0.24 (m, 2H). LCMS (ESI, +ve mode) expected m/z for [C₂₉H₂₉N₄O₄⁺] [M+H] 497.2, found 497.2.

### Example 79

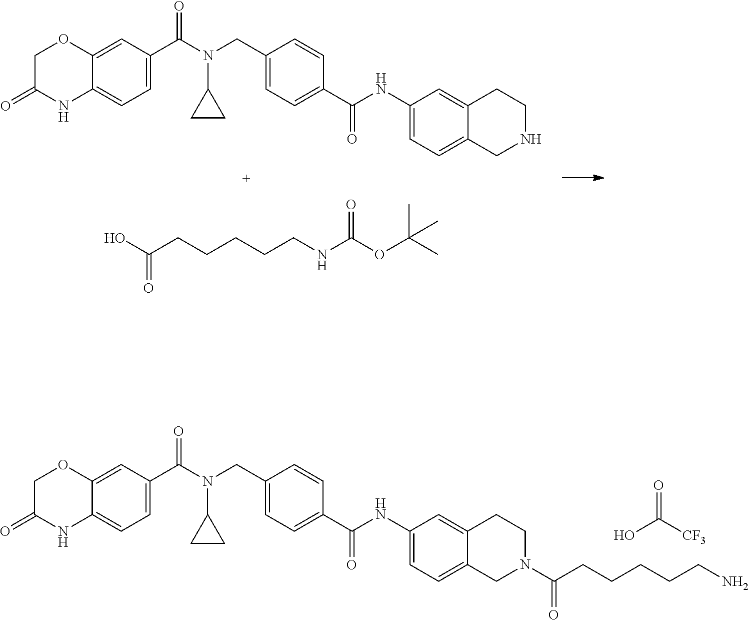

Prepared according to General Procedure A/Workup A using 6-((tert-butoxycarbonyl)amino)hexanoic acid (19 mg, 1 Eq, 82 μmol), EDC (31 mg, 2 Eq, 0.16 mmol), HOAt (22 mg, 2 Eq, 0.16 mmol), DMF (0.5 mL), N-cyclopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (50 mg, 1 Eq, 82 μmol) and triethylamine (33 mg, 46 μL, 4 Eq, 0.33 mmol). Following N-Boc deprotection, the product was purified by reverse phase chromatography (10-100% methanol in water+0.1% TFA) to give N-(4-((2-(6-aminohexanoyl)-1,2,3,4-tetrahydroisoquinolin-6-yl)carbamoyl)benzyl)-N-cyclopropyl-3-oxo-3,4-dihydro-2H-benzo[b][1,4]oxazine-7-carboxamide 2,2,2-trifluoroacetate (35.25 mg, 48.70 μmol, 59%) as a white solid. ¹H NMR (400 MHz, cd₃od) δ7.92 (d, J=8.2 Hz, 2H), 7.66-7.35 (m, 4H), 7.30-7.07 (m, 3H), 6.95 (d, J=8.1 Hz, 1H), 4.81 (s, 2H), 4.68 (d, J=5.6 Hz, 2H), 4.61 (s, 2H), 3.77 (dt, J=14.2, 6.0 Hz, 2H), 3.00-2.89 (m, 3H), 2.89-2.85 (m, 1H), 2.82 (s, 1H), 2.58-2.42 (m, 2H), 1.68 (h, J=7.8 Hz, 4H), 1.45 (hept, J=7.7 Hz, 2H), 0.80-0.60 (m, 2H), 0.61-0.33 (m, 2H). LCMS (ESI, +ve mode) expected m/z for [C₃₅H₄₀N₅O₅+][M+H] 610.3, found 610.3.

### Example 80

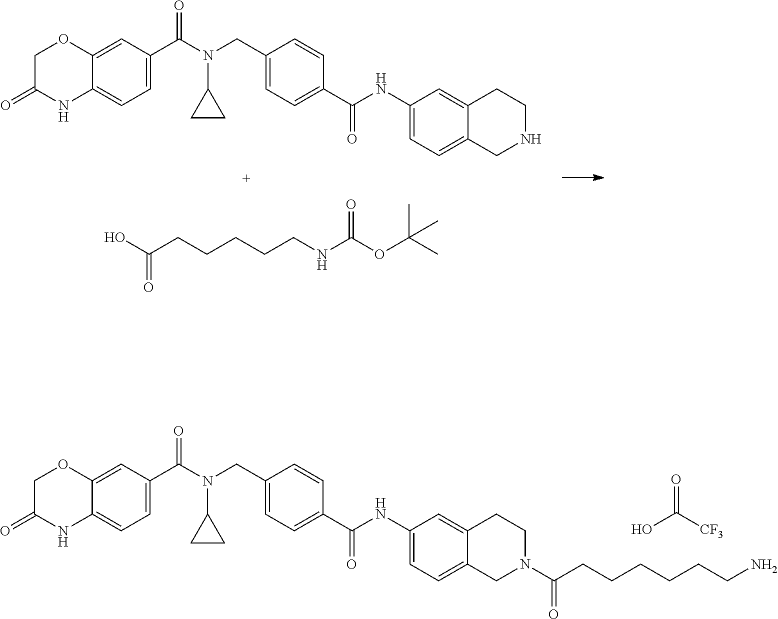

Prepared according to General Procedure A/Workup A using 7-((tert-butoxycarbonyl)amino)heptanoic acid (20 mg, 1 Eq, 82 μmol), EDC (31 mg, 2 Eq, 0.16 mmol), HOAt (22 mg, 2 Eq, 0.16 mmol), DMF (0.5 mL), N-cyclopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (50 mg, 1 Eq, 82 μmol) and triethylamine (33 mg, 46 μL, 4 Eq, 0.33 mmol). Following N-Boc deprotection, the product was purified by reverse phase chromatography (10-100% methanol in water+0.1% TFA) to give N-(4-((2-(7-aminoheptanoyl)-1,2,3,4-tetrahydroisoquinolin-6-yl)carbamoyl)benzyl)-N-cyclopropyl-3-oxo-3,4-dihydro-2H-benzo[b][1,4]oxazine-7-carboxamide 2,2,2-trifluoroacetate (30.40 mg, 41.20 μmol, 50%) as a white solid. ¹H NMR (400 MHz, cd₃od) δ7.92 (d, J=8.0 Hz, 2H), 7.66-7.31 (m, 4H), 7.27-7.05 (m, 3H), 6.95 (d, J=8.1 Hz, 1H), 4.81 (s, 2H), 4.68 (d, J=8.9 Hz, 2H), 4.61 (s, 2H), 3.77 (dt, J=12.2, 6.0 Hz, 2H), 2.98-2.87 (m, 3H), 2.87-2.84 (m, 1H), 2.82 (s, 1H), 2.50 (td, J=7.4, 4.5 Hz, 2H), 1.78-1.53 (m, 4H), 1.53-1.29 (m, 4H), 0.83-0.59 (m, 2H), 0.59-0.08 (m, 2H). LCMS (ESI, +ve mode) expected m/z for [C₃₆H₄₂N₅O₅+][M+H] 624.3, found 624.3.

### Example 81

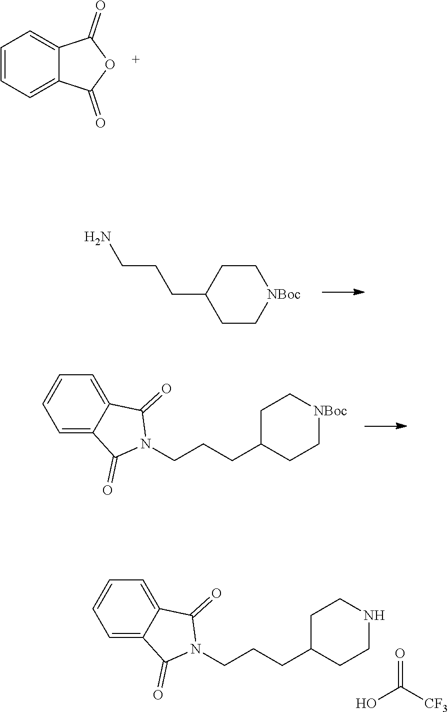

A flask containing isobenzofuran-1,3-dione (200 mg, 1 Eq, 1.35 mmol), tert-butyl 4-(3-aminopropyl)piperidine-1-carboxylate (327 mg, 1 Eq, 1.35 mmol), sodium acetate (133 mg, 1.2 Eq, 1.62 mmol), and acetic acid (8.11 g, 7.72 mL, 100 Eq, 135 mmol) was heated to 140° C. overnight. On cooling the reaction was diluted with water and washed twice with ethyl acetate. The combined organic layers were dried with sodium sulfate and concentrated in vacuo. Normal phase chromatography on silica gel (0-100% EtOAc in Hexanes) gave the di-protected intermediate. Following N-Boc deprotection, the product was purified by normal phase chromatography on silica gel (0-10% MeOH in DCM) to give 2-(3-(piperidin-4-yl)propyl)isoindoline-1,3-dione 2,2,2-trifluoroacetate (29.8 mg, 77.1 μmol, 5.71%) as a clear oil. ¹H NMR (400 MHz, cdcl₃) δ 7.82 (dd, J=5.4, 3.1 Hz, 2H), 7.70 (dd, J=5.5, 3.0 Hz, 2H), 3.66 (t, J=7.1 Hz, 2H), 3.36 (d, J=12.6 Hz, 2H), 2.82 (q, J=11.4 Hz, 2H), 1.85 (d, J=11.7 Hz, 2H), 1.68 (p, J=7.7 Hz, 2H), 1.61-1.38 (m, 3H), 1.38-1.29 (m, 2H). LCMS (ESI, +ve mode) expected m/z for [C₁₆H₂₁N₂O₂⁺] [M+H] 273.2, found 273.2.

### Example 82

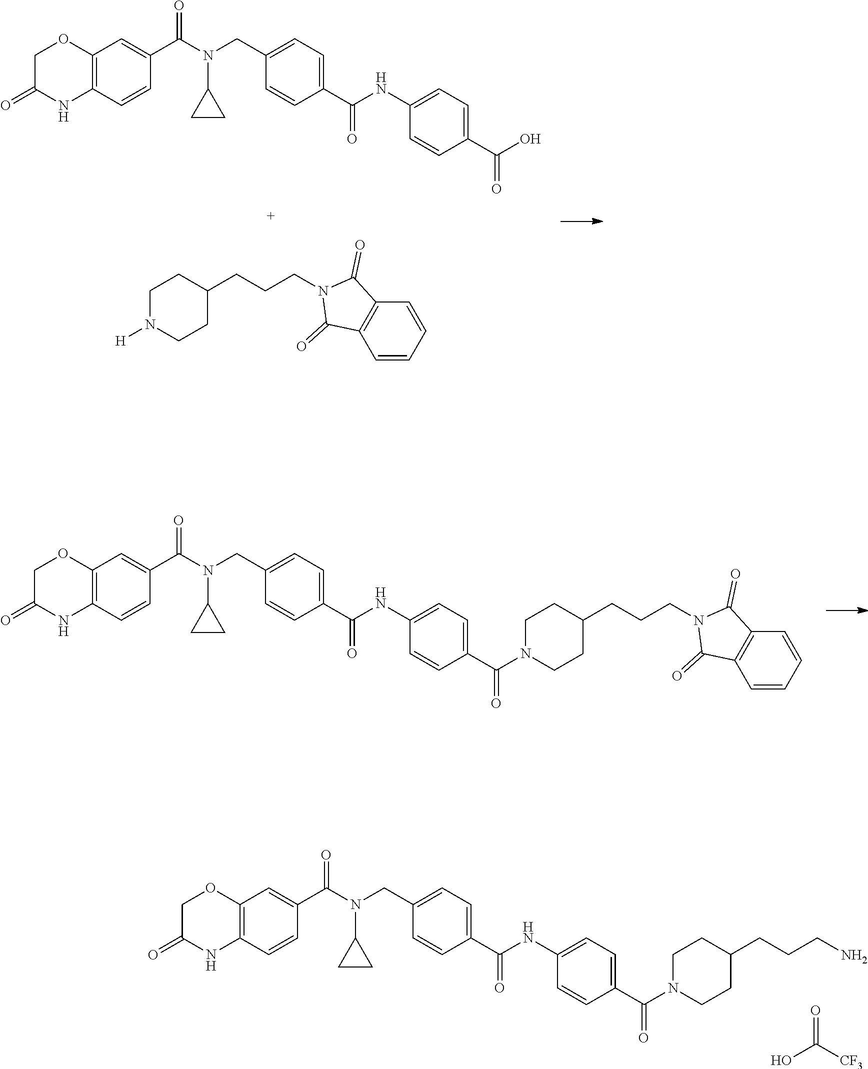 [SMILES: O=C1COc2cc(C(=O)N(Cc3ccc(C(=O)Nc4ccc(C(=O)O)cc4)cc3)C3CC3)ccc2N1 ; O=C1c2ccccc2C(=O)N1CCCC1CCNCC1 ; O=C1COc2cc(C(=O)N(Cc3ccc(C(=O)Nc4ccc(C(=O)N5CCC(CCCN6C(=O)c7ccccc7C6=O)CC5)cc4)cc3)C3CC3)ccc2N1 ; NCCCC1CCN(C(=O)c2ccc(NC(=O)c3ccc(CN(C(=O)c4ccc5c(c4)OCC(=O)N5)C4CC4)cc3)cc2)CC1 ; O=C(O)C(F)(F)F]

Amide coupling was performed according to General Procedure A/Workup A using 4-(4-((N-cyclopropyl-3-oxo-3,4-dihydro-2H-benzo[b][1,4]oxazine-7-carboxamido)methyl)benzamido) benzoic acid (50 mg, 1 Eq, 0.10 mmol), EDC (30 mg, 1.5 Eq, 0.15 mmol), HOAt (21 mg, 1.5 Eq, 0.15 mmol), DMF (0.5 mL) 2-(3-(piperidin-4-yl)propyl)isoindoline-1,3-dione (28 mg, 1 Eq, 0.10 mmol) and triethylamine (42 mg, 57 μL, 4 Eq, 0.41 mmol). To the phthalimide protected product was added 1 mL of aqueous methylamine and the mixture was left to stir overnight at room temperature. Volatiles were removed in vacuo and the crude material was purified by reverse phase chromatography (10-100% methanol in water+0.1% TFA) to give N-(4-((4-(4-(3-aminopropyl)piperidine-1-carbonyl)phenyl)carbamoyl)benzyl)-N-cyclopropyl-3-oxo-3,4-dihydro-2H-benzo[b][1,4]oxazine-7-carboxamide 2,2,2-trifluoroacetate (10.92 mg, 15.09 μmol, 15%) as a white solid. ¹H NMR (400 MHz, cd₃od) δ 7.76 (d, J=8.3 Hz, 2H), 7.63 (d, J=8.6 Hz, 2H), 7.30 (d, J=7.5 Hz, 2H), 7.23 (d, J=8.7 Hz, 2H), 7.06-6.86 (m, 2H), 6.76 (d, J=8.0 Hz, 1H), 4.63 (s, 3H), 4.42 (s, 3H), 3.63 (s, 1H), 2.93 (s, 1H), 2.73 (t, J=7.6 Hz, 2H), 2.70-2.55 (m, 2H), 1.65 (s, 1H), 1.60-1.36 (m, 4H), 1.32-1.12 (m, 2H), 1.12-0.83 (m, 2H), 0.61-0.41 (m, 2H), 0.41-0.10 (m, 2H). LCMS (ESI, +ve mode) expected m/z for [C₃₅H₄₀N₅O₅⁺] [M+H] 610.3, found 610.3.

### Example 83

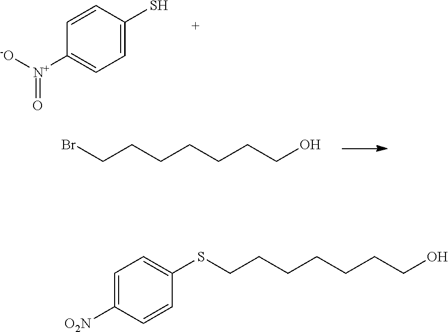

Prepared according to General Procedure E using 7-bromoheptan-1-ol (0.50 g, 1 Eq, 2.6 mmol), 4-nitrobenzenethiol (0.48 g, 1.2 Eq, 3.1 mmol), potassium carbonate (1.1 g, 3 Eq, 7.7 mmol), and DMF (3 mL). Normal phase chromatography over silica gel (0-25% EtOAc in hexanes) provided 7-((4-nitrophenyl)thio)heptan-1-ol (415.6 mg, 1.543 mmol, 60%) as a yellow solid. ¹H NMR (400 MHz, cdcl₃) δ 8.02 (d, J=9.0 Hz, 2H), 7.22 (d, J=9.0 Hz, 2H), 3.54 (t, J=6.6 Hz, 2H), 2.93 (t, J=7.4 Hz, 2H), 1.64 (p, J=7.4 Hz, 2H), 1.56-1.44 (m, 2H), 1.40 (p, J=6.8 Hz, 2H), 1.35-1.26 (m, 4H). LCMS (ESI, +ve mode) expected m/z for [C₁₃H₂₀NO₃S⁺] [M+H] 270.1, found 270.2.

### Example 84

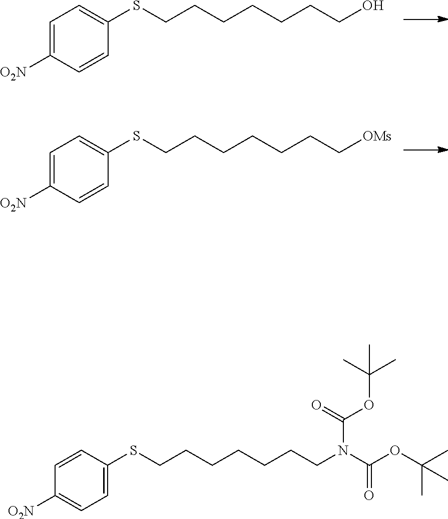 [SMILES: O=[N+]([O-])c1ccc(SCCCCCCCO)cc1 ; CS(=O)(=O)OCCCCCCCSc1ccc([N+](=O)[O-])cc1 ; CC(C)(C)OC(=O)N(CCCCCCCSc1ccc([N+](=O)[O-])cc1)C(=O)OC(C)(C)C]

Prepared according to General Procedure F using (step 1) 7-((4-nitrophenyl)thio)heptan-1-ol (415.6 mg, 1 Eq, 1.543 mmol), DCM (10 mL), triethylamine (281.0 mg, 387 μL, 1.8 Eq, 2.777 mmol), mesyl chloride (247.4 mg, 168.3 μL, 1.4 Eq, 2.160 mmol) and (step 2) di-tert-butyl iminodicarbonate (402.3 mg, 1.2 Eq, 1.851 mmol), Cs2CO3 (754.1 mg, 1.5 Eq, 2.314 mmol), and DMF (5 mL). Normal phase chromatography over silica gel (0-25% EtOAc in hexanes) provided tert-butyl (tert-butoxycarbonyl)(7-((4-nitrophenyl)thio)heptyl)carbamate (502.1 mg, 1.071 mmol, 69.44%) as a clear oil. ¹H NMR (400 MHz, cdcl₃) δ 8.02 (d, J=9.0 Hz, 2H), 7.23 (d, J=9.0 Hz, 2H), 3.55-3.39 (m, 2H), 2.93 (t, J=7.4 Hz, 2H), 1.64 (p, J=7.3 Hz, 2H), 1.53-1.47 (m, 2H), 1.42 (s, 18H), 1.38-1.10 (m, 6H). LCMS (ESI, +ve mode) expected m/z for [C₂₃H₃₇N₂O₆S⁺] [M+H] 469.2, found 469.3.

### Example 85

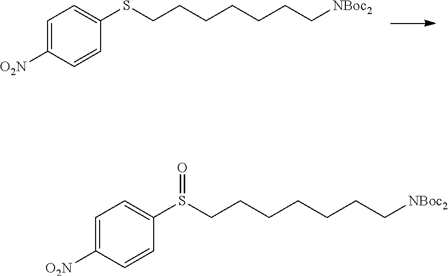

In a round-bottomed flask (50 mL) equipped with a magnetic stirrer, a solution of tert-butyl (tert-butoxycarbonyl)(7-((4-nitrophenyl)thio)heptyl)carbamate (221 mg, 1 Eq, 600 μmol) in EtOH (3 mL, 0.2) was prepared. Hydrogen peroxide (136 mg, 123 μL, 30% Wt, 2 Eq, 1.20 mmol) and trifluoromethanesulfonic anhydride (84.6 mg, 50.4 μL, 0.5 Eq, 300 μmol) were added and the mixture was stirred at room temperature overnight. The progress of the reaction was monitored by TLC. When the starting sulfane had completely disappeared, the mixture was quenched by adding H₂O (10 mL). The product was extracted with EtOAc (4×) and the combined extracts were dried (MgSO₄). Volatiles were removed in vacuo to give pure tert-butyl (tert-butoxycarbonyl)(7-((4-nitrophenyl)sulfinyl)heptyl)carbamate (101.7 mg, 209.9 umol, 35.0%). ¹H NMR (400 MHz, cdcl₃) δ 8.31-8.14 (m, 2H), 7.78-7.53 (m, 2H), 3.51-3.21 (m, 2H), 2.87-2.46 (m, 2H), 1.90-1.50 (m, 2H), 1.50-1.40 (m, 2H), 1.35 (s, 18H), 1.25-0.98 (m, 6H). LCMS (ESI, +ve mode) expected m/z for [C₂₃H₃₆N₂O₇SNa⁺] [M+Na] 507.2, found 507.1.

### Example 86

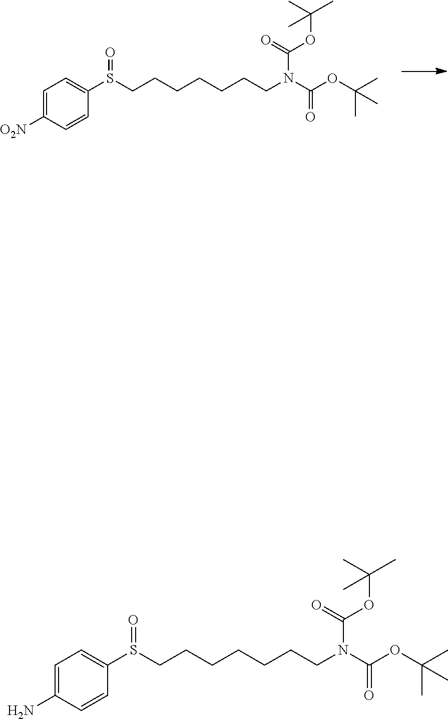 [SMILES: CC(C)(C)OC(=O)N(CCCCCCCS(=O)c1ccc([N+](=O)[O-])cc1)C(=O)OC(C)(C)C ; CC(C)(C)OC(=O)N(CCCCCCCS(=O)c1ccc(N)cc1)C(=O)OC(C)(C)C]

Prepared according to General Procedure C using tert-butyl (tert-butoxycarbonyl)(7-((4-nitrophenyl)sulfinyl)heptyl)carbamate (101.7 mg, 1 Eq, 209.9 μmol), ethyl acetate (10 mL) and Pd/C (11.17 mg, 10% Wt, 0.05 Eq, 10.49 μmol) to give a yellow oil. The crude material was purified by normal phase chromatography over silica gel (0-10% MeOH in DCM) to give tert-butyl (7-((4-aminophenyl)sulfinyl)heptyl)(tert-butoxycarbonyl)carbamate (57 mg, 0.13 mmol, 60%) as a yellow oil. ¹H NMR (400 MHz, cdcl₃) δ 7.30 (d, J=8.5 Hz, 2H), 6.65 (d, J=8.5 Hz, 2H), 3.46-3.41 (m, 2H), 2.76-2.56 (m, 2H), 1.60-1.50 (m, 2H), 1.50-1.44 (m, 2H), 1.40 (s, 18H), 1.36-1.20 (m, 6H). LCMS (ESI, +ve mode) expected m/z for [C₂₃H₃₈N₂O₅SNa⁺] [M+Na] 477.2, found 477.3.

### Example 87

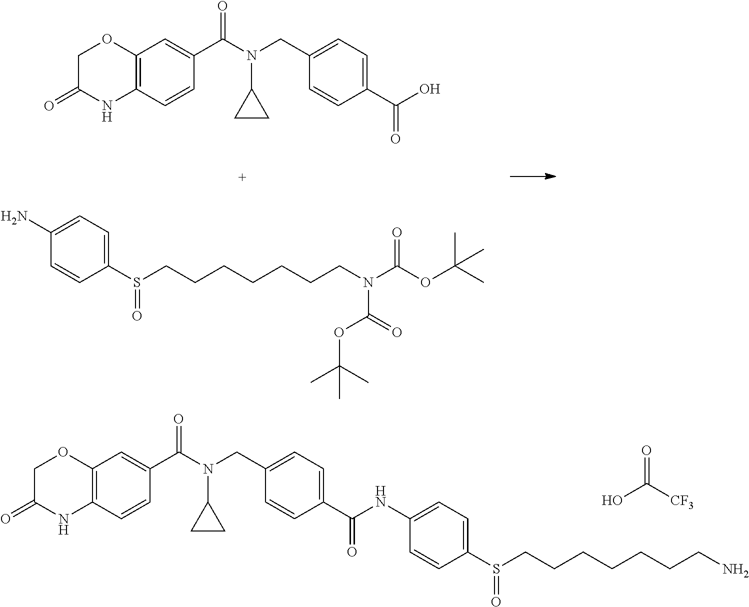 [SMILES: O=C1COc2cc(C(=O)N(Cc3ccc(C(=O)O)cc3)C3CC3)ccc2N1 ; CC(C)(C)OC(=O)N(CCCCCCCS(=O)c1ccc(N)cc1)C(=O)OC(C)(C)C ; NCCCCCCCS(=O)c1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (24 mg, 1 Eq, 66 μmol), EDC (19 mg, 1.5 Eq, 99 μmol), HOAt (13 mg, 1.5 Eq, 99 μmol), DMF (2 mL), tert-butyl (7-((4-aminophenyl)sulfinyl)heptyl)(tert-butoxycarbonyl)carbamate (57 mg, 1.9 Eq, 0.13 mmol) and triethylamine (27 mg, 37 μL, 4 Eq, 0.26 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA). The product was taken up in minimal methanol and triturated into ether to give N-(4-((4-((7-aminoheptyl)sulfinyl)phenyl)carbamoyl)benzyl)-N-cyclopropyl-3-oxo-3,4-dihydro-2H-benzo[b][1,4]oxazine-7-carboxamide 2,2,2-trifluoroacetate (5.83 mg, 8.13 μmol, 12%) as a white solid. ¹H NMR (400 MHz, cd₃od) δ 8.03-7.90 (m, 4H), 7.68 (d, J=8.8 Hz, 2H), 7.50 (d, J=7.6 Hz, 2H), 7.25-7.08 (m, 2H), 6.96 (d, J=8.0 Hz, 1H), 4.83 (s, 2H), 4.62 (s, 2H), 3.01-2.86 (m, 4H), 2.83 (s, 1H), 1.86-1.68 (m, 1H), 1.71-1.55 (m, 3H), 1.55-1.42 (m, 2H), 1.43-1.33 (m, 4H), 0.79-0.61 (m, 2H), 0.61-0.32 (m, 2H). LCMS (ESI, +ve mode) expected m/z for [C₃₃H₃₉N₄O₅S⁺] [M+H] 603.3, found 603.2.

### Example 88

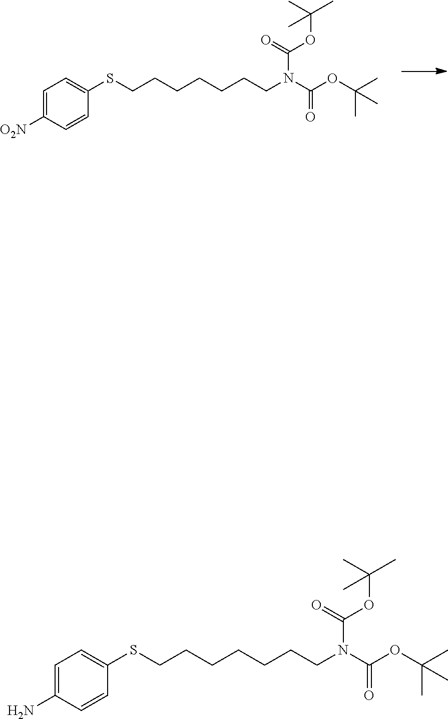 [SMILES: CC(C)(C)OC(=O)N(CCCCCCCSc1ccc([N+](=O)[O-])cc1)C(=O)OC(C)(C)C ; CC(C)(C)OC(=O)N(CCCCCCCSc1ccc(N)cc1)C(=O)OC(C)(C)C]

Prepared according to General Procedure C using tert-butyl (tert-butoxycarbonyl)(7-((4-nitrophenyl)thio)heptyl)carbamate (281.1 mg, 1 Eq, 599.9 μmol), ethyl acetate (10 mL) and Pd/C (31.92 mg, 10% Wt, 0.05 Eq, 29.99 μmol) to give a dark purple oil. The crude material was purified by normal phase chromatography over silica gel (0-10% MeOH in DCM) to give tert-butyl (7-((4-aminophenyl)thio)heptyl)(tert-butoxycarbonyl)carbamate (132.4 mg, 301.8 μmol, 50.32%) as a light purple oil. ¹H NMR (400 MHz, cd₃od) δ 7.15 (d, J=8.4 Hz, 2H), 6.64 (d, J=8.4 Hz, 2H), 3.56-3.51 (m, 2H), 2.71 (t, J=7.2 Hz, 2H), 1.59-1.52 (m, 4H), 1.49 (s, 18H), 1.44-1.29 (m, 6H). LCMS (ESI+ve mode) expected m/z for [C₂₃H₃₈N₂O₄SNa⁺] [M+Na] 461.2, found 461.2.

### Example 89

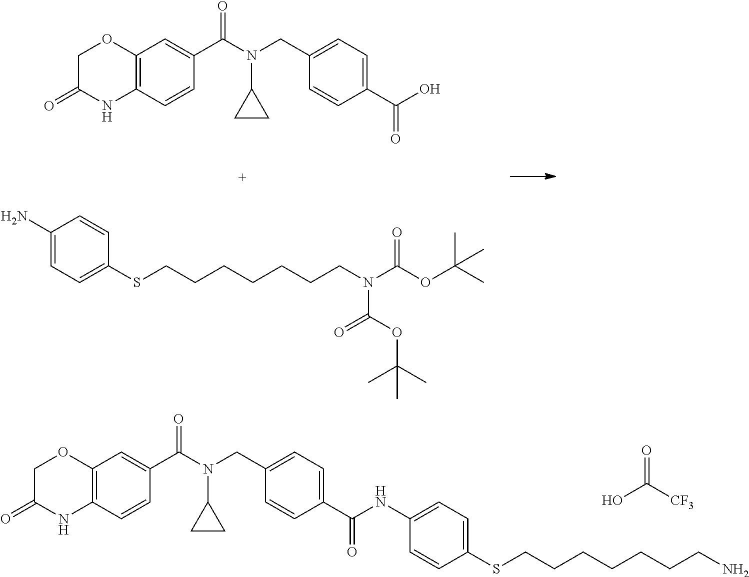 [SMILES: O=C1COc2cc(C(=O)N(Cc3ccc(C(=O)O)cc3)C3CC3)ccc2N1 ; CC(C)(C)OC(=O)N(CCCCCCCSc1ccc(N)cc1)C(=O)OC(C)(C)C ; NCCCCCCCSc1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (59.00 mg, 1 Eq, 161.0 μmol), EDC (46.30 mg, 1.5 Eq, 241.5 μmol), HOAt (32.88 mg, 1.5 Eq, 241.5 μmol), DMF (3 mL), tert-butyl (7-((4-aminophenyl)thio)heptyl)(tert-butoxycarbonyl)carbamate (134.2 mg, 1.9 Eq, 306.0 μmol) and triethylamine (65.18 mg, 89 μL, 4 Eq, 644.1 μmol). The N-Boc protected product was purified by normal phase chromatography over silica gel (0-10% MeOH in DCM). Following N-Boc deprotection, the product was purified by reverse phase chromatography (10-100% methanol in water+0.10% TFA). The product was taken up in minimal methanol and triturated into ether to give N-(4-((4-((7-aminoheptyl)thio)phenyl)carbamoyl)benzyl)-N-cyclopropyl-3-oxo-3,4-dihydro-2H-benzo[b][1,4]oxazine-7-carboxamide 2,2,2-trifluoroacetate (16.51 mg, 23.56 μmol, 14.63%) as a white solid. ¹H NMR (400 MHz, cd₃od) δ 7.93 (d, J=8.3 Hz, 2H), 7.64 (d, J=8.7 Hz, 2H), 7.48 (d, J=7.5 Hz, 2H), 7.39-7.26 (m, 2H), 7.26-7.07 (m, 2H), 6.95 (d, J=8.1 Hz, 1H), 4.82 (s, 2H), 4.62 (s, 2H), 2.91 (dt, J=12.0, 7.5 Hz, 4H), 2.82 (s, 1H), 1.64 (p, J=7.0 Hz, 4H), 1.56-1.42 (m, 2H), 1.42-1.24 (m, 4H), 0.79-0.60 (m, 2H), 0.60-0.35 (m, 2H). LCMS (ESI, +ve mode) expected m/z for [C₃₃H₃₉N₄O₄S⁺] [M+H] 587.3, found 587.2.

### Example 90

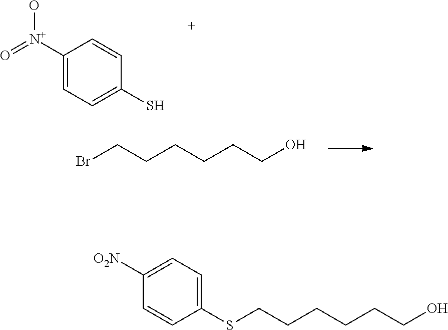

Prepared according to General Procedure E using 6-bromohexan-1-ol (0.50 g, 1 Eq, 2.8 mmol), 4-nitrobenzenethiol (0.51 g, 1.2 Eq, 3.3 mmol), potassium carbonate (1.1 g, 3 Eq, 8.3 mmol), and DMF (3 mL). Normal phase chromatography over silica gel (0-50% EtOAc in hexanes) provided 6-((4-nitrophenyl)thio)hexan-1-ol (391.4 mg, 1.533 mmol, 56%) as a yellow solid. ¹H NMR (400 MHz, cdcl₃) δ 8.09 (d, J=9.0 Hz, 2H), 7.29 (d, J=9.0 Hz, 2H), 3.63 (t, J=6.5 Hz, 2H), 3.00 (t, J=7.4 Hz, 2H), 1.72 (p, J=7.3 Hz, 2H), 1.61-1.53 (m, 2H), 1.52-1.45 (m, 2H), 1.45-1.30 (m, 2H). LCMS (ESI, +ve mode) expected m/z for [C₁₂H₁₈NO₃S⁺] [M+H] 256.1, found 256.1.

### Example 91

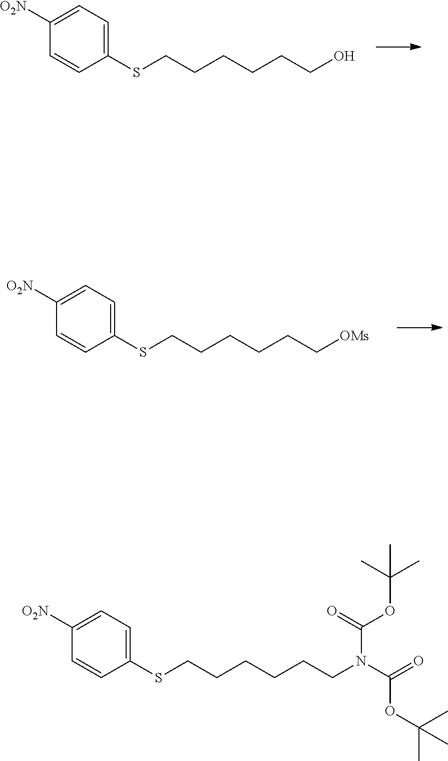 [SMILES: O=[N+]([O-])c1ccc(SCCCCCCO)cc1 ; CS(=O)(=O)OCCCCCCSc1ccc([N+](=O)[O-])cc1 ; CC(C)(C)OC(=O)N(CCCCCCSc1ccc([N+](=O)[O-])cc1)C(=O)OC(C)(C)C]

Prepared according to General Procedure F using (step 1) 6-((4-nitrophenyl)thio)hexan-1-ol (391.4 mg, 1 Eq, 1.533 mmol), DCM (10 mL), triethylamine (279.2 mg, 385 μL, 1.8 Eq, 2.759 mmol), mesyl chloride (245.8 mg, 167.2 μL, 1.4 Eq, 2.146 mmol) and (step 2) di-tert-butyl iminodicarbonate (399.7 mg, 1.2 Eq, 1.840 mmol), Cs2CO3 (749.2 mg, 1.5 Eq, 2.299 mmol), and DMF (5 mL). Normal phase chromatography over silica gel (0-25% EtOAc in hexanes) provided tert-butyl (tert-butoxy carbonyl)(6-((4-nitrophenyl)thio)hexyl)carbamate (382.2 mg, 840.8 μmol, 54.85%) as a clear oil. ¹H NMR (400 MHz, cdcl₃) δ 8.04 (d, J=9.0 Hz, 2H), 7.24 (d, J=9.0 Hz, 2H), 3.59-3.42 (m, 2H), 2.95 (t, J=7.4 Hz, 2H), 1.66 (p, J=7.4 Hz, 2H), 1.62-1.47 (m, 2H), 1.49-1.45 (m, 2H), 1.44 (s, 18H), 1.34-1.22 (m, 2H).

### Example 92

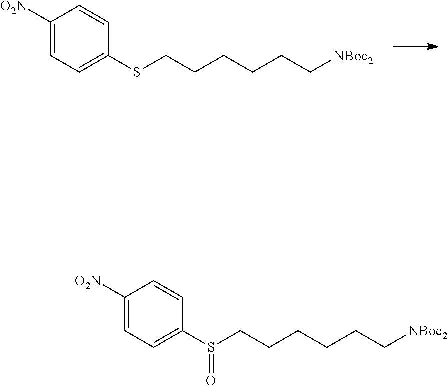

In a round-bottomed flask equipped with a magnetic stirrer, a solution of tert-butyl (tert-butoxycarbonyl)(6-((4-nitrophenyl)thio)hexyl)carbamate (188.8 mg, 1 Eq, 532.6 μmol) in EtOH (3 mL, 0.2) was prepared. Hydrogen peroxide (120.8 mg, 109 μL, 30% Wt, 2 Eq, 1.065 mmol) and trifluoromethanesulfonic anhydride (75.13 mg, 44.7 μL, 0.5 Eq, 266.3 μmol) were added and the mixture was stirred at room temperature for several hours. The progress of the reaction was monitored by TLC. When the starting sulfane had completely disappeared, the mixture was quenched by adding H₂O (10 mL). The product was extracted with EtOAc (4×) and the combined extracts were dried (MgSO4). Volatiles were removed in vacuo to give pure tert-butyl (tert-butoxycarbonyl)(6-((4-nitrophenyl)sulfinyl)hexyl)carbamate (134.3 mg, 285.4 μmol, 53.58%). ¹H NMR (400 MHz, cdcl₃) δ 8.36 (d, J=8.8 Hz, 2H), 7.79 (d, J=8.9 Hz, 2H), 3.61-3.38 (m, 2H), 2.91-2.68 (m, 2H), 1.90-1.75 (m, 1H), 1.66-1.57 (m, 1H), 1.57-1.48 (m, 4H), 1.47 (s, 18H), 1.32-1.25 (m, 2H).

### Example 93

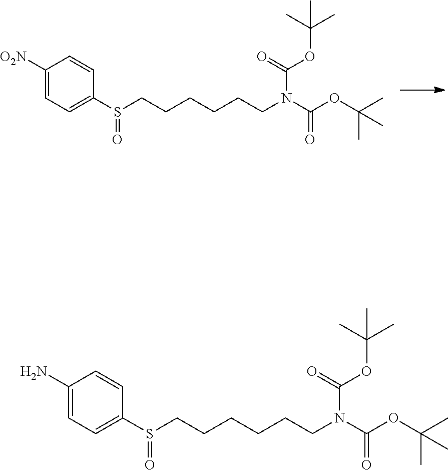 [SMILES: CC(C)(C)OC(=O)N(CCCCCCS(=O)c1ccc([N+](=O)[O-])cc1)C(=O)OC(C)(C)C ; CC(C)(C)OC(=O)N(CCCCCCS(=O)c1ccc(N)cc1)C(=O)OC(C)(C)C]

Prepared according to General Procedure C using tert-butyl (tert-butoxycarbonyl)(6-((4-nitrophenyl)sulfinyl)hexyl)carbamate (134.3 mg, 1 Eq, 285.4 μmol), ethyl acetate (10 mL) and Pd/C (15.19 mg, 10% Wt, 0.05 Eq, 14.27 μmol) to give a yellow oil. The crude material was purified by normal phase chromatography over silica gel (0-10% MeOH in DCM) to give tert-butyl (6-((4-aminophenyl)sulfinyl)hexyl)(tert-butoxycarbonyl)carbamate (61.1 mg, 139 μmol, 48.6%) as a yellow oil. ¹H NMR (400 MHz, cdcl₃) δ 7.34 (d, J=8.4 Hz, 2H), 6.69 (d, J=8.4 Hz, 2H), 3.47 (t, J=7.4 Hz, 2H), 2.88-2.72 (m, 1H), 2.72-2.47 (m, 1H), 1.69-1.56 (m, 2H), 1.57-1.50 (m, 2H), 1.44 (s, 18H), 1.40-1.30 (m, 4H).

### Example 94

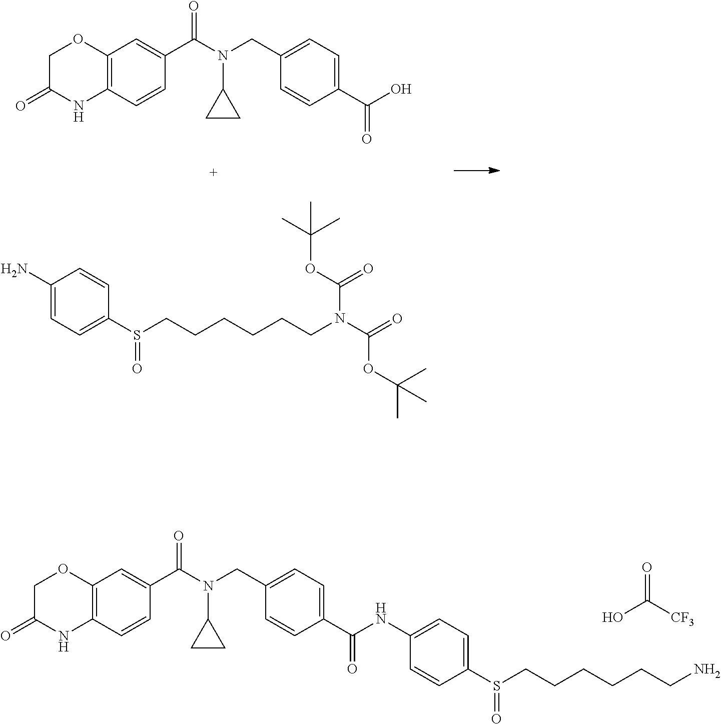 [SMILES: O=C1COc2cc(C(=O)N(Cc3ccc(C(=O)O)cc3)C3CC3)ccc2N1 ; CC(C)(C)OC(=O)N(CCCCCCS(=O)c1ccc(N)cc1)C(=O)OC(C)(C)C ; NCCCCCCS(=O)c1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (26.7 mg, 1 Eq, 73.0 μmol), EDC (21.0 mg, 1.5 Eq, 109 μmol), HOAt (14.9 mg, 1.5 Eq, 109 μmol), DMF (2 mL) tert-butyl (6-((4-aminophenyl)sulfinyl)hexyl)(tert-butoxycarbonyl)carbamate (61.1 mg, 1.9 Eq, 139 μmol) and triethylamine (29.5 mg, 40 μL, 4 Eq, 292 μmol). The N-Boc protected product was purified by normal phase chromatography over silica gel (0-10% MeOH in DCM). Following N-Boc deprotection, the product was purified by reverse phase chromatography (10-100% methanol in water+0.1% TFA). The product was taken up in minimal methanol and triturated into ether to give N-(4-((4-((6-aminohexyl)sulfinyl)phenyl)carbamoyl)benzyl)-N-cyclopropyl-3-oxo-3,4-dihydro-2H-benzo[b][1,4]oxazine-7-carboxamide 2,2,2-trifluoroacetate (11.17 mg, 15.89 μmol, 21.8%) as a white solid. ¹H NMR (400 MHz, cd₃od) δ 8.09-7.82 (m, 4H), 7.68 (d, J=8.8 Hz, 2H), 7.50 (d, J=7.5 Hz, 2H), 7.28-7.09 (m, 2H), 6.96 (d, J=8.1 Hz, 1H), 4.83 (s, 2H), 4.62 (s, 2H), 3.05-2.86 (m, 4H), 2.83 (s, 1H), 1.88-1.69 (m, 1H), 1.71-1.58 (m, 3H), 1.58-1.45 (m, 2H), 1.47-1.36 (m, 2H), 0.87-0.62 (m, 2H), 0.61-0.29 (m, 2H). LCMS (ESI, +ve mode) expected m/z for [C₃₂H₃₇N₄O₅S⁺] [M+H] 589.2, found 589.2.

### Example 95

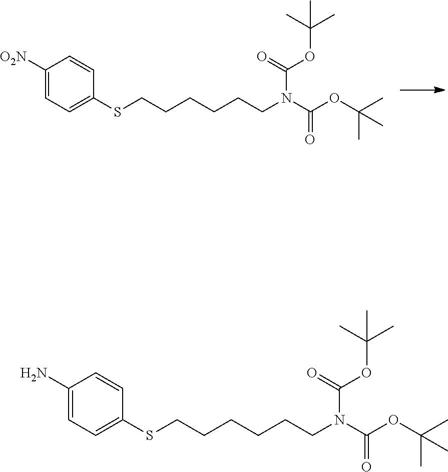 [SMILES: CC(C)(C)OC(=O)N(CCCCCCSc1ccc([N+](=O)[O-])cc1)C(=O)OC(C)(C)C ; CC(C)(C)OC(=O)N(CCCCCCSc1ccc(N)cc1)C(=O)OC(C)(C)C]

Prepared according to General Procedure C using tert-butyl (tert-butoxycarbonyl)(6-((4-nitrophenyl)thio)hexyl)carbamate (193.4 mg, 1 Eq, 425.4 μmol), ethyl acetate (10 mL) and Pd/C (22.64 mg, 10% Wt, 0.05 Eq, 21.27 μmol) to give a bright yellow solid. The crude material was purified by normal phase chromatography over silica gel (0-10% MeOH in DCM) to give tert-butyl (6-((4-aminophenyl)thio)hexyl)(tert-butoxycarbonyl)carbamate (156.2 mg, 367.9 μmol, 86.47%) as a yellow oil. ¹H NMR (400 MHz, cdcl₃) δ 7.21 (d, J=8.6 Hz, 2H), 6.60 (d, J=8.6 Hz, 2H), 3.56-3.48 (m, 2H), 2.78-2.62 (m, 2H), 1.61-1.51 (m, 4H), 1.49 (s, 18H), 1.45-1.31 (m, 2H), 1.31-1.04 (m, 2H).

### Example 96

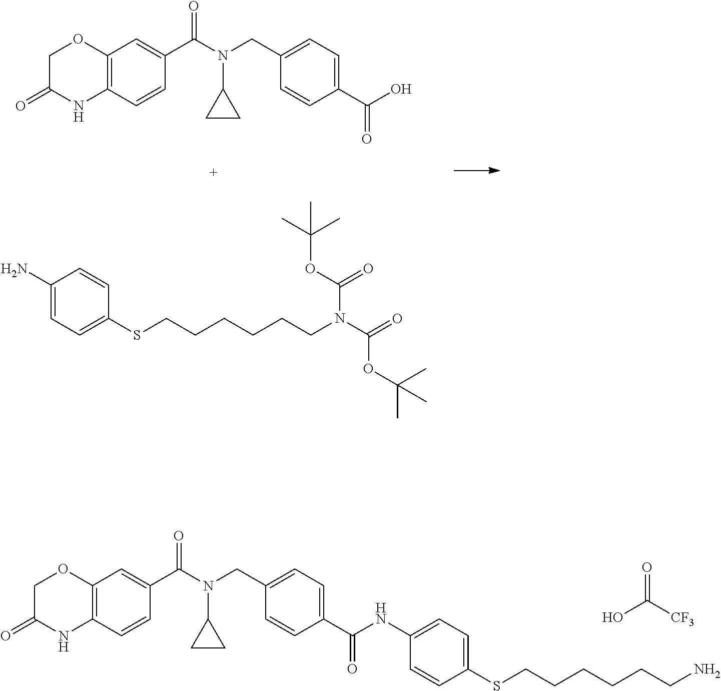 [SMILES: O=C1COc2cc(C(=O)N(Cc3ccc(C(=O)O)cc3)C3CC3)ccc2N1 ; CC(C)(C)OC(=O)N(CCCCCCSc1ccc(N)cc1)C(=O)OC(C)(C)C ; NCCCCCCSc1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (70.94 mg, 1 Eq, 193.6 μmol), EDC (55.68 mg, 1.5 Eq, 290.4 μmol), HOAt (39.53 mg, 1.5 Eq, 290.4 μmol), DMF (3 mL), tert-butyl (6-((4-aminophenyl)thio)hexyl)(tert-butoxycarbonyl)carbamate (156.2 mg, 1.9 Eq, 367.9 μmol) and triethylamine (78.37 mg, 0.11 mL, 4 Eq, 774.5 μmol). The N-Boc protected product was purified by normal phase chromatography over silica gel (0-10% MeOH in DCM). Following N-Boc deprotection, the product was purified by reverse phase chromatography (10-100% methanol in water+0.1% TFA). The product was taken up in minimal methanol and triturated into ether to give N-(4-((4-((6-aminohexyl)thio)phenyl)carbamoyl)benzyl)-N-cyclopropyl-3-oxo-3,4-dihydro-2H-benzo[b][1,4]oxazine-7-carboxamide 2,2,2-trifluoroacetate (14.33 mg, 20.87 μmol, 10.78%) as a white solid. ¹H NMR (400 MHz, cd₃od) δ 7.93 (d, J=8.2 Hz, 2H), 7.64 (d, J=8.7 Hz, 2H), 7.48 (d, J=7.4 Hz, 2H), 7.35 (d, J=8.7 Hz, 2H), 7.23-7.09 (m, 2H), 6.95 (d, J=8.1 Hz, 1H), 4.82 (s, 2H), 4.61 (s, 2H), 2.91 (dt, J=16.3, 7.4 Hz, 4H), 2.82 (s, 1H), 1.64 (h, J=7.8 Hz, 4H), 1.50 (dt, J=14.0, 6.8 Hz, 2H), 1.45-1.28 (m, 2H), 0.85-0.60 (m, 2H), 0.60-0.18 (m, 2H). LCMS (ESI, +ve mode) expected m/z for [C₃₂H₃₇N₄O₄S⁺] [M+H] 573.3, found 573.2.

### Example 97

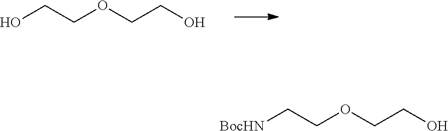 [SMILES: OCCOCCO ; CC(C)(C)OC(=O)NCCOCCO]

To a 100 mL RBF was added 2-(2-aminoethoxy)ethan-1-ol (1.5 g, 1.4 mL, 1 Eq, 14 mmol), sodium bicarbonate (3.6 g, 3 Eq, 43 mmol), and water (20 mL). The mixture was cooled to 0° Celcius. A solution of boc anhydride (3.7 g, 4 mL, 1.2 Eq, 17 mmol) in THF (20 mL) was added, and the reaction stirred overnight. The reaction was extracted three times with ethyl acetate. The organics were combined and washed with brine, dried over magnesium sulfate, filtered and concentrated in vacuo to a clear oil. The oil was purified by silica gel chromatography (0-100% EtOAc in Hexanes) to give tert-butyl (2-(2-hydroxyethoxy)ethyl)carbamate (2.4617 g, 11.994 mmol, 84%). ¹H NMR (400 MHz, cdcl₃) δ 4.91 (br s, 1H, OH), 3.80-3.66 (m, 2H), 3.56 (dt, J=10.1, 5.0 Hz, 4H), 3.33 (q, J=5.1 Hz, 2H), 2.13 (br s, 1H, NH), 1.44 (s, 9H). LCMS (ESI, +ve mode) expected m/z for [C₉H₁₉NO₄Na⁺][M+Na] 228.1, found 228.1.

### Example 98

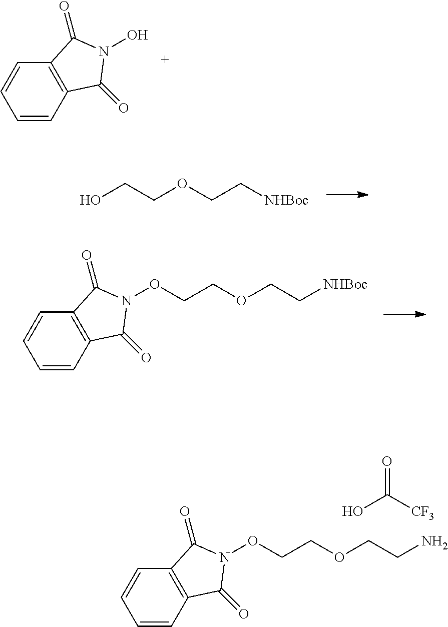

A 100 mL flask was evacuated and backfilled with nitrogen three times. Then 2-hydroxyisoindoline-1,3-dione (2.9348 g, 1.5 Eq, 17.990 mmol), triphenylphosphine (4.7187 g, 1.5 Eq, 17.990 mmol), and tert-butyl (2-(2-hydroxyethoxy)ethyl)carbamate (2.4617 g, 1 Eq, 11.994 mmol) were added. The solids were dissolved with stirring in anhydrous toluene (50 mL) and the flask immersed in an ice/water bath for 10 minutes. Next, di-tert-butyl (E)-diazene-1,2-dicarboxylate (4.1425 g, 3.98 mL, 1.5 Eq, 17.990 mmol) was added dropwise. The bright orange reagent disappeared and the reaction briefly took on a pale yellow hue. The reaction was left to stir overnight. Toluene was removed in vacuo, and the residue was diluted with ethyl acetate and washed twice with saturated sodium bicarbonate, once with brine, then dried over sodium sulfate and concentrated to a pale yellow residue. Normal phase chromatography on silica gel was performed three times (0-100% EtOAc in hexanes, 0-30% EtOAc in hexanes, 100% DCM) to give semi-purified di-protected product. Following N-Boc deprotection the product was purified twice using normal phase chromatography on silica gel (0-15% MeOH in DCM) to give 2-(2-(2-aminoethoxy)ethoxy)isoindoline-1,3-dione 2,2,2-trifluoroacetate (997.3 mg, 2.738 mmol, 46%) as a viscous yellow oil. ¹H NMR (400 MHz, cd₃od) δ7.89-7.56 (m, 4H), 4.87 (br s, 2H, NH₂), 4.47-4.25 (m, 2H), 3.94-3.65 (m, 4H), 3.30-3.05 (m, 2H). LCMS (ESI, +ve mode) expected m/z for [C₁₂H₁₅N₂O₄⁺] [M+H] 251.1 found 251.1.

### Example 99

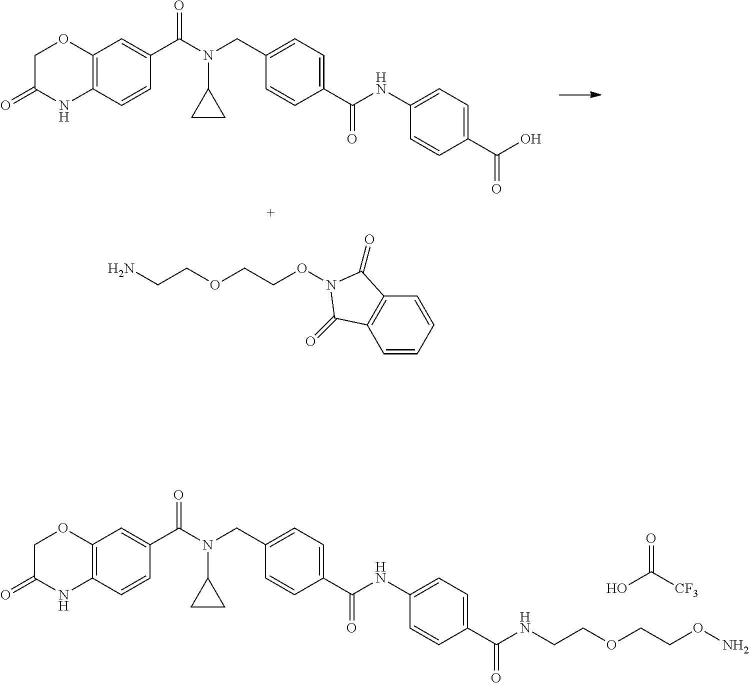

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), 2-(2-(2-aminoethoxy)ethoxy)isoindoline-1,3-dione 2,2,2-trifluoroacetate (56 mg, 1.5 Eq, 0.15 mmol) and triethylamine (42 mg, 57 μL, 4 Eq, 0.41 mmol). The phthalimide was removed by stirring in 1 mL of aqueous methylamine overnight. Volatiles were removed in vacuo and product was purified by reverse phase chromatography (10-100% methanol in water+0.1% TFA) to give N-(4-((4-((2-(2-(aminooxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (0.99 mg, 1.4 μmol, 3.3%) as a white solid. ¹H NMR (400 MHz, cd₃od) δ 7.95 (d, J=8.3 Hz, 2H), 7.90-7.77 (m, 4H), 7.50 (d, J=7.6 Hz, 2H), 7.27-7.08 (m, 2H), 6.96 (d, J=8.0 Hz, 1H), 4.83 (s, 2H), 4.62 (s, 2H), 4.24-4.15 (m, 2H), 3.88-3.76 (m, 2H), 3.69 (t, J=5.3 Hz, 2H), 3.62 (t, J=5.2 Hz, 2H), 2.83 (s, 1H), 0.81-0.60 (m, 2H), 0.61-0.34 (m, 2H). LCMS (ESI, +ve mode) expected m/z for [C₃₁H₃₄N₅O₇⁺] [M+H] 588.2, found 588.2.

### Example 100

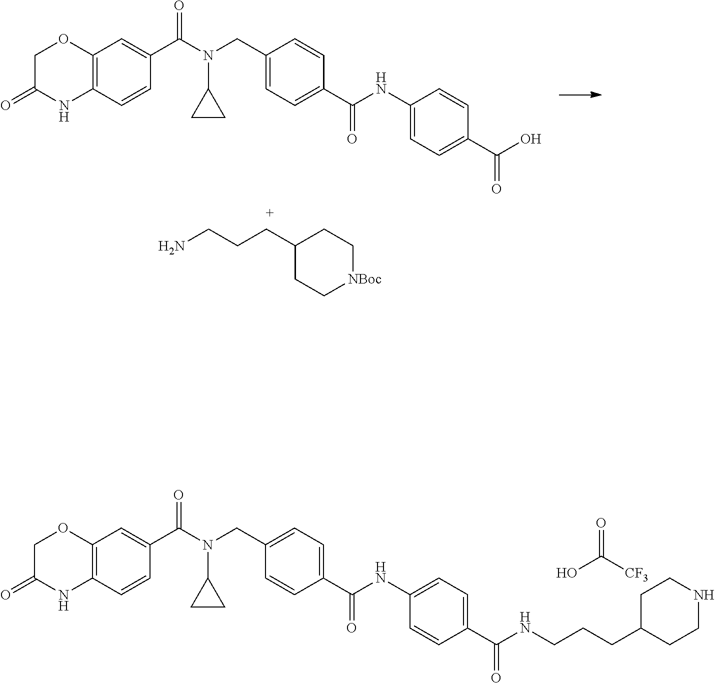

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), tert-butyl 4-(3-aminopropyl)piperidine-1-carboxylate (25 mg, 1 Eq, 0.10 mmol) and triethylamine (42 mg, 57 μL, 4 Eq, 0.41 mmol). Following N-Boc deprotection, the product was purified by reverse phase chromatography (10-100% methanol in water+0.1% TFA) to give N-cyclopropyl-3-oxo-N-(4-((4-((3-(piperidin-4-yl)propyl)carbamoyl)phenyl)carbamoyl)benzyl)-3,4-dihydro-2H-benzo[b][1,4]oxazine-7-carboxamide 2,2,2-trifluoroacetate (17.32 mg, 23.93 μmol, 23%) as a white solid. ¹H NMR (400 MHz, cd₃od) δ 7.95 (d, J=8.3 Hz, 2H), 7.84 (s, 4H), 7.49 (d, J=7.3 Hz, 2H), 7.28-7.09 (m, 2H), 6.96 (d, J=8.0 Hz, 1H), 4.82 (s, 2H), 4.62 (s, 2H), 3.45-3.33 (m, 4H), 3.04-2.89 (m, 2H), 2.82 (s, 1H), 1.97 (d, J=14.5 Hz, 2H), 1.78-1.54 (m, 3H), 1.48-1.15 (m, 4H), 0.84-0.60 (m, 2H), 0.60-0.32 (m, 2H). LCMS (ESI, +ve mode) expected m/z for [C₃₅H₄₀N₅O₅⁺] [M+H] 610.3, found 610.2.

### Example 101

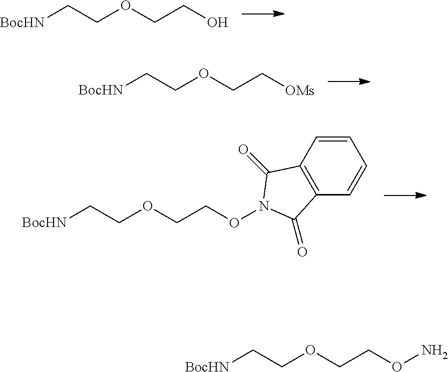 [SMILES: CC(C)(C)OC(=O)NCCOCCO ; CC(C)(C)OC(=O)NCCOCCOS(C)(=O)=O ; CC(C)(C)OC(=O)NCCOCCON1C(=O)c2ccccc2C1=O ; CC(C)(C)OC(=O)NCCOCCON]

To a flask containing tert-butyl (2-(2-hydroxyethoxy)ethyl)carbamate (2.5224 g, 1 Eq, 12.289 mmol) was added DCM (60 mL) and triethylamine (2.2384 g, 3.08 mL, 1.8 Eq, 22.121 mmol). The flask was cooled in an ice bath, and mesyl chloride (1.9707 g, 1.341 mL, 1.4 Eq, 17.205 mmol) was added dropwise. The reaction was allowed to come to room temperature with stirring overnight. The reaction mixture was partitioned between 1M HCl and DCM, and the aqueous layer extracted twice more with DCM. The combined organic layers were washed once with water, once with saturated sodium bicarbonate, and once with brine, then dried over sodium sulfate and concentrated to a yellow oil. NMR confirmed conversion to the mesylate.

To the flask containing the mesylate was added potassium carbonate (5.0952 g, 3 Eq, 36.868 mmol) and 2-hydroxyisoindoline-1,3-dione (2.0048 g, 1 Eq, 12.289 mmol), followed by DMF (30 mL). The reaction was heated to 70° C. overnight. The reaction was cooled and diluted with water. The aqueous solution was extracted three times with ethyl acetate, and the combined organic layers washed three times with water and once with brine, then dried over sodium sulfate and concentrated to a yellow oil. Normal phase chromatography over silica gel (0-25% ethyl acetate in hexanes) provided the di-protected product as a clear oil. The oil was stirred with 5 mL of aqueous methylamine overnight. Solvents were removed in vacuo and the resulting product was purified by normal phase chromatography over silica gel (0-10% MeOH in DCM) to give tert-butyl (2-(2-(aminooxy)ethoxy)ethyl)carbamate (205 mg, 931 μmol, 7.57%). ¹H NMR (400 MHz, cdcl₃) δ 5.16 (br s, 3H, NH, NH₂), 3.79-3.71 (m, 2H), 3.61-3.53 (m, 2H), 3.46 (t, J=5.2 Hz, 2H), 3.32-3.09 (m, 2H), 1.37 (s, 9H). LCMS (ESI, +ve mode) expected m/z for [C₉H₂₀N₂O₄Na⁺] [M+Na] 243.1, found 243.1.

### Example 102

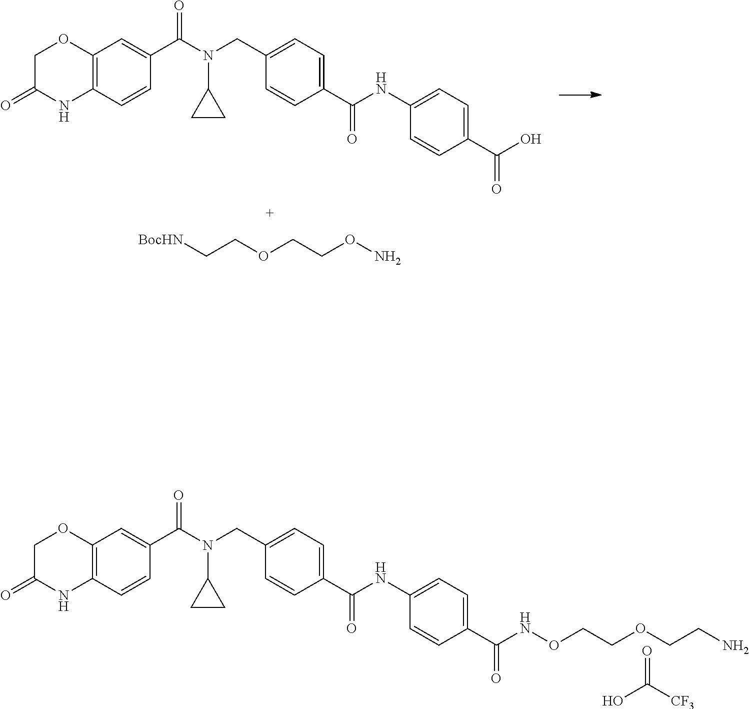

Prepared according to General Procedure A/Workup A using 4-(4-((N-cyclopropyl-3-oxo-3,4-dihydro-2H-benzo[b][1,4]oxazine-7-carboxamido)methyl)benzamido)benzoic acid (150 mg, 1 Eq, 309 μmol), EDC (88.8 mg, 1.5 Eq, 463 μmol), HOAt (63.1 mg, 1.5 Eq, 463 μmol), DMF (3 mL) tert-butyl (2-(2-(aminooxy)ethoxy)ethyl)carbamate (102 mg, 1.5 Eq, 463 μmol) and triethylamine (125 mg, 0.17 mL, 4 Eq, 1.24 mmol). Following N-Boc deprotection, the product was purified by reverse phase chromatography (10-100% methanol in water+0.1% TFA) to give N-(4-((4-((2-(2-aminoethoxy)ethoxy)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (50.64 mg, 72.17 μmol, 23.4%) as a white solid. ¹H NMR (400 MHz, cd₃od) δ 7.94 (d, J=8.3 Hz, 2H), 7.89-7.82 (m, 2H), 7.82-7.69 (m, 2H), 7.48 (d, J=7.7 Hz, 2H), 7.25-7.07 (m, 2H), 6.95 (d, J=8.0 Hz, 1H), 4.87 (s, 2H), 4.81 (s, 2H), 4.20-4.05 (m, 2H), 3.85-3.67 (m, 4H), 3.21-3.08 (m, 2H), 2.81 (s, 1H), 0.86-0.58 (m, 2H), 0.59-0.28 (m, 2H). LCMS (ESI, +ve mode) expected m/z for [C₃₁H₃₄N₅O₇⁺] [M+H] 588.2, found 588.2.

### Example 103

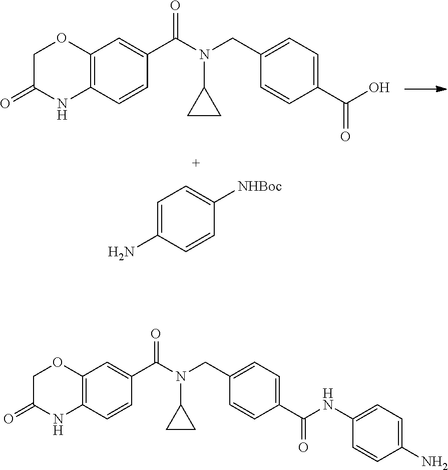 [SMILES: O=C1COc2cc(C(=O)N(Cc3ccc(C(=O)O)cc3)C3CC3)ccc2N1 ; CC(C)(C)OC(=O)Nc1ccc(N)cc1 ; Nc1ccc(NC(=O)c2ccc(CN(C(=O)c3ccc4c(c3)OCC(=O)N4)C3CC3)cc2)cc1]

Prepared according to General Procedure A/Workup A using 4-((N-cyclopropyl-3-oxo-3,4-dihydro-2H-benzo[b][1,4]oxazine-7-carboxamido)methyl)benzoic acid (123.0 mg, 1 Eq, 335.6 μmol), EDC (96.51 mg, 1.5 Eq, 503.4 μmol), HOAt (68.52 mg, 1.5 Eq, 503.4 μmol), DMF (4 mL), tert-butyl (4-aminophenyl)carbamate (132.8 mg, 1.9 Eq, 637.7 μmol) and triethylamine (135.8 mg, 0.19 mL, 4 Eq, 1.342 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-aminophenyl)carbamoyl)benzyl)-N-cyclopropyl-3-oxo-3,4-dihydro-2H-benzo[b][1,4]oxazine-7-carboxamide (143 mg, 313 μmol, 93.3%) as a beige colored solid. ¹H NMR (400 MHz, cd₃od) δ 7.94 (d, J=8.3 Hz, 2H), 7.88 (d, J=8.9 Hz, 2H), 7.47 (d, J=7.4 Hz, 2H), 7.43-7.25 (m, 2H), 7.25-7.06 (m, 2H), 6.94 (d, J=8.1 Hz, 1H), 4.80 (s, 2H), 4.59 (s, 2H), 2.81 (s, 1H), 0.80-0.59 (m, 2H), 0.58-0.25 (m, 2H). LCMS (ESI, +ve mode) expected m/z for [C₂₆H₂₅N₄O₄⁺] [M+H] 457.2, found 457.2.

### Example 104

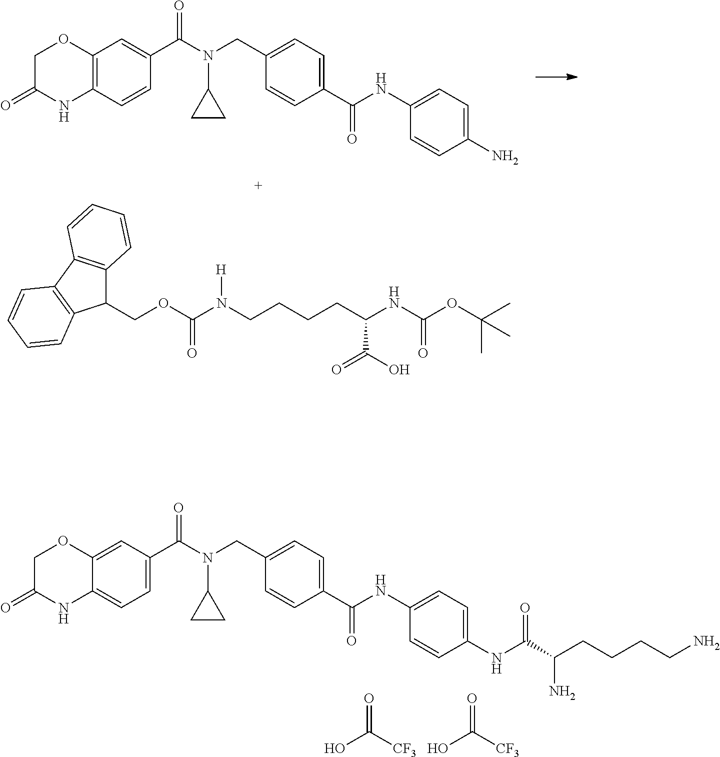

Prepared according to General Procedure B using N6-(((9H-fluoren-9-yl)methoxy)carbonyl)-N2-(tert-butoxycarbonyl)-L-lysine (150 mg, 1 Eq, 321 μmol), N-(4-((4-aminophenyl)carbamoyl)benzyl)-N-cyclopropyl-3-oxo-3,4-dihydro-2H-benzo[b][1,4]oxazine-7-carboxamide 2,2,2-trifluoroacetate (183 mg, 1 Eq, 321 μmol), 2-(1H-benzo[d][1,2,3]triazol-1-yl)-1,1,3,3-tetramethylisouronium tetrafluoroborate (134 mg, 1.3 Eq, 417 μmol), N-ethyl-N-isopropylpropan-2-amine (137 mg, 184 μL, 3.3 Eq, 1.06 mmol) and DMF. The FMOC group was removed by stirring with 1 mL of 20% piperidine in DMF overnight. Solvent was blown off under a flow of air to give an orange solid. Following N-Boc deprotection, volatiles were removed in vacuo and the crude mixture was purified by reverse phase chromatography to give (S)—N-cyclopropyl-N-(4-((4-(2,6-diaminohexanamido)phenyl)carbamoyl)benzyl)-3-oxo-3,4-dihydro-2H-benzo[b][1,4]oxazine-7-carboxamide bis(2,2,2-trifluoroacetate) (31.36 mg, 38.59 μmol, 12.0%) as a white solid. ¹H NMR (400 MHz, cd₃od) δ 7.98-7.92 (m, 2H), 7.69 (d, J=9.0 Hz, 2H), 7.62 (d, J=9.0 Hz, 2H), 7.48 (d, J=7.3 Hz, 2H), 7.26-7.09 (m, 2H), 6.95 (d, J=8.1 Hz, 1H), 4.82 (s, 2H), 4.61 (s, 2H), 4.03 (t, J=6.5 Hz, 1H), 2.98-2.89 (m, 2H), 2.82 (s, 1H), 2.13-1.88 (m, 2H), 1.73 (p, J=7.6 Hz, 2H), 1.63-1.39 (m, 2H), 0.79-0.60 (m, 2H), 0.60-0.28 (m, 2H). LCMS (ESI, +ve mode) expected m/z for [C₃₂H₃₈N₆O₅⁺] [M+2]586.3, found 586.3.

### Example 105

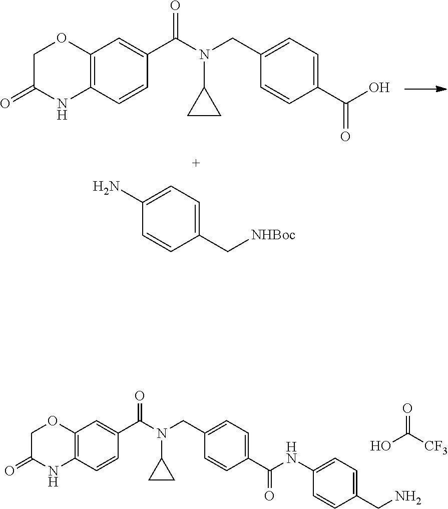 [SMILES: O=C1COc2cc(C(=O)N(Cc3ccc(C(=O)O)cc3)C3CC3)ccc2N1 ; CC(C)(C)OC(=O)NCc1ccc(N)cc1 ; NCc1ccc(NC(=O)c2ccc(CN(C(=O)c3ccc4c(c3)OCC(=O)N4)C3CC3)cc2)cc1 ; O=C(O)C(F)(F)F]

Prepared according to General Procedure A/Workup A using 4-((N-cyclopropyl-3-oxo-3,4-dihydro-2H-benzo[b][1,4]oxazine-7-carboxamido)methyl)benzoic acid (115.2 mg, 1 Eq, 314.4 μmol), EDC (90.41 mg, 1.5 Eq, 471.6 μmol), HOAt (64.20 mg, 1.5 Eq, 471.6 μmol), DMF (4 mL), tert-butyl (4-aminobenzyl)carbamate (132.8 mg, 1.9 Eq, 597.4 μmol) and triethylamine (127.3 mg, 0.17 mL, 4 Eq, 1.258 mmol). The N-Boc protected product was purified by normal phase chromatography over silica gel (0-10% MeOH in DCM). Following N-Boc deprotection, the product was purified by reverse phase chromatography (10-100% methanol in water+0.1% TFA) to give N-(4-((4-(aminomethyl)phenyl)carbamoyl)benzyl)-N-cyclopropyl-3-oxo-3,4-dihydro-2H-benzo[b][1,4]oxazine-7-carboxamide 2,2,2-trifluoroacetate (79 mg, 0.14 mmol, 43%) as a beige colored solid. ¹H NMR (400 MHz, cd₃od) δ 7.94 (d, J=8.3 Hz, 2H), 7.81 (d, J=8.5 Hz, 2H), 7.47 (dd, J=16.6, 7.7 Hz, 4H), 7.32-7.10 (m, 2H), 6.96 (d, J=8.1 Hz, 1H), 4.83 (s, 5H), 4.62 (s, 2H), 4.10 (s, 2H), 2.83 (s, 1H), 0.83-0.61 (m, 2H), 0.61-0.33 (m, 2H). LCMS (ESI, +ve mode) expected m/z for [C₂₇H₂₇N₄O₄⁺] [M+H] 471.2, found 471.2.

### Example 106

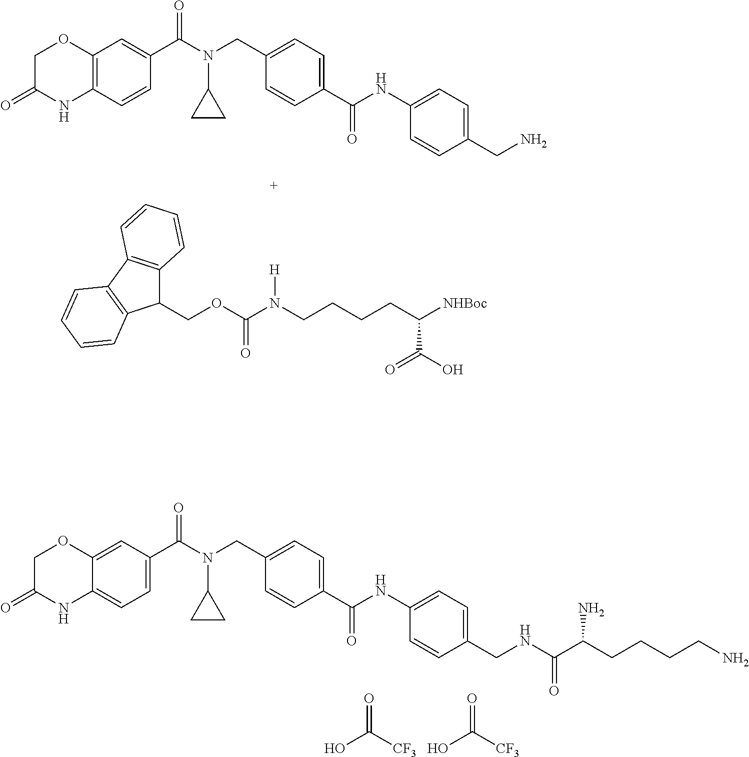

Prepared according to General Procedure B/Workup A using N6-(((9H-fluoren-9-yl)methoxy)carbonyl)-N2-(tert-butoxycarbonyl)-L-lysine (70 mg, 1.1 Eq, 0.15 mmol), TBTU (56 mg 1.3 Eq, 0.18 mmol), DMF (2 mL), N-(4-((4-(aminomethyl)phenyl)carbamoyl)benzyl)-N-cyclopropyl-3-oxo-3,4-dihydro-2H-benzo[b][1,4]oxazine-7-carboxamide 2,2,2-trifluoroacetate (79 mg, 1 Eq, 0.14 mmol) and DIPEA (58 mg, 78 μL, 3.3 Eq, 0.45 mmol). The FMOC group was removed by stirring with 1 mL of 20% piperidine in DMF overnight. Solvent was blown off under a flow of air to give a ruby colored residue. Following N-Boc deprotection the product was purified by recrystallization from minimal amount of boiling methanol to give (R)—N-cyclopropyl-N-(4-((4-((2,6-diaminohexanamido)methyl)phenyl)carbamoyl)benzyl)-3-oxo-3,4-dihydro-2H-benzo[b][1,4]oxazine-7-carboxamide bis(2,2,2-trifluoroacetate) (27.27 mg, 32.98 μmol, 24%) as a white solid. ¹H NMR (400 MHz, cd₃od) δ 7.94 (d, J=8.2 Hz, 2H), 7.66 (d, J=8.5 Hz, 2H), 7.48 (d, J=7.4 Hz, 2H), 7.34 (d, J=8.5 Hz, 2H), 7.26-7.08 (m, 2H), 6.96 (d, J=8.1 Hz, 1H), 4.82 (s, 2H), 4.61 (s, 2H), 4.54-4.26 (m, 2H), 3.87 (t, J=6.5 Hz, 1H), 2.96-2.85 (m, 2H), 2.82 (s, 1H), 2.03-1.75 (m, 2H), 1.66 (p, J=7.7 Hz, 2H), 1.41 (p, J=8.1 Hz, 2H), 0.84-0.60 (m, 2H), 0.60-0.39 (m, 2H). LCMS (ESI, +ve mode) expected m/z for [C₃₃H₃₉N₆O₅⁺] [M+H] 599.3, found 599.3.

### Example 107

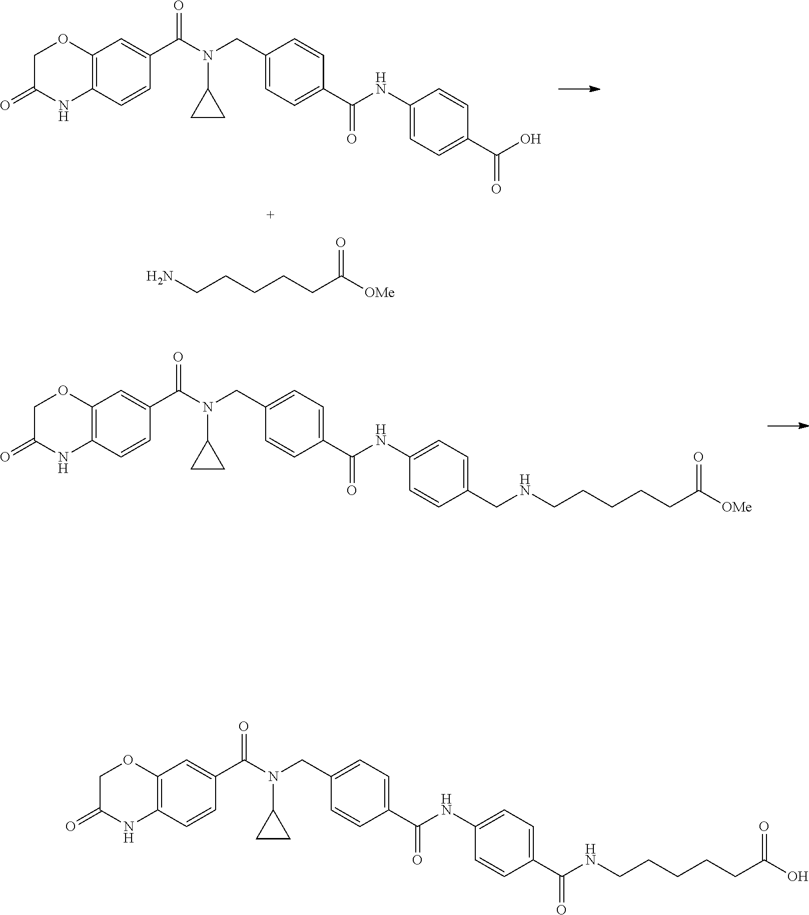 [SMILES: O=C1COc2cc(C(=O)N(Cc3ccc(C(=O)Nc4ccc(C(=O)O)cc4)cc3)C3CC3)ccc2N1 ; COC(=O)CCCCCN ; COC(=O)CCCCCNCc1ccc(NC(=O)c2ccc(CN(C(=O)c3ccc4c(c3)OCC(=O)N4)C3CC3)cc2)cc1 ; O=C(O)CCCCCNC(=O)c1ccc(NC(=O)c2ccc(CN(C(=O)c3ccc4c(c3)OCC(=O)N4)C3CC3)cc2)cc1]

Amide coupling performed according to General Procedure A/Workup A using 4-(4-((N-cyclopropyl-3-oxo-3,4-dihydro-2H-benzo[b][1,4]oxazine-7-carboxamido)methyl)benzamido) benzoic acid (50 mg, 1 Eq, 0.10 mmol), EDC (30 mg, 1.5 Eq, 0.15 mmol), HOAt (21 mg, 1.5 Eq, 0.15 mmol), DMF (1 mL), methyl 6-aminohexanoate hydrochloride (22 mg, 1.2 Eq, 0.12 mmol) and triethylamine (42 mg, 57 μL, 4 Eq, 0.41 mmol). The material was purified by normal phase chromatography over silica gel (0-10% MeOH in DCM) to give pure methyl 6-(4-(4-((N-cyclopropyl-3-oxo-3,4-dihydro-2H-benzo[b][1,4]oxazine-7-carboxamido)methyl)benzamido) benzamido)hexanoate. To the flask was added 1,4-Dioxane (16 mL) and lithium hydroxide hydrate (22 mg, 5 Eq, 0.51 mmol) in Water (4 mL). The mixture was stirred overnight. The still-basic aqueous layer was washed with ether 5 times. The solution was then acidifed to a pH of 2-3 with 2 M HCl. The acidic layer was extracted 3× with ethyl acetate. Combined ethyl acetate layers were washed with brine, dried over magnesium sulfate, filtered and concentrated in vacuo. The product was taken up in a minimal amount of MeOH/DCM and triturated into a large volume of ether with stirring. The precipitate was collected by filtration and washed with additional ether. The solid was recrystallized from a minimal amount of boiling ethanol to give 6-(4-(4-((N-cyclopropyl-3-oxo-3,4-dihydro-2H-benzo[b][1,4]oxazine-7-carboxamido)methyl)benzamido)benzamido)hexanoic acid (4.14 mg, 6.92 μmol, 6.7%) as a white solid. Characterization of ester: ¹H NMR (400 MHz, dmso) δ 10.87 (br s, 1H, NH), 10.42 (br s, 1H, NH), 8.35 (t, J=5.6 Hz, 1H, NH), 7.95 (d, J=8.2 Hz, 2H), 7.90-7.71 (m, 4H), 7.46 (d, J=8.1 Hz, 2H), 7.27-7.07 (m, 2H), 6.93 (d, J=8.1 Hz, 1H), 4.72 (s, 2H), 4.62 (s, 2H), 3.58 (s, 3H), 3.24 (q, J=6.5 Hz, 2H), 2.80 (s, 1H), 2.31 (t, J=7.4 Hz, 2H), 1.54 (hept, J=7.3 Hz, 4H), 1.31 (p, J=7.5 Hz, 2H), 0.76-0.51 (m, 2H), 0.52-0.27 (m, 2H). LCMS (ESI, +ve mode) expected m/z for [C₃₄H₃₇N₄O₇⁺] [M+H] 613.3, found 613.2.

Characterization of acid: ¹H NMR (400 MHz, dmso) δ 10.40 (br s, 1H, NH), 8.33 (t, J=5.6 Hz, 1H, NH), 7.94 (d, J=8.3 Hz, 2H), 7.89-7.73 (m, 4H), 7.44 (d, J=8.0 Hz, 2H), 7.23-7.06 (m, 2H), 6.91 (d, J=8.0 Hz, 1H), 4.70 (s, 2H), 4.60 (s, 2H), 3.22 (q, J=6.5 Hz, 2H), 2.78 (s, 1H), 2.19 (t, J=7.3 Hz, 2H), 1.59-1.42 (m, 4H), 1.39-1.24 (m, 3H), 0.60-0.50 (m, 2H), 0.50-0.32 (m, 2H). LCMS (ESI, +ve mode) expected m/z for [C₃₃H₃₅N₄O₇⁺] [M+H] 599.2, found 599.2.

### Example 108

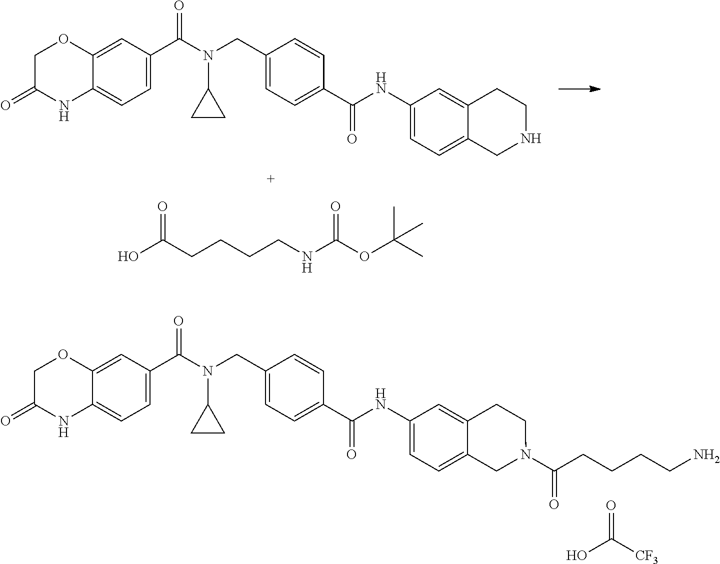

Prepared according to General Procedure A/Workup A using 5-((tert-butoxycarbonyl)amino)pentanoic acid (23 mg, 1 Eq, 0.11 mmol), EDC (41 mg, 2 Eq, 0.21 mmol), HOAt (29 mg, 2 Eq, 0.21 mmol), DMF (1.5 mL), N-cyclopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (71 mg, 1.1 Eq, 0.12 mmol) and triethylamine (43 mg, 59 μL, 4 Eq, 0.42 mmol). Following N-Boc deprotection, the product was purified by reverse phase chromatography (10-100% methanol in water+0.1% TFA) to give N-(4-((2-(5-aminopentanoyl)-1,2,3,4-tetrahydroisoquinolin-6-yl)carbamoyl)benzyl)-N-cyclopropyl-3-oxo-3,4-dihydro-2H-benzo[b][1,4]oxazine-7-carboxamide 2,2,2-trifluoroacetate (41.08 mg, 57.88 μmol, 55%) as a white solid. ¹H NMR (400 MHz, cd₃od) δ7.92 (d, J=8.2 Hz, 2H), 7.68-7.37 (m, 4H), 7.25-7.06 (m, 3H), 6.95 (d, J=8.1 Hz, 1H), 4.80 (s, 2H), 4.68 (d, J=4.5 Hz, 2H), 4.60 (s, 2H), 3.77 (dt, J=15.3, 6.0 Hz, 2H), 3.02-2.91 (m, 3H), 2.91-2.83 (m, 1H), 2.81 (s, 1H), 2.62-2.43 (m, 2H), 1.84-1.57 (m, 4H), 0.76-0.59 (m, 2H), 0.60-0.39 (m, 2H). LCMS (ESI, +ve mode) expected m/z for [C₃₄H₃₈N₅O₅⁺] [M+H] 596.3, found 596.2.

### Example 109

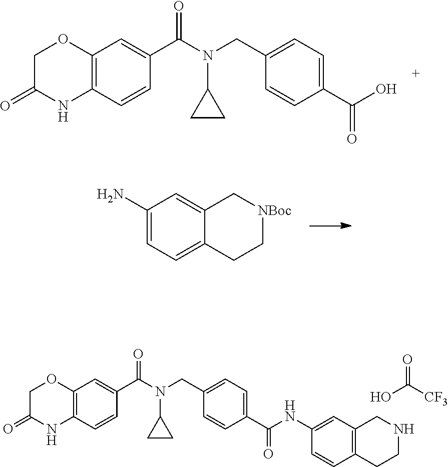

Prepared according to General Procedure A/Workup A using 4-((N-cyclopropyl-3-oxo-3,4-dihydro-2H-benzo[b][1,4]oxazine-7-carboxamido)methyl)benzoic acid (75 mg, 1 Eq, 0.20 mmol), EDC (59 mg, 1.5 Eq, 0.31 mmol), HOAt (42 mg, 1.5 Eq, 0.31 mmol), DMF (1.5 mL), tert-butyl 7-amino-3,4-dihydroisoquinoline-2(1H)-carboxylate (51 mg, 1 Eq, 0.20 mmol) and triethylamine (83 mg, 0.11 mL, 4 Eq, 0.82 mmol). Following N-Boc deprotection, the product was purified by reverse phase chromatography (10-100% methanol in water+0.1% TFA) to give N-cyclopropyl-3-oxo-N-(4-((1,2,3,4-tetrahydroisoquinolin-7-yl)carbamoyl)benzyl)-3,4-dihydro-2H-benzo[b][1,4]oxazine-7-carboxamide 2,2,2-trifluoroacetate (97.1 mg, 159 μmol, 78%) as a clear resin. ¹H NMR (400 MHz, cd₃od) δ 7.92 (d, J=8.2 Hz, 2H), 7.71-7.51 (m, 2H), 7.46 (d, J=7.5 Hz, 2H), 7.30-7.05 (m, 3H), 6.95 (d, J=8.1 Hz, 1H), 4.80 (s, 2H), 4.60 (s, 2H), 4.34 (s, 2H), 3.50 (t, J=6.4 Hz, 2H), 3.09 (t, J=6.3 Hz, 2H), 2.81 (s, 1H), 0.87-0.59 (m, 2H), 0.59-0.23 (m, 2H). LCMS (ESI, +ve mode) expected m/z for [C₂₉H₂₉N₄O₄⁺] [M+H] 497.2, found 497.2.

### Example 110

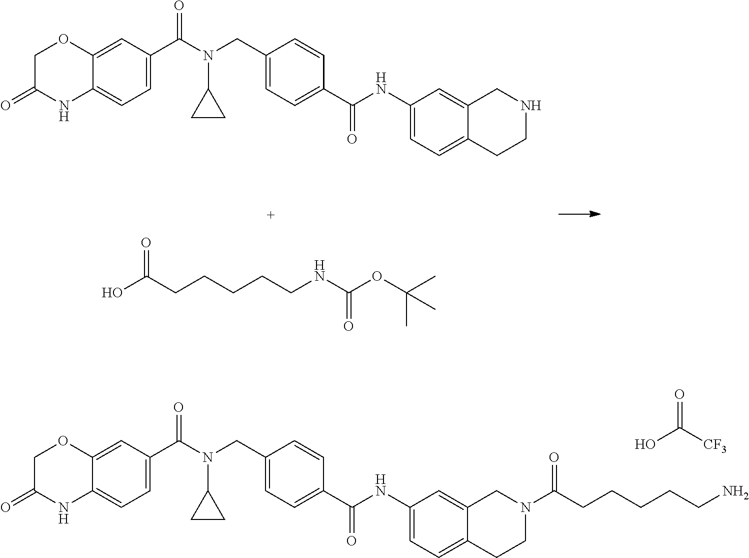 [SMILES: O=C1COc2cc(C(=O)N(Cc3ccc(C(=O)Nc4ccc5c(c4)CNCC5)cc3)C3CC3)ccc2N1 ; CC(C)(C)OC(=O)NCCCCCC(=O)O ; NCCCCCC(=O)N1CCc2ccc(NC(=O)c3ccc(CN(C(=O)c4ccc5c(c4)OCC(=O)N5)C4CC4)cc3)cc2C1 ; O=C(O)C(F)(F)F]

Prepared according to General Procedure A/Workup A using 6-((tert-butoxycarbonyl)amino)hexanoic acid (17 mg, 1 Eq, 74 μmol), EDC (28 mg, 2 Eq, 0.15 mmol), HOAt (20 mg, 2 Eq, 0.15 mmol), DMF (1.5 mL), N-cyclopropyl-3-oxo-N-(4-((1,2,3,4-tetrahydroisoquinolin-7-yl)carbamoyl)benzyl)-3,4-dihydro-2H-benzo[b][1,4]oxazine-7-carboxamide 2,2,2-trifluoroacetate (50 mg, 1.1 Eq, 82 μmol) and triethylamine (30 mg, 41 μL, 4 Eq, 0.30 mmol). Following N-Boc deprotection, the product was purified by reverse phase chromatography (10-100% methanol in water+0.1% TFA) to give N-(4-((2-(6-aminohexanoyl)-1,2,3,4-tetrahydroisoquinolin-7-yl)carbamoyl)benzyl)-N-cyclopropyl-3-oxo-3,4-dihydro-2H-benzo[b][1,4]oxazine-7-carboxamide 2,2,2-trifluoroacetate (45 mg, 62 μmol, 85%) as a white solid. ¹H NMR (400 MHz, cd₃od) δ 8.00-7.81 (m, 2H), 7.72-7.31 (m, 4H), 7.27-7.06 (m, 3H), 6.95 (d, J=8.0 Hz, 1H), 4.81 (s, 2H), 4.70 (d, J=3.4 Hz, 2H), 4.61 (s, 2H), 3.78 (dt, J=15.8, 6.0 Hz, 2H), 3.00-2.88 (m, 3H), 2.83 (t, J=5.8 Hz, 2H), 2.52 (q, J=7.1 Hz, 2H), 1.68 (h, J=7.4 Hz, 4H), 1.57-1.27 (m, 2H), 0.84-0.59 (m, 2H), 0.59-0.35 (m, 2H). LCMS (ESI, +ve mode) expected m/z for [C₃₅H₄₀N₅O₅⁺] [M+H] 610.3, found 610.3.

### Example 111

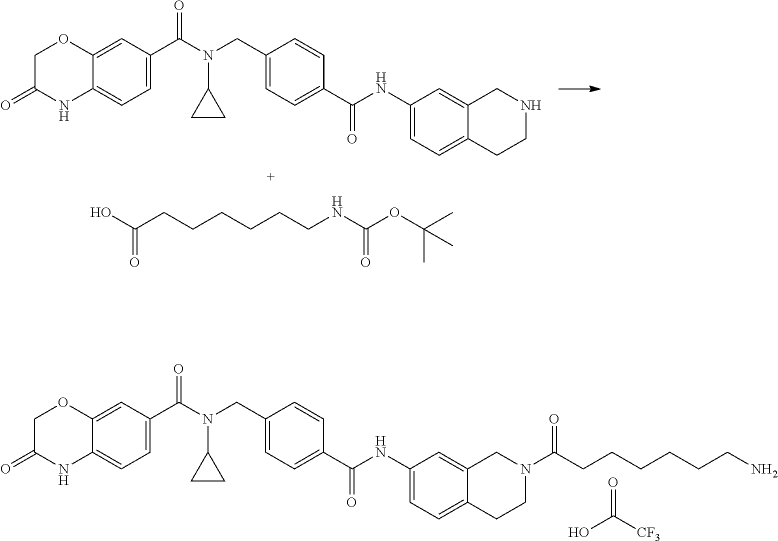

Prepared according to General Procedure A/Workup A using 7-((tert-butoxycarbonyl)amino)heptanoic acid (17 mg, 1 Eq, 70 μmol), EDC (27 mg, 2 Eq, 0.14 mmol), HOAt (19 mg, 2 Eq, 0.14 mmol), DMF (1.5 mL), N-cyclopropyl-3-oxo-N-(4-((1,2,3,4-tetrahydroisoquinolin-7-yl)carbamoyl)benzyl)-3,4-dihydro-2H-benzo[b][1,4]oxazine-7-carboxamide 2,2,2-trifluoroacetate (47 mg, 1.1 Eq, 77 μmol), triethylamine (28 mg, 39 μL, 4 Eq, 0.28 mmol). Following N-Boc deprotection, the product was purified by reverse phase chromatography (10-100% methanol in water+0.1% TFA) to give N-(4-((2-(7-aminoheptanoyl)-1,2,3,4-tetrahydroisoquinolin-7-yl)carbamoyl)benzyl)-N-cyclopropyl-3-oxo-3,4-dihydro-2H-benzo[b][1,4]oxazine-7-carboxamide 2,2,2-trifluoroacetate (29.88 mg, 40.50 μmol, 58%) as a white solid. ¹H NMR (400 MHz, cd₃od) δ8.01-7.85 (m, 2H), 7.70-7.33 (m, 4H), 7.27-7.05 (m, 3H), 6.95 (d, J=8.0 Hz, 1H), 4.81 (s, 2H), 4.70 (d, J=6.3 Hz, 2H), 4.61 (s, 2H), 3.78 (dt, J=14.1, 6.0 Hz, 2H), 2.99-2.86 (m, 3H), 2.83 (t, J=5.8 Hz, 2H), 2.51 (q, J=7.2 Hz, 2H), 1.80-1.51 (m, 4H), 1.51-1.28 (m, 4H), 0.89-0.61 (m, 2H), 0.59-0.25 (m, 2H). LCMS (ESI, +ve mode) expected m/z for [C₃₆H₄₂N₅O⁺] [M+H] 624.3, found 624.3.

### Example 112

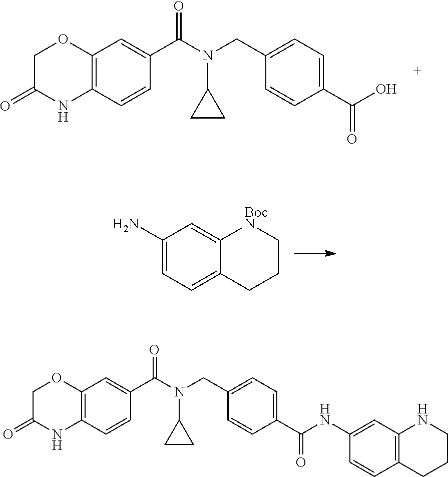 [SMILES: O=C1COc2cc(C(=O)N(Cc3ccc(C(=O)O)cc3)C3CC3)ccc2N1 ; CC(C)(C)OC(=O)N1CCCc2ccc(N)cc21 ; O=C1COc2cc(C(=O)N(Cc3ccc(C(=O)Nc4ccc5c(c4)NCCC5)cc3)C3CC3)ccc2N1]

Prepared according to General Procedure A/Workup A using 4-((N-cyclopropyl-3-oxo-3,4-dihydro-2H-benzo[b][1,4]oxazine-7-carboxamido)methyl)benzoic acid (75 mg, 1 Eq, 0.20 mmol), EDC (59 mg, 1.5 Eq, 0.31 mmol), HOAt (42 mg, 1.5 Eq, 0.31 mmol), DMF (1.5 mL), tert-butyl 7-amino-3,4-dihydroquinoline-1(2H)-carboxylate (51 mg, 1 Eq, 0.20 mmol) and triethylamine (83 mg 0.11 mL, 4 Eq, 0.82 mmol). Following N-Boc deprotection, the product was purified by reverse phase chromatography (10-100% methanol in water+0.1% TFA) to give N-cyclopropyl-3-oxo-N-(4-((1,2,3,4-tetrahydroquinolin-7-yl)carbamoyl)benzyl)-3,4-dihydro-2H-benzo[b][1,4]oxazine-7-carboxamide (98 mg, 0.20 mmol, 96%) as a clear resin ¹H NMR (400 MHz, cd₃od) δ 8.02-7.84 (m, 3H), 7.60-7.39 (m, 3H), 7.36-7.23 (m, 1H), 7.23-7.08 (m, 2H), 6.94 (d, J=8.1 Hz, 1H), 4.81 (s, 2H), 4.60 (s, 2H), 3.56-3.41 (m, 2H), 2.90 (t, J=6.4 Hz, 2H), 2.81 (s, 1H), 2.13 (p, J=6.3 Hz, 2H), 0.93-0.59 (m, 2H), 0.59-0.28 (m, 2H). LCMS (ESI, +ve mode) expected m/z for [C₂₉H₂₉N₄O₄⁺] [M+H] 497.2, found 497.2.

### Example 113

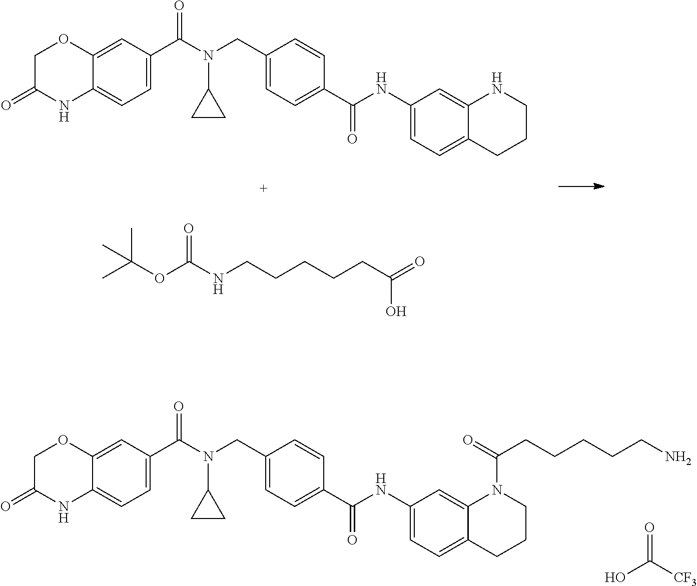 [SMILES: O=C1COc2cc(C(=O)N(Cc3ccc(C(=O)Nc4ccc5c(c4)NCCC5)cc3)C3CC3)ccc2N1 ; CC(C)(C)OC(=O)NCCCCCC(=O)O ; NCCCCCC(=O)N1CCCc2ccc(NC(=O)c3ccc(CN(C(=O)c4ccc5c(c4)OCC(=O)N5)C4CC4)cc3)cc21 ; O=C(O)C(F)(F)F]

Prepared according to General Procedure A/Workup A using 6-((tert-butoxycarbonyl)amino)hexanoic acid (22 mg, 1 Eq, 95 μmol), EDC (36 mg, 2 Eq, 0.19 mmol), HOAt (26 mg, 2 Eq, 0.19 mmol), DMF (1.5 mL), N-cyclopropyl-3-oxo-N-(4-((1,2,3,4-tetrahydroisoquinolin-7-yl)carbamoyl)benzyl)-3,4-dihydro-2H-benzo[b][1,4]oxazine-7-carboxamide 2,2,2-trifluoroacetate (64 mg, 1.1 Eq, 0.10 mmol) and triethylamine (39 mg, 53 μL, 4 Eq, 0.38 mmol). Following N-Boc deprotection, the product was purified by reverse phase chromatography (10-100% methanol in water+0.1% TFA) to give N-(4-((2-(6-aminohexanoyl)-1,2,3,4-tetrahydroisoquinolin-7-yl)carbamoyl)benzyl)-N-cyclopropyl-3-oxo-3,4-dihydro-2H-benzo[b][1,4]oxazine-7-carboxamide 2,2,2-trifluoroacetate (3.05 mg, 4.21 μmol, 5.1%) as a white solid. ¹H NMR (400 MHz, cd₃od) δ 7.93 (d, J=8.2 Hz, 3H), 7.49 (d, J=7.6 Hz, 2H), 7.36-7.08 (m, 4H), 6.96 (d, J=8.0 Hz, 1H), 4.83 (s, 3H), 4.62 (s, 2H), 3.79 (t, J=6.5 Hz, 2H), 2.91 (t, J=7.4 Hz, 2H), 2.84 (s, 1H), 2.74 (t, J=6.5 Hz, 2H), 2.67 (t, J=7.4 Hz, 2H), 1.97 (p, J=6.6 Hz, 2H), 1.69 (dp, J=22.7, 7.7 Hz, 4H), 1.52-1.30 (m, 2H), 0.87-0.60 (m, 2H), 0.61-0.38 (m, 2H). LCMS (ESI, +ve mode) expected m/z for [C₃₅H₄₀N₅O₅][M+H] 610.3, found 610.3.

### Example 114

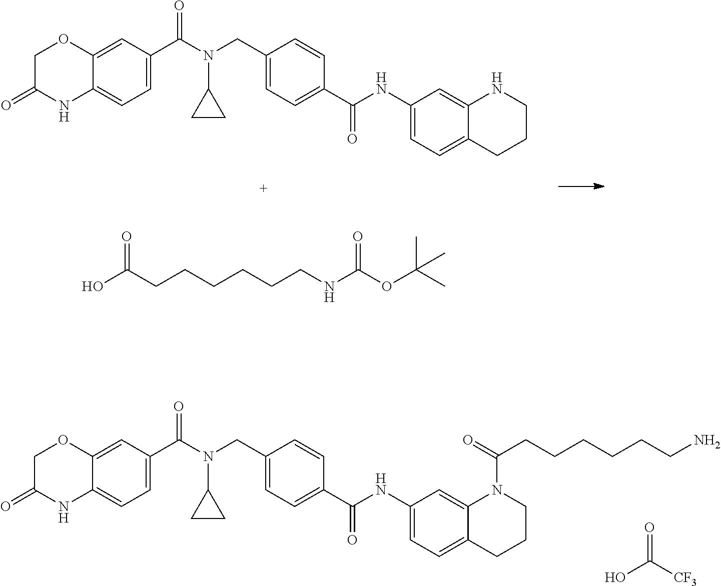 [SMILES: O=C1COc2cc(C(=O)N(Cc3ccc(C(=O)Nc4ccc5c(c4)NCCC5)cc3)C3CC3)ccc2N1 ; CC(C)(C)OC(=O)NCCCCCCC(=O)O ; NCCCCCCC(=O)N1CCCc2ccc(NC(=O)c3ccc(CN(C(=O)c4ccc5c(c4)OCC(=O)N5)C4CC4)cc3)cc21 ; O=C(O)C(F)(F)F]

Prepared according to General Procedure A/Workup A using 7-((tert-butoxycarbonyl)amino)heptanoic acid (22 mg, 1 Eq, 95 μmol), EDC (36 mg, 2 Eq, 0.19 mmol), HOAt (26 mg, 2 Eq, 0.19 mmol), DMF (1.5 mL), N-cyclopropyl-3-oxo-N-(4-((1,2,3,4-tetrahydroquinolin-7-yl)carbamoyl)benzyl)-3,4-dihydro-2H-benzo[b][1,4]oxazine-7-carboxamide 2,2,2-trifluoroacetate (64 mg, 1.1 Eq, 0.10 mmol) and triethylamine (39 mg, 53 μL, 4 Eq, 0.38 mmol). Following N-Boc deprotection, the product was purified by reverse phase chromatography (10-100% methanol in water+0.1% TFA) to give N-(4-((1-(7-aminoheptanoyl)-1,2,3,4-tetrahydroquinolin-7-yl)carbamoyl)benzyl)-N-cyclopropyl-3-oxo-3,4-dihydro-2H-benzo[b][1,4]oxazine-7-carboxamide 2,2,2-trifluoroacetate (2.97 mg, 4.03 μmol, 4.5%) as a white solid. ¹H NMR (400 MHz, cd₃od) δ 8.06-7.81 (m, 3H), 7.49 (d, J=7.5 Hz, 2H), 7.41-7.09 (m, 4H), 6.96 (d, J=8.1 Hz, 1H), 4.82 (s, 2H), 4.62 (s, 2H), 3.79 (t, J=6.5 Hz, 2H), 2.94-2.78 (m, 3H), 2.73 (t, J=6.6 Hz, 2H), 2.65 (t, J=7.4 Hz, 2H), 2.06-1.90 (m, 2H), 1.79-1.48 (m, 4H), 1.47-1.19 (m, 4H), 0.84-0.60 (m, 2H), 0.60-0.39 (m, 2H). LCMS (ESI, +ve mode) expected m/z for [C₃₆H₄₂N₅O₅] [M+H] 624.3, found 624.3.

### Example 115

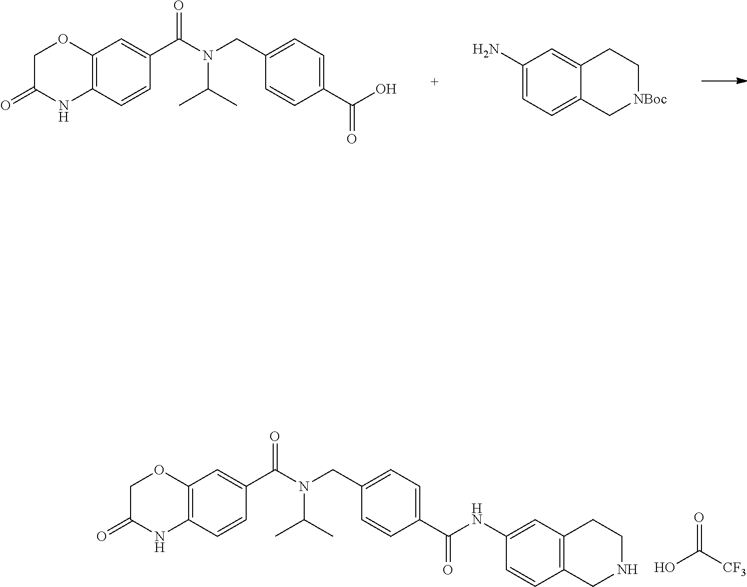 [SMILES: CC(C)N(Cc1ccc(C(=O)O)cc1)C(=O)c1ccc2c(c1)OCC(=O)N2 ; CC(C)(C)OC(=O)N1CCc2cc(N)ccc2C1 ; CC(C)N(Cc1ccc(C(=O)Nc2ccc3c(c2)CCNC3)cc1)C(=O)c1ccc2c(c1)OCC(=O)N2 ; O=C(O)C(F)(F)F]

Prepared according to General Procedure A/Workup A using 4-((N-isopropyl-3-oxo-3,4-dihydro-2H-benzo[b][1,4]oxazine-7-carboxamido)methyl)benzoic acid (50 mg, 1 Eq, 0.14 mmol), EDC (39 mg, 1.5 Eq, 0.20 mmol), HOAt (28 mg, 1.5 Eq, 0.20 mmol), DMF (1.5 mL), tert-butyl 6-amino-3,4-dihydroisoquinoline-2(1H)-carboxylate (34 mg, 1 Eq, 0.14 mmol) and triethylamine (55 mg, 75 μL, 4 Eq, 0.54 mmol). Following N-Boc deprotection, the product was purified by reverse phase chromatography (10-100% methanol in water+0.1% TFA) to give N-isopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (73 mg, 0.12 mmol, 88%) as a clear resin. ¹H NMR (400 MHz, cd₃od) δ 7.90 (d, J=8.1 Hz, 2H), 7.66 (s, 1H), 7.57 (dd, J=8.4, 2.0 Hz, 1H), 7.53-7.27 (m, 2H), 7.19 (d, J=8.5 Hz, 1H), 7.15-6.73 (m, 3H), 4.72 (s, 2H), 4.61 (s, 2H), 4.32 (s, 2H), 4.23 (s, 1H), 3.49 (t, J=6.4 Hz, 2H), 3.11 (t, J=6.3 Hz, 2H), 1.48-0.76 (m, 6H). LCMS (ESI, +ve mode) expected m/z for [C₂₉H₃₁N₄O₄⁺] [M+H] 499.2, found 499.2.

### Example 116

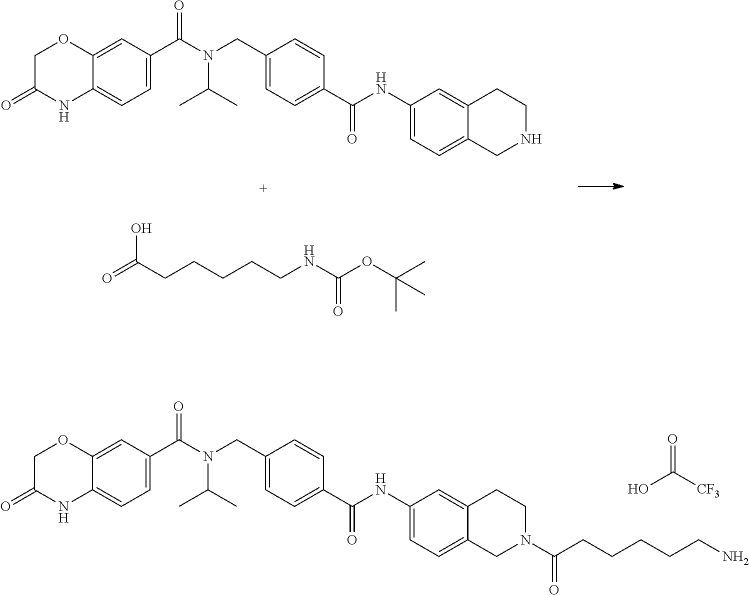 [SMILES: CC(C)N(Cc1ccc(C(=O)Nc2ccc3c(c2)CCNC3)cc1)C(=O)c1ccc2c(c1)OCC(=O)N2 ; CC(C)(C)OC(=O)NCCCCCC(=O)O ; CC(C)N(Cc1ccc(C(=O)Nc2ccc3c(c2)CCN(C(=O)CCCCCN)C3)cc1)C(=O)c1ccc2c(c1)OCC(=O)N2 ; O=C(O)C(F)(F)F]

Prepared according to General Procedure A/Workup A using 6-((tert-butoxycarbonyl)amino)hexanoic acid (15 mg, 1 Eq, 65 μmol), EDC (46 mg, 2 Eq, 0.24 mmol), HOAt (32 mg, 2 Eq, 0.24 mmol), DMF (1 mL), N-isopropyl-3-oxo-N-(4-((1,2,3,4-tetrahydroisoquinolin-6-yl)carbamoyl)benzyl)-3,4-dihydro-2H-benzo[b][1,4]oxazine-7-carboxamide 2,2,2-trifluoroacetate (73 mg, 1 Eq, 0.12 mmol) and triethylamine (48 mg, 66 μL, 4 Eq, 0.48 mmol). Following N-Boc deprotection, the product was purified by reverse phase chromatography (10-100% methanol in water+0.1% TFA) to give N-(4-((2-(6-aminohexanoyl)-1,2,3,4-tetrahydroisoquinolin-6-yl)carbamoyl)benzyl)-N-isopropyl-3-oxo-3,4-dihydro-2H-benzo[b][1,4]oxazine-7-carboxamide 2,2,2-trifluoroacetate (2.84 mg, 3.91 μmol, 6.0%) as a white solid. ¹H NMR (400 MHz, cd₃od) δ 7.89 (d, J=8.1 Hz, 2H), 7.63-7.20 (m, 4H), 7.19-7.11 (m, 1H), 7.11-6.67 (m, 3H), 4.72 (s, 2H), 4.67 (d, J=4.8 Hz, 2H), 4.61 (s, 2H), 4.23 (s, 1H), 3.76 (dt, J=15.4, 6.0 Hz, 2H), 2.97-2.87 (m, 3H), 2.83 (t, J=5.9 Hz, 1H), 2.51 (td, J=7.3, 3.8 Hz, 2H), 1.67 (h, J=7.6 Hz, 4H), 1.43 (h, J=7.4 Hz, 2H), 1.35-0.90 (m, 6H). LCMS (ESI, +ve mode) expected m/z for [C₃₅H₄₂N₅O₅⁺] [M+H] 612.3, found 612.3.

### Example 117

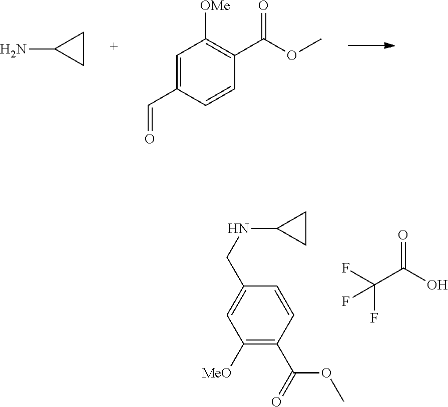

To a scintillation vial equipped with a stir bar was added methyl 4-formyl-2-methoxybenzoate (347.1 mg, 1 Eq, 1.787 mmol) and methanol (2 mL). The flask was sealed with a septum, and cyclopropanamine (204.1 mg, 0.25 mL, 2 Eq, 3.575 mmol) was added via syringe. The flask was capped and stirred at room temperature overnight to ensure imine formation. The flask was cooled in an ice/water bath and sodium tetrahydroborate (135.2 mg, 2 Eq, 3.575 mmol) was added portionwise. The reaction was allowed to come to room temperature overnight. The reaction was quenched by addition of saturated sodium bicarbonate, and extracted three times with ethyl acetate. The combined organic layers were washed once more with saturated sodium bicarbonate, once with brine, then dried over sodium sulfate and concentrated to an oil. Normal phase chromatography on silica (0-100% EtOAc in hexanes) provided the free base as a yellow free-flowing oil. The oil was dissolved in 3 mL of diethyl ether and cooled in an ice bath, and TFA (346.5 mg, 234 μL, 1.7 Eq, 3.039 mmol) was added dropwise. A voluminous white solid formed, which was filtered and washed rigorously with diethyl ether to provide methyl 4-((cyclopropylamino)methyl)-2-methoxybenzoate 2,2,2-trifluoroacetate (250.6 mg, 717.4 μmol, 40.14%) as a fluffy white powder. ¹H NMR (400 MHz, cd₃od) δ 7.71 (d, J=7.9 Hz, 1H), 7.11 (s, 1H), 6.96 (dd, J=7.9, 1.2 Hz, 1H), 4.83 (br s, 1H, NH), 3.87 (s, 3H), 3.83 (s, 3H), 3.81 (s, 2H), 2.24-1.97 (m, 1H), 0.53-0.42 (m, 2H), 0.42-0.23 (m, 2H). LCMS (ESI, +ve mode) expected m/z for [C₁₃H₁₈NO₃⁺] [M+H] 236.1, found 236.2.

### Example 118

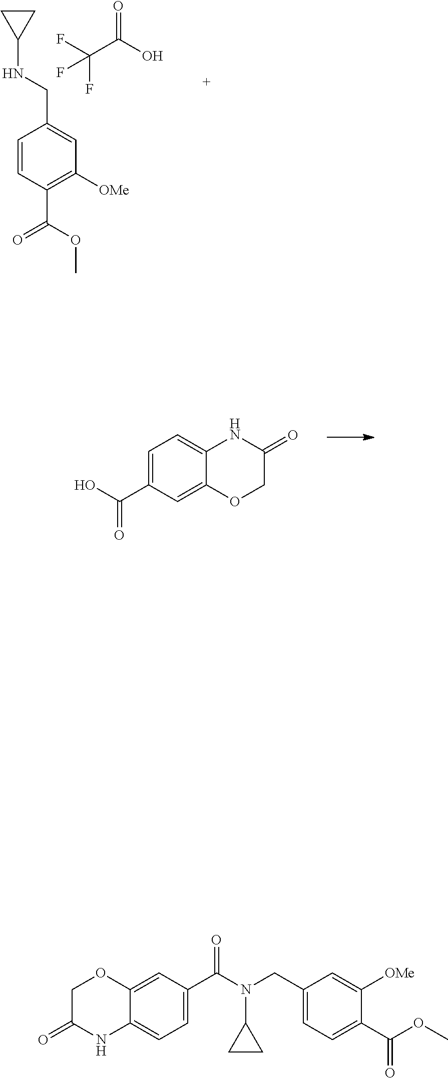

Prepared according to General Procedure A/Workup A using methyl 3-oxo-3,4-dihydro-2H-benzo[b][1,4]oxazine-7-carboxylate (138.6 mg, 1 Eq, 717.4 μmol), EDC (206.3 mg, 1.5 Eq, 1.076 mmol), HOAt(146.5 mg, 1.5 Eq, 1.076 mmol), MeCN (4 mL), methyl 4-((cyclopropylamino)methyl)-2-methoxybenzoate 2,2,2-trifluoroacetate (250.6 mg, 1 Eq, 717.4 μmol) and triethylamine (239.6 mg 0.33 mL, 3.3 Eq, 2.367 mmol). Following workup, the crude residue was purified by normal phase chromatography on silica gel (0-100% ethyl acetate in hexanes) to give methyl 4-((N-cyclopropyl-3-oxo-3,4-dihydro-2H-benzo[b][1,4]oxazine-7-carboxamido)methyl)-2-methoxybenzoate (248.5 mg, 605.5 μmol, 84.40%) as a white solid. ¹H NMR (400 MHz, cd₃od) δ 7.75 (d, J=7.9 Hz, 1H), 7.28-7.09 (m, 2H), 7.04 (s, 1H), 7.00-6.82 (m, 2H), 4.76 (s, 2H), 4.60 (s, 2H), 3.87 (s, 3H), 3.84 (s, 3H), 2.81 (s, 1H), 0.81-0.58 (m, 2H), 0.59-0.35 (m, 2H). LCMS (ESI, +ve mode) expected m/z for [C₂₂H₂₃N₂O₆⁺] [M+H] 411.2, found 411.1.

### Example 119

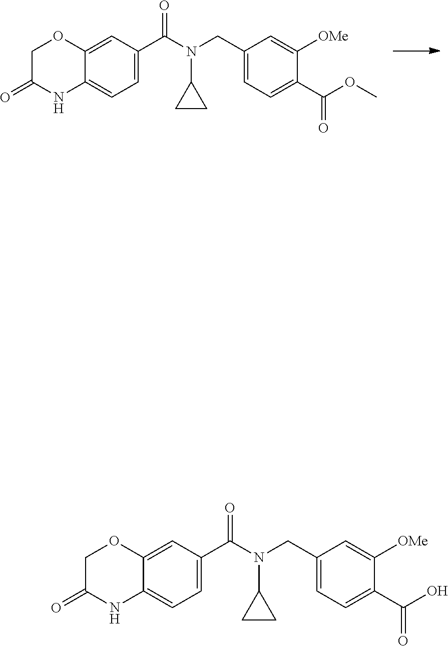 [SMILES: COC(=O)c1ccc(CN(C(=O)c2ccc3c(c2)OCC(=O)N3)C2CC2)cc1OC ; COc1cc(CN(C(=O)c2ccc3c(c2)OCC(=O)N3)C2CC2)ccc1C(=O)O]

Prepared according to General Procedure D using methyl 4-((N-cyclopropyl-3-oxo-3,4-dihydro-2H-benzo[b][1,4]oxazine-7-carboxamido)methyl)-2-methoxybenzoate (248.5 mg, 1 Eq, 605.5 μmol), dioxane (6 mL), lithium hydroxide hydrate (127.0 mg, 5 Eq, 3.027 mmol) and water (2 mL) to give 4-((N-cyclopropyl-3-oxo-3,4-dihydro-2H-benzo[b][1,4]oxazine-7-carboxamido)methyl)-2-methoxybenzoic acid (148.2 mg, 373.9 μmol, 61.75%) as a dry, white powder.

### Example 120

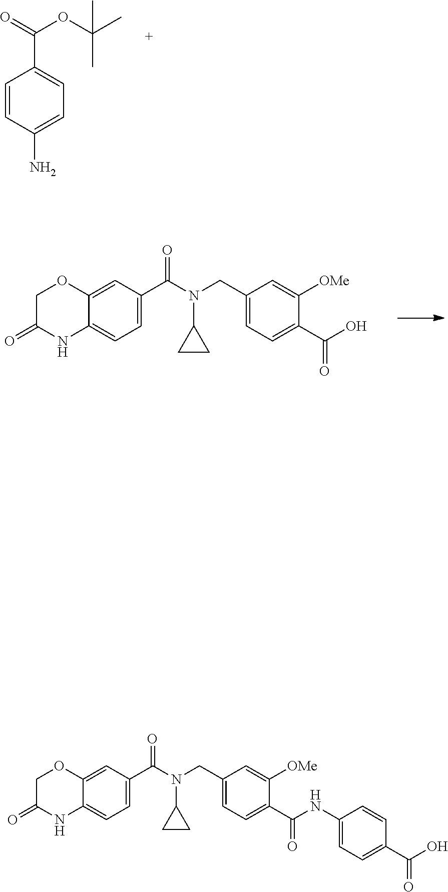 [SMILES: CC(C)(C)OC(=O)c1ccc(N)cc1 ; COc1cc(CN(C(=O)c2ccc3c(c2)OCC(=O)N3)C2CC2)ccc1C(=O)O ; COc1cc(CN(C(=O)c2ccc3c(c2)OCC(=O)N3)C2CC2)ccc1C(=O)Nc1ccc(C(=O)O)cc1]

Prepared and purified according to General Procedure G using 4-((N-cyclopropyl-3-oxo-3,4-dihydro-2H-benzo[b][1,4]oxazine-7-carboxamido)methyl)-2-methoxybenzoic acid (100 mg, 1 Eq, 252 μmol), EDC (96.7 mg, 2 Eq, 505 μmol), DMAP (61.6 mg, 2 Eq, 505 μmol), DMF (4 mL) and tert-butyl 4-aminobenzoate (97.5 mg, 2 Eq, 505 μmol) at 50° C. The following day, the reaction was cooled to room temperature and diluted with ethyl acetate and water. The phases were separated and the aqueous layer extracted twice more with ethyl acetate. The organic layer was washed once with 1M KHSO₄, once with water, once with saturated sodium bicarbonate, and once with brine, then dried over sodium sulfate and concentrated in vacuo. Normal phase chromatography over silica gel (0-10% methanol in DCM) followed by N-Boc deprotection gave 4-(4-((N-cyclopropyl-3-oxo-3,4-dihydro-2H-benzo[b][1,4]oxazine-7-carboxamido)methyl)-2-methoxybenzamido)benzoic acid (39.8 mg, 77.2 μmol, 30.6%) as a clear resin. ¹H NMR (400 MHz, cd₃od) δ 8.08-7.85 (m, 3H), 7.78 (d, J=8.7 Hz, 2H), 7.32-7.00 (m, 4H), 6.96 (d, J=8.0 Hz, 1H), 4.80 (s, 2H), 4.62 (s, 2H), 4.04 (s, 3H), 2.85 (s, 1H), 1.59 (s, 9H), 0.75-0.60 (m, 2H), 0.61-0.35 (m, 2H). LCMS (ESI, +ve mode) expected m/z for [C₂₈H₂₆N₃O₇⁺] [M+H] 516.2, found 516.1.

### Example 121

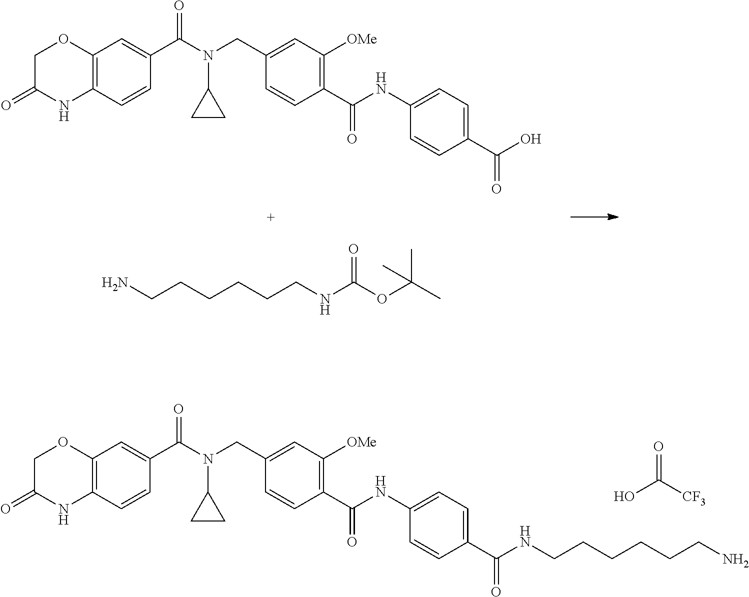 [SMILES: COc1cc(CN(C(=O)c2ccc3c(c2)OCC(=O)N3)C2CC2)ccc1C(=O)Nc1ccc(C(=O)O)cc1 ; CC(C)(C)OC(=O)NCCCCCCN ; COc1cc(CN(C(=O)c2ccc3c(c2)OCC(=O)N3)C2CC2)ccc1C(=O)Nc1ccc(C(=O)NCCCCCCN)cc1 ; O=C(O)C(F)(F)F]

Prepared according to General Procedure A/Workup A using 4-(4-((N-cyclopropyl-3-oxo-3,4-dihydro-2H-benzo[b][1,4]oxazine-7-carboxamido)methyl)-2-methoxybenzamido)benzoic acid (39.8 mg, 1 Eq, 77.2 μmol), EDC (22.2 mg, 1.5 Eq, 116 μmol), HOAt (15.8 mg, 1.5 Eq, 116 μmol), DMF (1 mL), tert-butyl (6-aminohexyl)carbamate (25.1 mg, 1.5 Eq, 116 μmol) and triethylamine (31.2 mg 43 μL, 4 Eq, 309 μmol). Following N-Boc deprotection, the product was purified by reverse phase chromatography (10-100% methanol in water+0.1% TFA) to give N-(4-((4-((6-aminohexyl)carbamoyl)phenyl)carbamoyl)-3-methoxybenzyl)-N-cyclopropyl-3-oxo-3,4-dihydro-2H-benzo[b][1,4]oxazine-7-carboxamide 2,2,2-trifluoroacetate (2.29 mg, 3.15 μmol, 5.20%) as a white solid. ¹H NMR (400 MHz, cd₃od) δ 7.92 (d, J=7.9 Hz, 1H), 7.88-7.68 (m, 4H), 7.32-7.03 (m, 4H), 6.97 (d, J=8.0 Hz, 1H), 4.82 (s, 2H), 4.63 (s, 2H), 4.05 (s, 3H), 3.40 (t, J=7.1 Hz, 2H), 2.93 (t, J=7.5 Hz, 2H), 2.86 (s, 1H), 1.79-1.56 (m, 4H), 1.56-1.38 (m, 4H), 0.82-0.62 (m, 2H), 0.62-0.35 (m, 2H). LCMS (ESI, +ve mode) expected m/z for [C₃₄H₄₀N₅O₆⁺] [M+H] 614.3, found 614.2.

### Example 122

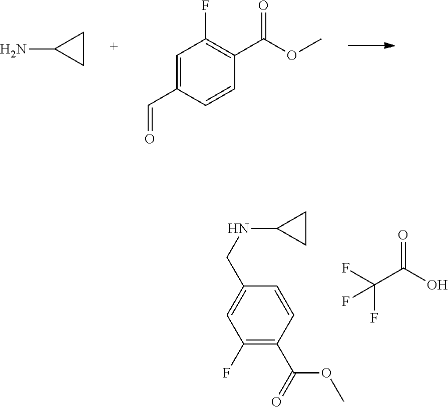

Prepared and purified according to general procedure X using methyl 2-fluoro-4-formylbenzoate (500 mg, 1 Eq, 2.74 mmol), methanol (6 mL), cyclopropanamine (313 mg, 0.39 mL, 2 Eq, 5.49 mmol) and sodium tetrahydroborate (208 mg, 2 Eq, 5.49 mmol) to methyl 4-((cyclopropylamino)methyl)-2-fluorobenzoate 2,2,2-trifluoroacetate (356.4 mg, 1.057 mmol, 38.5%) as a fluffy white powder. ¹H NMR (400 MHz, cdcl₃) δ 7.91 (t, J=7.7 Hz, 1H), 7.37-7.11 (m, 2H), 3.93 (s, 3H, NH), 3.88 (s, 2H), 3.34 (p, J=1.6 Hz, 1H), 2.22-2.09 (m, 1H), 0.67-0.45 (m, 2H), 0.45-0.16 (m, 2H). LCMS (ESI, +ve mode) expected m/z for [C₁₂H₁₅FNO₂⁺] [M+H] 224.1, found 224.1.

### Example 123

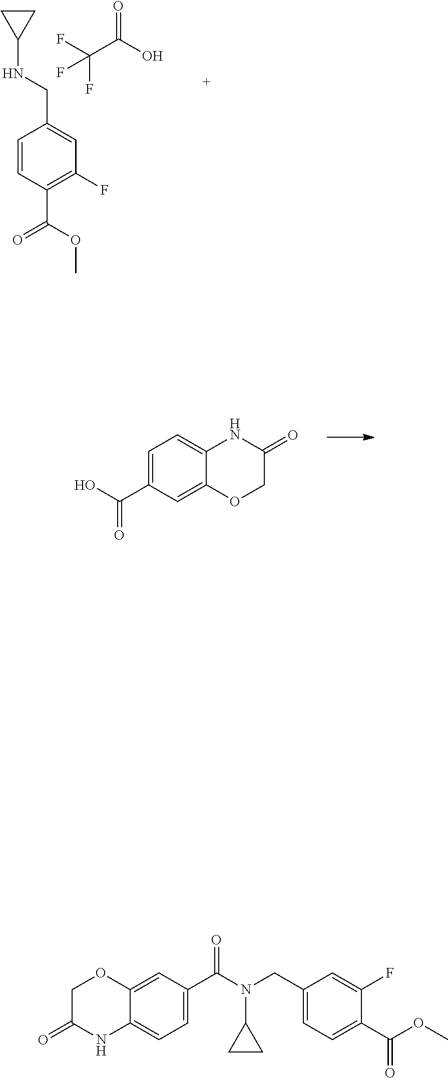

Prepared and purified according to General Procedure A/Workup A using methyl 3-oxo-3,4-dihydro-2H-benzo[b][1,4]oxazine-7-carboxylate (204.1 mg, 1 Eq, 1.057 mmol), EDC (303.9 mg, 1.5 Eq, 1.585 mmol), HOAt (215.7 mg, 1.5 Eq, 1.585 mmol), MeCN (3 mL), methyl 4-((cyclopropylamino)methyl)-2-fluorobenzoate 2,2,2-trifluoroacetate (356.4 mg, 1 Eq, 1.057 mmol), and triethylamine (352.9 mg, 0.48 mL, 3.3 Eq, 3.487 mmol). Following workup, the crude material was purified by normal phase chromatography over silica gel (0-100% EtOAc in hexanes) to give methyl 4-((N-cyclopropyl-3-oxo-3,4-dihydro-2H-benzo[b][1,4]oxazine-7-carboxamido)methyl)-2-fluorobenzoate (288.9 mg, 725.2 μmol, 68.62%) as a clear resin. ¹H NMR (400 MHz, cd₃od) δ 7.79-7.46 (m, 1H), 7.11-6.78 (m, 4H), 6.78-6.47 (m, 1H), 4.51 (s, 2H), 4.35 (s, 2H), 3.63 (s, 3H), 2.59 (s, 1H), 0.73-0.34 (m, 2H), 0.33-−0.01 (m, 2H). LCMS (ESI, +ve mode) expected m/z for [C₂₁H₂₀FN₂O₅⁺] [M+H] 399.1, found 399.1.

### Example 124

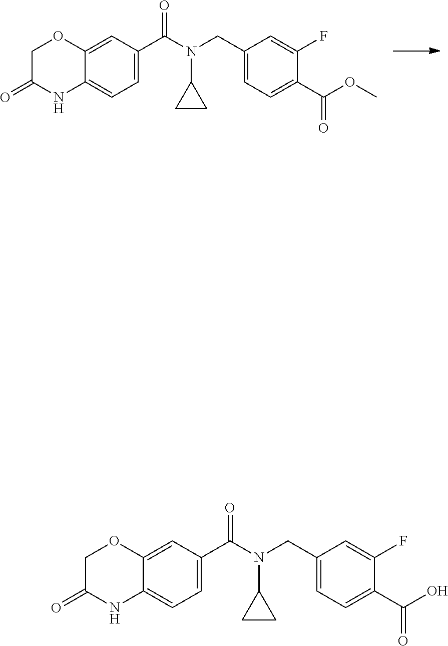 [SMILES: COC(=O)c1ccc(CN(C(=O)c2ccc3c(c2)OCC(=O)N3)C2CC2)cc1F ; O=C1COc2cc(C(=O)N(Cc3ccc(C(=O)O)c(F)c3)C3CC3)ccc2N1]

Prepared according to General Procedure D using methyl 4-((N-cyclopropyl-3-oxo-3,4-dihydro-2H-benzo[b][1,4]oxazine-7-carboxamido)methyl)-2-fluorobenzoate (288.9 mg, 1 Eq, 725.2 μmol), dioxane (6 mL), lithium hydroxide hydrate (152.1 mg, 5 Eq, 3.626 mmol) and water (2 mL) to give 4-((N-cyclopropyl-3-oxo-3,4-dihydro-2H-benzo[b][1,4]oxazine-7-carboxamido)methyl)-2-fluorobenzoic acid (221.9 mg, 577.3 μmol, 79.61%) as a dry, white powder. ¹H NMR (400 MHz, dmso) δ 13.15 (br s, 1H, OH), 10.85 (s, 1H, NH), 7.84 (s, 1H), 7.51-7.02 (m, 4H), 6.90 (d, J=6.9 Hz, 1H), 4.66 (s, 2H), 4.60 (s, 2H), 2.85 (s, 1H), 0.77-0.47 (m, 2H), 0.48-−0.40 (m, 2H).

### Example 125

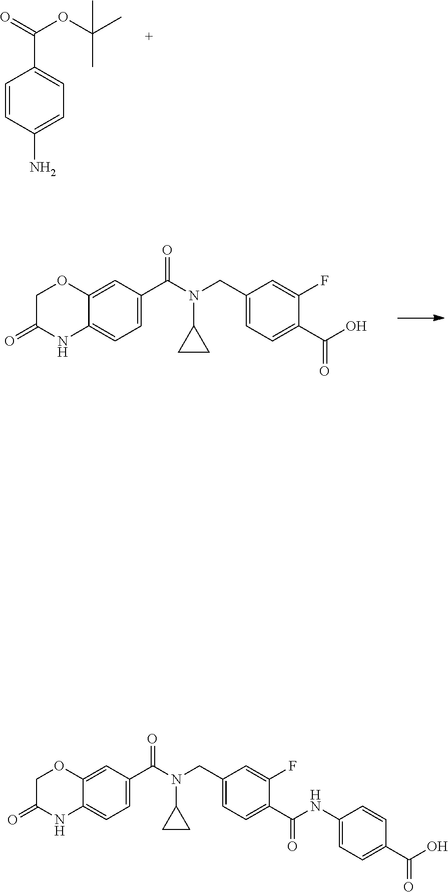 [SMILES: CC(C)(C)OC(=O)c1ccc(N)cc1 ; O=C1COc2cc(C(=O)N(Cc3ccc(C(=O)O)c(F)c3)C3CC3)ccc2N1 ; O=C1COc2cc(C(=O)N(Cc3ccc(C(=O)Nc4ccc(C(=O)O)cc4)c(F)c3)C3CC3)ccc2N1]

Prepared according to General Procedure G using 4-((N-cyclopropyl-3-oxo-3,4-dihydro-2H-benzo[b][1,4]oxazine-7-carboxamido)methyl)-2-fluorobenzoic acid (221.9 mg, 1 Eq, 577.3 μmol), EDC (221.3 mg, 2 Eq, 1.155 mmol), DMAP (141.1 mg, 2 Eq, 1.155 mmol), DMF (4 mL) and tert-butyl 4-aminobenzoate (223.1 mg, 2 Eq, 1.155 mmol). The following day, the reaction was cooled to room temperature and diluted with ethyl acetate and water. The phases were separated and the aqueous layer extracted twice more with ethyl acetate. The organic layer was washed once with 1M KHSO₄, once with water, once with saturated sodium bicarbonate, and once with brine, then dried over sodium sulfate and concentrated in vacuo. Normal phase chromatography over silica gel (0-10% methanol in DCM) followed by N-Boc deprotection gave 4-(4-((N-cyclopropyl-3-oxo-3,4-dihydro-2H-benzo[b][1,4]oxazine-7-carboxamido)methyl)-2-fluorobenzamido)benzoic acid (25.4 mg, 50.4 μmol, 8.74%) as a clear resin. ¹H NMR (400 MHz, cd₃od) δ 7.96-7.89 (m, 2H), 7.85-7.68 (m, 3H), 7.38-7.12 (m, 4H), 6.96 (d, J=8.0 Hz, 1H), 4.80 (s, 2H), 4.62 (s, 2H), 2.89 (s, 1H), 0.74-0.60 (m, 2H), 0.60-0.35 (m, 2H). LCMS (ESI, +ve mode) expected m/z for [C₂₇H₂₃FN₃O₆⁺] [M+H] 504.2, found 504.1.

### Example 126

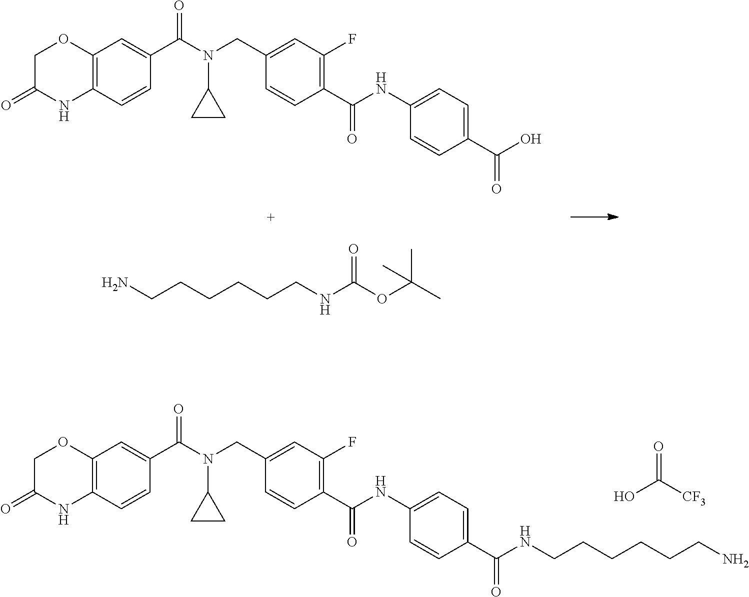 [SMILES: O=C1COc2cc(C(=O)N(Cc3ccc(C(=O)Nc4ccc(C(=O)O)cc4)c(F)c3)C3CC3)ccc2N1 ; CC(C)(C)OC(=O)NCCCCCCN ; NCCCCCCNC(=O)c1ccc(NC(=O)c2ccc(CN(C(=O)c3ccc4c(c3)OCC(=O)N4)C3CC3)cc2F)cc1 ; O=C(O)C(F)(F)F]

Prepared according to General Procedure A/Workup A using 4-(4-((N-cyclopropyl-3-oxo-3,4-dihydro-2H-benzo[b][1,4]oxazine-7-carboxamido)methyl)-2-fluorobenzamido)benzoic acid (25.4 mg, 1 Eq, 50.4 μmol), EDC (14.5 mg, 1.5 Eq, 75.7 μmol), HOAt (10.3 mg, 1.5 Eq, 75.7 μmol), DMF (1 mL), tert-butyl (6-aminohexyl)carbamate (16.4 mg, 1.5 Eq, 75.7 μmol) and triethylamine (20.4 mg 28 μL, 4 Eq, 202 μmol). Following N-Boc deprotection, the product was purified by reverse phase chromatography (10-100% methanol in water+0.1% TFA) to give N-(4-((4-((6-aminohexyl)carbamoyl)phenyl)carbamoyl)-3-fluorobenzyl)-N-cyclopropyl-3-oxo-3,4-dihydro-2H-benzo[b][1,4]oxazine-7-carboxamide 2,2,2-trifluoroacetate (3.10 mg, 4.33 μmol, 8.59%) as a white solid. ¹H NMR (400 MHz, cd₃od) δ 7.89-7.77 (m, 4H), 7.76 (t, J=7.6 Hz, 1H), 7.38-7.10 (m, 4H), 6.97 (d, J=8.1 Hz, 1H), 4.81 (s, 2H), 4.63 (s, 2H), 3.40 (t, J=7.1 Hz, 2H), 3.01-2.89 (m, 3H), 2.87 (s, 1H), 1.77-1.57 (m, 4H), 1.57-1.37 (m, 4H), 0.82-0.62 (m, 2H), 0.61-0.33 (m, 2H). LCMS (ESI, +ve mode) expected m/z for [C₃₃H₃₇FN₅O₅⁺] [M+H] 602.3, found 602.3.

### Example 127

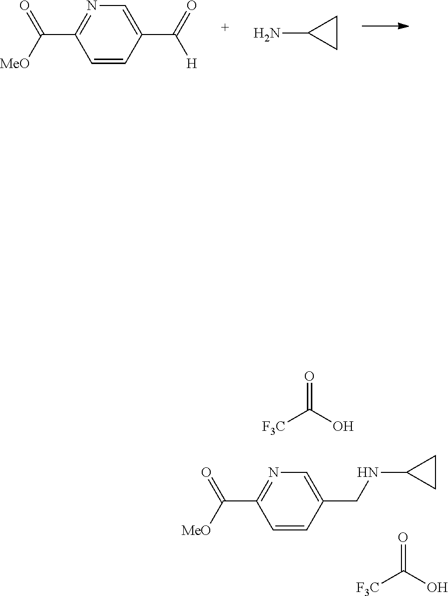

Prepared according to general procedure X using methyl 5-formylpicolinate (500 mg, 1 Eq, 3.03 mmol), methanol, cyclopropylamine (207 mg, 0.26 mL, 1.2 Eq, 3.63 mmol), acetic acid (364 mg 346 μL, 2 Eq, 6.06 mmol) and sodium cyanoborohydride (761 mg, 4 Eq, 12.1 mmol). The reaction was quenched by addition of water and washed three times with ethyl acetate (organic layers discarded). Sodium bicarbonate was then added and the mixture was extracted three more times with ethyl acetate. Combined organics were washed with brine, dried over magnesium sulfate, filtered and concentrated in vacuo. The oil was dissolved in 10 mL of diethyl ether and cooled in an ice bath, and 2,2,2-trifluoroacetic acid (587 mg, 397 μL, 1.7 Eq, 5.15 mmol) was added dropwise. A voluminous white solid formed, which was filtered and washed rigorously with diethyl ether to provide methyl 5-((cyclopropylamino)methyl)picolinate bis(2,2,2-trifluoroacetate) (273.1 mg, 628.8 μmol, 20.8%) as a white powder. ¹H NMR (400 MHz, cdcl₃) δ 8.54 (s, 1H), 7.93 (d, J=8.0 Hz, 1H), 7.72 (d, J=8.0 Hz, 1H), 3.85 (s, 3H), 3.82 (s, 2H), 2.74 (s, 1H, NH), 2.03 (hept, J=3.7 Hz, 1H), 0.48-0.27 (m, 2H), 0.27-−0.07 (m, 2H).

### Example 128

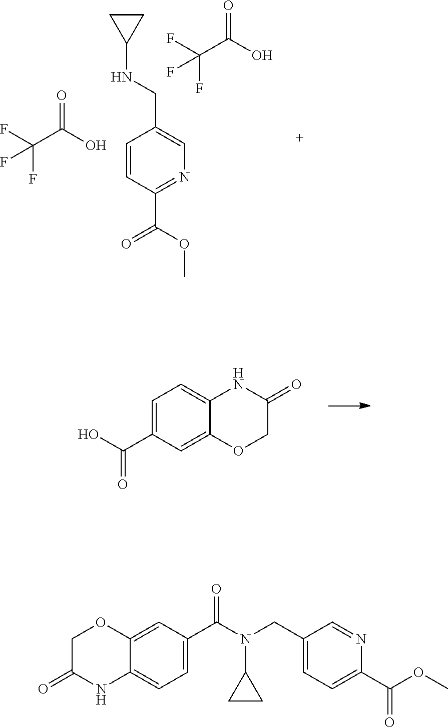

Prepared and purified according to General Procedure A/Workup A using methyl 3-oxo-3,4-dihydro-2H-benzo[b][1,4]oxazine-7-carboxylate (121.7 mg, 1 Eq, 630.0 μmol), EDC (181.2 mg, 1.5 Eq, 945.0 μmol), HOAt (128.6 mg, 1.5 Eq, 945.0 μmol), MeCN (3 mL), methyl 5-((cyclopropylamino)methyl)picolinate bis(2,2,2-trifluoroacetate) (273.6 mg, 1 Eq, 630.0 μmol) and triethylamine (210.4 mg, 0.29 mL, 3.3 Eq, 2.079 mmol). Following workup, the crude material was purified by normal phase chromatography over silica gel (0-100% EtOAc in hexanes) to give methyl 5-((N-cyclopropyl-3-oxo-3,4-dihydro-2H-benzo[b][1,4]oxazine-7-carboxamido)methyl)picolinate (207.9 mg, 545.1 μmol, 86.53%) as a white solid. ¹H NMR (400 MHz, cdcl₃) δ 10.00 (s, 1H, NH), 8.67 (s, 1H), 8.06 (d, J=8.0 Hz, 1H), 7.80 (d, J=8.0 Hz, 1H), 7.15-6.99 (m, 2H), 6.84 (d, J=8.0 Hz, 1H), 4.76 (s, 2H), 4.55 (s, 2H), 3.93 (s, 3H), 2.69-2.55 (m, 1H), 0.65-0.51 (m, 2H), 0.51-0.39 (m, 2H). LCMS (ESI, +ve mode) expected m/z for [C₂₀H₂₀N₃O₅⁺] [M+H] 382.1, found 382.1.

### Example 129

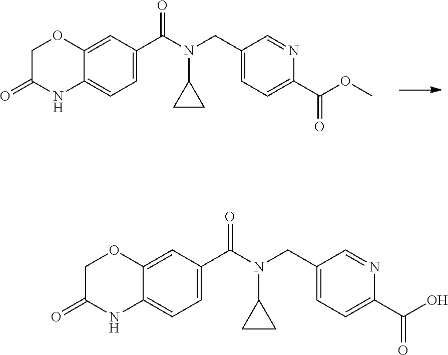 [SMILES: COC(=O)c1ccc(CN(C(=O)c2ccc3c(c2)OCC(=O)N3)C2CC2)cn1 ; O=C1COc2cc(C(=O)N(Cc3ccc(C(=O)O)nc3)C3CC3)ccc2N1]

Prepared according to General Procedure D using methyl 5-((N-cyclopropyl-3-oxo-3,4-dihydro-2H-benzo[b][1,4]oxazine-7-carboxamido)methyl)picolinate (207.9 mg, 1 Eq, 545.1 μmol), dioxane (4.5 mL), lithium hydroxide hydrate (114.4 mg, 5 Eq, 2.726 mmol) and water (1.5 mL). After washing the still-basic aqueous layer 5 times with ether, volatiles were removed in vacuo and the product was purified by reverse phase chromatography (10-100% methanol in water+0.1% TFA) to give 5-((N-cyclopropyl-3-oxo-3,4-dihydro-2H-benzo[b][1,4]oxazine-7-carboxamido)methyl)picolinic acid (123.8 mg, 337.0 μmol, 61.820%) as a white solid. ¹H NMR (400 MHz, cd₃od) δ 8.70 (s, 1H), 8.24 (d, J=8.1 Hz, 1H), 8.12 (d, J=8.3 Hz, 1H), 7.41-7.11 (m, 2H), 6.95 (d, J=7.9 Hz, 1H), 4.89 (s, 2H), 4.62 (s, 2H), 2.93 (s, 1H), 0.95-0.62 (m, 2H), 0.62-0.35 (m, 2H). LCMS (ESI, +ve mode) expected m/z for [C₁₉H₁₈N₃O₅⁺] [M+H] 368.1, found 368.1.

### Example 130

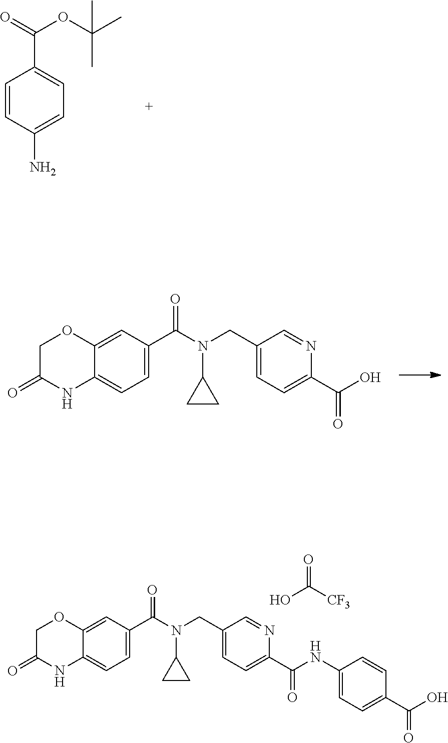

Prepared according to General Procedure G using 5-((N-cyclopropyl-3-oxo-3,4-dihydro-2H-benzo[b][1,4]oxazine-7-carboxamido)methyl)picolinic acid (32.5 mg, 1 Eq, 88.5 μmol), EDC (33.9 mg, 2 Eq, 177 μmol), DMAP (21.6 mg, 2 Eq, 177 μmol), DMF (2 mL) and tert-butyl 4-aminobenzoate (68.4 mg, 4 Eq, 354 μmol). The following day, the reaction was cooled to room temperature and diluted with ethyl acetate and water. The phases were separated and the aqueous layer extracted twice more with ethyl acetate. The organic layer was washed once with 1M KHSO₄, once with water, once with saturated sodium bicarbonate, and once with brine, then dried over sodium sulfate and concentrated in vacuo. Normal phase chromatography over silica gel (0-10% methanol in DCM) followed by t-Butyl ester cleavage gave 2,2,2-trifluoroacetic acid-4-(5-((N-cyclopropyl-3-oxo-3,4-dihydro-2H-benzo[b][1,4]oxazine-7-carboxamido)methyl)picolinamido)benzoic acid (1/1) (16 mg, 27 μmol, 30%) as a clear resin.

NMR of ^{t}Butyl-ester intermediate: ¹H NMR (400 MHz, cd₃od) δ 8.71 (s, 1H), 8.23 (d, J=8.1 Hz, 1H), 8.07-7.87 (m, 5H), 7.28-7.13 (m, 2H), 6.96 (d, J=8.0 Hz, 1H), 4.88 (s, 2H), 4.62 (s, 2H), 2.89 (s, 1H), 1.60 (s, 9H), 0.79-0.62 (m, 2H), 0.63-0.39 (m, 2H). LCMS (ESI, +ve mode) expected m/z for [C₂₆H₂₃N₄O₆⁺] [M+H] 487.2, found 487.1.

### Example 131

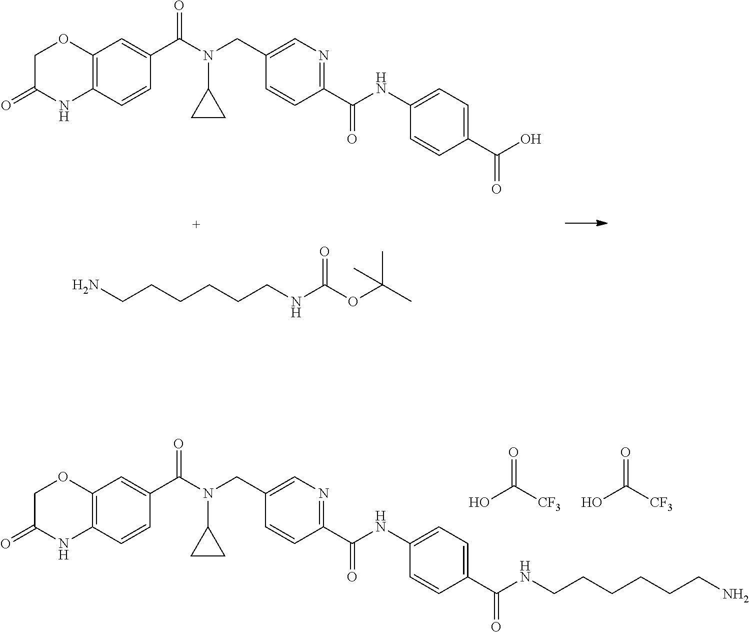

Prepared according to General Procedure A/Workup A as 4-(5-((N-cyclopropyl-3-oxo-3,4-dihydro-2H-benzo[b][1,4]oxazine-7-carboxamido)methyl)picolinamido)benzoic acid (16.00 mg, 1 Eq, 32.89 μmol), EDC (9.457 mg, 1.5 Eq, 49.33 μmol), HOAt (6.715 mg, 1.5 Eq, 49.33 μmol), DMF (1 mL), tert-butyl (6-aminohexyl)carbamate (10.67 mg, 1.5 Eq, 49.33 μmol) and triethylamine (13.31 mg, 18 μL, 4 Eq, 131.6 μmol). Following N-Boc deprotection, the product was purified by reverse phase chromatography (10-100% methanol in water+0.1% TFA) to give N-((6-((4-((6-aminohexyl)carbamoyl)phenyl)carbamoyl)pyridin-3-yl)methyl)-N-cyclopropyl-3-oxo-3,4-dihydro-2H-benzo[b][1,4]oxazine-7-carboxamide 2,2,2-trifluoroacetate (1.09 mg, 1.56 μmol, 5.4%) as a white solid. ¹H NMR (400 MHz, cd₃od) δ 8.72 (s, 1H), 8.23 (d, J=8.0 Hz, 1H), 8.02 (d, J=6.8 Hz, 1H), 7.97-7.80 (m, 4H), 7.27-7.12 (m, 2H), 6.96 (d, J=8.0 Hz, 1H), 4.88 (s, 2H), 4.62 (s, 2H), 3.40 (t, J=7.0 Hz, 2H), 3.00-2.87 (m, 3H), 1.78-1.56 (m, 4H), 1.56-1.37 (m, 4H), 0.81-0.63 (m, 2H), 0.63-0.39 (m, 2H). LCMS (ESI, +ve mode) expected m/z for [C₃₂H₃₇N₆O₅⁺] [M+H] 585.3, found 585.3.

### Example 132

### General Schemes

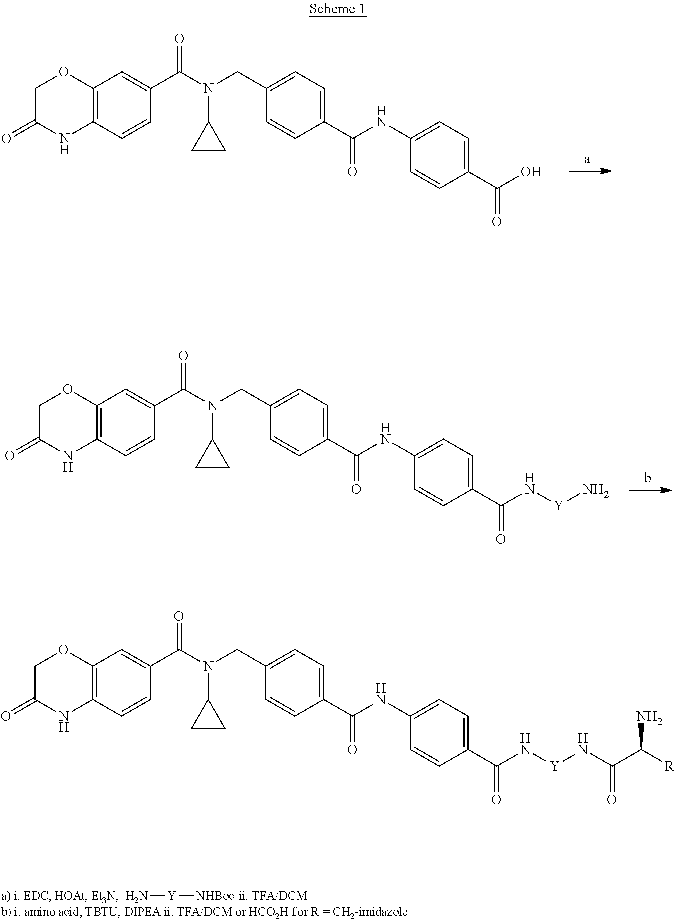

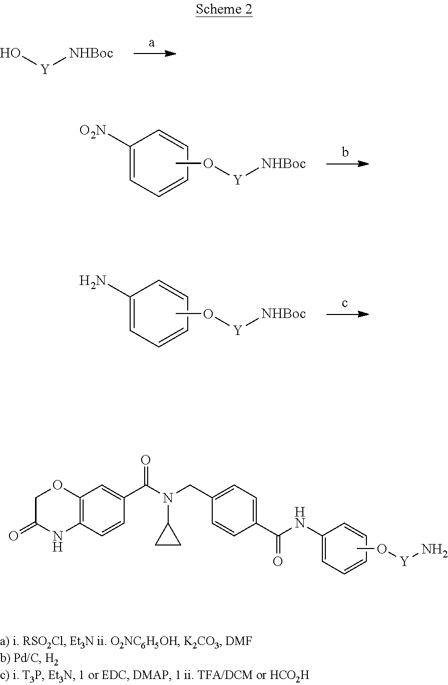

### General Procedures for Scheme 1 and Scheme 2:

### General Procedure I—Diamine Linker Addition/Deprotection:

To a flask were charged the starting acid (1 eq), EDC (1.5-2 eq), HOAt (1.5-2 eq), and DMF (0.4M). The reaction was stirred for 30 minutes to preactivate the acid. Next, the Boc-protected diamine (1.5-2 eq) was added neat (for solids) or as a solution in DMF (for liquids) alongside triethylamine (3 eq for free base amines; 4 eq for salts). The reaction was stirred at room temperature overnight.

The next day, the reaction was diluted with distilled water, and the white precipitate formed was isolated by filtration or by centrifugation for 5 minutes. The precipitate was washed again with water, air-dried, then taken up in ethanol, transferred to a flask, and evaporated to dryness. To the residue was then added 20% TFA in DCM, and the reaction was stirred until LCMS indicated consumption of starting material. The volatiles were removed on the rotovap, the residue co-evaporated with methanol to remove residual TFA, and purified by reverse phase chromatography.

### General Procedure II—Arginine Amidation/Deprotection:

To a vial were charged Boc3-Arg-OH (1.1 eq), TBTU (1.3 eq), and DMF (0.4M). The reaction was stirred for 10 minutes, then the amine (1 eq) was added, followed by DIPEA (3.3 eq). The reaction was stirred at room temperature overnight.

The next day, the reaction was diluted with water and extracted 3 times with ethyl acetate. The combined organic extracts were washed twice with water, once with saturated sodium bicarbonate, and once with brine, then dried by passage through a phase separator and concentrated to a residue. If starting amine was detected by LCMS of the crude, normal phase chromatography over silica gel (0-10% methanol in DCM) was performed.

To the Boc-protected product was added 50% TFA in DCM, and the reaction was stirred at room temperature overnight. The next day, the volatiles were removed on the rotovap, the residue co-evaporated with methanol to remove residual TFA, and purified by reverse phase chromatography.

### Histidine Amidation Deprotection:

To a vial were charged Boc-(Trt)His-OH (1.1 eq), TBTU (1.3 eq), and DMF (0.4M). The reaction was stirred for 10 minutes, then the amine (1 eq) was added, followed by DIPEA (3.3 eq).

The reaction was stirred at room temperature overnight.

The next day, the reaction was diluted with water and extracted 3 times with ethyl acetate. The combined organic extracts were washed twice with water, once with saturated sodium bicarbonate, and once with brine, then dried by passage through a phase separator and concentrated to a residue.

Purification of the tritylated histidine is inadvisable as the product streaks on silica.

To the protected product was added neat formic acid, and the reaction was stirred at room temperature overnight. The next day, a drop of methanol was added and the volatiles were removed on the rotovap, and the residue co-evaporated with methanol to remove residual formic acid. The residue was taken up in methanol/water, filtered, and purified by reverse phase chromatography.

### Other Amino Acid Amidation Deprotection:

To a vial were charged the Boc-protected amino acid (1.1 eq), TBTU (1.3 eq), and DMF (0.4M). The reaction was stirred for 10 minutes, then the amine (1 eq) was added, followed by DIPEA (3.3 eq). The reaction was stirred at room temperature overnight.

The next day, the reaction was diluted with water and extracted 3 times with ethyl acetate. The combined organic extracts were washed twice with water, once with saturated sodium bicarbonate, and once with brine, then dried by passage through a phase separator and concentrated to a residue.

To the protected product was added 20% TFA in DCM, and the reaction was stirred until LCMS indicated consumption of starting material. The volatiles were removed on the rotovap, the residue co-evaporated with methanol to remove residual TFA, and purified by reverse phase chromatography.

### Guanidinylation of Boc-Protected Amino Acid:

Sonogashira method: To a microwave vial or pressure tube equipped with stirbar were charged the aryl halide (1 eq), triphenylphosphine (0.1 eq), bis(triphenylphosphine)palladium dichloride (0.05 eq), cuprous iodide (0.1 eq), and, if a solid, the alkyne (1.5-2 eq). The vessel was sealed and the atmosphere cycled to nitrogen 3 times. Next, anhydrous dioxane (16 volumes) was added, and the solution sparged with nitrogen through a needle for 20 minutes. If a liquid, the alkyne (1.5 eq) was then added. The sparge tube was removed, anhydrous triethylamine (4 volumes) added, and the vessel crimped shut and heated to 100° C. for 16 hours. The reaction was cooled to room temperature, and the reaction quenched with saturated ammonium chloride and extracted 3 times with ethyl acetate. The combined organic extracts were washed once more with saturated ammonium chloride, once with water, once with saturated sodium bicarbonate, and once with brine, then dried over sodium sulfate and concentrated to a residue. The residue was purified by normal phase chromatography over silica gel.

### Alcohol Mesylation and Alkylation of Phenols:

To a flask equipped with stirbar were charged the alcohol (1 eq), triethylamine (1.8 eq), and dichloromethane (0.2 M). The flask was cooled in an ice bath, and mesyl chloride (1.4 eq) was added dropwise. The reaction was allowed to come to room temperature with stirring overnight. The next day, the reaction was quenched with 1M hydrochloric acid and extracted 3 times with dichloromethane. The combined organic extracts were washed once with water and once with brine, then dried over sodium sulfate and concentrated to an oil whose structure was verified by ¹H NMR.

To the crude mesylate were added the phenol (1.2 eq), potassium carbonate (1.5 eq), and DMF (0.4M). The reaction was capped and heated to 70° C. overnight. The next day, the reaction was diluted with water and extracted 3 times with ethyl acetate. The combined organic layers were washed three times with water and once with brine, then dried over sodium sulfate and concentrated to a residue. Normal phase chromatography over silica gel furnished the desired product. In cases where the phenol coelutes with the product, the crude residue is dissolved in dichloromethane and filtered through basic alumina prior to silica gel chromatography.

### Example 133

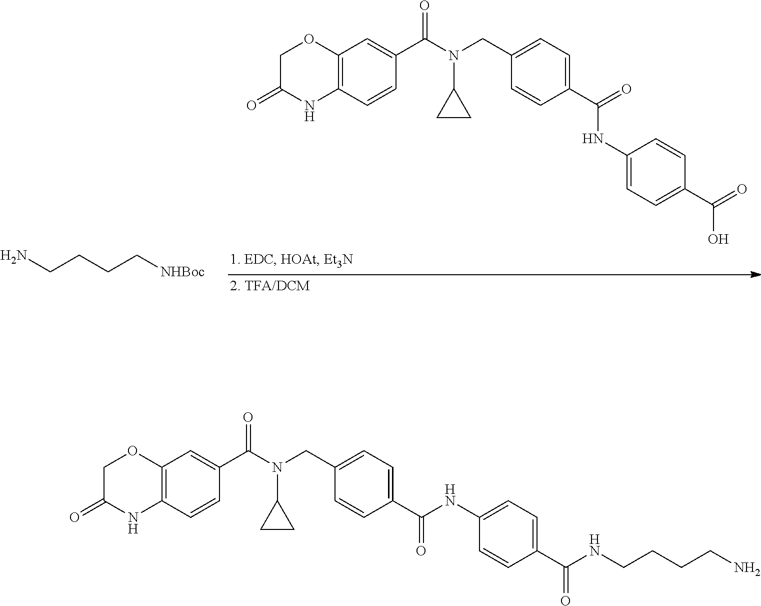 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCCCN ; NCCCCNC(=O)c1ccc(NC(=O)c2ccc(CN(C(=O)c3ccc4c(c3)OCC(=O)N4)C3CC3)cc2)cc1]

To a 2-dram vial were added 4-(4-((N-cyclopropyl-3-oxo-3,4-dihydro-2H-benzo[b][1,4]oxazine-7-carboxamido)methyl)benzamido)benzoic acid (22 mg, 1 Eq, 45 μmol), EDC (15 mg, 1.7 Eq, 78 μmol), HOAt (13 mg, 2.1 Eq, 96 μmol), and DMF (0.2 mL). The reaction was left to stir at room temperature for 30 minutes. To the vial was then added Carbamic acid, (4-aminobutyl)-, 1,1-dimethylethyl ester (15 mg, 1.8 Eq, 80 μmol) dissolved in DMF (0.2 mL), followed by triethylamine (16 mg, 22 μL, 3.5 Eq, 0.16 mmol), and the reaction was left to stir overnight.

The next day, the reaction was diluted with water and the white precipitate formed was isolated by centrifugation, washed once with water, taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (25.1 mg, 37.5 μmol, 83%) as a white solid. ¹H NMR (400 MHz, Methanol-d₄) δ 7.96 (d, J=8.2 Hz, 2H), 7.89-7.81 (m, 4H), 7.51 (d, J=7.9 Hz, 2H), 7.20 (d, J=8.5 Hz, 1H), 7.18 (s, 1H), 6.97 (d, J=8.0 Hz, 1H), 4.83 (s, 2H), 4.63 (s, 2H), 3.47-3.40 (m, 2H), 3.03-2.95 (m, 2H), 2.89-2.79 (m, 1H), 1.78-1.66 (m, 4H), 0.70-0.61 (m, 2H), 0.61-0.51 (m, 2H). LCMS (ESI, +ve mode) expected m/z for [M+H] 556.26, found 556.20.

### Example 134

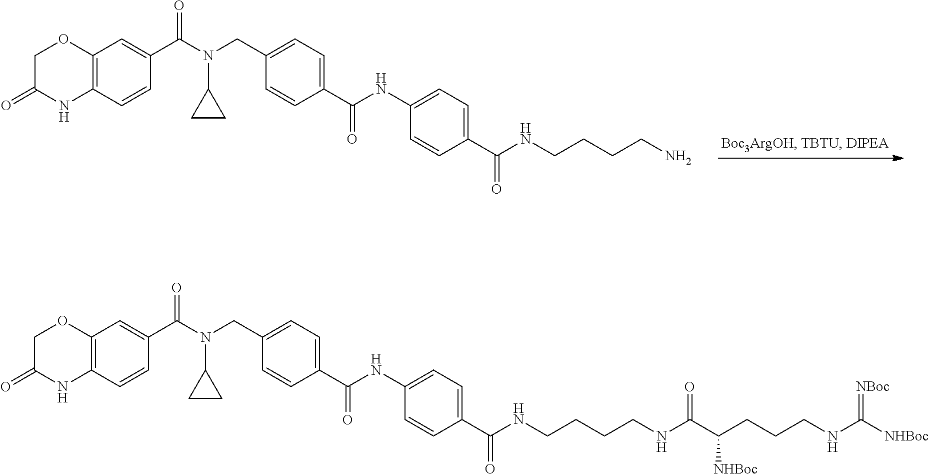

To a 2-dram vial were added (Z)—N2,Nw,Nw′-tris(tert-butoxycarbonyl)-L-arginine (12 mg, 1.1 Eq, 25 μmol), 2-(1H-benzo[d][1,2,3]triazol-1-yl)-1,1,3,3-tetramethylisouronium tetrafluoroborate (9.3 mg, 1.3 Eq, 29 μmol), DIPEA (9.6 mg, 13 μL, 3.3 Eq, 74 μmol), and DMF (0.5 mL). The reaction was left to stir at room temperature for 15 minutes. To the vial was then added N-(4-((4-((4-aminobutyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (15 mg, 1 Eq, 22 μmol) dissolved in DMF (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (17.2 mg, 17.0 μmol, 76%) as a clear gum. ¹H NMR (400 MHz, Chloroform-d) δ 9.34 (s, 2H), 9.14 (s, 1H), 7.89 (d, J=8.0 Hz, 2H), 7.79-7.70 (m, 4H), 7.32 (d, J=7.9 Hz, 2H), 7.16-7.11 (m, 1H), 7.11-7.01 (m, 3H), 6.85 (d, J=8.0 Hz, 1H), 5.99 (d, J=8.7 Hz, 1H), 4.74 (s, 2H), 4.58 (s, 2H), 4.25-4.16 (m, 1H), 3.95-3.84 (m, 1H), 3.76-3.66 (m, 1H), 3.46-3.36 (m, 2H), 3.36-3.26 (m, 1H), 3.25-3.17 (m, 1H), 2.69-2.61 (m, 1H), 1.73-1.54 (m, 8H), 1.49 (s, 9H), 1.47 (s, 9H), 1.42 (s, 9H), 0.65-0.55 (m, 2H), 0.54-0.43 (m, 2H). LCMS (ESI, +ve mode) expected m/z for [M+H]⁺ 1012.51, found 1012.40.

### Example 135

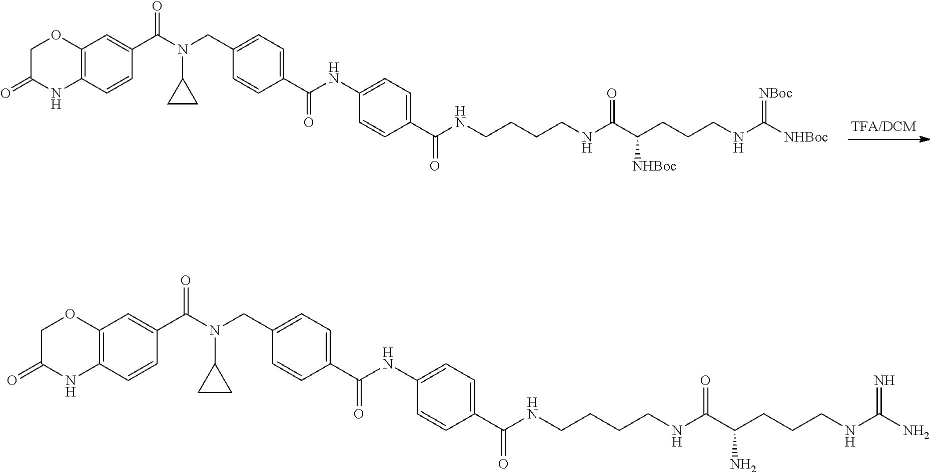

To the Reactant (12.58 mg, 1 Eq, 12.43 μmol) was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((4-((4-(2-amino-5-guanidinopentanamido)butyl)carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate) (7.02 mg, 7.47 μmol, 60.1%) as a white solid. ¹H NMR (400 MHz, Methanol-d₄) δ 7.96 (d, J=8.3 Hz, 2H), 7.88-7.81 (m, 4H), 7.50 (d, J=8.0 Hz, 2H), 7.20 (d, J=8.5 Hz, 1H), 7.17 (s, 1H), 6.97 (d, J=8.0 Hz, 1H), 4.83 (s, 2H), 4.62 (s, 2H), 3.83 (t, J=6.5 Hz, 1H), 3.41 (t, J=6.5 Hz, 2H), 3.38-3.34 (m, 1H), 3.30-3.26 (m, 1H), 3.23 (t, J=7.1 Hz, 2H), 2.89-2.79 (m, 1H), 1.97-1.82 (m, 2H), 1.74-1.59 (m, 6H), 0.71-0.61 (m, 2H), 0.61-0.51 (m, 2H). LCMS (ESI, +ve mode) expected m/z for [M+H]⁺ 712.36, found 712.30; expected m/z for [M+2H]²+356.68, found 356.70.

### Example 136

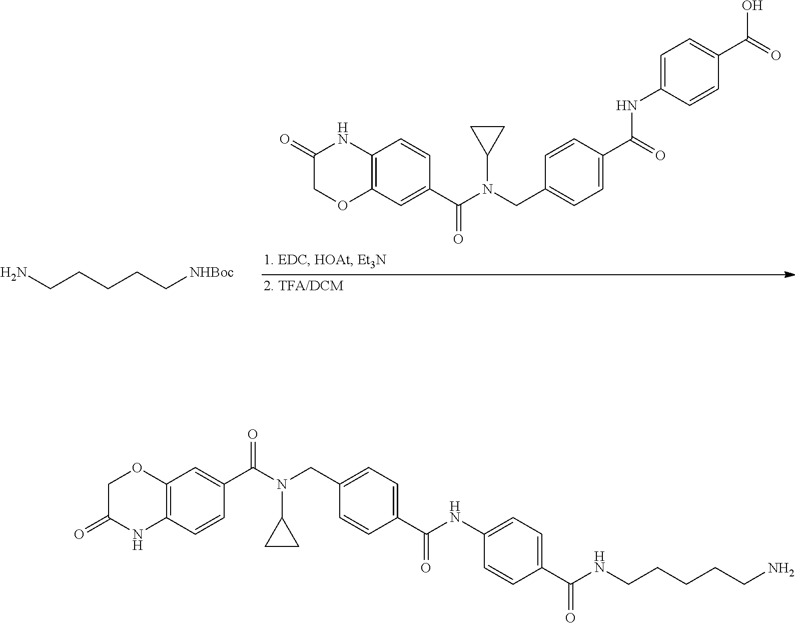 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCCCCN ; NCCCCCNC(=O)c1ccc(NC(=O)c2ccc(CN(C(=O)c3ccc4c(c3)OCC(=O)N4)C3CC3)cc2)cc1]

To a 2-dram vial were added 4-(4-((N-cyclopropyl-3-oxo-3,4-dihydro-2H-benzo[b][1,4]oxazine-7-carboxamido)methyl)benzamido)benzoic acid (20 mg, 1 Eq, 41 μmol), HOAt (8.4 mg, 1.5 Eq, 62 μmol), EDC (12 mg, 1.5 Eq, 62 μmol), and DMF (0.5 mL). The reaction was left to stir at room temperature for 30 minutes. To the vial was then added Boc-DAPe-OH (10 mg, 1.2 Eq, 49 μmol) dissolved in DMF (0.5 mL), followed by triethylamine (8.8 mg, 12 μL, 2.1 Eq, 87 μmol), and the reaction was left to stir overnight.

The next day, the reaction was diluted with water and the white precipitate formed was isolated by centrifugation, washed once with water, then taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((5-aminopentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (26.6 mg, 38.9 μmol, 94%) as a white solid. ¹H NMR (400 MHz, Methanol-d₄) δ 7.95 (d, J=8.4 Hz, 2H), 7.84 (s, 4H), 7.50 (d, J=7.9 Hz, 2H), 7.20 (dd, J=8.0, 1.7 Hz, 1H), 7.17 (d, J=1.7 Hz, 1H), 6.96 (d, J=8.0 Hz, 1H), 4.83 (s, 2H), 4.62 (s, 2H), 3.41 (t, J=7.0 Hz, 2H), 2.94 (t, J=7.6 Hz, 2H), 2.88-2.78 (m, 1H), 1.70 (tt, J=13.0, 7.4 Hz, 4H), 1.49 (q, J=8.3 Hz, 2H), 0.73-0.61 (m, 2H), 0.61-0.48 (m, 2H). LCMS (ESI, +ve mode) expected m/z for [M+H] 570.27, found 570.25.

### Example 137

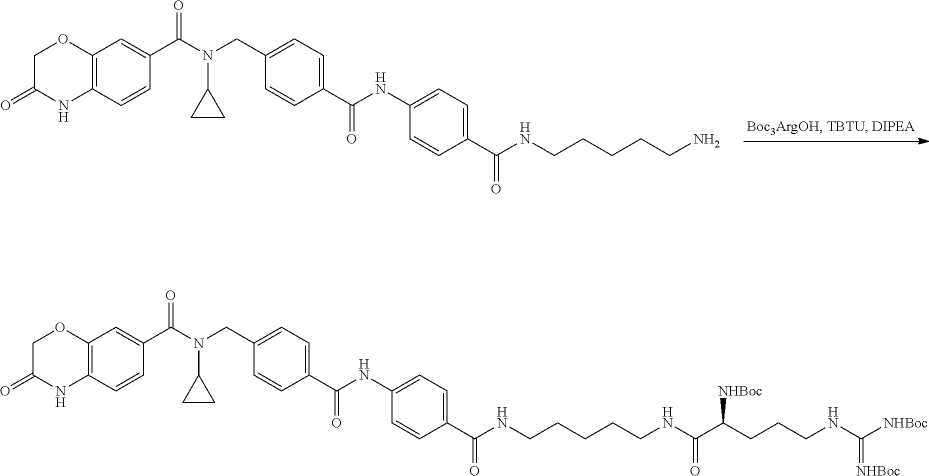

To a 2-dram vial were added (S,E)-5-(2,3-Bis(tert-butoxycarbonyl)guanidino)-2-((tert-butoxycarbonyl)amino)pentanoic acid (3.8 mg, 1.1 Eq, 8.0 μmol), 2-(1H-benzo[d][1,2,3]triazol-1-yl)-1,1,3,3-tetramethylisouronium tetrafluoroborate (3.1 mg, 1.3 Eq, 9.5 μmol), N-ethyl-N-isopropylpropan-2-amine (3.1 mg, 3.3 Eq, 24 μmol), and DMF (0.2 mL). The reaction was left to stir at room temperature for 30 minutes. To the vial was then added N-(4-((4-((5-aminopentyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5.0 mg, 1 Eq, 7.3 μmol) dissolved in DMF (0.2 mL), and the reaction was left to stir overnight.

The next day, the reaction was quenched with water and extracted 3 times with ethyl acetate.

The combined organic fractions were washed once with twice with water, once with saturated sodium bicarbonate, and once with brine, then dried by passage through a phase separator and concentrated to a residue. Normal phase chromatography over silica gel (0-10% MeOH in DCM) provided Product V (7.2 mg, 7.0 μmol, 96%) as a white solid. ¹H NMR (400 MHz, Chloroform-d) δ 8.97 (s, 1H), 8.85 (s, 1H), 7.89 (d, J=8.2 Hz, 2H), 7.83-7.75 (m, 4H), 7.36 (d, J=7.9 Hz, 2H), 7.13 (s, 1H), 7.11 (d, J=8.1 Hz, 1H), 6.99-6.92 (m, 1H), 6.84 (d, J=8.0 Hz, 1H), 6.74 (t, J=5.6 Hz, 1H), 5.89 (d, J=8.9 Hz, 1H), 4.76 (s, 2H), 4.61 (s, 2H), 4.21-4.14 (m, 1H), 3.93-3.82 (m, 1H), 3.71-3.60 (m, 1H), 3.49-3.34 (m, 2H), 3.32-3.18 (m, 2H), 2.64 (tt, J=7.0, 3.9 Hz, 1H), 1.66-1.51 (m, 7H), 1.49 (s, 9H), 1.45 (s, 9H), 1.44 (s, 9H), 1.35 (q, J=7.3 Hz, 3H), 0.66-0.56 (m, 2H), 0.55-0.46 (m, 2H). LCMS (ESI, +ve mode) expected m/z for [M+H]⁺ 1026.53, found 1026.40.

### Example 138

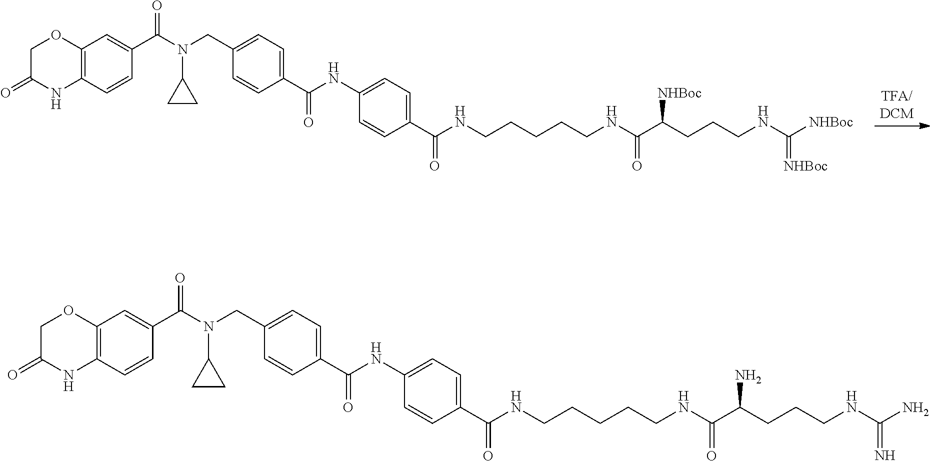

To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((4-((5-(2-amino-5-guanidinopentanamido)pentyl)carbamoyl) phenyl) carbamoyl)benzyl)-N-cyclopropyl-3-oxo-3,4-dihydro-2H-benzo[b][1,4]oxazine-7-carboxamide bis(2,2,2-trifluoroacetate) (3.23 mg, 3.39 μmol, 97.3%) as a white solid. ¹H NMR (400 MHz, Methanol-d₄) δ 7.96 (d, J=8.2 Hz, 2H), 7.87-7.82 (m, 4H), 7.51 (d, J=7.9 Hz, 2H), 7.19 (d, J=8.4 Hz, 1H), 7.18 (s, 1H), 6.97 (d, J=8.0 Hz, 1H), 4.84 (s, 2H), 4.63 (s, 2H), 3.82 (t, J=6.5 Hz, 1H), 3.40 (td, J=7.0, 2.9 Hz, 2H), 3.37-3.33 (m, 1H), 3.28-3.24 (m, 1H), 3.24-3.19 (m, 2H), 2.88-2.79 (m, 1H), 1.94-1.82 (m, 2H), 1.71-1.58 (m, 6H), 1.49-1.39 (m, 2H), 0.71-0.61 (m, 2H), 0.61-0.51 (m, 2H). LCMS (ESI, +ve mode) expected m/z for [M+H]⁺ 726.37, found 726.30; expected m/z for [M+2H]²⁺ 363.69, found 363.80.

### Example 139

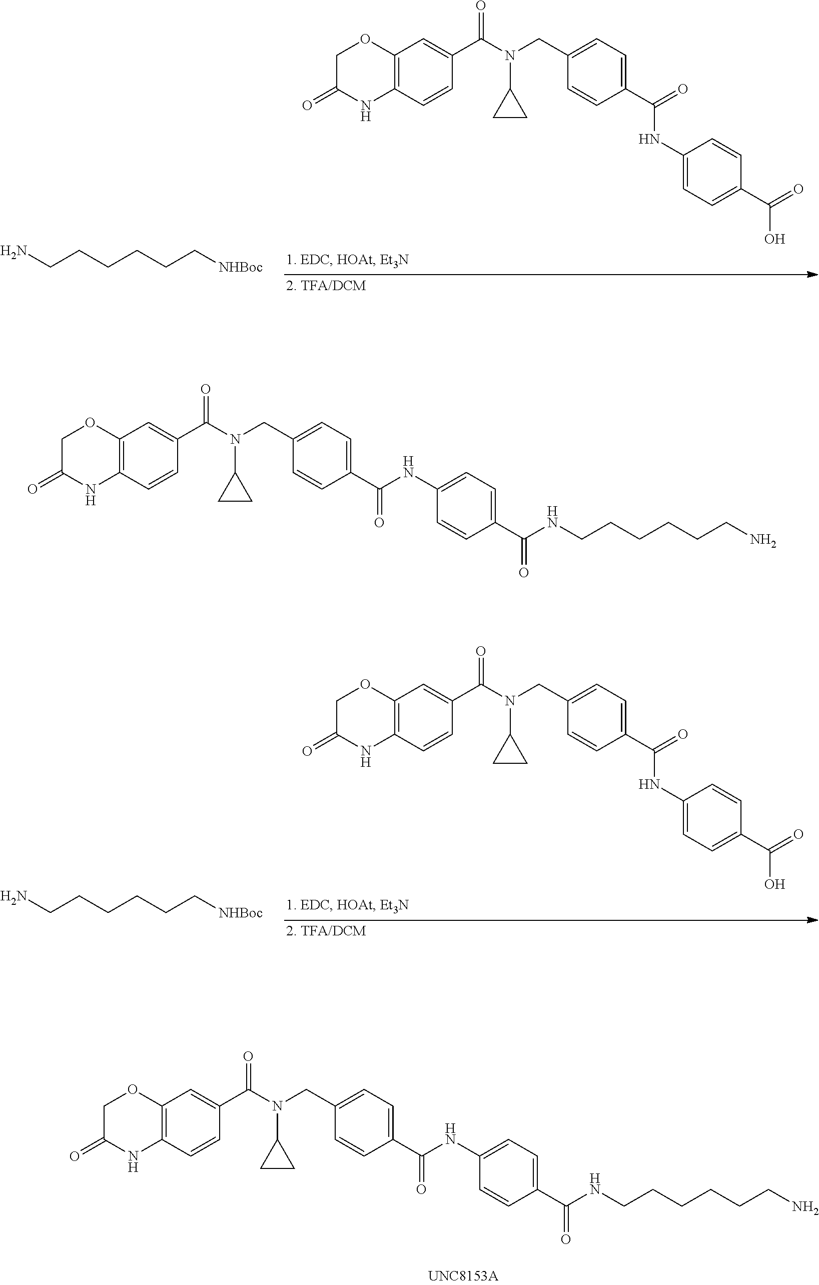 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCCCCCN ; NCCCCCCNC(=O)c1ccc(NC(=O)c2ccc(CN(C(=O)c3ccc4c(c3)OCC(=O)N4)C3CC3)cc2)cc1 ; O=C1COc2cc(C(=O)N(Cc3ccc(C(=O)Nc4ccc(C(=O)O)cc4)cc3)C3CC3)ccc2N1 ; CC(C)(C)OC(=O)NCCCCCCN ; NCCCCCCNC(=O)c1ccc(NC(=O)c2ccc(CN(C(=O)c3ccc4c(c3)OCC(=O)N4)C3CC3)cc2)cc1]

To a 2-dram vial were added 4-(4-((N-cyclopropyl-3-oxo-3,4-dihydro-2H-benzo[b][1,4]oxazine-7-carboxamido)methyl)benzamido)benzoic acid (200 mg, 1 Eq, 412 mol), 3-(((ethylimino)methylene)amino)-N,N-dimethylpropan-1-amine hydrochloride (118 mg, 1.5 Eq, 618 μmol), HOAt (84.1 mg, 1.5 Eq, 618 μmol), and DMF (1 mL). The reaction was left to stir at room temperature for 30 minutes. To the vial was then added tert-butyl (6-aminohexyl)carbamate (134 mg 1.5 Eq, 618 μmol) dissolved in DMF (1 mL), followed by triethylamine (167 mg, 0.23 mL, 4 Eq, 1.65 mmol), and the reaction was left to stir overnight.

The next day, the reaction was diluted with water and the white precipitate formed was isolated by centrifugation, washed once with water, taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (139.8 mg, 200.4 μmol, 48.6%) as a white solid. ¹H NMR (400 MHz, Methanol-d₄) δ 7.94 (d, J=8.3 Hz, 2H), 7.89-7.78 (m, 4H), 7.47 (d, J=7.9 Hz, 2H), 7.18 (dd, J=8.2, 1.7 Hz, 1H), 7.15 (d, J=1.7 Hz, 1H), 6.95 (d, J=8.1 Hz, 1H), 4.80 (s, 2H), 4.60 (s, 2H), 3.39 (t, J=7.1 Hz, 2H), 2.92 (t, J=7.6 Hz, 2H), 2.86-2.74 (m, 1H), 1.73-1.59 (m, 4H), 1.49-1.38 (m, 4H), 0.70-0.58 (m, 2H), 0.58-0.47 (m, 2H). ¹³C NMR (101 MHz, cd₃od) δ 169.53, 168.54, 167.31, 144.59, 143.58, 143.12, 135.05, 131.10, 129.89, 129.16, 129.06, 128.85, 123.21, 121.28, 116.89, 116.71, 68.11, 52.25, 40.67, 40.61, 30.29, 28.44, 27.39, 27.00, 10.46. LCMS (ESI, +ve mode) expected m/z for [M+H]⁺ 584.29, found 584.20.

### Example 140

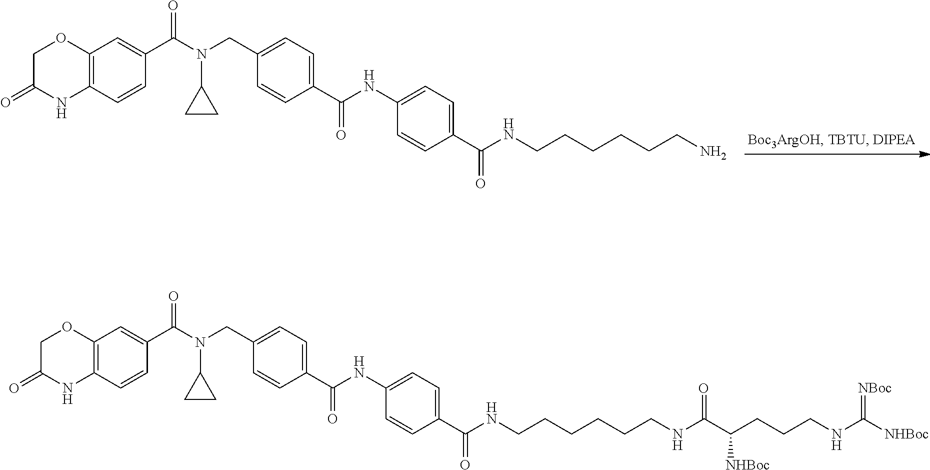

To a 2-dram vial were added (S)-2-((tert-butoxycarbonyl)amino)-5-((2,2,10,10-tetramethyl-4,8-dioxo-3,9-dioxa-5,7-diazaundecan-6-ylidene)amino)pentanoic acid (4 mg, 1.1 Eq, 8 μmol), TBTU (3 mg, 1.3 Eq, 9 μmol), DIPEA (2 mg, 3 μL, 2.3 Eq, 0.02 mmol), and DMF (0.2 mL). The reaction was left to stir at room temperature for 15 minutes. To the vial was then added N-(4-((4-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5 mg, 1 Eq, 7 μmol) dissolved in DMF (0.2 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (8.9 mg, 8.6 μmol, 100%) as a clear gum. ¹H NMR (400 MHz, Chloroform-d) δ 8.89 (s, 1H), 8.83 (s, 1H), 7.90 (d, J=8.2 Hz, 2H), 7.77 (s, 4H), 7.36 (d, J=7.9 Hz, 2H), 7.15-7.07 (m, 2H), 6.96-6.90 (m, 1H), 6.84 (d, J=8.0 Hz, 1H), 6.69 (t, J=5.8 Hz, 1H), 5.91 (d, J=8.7 Hz, 1H), 4.76 (s, 2H), 4.60 (s, 2H), 4.22-4.15 (m, 1H), 4.00-3.87 (m, 1H), 3.73-3.63 (m, 1H), 3.47-3.33 (m, 2H), 3.33-3.14 (m, 2H), 2.68-2.61 (m, 1H), 1.68 (s, 4H), 1.63-1.53 (m, 4H), 1.50 (s, 9H), 1.47 (s, 9H), 1.44 (s, 9H), 1.38-1.29 (m, 4H), 0.66-0.58 (m, 2H), 0.54-0.46 (m, 2H). LCMS (ESI, +ve mode) expected m/z for [M+H]⁺ 1040.55, found 1040.40.

### Example 141

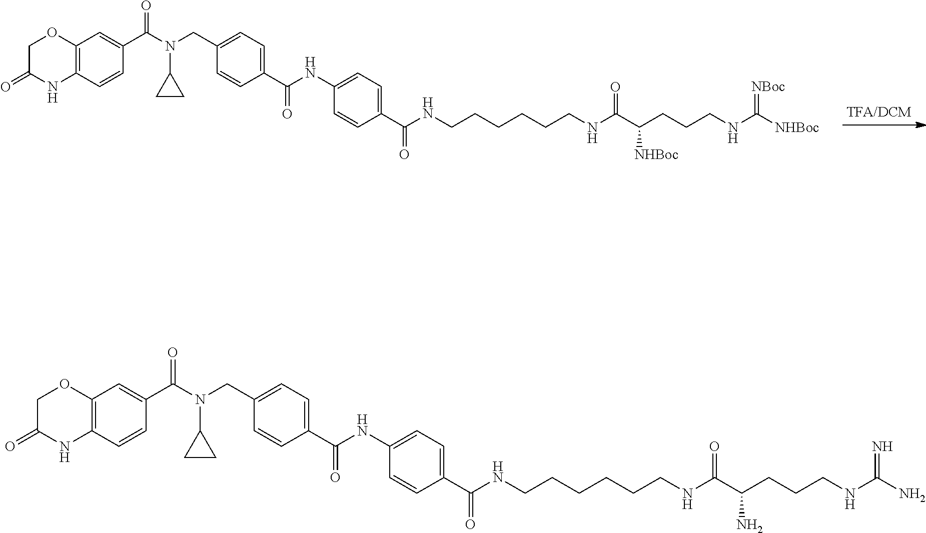

To a vial containing Reactant I (3.8 mg, 1 Eq, 3.7 μmol) was added DCM (500 μL) and TFA (740 mg, 500 μL, 1.8e+3 Eq, 6.49 mmol), and the reaction was stirred at room temperature overnight. The next day, the volatiles were removed under reduced pressure and the residue purified by reverse phase chromatography (10-100% methanol in water+0.1% TFA) followed by lyophilization to yield (S)—N-(4-((4-((6-(2-amino-5-((diaminomethylene)amino) pentanamido)hexyl)carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate) (4.05 mg, 4.18 μmol, 1.1e+2%) as a white solid. ¹H NMR (400 MHz, Methanol-d₄) δ 7.96 (d, J=8.1 Hz, 2H), 7.84 (s, 3H), 7.51 (d, J=7.9 Hz, 2H), 7.20 (d, J=8.6 Hz, 1H), 7.18 (s, 1H), 6.97 (d, J=8.0 Hz, 1H), 4.84 (s, 2H), 4.63 (s, 2H), 3.83 (t, J=6.6 Hz, 1H), 3.39 (t, J=7.0 Hz, 2H), 3.29-3.26 (m, 1H), 3.24 (t, J=7.0 Hz, 2H), 2.89-2.79 (m, 1H), 1.96-1.82 (m, 2H), 1.72-1.52 (m, 6H), 1.49-1.37 (m, 4H), 0.71-0.61 (m, 2H), 0.61-0.51 (m, 2H). LCMS (ESI, +ve mode) expected m/z for [M+H]⁺ 740.39, found 740.30; expected m/z for [M+2H]²+370.70, found 370.80.

### Example 142

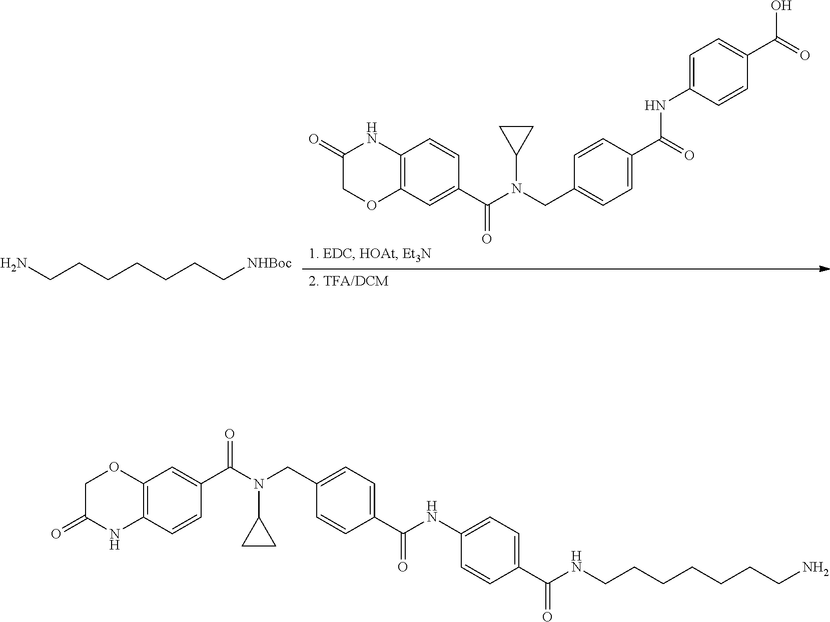 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCCCCCCN ; NCCCCCCCNC(=O)c1ccc(NC(=O)c2ccc(CN(C(=O)c3ccc4c(c3)OCC(=O)N4)C3CC3)cc2)cc1]

To a 2-dram vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt(25 mg, 1.8 Eq, 0.19 mmol), and DMF (0.2 mL). The reaction was left to stir at room temperature for 30 minutes. To the vial was then added tert-butyl (7-aminoheptyl)carbamate (36 mg, 1.5 Eq, 0.15 mmol) dissolved in DMF (0.5 mL), followed by triethylamine (31 mg, 43 μL, 3 Eq, 0.31 mmol), and the reaction was left to stir overnight.

The next day, the reaction was diluted with water and the white precipitate formed was isolated by centrifugation, washed once with water, taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (15.3 mg, 21.5 μmol, 21%) as a white solid. ¹H NMR (400 MHz, Methanol-d₄) δ 7.96 (d, J=8.3 Hz, 2H), 7.87-7.80 (m, 4H), 7.56-7.44 (m, 2H), 7.20 (dd, J=8.1, 1.8 Hz, 1H), 7.17 (d, J=1.7 Hz, 1H), 6.96 (d, J=8.1 Hz, 1H), 4.83 (s, 2H), 4.62 (s, 2H), 3.39 (t, J=7.1 Hz, 2H), 2.92 (t, J=7.6 Hz, 2H), 2.88-2.78 (m, 1H), 1.71-1.59 (m, 4H), 1.48-1.37 (m, 6H), 0.71-0.61 (m, 2H), 0.61-0.49 (m, 2H). ¹³C NMR (101 MHz, Methanol-d₄) δ 169.56, 168.62, 167.36, 144.67, 143.15, 135.13, 131.20, 129.97, 129.16, 129.05, 128.91, 123.23, 121.30, 116.92, 116.71, 68.16, 40.82, 40.71, 30.42, 29.80, 28.51, 27.74, 27.34, 10.56. LCMS (ESI, +ve mode) expected m/z for [M+H]⁺ 598.30, found 598.30.

### Example 143

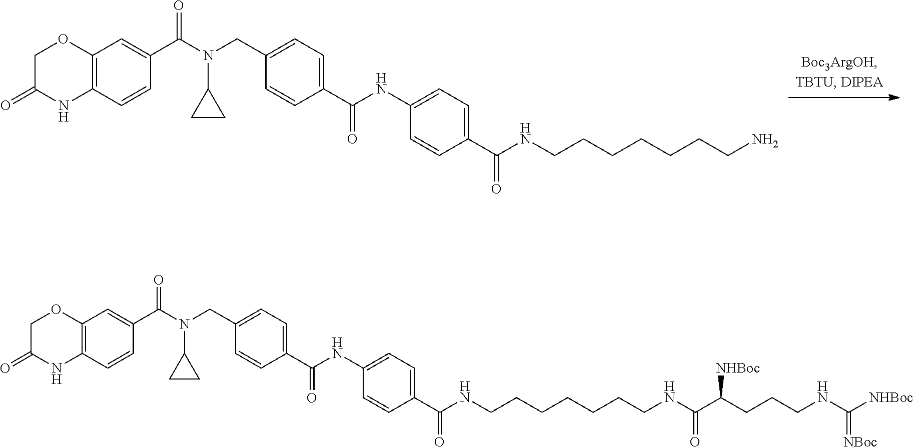

To a 2-dram vial were added (Z)—N2,Nw,Nw′-tris(tert-butoxycarbonyl)-L-arginine (8.0 mg 1.2 Eq, 17 μmol), TBTU (5.9 mg, 1.3 Eq, 18 μmol), DIPEA (6.0 mg, 8.1 μL, 3.3 Eq, 46 μmol), and DMF (0.5 mL). The reaction was left to stir at room temperature for 15 minutes. To the vial was then added N-(4-((4-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol) dissolved in DMF (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (14.04 mg, 13.32 μmol, 95%) as a clear gum. ¹H NMR (400 MHz, Chloroform-d) δ 9.29 (s, 1H), 9.06 (s, 1H), 7.88 (d, J=8.0 Hz, 2H), 7.77-7.70 (m, 4H), 7.32 (d, J=7.9 Hz, 2H), 7.12-7.10 (m, 1H), 7.10-7.06 (m, 1H), 7.01-6.94 (m, 1H), 6.85 (d, J=8.0 Hz, 1H), 6.76 (t, J=5.7 Hz, 1H), 5.98 (d, J=8.7 Hz, 1H), 4.74 (s, 2H), 4.59 (s, 2H), 4.24-4.16 (m, 1H), 3.97-3.88 (m, 1H), 3.73-3.62 (m, 1H), 3.37 (q, J=6.6, 6.1 Hz, 2H), 3.32-3.22 (m, 1H), 3.20-3.10 (m, 1H), 2.65 (tt, J=7.1, 4.1 Hz, 1H), 1.94-1.75 (m, 4H), 1.75-1.62 (m, 4H), 1.60-1.52 (m, 3H), 1.49 (s, 9H), 1.47 (s, 9H), 1.42 (s, 9H), 1.35-1.19 (m, 9H), 0.66-0.57 (m, 2H), 0.54-0.44 (m, 2H). LCMS (ESI, +ve mode) expected m/z for [M+H]⁺ 1054.56, found 1054.40.

### Example 144

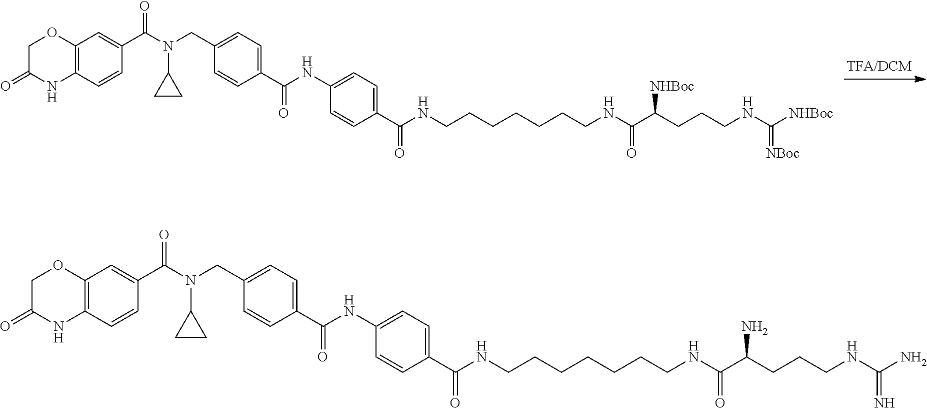

To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((4-((7-(2-amino-5-guanidinopentanamido)heptyl)carbamoyl)phenyl) carbamoyl)benzyl)-N-cyclopropyl-3-oxo-3,4-dihydro-2H-benzo[b][1,4]oxazine-7-carboxamide bis(2,2,2-trifluoroacetate) (5.4 mg, 5.5 μmol, 61%) as a white solid. ¹H NMR (400 MHz, Methanol-d₄) δ 7.96 (d, J=8.2 Hz, 2H), 7.87-7.81 (m, 4H), 7.50 (d, J=7.9 Hz, 2H), 7.20 (d, J=8.6 Hz, 1H), 7.18 (s, 1H), 6.97 (d, J=8.1 Hz, 1H), 4.84 (s, 2H), 4.63 (s, 2H), 3.83 (t, J=6.6 Hz, 1H), 3.38 (t, J=7.1 Hz, 2H), 3.29-3.19 (m, 4H), 2.88-2.79 (m, 1H), 1.93-1.83 (m, 2H), 1.71-1.59 (m, 4H), 1.59-1.51 (m, 2H), 1.47-1.34 (m, 6H), 0.71-0.61 (m, 2H), 0.61-0.51 (m, 2H). LCMS (ESI, +ve mode) expected m/z for [M+H]⁺ 754.40, found 754.30; expected m/z for [M+2H]²⁺ 377.70, found 377.85.

### Example 145

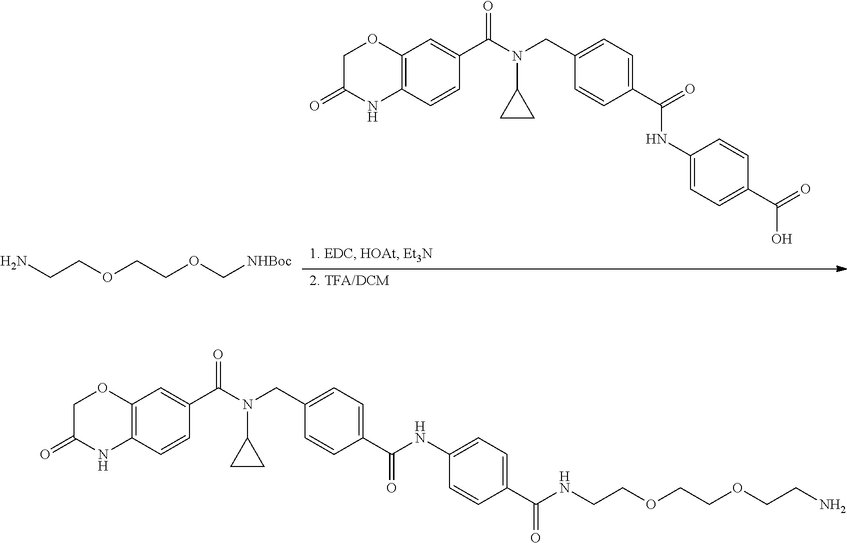 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCOCCOCCN ; NCCOCCOCCNC(=O)c1ccc(NC(=O)c2ccc(CN(C(=O)c3ccc4c(c3)OCC(=O)N4)C3CC3)cc2)cc1]

To a scintillation vial were added 4-(4-((N-cyclopropyl-3-oxo-3,4-dihydro-2H-benzo[b][1,4]oxazine-7-carboxamido)methyl)benzamido)benzoic acid (20.0 mg, 1 Eq, 41.2 μmol), 3-(((ethylimino)methylene)amino)-N,N-dimethylpropan-1-amine hydrochloride (15.8 mg, 2 Eq, 82.4 μmol), HOAt (11.2 mg, 2 Eq, 82.4 μmol), and DMF (0.2 mL). The reaction was left to stir at room temperature for 30 minutes. To the vial was then added tert-butyl (2-(2-(2-aminoethoxy)ethoxy)ethyl)carbamate (13.3 mg, 1.3 Eq, 53.6 μmol) dissolved in DMF (0.2 mL), followed by triethylamine (16.7 mg, 23 μL, 4 Eq, 165 μmol), and the reaction was left to stir overnight. The next day, the residue was partitioned between water and ethyl acetate. The layers were separated, and the aqueous layer was extracted twice more with ethyl acetate. The combined organic layers were washed once with 0.5M citric acid, once with water, once with saturated sodium bicarbonate, and once with brine, then dried over sodium sulfate and concentrated to an off-white residue.

To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (18.7 mg, 25.6 μmol, 62.2%) as a white solid. ¹H NMR (400 MHz, Methanol-d₄) δ 7.96 (d, J=8.3 Hz, 2H), 7.89-7.81 (m, 4H), 7.55-7.44 (m, 2H), 7.20 (dd, J=8.0, 1.7 Hz, 1H), 7.17 (d, J=1.8 Hz, 1H), 6.96 (d, J=8.0 Hz, 1H), 4.83 (s, 2H), 4.62 (s, 2H), 3.69 (tt, J=5.1, 2.1 Hz, 8H), 3.60 (t, J=5.6 Hz, 2H), 3.08 (t, J=5.0 Hz, 2H), 2.87-2.78 (m, 1H), 0.71-0.61 (m, 2H), 0.61-0.48 (m, 2H).

### Example 146

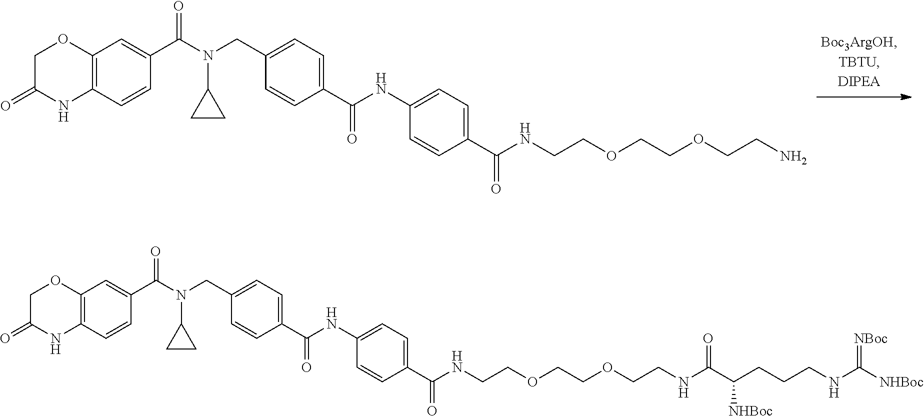

To a 2-dram vial were added (S)-2-((tert-butoxycarbonyl)amino)-5-((2,2,10,10-tetramethyl-4,8-dioxo-3,9-dioxa-5,7-diazaundecan-6-ylidene)amino)pentanoic acid (3.6 mg, 1.1 Eq, 7.5 μmol), TBTU (2.9 mg, 1.3 Eq, 8.9 μmol), DIPEA (2.0 mg, 2.8 μL, 2.3 Eq, 16 μmol), and DMF (0.2 mL). The reaction was left to stir at room temperature for 15 minutes. To the vial was then added N-(4-((4-((2-(2-(2-aminoethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5.0 mg, 1 Eq, 6.9 μmol) dissolved in DMF (0.2 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (6.9 mg, 6.4 μmol, 94%) as a clear gum. ¹H NMR (400 MHz, Chloroform-d) δ 9.15 (s, 1H), 8.40 (s, 1H), 8.00 (d, J=7.9 Hz, 2H), 7.87 (d, J=8.7 Hz, 2H), 7.78 (d, J=8.3 Hz, 2H), 7.41 (d, J=7.9 Hz, 2H), 7.31 (t, J=5.5 Hz, 1H), 7.17-7.11 (m, 2H), 6.82 (d, J=8.0 Hz, 2H), 5.40 (d, J=8.6 Hz, 1H), 4.78 (s, 2H), 4.62 (s, 2H), 3.92-3.74 (m, 2H), 3.71-3.48 (m, 10H), 3.46-3.29 (m, 2H), 2.67-2.58 (m, 1H), 1.64-1.52 (m, 2H), 1.44 (s, 9H), 1.42 (s, 9H), 1.29-1.22 (m, 2H), 0.64-0.57 (m, 2H), 0.53-0.46 (m, 2H). LCMS (ESI, +ve mode) expected m/z for [M+H]⁺ 1072.53, found 1072.40.

### Example 147

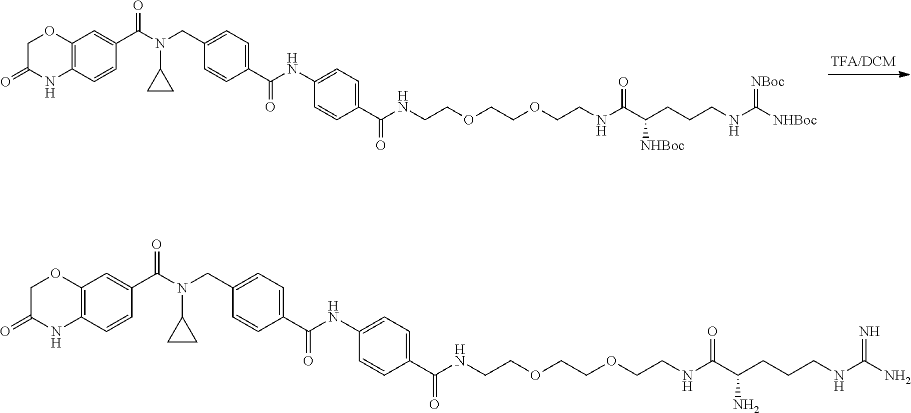

To a vial containing Reactant I (3.0 mg, 1 Eq, 2.8 μmol) was added DCM (500 μL) and TFA (740 mg, 500 μL, 2.3e+3 Eq, 6.49 mmol) and stirred at room temperature overnight. The next day, the volatiles were removed under reduced pressure and the residue was purified by reverse phase chromatography (10-100% methanol in water+0.1%) to yield (S)—N-cyclopropyl-3-oxo-N-(4-((4-((1,1,6-triamino-7-oxo-11,14-dioxa-2,8-diazahexadec-1-en-16-yl)carbamoyl)phenyl)carbamoyl)benzyl)-3,4-dihydro-2H-benzo[b][1,4]oxazine-7-carboxamide bis(2,2,2-trifluoroacetate) (3.41 mg, 3.41 μmol, 1.2e+2%). ¹H NMR (400 MHz, Methanol-d₄) δ 7.96 (d, J=8.2 Hz, 2H), 7.89-7.81 (m, 4H), 7.51 (d, J=7.9 Hz, 2H), 7.21 (d, J=8.4 Hz, 1H), 7.19-7.16 (m, 1H), 6.97 (d, J=8.0 Hz, 1H), 4.84 (s, 2H), 4.63 (s, 2H), 3.86 (t, J=6.5 Hz, 1H), 3.72-3.55 (m, 9H), 3.51-3.34 (m, 3H), 3.22 (t, J=7.0 Hz, 2H), 2.89-2.79 (m, 1H), 1.88 (dt, J=13.8, 6.8 Hz, 2H), 1.67 (p, J=7.5 Hz, 2H), 0.71-0.61 (m, 2H), 0.61-0.51 (m, 2H). LCMS (ESI, +ve mode) expected m/z for [M+H]⁺ 772.38, found 772.30; expected m/z for [M+2H]²⁺ 386.69, found 386.80.

### Example 148

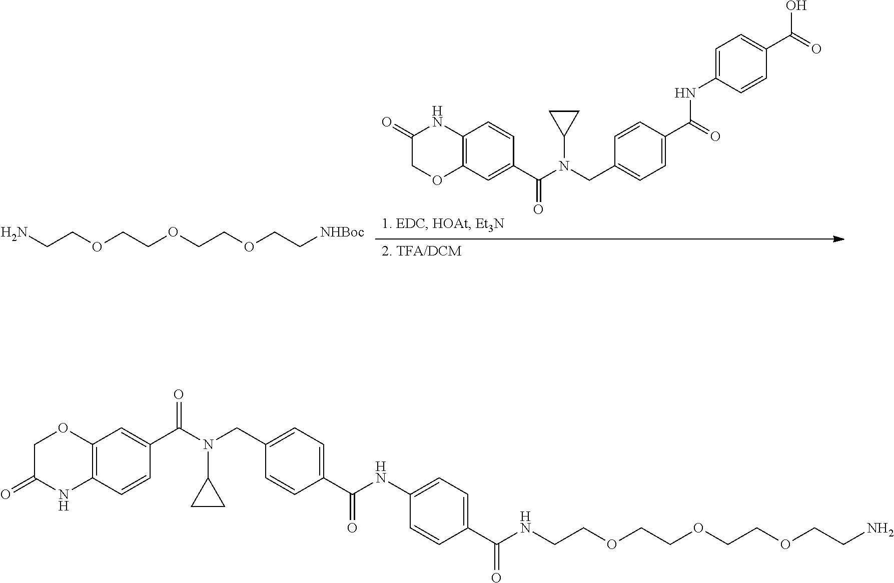 [SMILES: O=C1COc2cc(C(=O)N(Cc3ccc(C(=O)Nc4ccc(C(=O)O)cc4)cc3)C3CC3)ccc2N1 ; CC(C)(C)OC(=O)NCCOCCOCCOCCN ; NCCOCCOCCOCCNC(=O)c1ccc(NC(=O)c2ccc(CN(C(=O)c3ccc4c(c3)OCC(=O)N4)C3CC3)cc2)cc1]

To a scintillation vial were added 4-(4-((N-cyclopropyl-3-oxo-3,4-dihydro-2H-benzo[b][1,4]oxazine-7-carboxamido)methyl)benzamido)benzoic acid (20.0 mg, 1 Eq, 41.2 μmol), 3-(((ethylimino)methylene)amino)-N,N-dimethylpropan-1-amine hydrochloride (15.8 mg, 2 Eq, 82.4 μmol), HOAt (11.2 mg, 2 Eq, 82.4 μmol), and DMF (0.2 mL). The reaction was left to stir at room temperature for 30 minutes. To the vial was then added tert-butyl (2-(2-(2-(2-aminoethoxy)ethoxy)ethoxy)ethyl)carbamate (15.7 mg, 1.3 Eq, 53.6 μmol) dissolved in DMF (0.2 mL), followed by triethylamine (16.7 mg, 23 μL, 4 Eq, 165 μmol), and the reaction was left to stir overnight. The next day, the residue was partitioned between water and ethyl acetate. The layers were separated, and the aqueous layer was extracted twice more with ethyl acetate. The combined organic layers were washed once with 0.5M citric acid, once with water, once with saturated sodium bicarbonate, and once with brine, then dried over sodium sulfate and concentrated to an off-white residue.

To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((2-(2-(2-(2-aminoethoxy)ethoxy)ethoxy)ethyl)carbamoyl)phenyl) carbamoyl)benzyl)-N-cyclopropyl-3-oxo-3,4-dihydro-2H-benzo[b][1,4]oxazine-7-carboxamide 2,2,2-trifluoroacetate (17 mg, 22 μmol, 53%) as a white solid. ¹H NMR (400 MHz, Methanol-d₄) δ 7.96 (d, J=8.3 Hz, 2H), 7.89-7.81 (m, 4H), 7.55-7.45 (m, 2H), 7.20 (dd, J=8.0, 1.8 Hz, 1H), 7.17 (d, J=1.7 Hz, 1H), 6.96 (d, J=8.1 Hz, 1H), 4.83 (s, 2H), 4.62 (s, 2H), 3.71-3.63 (m, 12H), 3.59 (t, J=5.5 Hz, 2H), 3.11 (t, J=5.0 Hz, 2H), 2.88-2.78 (m, 1H), 0.72-0.61 (m, 2H), 0.61-0.48 (m, 2H).

### Example 149

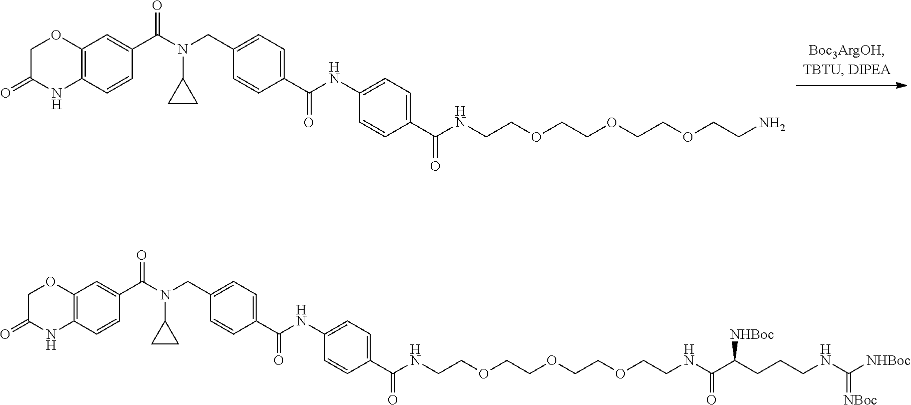

To a 2-dram vial were added (S)-2-((tert-butoxycarbonyl)amino)-5-((2,2,10,10-tetramethyl-4,8-dioxo-3,9-dioxa-5,7-diazaundecan-6-ylidene)amino)pentanoic acid (3.4 mg, 1.1 Eq, 7.1 μmol), TBTU (2.7 mg, 1.3 Eq, 8.4 μmol), DIPEA (1.9 mg, 2.6 μL, 2.3 Eq, 15 μmol), and DMF (0.2 mL). The reaction was left to stir at room temperature for 15 minutes. To the vial was then added N-(4-((4-((2-(2-(2-(2-aminoethoxy)ethoxy)ethoxy)ethyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (5.0 mg, 1 Eq, 6.5 μmol) dissolved in DMF (0.2 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (9.6 mg, 8.6 μmol, 130%) as a clear gum. ¹H NMR (400 MHz, Chloroform-d) δ 8.51 (s, 1H), 7.97 (d, J=7.9 Hz, 2H), 7.86 (s, 4H), 7.41 (d, J=7.8 Hz, 2H), 7.16 (s, 2H), 7.14 (d, J=8.3 Hz, 2H), 6.83 (d, J=8.0 Hz, 1H), 4.77 (s, 2H), 4.62 (s, 2H), 3.79-3.46 (m, 15H), 3.39 (s, 2H), 2.63 (tt, J=7.1, 3.9 Hz, 1H), 1.54-1.38 (m, 30H), 0.62 (d, J=6.6 Hz, 2H), 0.51 (s, 2H). LCMS (ESI, +ve mode) expected m/z for [M+H]⁺ 1116.56, found 1116.45.

### Example 150

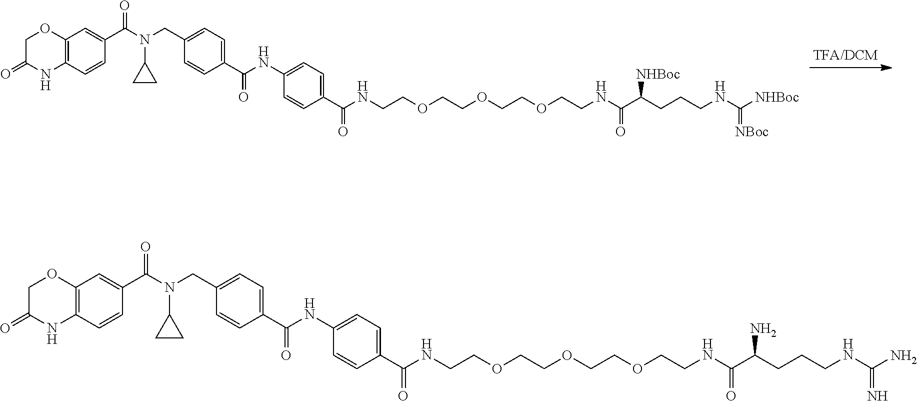

To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-cyclopropyl-3-oxo-N-(4-((4-((1,1,6-triamino-7-oxo-11,14,17-trioxa-2,8-diazanonadec-1-en-19-yl)carbamoyl)phenyl)carbamoyl)benzyl)-3,4-dihydro-2H-benzo[b][1,4]oxazine-7-carboxamide bis(2,2,2-trifluoroacetate) (3.73 mg, 3.57 μmol, 1.1e+2%) as a white solid. ¹H NMR (400 MHz, Methanol-d₄) δ 7.96 (d, J=8.1 Hz, 2H), 7.89-7.81 (m, 4H), 7.51 (d, J=7.9 Hz, 2H), 7.21 (d, J=8.5 Hz, 1H), 7.18 (s, 1H), 6.97 (d, J=8.0 Hz, 1H), 4.84 (s, 2H), 4.63 (s, 2H), 3.86 (t, J=6.6 Hz, 1H), 3.71-3.62 (m, 7H), 3.62-3.53 (m, 5H), 3.50-3.33 (m, 3H), 3.21 (t, J=7.0 Hz, 2H), 2.89-2.80 (m, 1H), 1.93-1.83 (m, 2H), 1.68 (p, J=7.6 Hz, 2H), 0.71-0.62 (m, 2H), 0.61-0.51 (m, 2H). LCMS (ESI, +ve mode) expected m/z for [M+H]⁺ 816.40, found 816.30; expected m/z for [M+2H]²+408.71, found 408.80.

### Example 151

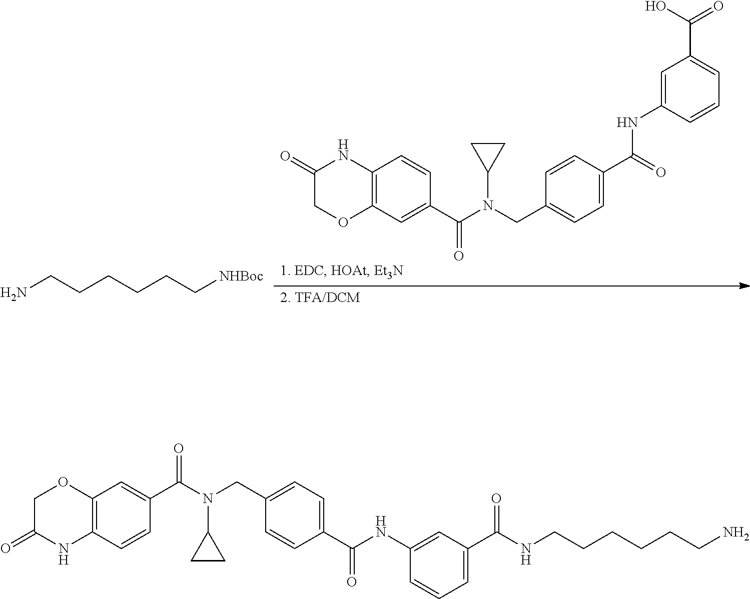 [SMILES: O=C1COc2cc(C(=O)N(Cc3ccc(C(=O)Nc4cccc(C(=O)O)c4)cc3)C3CC3)ccc2N1 ; CC(C)(C)OC(=O)NCCCCCCN ; NCCCCCCNC(=O)c1cccc(NC(=O)c2ccc(CN(C(=O)c3ccc4c(c3)OCC(=O)N4)C3CC3)cc2)c1]

To a 2-dram vial were added 3-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), and DMF (0.5 mL). The reaction was left to stir at room temperature for 30 minutes. To the vial was then added tert-butyl (6-aminohexyl)carbamate (33 mg, 1.5 Eq, 0.15 mmol) dissolved in DMF (0.5 mL), followed by triethylamine (31 mg, 43 μL, 3 Eq, 0.31 mmol), and the reaction was left to stir overnight.

The next day, the reaction was diluted with water and the white precipitate formed was isolated by centrifugation, washed once with water, taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((3-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (58.5 mg, 83.8 μmol, 81%) as a white solid. ¹H NMR (400 MHz, Methanol-d₄) δ 8.20 (t, J=1.9 Hz, 1H), 7.95 (d, J=8.7 Hz, 2H), 7.81 (ddd, J=8.0, 2.2, 1.1 Hz, 1H), 7.57 (d, J=8.0 Hz, 1H), 7.52-7.41 (m, 3H), 7.19 (dd, J=8.0, 1.7 Hz, 1H), 7.16 (d, J=1.7 Hz, 1H), 6.95 (d, J=8.1 Hz, 1H), 4.81 (s, 2H), 4.61 (s, 2H), 3.40 (t, J=7.1 Hz, 2H), 2.92 (t, J=7.6 Hz, 2H), 2.85-2.77 (m, 1H), 1.73-1.58 (m, 4H), 1.50-1.38 (m, 4H), 0.70-0.59 (m, 2H), 0.59-0.47 (m, 2H). ¹³C NMR (101 MHz, cd₃od) δ 170.05, 168.56, 167.33, 144.61, 143.56, 140.17, 136.65, 135.01, 133.29, 130.05, 129.90, 129.13, 128.86, 125.13, 124.10, 123.22, 121.17, 116.89, 116.72, 68.12, 52.21, 40.73, 40.61, 30.21, 28.44, 27.38, 26.99, 10.52. LCMS (ESI, +ve mode) expected m/z for [FORMULA] [M+H] 584.29, found 584.25.

### Example 152

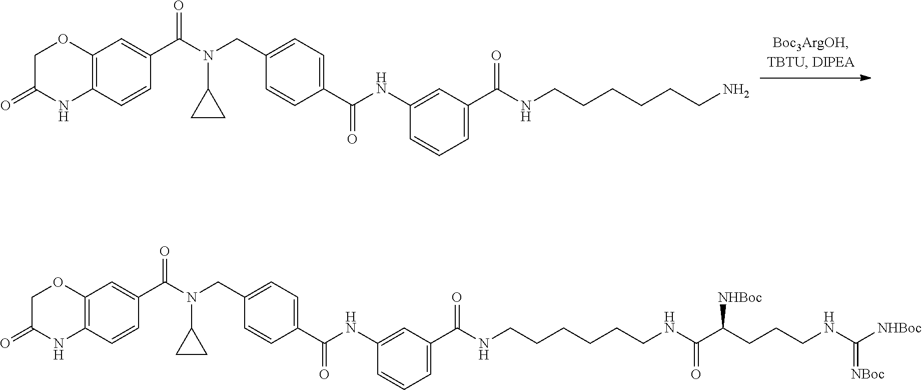

To a 2-dram vial were added Boc3-Arg-OH (11 mg, 1.1 Eq, 24 μmol), 2-(1H-benzo[d][1,2,3]triazol-1-yl)-1,1,3,3-tetramethylisouronium tetrafluoroborate (9.0 mg, 1.3 Eq, 28 μmol), DIPEA (9.2 mg, 12 μL, 3.3 Eq, 71 μmol), and DMF (0.5 mL). The reaction was left to stir at room temperature for 15 minutes. To the vial was then added N-(4-((3-((6-aminohexyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (15 mg, 1 Eq, 21 μmol) dissolved in DMF (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (19.5 mg, 18.7 μmol, 87%) as a clear gum. ¹H NMR (400 MHz, Chloroform-d) δ 9.40 (s, 1H), 9.21 (s, 1H), 8.07 (s, 1H), 8.04 (d, J=8.4 Hz, 1H), 7.91 (d, J=8.1 Hz, 2H), 7.53 (d, J=7.7 Hz, 1H), 7.40-7.31 (m, 3H), 7.13-7.07 (m, 2H), 7.02 (t, J=6.0 Hz, 1H), 6.87 (d, J=7.9 Hz, 1H), 6.85-6.81 (m, 1H), 6.04 (d, J=8.5 Hz, 1H), 4.75 (s, 2H), 4.59 (s, 2H), 4.23-4.14 (m, 1H), 3.94-3.82 (m, 1H), 3.73-3.62 (m, 1H), 3.45-3.25 (m, 3H), 3.24-3.14 (m, 1H), 2.62 (tt, J=6.9, 4.0 Hz, 1H), 1.75-1.51 (m, 8H), 1.48 (s, 9H), 1.47 (s, 9H), 1.41 (s, 9H), 1.37-1.29 (m, 4H), 0.67-0.55 (m, 2H), 0.55-0.43 (m, 2H). LCMS (ESI, +ve mode) expected m/z for [M+H]⁺ 1040.55, found 1040.40.

### Example 153

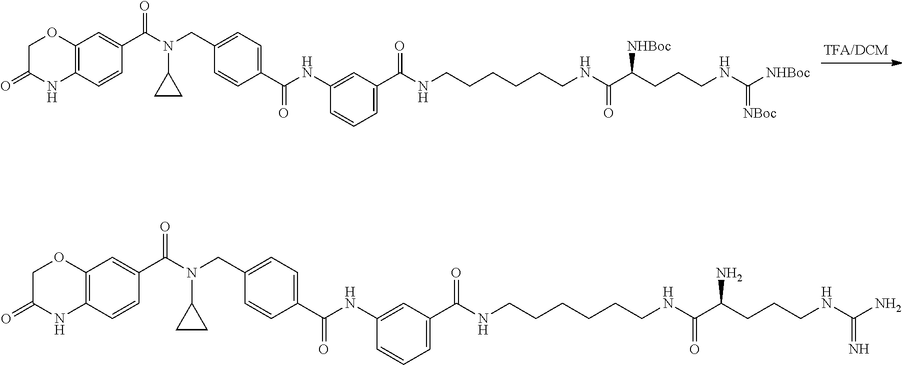

To Reactant I (6.09 mg, 1 Eq, 5.85 μmol) was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((3-((6-(2-amino-5-guanidinopentanamido)hexyl)carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate) (3.81 mg, 3.94 μmol, 67.2%) as a white solid. ¹H NMR (400 MHz, Methanol-d₄) δ 8.24 (t, J=1.9 Hz, 1H), 7.97 (d, J=8.2 Hz, 2H), 7.76 (ddd, J=8.1, 2.2, 1.0 Hz, 1H), 7.58 (dt, J=8.1, 1.3 Hz, 1H), 7.51 (d, J=8.5 Hz, 2H), 7.47 (t, J=7.9 Hz, 1H), 7.21 (d, J=8.3 Hz, 1H), 7.18 (s, 1H), 6.97 (d, J=8.1 Hz, 1H), 4.84 (s, 2H), 4.63 (s, 2H), 3.84 (t, J=6.6 Hz, 1H), 3.40 (td, J=7.0, 3.5 Hz, 2H), 3.30-3.26 (m, 1H), 3.26-3.20 (m, 3H), 2.89-2.80 (m, 1H), 1.95-1.83 (m, 2H), 1.72-1.61 (m, 4H), 1.61-1.53 (m, 2H), 1.49-1.39 (m, 4H), 0.70-0.62 (m, 2H), 0.62-0.52 (m, 2H). LCMS (ESI, +ve mode) expected m/z for [M+H]⁺ 740.39, found 740.30; expected m/z for [M+2H]²⁺ 370.70, found 370.75.

### Example 154

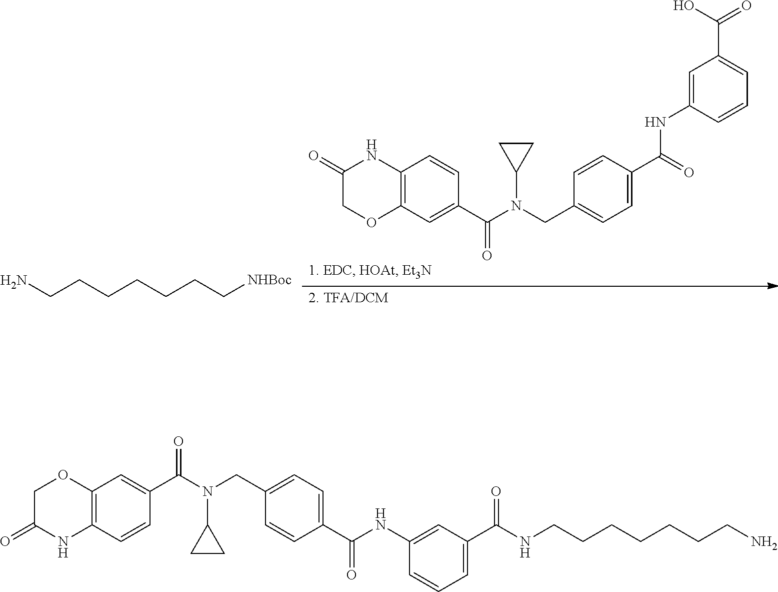 [SMILES: O=C1COc2cc(C(=O)N(Cc3ccc(C(=O)Nc4cccc(C(=O)O)c4)cc3)C3CC3)ccc2N1 ; CC(C)(C)OC(=O)NCCCCCCCN ; NCCCCCCCNC(=O)c1cccc(NC(=O)c2ccc(CN(C(=O)c3ccc4c(c3)OCC(=O)N4)C3CC3)cc2)c1]

To a 2-dram vial were added 3-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (25 mg, 1.8 Eq, 0.19 mmol), and DMF (0.2 mL). The reaction was left to stir at room temperature for 30 minutes. To the vial was then added tert-butyl (7-aminoheptyl)carbamate (36 mg, 1.5 Eq, 0.15 mmol) dissolved in DMF (0.5 mL), followed by triethylamine (31 mg, 43 μL, 3 Eq, 0.31 mmol), and the reaction was left to stir overnight.

The next day, the reaction was diluted with water and the white precipitate formed was isolated by centrifugation, washed once with water, taken up in absolute ethanol and concentrated to a clear residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((3-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (44.6 mg, 62.7 μmol, 61%) as a white solid. ¹H NMR (400 MHz, Methanol-d₄) δ 8.19 (t, J=1.9 Hz, 1H), 7.96 (d, J=8.4 Hz, 2H), 7.81 (ddd, J=8.0, 2.2, 1.1 Hz, 1H), 7.60-7.54 (m, 1H), 7.52-7.41 (m, 3H), 7.19 (dd, J=8.1, 1.8 Hz, 1H), 7.16 (d, J=1.8 Hz, 1H), 6.96 (d, J=8.0 Hz, 1H), 4.82 (s, 2H), 4.61 (s, 2H), 3.39 (t, J=7.1 Hz, 2H), 2.91 (t, J=7.7 Hz, 2H), 2.88-2.78 (m, 1H), 1.71-1.59 (m, 4H), 1.48-1.37 (m, 6H), 0.69-0.60 (m, 2H), 0.60-0.48 (m, 2H). ¹³C NMR (101 MHz, cd₃od) δ 170.04, 168.59, 167.34, 144.63, 143.58, 140.17, 136.72, 135.04, 133.31, 130.05, 129.93, 129.13, 128.88, 125.12, 124.10, 123.23, 121.18, 116.90, 116.72, 68.13, 52.19, 40.88, 40.69, 30.31, 29.76, 28.46, 27.70, 27.31, 10.51. LCMS (ESI, +ve mode) expected m/z for [M+H]⁺ 598.30, found 598.30.

### Example 155

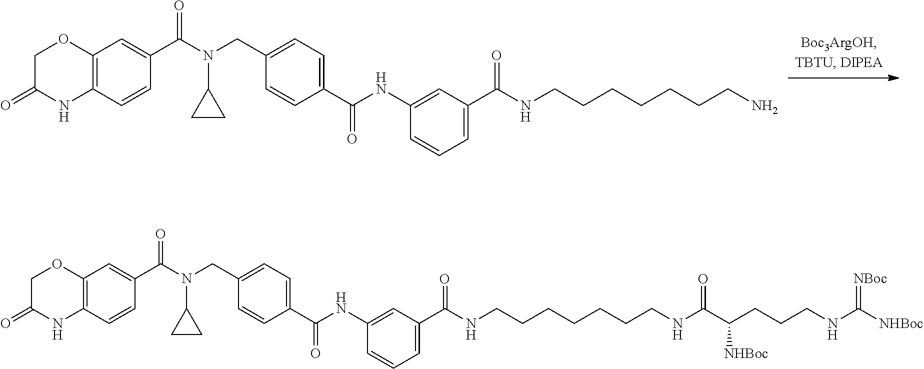

To a 2-dram vial were added (Z)—N2,Nw,Nw′-tris(tert-butoxycarbonyl)-L-arginine (15 mg, 1.1 Eq, 31 μmol), TBTU (12 mg, 1.3 Eq, 37 μmol), DIPEA (13 mg, 17 μL, 3.5 Eq, 98 μmol), and DMF (0.5 mL). The reaction was left to stir at room temperature for 15 minutes. To the vial was then added N-(4-((3-((7-aminoheptyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (20 mg, 1 Eq, 28 μmol) dissolved in DMF (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. Normal phase chromatography over silica gel provided Product V (11.88 mg, 11.27 μmol, 40%) as a clear gum. ¹H NMR (400 MHz, Chloroform-d) δ 9.08 (s, 1H), 8.97 (s, 1H), 8.11 (t, J=1.9 Hz, 1H), 7.99 (d, J=7.9 Hz, 1H), 7.91 (d, J=8.2 Hz, 2H), 7.53 (dt, J=7.8, 1.3 Hz, 1H), 7.43-7.34 (m, 3H), 7.16-7.09 (m, 2H), 7.02-6.95 (m, 1H), 6.88 (d, J=7.9 Hz, 1H), 6.64 (t, J=4.1 Hz, 1H), 5.97 (d, J=8.7 Hz, 1H), 4.76 (s, 2H), 4.61 (s, 2H), 4.23-4.14 (m, 1H), 3.94-3.84 (m, 1H), 3.73-3.63 (m, 1H), 3.45-3.34 (m, 2H), 3.34-3.23 (m, 1H), 3.22-3.11 (m, 1H), 2.63 (tt, J=6.9, 3.9 Hz, 1H), 1.80-1.61 (m, 9H), 1.61-1.53 (m, 4H), 1.49 (s, 9H), 1.47 (s, 9H), 1.41 (s, 9H), 1.37-1.27 (m, 7H), 0.68-0.56 (m, 2H), 0.56-0.45 (m, 2H). LCMS (ESI, +ve mode) expected m/z for [M+H]⁺ 1054.56, found 1054.40.

### Example 156

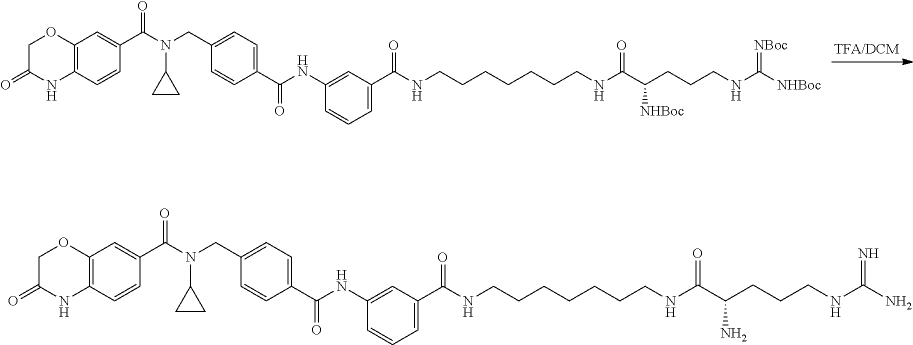

To Reactant I (7.2 mg, 1 Eq, 6.8 mol) was added 5000 TFA in DCM, which was stirred until disappearance of starting material as monitored by LCMS. The reaction was concentrated, co-evaporated with methanol, and purified by reverse phase chromatography (10-100% methanol in water+0.10% TFA) to provide (S)—N-(4-((3-((7-(2-amino-5-guanidinopentanamido)heptyl)carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate)(4.1 mg, 4.2 μmol, 61%) as a white solid. ¹H NMR (400 MHz, Methanol-d₄) δ 8.22 (t, J=1.9 Hz, 1H), 7.97 (d, J=8.2 Hz, 2H), 7.80-7.75 (m, 1H), 7.60-7.55 (m, 1H), 7.51 (d, J=8.2 Hz, 2H), 7.49-7.44 (m, 1H), 7.21 (d, J=8.2 Hz, 1H), 7.18 (s, 1H), 6.97 (d, J=8.1 Hz, 1H), 4.84 (s, 2H), 4.63 (s, 2H), 3.83 (t, J=6.5 Hz, 1H), 3.39 (t, J=7.1 Hz, 2H), 3.29-3.19 (m, 4H), 2.89-2.80 (m, 1H), 1.94-1.82 (m, 2H), 1.71-1.60 (m, 4H), 1.60-1.52 (m, 2H), 1.47-1.34 (m, 6H), 0.70-0.51 (m, 4H). LCMS (ESI, +ve mode) expected m/z for [M+H]⁺ 754.40, found 754.30; m/z for [M+2H]²+377.70, found 377.80.

### Example 157

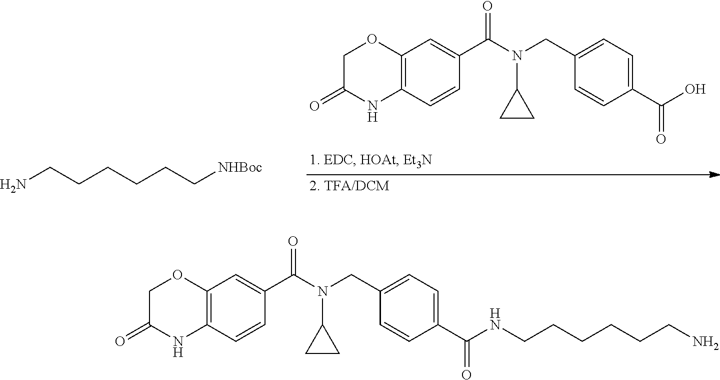 [SMILES: O=C1COc2cc(C(=O)N(Cc3ccc(C(=O)O)cc3)C3CC3)ccc2N1 ; CC(C)(C)OC(=O)NCCCCCCN ; NCCCCCCNC(=O)c1ccc(CN(C(=O)c2ccc3c(c2)OCC(=O)N3)C2CC2)cc1]

To a scintillation vial was added 4-((N-cyclopropyl-3-oxo-3,4-dihydro-2H-benzo[b][1,4]oxazine-7-carboxamido)methyl)benzoic acid (20 mg, 1 Eq, 55 μmol), EDC (16 mg, 1.5 Eq, 82 μmol), HOAt (11 mg, 1.5 Eq, 82 μmol), and DMF (0.5 mL). The reaction was left to stir at room temperature for 30 minutes. To the vial was then added 4-((N-cyclopropyl-3-oxo-3,4-dihydro-2H-benzo[b][1,4]oxazine-7-carboxamido)methyl)benzoic acid (20 mg, 1 Eq, 55 μmol) dissolved in DMF (0.5 mL), followed by triethylamine (12 mg, 16 μL, 2.1 Eq, 0.11 mmol), and the reaction was left to stir overnight. The next day, the reaction was partitioned between water and ethyl acetate. The layers were separated, and the aqueous layer was extracted twice more with ethyl acetate. The combined organic layers were washed once with 0.5M citric acid, once with water, once with saturated sodium bicarbonate, and once with brine, then dried over sodium sulfate and concentrated to an off-white residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((6-aminohexyl)carbamoyl)benzyl)-N-cyclopropyl-3-oxo-3,4-dihydro-2H-benzo[b][1,4]oxazine-7-carboxamide 2,2,2-trifluoroacetate (31.2 mg, 53.9 μmol, 99%) as a white solid. ¹H NMR (400 MHz, Methanol-d₄) δ 7.82 (d, J=8.3 Hz, 2H), 7.47-7.39 (m, 2H), 7.19 (dd, J=7.9, 1.7 Hz, 1H), 7.15 (d, J=1.8 Hz, 1H), 6.96 (d, J=8.1 Hz, 1H), 4.79 (s, 2H), 4.61 (s, 2H), 3.39 (t, J=7.1 Hz, 2H), 2.92 (t, J=7.6 Hz, 2H), 2.84-2.75 (m, 1H), 1.72-1.59 (m, 4H), 1.51-1.39 (m, 4H), 0.70-0.59 (m, 2H), 0.59-0.47 (m, 2H). LCMS (ESI, +ve mode) expected m/z for [M+H]⁺ 465.25, found 465.30.

### Example 158

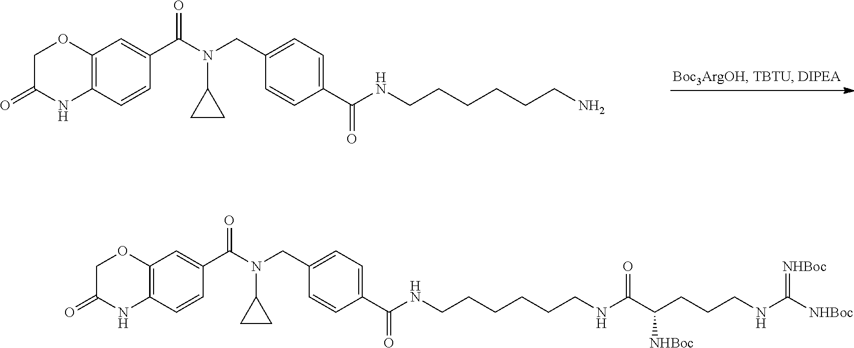

To a 2-dram vial was added (Z)—N2,Nw,Nw′-tris(tert-butoxycarbonyl)-L-arginine (5.2 mg 1.1 Eq, 11 μmol), 2-(1H-benzo[d][1,2,3]triazol-1-yl)-1,1,3,3-tetramethylisouronium tetrafluoroborate (4.2 mg, 1.3 Eq, 13 μmol), DIPEA (4.3 mg, 5.7 μL, 3.3 Eq, 33 μmol), and DMF (0.2 mL). The reaction was left to stir at room temperature for 30 minutes. To the vial was then added N-(4-((6-aminohexyl)carbamoyl)benzyl)-N-cyclopropyl-3-oxo-3,4-dihydro-2H-benzo[b][1,4]oxazine-7-carboxamide 2,2,2-trifluoroacetate (5.8 mg, 1 Eq, 10 μmol) dissolved in DMF (0.2 mL), and the reaction was left to stir overnight. The next day, the reaction was quenched with 4 mL of distilled water and extracted 3 times with 4 mL portions of ethyl acetate. The combined organic layers were washed twice with water, once with saturated sodium bicarbonate, and once with brine, then dried by passage through a phase separator and concentrated to a clear residue. Normal phase chromatography over silica (0-10% methanol in DCM) provided Product V (10.37 mg, 11.26 μmol, 1.1e+2%) as a clear residue. ¹H NMR (400 MHz, Chloroform-d) δ 7.79 (d, J=8.3 Hz, 2H), 7.38 (d, J=7.8 Hz, 2H), 7.16-7.10 (m, 2H), 7.01-6.95 (m, 1H), 6.84 (d, J=8.0 Hz, 1H), 6.51 (d, J=5.8 Hz, 1H), 5.99 (d, J=6.9 Hz, 1H), 4.75 (s, 2H), 4.62 (s, 2H), 4.32-4.23 (m, 1H), 4.02-3.91 (m, 1H), 3.74-3.64 (m, 1H), 3.41 (q, J=6.7 Hz, 2H), 3.38-3.28 (m, 1H), 3.17 (dd, J=13.1, 6.5 Hz, 1H), 2.60 (tt, J=7.0, 4.0 Hz, 1H), 1.83-1.70 (m, 3H), 1.65-1.53 (m, 4H), 1.50 (s, 9H), 1.49 (s, 9H), 1.44 (s, 9H), 1.42-1.29 (m, 5H), 0.65-0.55 (m, 2H), 0.55-0.45 (m, 2H). LCMS (ESI, +ve mode) expected m/z for [M+H]⁺ 921.51, found 921.40.

### Example 159

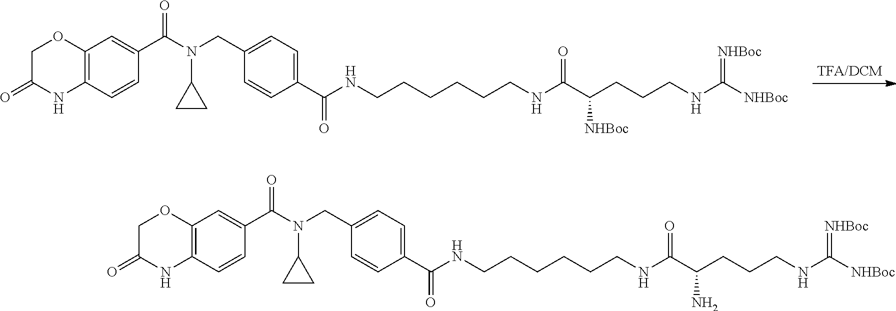

To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((6-(2-amino-5-guanidinopentanamido)hexyl)carbamoyl)benzyl)-N-cyclopropyl-3-oxo-3,4-dihydro-2H-benzo[b][1,4]oxazine-7-carboxamide bis(2,2,2-trifluoroacetate) (3.63 mg, 4.28 μmol, 78.2%) as a white solid. ¹H NMR (400 MHz, Methanol-d₄) δ 7.81 (d, J=8.2 Hz, 2H), 7.43 (d, J=7.9 Hz, 2H), 7.19 (d, J=8.2 Hz, 1H), 7.16 (s, 1H), 6.96 (d, J=8.0 Hz, 1H), 4.80 (s, 2H), 4.62 (s, 2H), 3.82 (t, J=6.6 Hz, 1H), 3.42-3.36 (m, 2H), 3.29-3.19 (m, 4H), 2.86-2.76 (m, 1H), 1.95-1.82 (m, 2H), 1.71-1.52 (m, 6H), 1.47-1.37 (m, 4H), 0.70-0.60 (m, 2H), 0.59-0.50 (m, 2H). LCMS (ESI, +ve mode) expected m/z for [M+H]⁺ 621.35, found 621.30; expected m/z for [M+2H]²⁺ 311.18, found 311.30.

### Example 160

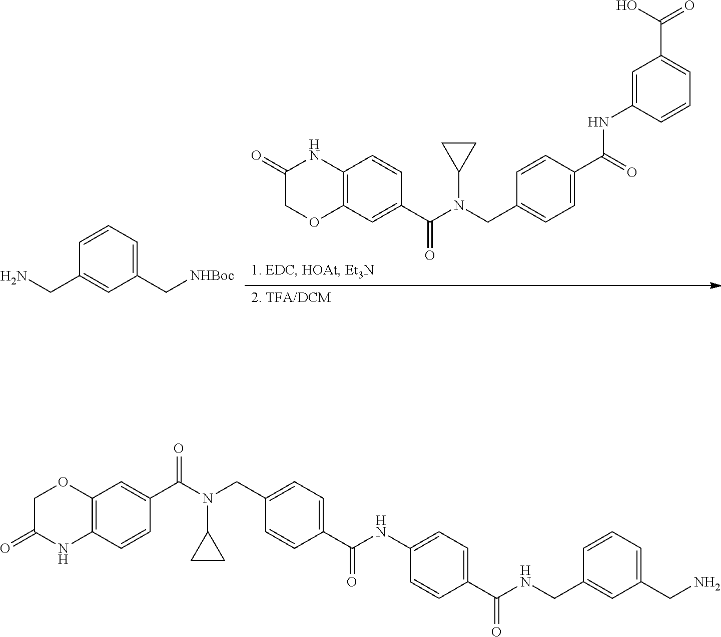 [SMILES: CC(C)(C)OC(=O)NCc1cccc(CN)c1 ; O=C1COc2cc(C(=O)N(Cc3ccc(C(=O)Nc4cccc(C(=O)O)c4)cc3)C3CC3)ccc2N1 ; NCc1cccc(CNC(=O)c2ccc(NC(=O)c3ccc(CN(C(=O)c4ccc5c(c4)OCC(=O)N5)C4CC4)cc3)cc2)c1]

To a 2-dram vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt(21 mg, 1.5 Eq, 0.15 mmol), and DMF (0.5 mL). The reaction was left to stir at room temperature for 30 minutes. To the vial was then added tert-butyl (3-(aminomethyl)benzyl)carbamate (29 mg, 1.2 Eq, 0.12 mmol), followed by triethylamine (31 mg, 43 μL, 3 Eq, 0.31 mmol), and the reaction was left to stir overnight.

The next day, the reaction was quenched with water and extracted 3 times with ethyl acetate. The combined organic fractions were washed once with 1 M KHSO₄, once with water, once with saturated sodium bicarbonate, and once with brine, then dried by passage through a phase separator and concentrated to a yellow residue. The residue was dissolved in 1.5 mL of 20% TFA and stirred overnight. The reaction was concentrated, co-evaporated with methanol, and purified by reverse phase chromatography (10-100% methanol in water+0.1% TFA) to provide N-(4-((4-((3-(aminomethyl)benzyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (19 mg, 26 μmol, 26%) as a white solid. ¹H NMR (400 MHz, Methanol-d₄) δ 7.95 (d, J=8.3 Hz, 2H), 7.93-7.83 (m, 4H), 7.53-7.47 (m, 2H), 7.47-7.45 (m, 1H), 7.45-7.41 (m, 2H), 7.35 (td, J=4.6, 1.8 Hz, 1H), 7.20 (dd, J=8.1, 1.7 Hz, 1H), 7.17 (d, J=1.7 Hz, 1H), 6.96 (d, J=8.0 Hz, 1H), 4.82 (s, 2H), 4.61 (s, 2H), 4.61 (s, 2H), 4.11 (s, 2H), 2.89-2.77 (m, 1H), 0.72-0.60 (m, 2H), 0.60-0.50 (m, 2H). LCMS (ESI, +ve mode) expected m/z for [M+H]⁺ 604.26, found 604.20.

### Example 161

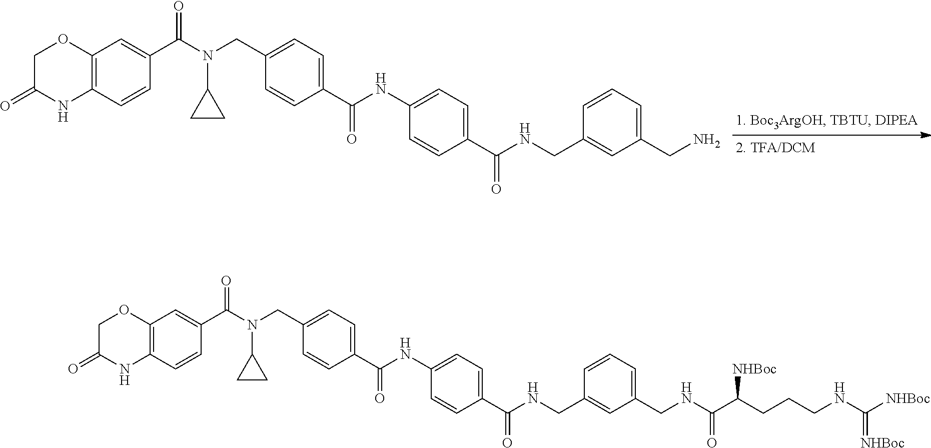

To a 2-dram vial was added (Z)—N2,Nw,Nw′-tris(tert-butoxycarbonyl)-L-arginine (7.3 mg 1.1 Eq, 15 μmol), 2-(1H-benzo[d][1,2,3]triazol-1-yl)-1,1,3,3-tetramethylisouronium tetrafluoroborate (5.8 mg, 1.3 Eq, 18 μmol), N-ethyl-N-isopropylpropan-2-amine (5.9 mg, 8.0 μL, 3.3 Eq, 46 μmol), and DMF (0.5 mL). The reaction was left to stir at room temperature for 15 minutes. To the vial was then added N-(4-((4-((3-(aminomethyl)benzyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol), and the reaction was left to stir overnight.

The reaction was quenched with 4 mL of distilled water and extracted 3 times with 4 mL portions of ethyl acetate. The combined organic layers were washed twice with water, once with saturated sodium bicarbonate, and once with brine, then dried by passage through a phase separator and concentrated to a clear residue. To the residue was added 1 mL of 50% TFA in DCM and stirred overnight.

The volatiles were removed on the rotavap, and the residue co-evaporated with methanol before purification by reverse phase chromatography (10-100% methanol in water+0.1% TFA) and lyophilization to provide (S)—N-(4-((4-((3-((2-amino-5-guanidinopentanamido)methyl)benzyl) carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate) (10.44 mg, 10.57 μmol, 76%) as a white solid.

¹H NMR (400 MHz, Methanol-d₄) δ 7.96 (d, J=8.3 Hz, 2H), 7.92-7.83 (m, 4H), 7.50 (d, J=7.9 Hz, 2H), 7.36-7.27 (m, 3H), 7.25-7.15 (m, 3H), 6.96 (d, J=8.0 Hz, 1H), 4.83 (s, 2H), 4.62 (s, 2H), 4.58 (s, 2H), 4.52 (d, J=14.8 Hz, 1H), 4.34 (d, J=14.8 Hz, 1H), 3.85 (t, J=6.6 Hz, 1H), 3.18 (t, J=7.1 Hz, 2H), 2.88-2.80 (m, 1H), 1.96-1.80 (m, 2H), 1.60 (p, J=7.6 Hz, 2H), 0.72-0.61 (m, 2H), 0.61-0.51 (m, 2H). LCMS (ESI, +ve mode) expected m/z for [M+H]⁺ 760.36, found 760.30; expected m/z for [M+2H]²⁺ 380.68, found 380.75.

### Example 162

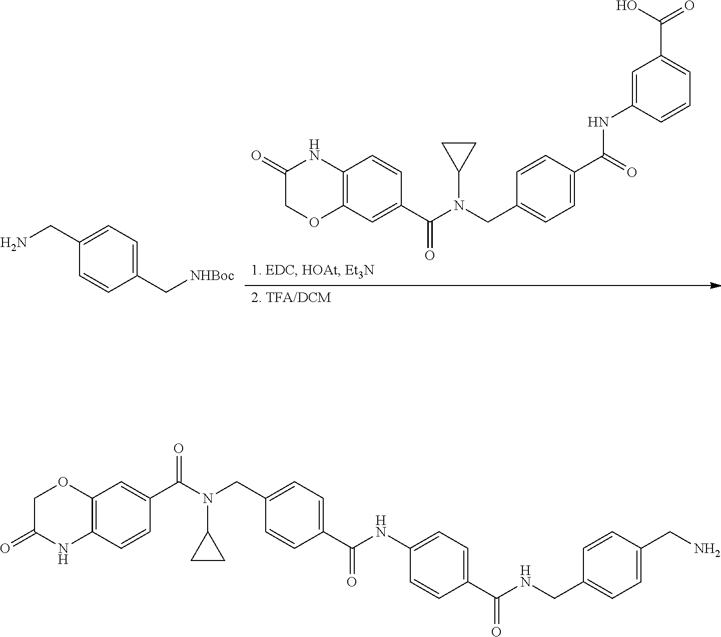 [SMILES: CC(C)(C)OC(=O)NCc1ccc(CN)cc1 ; O=C1COc2cc(C(=O)N(Cc3ccc(C(=O)Nc4cccc(C(=O)O)c4)cc3)C3CC3)ccc2N1 ; NCc1ccc(CNC(=O)c2ccc(NC(=O)c3ccc(CN(C(=O)c4ccc5c(c4)OCC(=O)N5)C4CC4)cc3)cc2)cc1]

To a 2-dram vial was added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), EDC (30 mg, 1.5 Eq, 0.15 mmol), HOAt (21 mg, 1.5 Eq, 0.15 mmol), and DMF (0.5 mL). The reaction was left to stir at room temperature for 30 minutes. To the vial was then added tert-butyl (4-(aminomethyl)benzyl)carbamate (29 mg, 1.2 Eq, 0.12 mmol), followed by triethylamine (31 mg, 43 μL, 3 Eq, 0.31 mmol), and the reaction was left to stir overnight.

The reaction was poured into water, forming a voluminous white precipitate that was collected by centrifugation. The pellet was washed once more with water before air-drying. The solids were taken up in 95% ethanol and concentrated into a vial, to which 2 mL of 20% TFA in DCM was added and stirred overnight. The volatiles were removed on the rotavap and the residue purified by reverse phase chromatography (10-100% methanol in water+0.10% TFA). ¹H NMR (400 MHz, Methanol-d₄) δ 7.95 (d, J=8.1 Hz, 2H), 7.92-7.81 (m, 4H), 7.54-7.47 (m, 2H), 7.47-7.39 (m, 4H), 7.20 (d, J=8.5 Hz, 1H), 7.17 (s, 1H), 6.96 (d, J=8.0 Hz, 1H), 4.83 (s, 2H), 4.62 (s, 2H), 4.60 (s, 2H), 4.10 (s, 2H), 2.88-2.77 (m, 1H), 0.71-0.60 (m, 2H), 0.60-0.48 (m, 2H). LCMS (ESI, +ve mode) expected m/z for [M+H]⁺ 604.26, found 604.20.

### Example 163

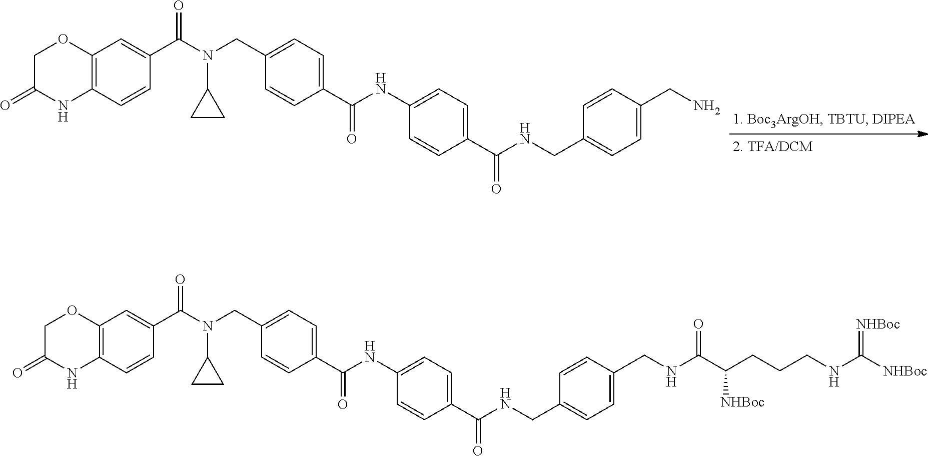

To a 2-dram vial were added Boc3-Arg-OH (7.3 mg, 1.1 Eq, 15 μmol), 2-(1H-benzo[d][1,2,3]triazol-1-yl)-1,1,3,3-tetramethylisouronium tetrafluoroborate (5.8 mg, 1.3 Eq, 18 μmol), DIPEA (5.9 mg, 8.0 μL, 3.3 Eq, 46 μmol), and dmf (0.5 mL). The reaction was left to stir at room temperature for 15 minutes. To the vial was then added N-(4-((4-((4-(aminomethyl)benzyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. To the residue was added 50% TFA in DCM, which was stirred until disappearance of starting material as monitored by LCMS. The reaction was concentrated, co-evaporated with methanol, and purified by reverse phase chromatography (10-100% methanol in water+0.10% TFA) to provide (S)—N-(4-((4-((4-((2-amino-5-guanidinopentanamido)methyl)benzyl)carbamoyl)phenyl) carbamoyl)benzyl)-N-cyclopropyl-3-oxo-3,4-dihydro-2H-benzo[b][1,4]oxazine-7-carboxamide bis(2,2,2-trifluoroacetate) (8.65 mg, 8.76 μmol, 63%) as a white solid. ¹H NMR (400 MHz, Methanol-d₄) δ 7.96 (d, J=8.3 Hz, 2H), 7.90-7.83 (m, 4H), 7.50 (d, J=7.9 Hz, 2H), 7.35 (d, J=8.2 Hz, 2H), 7.30 (d, J=8.2 Hz, 2H), 7.20 (d, J=8.6 Hz, 1H), 7.17 (d, J=1.7 Hz, 1H), 6.96 (d, J=8.1 Hz, 1H), 4.83 (s, 2H), 4.62 (s, 2H), 4.56 (s, 2H), 4.48 (d, J=14.7 Hz, 1H), 4.35 (d, J=14.7 Hz, 1H), 3.86 (t, J=6.5 Hz, 1H), 3.17 (t, J=7.1 Hz, 2H), 2.87-2.80 (m, 1H), 1.88 (hept, J=7.0 Hz, 2H), 1.59 (p, J=7.7 Hz, 2H), 0.71-0.61 (m, 2H), 0.61-0.52 (m, 2H). LCMS (ESI, +ve mode) expected m/z for [M+H]⁺ 760.36, found 760.25; expected m/z for [M+2H]²+380.68, found 380.75.

### Example 164

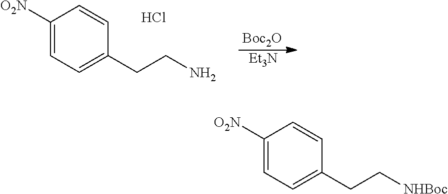 [SMILES: "Boc2O" ; "Et3N" ; NCCc1ccc([N+](=O)[O-])cc1 ; Cl ; CC(C)(C)OC(=O)NCCc1ccc([N+](=O)[O-])cc1]

To a flask was added 2-(4-nitrophenyl)ethan-1-amine hydrochloride (200 mg, 1 Eq, 987 μmol) and DCM (5 mL), then triethylamine (110 mg, 151 μL, 1.1 Eq, 1.09 mmol) was added. To the stirring solution was added boc2o (237 mg, 249 μL, 1.1 Eq, 1.09 mmol), and it was stirred overnight. The next day, the organic layer was washed once with saturated ammonium chloride, once with water, once with saturated sodium bicarbonate, and once with brine, then dried over anhydrous sodium sulfate, filtered, and concentrated to a clear oil. Purification by normal phase chromatography over silica gel (0-25% ethyl acetate in hexanes) provided tert-butyl (4-nitrophenethyl)carbamate (224.4 mg, 842.7 μmol, 85.4%) as a clear residue. ¹H NMR (400 MHz, Chloroform-d) δ 8.17 (d, J=8.7 Hz, 2H), 7.36 (d, J=8.6 Hz, 2H), 4.56 (s, 1H), 3.41 (q, J=6.8 Hz, 2H), 2.92 (t, J=7.0 Hz, 2H), 1.43 (s, 9H).

### Example 165

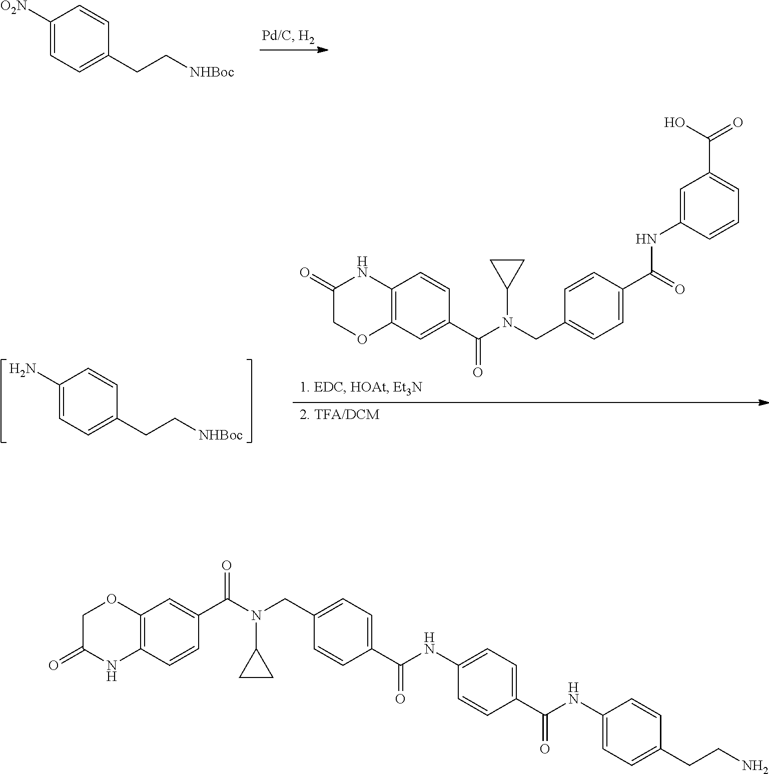

To a flask was added tert-butyl (4-nitrophenethyl)carbamate (50 mg, 1.8 Eq, 0.19 mmol) and ethanol (1.5 mL). The atmosphere was cycled once to nitrogen, and left to stir under the N2 line for 15 minutes. Next, Pd/C (4 mg, 10% Wt, 0.04 Eq, 4 μmol) was added, and the atmosphere cycled again. Finally, the N2 line was removed and a balloon of hydrogen was attached. After 4 hours, TLC (3:1 hexanes/ethyl acetate) indicated complete conversion. The reaction was filtered through Celite and concentrated to a clear oil. Normal phase chromatography over silica (0-50% ethyl acetate in hexanes) yielded the aniline (37.8 mg, 15.8 μmol, 84%) as a clear oil that crystallized on standing. NMR is consistent with structure. ¹H NMR (400 MHz, Chloroform-d) δ 6.97 (d, J=8.1 Hz, 2H), 6.63 (d, J=8.3 Hz, 2H), 4.60-4.50 (m, 1H), 3.60 (s, 2H), 3.31 (q, J=6.8 Hz, 2H), 2.67 (t, J=7.0 Hz, 2H), 1.43 (s, 9H). ¹³C NMR (101 MHz, cdcl₃) δ 156.02, 144.90, 129.71, 128.94, 115.46, 79.19, 42.11, 35.35, 28.53.

To a 2-dram vial was added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), HOAt (21 mg, 1.5 Eq, 0.15 mmol), EDC (30 mg, 1.5 Eq, 0.15 mmol), and DMF (0.5 mL). The reaction was left to stir at room temperature for 15 minutes. To the vial was then added the aniline, followed by triethylamine (31 mg, 43 μL, 3 Eq, 0.31 mmol), and the reaction was left to stir overnight.

The reaction was poured into 14 mL of water, yielding a voluminous white precipitate that was collected by centrifugation. The pellet was washed once more with 14 mL of water and briefly air-dried. The solids were taken up in 95% ethanol and concentrated into a vial, and 2 mL of 20% TFA was added and the reaction stirred overnight.

The volatiles were removed on the rotavap, and the residue co-evaporated with methanol before purification by reverse phase chromatography (10-100% methanol in water+0.1% TFA) and lyophilization to yield N-(4-((4-((4-(2-aminoethyl)phenyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (25.2 mg, 35.1 μmol, 34%) as a white solid. ¹H NMR (400 MHz, Methanol-d₄) δ 8.01-7.94 (m, 4H), 7.94-7.87 (m, 2H), 7.69 (d, J=8.6 Hz, 2H), 7.56-7.46 (m, 2H), 7.30 (d, J=8.2 Hz, 2H), 7.21 (d, J=8.2 Hz, 1H), 7.19 (s, 1H), 6.97 (d, J=8.0 Hz, 1H), 4.63 (s, 2H), 3.19 (t, J=7.7 Hz, 2H), 2.96 (t, J=7.7 Hz, 2H), 2.89-2.79 (m, 1H), 0.72-0.62 (m, 2H), 0.62-0.50 (m, 2H). Note: 2H hidden under water peak. LCMS (ESI, +ve mode) expected m/z for [M+H]⁺ 604.26, found 604.20.

### Example 166

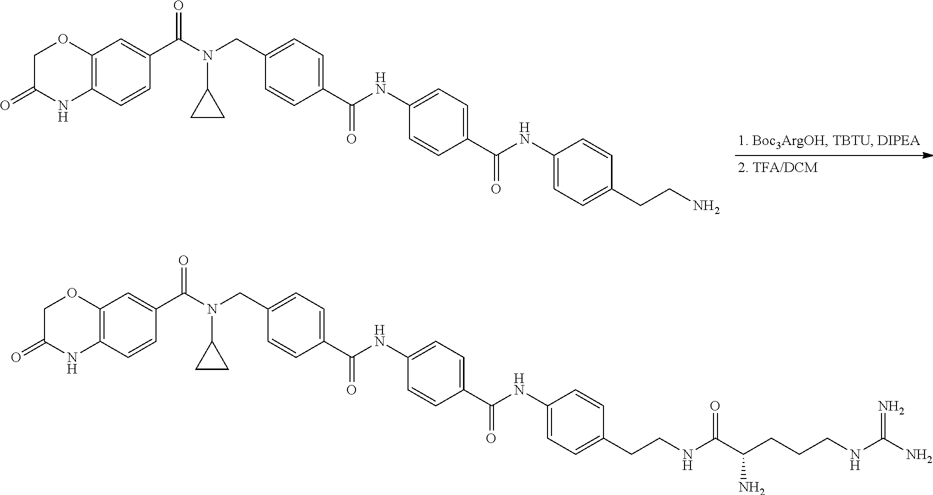 [SMILES: NCCc1ccc(NC(=O)c2ccc(NC(=O)c3ccc(CN(C(=O)c4ccc5c(c4)OCC(=O)N5)C4CC4)cc3)cc2)cc1 ; N=C(N)NCCC[C@H](N)C(=O)NCCc1ccc(NC(=O)c2ccc(NC(=O)c3ccc(CN(C(=O)c4ccc5c(c4)OCC(=O)N5)C4CC4)cc3)cc2)cc1]

To a 2-dram vial were added (Z)—N2,Nw,Nw′-tris(tert-butoxycarbonyl)-L-arginine (7.3 mg 1.1 Eq, 15 μmol), 2-(1H-benzo[d][1,2,3]triazol-1-yl)-1,1,3,3-tetramethylisouronium tetrafluoroborate (5.8 mg, 1.3 Eq, 18 μmol), DIPEA (5.9 mg, 8.0 μL, 3.3 Eq, 46 μmol), and dmf (0.5 mL). The reaction was left to stir at room temperature for 15 minutes. To the vial was then added N-(4-((4-((4-(2-aminoethyl)phenyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol) dissolved in dmf (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. To the residue was added 50% TFA in DCM, which was stirred until disappearance of starting material as monitored by LCMS. The reaction was concentrated, co-evaporated with methanol, and purified by reverse phase chromatography (10-100% methanol in water+0.10% TFA) to provide (S)—N-(4-((4-((4-(2-(2-amino-5-guanidinopentanamido)ethyl)phenyl)carbamoyl)phenyl) carbamoyl)benzyl)-N-cyclopropyl-3-oxo-3,4-dihydro-2H-benzo[b][1,4]oxazine-7-carboxamide bis(2,2,2-trifluoroacetate) (9.53 mg, 9.65 μmol, 69%) as a white solid. ¹H NMR (400 MHz, Methanol-d₄) δ 8.00-7.95 (m, 4H), 7.92-7.88 (m, 2H), 7.61 (d, J=8.5 Hz, 2H), 7.51 (d, J=7.9 Hz, 2H), 7.26 (d, J=8.5 Hz, 2H), 7.21 (d, J=8.5 Hz, 1H), 7.18 (d, J=1.7 Hz, 1H), 6.97 (d, J=8.1 Hz, 1H), 4.84 (s, 2H), 4.63 (s, 2H), 3.79 (t, J=6.4 Hz, 1H), 3.70 (dt, J=14.1, 7.3 Hz, 1H), 3.43 (dt, J=13.3, 6.6 Hz, 1H), 3.17 (t, J=7.0 Hz, 2H), 2.95-2.89 (m, 1H), 2.89-2.79 (m, 2H), 1.85-1.72 (m, 2H), 1.50 (p, J=7.7 Hz, 2H), 0.72-0.62 (m, 2H), 0.62-0.51 (m, 2H). LCMS (ESI, +ve mode) expected m/z for [M+H]⁺ 760.36, found 760.30; expected m/z for [M+2H]²⁺ 380.68, found 380.70.

### Example 167

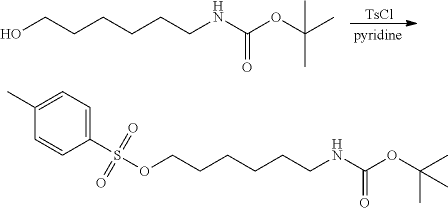

To a flask were added tert-butyl (6-hydroxyhexyl)carbamate (0.22 g, 1 Eq, 1.0 mmol), DCM (5 mL), and pyridine (0.16 g, 0.16 mL, 2 Eq, 2.0 mmol). The reaction was cooled in an ice bath and tosyl-C1 (0.29 g, 1.5 Eq, 1.5 mmol) was added. The reaction was allowed to come to room temperature with stirring overnight. The next day, the volatiles were removed on the rotovap and the residue was partitioned between water and ethyl acetate. The layers were separated, and the aqueous layer extracted twice more with ethyl acetate. The combined organic fractions were washed thrice with 0.5 M citric acid, once with water, once with saturated sodium bicarbonate, and once with brine, then dried over sodium sulfate, filtered, and concentrated to a residue. Normal phase chromatography over silica gel (0-40% ethyl acetate in hexanes) provided 6-((tert-butoxycarbonyl)amino)hexyl 4-methylbenzenesulfonate (329 mg, 886 μmol, 89%) as a clear residue. ¹H NMR (400 MHz, Chloroform-d) δ 7.70 (d, J=8.3 Hz, 2H), 7.27 (d, J=8.0 Hz, 2H), 4.71-4.59 (m, 1H), 3.93 (t, J=6.4 Hz, 2H), 2.97 (q, J=6.7 Hz, 2H), 2.37 (s, 3H), 1.60-1.49 (m, 2H), 1.42-1.28 (m, 11H), 1.28-1.12 (m, 4H). ¹³C NMR (101 MHz, Chloroform-d) δ 155.94, 144.68, 133.01, 129.78, 127.74, 78.78, 70.45, 40.24, 29.73, 28.62, 28.33, 25.99, 24.94, 21.51.

### Example 168

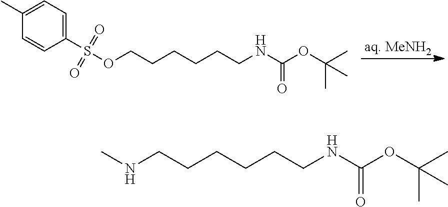

To a flask were added 6-((tert-butoxycarbonyl)amino)hexyl 4-methylbenzenesulfonate (100 mg, 1 Eq, 269 μmol), THF (1 mL), and finally aqueous methanamine (418 mg, 466 μL, 40% Wt, 20 Eq, 5.38 mmol). The solution was heated to reflux until consumption of the starting material. The reaction was cooled, diluted with water, and extracted three times with dichloromethane. The combined organic extracts were washed once with brine, dried over anhydrous sodium sulfate, filtered, and concentrated to yield tert-butyl (6-(methylamino)hexyl)carbamate (38.2 mg, 166 μmol, 61.6%) as a residue that was used without further purification. ¹H NMR (400 MHz, Chloroform-d) δ 4.60 (s, 1H), 3.07 (q, J=6.7 Hz, 2H), 2.54 (t, J=7.2 Hz, 2H), 2.40 (s, 3H), 1.50-1.42 (m, 4H), 1.41 (s, 9H), 1.36-1.23 (m, J=2.7, 1.9 Hz, 4H). ¹³C NMR (101 MHz, Chloroform-d) δ 156.11, 79.07, 57.84, 51.97, 36.40, 30.09, 29.68, 28.52, 27.03, 26.75. LCMS (ESI, +ve mode) expected m/z for [M+H]+ 231.21, found 231.20.

### Example 169

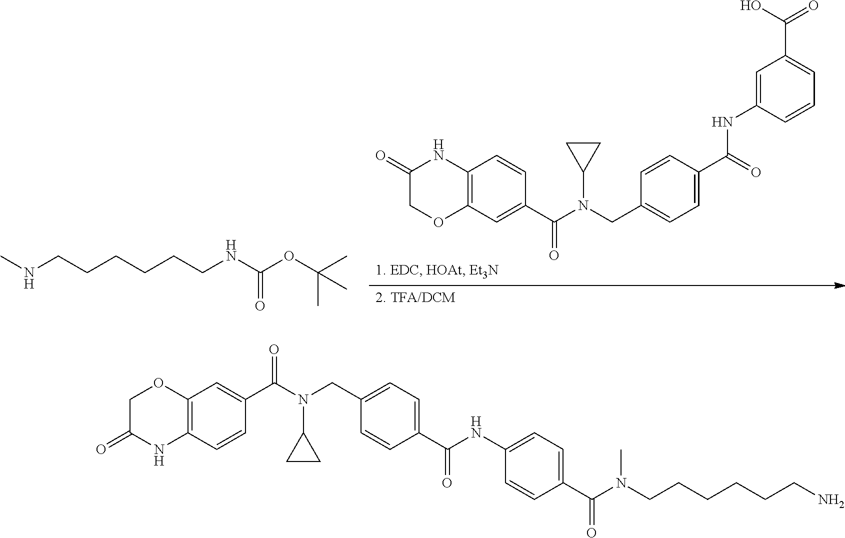

To a 2-dram vial were added 4-(4-((N-cyclopropyl-3-oxo-3,4-dihydro-2H-benzo[b][1,4]oxazine-7-carboxamido)methyl)benzamido)benzoic acid (80 mg, 1 Eq, 0.16 mmol), EDC (47 mg, 1.5 Eq, 0.25 mmol), HOAt (34 mg, 1.5 Eq, 0.25 mmol), and DMF (0.5 mL). The reaction was left to stir at room temperature for 30 minutes. To the vial was then added tert-butyl (6-(methylamino)hexyl)carbamate (38 mg, 1 Eq, 0.16 mmol) dissolved in DMF (0.5 mL), followed by triethylamine (50 mg, 69 μL, 3 Eq, 0.49 mmol), and the reaction was left to stir overnight.

The next day, the reaction was quenched with water and extracted 3 times with ethyl acetate. The combined organic fractions were washed once with 1 M KHSO4, once with water, once with saturated sodium bicarbonate, and once with brine, then dried by passage through a phase separator and concentrated to a yellow residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-(4-((4-((6-aminohexyl)(methyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (35.9 mg, 50.4 μmol, 31%) as a white solid. ¹H NMR (400 MHz, Methanol-d₄) δ 7.95 (d, J=8.2 Hz, 2H), 7.83 (d, J=8.1 Hz, 2H), 7.49 (d, J=7.7 Hz, 2H), 7.43 (d, J=7.7 Hz, 2H), 7.20 (dd, J=8.0, 1.7 Hz, 1H), 7.17 (d, J=1.7 Hz, 1H), 6.96 (d, J=8.0 Hz, 1H), 4.82 (s, 2H), 4.61 (s, 2H), 3.56 (s, 1H), 3.36 (d, J=6.9 Hz, 1H), 3.05 (d, J=16.0 Hz, 3H), 2.93 (d, J=7.3 Hz, 1H), 2.84 (s, 2H), 1.65 (d, J=43.1 Hz, 4H), 1.47 (s, 2H), 1.22 (s, 2H), 0.64 (d, J=6.7 Hz, 2H), 0.55 (s, 2H). LCMS (ESI, +ve mode) expected m/z for [M+H]⁺ 598.30, found 598.25.

### Example 170

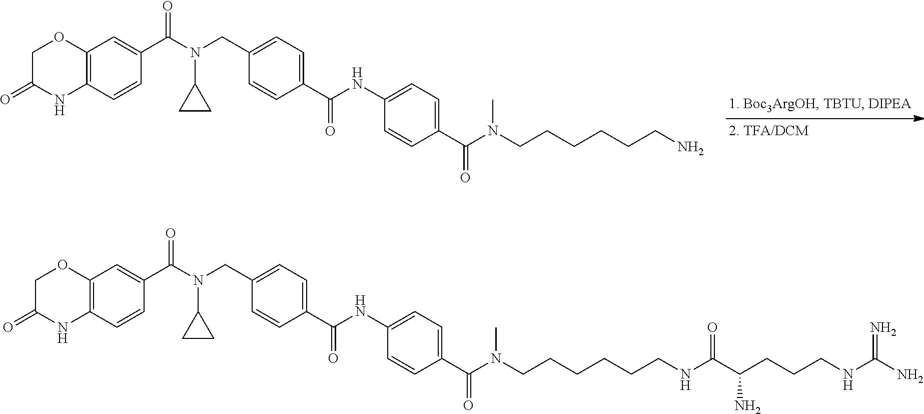 [SMILES: CN(CCCCCCN)C(=O)c1ccc(NC(=O)c2ccc(CN(C(=O)c3ccc4c(c3)OCC(=O)N4)C3CC3)cc2)cc1 ; CN(CCCCCCNC(=O)[C@@H](N)CCCNC(=N)N)C(=O)c1ccc(NC(=O)c2ccc(CN(C(=O)c3ccc4c(c3)OCC(=O)N4)C3CC3)cc2)cc1]

To a 2-dram vial was added (Z)—N2,Nw,Nw′-tris(tert-butoxycarbonyl)-L-arginine (7.3 mg 1.1 Eq, 15 μmol), TBTU (5.9 mg, 1.3 Eq, 18 μmol), DIPEA (6.0 mg, 8.1 μL, 3.3 Eq, 46 μmol), and dmf (0.5 mL). The reaction was left to stir at room temperature for 15 minutes. To the vial was then added N-(4-((4-((6-aminohexyl)(methyl)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol) and the reaction was left to stir overnight.

The reaction was quenched with 4 mL of distilled water and extracted 3 times with 4 mL portions of ethyl acetate. The combined organic layers were washed twice with water, once with saturated sodium bicarbonate, and once with brine, then dried by passage through a phase separator and concentrated to a clear residue. To the residue was added 1 mL of 50% TFA in DCM, and the reaction was stirred overnight.

The volatiles were removed on the rotavap, and the residue co-evaporated with methanol before purification by reverse phase chromatography (10-100% methanol in water+0.1% TFA) and lyophilization to provide (S)—N-(4-((4-((6-(2-amino-5-guanidinopentanamido)hexyl)(methyl) carbamoyl)phenyl)carbamoyl)benzyl)-N-cyclopropyl-3-oxo-3,4-dihydro-2H-benzo[b][1,4]oxazine-7-carboxamide bis(2,2,2-trifluoroacetate) (11.14 mg, 11.34 μmol, 81%) as a white solid. ¹H NMR (400 MHz, Methanol-d₄) δ 7.96 (d, J=8.2 Hz, 2H), 7.83 (d, J=8.5 Hz, 2H), 7.50 (d, J=7.9 Hz, 2H), 7.47-7.39 (m, 2H), 7.21 (d, J=8.2 Hz, 1H), 7.17 (d, J=1.7 Hz, 1H), 6.97 (d, J=8.1 Hz, 1H), 4.83 (s, 2H), 4.62 (s, 2H), 3.83 (t, J=6.6 Hz, 1H), 3.62-3.51 (m, 1H), 3.42-3.34 (m, 1H), 3.29-3.26 (m, 1H), 3.22 (t, J=7.0 Hz, 2H), 3.19-3.14 (m, 1H), 3.10-3.00 (m, 3H), 2.89-2.80 (m, 1H), 1.94-1.82 (m, 2H), 1.76-1.54 (m, 5H), 1.51-1.41 (m, 3H), 1.26-1.16 (m, 2H), 0.71-0.61 (m, 2H), 0.61-0.50 (m, 2H). LCMS (ESI, +ve mode) expected m/z for [M+H]⁺ 754.40, found 754.30; expected m/z for [M+2H]²⁺ 377.70, found 377.80.

### Example 171

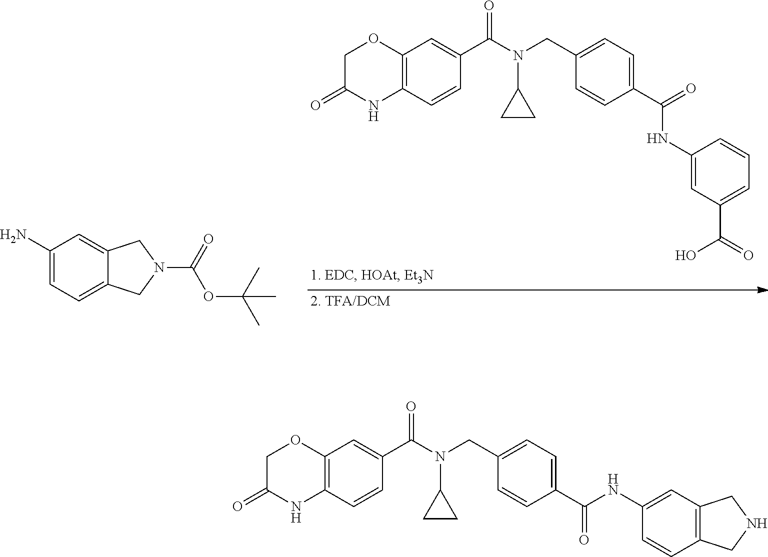 [SMILES: CC(C)(C)OC(=O)N1Cc2ccc(N)cc2C1 ; O=C1COc2cc(C(=O)N(Cc3ccc(C(=O)Nc4cccc(C(=O)O)c4)cc3)C3CC3)ccc2N1 ; O=C1COc2cc(C(=O)N(Cc3ccc(C(=O)Nc4ccc5c(c4)CNC5)cc3)C3CC3)ccc2N1]

To a 2-dram vial was added 4-((N-cyclopropyl-3-oxo-3,4-dihydro-2H-benzo[b][1,4]oxazine-7-carboxamido)methyl)benzoic acid (100 mg, 1 Eq, 273 μmol), EDC (78.5 mg, 1.5 Eq, 409 μmol), HOAt (55.7 mg, 1.5 Eq, 409 μmol), and DMF (0.6 mL). The reaction was left to stir at room temperature for 30 minutes. To the vial was then added Tert-Butyl 5-aminoisoindoline-2-carboxylate (76.7 mg 1.2 Eq, 328 μmol) dissolved in DMF (0.6 mL), followed by triethylamine (82.9 mg, 114 μL, 3 Eq, 819 μmol), and the reaction was left to stir overnight.

The next day, the reaction was quenched with 4 mL of distilled water and extracted 3 times with 4 mL portions of ethyl acetate. The combined organic layers were washed once with 1M KHSO4, once with water, once with saturated sodium bicarbonate, and once with brine, then dried by passage through a phase separator and concentrated to an orange residue. To the residue was added 20% TFA in DCM, which was stirred until disappearance of starting material as monitored by LCMS. The reaction was concentrated, co-evaporated with methanol, and purified by reverse phase chromatography (10-100% methanol in water+0.1% TFA) to provide N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (116.9 mg, 196.0 μmol, 71.80%) as a white solid. ¹H NMR (400 MHz, Methanol-d₄) δ 7.96-7.89 (m, 2H), 7.87 (d, J=1.9 Hz, 1H), 7.65 (dd, J=8.3, 2.0 Hz, 1H), 7.51-7.39 (m, 2H), 7.36 (d, J=8.4 Hz, 1H), 7.17 (dd, J=8.1, 1.7 Hz, 1H), 7.13 (d, J=1.7 Hz, 1H), 6.94 (d, J=8.1 Hz, 1H), 4.79 (s, 2H), 4.61 (s, 2H), 4.59 (s, 2H), 4.58 (s, 2H), 2.87-2.72 (m, 1H), 0.69-0.57 (m, 2H), 0.57-0.43 (m, 2H). ¹³C NMR (101 MHz, cd₃od) δ 173.87, 168.56, 168.47, 167.37, 167.27, 162.25, 161.88, 144.50, 143.46, 140.67, 136.34, 134.94, 133.21, 131.19, 129.79, 129.10, 128.78, 124.24, 123.16, 122.64, 116.83, 116.73, 116.39, 68.05, 51.89, 51.63, 33.70, 10.54. LCMS (ESI, +ve mode) expected m/z for [M+H]⁺ 483.20, found 483.10.

### Example 172

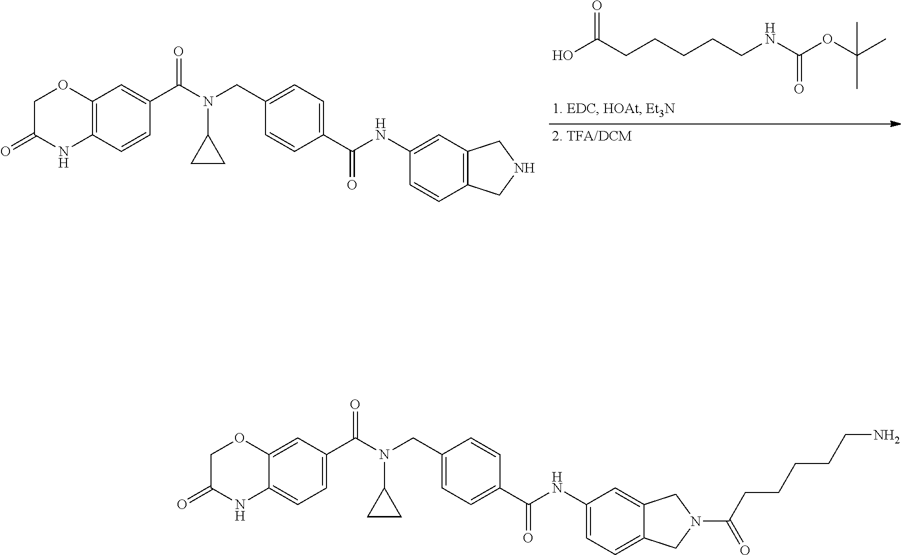 [SMILES: O=C1COc2cc(C(=O)N(Cc3ccc(C(=O)Nc4ccc5c(c4)CNC5)cc3)C3CC3)ccc2N1 ; CC(C)(C)OC(=O)NCCCCCC(=O)O ; NCCCCCC(=O)N1Cc2ccc(NC(=O)c3ccc(CN(C(=O)c4ccc5c(c4)OCC(=O)N5)C4CC4)cc3)cc2C1]

To a 2-dram vial was added 6-((tert-butoxycarbonyl)amino)hexanoic acid (25 mg, 1.3 Eq, 0.11 mmol), EDC (32 mg, 2 Eq, 0.17 mmol), HOAt (23 mg, 2 Eq, 0.17 mmol), and 200 μL DMF. The reaction was left to stir at room temperature for 30 minutes. To the vial was then added N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (50 mg, 1 Eq, 84 μmol) dissolved in 300 μL DMF, followed by triethylamine (34 mg, 47 μL, 4 Eq, 0.34 mmol), and the reaction was left to stir overnight.

The reaction was quenched with 4 mL of distilled water and extracted 3 times with 4 mL portions of ethyl acetate. The combined organic layers were washed once with 0.5 M citric acid, once with water, once with saturated sodium bicarbonate, and once with brine, then dried by passage through a phase separator and concentrated to a clear residue. To the residue was added 2 mL of 20% TFA in DCM, and the reaction was stirred at room temperature for 6 hours. The volatiles were removed on the rotavap, and the residue purified by reverse phase chromatography (10-100% methanol in water+0.1% TFA) and lyophilized to yield N-(4-((2-(6-aminohexanoyl)isoindolin-5-yl)carbamoyl)benzyl)-N-cyclopropyl-3-oxo-3,4-dihydro-2H-benzo[b][1,4]oxazine-7-carboxamide 2,2,2-trifluoroacetate (55 mg, 77 μmol, 92%) as a white solid. ¹H NMR (400 MHz, Methanol-d₄) δ 7.93 (d, J=8.3 Hz, 2H), 7.75 (dd, J=23.5, 1.9 Hz, 1H), 7.54 (dd, J=8.3, 2.0 Hz, 1H), 7.51-7.42 (m, 2H), 7.29 (dd, J=8.4, 4.5 Hz, 1H), 7.18 (d, J=8.3 Hz, 1H), 7.15 (d, J=1.8 Hz, 1H), 6.96 (d, J=0.8 Hz, 1H), 4.86-4.77 (m, 4H), 4.70 (d, J=8.2 Hz, 2H), 4.60 (s, 2H), 2.94 (t, J=7.6 Hz, 2H), 2.87-2.76 (m, 1H), 2.47 (t, J=7.2 Hz, 2H), 1.70 (dtd, J=15.2, 7.5, 3.7 Hz, 4H), 1.48 (td, J=8.4, 5.2 Hz, 2H), 0.70-0.58 (m, 2H), 0.58-0.46 (m, 2H). ¹³C NMR (101 MHz, Methanol-d₄) δ 174.20, 168.45, 167.31, 144.61, 143.44, 139.76, 139.63, 138.35, 137.94, 135.18, 133.62, 133.24, 129.92, 129.09, 128.84, 124.21, 124.09, 123.22, 121.97, 121.85, 116.90, 116.71, 116.60, 116.51, 68.13, 53.76, 53.40, 53.20, 52.84, 40.50, 34.57, 34.55, 28.32, 27.01, 25.05, 10.37. LCMS (ESI, +ve mode) expected m/z for [FORMULA] [M+H] 596.29, found 596.20.

### Example 173

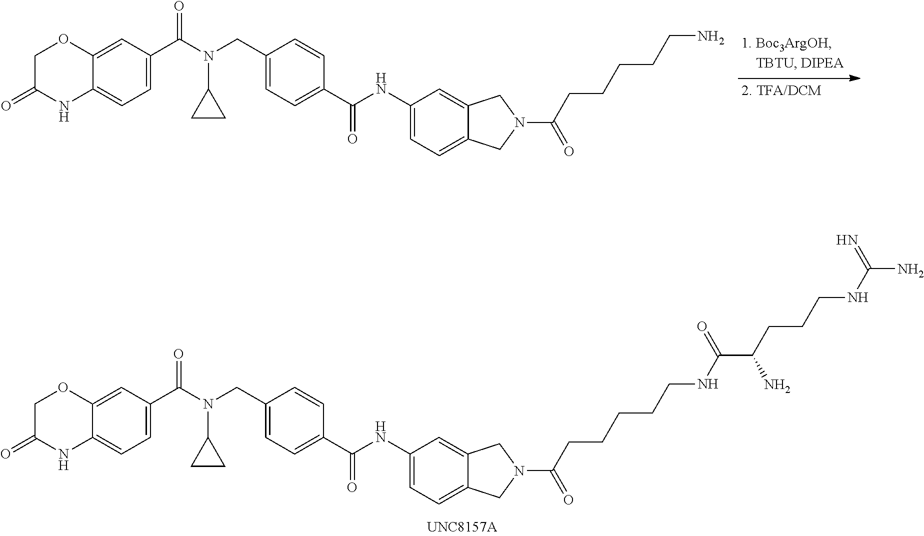 [SMILES: NCCCCCC(=O)N1Cc2ccc(NC(=O)c3ccc(CN(C(=O)c4ccc5c(c4)OCC(=O)N5)C4CC4)cc3)cc2C1 ; N=C(N)NCCC[C@H](N)C(=O)NCCCCCC(=O)N1Cc2ccc(NC(=O)c3ccc(CN(C(=O)c4ccc5c(c4)OCC(=O)N5)C4CC4)cc3)cc2C1]

To a 2-dram vial were added Boc3-Arg-OH (7.4 mg, 1.1 Eq, 15 μmol), 2-(1H-benzo[d][1,2,3]triazol-1-yl)-1,1,3,3-tetramethylisouronium tetrafluoroborate (5.9 mg, 1.3 Eq, 18 μmol), DIPEA (6.0 mg, 8.1 μL, 3.3 Eq, 46 μmol), and dmf (0.5 mL). The reaction was left to stir at room temperature for 15 minutes. To the vial was then added N-(4-((2-(6-aminohexanoyl)isoindolin-5-yl)carbamoyl)benzyl)-N-cyclopropyl-3-oxo-3,4-dihydro-2H-benzo[b][1,4]oxazine-7-carboxamide 2,2,2-trifluoroacetate (10 mg, 1 Eq, 14 μmol) dissolved in dmf (0.5 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((2-(6-(2-amino-5-guanidinopentanamido)hexanoyl)isoindolin-5-yl)carbamoyl) benzyl)-N-cyclopropyl-3-oxo-3,4-dihydro-2H-benzo[b][1,4]oxazine-7-carboxamide bis(2,2,2-trifluoroacetate) (9.70 mg, 9.90 μmol, 70%) as a white solid. ¹H NMR (400 MHz, Methanol-d₄) δ 7.95 (d, J=8.2 Hz, 2H), 7.80 (d, J=27.0 Hz, 1H), 7.61-7.51 (m, 1H), 7.52-7.46 (m, 2H), 7.33 (dd, J=8.0, 2.7 Hz, 1H), 7.20 (d, J=8.2 Hz, 1H), 7.17 (d, J=1.8 Hz, 1H), 6.96 (d, J=8.0 Hz, 1H), 4.91 (s, 2H), 4.83 (s, 2H), 4.75 (d, J=11.1 Hz, 2H), 4.62 (s, 2H), 3.84 (t, J=6.6 Hz, 1H), 3.37-3.32 (m, 1H), 3.28-3.20 (m, 3H), 2.87-2.79 (m, 1H), 2.49 (t, J=7.2 Hz, 2H), 1.95-1.83 (m, 2H), 1.76-1.56 (m, 6H), 1.51-1.41 (m, 2H), 0.71-0.61 (m, 2H), 0.61-0.50 (m, 2H). LCMS (ESI, +ve mode) expected m/z for [M+H]⁺ 752.39, found 752.30; expected m/z for [M+2H]²⁺ 376.70, found 376.80.

### Example 174

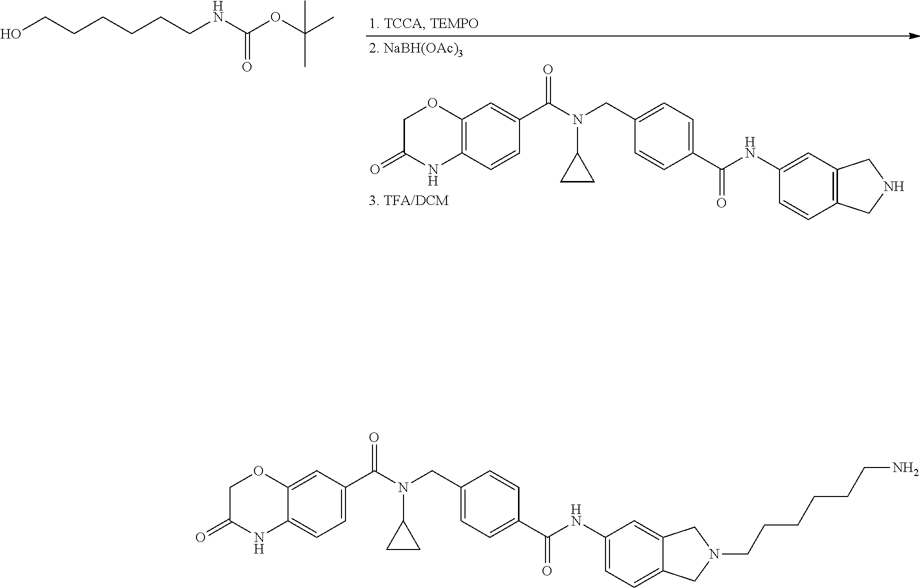 [SMILES: CC(C)(C)OC(=O)NCCCCCCO ; O=C1COc2cc(C(=O)N(Cc3ccc(C(=O)Nc4ccc5c(c4)CNC5)cc3)C3CC3)ccc2N1 ; NCCCCCCN1Cc2ccc(NC(=O)c3ccc(CN(C(=O)c4ccc5c(c4)OCC(=O)N5)C4CC4)cc3)cc2C1]

To a flask was added tert-butyl (6-hydroxyhexyl)carbamate (0.11 g, 6.0 Eq, 0.50 mmol) and DCM (1 mL). The flask was cooled in an ice bath, and 1,3,5-trichloro-1,3,5-triazinane-2,4,6-trione (122 mg, 6.3 Eq, 525 μmol) was added followed by TEMPO (0.8 mg, 0.06 Eq, 5 μmol). The reaction was stirred at 0° C. for 1 hour, until TLC (1:1 hexanes/ethyl acetate) showed complete conversion. The reaction was diluted with DCM and filtered through Celite, and the filtrate washed once with 1M HCl, once with water, once with saturated sodium bicarbonate, and once with brine, then dried over sodium sulfate and concentrated to a clear oil. The aldehyde was used immediately.

To a vial under nitrogen atmosphere was added N-cyclopropyl-N-(4-(isoindolin-5-ylcarbamoyl)benzyl)-3-oxo-3,4-dihydro-2H-benzo[b][1,4]oxazine-7-carboxamide 2,2,2-trifluoroacetate (50 mg, 1 Eq, 84 μmol) and sodium triacetoxyhydroborate (71 mg, 4 Eq, 0.34 mmol). The aldehyde was dissolved in anhydrous THF (2 mL) and added to the vial. The reaction was stirred under nitrogen atmosphere overnight.

The reaction was diluted with saturated sodium bicarbonate and extracted three times with ethyl acetate. The combined organic layers were washed twice with water, once with saturated sodium bicarbonate, and once with brine, then dried over sodium sulfate and concentrated to a yellowish residue. The residue was dissolved in 2.5 mL of 20% TFA in DCM and stirred for 6 hours. The volatiles were removed on the rotavap, and the residue co-evaporated with methanol before purification by reverse phase chromatography (10-50% methanol in water+0.1% TFA) to provide N-(4-((2-(6-aminohexyl)isoindolin-5-yl)carbamoyl)benzyl)-N-cyclopropyl-3-oxo-3,4-dihydro-2H-benzo[b][1,4]oxazine-7-carboxamide bis(2,2,2-trifluoroacetate)(33.4 mg, 41.2 μmol, 49%) as a white solid. ¹H NMR (400 MHz, Methanol-d₄) δ 7.94 (d, J=8.3 Hz, 2H), 7.92 (s, 1H), 7.65 (dd, J=8.3, 1.9 Hz, 1H), 7.49 (d, J=7.9 Hz, 2H), 7.39 (d, J=8.3 Hz, 1H), 7.20 (dd, J=8.1, 1.8 Hz, 1H), 7.17 (s, 1H), 6.96 (d, J=8.1 Hz, 1H), 4.82 (s, 2H), 4.62 (s, 2H), 4.56 (s, 2H), 3.49-3.40 (m, 2H), 2.95 (t, J=7.7 Hz, 2H), 2.87-2.78 (m, 1H), 1.90-1.79 (m, 2H), 1.75-1.65 (m, 2H), 1.55-1.44 (m, 4H), 0.71-0.60 (m, 2H), 0.60-0.49 (m, 2H). ¹³C NMR (101 MHz, Methanol-d₄) δ 168.63, 167.35, 144.66, 141.05, 135.76, 135.06, 130.53, 129.96, 129.12, 128.90, 124.33, 123.23, 122.80, 116.90, 116.72, 116.42, 68.15, 59.80, 59.57, 56.08, 40.51, 28.32, 27.02, 26.94, 26.59, 10.52. LCMS (ESI, +ve mode) expected m/z for [M+H]⁺ 582.31, found 582.25; expected m/z for [M+2H]²⁺ 291.66, found 291.80.

### Example 175

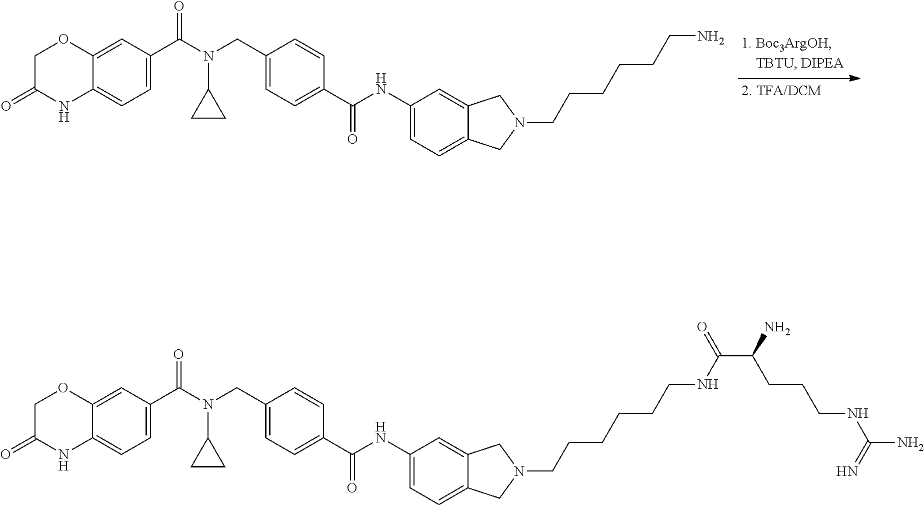 [SMILES: NCCCCCCN1Cc2ccc(NC(=O)c3ccc(CN(C(=O)c4ccc5c(c4)OCC(=O)N5)C4CC4)cc3)cc2C1 ; N=C(N)NCCC[C@H](N)C(=O)NCCCCCCN1Cc2ccc(NC(=O)c3ccc(CN(C(=O)c4ccc5c(c4)OCC(=O)N5)C4CC4)cc3)cc2C1]

To a 2-dram vial were added (Z)—N2,Nw,Nw′-tris(tert-butoxycarbonyl)-L-arginine (6.4 mg 1.1 Eq, 14 μmol), 2-(1H-benzo[d][1,2,3]triazol-1-yl)-1,1,3,3-tetramethylisouronium tetrafluoroborate (5.2 mg, 1.3 Eq, 16 μmol), N-ethyl-N-isopropylpropan-2-amine (8.0 mg, 11 μL, 5 Eq, 62 μmol), and DMF (0.2 mL). The reaction was left to stir at room temperature for 15 minutes. To the vial was then added N-(4-((2-(6-aminohexyl)isoindolin-5-yl)carbamoyl)benzyl)-N-cyclopropyl-3-oxo-3,4-dihydro-2H-benzo[b][1,4]oxazine-7-carboxamide bis(2,2,2-trifluoroacetate) (10 mg, 1 Eq, 12 μmol) dissolved in DMF (0.2 mL), and the reaction was left to stir overnight.

The reaction was quenched with distilled water and extracted 3 times ethyl acetate. The combined organic layers were washed twice with water, once with saturated sodium bicarbonate, and once with brine, then dried by passage through a phase separator and concentrated to a clear residue. To the residue was added 50% TFA in DCM, which was stirred until disappearance of starting material as monitored by LCMS. The reaction was concentrated, co-evaporated with methanol, and purified by reverse phase chromatography (10-100% methanol in water+0.1% TFA) to provide (S)—N-(4-((2-(6-(2-amino-5-guanidinopentanamido)hexyl)isoindolin-5-yl)carbamoyl) benzyl)-N-cyclopropyl-3-oxo-3,4-dihydro-2H-benzo[b][1,4]oxazine-7-carboxamide tris (2,2,2-trifluoroacetate) (8.83 mg, 8.18 μmol, 66%) as a white solid. ¹H NMR (400 MHz, Methanol-d₄) δ 7.98-7.91 (m, 3H), 7.63 (dd, J=8.3, 1.9 Hz, 1H), 7.50 (d, J=7.9 Hz, 2H), 7.40 (d, J=8.4 Hz, 1H), 7.20 (d, J=8.5 Hz, 1H), 7.17 (s, 1H), 6.96 (d, J=8.0 Hz, 1H), 4.83 (s, 2H), 4.62 (s, 2H), 3.86 (t, J=6.6 Hz, 1H), 3.49-3.40 (m, 2H), 3.29-3.21 (m, 4H), 2.88-2.79 (m, 1H), 1.95-1.81 (m, 4H), 1.72-1.64 (m, 2H), 1.64-1.55 (m, 2H), 1.53-1.41 (m, 4H), 0.70-0.61 (m, 2H), 0.61-0.51 (m, 2H). LCMS (ESI, +ve mode) expected m/z for [M+H]⁺ 738.41, found 738.30; expected m/z for [M+2H]²⁺ 369.71, found 369.80; expected m/z for [M+3H]³+246.81, found 246.95.

### Example 176

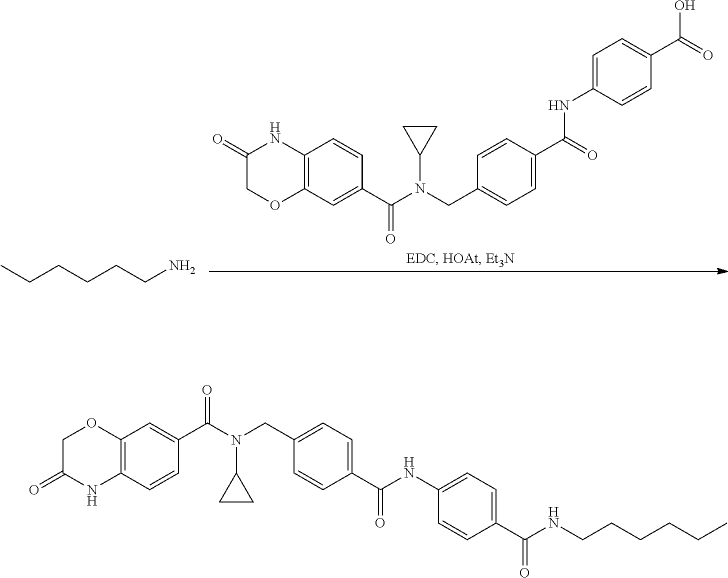

To a 2-dram vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), 3H-[1,2,3]triazolo[4,5-b]pyridin-3-ol (21 mg, 1.5 Eq, 0.15 mmol), 3-(((ethylimino)methylene)amino)-N,N-dimethylpropan-1-amine hydrochloride (30 mg, 1.5 Eq, 0.15 mmol), and dmf (0.5 mL). The reaction was left to stir at room temperature for 30 minutes. To the vial was then added hexan-1-amine (16 mg, 20 μL, 1.5 Eq, 0.15 mmol), followed by triethylamine (31 mg, 3 Eq, 0.31 mmol), and the reaction was left to stir overnight.

The next day, the reaction was diluted with water and the white precipitate formed was filtered off, then recrystallized from absolute ethanol to provide N-cyclopropyl-N-(4-((4-(hexylcarbamoyl)phenyl)carbamoyl)benzyl)-3-oxo-3,4-dihydro-2H-benzo[b][1,4]oxazine-7-carboxamide (16.83 mg, 29.60 μmol, 29%) as a white solid. ¹H NMR (400 MHz, DMSO-d₆) δ 10.87 (s, 1H), 10.41 (s, 1H), 8.34 (t, J=5.7 Hz, 1H), 7.96 (d, J=8.3 Hz, 2H), 7.90-7.80 (m, 4H), 7.46 (d, J=7.9 Hz, 2H), 7.19 (dd, J=8.1, 1.8 Hz, 1H), 7.15 (d, J=1.7 Hz, 1H), 6.93 (d, J=8.0 Hz, 1H), 4.72 (s, 2H), 4.62 (s, 2H), 3.24 (q, J=6.7 Hz, 2H), 2.80 (s, 1H), 1.56-1.46 (m, 2H), 1.36-1.22 (m, 6H), 0.87 (t, J=6.8 Hz, 3H), 0.59-0.51 (m, 2H), 0.51-0.44 (m, 2H). ¹³C NMR (101 MHz, DMSO-d₆) δ 165.57, 165.52, 164.85, 142.48, 142.33, 141.64, 133.44, 131.79, 129.57, 128.38, 128.05, 127.81, 127.21, 121.86, 119.33, 115.35, 115.15, 66.74, 31.04, 29.16, 26.19, 22.07, 13.93. LCMS (ESI, +ve mode) expected m/z for [M+H] 569.28, found 569.20.

### Example 177

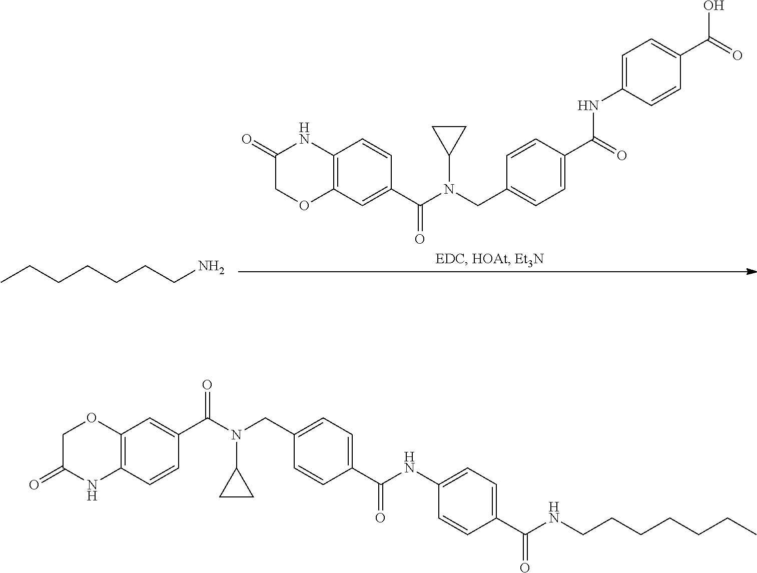

To a 2-dram vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), 3-(((ethylimino)methylene)amino)-N,N-dimethylpropan-1-amine hydrochloride (30 mg, 1.5 Eq, 0.15 mmol), 3H-[1,2,3]triazolo[4,5-b]pyridin-3-ol (21 mg, 1.5 Eq, 0.15 mmol), and dmf (0.5 mL). The reaction was left to stir at room temperature for 30 minutes. To the vial was then added heptan-1-amine (18 mg, 23 μL, 1.5 Eq, 0.15 mmol), followed by triethylamine (31 mg, 3 Eq, 0.31 mmol), and the reaction was left to stir overnight.

The next day, the reaction was diluted with water and the white precipitate formed was isolated by filtration, then recrystallized from absolute ethanol to provide N-cyclopropyl-N-(4-((4-(heptylcarbamoyl)phenyl)carbamoyl)benzyl)-3-oxo-3,4-dihydro-2H-benzo[b][1,4]oxazine-7-carboxamide (10.73 mg, 18.41 μmol, 18%) as a white solid. ¹H NMR (400 MHz, DMSO-d₆) δ 10.87 (s, 1H), 10.41 (s, 1H), 8.34 (t, J=5.6 Hz, 1H), 7.96 (d, J=8.5 Hz, 2H), 7.89-7.80 (m, 4H), 7.46 (d, J=7.9 Hz, 2H), 7.19 (dd, J=8.0, 1.7 Hz, 1H), 7.15 (d, J=1.7 Hz, 1H), 6.93 (d, J=8.1 Hz, 1H), 4.72 (s, 2H), 4.62 (s, 2H), 3.24 (q, J=6.6 Hz, 2H), 2.80 (s, 1H), 1.57-1.46 (m, 2H), 1.34-1.22 (m, 8H), 0.86 (t, J=7.0 Hz, 3H), 0.60-0.52 (m, 2H), 0.52-0.42 (m, 2H). ¹³C NMR (101 MHz, DMSO-d₆) δ 165.57, 165.52, 164.85, 142.48, 141.64, 133.44, 129.57, 128.38, 128.05, 127.80, 127.21, 121.86, 119.33, 66.73, 31.26, 29.19, 28.47, 26.49, 22.07, 13.96. LCMS (ESI, +ve mode) expected m/z for [M+H] 583.29, found 583.20.

### Example 178

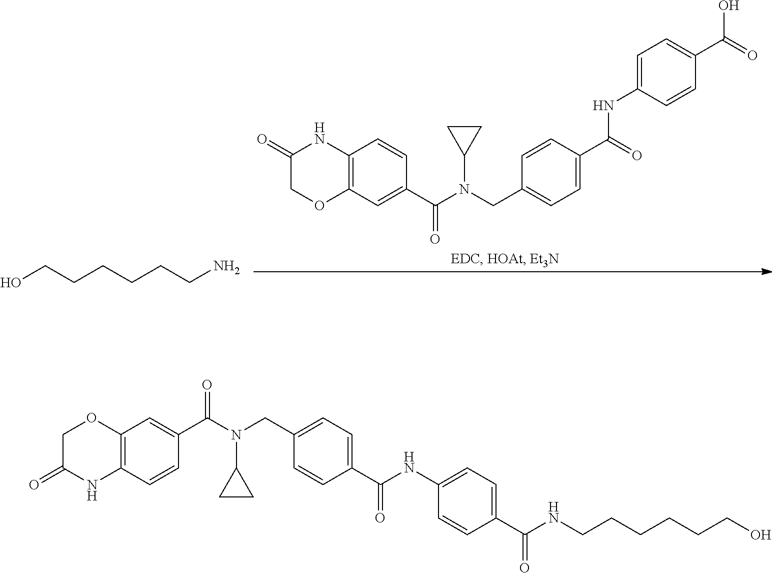 [SMILES: O=C1COc2cc(C(=O)N(Cc3ccc(C(=O)Nc4ccc(C(=O)O)cc4)cc3)C3CC3)ccc2N1 ; NCCCCCCO ; O=C1COc2cc(C(=O)N(Cc3ccc(C(=O)Nc4ccc(C(=O)NCCCCCCO)cc4)cc3)C3CC3)ccc2N1]

To a 2-dram vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), 3-(((ethylimino)methylene)amino)-N,N-dimethylpropan-1-amine hydrochloride (30 mg, 1.5 Eq, 0.15 mmol), 3H-[1,2,3]triazolo[4,5-b]pyridin-3-ol (21 mg, 1.5 Eq, 0.15 mmol), and dmf (0.5 mL). The reaction was left to stir at room temperature for 30 minutes. To the vial was then added 6-aminohexan-1-ol (18 mg, 1.5 Eq, 0.15 mmol) dissolved in dmf (0.5 mL), followed by triethylamine (31 mg, 43 μL, 3 Eq, 0.31 mmol), and the reaction was left to stir overnight.

The next day, the reaction was diluted with water and the white precipitate formed was isolated by centrifugation, washed 5 times with distilled water, and dried to provide N-cyclopropyl-N-(4-((4-((6-hydroxyhexyl)carbamoyl)phenyl)carbamoyl)benzyl)-3-oxo-3,4-dihydro-2H-benzo[b][1,4]oxazine-7-carboxamide (11.20 mg, 19.16 μmol, 19%) as a white solid. ¹H NMR (400 MHz, DMSO-d₆) δ 10.87 (s, 1H), 10.41 (s, 1H), 8.34 (t, J=5.6 Hz, 1H), 7.96 (d, J=8.3 Hz, 2H), 7.90-7.80 (m, 4H), 7.46 (d, J=7.9 Hz, 2H), 7.19 (dd, J=8.0, 1.8 Hz, 1H), 7.15 (d, J=1.7 Hz, 1H), 6.93 (d, J=8.1 Hz, 1H), 4.72 (s, 2H), 4.62 (s, 2H), 4.34 (t, J=5.2 Hz, 1H), 3.38 (q, J=6.4 Hz, 2H), 3.24 (q, J=6.6 Hz, 2H), 2.80 (s, 1H), 1.56-1.47 (m, 2H), 1.47-1.38 (m, 2H), 1.36-1.27 (m, 4H), 0.60-0.51 (m, 2H), 0.51-0.43 (m, 2H). ¹³C NMR (101 MHz, DMSO-d₆) δ 165.57, 165.53, 164.85, 142.48, 142.33, 141.64, 133.44, 129.57, 128.38, 128.06, 127.81, 127.21, 119.33, 115.36, 115.15, 66.74, 60.67, 32.51, 29.27, 26.45, 25.30. LCMS (ESI, +ve mode) expected m/z for [M+H] 585.27, found 585.20.

### Example 179

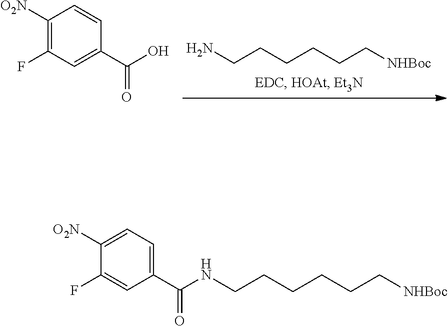 [SMILES: O=C(O)c1ccc([N+](=O)[O-])c(F)c1 ; CC(C)(C)OC(=O)NCCCCCCN ; CC(C)(C)OC(=O)NCCCCCCNC(=O)c1ccc([N+](=O)[O-])c(F)c1]

To a scintillation vial was added 3-fluoro-4-nitrobenzonic acid (0.20 g, 1.1 Eq, 1.1 mmol), EDC (0.29 g, 1.5 Eq, 1.5 mmol), HOAt (0.20 g, 1.5 Eq, 1.5 mmol), and DCM (3 mL). The reaction was left to stir at room temperature for 30 minutes. To the vial was then added 1-((tert-Butoxycarbonyl)amino)-6-aminohexane (0.22 g, 0.22 mL, 1 Eq, 1.0 mmol) dissolved in DCM (2 mL), followed by triethylamine (0.30 g, 0.42 mL, 3 Eq, 3.0 mmol), and the reaction was left to stir overnight.

The next day, the reaction was partitioned between water and DCM. The layers were separated, and the aqueous layer was extracted twice more with DCM. The combined organic layers were washed once with 0.5M citric acid, once with water, once with saturated sodium bicarbonate, and once with brine, then dried by passage through a phase separator and concentrated to an off-white residue. Normal phase chromatography over silica gel (0-50% ethyl acetate in hexanes) provided tert-butyl (6-(3-fluoro-4-nitrobenzamido)hexyl)carbamate (350 mg, 913 μmol, 91%) as a white solid. ¹H NMR (400 MHz, Chloroform-d) δ 8.06 (dd, J=8.5, 7.2 Hz, 1H), 7.89-7.72 (m, 2H), 7.46-7.32 (m, 1H), 4.70 (s, 1H), 3.41 (q, J=6.5 Hz, 2H), 3.10 (q, J=6.5 Hz, 2H), 1.60 (p, J=6.8 Hz, 2H), 1.45 (p, J=6.8 Hz, 2H), 1.39 (s, 9H), 1.37-1.25 (m, 4H). ¹³C NMR (101 MHz, Chloroform-d) δ 164.18, 156.68, 156.59, 154.04, 141.78, 141.71, 138.78, 138.70, 126.33, 126.31, 123.24, 123.19, 117.87, 117.64, 79.34, 39.58, 30.17, 28.99, 28.46, 25.48, 25.16, 14.25. ¹⁹F NMR (376 MHz, Chloroform-d) 6-116.41 (dd, J=11.4, 7.3 Hz).

### Example 180

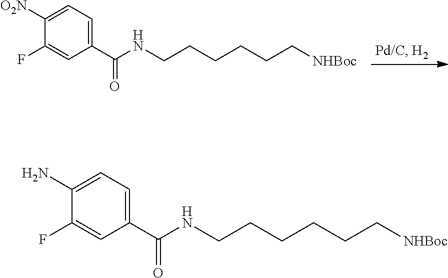 [SMILES: CC(C)(C)OC(=O)NCCCCCCNC(=O)c1ccc([N+](=O)[O-])c(F)c1 ; CC(C)(C)OC(=O)NCCCCCCNC(=O)c1ccc(N)c(F)c1]

To a round bottomed flask were added tert-butyl (6-(3-fluoro-4-nitrobenzamido)hexyl)carbamate (350 mg, 1 Eq, 913 μmol) and Methanol (10 mL). The flask was evacuated and backfilled with nitrogen three times, then Pd/C (35 mg, 0.36 Eq, 0.33 mmol) was added. The flask was again evacuated and backfilled with nitrogen three times, and the nitrogen inlet was replaced with a balloon of hydrogen gas. The reaction was stirred at room temperature under hydrogen atmosphere for 18 hours. The mixture was filtered through Celite and the filtrate concentrated to furnish tert-butyl (6-(4-amino-3-fluorobenzamido)hexyl)carbamate in essentially quantitative yield as a white solid that was used without further purification. ¹H NMR (400 MHz, Methanol-d₄) δ 7.44 (t, J=11.8 Hz, 2H), 6.79 (t, J=8.8 Hz, 1H), 3.29 (s, 2H), 3.00 (d, J=6.9 Hz, 2H), 1.66-1.44 (m, 4H), 1.40 (s, 9H), 1.33 (s, 4H). ¹³C NMR (101 MHz, cd₃od) δ 169.09, 158.51, 152.97, 150.60, 140.85, 140.72, 125.15, 125.12, 123.93, 123.87, 116.53, 116.49, 115.24, 115.04, 79.74, 41.19, 40.79, 30.86, 30.50, 28.78, 27.67, 27.47. ¹⁹F NMR (376 MHz, Methanol-d₄) δ −137.79 (t, J=10.4 Hz).

### Example 181

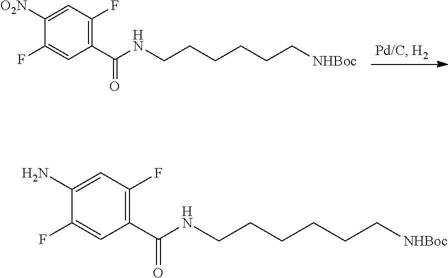 [SMILES: CC(C)(C)OC(=O)NCCCCCCNC(=O)c1cc(F)c([N+](=O)[O-])cc1F ; CC(C)(C)OC(=O)NCCCCCCNC(=O)c1cc(F)c(N)cc1F]

To a round bottomed flask were added tert-butyl (6-(2,5-difluoro-4-nitrobenzamido)hexyl)carbamate (373 mg, 1 Eq, 929 μmol) and Methanol (10 mL). The flask was evacuated and backfilled with nitrogen three times, then Pd/C (37 mg, 10% Wt, 0.037 Eq, 35 μmol) was added. The flask was again evacuated and backfilled with nitrogen three times, and the nitrogen inlet was replaced with a balloon of hydrogen gas. The reaction was stirred at room temperature under hydrogen atmosphere for 18 hours. The mixture was filtered through Celite and the filtrate concentrated to furnish tert-butyl (6-(4-amino-2,5-difluorobenzamido)hexyl)carbamate (341 mg, 918 μmol, 98.8%) as a white solid that was used without further purification. ¹H NMR (400 MHz, Methanol-d₄) δ 7.38 (dd, J=11.9, 6.8 Hz, 1H), 6.52 (dd, J=13.1, 7.1 Hz, 1H), 3.34 (t, J=7.1 Hz, 2H), 3.02 (t, J=7.0 Hz, 2H), 1.58 (p, J=7.1 Hz, 2H), 1.51-1.44 (m, 2H), 1.42 (s, 9H), 1.39-1.31 (m, 4H).

### Example 182

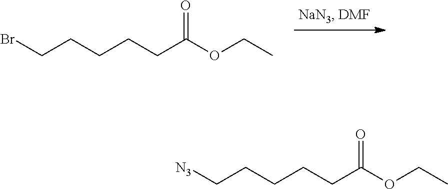

To a flask were added ethyl 6-bromohexanoate (1.1 g, 0.89 mL, 1 Eq, 5.0 mmol), dmf (10 mL), and sodium azide (0.42 g, 1.3 Eq, 6.5 mmol). The reaction vessel was capped and heated to 70° C. overnight. The next day, the reaction was quenched with water and extracted 3 times with ether. The combined organic layers were washed 3 times with water and once with brine, dried over MgSO4, and concentrated to provide ethyl 6-azidohexanoate (833.5 mg, 4.500 mmol, 90%) as a clear oil that was used without further purification. ¹H NMR (400 MHz, Chloroform-d) δ 4.13 (q, J=7.1 Hz, 2H), 3.27 (t, J=6.9 Hz, 2H), 2.31 (t, J=7.4 Hz, 2H), 1.70-1.57 (m, 4H), 1.46-1.36 (m, 2H), 1.26 (t, J=7.1 Hz, 3H). ¹³C NMR (101 MHz, Chloroform-d) δ 173.63, 60.45, 51.38, 34.26, 28.70, 26.38, 24.61, 14.38.

### Example 183

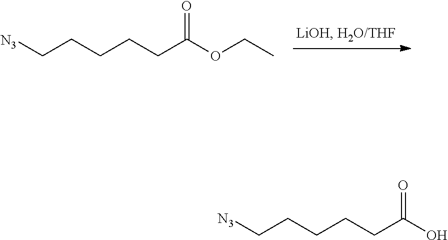

To a round-bottom flask were added ethyl 6-azidohexanoate (833 mg, 1 Eq, 4.50 mmol), THF (28 mL), and lithium hydroxide hydrate (566 mg, 3 Eq, 13.5 mmol) dissolved in water (7 mL). The reaction was stirred at room temperature for 24 hours, then extracted 5 times with 20 mL portions of diethyl ether. The aqueous layer was then acidified to pH 2 with 1M KHSO4 and extracted 3 times with ethyl acetate. The combined organic layers were washed once with brine, then dried over sodium sulfate and concentrated to provide 6-azidohexanoic acid (665 mg, 4.23 mmol, 94.1%) as a clear oil, which was used without further purification. ¹H NMR (400 MHz, Chloroform-d) δ 3.28 (t, J=6.8 Hz, 2H), 2.38 (t, J=7.4 Hz, 2H), 1.73-1.56 (m, 4H), 1.49-1.38 (m, 2H). ¹³C NMR (101 MHz, Chloroform-d) δ 179.70, 51.34, 33.91, 28.68, 26.30, 24.29.

### Example 184

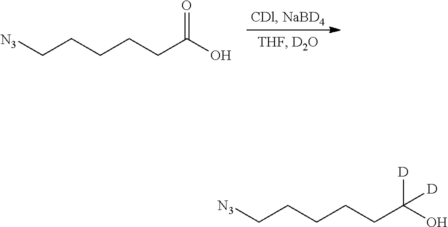 [SMILES: [N-]=[N+]=NCCCCCC(=O)O ; [2H]C([2H])(O)CCCCCN=[N+]=[N-]]

To a flask were added 6-azidohexanoic acid (0.16 g, 1 Eq, 1.0 mmol) and THF (4 mL). Next, CDI (0.24 g, 1.5 Eq, 1.5 mmol) was added cautiously. The reaction was stirred at room temperature for 10 minutes, then cooled in an ice bath. A solution of Sodium tetrahydroborate-d4 (84 mg, 78 μL, 2 Eq, 2.0 mmol) in D2O (2 mL) was added dropwise, and the reaction stirred for 2 hours at this temperature before being quenched by the addition of 2M HCl. The aqueous layer was extracted 3 times with ethyl acetate, and the combined organic layers were washed once with saturated sodium bicarbonate and once with brine, then dried over sodium sulfate and concentrated to provide 6-azidohexan-1,1-d2-1-ol (124.2 mg, 855.4 μmol, 86%) as a clear oil, which was used without further purification. ¹H NMR (400 MHz, Chloroform-d) δ 3.23 (t, J=6.9 Hz, 2H), 2.25 (s, 1H), 1.63-1.45 (m, 4H), 1.42-1.28 (m, 4H). ¹³C NMR (101 MHz, Chloroform-d) δ 77.48, 77.16, 76.84, 61.81 (p, J=21.5 Hz), 51.38, 32.28, 28.80, 26.53, 25.29.

### Example 185

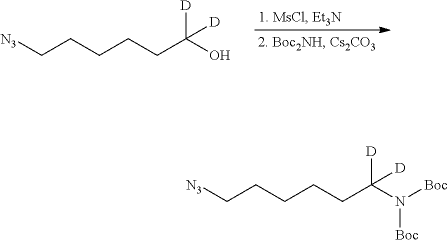 [SMILES: [2H]C([2H])(O)CCCCCN=[N+]=[N-] ; [2H]C([2H])(CCCCCN=[N+]=[N-])N(C(=O)OC(C)(C)C)C(=O)OC(C)(C)C]

To a flask were added 6-azidohexan-1,1-d2-1-ol (124 mg, 1 Eq, 854 μmol), DCM (5 mL), and triethylamine (156 mg, 214 μL, 1.8 Eq, 1.54 mmol). The flask was cooled in an ice bath, and mesyl-C1 (137 mg, 93.2 μL, 1.4 Eq, 1.20 mmol) was added dropwise. The reaction vessel was capped and left to stir overnight while coming to room temperature.

The next day, the reaction was quenched with 1M HCl and extracted 3 times with DCM. The combined organic extracts were washed once with water, once with saturated sodium bicarbonate, and once with brine, then dried over sodium sulfate and concentrated to a residue. NMR confirmed the formation of the mesylate.

To the crude mesylate in a flask were added Boc2NH (223 mg, 1.2 Eq, 1.02 mmol), Cs2CO3 (417 mg, 1.5 Eq, 1.28 mmol), and DMF (2 mL). The reaction was sealed and heated to 70° C. overnight. The next day, the reaction was quenched with water and extracted 3 times with ethyl acetate. The combined organic extracts were washed 3 times with water and once with brine, then dried over sodium sulfate and concentrated to a residue. Normal phase chromatography over silica gel (0-20% ethyl acetate in hexanes) provided tert-butyl (6-azidohexyl-1,1-d2)(tert-butoxycarbonyl)carbamate (249.5 mg, 724.3 μmol, 84.8% over 2 steps) as a clear oil. ¹H NMR (400 MHz, Chloroform-d) δ 3.25 (t, J=6.9 Hz, 2H), 1.67-1.54 (m, 4H), 1.50 (s, 18H), 1.43-1.24 (m, 4H). ¹³C NMR (101 MHz, Chloroform-d) δ 152.87, 82.23, 51.53, 28.95, 28.80, 28.24, 26.59, 26.47.

### Example 186

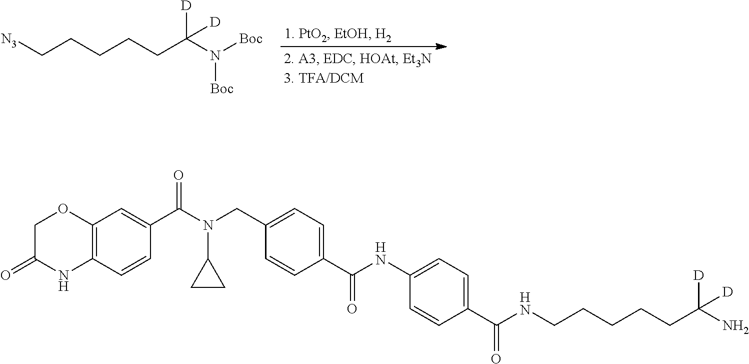 [SMILES: [2H]C([2H])(CCCCCN=[N+]=[N-])N(C(=O)OC(C)(C)C)C(=O)OC(C)(C)C ; [2H]C([2H])(N)CCCCCNC(=O)c1ccc(NC(=O)c2ccc(CN(C(=O)c3ccc4c(c3)OCC(=O)N4)C3CC3)cc2)cc1]

To a flask were added tert-butyl (6-azidohexyl-1,1-d2)(tert-butoxycarbonyl)carbamate (100 mg, 1 Eq, 290 μmol) and EtOH (5 mL). A nitrogen line was attached, and the flask evacuated and backfilled with nitrogen 3 times before platinum(IV) oxide (10 mg, 0.15 Eq, 44 μmol) was added. The flask was then evacuated and backfilled another 3 times before the nitrogen line was replaced with a balloon of hydrogen and the reaction left to stir overnight.

The next day, TLC analysis indicated complete consumption of starting material. The reaction was filtered through a pad of Celite, rinsing with methanol. The solvents were removed on the rotovap and the residue used without further purification.

To a vial were added 4-(4-((N-cyclopropyl-3-oxo-3,4-dihydro-2H-benzo[b][1,4]oxazine-7-carboxamido)methyl)benzamido)benzoic acid (50 mg, 1 Eq, 0.10 mmol), 3-(((ethylimino)methylene)amino)-N,N-dimethylpropan-1-amine hydrochloride (30 mg, 1.5 Eq, 0.15 mmol), HOAt(21 mg, 1.5 Eq, 0.15 mmol), and DMF (0.5 mL). The reaction was stirred for 20 minutes to activate the acid, then tert-butyl (6-aminohexyl-1,1-d2)(tert-butoxycarbonyl)carbamate (92.4 mg 2.8 Eq, 290 μmol) in DMF was added, followed by triethylamine (31 mg, 43 μL, 3 Eq, 0.31 mmol). The reaction was left to stir overnight.

The next day, the reaction was quenched with water and extracted 3 times with ethyl acetate. The combined organic fractions were washed once with 0.5 M citric acid, once with water, once with saturated sodium bicarbonate, and once with brine, then dried over sodium sulfate, filtered, and concentrated to a residue. To the residue was added 20% TFA in DCM, which was stirred at room temperature overnight. The next day, the volatiles were removed by rotovap and the residue co-evaporated with methanol. Purification by reverse phase chromatography (10-100% methanol in water+0.1% TFA) provided N-(4-((4-((6-aminohexyl-6,6-d2)carbamoyl)phenyl)carbamoyl)benzyl)-N-cyclopropyl-3-oxo-3,4-dihydro-2H-benzo[b][1,4]oxazine-7-carboxamide 2,2,2-trifluoroacetate (50.84 mg, 72.66 μmol, 71% over 3 steps) as a white solid following lyophilization. ¹H NMR (400 MHz, Methanol-d₄) δ 7.95 (d, J=8.3 Hz, 2H), 7.87-7.80 (m, 4H), 7.48 (d, J=7.9 Hz, 2H), 7.19 (dd, J=8.1, 1.7 Hz, 1H), 7.16 (s, 1H), 6.95 (d, J=8.1 Hz, 1H), 4.81 (s, 2H), 4.61 (s, 2H), 3.39 (t, J=7.1 Hz, 2H), 2.85-2.76 (m, 1H), 1.72-1.59 (m, 4H), 1.51-1.37 (m, 4H), 0.71-0.59 (m, 2H), 0.59-0.46 (m, 2H). ¹³C NMR (101 MHz, Methanol-d₄) δ 169.56, 168.58, 167.33, 144.62, 143.61, 143.14, 135.08, 131.12, 129.92, 129.16, 129.06, 128.88, 123.22, 121.30, 116.90, 116.72, 68.13, 49.85, 40.66, 30.31, 28.25, 27.40, 26.95, 10.50. LCMS (ESI, +ve mode)expected m/z for C33H36D2N5O5+[M+H] 586.30, found 586.20.

That which is claimed is:

1. A compound of Formula (I): 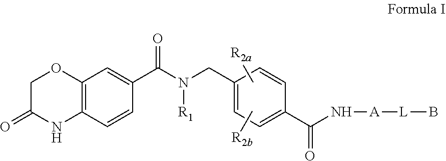
or an enantiomer, an enantiomeric mixture, or a pharmaceutically acceptable salt thereof; wherein:
R₁ is -cyclopropyl or -isopropyl;
R₂ₐ and R_{2b} are independently selected from hydrogen, C1-C6 alkyl, C1-C6 haloalkyl, C1-C6 alkoxy, halogen, —CN, —NH₂, and —OH;
A is absent or present, and when present is selected from 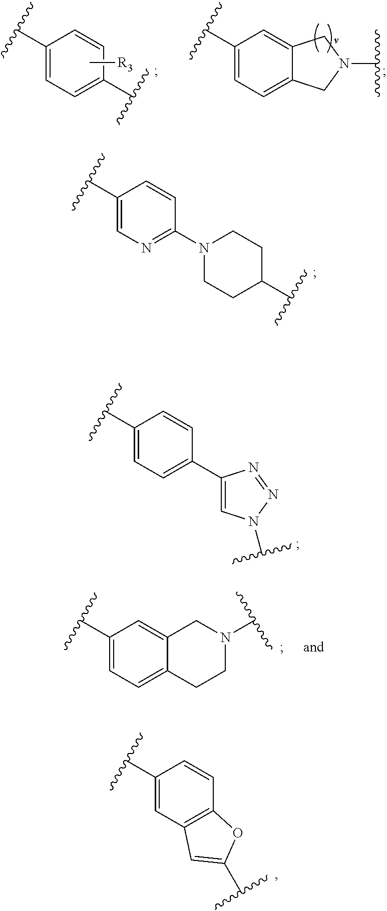
wherein R₃ is selected from hydrogen, C1-C6 alkyl, C1-C6 haloalkyl, C1-C6 alkoxy, halogen, —CN, —NH₂, and —OH, and v is 1 or 2;
L is 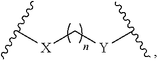
wherein X is selected from —C(═O)—, —CH₂—, C(═O)O—, —C≡C—, —O—, —S—, —NH—, —S(═O)—, —C(═O)NH—, —C(═O)NCH₃—, and 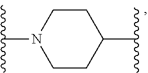
wherein Y is absent or is selected from —O—, —CH₂—, —NH—, —NCH₃—, —[OCH₂CH₂]ₘ—, and 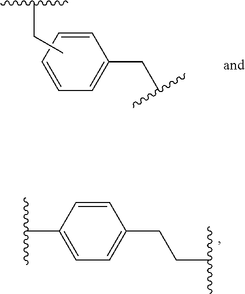
wherein m is an integer selected from 1, 2, 3, 4, and 5, and wherein n is an integer selected from 0, 1, 2, 3, 4, 5, 6, 7, 8, 9, and 10; and
B is selected from hydrogen, —NH₂, —NH(CH₃)—C(═O)(C1-C6 alkyl), —C(═O)(C1-C6 haloalkyl), C1-C6 alkyl, —O(C1-C6 alkyl), —COOH, —OH, —NH(C1-C6 alkyl), —N(C1-C6 alkyl)₂, 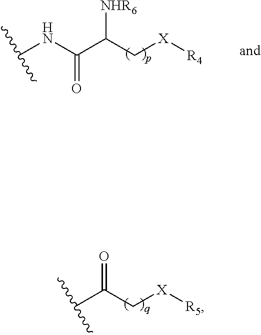
wherein p and q are an integer independently selected from 0, 1, 2, 3, 4, 5, 6, 7, 8, 9, and 10, X is selected from —CH₂—, —NH—, and —O—, R₆ is hydrogen or —C(═O)CH₃—, and R₄ and R₅ are independently selected from hydrogen, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃,
with the proviso that when A is 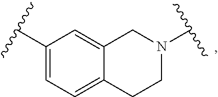 [SMILES: *c1ccc2c(c1)CN(*)CC2]
L is not 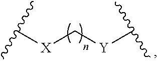
wherein X is —C(═O)NH—, n is 4, Y is —CH₂, and B is —NH₂.

2. The compound of claim 1, wherein R₁ is -cyclopropyl.

3. The compound of claim 1 or 2, wherein A is selected from

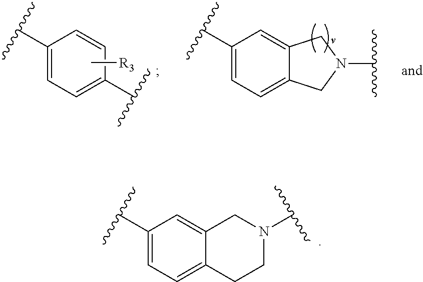

4. The compound of claim 3, wherein X is selected from —O—, —CH₂—, —C(═O)NH—, —C(═O)N(CH₃)—, —C(═O), and

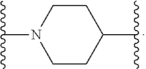 [SMILES: *C1CCN(*)CC1]

5. The compound of claim 4, wherein X is selected from —C(═O)NH and —C(═O).

6. The compound of claim 4, wherein R₂ₐ and R_{2b} are independently selected from hydrogen, halogen, and C1-C6 alkoxy.

7. The compound of claim 6, wherein the compound is a compound of Formula (II): 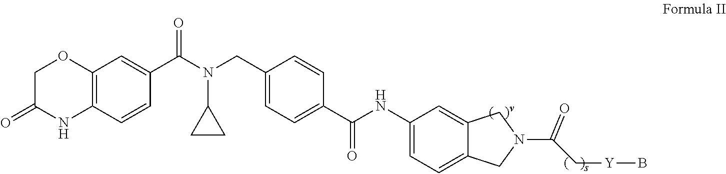
or an enantiomer, an enantiomeric mixture, or a pharmaceutically acceptable salt thereof; wherein:
v is 1 or 2;
s is an integer selected from 1, 2, 3, 4, 5, 6, 7, 8, 9, and 10;
Y is absent or is selected from —O—, —CH₂—, —NH—, —NCH₃—, —[OCH₂CH₂]ₘ—, 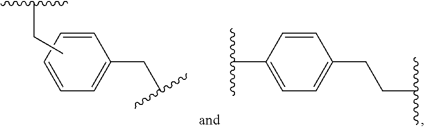
wherein m is an integer selected from 1, 2, 3, 4, and 5; and
B is selected from —H, —NH₂, —NH(CH₃), —C(═O)(C1-C6 alkyl), —C(═O)(C1-C6 haloalkyl), C1-C6 alkyl, —O(C1-C6 alkyl), —COOH, —OH, —N(C1-C6 alkyl), —N(C1-C6 alkyl)₂, and 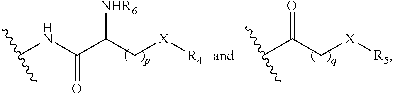
wherein p and q are independently an integer selected from 0, 1, 2, 3, 4, 5, 6, 7, 8, 9, and 10, X is selected from —CH₂—, —NH—, and —O—, R₆ is H or —C(═O)CH₃—, and R₄ and R₅ are independently selected from —H, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

8. The compound of claim 7, wherein Y is —CH₂— and s is selected from 3, 4 and 5.

9. The compound of claim 8, wherein B is selected from —NH₂ and 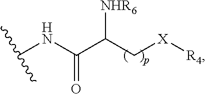
wherein p is an integer selected from 0, 1 and 2, X is —CH₂—, R₆ is H, and R₄ is selected from C5-C10 heteroraryl, and —NHC(═NH₂)NH₂.

10. The compound of claim 8, wherein s is 4 and B is —NH₂.

11. The compound of claim 7, wherein the compound is selected from

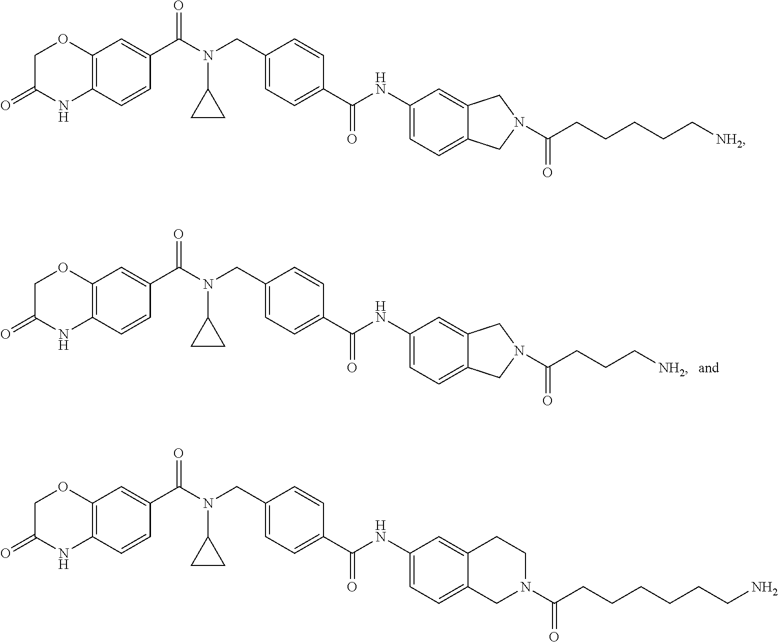 [SMILES: NCCCCCC(=O)N1Cc2ccc(NC(=O)c3ccc(CN(C(=O)c4ccc5c(c4)OCC(=O)N5)C4CC4)cc3)cc2C1 ; NCCCC(=O)N1Cc2ccc(NC(=O)c3ccc(CN(C(=O)c4ccc5c(c4)OCC(=O)N5)C4CC4)cc3)cc2C1 ; "and" ; NCCCCCCC(=O)N1CCc2cc(NC(=O)c3ccc(CN(C(=O)c4ccc5c(c4)OCC(=O)N5)C4CC4)cc3)ccc2C1]

12. The compound of claim 6, wherein the compound is a compound of Formula (III): 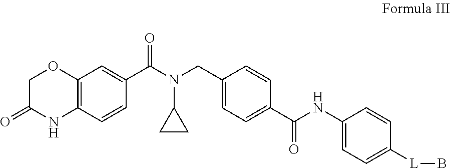
or an enantiomer, an enantiomeric mixture, or a pharmaceutically acceptable salt thereof; wherein:
L is 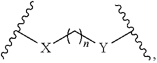
wherein X is selected from —C(═O)—, —CH₂—, C(═O)O—, —C≡C—, —O—, —S—, —NH—, —S(═O)—, —C(═O)NH—, —C(═O)NCH₃—, and 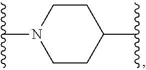
wherein Y is present or absent, and when present is selected from —O—, —CH₂—, —NH—, —NCH₃—, [OCH₂CH₂]ₘ—, 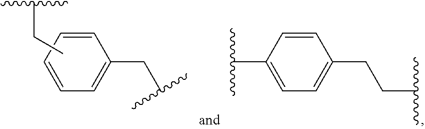
wherein m is an integer selected from 1, 2, 3, 4, and 5, and wherein n is selected from integer 0, 1, 2, 3, 4, 5, 6, 7, 8, 9, and 10; and
B is selected from hydrogen, —NH₂, —NH(CH₃)—C(═O)(C1-C6 alkyl), —C(═O)(C1-C6 haloalkyl), C1-C6 alkyl, —O(C1-C6 alkyl), —COOH, —OH, —NH(C1-C6 alkyl), —N(C1-C6 alkyl)₂, 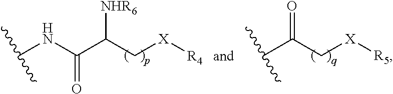
wherein p and q are independently an integer selected from 0, 1, 2, 3, 4, 5, 6, 7, 8, 9, and 10, X is selected from —CH₂—, —NH—, and —O—, R₆ is hydrogen or —C(═O)CH₃—, and R₄ and R₅ is independently selected from hydrogen, —C1-C6 alkyl, —C1-C6 haloalkyl, C5-C10 heteroraryl, C5-C10 aryl, C5-C10 heterocycloalkyl, C5-C10 cycloalkyl, —N(CH₃)C(═NH)NH₂, —N═C(NH₂)NH₂, and —N—C(═NH)NHCH₃.

13. The compound of claim 12, wherein X is selected from —O— and —C(═O)NH—.

14. The compound of claim 13, wherein n is an integer selected from 5, 6 and 7 and Y is —CH₂—.

15. The compound of claim 14, wherein B is selected from hydrogen, —OH, —NH₂, —NH(CH₃), and

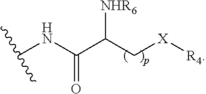

17. The compound of claim 15, wherein B is —NH₂.

18. The compound of claim 12, wherein the compound is selected from:

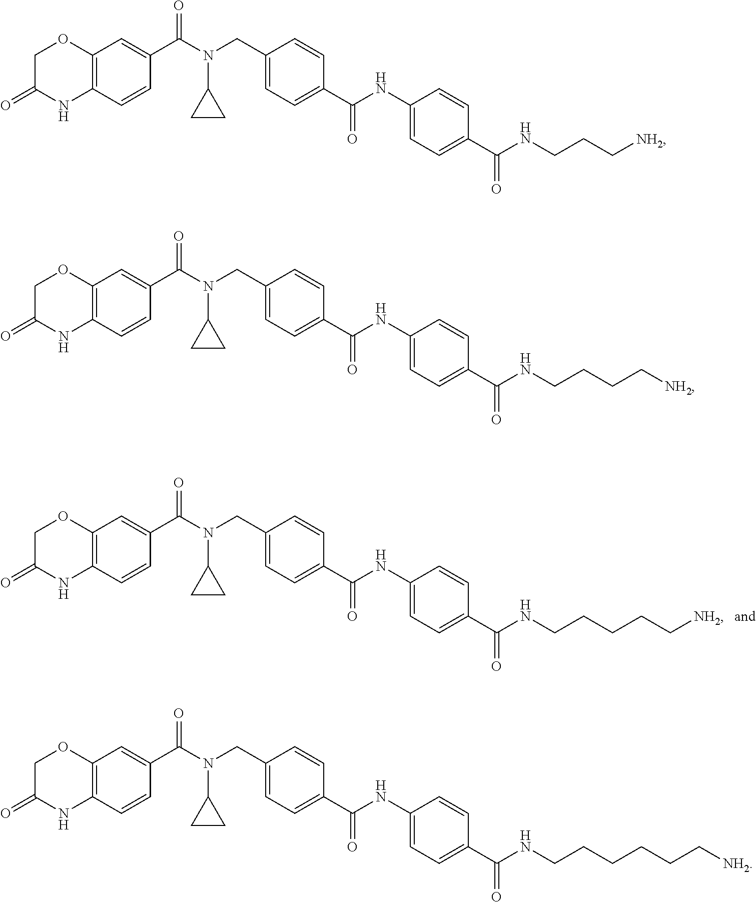 [SMILES: NCCCNC(=O)c1ccc(NC(=O)c2ccc(CN(C(=O)c3ccc4c(c3)OCC(=O)N4)C3CC3)cc2)cc1 ; NCCCCNC(=O)c1ccc(NC(=O)c2ccc(CN(C(=O)c3ccc4c(c3)OCC(=O)N4)C3CC3)cc2)cc1 ; NCCCCCNC(=O)c1ccc(NC(=O)c2ccc(CN(C(=O)c3ccc4c(c3)OCC(=O)N4)C3CC3)cc2)cc1 ; "and" ; NCCCCCCNC(=O)c1ccc(NC(=O)c2ccc(CN(C(=O)c3ccc4c(c3)OCC(=O)N4)C3CC3)cc2)cc1]

19. The compound of claim 15, wherein B is 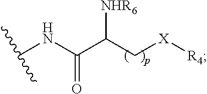
R₆ is H or C(═O)CH₃; p is an integer selected from 0, 1, 2, and 3; and R₄ is selected from —C1-C6 alkyl, —C1-C6 haloalkyl, C5-C10 aryl, C5-C10 heteroraryl, N(CH₃)C(═NH)NH₂, —N═C(NH₂)NH₂, and —N—C(═NH)NHCH₃.

20. The compound of claim 19, wherein R₆ is H, p is 3, and R₄ is selected from C5-C10 aryl, C5-C10 heteroraryl, N(CH₃)C(═NH)NH₂, —N═C(NH₂)NH₂, and —N—C(═NH)NHCH₃.

21. The compound of claim 20, wherein R₄—N═C(NH₂)NH₂.

22. The compound of claim 15, wherein the compound is selected from:

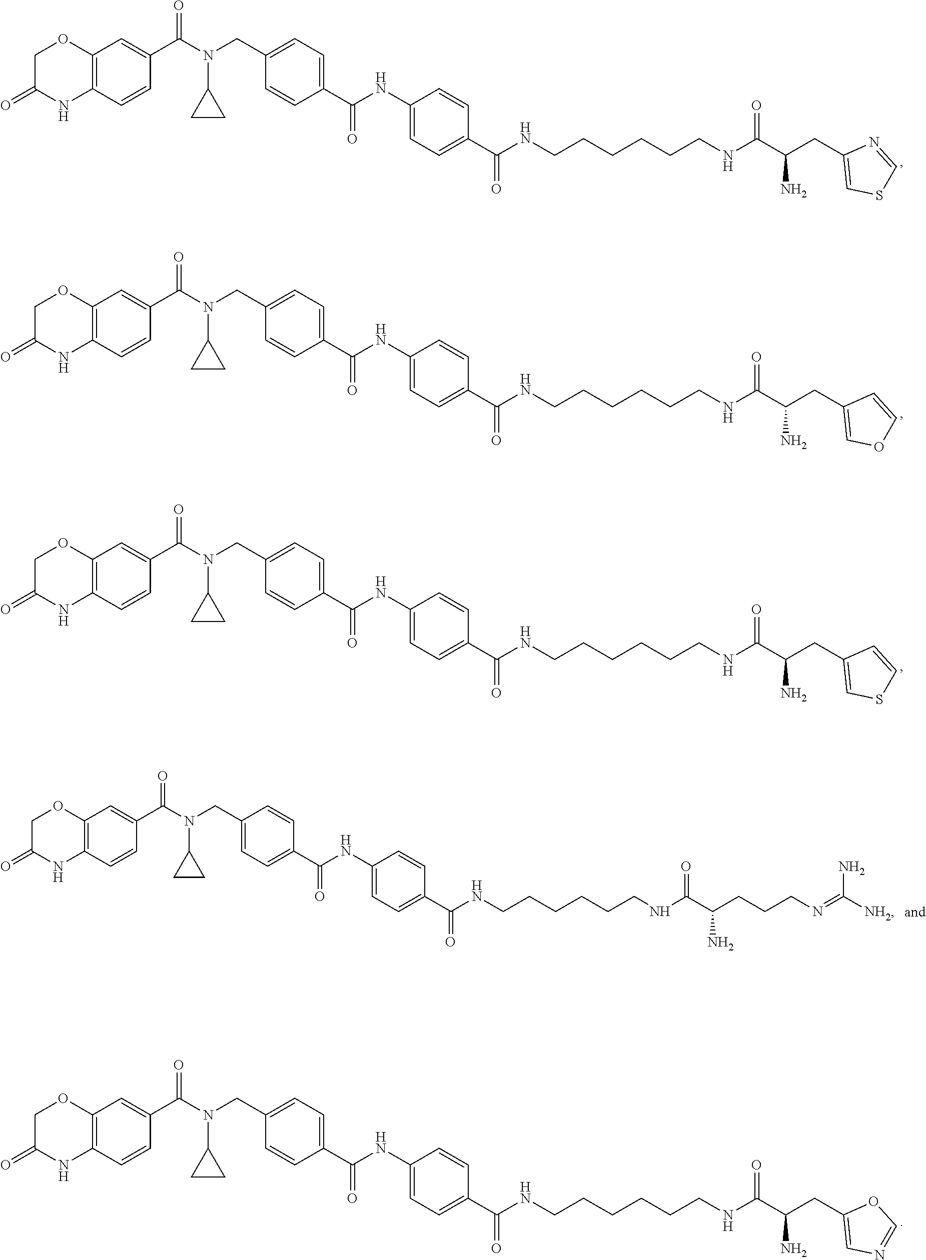 [SMILES: N[C@H](Cc1cscn1)C(=O)NCCCCCCNC(=O)c1ccc(NC(=O)c2ccc(CN(C(=O)c3ccc4c(c3)OCC(=O)N4)C3CC3)cc2)cc1 ; N[C@@H](Cc1ccoc1)C(=O)NCCCCCCNC(=O)c1ccc(NC(=O)c2ccc(CN(C(=O)c3ccc4c(c3)OCC(=O)N4)C3CC3)cc2)cc1 ; N[C@H](Cc1ccsc1)C(=O)NCCCCCCNC(=O)c1ccc(NC(=O)c2ccc(CN(C(=O)c3ccc4c(c3)OCC(=O)N4)C3CC3)cc2)cc1 ; NC(N)=NCCC[C@H](N)C(=O)NCCCCCCNC(=O)c1ccc(NC(=O)c2ccc(CN(C(=O)c3ccc4c(c3)OCC(=O)N4)C3CC3)cc2)cc1 ; "and" ; N[C@H](Cc1cnco1)C(=O)NCCCCCCNC(=O)c1ccc(NC(=O)c2ccc(CN(C(=O)c3ccc4c(c3)OCC(=O)N4)C3CC3)cc2)cc1]

23. A pharmaceutical composition comprising a compound according to any one of the preceding claims or a pharmaceutically acceptable salt thereof, and one or more pharmaceutically acceptable carrier(s).

24. The pharmaceutical composition of claim 23, wherein the compound is a prodrug.

25. A method for treating a disease or condition that is treatable by inhibition of nuclear SET-domain-containing protein (NDS2), the method comprising administering to a subject in need thereof a therapeutically effective amount of a compound of any one of the preceding claims or a pharmaceutical composition of claim 23.

26. The method of claim 25, wherein the disease is cancer.

27. The method if claim 26, wherein the cancer is selected from breast cancer, cervical cancer, skin cancer, ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia, non-Hodgkin's lymphoma, and pulmonary arterial hypertension.
